(19) United States
(12) Patent Application Publication
(10) Pub. No.: US 2024/0287070 A1
(43) Pub. Date: Aug. 29, 2024

(54) FUSED HETEROCYCLIC DERIVATIVES

(71) Applicant: Janssen Sciences Ireland Unlimited Company, Co Cork (IE)

(72) Inventors: Chunliang LU, Shanghai (CN); Xiaoyu LI, Shanghai (CN); Lianzhu LIU, Shanghai (CN); Gang DENG, Shanghai (CN); Zhiguo LIU, Shanghai (CN); Bingqing TANG, Anhui Province (CN); Wei CAI, Shanghai (CN); Zhanling CHENG, Shanghai (CN); Sandrine Céline GROSSE, Turnhout (BE); Koen VANDYCK, Paal-Beringen (BE); Edgar JACOBY, Vosselaar (BE); Tim Hugo Maria JONCKERS, Heist-op-den-Berg (BE); Pierre Jean-Marie Bernard RABOISSON, Wavre (BE); Scott D. KUDUK, Harleysville, PA (US); Lindsey Graham DERATT, North Wales, PA (US)

(21) Appl. No.: 18/565,725

(22) PCT Filed: Jun. 1, 2022
(86) PCT No.: PCT/CN2022/096535

§ 371 Date: Nov. 30, 2023

(30) Foreign Application Priority Data
Jun. 2, 2021 (WO) PCT/CN2021/097847
Apr. 29, 2022 (WO) PCT/CN2022/090238

(51) Int. Cl. C07D 471/14; A61K 31/4985; A61K 31/501; A61K 31/506; A61K 31/5377; A61K 45/06; A61P 31/20

(52) U.S. Cl. CPC C07D 471/14; A61K 31/4985; A61K 31/501; A61K 31/506; A61K 31/5377; A61K 45/06; A61P 31/20

(57) ABSTRACT

The application describes fused heterocycle derivative compounds, pharmaceutical compositions comprising these compounds, chemical processes for preparing these compounds and their use in the treatment of diseases associated with HBV infection.

This application claims priority to International Application PCT/CN2021/097847, filed on Jun. 2, 2021; and International Application PCT/CN2022/090238, filed on Apr. 29, 2022. Each disclosure is incorporated herein by reference in its entirety.

### FIELD

The application relates to fused heterocyclic derivative compounds, pharmaceutical compositions comprising these compounds, chemical processes for preparing these compounds and their use in the treatment of diseases associated with HBV infection.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant unmet worldwide medical problem, due to suboptimal treatment options and sustained rates of new infections in most parts of the developing world. Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress. Capsid structures also respond to environmental cues to allow un-coating after viral entry. Consistently, the appropriate timing of capsid assembly and dis-assembly, the appropriate capsid stability and the function of core protein have been found to be critical for viral infectivity.

The crucial function of HBV capsid proteins imposes stringent evolutionary constraints on the viral capsid protein sequence, leading to the observed low sequence variability and high conservation. Consistently, mutations in HBV capsid that disrupt its assembly are lethal, and mutations that perturb capsid stability severely attenuate viral replication. The high functional constraints on the multi-functional HBV core/capsid protein is consistent with a high sequence conservation, as many mutations are deleterious to function. Indeed, the core/capsid protein sequences are >90% identical across HBV genotypes and show only a small number of polymorphic residues. Resistance selection to HBV core/capsid protein binding compounds may therefore be difficult to select without large impacts on virus replication fitness.

Reports describing compounds that bind viral capsids and inhibit replication of HIV, rhinovirus and HBV provide strong pharmacological proof of concept for viral capsid proteins as antiviral drug targets.

There is a need in the art for therapeutic agents that can increase the suppression of virus production and that can treat, ameliorate, and/or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

In view of the clinical importance of HBV, the identification of compounds that can increase the suppression of virus production and that can treat, ameliorate, and/or prevent HBV infection represents an attractive avenue into the development of new therapeutic agents. Such compounds are provided herein.

### SUMMARY

The present disclosure is directed to the general and preferred embodiments defined, respectively, by the independent and dependent claims appended hereto, which are incorporated by reference herein. The present disclosure is directed to compounds capable of capsid assembly modulation. The compounds of the present disclosure may provide a beneficial balance of properties with respect to prior art compounds, e.g. they may display a different profile, display improved solubility, etc.

Thus, in particular, the present disclosure is directed to a compound of Formula (I):

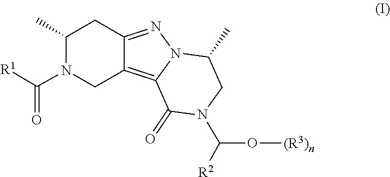

or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN; R² is selected from the group consisting of H, CHF₂, CF₃, C₁₋₆alkyl, C₁₋₆alkylOC₁₋₆alkyl, C₃₋₆cycloalkyl and CON(R_{S})₂; Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, oxo, and N(R_{S})₂, each of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂ is optionally substituted with 1, 2, 3, 4 or 5 substituents, each of said substituents independently selected from the group consisting of halo, hydroxyl, C₁₋₆alkyl and oxo;
R_{S} is each independently selected from the group consisting of H, C₁₋₆alkyl, CN, SOC₁₋₆alkyl, SO₂C₁₋₆alkyl, SO₂OH, C₃₋₆cycloalkyl;
or a pharmaceutically acceptable salt or a solvate thereof.

Further embodiments include pharmaceutically acceptable salts and solvates of compounds of Formula (I), and stereoisomeric and tautomeric forms of the compounds of Formula (I), as well as pharmaceutically acceptable salts thereof.

In embodiments, the compounds of Formula (I) are compounds selected from those species described or exemplified in the detailed description below.

The present disclosure is also directed to pharmaceutical compositions comprising one or more compounds of Formula (I), and pharmaceutically acceptable salts and solvates of compounds of Formula (I). Pharmaceutical compositions may further comprise one or more pharmaceutically acceptable excipients or one or more other agents or therapeutics.

The present disclosure is also directed to methods of using or uses of compounds of Formula (I). In embodiments, compounds of Formula (I) are used to treat or ameliorate hepatitis B viral (HBV) infection, increase the suppression of HBV production, interfere with HBV capsid assembly or other HBV viral replication steps or products thereof. The methods comprise administering to a subject in need of such method an effective amount of at least one compound of Formula (I), and pharmaceutically acceptable salts and solvates of compounds of Formula (I). Additional embodiments of methods of treatment are set forth in the detailed description.

### DETAILED DESCRIPTION

Additional embodiments, features, and advantages of the subject matter of the present disclosure will be apparent from the following detailed description of such disclosure and through its practice. For the sake of brevity, the publications, including patents, cited in this specification are herein incorporated by reference.

In one embodiment, provided herein are compounds of Formula (I),

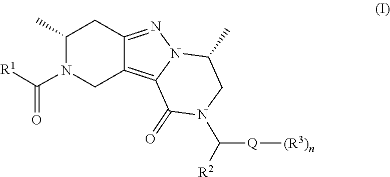

or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl, C₃₋₆cycloalkyl and CON(R_{S})₂;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, oxo, and N(R_{S})₂, each of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂ is optionally substituted with 1, 2, 3, 4 or 5 substituents, each of said substituents independently selected from the group consisting of halo, hydroxyl, C₁₋₆alkyl and oxo;
R_{S} is each independently selected from the group consisting of H, C₁₋₆alkyl, CN, SOC₁₋₆alkyl, SO₂C₁₋₆alkyl, SO₂OH, C₃₋₆cycloalkyl;

or a pharmaceutically acceptable salt or a solvate thereof.

In an embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN. Each of C₁₋₆alkyl, C₁₋₆alkoxyl and C₃₋₆cycloalkyl is optionally substituted with 1, 2, 3, 4 or 5 (e.g., 1, 2 or 3) substituents independently selected from the group consisting of halo (e.g., F).

In an embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃.

In another embodiment, R¹ is selected from the group consisting of phenyl, 5-membered heteroaryl and 6-membered heteroaryl, each of which is substituted with 1, 2 or 3 substituents, each of said substituents independently selected from the group consisting of halo, CN and CF₃.

In a further embodiment, R¹ is a ring selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃; preferably, each of said substituents independently selected from the group consisting of halo, CN and CF₃.

In a preferable embodiment, R¹ is a ring of phenyl. In a further embodiment, R¹ is a ring of phenyl, which is substituted with 1, 2 or 3 substituents, each of said substituents independently selected from the group consisting of halo, CN and CF₃.

In another preferable embodiment, R¹ is a ring of pyridyl. In a further embodiment, R¹ is a ring of pyridyl, which is substituted with 1, 2 or 3 substituents, each of said substituents independently selected from the group consisting of halo, CN and CF₃.

In an embodiment, the number of substituents on R¹ is 1 or 2, preferably 2.

In an embodiment, the structural unit

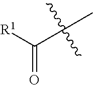

in Formula (I) of the present disclosure satisfies Formula (Ia)

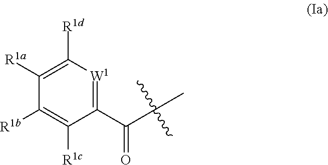

wherein R^{1a}, R^{1b}, R^{1c} and R^{1d} are each independently selected from the group consisting of hydrogen, halo, C₁₋₆alkyl, C₃₋₆cycloalkyl, CN, CF₃, CHF₂, OCHF₂ and OCF₃; W¹ is N or CH; with at least one of R^{1a}, R^{1b}, R^{1c} and R^{1d} not being hydrogen.

In a preferable embodiment, R^{1a} is halo; R^{1b} and R^{1d} are each independently selected from the group consisting of hydrogen, halo, cyano, CF₃, OCHF₂ and OCF₃; and R^{1c} is selected from the group consisting of hydrogen and halo. In another preferable embodiment, R^{1a} is halo; R^{1b} and R^{1d} are each independently selected from the group consisting of hydrogen, halo, cyano, and CF₃; and R^{1c} is selected from the group consisting of hydrogen and halo.

In an embodiment, W¹ is CH. In another embodiment, W¹ is N.

Preferably, the halo is Cl or F.

In another embodiment, Formula (Ia) satisfies Formula (Ia-1)

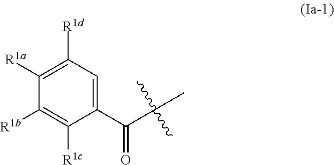

wherein R^{1a}, R^{1b}, R^{1c}, and R^{1d} are each independently selected from the group consisting of hydrogen, halo, C₁₋₆alkyl, C₃₋₆cycloalkyl, CN, CF₃, CHF₂, OCHF₂ and OCF₃, with at least one of R^{1a}, R^{1b}, R^{1c} and R^{1d} not being hydrogen.

In a preferable embodiment, R^{1a} is halo, R^{1b} and R^{1d} are each independently selected from the group consisting of H, halo, CF₃, cyano, OCHF₂ and OCF₃, R^{1c} is selected from the group consisting of hydrogen and halo. In another preferable embodiment, R^{1a} is halo, R^{1b} and R^{1d} are each independently selected from the group consisting of H, halo, CF₃ and cyano, R^{1c} is selected from the group consisting of hydrogen and halo.

Preferably, the halo is Cl or F.

In yet another embodiment, Formula (Ia) satisfies Formula (Ia-2)

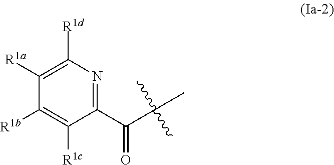

wherein R^{1a}, R^{1b}, R^{1c} and R^{1d} are each independently selected from the group consisting of hydrogen, halo, C₁₋₆alkyl, C₃₋₆cycloalkyl, CN, CF₃, CHF₂, OCHF₂ and OCF₃; with at least one of R^{1a}, R^{1b}, R^{1c} and R^{1d} not being hydrogen.

In a preferable embodiment, R^{1a} is halo, R^{1b} is selected from the group consisting of H and CF₃, R^{1c} is hydrogen and R^{1d} is selected from the group consisting of H and CF₃.

Preferably, the halo is Cl or F.

In yet another embodiment, Formula (Ia) satisfies Formula (Ia-3)

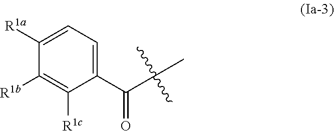

wherein R^{1a}, R^{1b}, and R^{1c}, each independently are selected from the group consisting of hydrogen, halo, C₁₋₆alkyl, C₃₋₆cycloalkyl, CN, CF₃, CHF₂, OCHF₂ and OCF₃, with at least one of R^{1a}, R^{1b}, and R^{1c} not being hydrogen.

In a preferable embodiment, R^{1a} is halo, R^{1b} is selected from the group consisting of halo, CF₃, cyano, OCHF₂ and OCF₃, and R^{1c} is hydrogen. In another preferable embodiment, R^{1a} is halo, R^{1b} is selected from the group consisting of halo, CF₃ and cyano, and R^{1c} is hydrogen. In a further embodiment, R^{1a} is halo, R^{1b} is halo and R^{1c} is hydrogen. In yet another embodiment, R^{1a} is halo, R^{1b} is CN, and R^{1c} is hydrogen.

Preferably, the halo is C₁.

In an embodiment, R² is selected from the group consisting of CHF₂, CF₃, C₁₋₆alkyl, C₁₋₆alkylOC₁-6alkyl, C₃₋₆cycloalkyl and CON(R_{S})₂; and the the structure of Formula (I) has Formula (I-1) or Formula (I-2), R_{S} is each independently selected from the group consisting of H, C₁₋₆alkyl, CN, SOC₁₋₆alkyl, SO₂C₁₋₆alkyl, SO₂OH, C₃₋₆cycloalkyl,

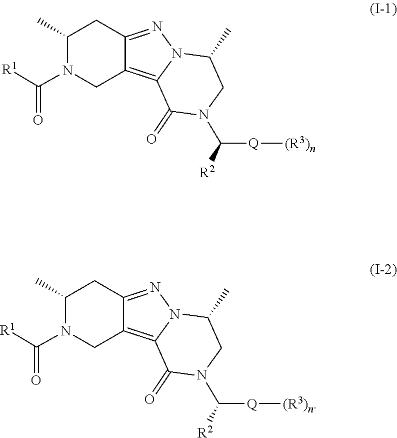

In an embodiment, R_{S} is each independently selected from the group consisting of H, and C₁₋₆alkyl.

In a specific embodiment, R² is selected from the group consisting of C₁₋₆alkyl and CON(R_{S})₂. Preferably, R² is methyl or CONHCH₃.

In an embodiment, Q represents a ring selected from the group consisting of phenyl and 6-membered heteroaryl. Preferably, Q is a ring of 6-membered heteroaryl, which is selected from the group consisting of pyridyl, pyrimidinyl, pyridazinyl and pyrazinyl.

In another embodiment, Q represents a ring selected from the group consisting of 5-membered heteroaryl. In a specific embodiment, Q is a ring selected from the group consisting of oxadiazolyl, pyrazolyl and imidazolyl, preferably pyrazolyl or oxadiazolyl.

In an embodiment, the structural unit

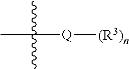

in Formula (I) of the present disclosure satisfies Formula (Ib)

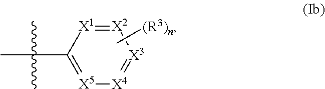

wherein,

all of X¹, X², X³, X⁴ and X⁵ are CH.

In the embodiment where all of X¹, X², X³, X⁴ and X⁵ are CH, the ring is a phenyl ring.

In such an embodiment, R³(s) is/are each independently linked to n of X¹-X⁵. Preferably, n is 1 or 2, and R³(s) is/are linked to one or two of X¹-X⁵. In a specific embodiment where n is 1, R³ is linked to X³. In an alternative embodiment where n is 2, R³s are linked to two of X¹-X⁵. In an exemplary embodiment where n is 2, one R³ is linked to X³ and other is linked to X¹ or X². It should be noted that when there are more than one R³, they are independently selected and thus may be identical or different.

In an embodiment, the structural unit

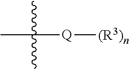

in Formula (I) of the present disclosure satisfies Formula (Ib)

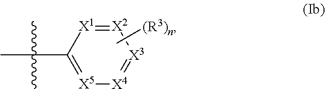

wherein,

one or two of X¹, X², X³, X⁴ and X⁵ are N, and rest of them are CH.

In the embodiment where one or two of X¹, X², X³, X⁴ and X⁵ are N, and rest of them are CH, the ring is a 6-membered heteroaryl. In such an embodiment, R³(s) are each independently linked to n of X¹-X⁵, which are CH. Preferably, n is 1 or 2 and R³(s) is/are linked to one or two of X¹-X⁵ which is/are CH. In a specific embodiment where n is 1, R³ is linked to X³ which is CH. In an alternative embodiment where n is 2, R³s are linked to two of X¹-X⁵. In an exemplary embodiment where n is 2, one R³ is linked to X³ (which is CH) and other is linked to X¹ (which is CH), X² (which is CH), X⁴ (which is CH) or X⁵ (which is CH). It should be noted that when there are more than one R³, they are independently selected and thus may be identical or different.

In a specific embodiment, one of X¹-X⁵ is N, and rest of them are CH. In another specific embodiment, two of X¹-X⁵ are N, and rest of them are CH.

In a particular embodiment,

X¹ is N, and X², X³, X⁴, X⁵ are CH; or

X² is N, and X¹, X³, X⁴, X⁵ are CH; or

X³ is N, and X¹, X², X⁴, X⁵ are CH; or

X¹ and X² are N, and X³, X⁴, X⁵ are CH; or

X¹ and X³ are N, and X², X⁴, X⁵ are CH; or

X¹ and X⁴ are N, and X², X³, X⁵ are CH; or

X¹ and X⁵ are N, and X², X³, X⁴ are CH; or

X² and X³ are N, and X¹, X⁴, X⁵ are CH; or

X² and X⁴ are N, and X¹, X³, X⁵ are CH.

In another particular embodiment,

both of X¹ and X² are N, and X⁴ and X⁵ are CH; or

both of X² and X⁴ are N, and X¹ and X⁵ are CH; or

both of X¹ and X⁴ are N, and X² and X⁵ are CH; or

both of X¹ and X⁵ are N, and X² and X⁴ are CH.

In a specific embodiment, R³s is/are linked to one or two of X¹-X⁵, which is/are CH.

In an embodiment, the structural unit

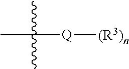

in Formula (I) of the present disclosure satisfies Formula (Ic)

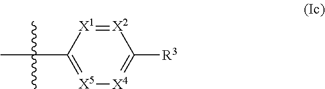

wherein

all of X¹, X², X⁴ and X⁵ are CH.

In a specific embodiment, one or two of X¹, X², X⁴, X⁵ is/are optionally substituted by another R³(s). It should be noted that when there are more than one R³, they are independently selected and thus may be identical or different.

In an embodiment, the structural unit

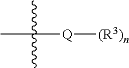

in Formula (I) of the present disclosure satisfies Formula (Ic)

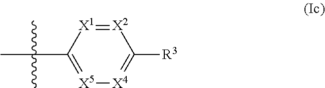

wherein

one of X² and X⁴ is N, and the other is CH.

In a specific embodiment of the above Formula (Ic),
i) one of X¹ and X⁵ is N, and rest of them is CH; or
ii) both of X¹ and X⁵ are CH.

In a specific embodiment, one or two of X¹, X², X⁴, X⁵ (which is/are CH) is/are optionally substituted by another R³(s). It should be noted that when there are more than one R³, they are independently selected and thus may be identical or different.

In an embodiment, the structural unit

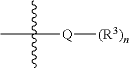

in Formula (I) of the present disclosure satisfies Formula (Ic)

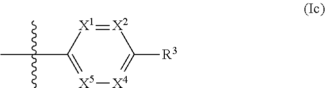

wherein

both of X² and X⁴ are N.

In a specific embodiment of the above Formula (Ic),

both of X¹ and X⁵ are CH.

In a specific embodiment, one or two of X¹, X⁵ (which is/are CH) is/are optionally substituted by another R³(s). It should be noted that when there are more than one R³, they are independently selected and thus may be identical or different.

In an embodiment, the structural unit

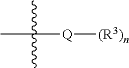

n in Formula (I) of the present disclosure satisfies Formula (Ic)

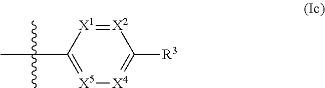

wherein

one of X¹ and X⁵ is N, and the other is CH.

In a specific embodiment of the above Formula (Ic),
i) one of X² and X⁴ is N, and rest of them is CH; or
ii) both of X² and X⁴ are CH.

In a specific embodiment, one or two of X¹, X², X⁴, X⁵ (which is/are CH) is/are optionally substituted by another R³(s). It should be noted that when there are more than one R³, they are independently selected and thus may be identical or different.

In an embodiment, the structural unit

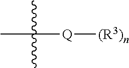

in Formula (I) of the present disclosure satisfies Formula (Ic)

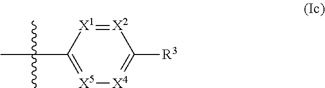

wherein

both of X¹ and X⁵ are N.

In a specific embodiment of the above Formula (Ic),

both of X² and X⁴ are CH.

In a specific embodiment, one or two of X², X⁴ (which is/are CH) is/are optionally substituted by another R³(s). It should be noted that when there are more than one R³, they are independently selected and thus may be identical or different.

In an embodiment, the structural unit

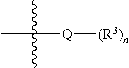

in Formula (I) satisfies Formula (Ic)

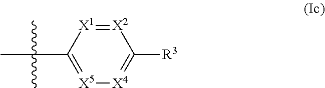

wherein

all of X¹, X², X⁴ and X⁵ are CH; or

X² is N, and X¹, X⁴ and X⁵ are CH; or

X¹ is N, and X², X⁴ and X⁵ are CH.

In a specific embodiment, the structural unit

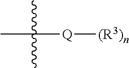

in Formula (I) satisfies Formula (Ic)

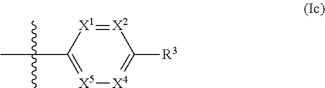

both of X¹ and X² are N, and X⁴ and X⁵ are CH; or

both of X² and X⁴ are N, and X¹ and X⁵ are CH; or

both of X¹ and X⁴ are N, and X² and X⁵ are CH; or

both of X¹ and X⁵ are N, and X² and X⁴ are CH.

In an embodiment, the structural unit

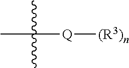

in Formula (I) of the present disclosure satisfies Formula (Ib′)

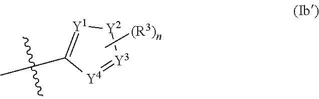

wherein one, two or three of Y¹, Y², Y³ and Y⁴ are N or NH or O, and rest of them are CH. In another embodiment, one or two of Y¹, Y², Y³ and Y⁴ are N (or NH), and rest of them are CH.

In a preferable embodiment, two of Y¹, Y², Y³ and Y⁴ are N (or NH), and rest of them are CH. More preferably, Y¹ and Y² are N (or NH).

In a preferable embodiment, Y¹ and Y² are N or NH, and Y³ and Y⁴ are CH.

In another preferable embodiment, Y¹ and Y² are N or NH, Y⁴ is O, and Y³ is CH.

In the embodiment where one, two or three of Y¹, Y², Y³ and Y⁴ are N (or NH) or O, and rest of them are CH, the ring is a 5-membered heteroaryl. In such an embodiment, R³(s) are each independently linked to one or more of Y¹-Y⁴. Preferably, n is 1 or 2 and R³(s) is/are linked to one or two of Y¹-Y⁴.

In a preferable embodiment where n is 1, R³ is linked to Y². In a preferable embodiment where n is 1, R³ is linked to Y¹. In a preferable embodiment where n is 1, R³ is linked to Y³. More preferably, Y¹ and Y² are N (or NH); or Y¹ or Y² is N (or NH). In another embodiment, Y⁴ is O.

In a specific embodiment, Q is a ring of

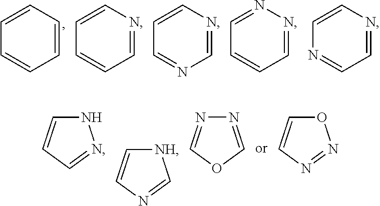

(where H may be absence due to substitution or connection to other part of the molecule), which is substituted with n R³.

In an embodiment, the CH or NH moiety as described may be optionally substituted, for example, by R³.

It should be noted that when a CH or NH moiety is present, for example within a ring like phenyl or heteroaryl, the “H” may be absent due to substitution or connection to other part of the molecule. Similarly, when a C or N moiety is mentioned, for example within a ring like phenyl or heteroaryl, a H could be present such that a stable compound structure is satisfied, and the corresponding atom or radical may be described only as C or N. For example, in the Formulae like (I), (I-1), (I-2), (Ib), (Ib′), (Ic) etc, when X (e.g., X¹, X², X³, X⁴, X⁵) is described as CH or NH, it means the group is unsubstituted or optionally substituted or connected to other part of the molecule, provided that a stable compound is achieved. Likewise, when C or N is described, “H” or optional substitution/connection may be added such that a stable compound is satisfied.

In an embodiment, n is 0, 1 or 2, e.g., 1 or 2. It should be noted that when there are more than one R³, they are independently selected and thus may be identical or different. When n is 0, it means Q is not substituted or the substituent is H.

In an embodiment, halo is F, Cl or Br, preferably F or Cl.

In an embodiment, R³ independently represents a substituent selected from the group consisting of CN, halo, and oxo.

In an embodiment, R³ independently represents a substituent selected from the group consisting of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SO₂C₁₋₆alkyl, SO₂N(R_{S})₂, SO(C₁₋₆alkyl)NRS, CON(R_{S})₂, and N(R_{S})₂. Each of these groups is optionally substituted with 1, 2, 3, 4 or 5 substituents, each of said substituents independently selected from the group consisting of halo, hydroxyl, C₁₋₆alkyl and oxo.

In another embodiment, R³ independently represents a substituent selected from the group consisting of C₁₋₆alkyl, C₁₋₆alkoxyl, C₃₋₆cycloalkyl, 5-membered heteroaryl and 4-8 membered heterocyclyl. Each of C₁₋₆alkyl, C₁₋₆alkoxyl, C₃₋₆cycloalkyl, 5-membered heteroaryl and 4-8 membered heterocyclyl is optionally substituted with 1, 2, 3, 4 or 5 substituents, each of said substituents independently selected from the group consisting of halo, hydroxyl, C₁₋₆alkyl and oxo.

Preferably, the number of substitutes on any of C₁₋₆alkyl, C₁₋₆alkoxyl, C₃₋₆cycloalkyl, 5-membered heteroaryl, 6-membered heteroaryl, 4-8 membered heterocyclyl, O—C₃-6cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂) is 1, 2 or 3.

In a specific embodiment, R³ is independently selected from the group consisting of OCHF₂, CHF₂, CH₂CF₃, C(CH₃)₂OH, CH₂C(CH₃)₂OH, cyclopropyl, CH₃, CF₃,

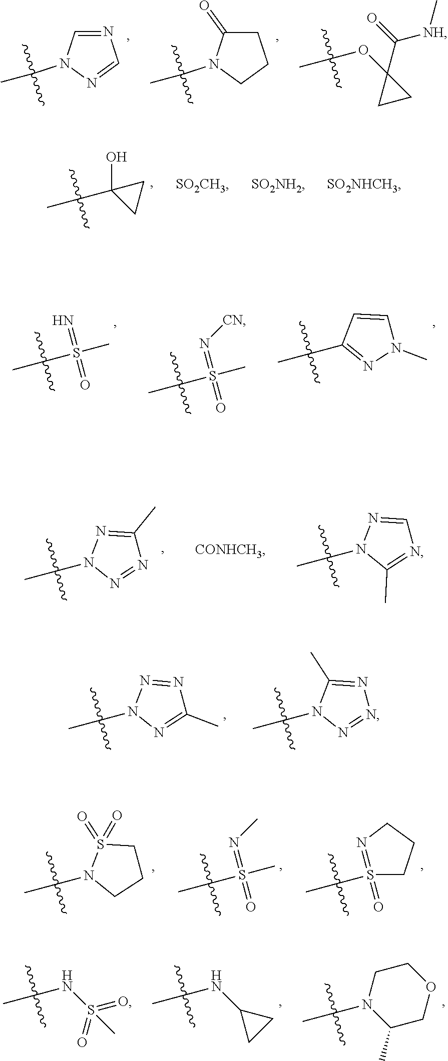

halo, oxo, isopropyl, O-isopropyl, and CN.

In another specific embodiment, R³ is independently selected from the group consisting of OCHF₂, C(CH₃)₂OH, cyclopropyl, CH₃, CF₃,

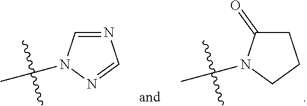 [SMILES: *n1cncn1 ; *N1CCCC1=O]

In an embodiment, R_{S} is each independently selected from the group consisting of H, C₁₋₆alkyl, CN, SOC₁₋₆alkyl, SO₂C₁₋₆alkyl, SO₂OH, C₃₋₆cycloalkyl.

In a preferable embodiment, R_{S} is each independently selected from the group consisting of H, C₁₋₆alkyl, SO₂C₁₋₆alkyl, C₃₋₆cycloalkyl.

In an embodiment, the heteroaryl (such as, 5-membered heteroaryl or 6-membered heteroaryl) may contain at least one (e.g., 1, 2 or 3, preferably 1 or 2) heteroatoms independently selected from the group consisting of N, O and S, preferably N and O.

In an embodiment, the heterocyclyl (such as, 4-8 membered heterocyclyl) may contain at least one (e.g., 1, 2 or 3, preferably 1 or 2) heteroatoms independently selected from the group consisting of N, O and S, preferably N and O.

A further embodiment of the present disclosure is a compound selected from the group consisting of the compounds described in Table 1 and Table 2 below, a stereoisomer or tautomer thereof, or a pharmaceutically acceptable salt thereof.

TABLE 1

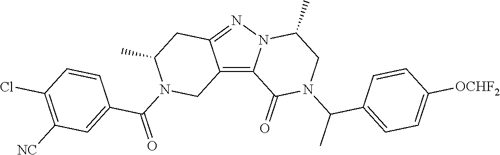 [SMILES: CC(c1ccc(OC(F)F)cc1)N1C[C@@H](C)n2nc3c(c2C1=O)CN(C(=O)c1ccc(Cl)c(C#N)c1)[C@H](C)C3]

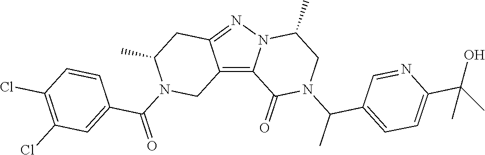 [SMILES: CC(c1ccc(C(C)(C)O)nc1)N1C[C@@H](C)n2nc3c(c2C1=O)CN(C(=O)c1ccc(Cl)c(Cl)c1)[C@H](C)C3]

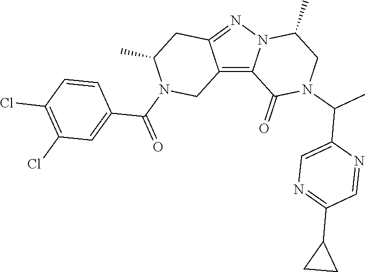 [SMILES: CC(c1cnc(C2CC2)cn1)N1C[C@@H](C)n2nc3c(c2C1=O)CN(C(=O)c1ccc(Cl)c(Cl)c1)[C@H](C)C3]

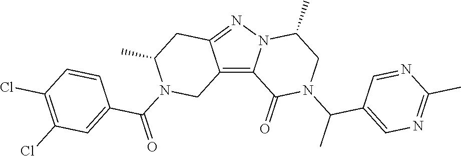 [SMILES: Cc1ncc(C(C)N2C[C@@H](C)n3nc4c(c3C2=O)CN(C(=O)c2ccc(Cl)c(Cl)c2)[C@H](C)C4)cn1]

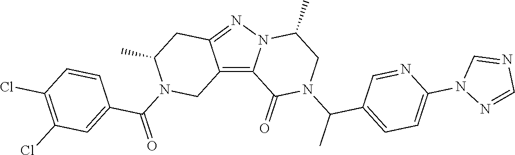 [SMILES: CC(c1ccc(-n2cncn2)nc1)N1C[C@@H](C)n2nc3c(c2C1=O)CN(C(=O)c1ccc(Cl)c(Cl)c1)[C@H](C)C3]

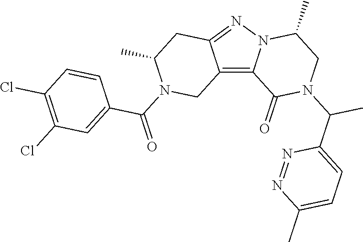 [SMILES: Cc1ccc(C(C)N2C[C@@H](C)n3nc4c(c3C2=O)CN(C(=O)c2ccc(Cl)c(Cl)c2)[C@H](C)C4)nn1]

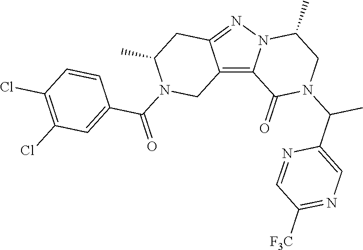 [SMILES: CC(c1cnc(C(F)(F)F)cn1)N1C[C@@H](C)n2nc3c(c2C1=O)CN(C(=O)c1ccc(Cl)c(Cl)c1)[C@H](C)C3]

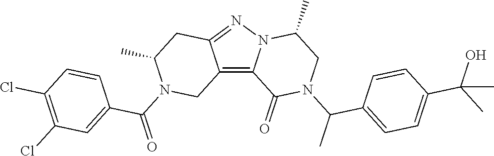 [SMILES: CC(c1ccc(C(C)(C)O)cc1)N1C[C@@H](C)n2nc3c(c2C1=O)CN(C(=O)c1ccc(Cl)c(Cl)c1)[C@H](C)C3]

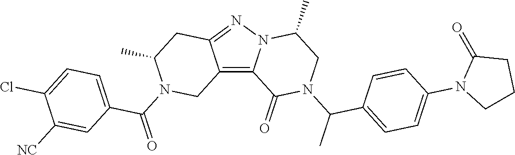 [SMILES: CC(c1ccc(N2CCCC2=O)cc1)N1C[C@@H](C)n2nc3c(c2C1=O)CN(C(=O)c1ccc(Cl)c(C#N)c1)[C@H](C)C3]

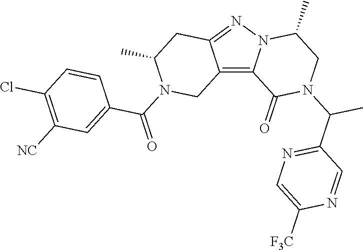 [SMILES: CC(c1cnc(C(F)(F)F)cn1)N1C[C@@H](C)n2nc3c(c2C1=O)CN(C(=O)c1ccc(Cl)c(C#N)c1)[C@H](C)C3]

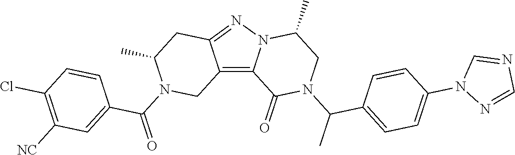 [SMILES: CC(c1ccc(-n2cncn2)cc1)N1C[C@@H](C)n2nc3c(c2C1=O)CN(C(=O)c1ccc(Cl)c(C#N)c1)[C@H](C)C3]

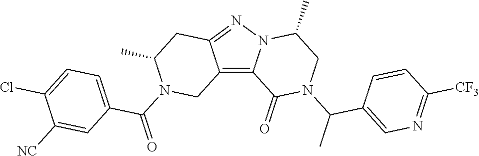 [SMILES: CC(c1ccc(C(F)(F)F)nc1)N1C[C@@H](C)n2nc3c(c2C1=O)CN(C(=O)c1ccc(Cl)c(C#N)c1)[C@H](C)C3]

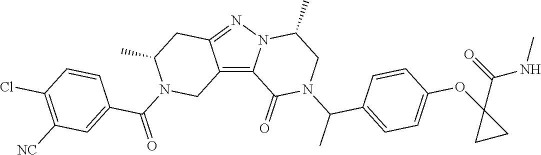 [SMILES: CNC(=O)C1(Oc2ccc(C(C)N3C[C@@H](C)n4nc5c(c4C3=O)CN(C(=O)c3ccc(Cl)c(C#N)c3)[C@H](C)C5)cc2)CC1]

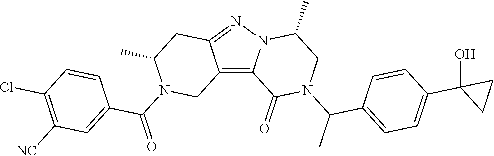 [SMILES: CC(c1ccc(C2(O)CC2)cc1)N1C[C@@H](C)n2nc3c(c2C1=O)CN(C(=O)c1ccc(Cl)c(C#N)c1)[C@H](C)C3]

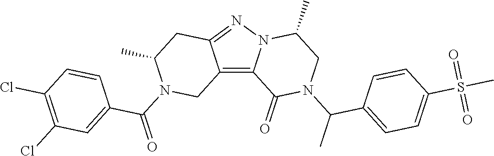 [SMILES: CC(c1ccc(S(C)(=O)=O)cc1)N1C[C@@H](C)n2nc3c(c2C1=O)CN(C(=O)c1ccc(Cl)c(Cl)c1)[C@H](C)C3]

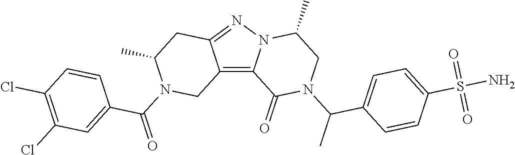 [SMILES: CC(c1ccc(S(N)(=O)=O)cc1)N1C[C@@H](C)n2nc3c(c2C1=O)CN(C(=O)c1ccc(Cl)c(Cl)c1)[C@H](C)C3]

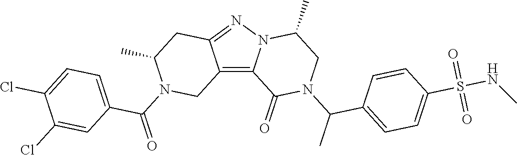 [SMILES: CNS(=O)(=O)c1ccc(C(C)N2C[C@@H](C)n3nc4c(c3C2=O)CN(C(=O)c2ccc(Cl)c(Cl)c2)[C@H](C)C4)cc1]

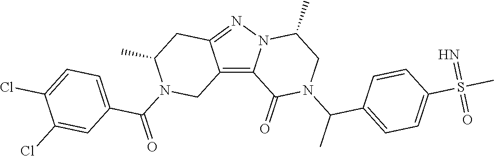 [SMILES: CC(c1ccc(S(C)(=N)=O)cc1)N1C[C@@H](C)n2nc3c(c2C1=O)CN(C(=O)c1ccc(Cl)c(Cl)c1)[C@H](C)C3]

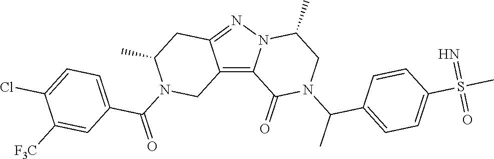 [SMILES: CC(c1ccc(S(C)(=N)=O)cc1)N1C[C@@H](C)n2nc3c(c2C1=O)CN(C(=O)c1ccc(Cl)c(C(F)(F)F)c1)[C@H](C)C3]

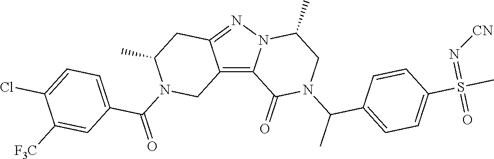 [SMILES: CC(c1ccc(S(C)(=O)=NC#N)cc1)N1C[C@@H](C)n2nc3c(c2C1=O)CN(C(=O)c1ccc(Cl)c(C(F)(F)F)c1)[C@H](C)C3]

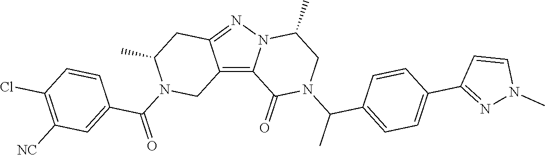 [SMILES: CC(c1ccc(-c2ccn(C)n2)cc1)N1C[C@@H](C)n2nc3c(c2C1=O)CN(C(=O)c1ccc(Cl)c(C#N)c1)[C@H](C)C3]

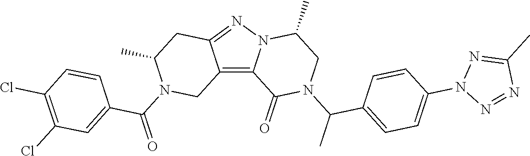 [SMILES: Cc1nnn(-c2ccc(C(C)N3C[C@@H](C)n4nc5c(c4C3=O)CN(C(=O)c3ccc(Cl)c(Cl)c3)[C@H](C)C5)cc2)n1]

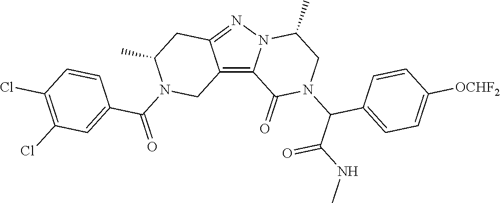 [SMILES: CNC(=O)C(c1ccc(OC(F)F)cc1)N1C[C@@H](C)n2nc3c(c2C1=O)CN(C(=O)c1ccc(Cl)c(Cl)c1)[C@H](C)C3]

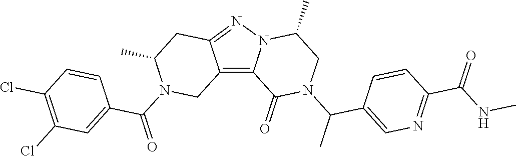 [SMILES: CNC(=O)c1ccc(C(C)N2C[C@@H](C)n3nc4c(c3C2=O)CN(C(=O)c2ccc(Cl)c(Cl)c2)[C@H](C)C4)cn1]

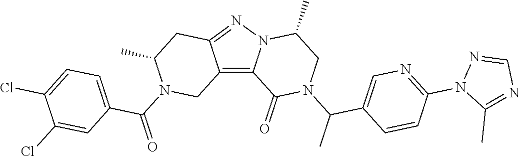 [SMILES: Cc1ncnn1-c1ccc(C(C)N2C[C@@H](C)n3nc4c(c3C2=O)CN(C(=O)c2ccc(Cl)c(Cl)c2)[C@H](C)C4)cn1]

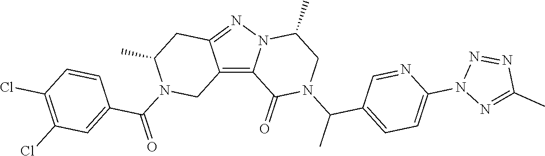 [SMILES: Cc1nnn(-c2ccc(C(C)N3C[C@@H](C)n4nc5c(c4C3=O)CN(C(=O)c3ccc(Cl)c(Cl)c3)[C@H](C)C5)cn2)n1]

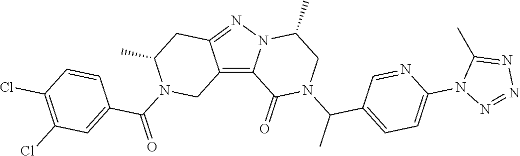 [SMILES: Cc1nnnn1-c1ccc(C(C)N2C[C@@H](C)n3nc4c(c3C2=O)CN(C(=O)c2ccc(Cl)c(Cl)c2)[C@H](C)C4)cn1]

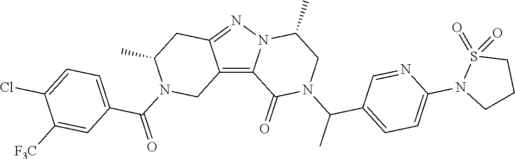 [SMILES: CC(c1ccc(N2CCCS2(=O)=O)nc1)N1C[C@@H](C)n2nc3c(c2C1=O)CN(C(=O)c1ccc(Cl)c(C(F)(F)F)c1)[C@H](C)C3]

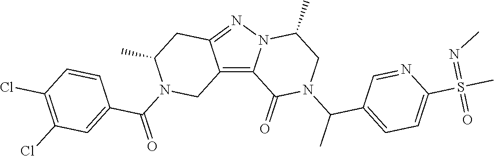 [SMILES: CN=S(C)(=O)c1ccc(C(C)N2C[C@@H](C)n3nc4c(c3C2=O)CN(C(=O)c2ccc(Cl)c(Cl)c2)[C@H](C)C4)cn1]

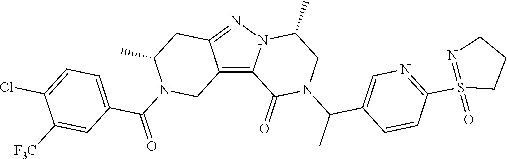 [SMILES: CC(c1ccc(S2(=O)=NCCC2)nc1)N1C[C@@H](C)n2nc3c(c2C1=O)CN(C(=O)c1ccc(Cl)c(C(F)(F)F)c1)[C@H](C)C3]

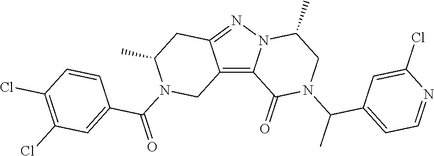 [SMILES: CC(c1ccnc(Cl)c1)N1C[C@@H](C)n2nc3c(c2C1=O)CN(C(=O)c1ccc(Cl)c(Cl)c1)[C@H](C)C3]

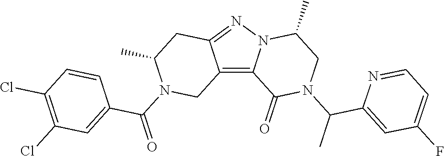 [SMILES: CC(c1cc(F)ccn1)N1C[C@@H](C)n2nc3c(c2C1=O)CN(C(=O)c1ccc(Cl)c(Cl)c1)[C@H](C)C3]

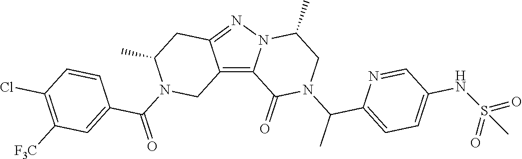 [SMILES: CC(c1ccc(NS(C)(=O)=O)cn1)N1C[C@@H](C)n2nc3c(c2C1=O)CN(C(=O)c1ccc(Cl)c(C(F)(F)F)c1)[C@H](C)C3]

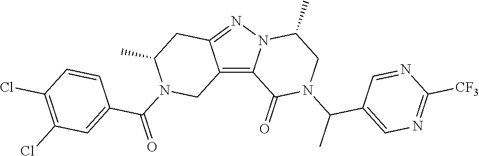 [SMILES: CC(c1cnc(C(F)(F)F)nc1)N1C[C@@H](C)n2nc3c(c2C1=O)CN(C(=O)c1ccc(Cl)c(Cl)c1)[C@H](C)C3]

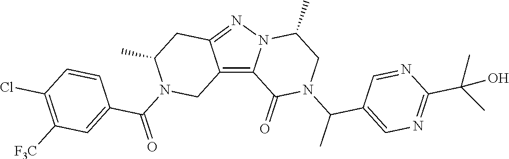 [SMILES: CC(c1cnc(C(C)(C)O)nc1)N1C[C@@H](C)n2nc3c(c2C1=O)CN(C(=O)c1ccc(Cl)c(C(F)(F)F)c1)[C@H](C)C3]

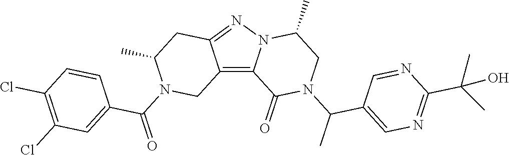 [SMILES: CC(c1cnc(C(C)(C)O)nc1)N1C[C@@H](C)n2nc3c(c2C1=O)CN(C(=O)c1ccc(Cl)c(Cl)c1)[C@H](C)C3]

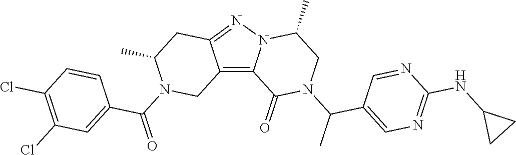 [SMILES: CC(c1cnc(NC2CC2)nc1)N1C[C@@H](C)n2nc3c(c2C1=O)CN(C(=O)c1ccc(Cl)c(Cl)c1)[C@H](C)C3]

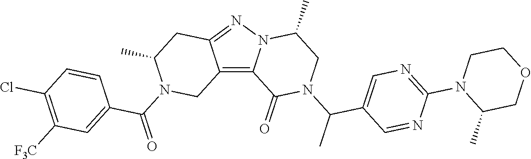 [SMILES: CC(c1cnc(N2CCOC[C@@H]2C)nc1)N1C[C@@H](C)n2nc3c(c2C1=O)CN(C(=O)c1ccc(Cl)c(C(F)(F)F)c1)[C@H](C)C3]

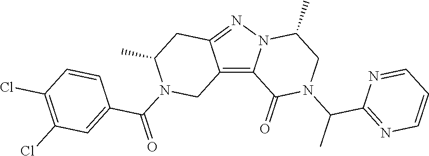 [SMILES: CC(c1ncccn1)N1C[C@@H](C)n2nc3c(c2C1=O)CN(C(=O)c1ccc(Cl)c(Cl)c1)[C@H](C)C3]

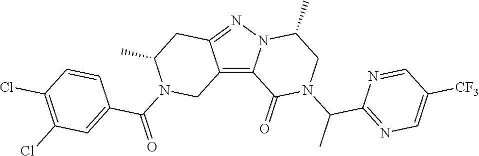 [SMILES: CC(c1ncc(C(F)(F)F)cn1)N1C[C@@H](C)n2nc3c(c2C1=O)CN(C(=O)c1ccc(Cl)c(Cl)c1)[C@H](C)C3]

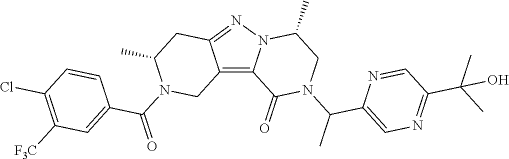 [SMILES: CC(c1cnc(C(C)(C)O)cn1)N1C[C@@H](C)n2nc3c(c2C1=O)CN(C(=O)c1ccc(Cl)c(C(F)(F)F)c1)[C@H](C)C3]

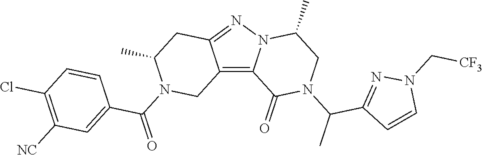 [SMILES: CC(c1ccn(CC(F)(F)F)n1)N1C[C@@H](C)n2nc3c(c2C1=O)CN(C(=O)c1ccc(Cl)c(C#N)c1)[C@H](C)C3]

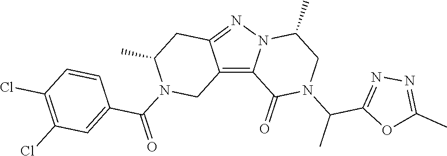 [SMILES: Cc1nnc(C(C)N2C[C@@H](C)n3nc4c(c3C2=O)CN(C(=O)c2ccc(Cl)c(Cl)c2)[C@H](C)C4)o1]

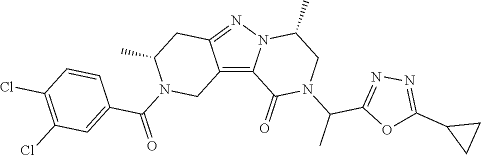 [SMILES: CC(c1nnc(C2CC2)o1)N1C[C@@H](C)n2nc3c(c2C1=O)CN(C(=O)c1ccc(Cl)c(Cl)c1)[C@H](C)C3]

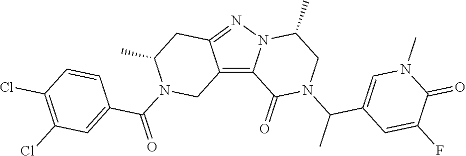 [SMILES: CC(c1cc(F)c(=O)n(C)c1)N1C[C@@H](C)n2nc3c(c2C1=O)CN(C(=O)c1ccc(Cl)c(Cl)c1)[C@H](C)C3]

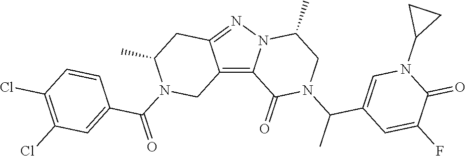 [SMILES: CC(c1cc(F)c(=O)n(C2CC2)c1)N1C[C@@H](C)n2nc3c(c2C1=O)CN(C(=O)c1ccc(Cl)c(Cl)c1)[C@H](C)C3]

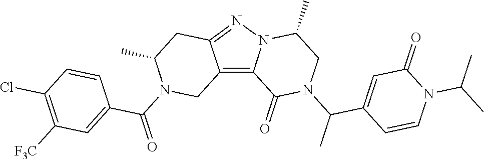 [SMILES: CC(c1ccn(C(C)C)c(=O)c1)N1C[C@@H](C)n2nc3c(c2C1=O)CN(C(=O)c1ccc(Cl)c(C(F)(F)F)c1)[C@H](C)C3]

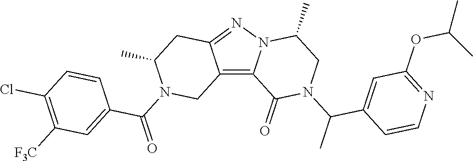 [SMILES: CC(C)Oc1cc(C(C)N2C[C@@H](C)n3nc4c(c3C2=O)CN(C(=O)c2ccc(Cl)c(C(F)(F)F)c2)[C@H](C)C4)ccn1]

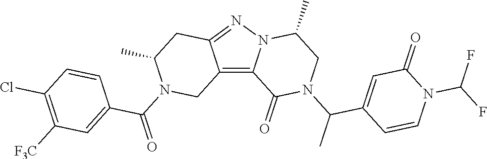 [SMILES: CC(c1ccn(C(F)F)c(=O)c1)N1C[C@@H](C)n2nc3c(c2C1=O)CN(C(=O)c1ccc(Cl)c(C(F)(F)F)c1)[C@H](C)C3]

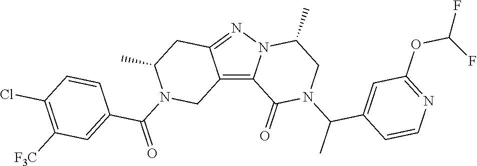 [SMILES: CC(c1ccnc(OC(F)F)c1)N1C[C@@H](C)n2nc3c(c2C1=O)CN(C(=O)c1ccc(Cl)c(C(F)(F)F)c1)[C@H](C)C3]

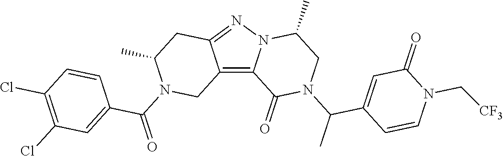 [SMILES: CC(c1ccn(CC(F)(F)F)c(=O)c1)N1C[C@@H](C)n2nc3c(c2C1=O)CN(C(=O)c1ccc(Cl)c(Cl)c1)[C@H](C)C3]

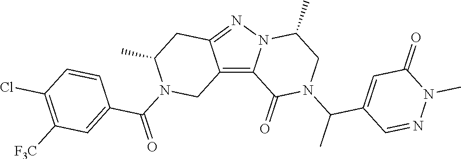 [SMILES: CC(c1cnn(C)c(=O)c1)N1C[C@@H](C)n2nc3c(c2C1=O)CN(C(=O)c1ccc(Cl)c(C(F)(F)F)c1)[C@H](C)C3]

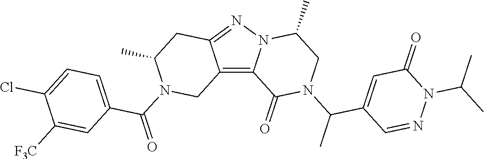 [SMILES: CC(c1cnn(C(C)C)c(=O)c1)N1C[C@@H](C)n2nc3c(c2C1=O)CN(C(=O)c1ccc(Cl)c(C(F)(F)F)c1)[C@H](C)C3]

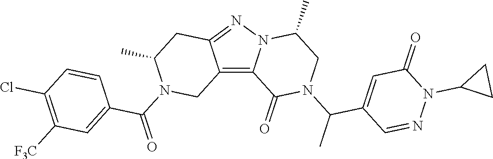 [SMILES: CC(c1cnn(C2CC2)c(=O)c1)N1C[C@@H](C)n2nc3c(c2C1=O)CN(C(=O)c1ccc(Cl)c(C(F)(F)F)c1)[C@H](C)C3]

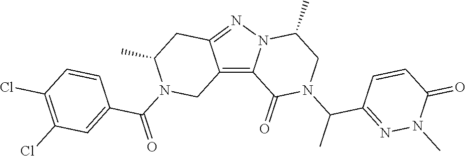 [SMILES: CC(c1ccc(=O)n(C)n1)N1C[C@@H](C)n2nc3c(c2C1=O)CN(C(=O)c1ccc(Cl)c(Cl)c1)[C@H](C)C3]

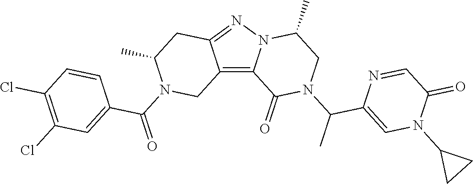 [SMILES: CC(c1cn(C2CC2)c(=O)cn1)N1C[C@@H](C)n2nc3c(c2C1=O)CN(C(=O)c1ccc(Cl)c(Cl)c1)[C@H](C)C3]

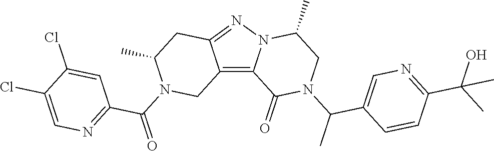 [SMILES: CC(c1ccc(C(C)(C)O)nc1)N1C[C@@H](C)n2nc3c(c2C1=O)CN(C(=O)c1cc(Cl)c(Cl)cn1)[C@H](C)C3]

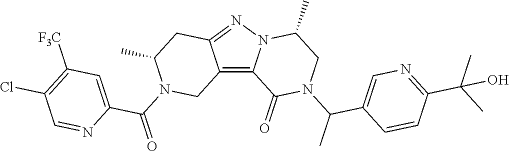 [SMILES: CC(c1ccc(C(C)(C)O)nc1)N1C[C@@H](C)n2nc3c(c2C1=O)CN(C(=O)c1cc(C(F)(F)F)c(Cl)cn1)[C@H](C)C3]

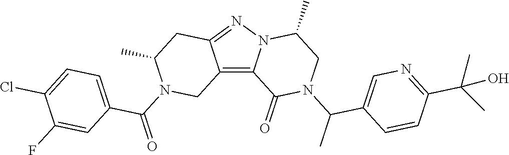 [SMILES: CC(c1ccc(C(C)(C)O)nc1)N1C[C@@H](C)n2nc3c(c2C1=O)CN(C(=O)c1ccc(Cl)c(F)c1)[C@H](C)C3]

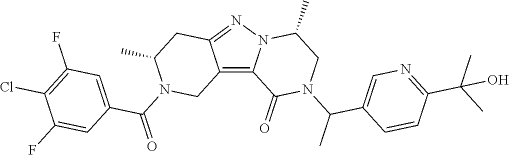 [SMILES: CC(c1ccc(C(C)(C)O)nc1)N1C[C@@H](C)n2nc3c(c2C1=O)CN(C(=O)c1cc(F)c(Cl)c(F)c1)[C@H](C)C3]

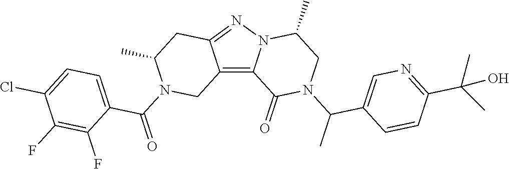 [SMILES: CC(c1ccc(C(C)(C)O)nc1)N1C[C@@H](C)n2nc3c(c2C1=O)CN(C(=O)c1ccc(Cl)c(F)c1F)[C@H](C)C3]

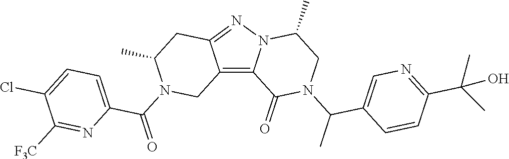 [SMILES: CC(c1ccc(C(C)(C)O)nc1)N1C[C@@H](C)n2nc3c(c2C1=O)CN(C(=O)c1ccc(Cl)c(C(F)(F)F)n1)[C@H](C)C3]

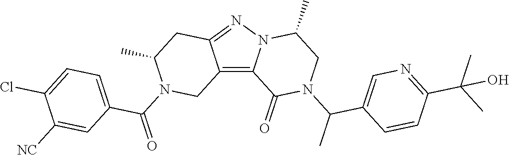 [SMILES: CC(c1ccc(C(C)(C)O)nc1)N1C[C@@H](C)n2nc3c(c2C1=O)CN(C(=O)c1ccc(Cl)c(C#N)c1)[C@H](C)C3]

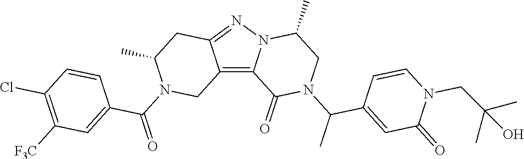 [SMILES: CC(c1ccn(CC(C)(C)O)c(=O)c1)N1C[C@@H](C)n2nc3c(c2C1=O)CN(C(=O)c1ccc(Cl)c(C(F)(F)F)c1)[C@H](C)C3]

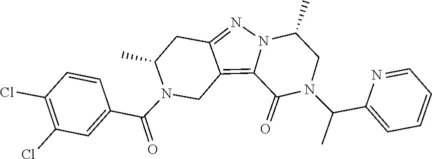 [SMILES: CC(c1ccccn1)N1C[C@@H](C)n2nc3c(c2C1=O)CN(C(=O)c1ccc(Cl)c(Cl)c1)[C@H](C)C3]

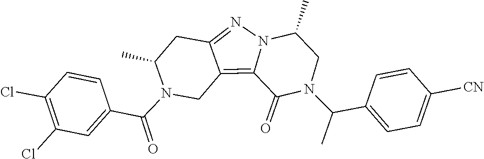 [SMILES: CC(c1ccc(C#N)cc1)N1C[C@@H](C)n2nc3c(c2C1=O)CN(C(=O)c1ccc(Cl)c(Cl)c1)[C@H](C)C3]

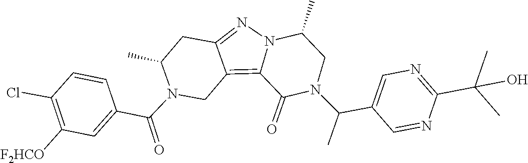 [SMILES: CC(c1cnc(C(C)(C)O)nc1)N1C[C@@H](C)n2nc3c(c2C1=O)CN(C(=O)c1ccc(Cl)c(OC(F)F)c1)[C@H](C)C3]

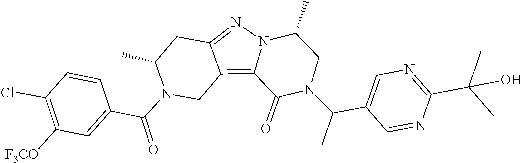 [SMILES: CC(c1cnc(C(C)(C)O)nc1)N1C[C@@H](C)n2nc3c(c2C1=O)CN(C(=O)c1ccc(Cl)c(OC(F)(F)F)c1)[C@H](C)C3]

TABLE 2

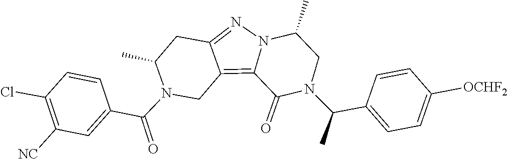 [SMILES: C[C@@H]1Cc2nn3c(c2CN1C(=O)c1ccc(Cl)c(C#N)c1)C(=O)N([C@H](C)c1ccc(OC(F)F)cc1)C[C@H]3C]

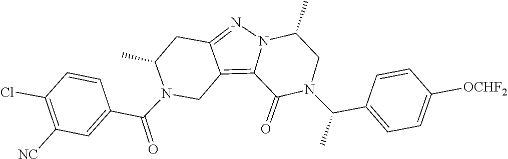 [SMILES: C[C@@H]1Cc2nn3c(c2CN1C(=O)c1ccc(Cl)c(C#N)c1)C(=O)N([C@@H](C)c1ccc(OC(F)F)cc1)C[C@H]3C]

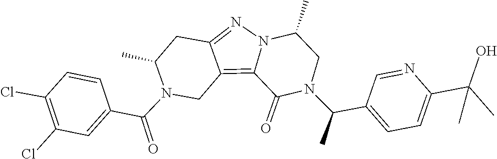 [SMILES: C[C@@H]1Cc2nn3c(c2CN1C(=O)c1ccc(Cl)c(Cl)c1)C(=O)N([C@H](C)c1ccc(C(C)(C)O)nc1)C[C@H]3C]

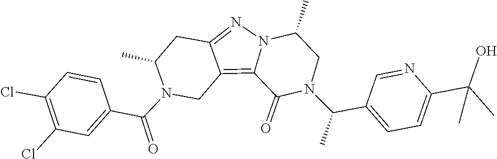 [SMILES: C[C@@H]1Cc2nn3c(c2CN1C(=O)c1ccc(Cl)c(Cl)c1)C(=O)N([C@@H](C)c1ccc(C(C)(C)O)nc1)C[C@H]3C]

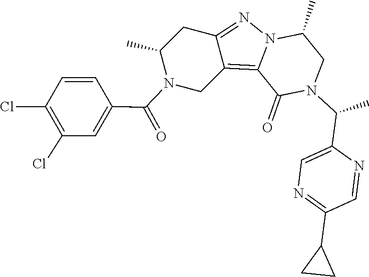 [SMILES: C[C@@H]1Cc2nn3c(c2CN1C(=O)c1ccc(Cl)c(Cl)c1)C(=O)N([C@H](C)c1cnc(C2CC2)cn1)C[C@H]3C]

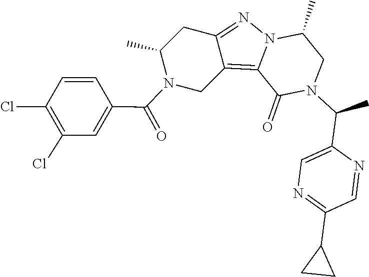 [SMILES: C[C@@H]1Cc2nn3c(c2CN1C(=O)c1ccc(Cl)c(Cl)c1)C(=O)N([C@@H](C)c1cnc(C2CC2)cn1)C[C@H]3C]

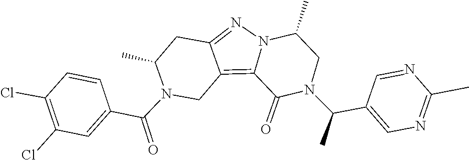 [SMILES: Cc1ncc([C@@H](C)N2C[C@@H](C)n3nc4c(c3C2=O)CN(C(=O)c2ccc(Cl)c(Cl)c2)[C@H](C)C4)cn1]

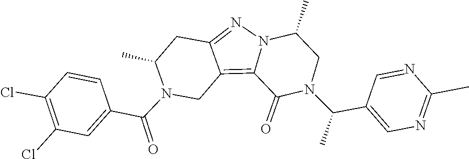 [SMILES: Cc1ncc([C@H](C)N2C[C@@H](C)n3nc4c(c3C2=O)CN(C(=O)c2ccc(Cl)c(Cl)c2)[C@H](C)C4)cn1]

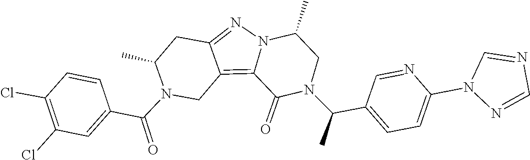 [SMILES: C[C@@H]1Cc2nn3c(c2CN1C(=O)c1ccc(Cl)c(Cl)c1)C(=O)N([C@H](C)c1ccc(-n2cncn2)nc1)C[C@H]3C]

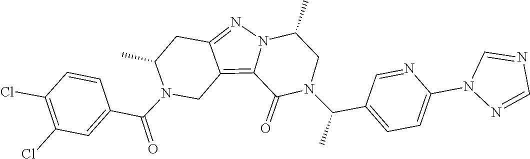 [SMILES: C[C@@H]1Cc2nn3c(c2CN1C(=O)c1ccc(Cl)c(Cl)c1)C(=O)N([C@@H](C)c1ccc(-n2cncn2)nc1)C[C@H]3C]

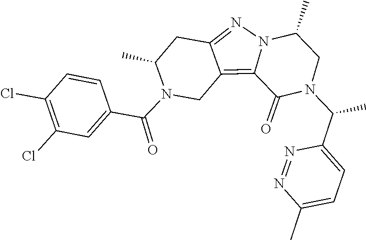 [SMILES: Cc1ccc([C@@H](C)N2C[C@@H](C)n3nc4c(c3C2=O)CN(C(=O)c2ccc(Cl)c(Cl)c2)[C@H](C)C4)nn1]

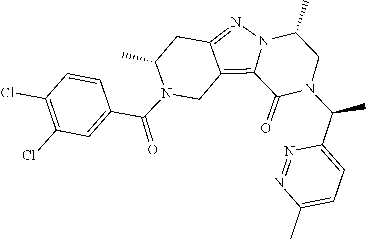 [SMILES: Cc1ccc([C@H](C)N2C[C@@H](C)n3nc4c(c3C2=O)CN(C(=O)c2ccc(Cl)c(Cl)c2)[C@H](C)C4)nn1]

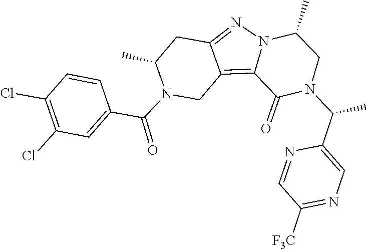 [SMILES: C[C@@H]1Cc2nn3c(c2CN1C(=O)c1ccc(Cl)c(Cl)c1)C(=O)N([C@H](C)c1cnc(C(F)(F)F)cn1)C[C@H]3C]

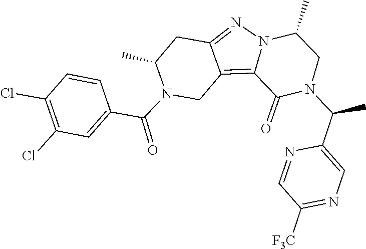 [SMILES: C[C@@H]1Cc2nn3c(c2CN1C(=O)c1ccc(Cl)c(Cl)c1)C(=O)N([C@@H](C)c1cnc(C(F)(F)F)cn1)C[C@H]3C]

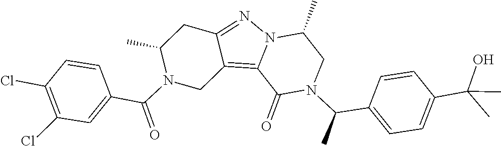 [SMILES: C[C@@H]1Cc2nn3c(c2CN1C(=O)c1ccc(Cl)c(Cl)c1)C(=O)N([C@H](C)c1ccc(C(C)(C)O)cc1)C[C@H]3C]

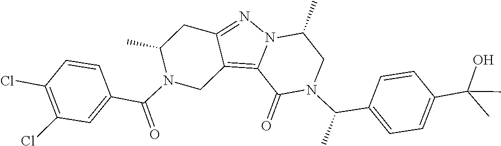 [SMILES: C[C@@H]1Cc2nn3c(c2CN1C(=O)c1ccc(Cl)c(Cl)c1)C(=O)N([C@@H](C)c1ccc(C(C)(C)O)cc1)C[C@H]3C]

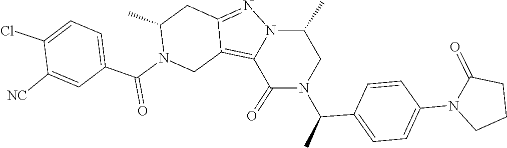 [SMILES: C[C@@H]1Cc2nn3c(c2CN1C(=O)c1ccc(Cl)c(C#N)c1)C(=O)N([C@H](C)c1ccc(N2CCCC2=O)cc1)C[C@H]3C]

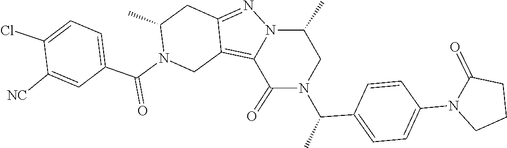 [SMILES: C[C@@H]1Cc2nn3c(c2CN1C(=O)c1ccc(Cl)c(C#N)c1)C(=O)N([C@@H](C)c1ccc(N2CCCC2=O)cc1)C[C@H]3C]

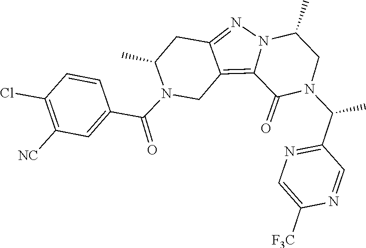 [SMILES: C[C@@H]1Cc2nn3c(c2CN1C(=O)c1ccc(Cl)c(C#N)c1)C(=O)N([C@H](C)c1cnc(C(F)(F)F)cn1)C[C@H]3C]

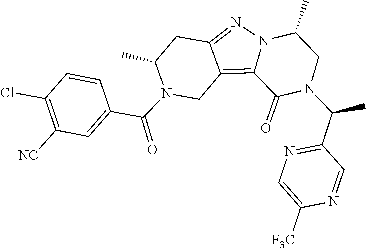 [SMILES: C[C@@H]1Cc2nn3c(c2CN1C(=O)c1ccc(Cl)c(C#N)c1)C(=O)N([C@@H](C)c1cnc(C(F)(F)F)cn1)C[C@H]3C]

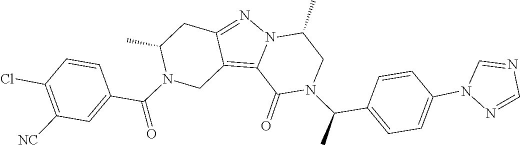 [SMILES: C[C@@H]1Cc2nn3c(c2CN1C(=O)c1ccc(Cl)c(C#N)c1)C(=O)N([C@H](C)c1ccc(-n2cncn2)cc1)C[C@H]3C]

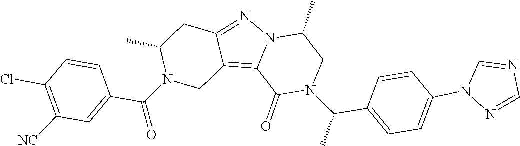 [SMILES: C[C@@H]1Cc2nn3c(c2CN1C(=O)c1ccc(Cl)c(C#N)c1)C(=O)N([C@@H](C)c1ccc(-n2cncn2)cc1)C[C@H]3C]

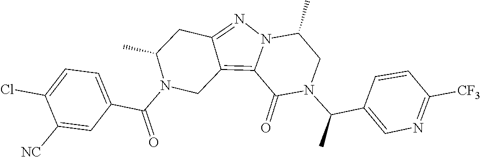 [SMILES: C[C@@H]1Cc2nn3c(c2CN1C(=O)c1ccc(Cl)c(C#N)c1)C(=O)N([C@H](C)c1ccc(C(F)(F)F)nc1)C[C@H]3C]

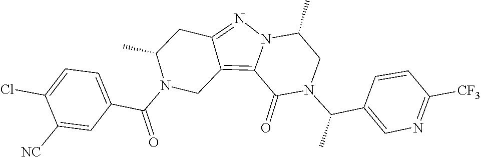 [SMILES: C[C@@H]1Cc2nn3c(c2CN1C(=O)c1ccc(Cl)c(C#N)c1)C(=O)N([C@@H](C)c1ccc(C(F)(F)F)nc1)C[C@H]3C]

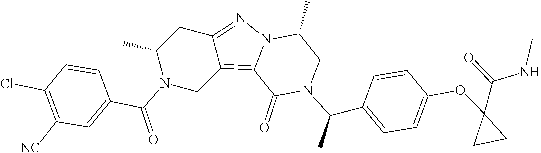 [SMILES: CNC(=O)C1(Oc2ccc([C@@H](C)N3C[C@@H](C)n4nc5c(c4C3=O)CN(C(=O)c3ccc(Cl)c(C#N)c3)[C@H](C)C5)cc2)CC1]

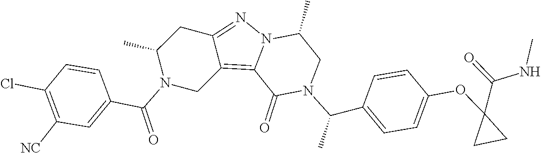 [SMILES: CNC(=O)C1(Oc2ccc([C@H](C)N3C[C@@H](C)n4nc5c(c4C3=O)CN(C(=O)c3ccc(Cl)c(C#N)c3)[C@H](C)C5)cc2)CC1]

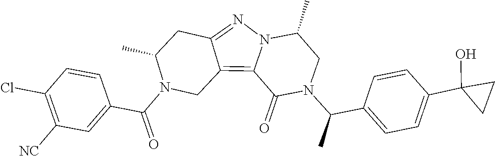 [SMILES: C[C@@H]1Cc2nn3c(c2CN1C(=O)c1ccc(Cl)c(C#N)c1)C(=O)N([C@H](C)c1ccc(C2(O)CC2)cc1)C[C@H]3C]

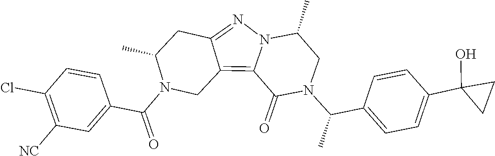 [SMILES: C[C@@H]1Cc2nn3c(c2CN1C(=O)c1ccc(Cl)c(C#N)c1)C(=O)N([C@@H](C)c1ccc(C2(O)CC2)cc1)C[C@H]3C]

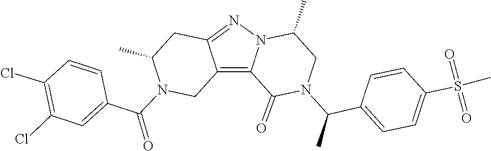 [SMILES: C[C@@H]1Cc2nn3c(c2CN1C(=O)c1ccc(Cl)c(Cl)c1)C(=O)N([C@H](C)c1ccc(S(C)(=O)=O)cc1)C[C@H]3C]

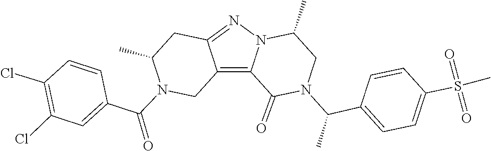 [SMILES: C[C@@H]1Cc2nn3c(c2CN1C(=O)c1ccc(Cl)c(Cl)c1)C(=O)N([C@@H](C)c1ccc(S(C)(=O)=O)cc1)C[C@H]3C]

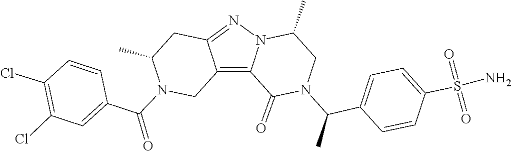 [SMILES: C[C@@H]1Cc2nn3c(c2CN1C(=O)c1ccc(Cl)c(Cl)c1)C(=O)N([C@H](C)c1ccc(S(N)(=O)=O)cc1)C[C@H]3C]

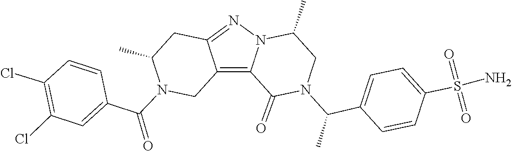 [SMILES: C[C@@H]1Cc2nn3c(c2CN1C(=O)c1ccc(Cl)c(Cl)c1)C(=O)N([C@@H](C)c1ccc(S(N)(=O)=O)cc1)C[C@H]3C]

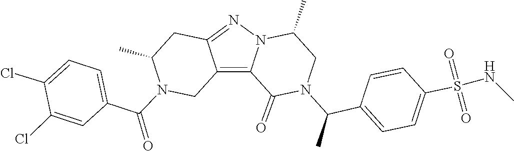 [SMILES: CNS(=O)(=O)c1ccc([C@@H](C)N2C[C@@H](C)n3nc4c(c3C2=O)CN(C(=O)c2ccc(Cl)c(Cl)c2)[C@H](C)C4)cc1]

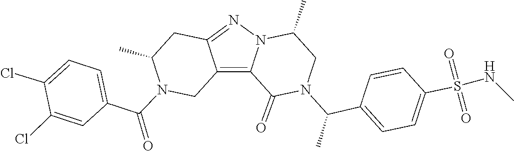 [SMILES: CNS(=O)(=O)c1ccc([C@H](C)N2C[C@@H](C)n3nc4c(c3C2=O)CN(C(=O)c2ccc(Cl)c(Cl)c2)[C@H](C)C4)cc1]

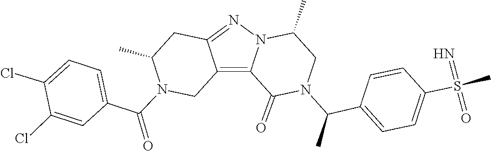 [SMILES: C[C@@H]1Cc2nn3c(c2CN1C(=O)c1ccc(Cl)c(Cl)c1)C(=O)N([C@H](C)c1ccc([S@](C)(=N)=O)cc1)C[C@H]3C]

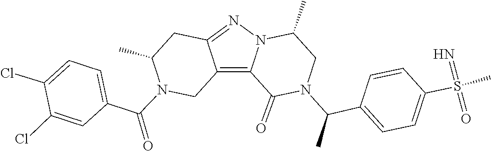 [SMILES: C[C@@H]1Cc2nn3c(c2CN1C(=O)c1ccc(Cl)c(Cl)c1)C(=O)N([C@H](C)c1ccc([S@@](C)(=N)=O)cc1)C[C@H]3C]

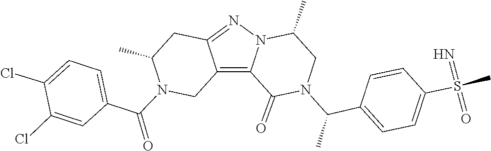 [SMILES: C[C@@H]1Cc2nn3c(c2CN1C(=O)c1ccc(Cl)c(Cl)c1)C(=O)N([C@@H](C)c1ccc([S@](C)(=N)=O)cc1)C[C@H]3C]

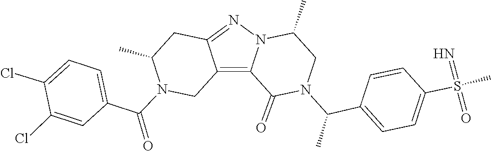 [SMILES: C[C@@H]1Cc2nn3c(c2CN1C(=O)c1ccc(Cl)c(Cl)c1)C(=O)N([C@@H](C)c1ccc([S@@](C)(=N)=O)cc1)C[C@H]3C]

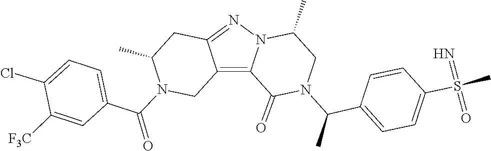 [SMILES: C[C@@H]1Cc2nn3c(c2CN1C(=O)c1ccc(Cl)c(C(F)(F)F)c1)C(=O)N([C@H](C)c1ccc([S@](C)(=N)=O)cc1)C[C@H]3C]

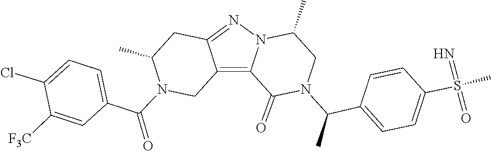 [SMILES: C[C@@H]1Cc2nn3c(c2CN1C(=O)c1ccc(Cl)c(C(F)(F)F)c1)C(=O)N([C@H](C)c1ccc([S@@](C)(=N)=O)cc1)C[C@H]3C]

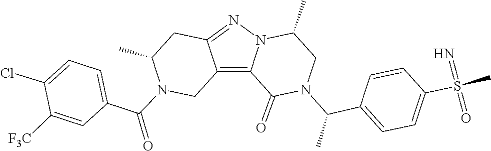 [SMILES: C[C@@H]1Cc2nn3c(c2CN1C(=O)c1ccc(Cl)c(C(F)(F)F)c1)C(=O)N([C@@H](C)c1ccc([S@](C)(=N)=O)cc1)C[C@H]3C]

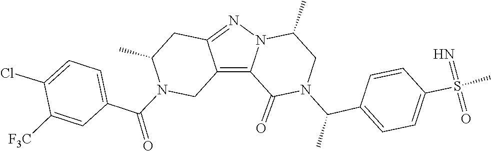 [SMILES: C[C@@H]1Cc2nn3c(c2CN1C(=O)c1ccc(Cl)c(C(F)(F)F)c1)C(=O)N([C@@H](C)c1ccc([S@@](C)(=N)=O)cc1)C[C@H]3C]

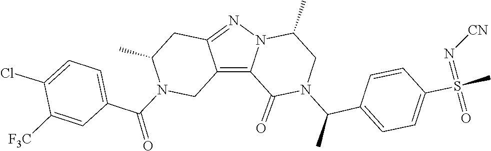 [SMILES: C[C@@H]1Cc2nn3c(c2CN1C(=O)c1ccc(Cl)c(C(F)(F)F)c1)C(=O)N([C@H](C)c1ccc([S@@](C)(=O)=NC#N)cc1)C[C@H]3C]

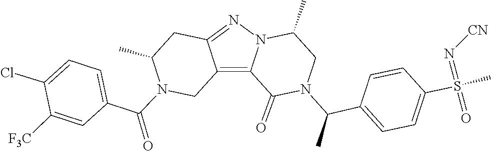 [SMILES: C[C@@H]1Cc2nn3c(c2CN1C(=O)c1ccc(Cl)c(C(F)(F)F)c1)C(=O)N([C@H](C)c1ccc([S@](C)(=O)=NC#N)cc1)C[C@H]3C]

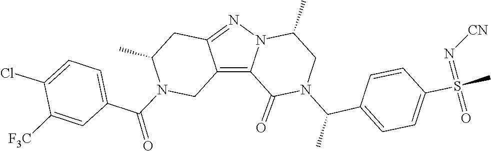 [SMILES: C[C@@H]1Cc2nn3c(c2CN1C(=O)c1ccc(Cl)c(C(F)(F)F)c1)C(=O)N([C@@H](C)c1ccc([S@@](C)(=O)=NC#N)cc1)C[C@H]3C]

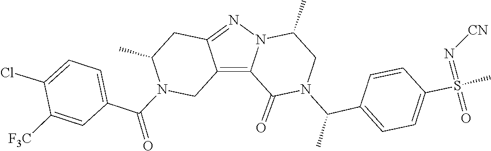 [SMILES: C[C@@H]1Cc2nn3c(c2CN1C(=O)c1ccc(Cl)c(C(F)(F)F)c1)C(=O)N([C@@H](C)c1ccc([S@](C)(=O)=NC#N)cc1)C[C@H]3C]

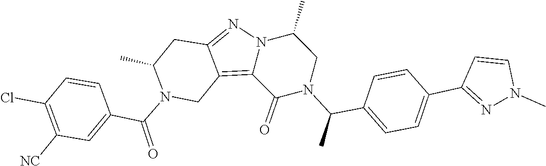 [SMILES: C[C@@H]1Cc2nn3c(c2CN1C(=O)c1ccc(Cl)c(C#N)c1)C(=O)N([C@H](C)c1ccc(-c2ccn(C)n2)cc1)C[C@H]3C]

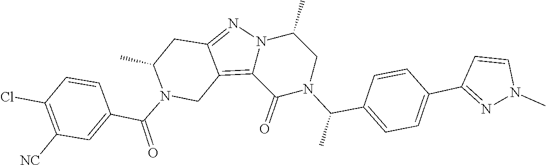 [SMILES: C[C@@H]1Cc2nn3c(c2CN1C(=O)c1ccc(Cl)c(C#N)c1)C(=O)N([C@@H](C)c1ccc(-c2ccn(C)n2)cc1)C[C@H]3C]

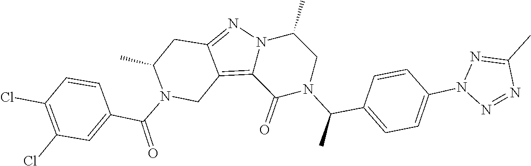 [SMILES: Cc1nnn(-c2ccc([C@@H](C)N3C[C@@H](C)n4nc5c(c4C3=O)CN(C(=O)c3ccc(Cl)c(Cl)c3)[C@H](C)C5)cc2)n1]

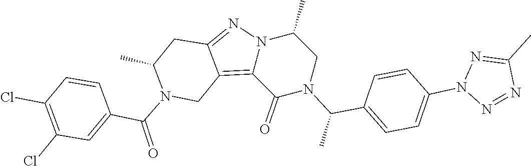 [SMILES: Cc1nnn(-c2ccc([C@H](C)N3C[C@@H](C)n4nc5c(c4C3=O)CN(C(=O)c3ccc(Cl)c(Cl)c3)[C@H](C)C5)cc2)n1]

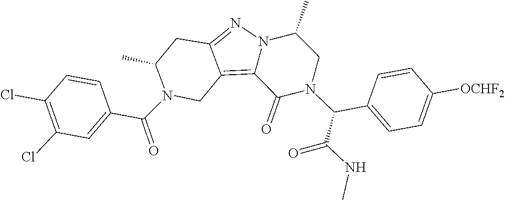 [SMILES: CNC(=O)[C@@H](c1ccc(OC(F)F)cc1)N1C[C@@H](C)n2nc3c(c2C1=O)CN(C(=O)c1ccc(Cl)c(Cl)c1)[C@H](C)C3]

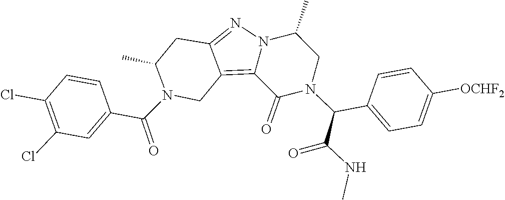 [SMILES: CNC(=O)[C@H](c1ccc(OC(F)F)cc1)N1C[C@@H](C)n2nc3c(c2C1=O)CN(C(=O)c1ccc(Cl)c(Cl)c1)[C@H](C)C3]

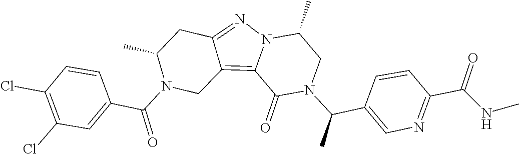 [SMILES: CNC(=O)c1ccc([C@@H](C)N2C[C@@H](C)n3nc4c(c3C2=O)CN(C(=O)c2ccc(Cl)c(Cl)c2)[C@H](C)C4)cn1]

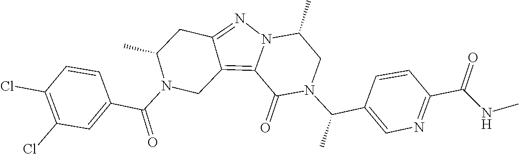 [SMILES: CNC(=O)c1ccc([C@H](C)N2C[C@@H](C)n3nc4c(c3C2=O)CN(C(=O)c2ccc(Cl)c(Cl)c2)[C@H](C)C4)cn1]

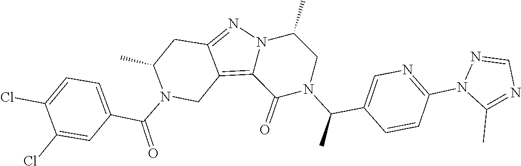 [SMILES: Cc1ncnn1-c1ccc([C@@H](C)N2C[C@@H](C)n3nc4c(c3C2=O)CN(C(=O)c2ccc(Cl)c(Cl)c2)[C@H](C)C4)cn1]

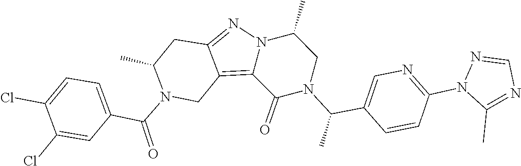 [SMILES: Cc1ncnn1-c1ccc([C@H](C)N2C[C@@H](C)n3nc4c(c3C2=O)CN(C(=O)c2ccc(Cl)c(Cl)c2)[C@H](C)C4)cn1]

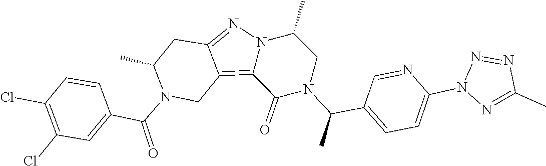 [SMILES: Cc1nnn(-c2ccc([C@@H](C)N3C[C@@H](C)n4nc5c(c4C3=O)CN(C(=O)c3ccc(Cl)c(Cl)c3)[C@H](C)C5)cn2)n1]

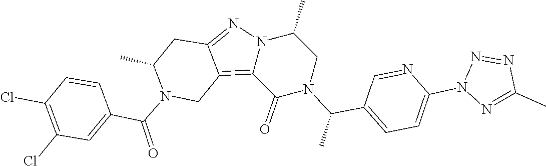 [SMILES: Cc1nnn(-c2ccc([C@H](C)N3C[C@@H](C)n4nc5c(c4C3=O)CN(C(=O)c3ccc(Cl)c(Cl)c3)[C@H](C)C5)cn2)n1]

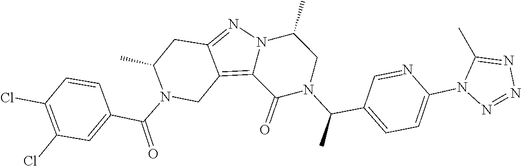 [SMILES: Cc1nnnn1-c1ccc([C@@H](C)N2C[C@@H](C)n3nc4c(c3C2=O)CN(C(=O)c2ccc(Cl)c(Cl)c2)[C@H](C)C4)cn1]

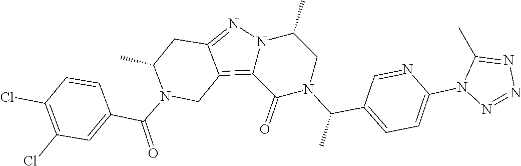 [SMILES: Cc1nnnn1-c1ccc([C@H](C)N2C[C@@H](C)n3nc4c(c3C2=O)CN(C(=O)c2ccc(Cl)c(Cl)c2)[C@H](C)C4)cn1]

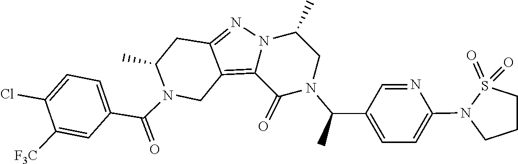 [SMILES: C[C@@H]1Cc2nn3c(c2CN1C(=O)c1ccc(Cl)c(C(F)(F)F)c1)C(=O)N([C@H](C)c1ccc(N2CCCS2(=O)=O)nc1)C[C@H]3C]

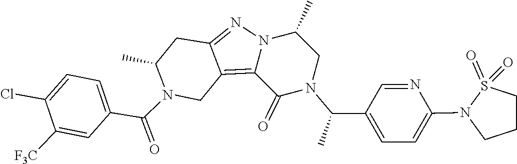 [SMILES: C[C@@H]1Cc2nn3c(c2CN1C(=O)c1ccc(Cl)c(C(F)(F)F)c1)C(=O)N([C@@H](C)c1ccc(N2CCCS2(=O)=O)nc1)C[C@H]3C]

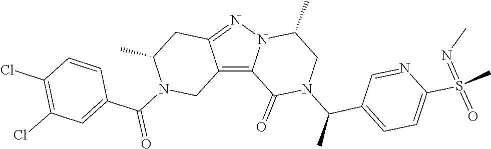 [SMILES: CN=[S@](C)(=O)c1ccc([C@@H](C)N2C[C@@H](C)n3nc4c(c3C2=O)CN(C(=O)c2ccc(Cl)c(Cl)c2)[C@H](C)C4)cn1]

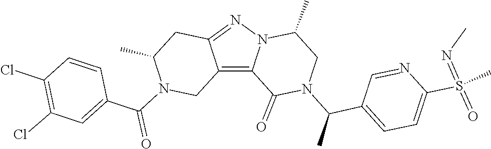 [SMILES: CN=[S@@](C)(=O)c1ccc([C@@H](C)N2C[C@@H](C)n3nc4c(c3C2=O)CN(C(=O)c2ccc(Cl)c(Cl)c2)[C@H](C)C4)cn1]

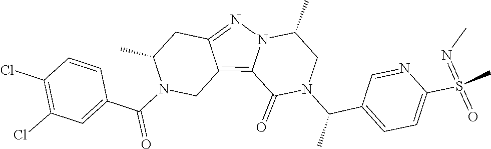 [SMILES: CN=[S@](C)(=O)c1ccc([C@H](C)N2C[C@@H](C)n3nc4c(c3C2=O)CN(C(=O)c2ccc(Cl)c(Cl)c2)[C@H](C)C4)cn1]

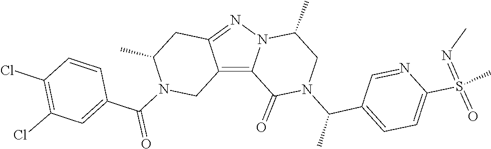 [SMILES: CN=[S@@](C)(=O)c1ccc([C@H](C)N2C[C@@H](C)n3nc4c(c3C2=O)CN(C(=O)c2ccc(Cl)c(Cl)c2)[C@H](C)C4)cn1]

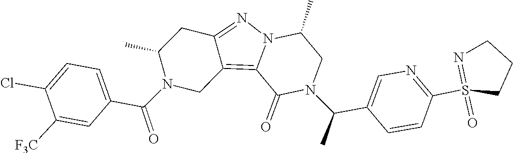 [SMILES: C[C@@H]1Cc2nn3c(c2CN1C(=O)c1ccc(Cl)c(C(F)(F)F)c1)C(=O)N([C@H](C)c1ccc([S@@]2(=O)=NCCC2)nc1)C[C@H]3C]

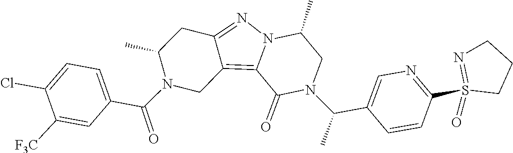 [SMILES: C[C@@H]1Cc2nn3c(c2CN1C(=O)c1ccc(Cl)c(C(F)(F)F)c1)C(=O)N([C@@H](C)c1ccc([S@@]2(=O)=NCCC2)nc1)C[C@H]3C]

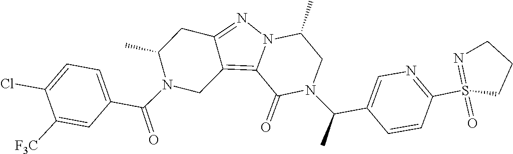 [SMILES: C[C@@H]1Cc2nn3c(c2CN1C(=O)c1ccc(Cl)c(C(F)(F)F)c1)C(=O)N([C@H](C)c1ccc([S@]2(=O)=NCCC2)nc1)C[C@H]3C]

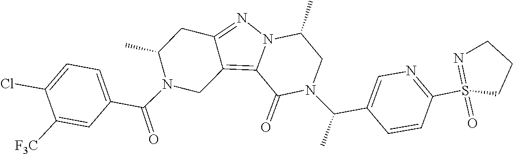 [SMILES: C[C@@H]1Cc2nn3c(c2CN1C(=O)c1ccc(Cl)c(C(F)(F)F)c1)C(=O)N([C@@H](C)c1ccc([S@]2(=O)=NCCC2)nc1)C[C@H]3C]

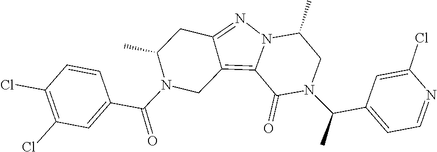 [SMILES: C[C@@H]1Cc2nn3c(c2CN1C(=O)c1ccc(Cl)c(Cl)c1)C(=O)N([C@H](C)c1ccnc(Cl)c1)C[C@H]3C]

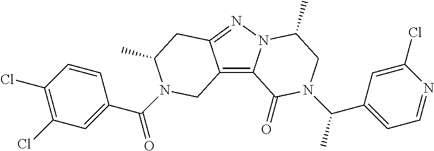 [SMILES: C[C@@H]1Cc2nn3c(c2CN1C(=O)c1ccc(Cl)c(Cl)c1)C(=O)N([C@@H](C)c1ccnc(Cl)c1)C[C@H]3C]

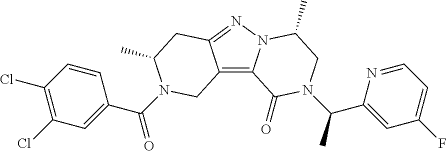 [SMILES: C[C@@H]1Cc2nn3c(c2CN1C(=O)c1ccc(Cl)c(Cl)c1)C(=O)N([C@H](C)c1cc(F)ccn1)C[C@H]3C]

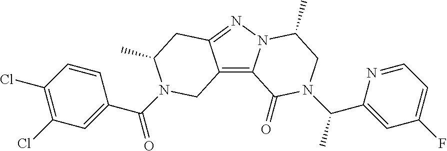 [SMILES: C[C@@H]1Cc2nn3c(c2CN1C(=O)c1ccc(Cl)c(Cl)c1)C(=O)N([C@@H](C)c1cc(F)ccn1)C[C@H]3C]

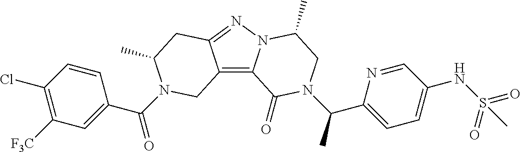 [SMILES: C[C@@H]1Cc2nn3c(c2CN1C(=O)c1ccc(Cl)c(C(F)(F)F)c1)C(=O)N([C@H](C)c1ccc(NS(C)(=O)=O)cn1)C[C@H]3C]

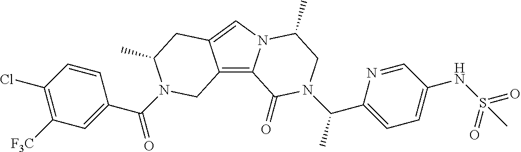 [SMILES: C[C@@H]1Cc2cn3c(c2CN1C(=O)c1ccc(Cl)c(C(F)(F)F)c1)C(=O)N([C@@H](C)c1ccc(NS(C)(=O)=O)cn1)C[C@H]3C]

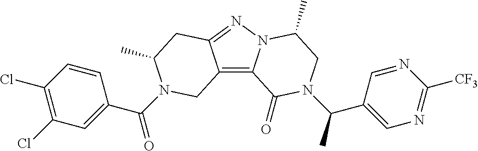 [SMILES: C[C@@H]1Cc2nn3c(c2CN1C(=O)c1ccc(Cl)c(Cl)c1)C(=O)N([C@H](C)c1cnc(C(F)(F)F)nc1)C[C@H]3C]

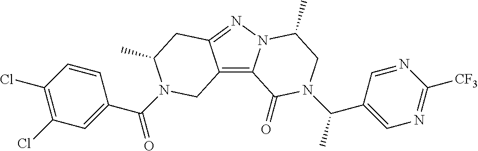 [SMILES: C[C@@H]1Cc2nn3c(c2CN1C(=O)c1ccc(Cl)c(Cl)c1)C(=O)N([C@@H](C)c1cnc(C(F)(F)F)nc1)C[C@H]3C]

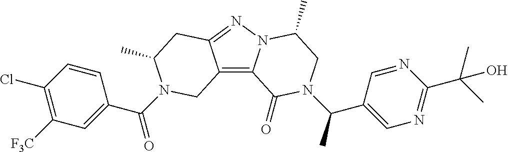 [SMILES: C[C@@H]1Cc2nn3c(c2CN1C(=O)c1ccc(Cl)c(C(F)(F)F)c1)C(=O)N([C@H](C)c1cnc(C(C)(C)O)nc1)C[C@H]3C]

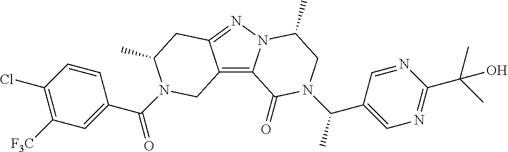 [SMILES: C[C@@H]1Cc2nn3c(c2CN1C(=O)c1ccc(Cl)c(C(F)(F)F)c1)C(=O)N([C@@H](C)c1cnc(C(C)(C)O)nc1)C[C@H]3C]

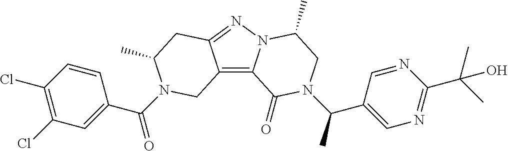 [SMILES: C[C@@H]1Cc2nn3c(c2CN1C(=O)c1ccc(Cl)c(Cl)c1)C(=O)N([C@H](C)c1cnc(C(C)(C)O)nc1)C[C@H]3C]

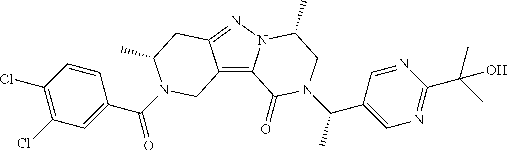 [SMILES: C[C@@H]1Cc2nn3c(c2CN1C(=O)c1ccc(Cl)c(Cl)c1)C(=O)N([C@@H](C)c1cnc(C(C)(C)O)nc1)C[C@H]3C]

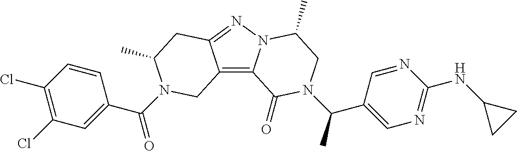 [SMILES: C[C@@H]1Cc2nn3c(c2CN1C(=O)c1ccc(Cl)c(Cl)c1)C(=O)N([C@H](C)c1cnc(NC2CC2)nc1)C[C@H]3C]

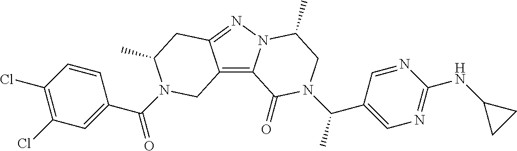 [SMILES: C[C@@H]1Cc2nn3c(c2CN1C(=O)c1ccc(Cl)c(Cl)c1)C(=O)N([C@@H](C)c1cnc(NC2CC2)nc1)C[C@H]3C]

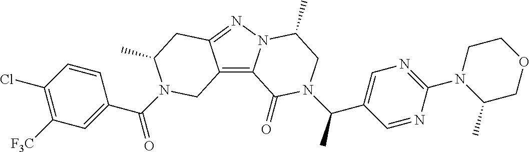 [SMILES: C[C@@H]1Cc2nn3c(c2CN1C(=O)c1ccc(Cl)c(C(F)(F)F)c1)C(=O)N([C@H](C)c1cnc(N2CCOC[C@@H]2C)nc1)C[C@H]3C]

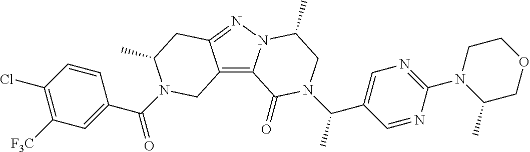 [SMILES: C[C@@H]1Cc2nn3c(c2CN1C(=O)c1ccc(Cl)c(C(F)(F)F)c1)C(=O)N([C@@H](C)c1cnc(N2CCOC[C@@H]2C)nc1)C[C@H]3C]

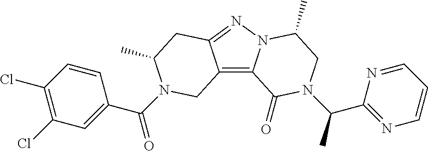 [SMILES: C[C@@H]1Cc2nn3c(c2CN1C(=O)c1ccc(Cl)c(Cl)c1)C(=O)N([C@H](C)c1ncccn1)C[C@H]3C]

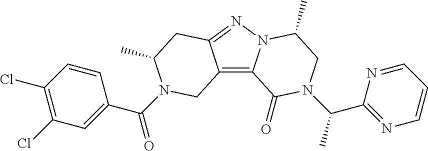 [SMILES: C[C@@H]1Cc2nn3c(c2CN1C(=O)c1ccc(Cl)c(Cl)c1)C(=O)N([C@@H](C)c1ncccn1)C[C@H]3C]

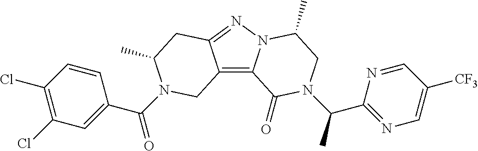 [SMILES: C[C@@H]1Cc2nn3c(c2CN1C(=O)c1ccc(Cl)c(Cl)c1)C(=O)N([C@H](C)c1ncc(C(F)(F)F)cn1)C[C@H]3C]

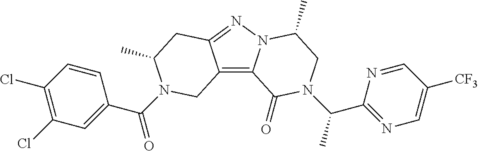 [SMILES: C[C@@H]1Cc2nn3c(c2CN1C(=O)c1ccc(Cl)c(Cl)c1)C(=O)N([C@@H](C)c1ncc(C(F)(F)F)cn1)C[C@H]3C]

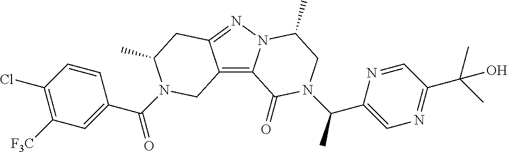 [SMILES: C[C@@H]1Cc2nn3c(c2CN1C(=O)c1ccc(Cl)c(C(F)(F)F)c1)C(=O)N([C@H](C)c1cnc(C(C)(C)O)cn1)C[C@H]3C]

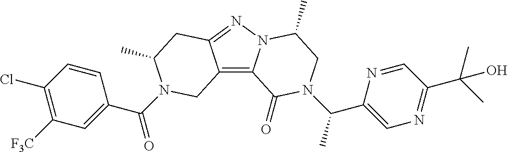 [SMILES: C[C@@H]1Cc2nn3c(c2CN1C(=O)c1ccc(Cl)c(C(F)(F)F)c1)C(=O)N([C@@H](C)c1cnc(C(C)(C)O)cn1)C[C@H]3C]

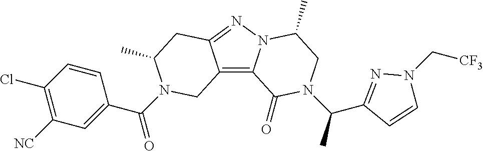 [SMILES: C[C@@H]1Cc2nn3c(c2CN1C(=O)c1ccc(Cl)c(C#N)c1)C(=O)N([C@H](C)c1ccn(CC(F)(F)F)n1)C[C@H]3C]

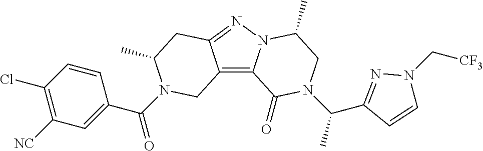 [SMILES: C[C@@H]1Cc2nn3c(c2CN1C(=O)c1ccc(Cl)c(C#N)c1)C(=O)N([C@@H](C)c1ccn(CC(F)(F)F)n1)C[C@H]3C]

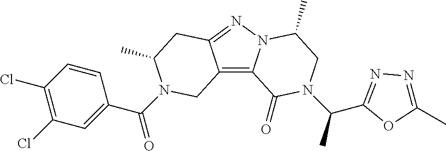 [SMILES: Cc1nnc([C@@H](C)N2C[C@@H](C)n3nc4c(c3C2=O)CN(C(=O)c2ccc(Cl)c(Cl)c2)[C@H](C)C4)o1]

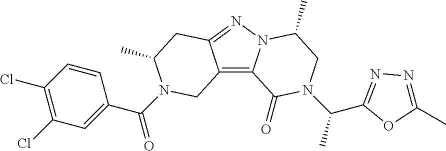 [SMILES: Cc1nnc([C@H](C)N2C[C@@H](C)n3nc4c(c3C2=O)CN(C(=O)c2ccc(Cl)c(Cl)c2)[C@H](C)C4)o1]

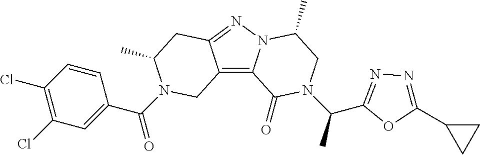 [SMILES: C[C@@H]1Cc2nn3c(c2CN1C(=O)c1ccc(Cl)c(Cl)c1)C(=O)N([C@H](C)c1nnc(C2CC2)o1)C[C@H]3C]

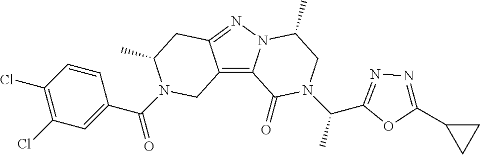 [SMILES: C[C@@H]1Cc2nn3c(c2CN1C(=O)c1ccc(Cl)c(Cl)c1)C(=O)N([C@@H](C)c1nnc(C2CC2)o1)C[C@H]3C]

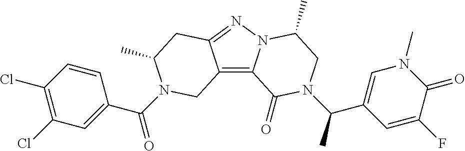 [SMILES: C[C@@H]1Cc2nn3c(c2CN1C(=O)c1ccc(Cl)c(Cl)c1)C(=O)N([C@H](C)c1cc(F)c(=O)n(C)c1)C[C@H]3C]

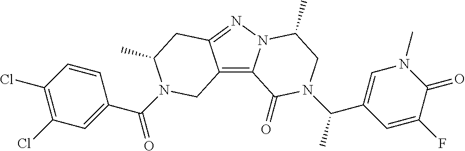 [SMILES: C[C@@H]1Cc2nn3c(c2CN1C(=O)c1ccc(Cl)c(Cl)c1)C(=O)N([C@@H](C)c1cc(F)c(=O)n(C)c1)C[C@H]3C]

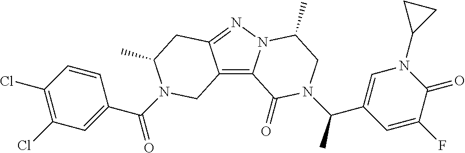 [SMILES: C[C@@H]1Cc2nn3c(c2CN1C(=O)c1ccc(Cl)c(Cl)c1)C(=O)N([C@H](C)c1cc(F)c(=O)n(C2CC2)c1)C[C@H]3C]

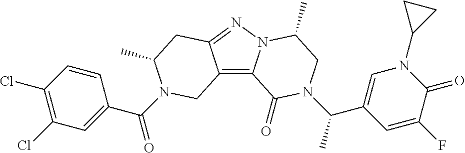 [SMILES: C[C@@H]1Cc2nn3c(c2CN1C(=O)c1ccc(Cl)c(Cl)c1)C(=O)N([C@@H](C)c1cc(F)c(=O)n(C2CC2)c1)C[C@H]3C]

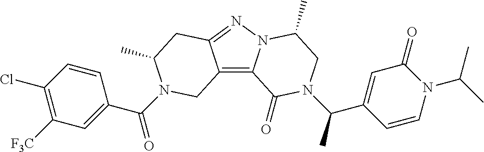 [SMILES: CC(C)n1ccc([C@@H](C)N2C[C@@H](C)n3nc4c(c3C2=O)CN(C(=O)c2ccc(Cl)c(C(F)(F)F)c2)[C@H](C)C4)cc1=O]

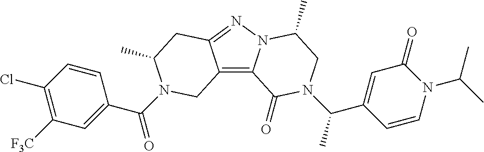 [SMILES: CC(C)n1ccc([C@H](C)N2C[C@@H](C)n3nc4c(c3C2=O)CN(C(=O)c2ccc(Cl)c(C(F)(F)F)c2)[C@H](C)C4)cc1=O]

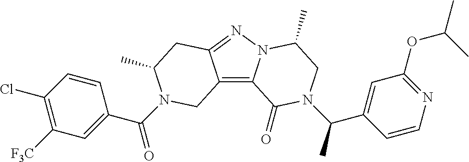 [SMILES: CC(C)Oc1cc([C@@H](C)N2C[C@@H](C)n3nc4c(c3C2=O)CN(C(=O)c2ccc(Cl)c(C(F)(F)F)c2)[C@H](C)C4)ccn1]

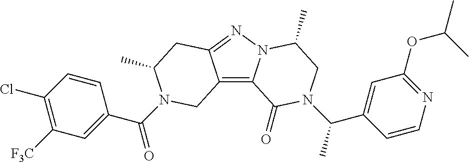 [SMILES: CC(C)Oc1cc([C@H](C)N2C[C@@H](C)n3nc4c(c3C2=O)CN(C(=O)c2ccc(Cl)c(C(F)(F)F)c2)[C@H](C)C4)ccn1]

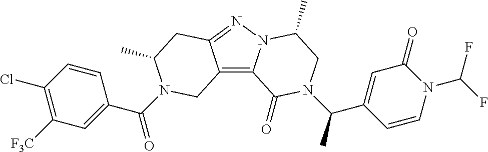 [SMILES: C[C@@H]1Cc2nn3c(c2CN1C(=O)c1ccc(Cl)c(C(F)(F)F)c1)C(=O)N([C@H](C)c1ccn(C(F)F)c(=O)c1)C[C@H]3C]

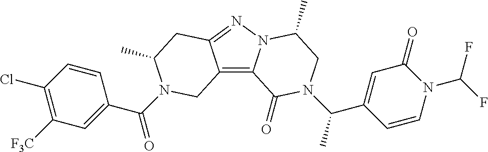 [SMILES: C[C@@H]1Cc2nn3c(c2CN1C(=O)c1ccc(Cl)c(C(F)(F)F)c1)C(=O)N([C@@H](C)c1ccn(C(F)F)c(=O)c1)C[C@H]3C]

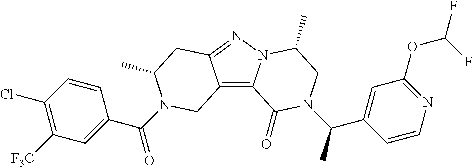 [SMILES: C[C@@H]1Cc2nn3c(c2CN1C(=O)c1ccc(Cl)c(C(F)(F)F)c1)C(=O)N([C@H](C)c1ccnc(OC(F)F)c1)C[C@H]3C]

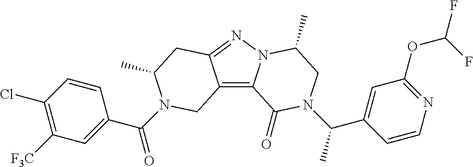 [SMILES: C[C@@H]1Cc2nn3c(c2CN1C(=O)c1ccc(Cl)c(C(F)(F)F)c1)C(=O)N([C@@H](C)c1ccnc(OC(F)F)c1)C[C@H]3C]

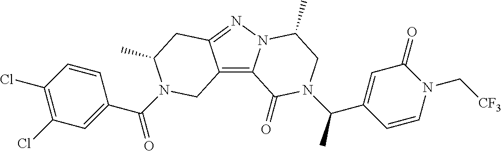 [SMILES: C[C@@H]1Cc2nn3c(c2CN1C(=O)c1ccc(Cl)c(Cl)c1)C(=O)N([C@H](C)c1ccn(CC(F)(F)F)c(=O)c1)C[C@H]3C]

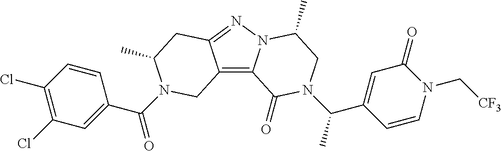 [SMILES: C[C@@H]1Cc2nn3c(c2CN1C(=O)c1ccc(Cl)c(Cl)c1)C(=O)N([C@@H](C)c1ccn(CC(F)(F)F)c(=O)c1)C[C@H]3C]

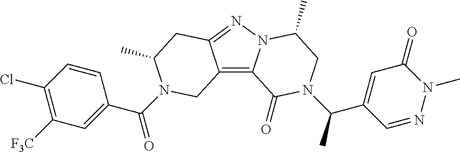 [SMILES: C[C@@H]1Cc2nn3c(c2CN1C(=O)c1ccc(Cl)c(C(F)(F)F)c1)C(=O)N([C@H](C)c1cnn(C)c(=O)c1)C[C@H]3C]

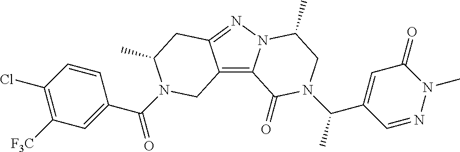 [SMILES: C[C@@H]1Cc2nn3c(c2CN1C(=O)c1ccc(Cl)c(C(F)(F)F)c1)C(=O)N([C@@H](C)c1cnn(C)c(=O)c1)C[C@H]3C]

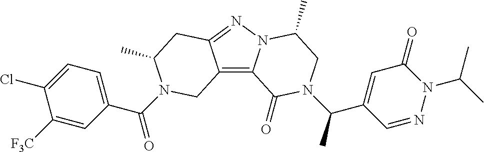 [SMILES: CC(C)n1ncc([C@@H](C)N2C[C@@H](C)n3nc4c(c3C2=O)CN(C(=O)c2ccc(Cl)c(C(F)(F)F)c2)[C@H](C)C4)cc1=O]

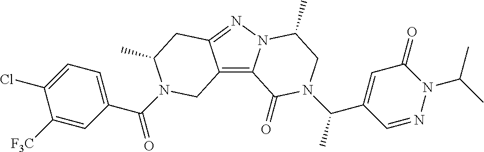 [SMILES: CC(C)n1ncc([C@H](C)N2C[C@@H](C)n3nc4c(c3C2=O)CN(C(=O)c2ccc(Cl)c(C(F)(F)F)c2)[C@H](C)C4)cc1=O]

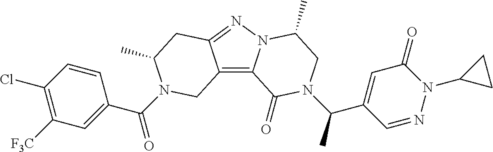 [SMILES: C[C@@H]1Cc2nn3c(c2CN1C(=O)c1ccc(Cl)c(C(F)(F)F)c1)C(=O)N([C@H](C)c1cnn(C2CC2)c(=O)c1)C[C@H]3C]

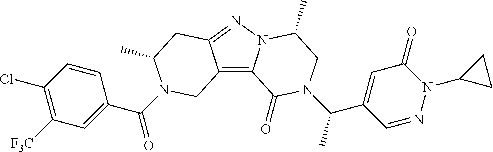 [SMILES: C[C@@H]1Cc2nn3c(c2CN1C(=O)c1ccc(Cl)c(C(F)(F)F)c1)C(=O)N([C@@H](C)c1cnn(C2CC2)c(=O)c1)C[C@H]3C]

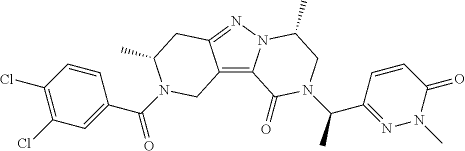 [SMILES: C[C@@H]1Cc2nn3c(c2CN1C(=O)c1ccc(Cl)c(Cl)c1)C(=O)N([C@H](C)c1ccc(=O)n(C)n1)C[C@H]3C]

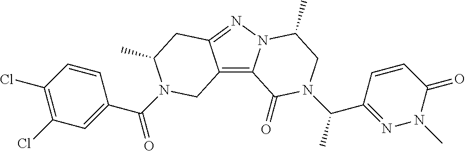 [SMILES: C[C@@H]1Cc2nn3c(c2CN1C(=O)c1ccc(Cl)c(Cl)c1)C(=O)N([C@@H](C)c1ccc(=O)n(C)n1)C[C@H]3C]

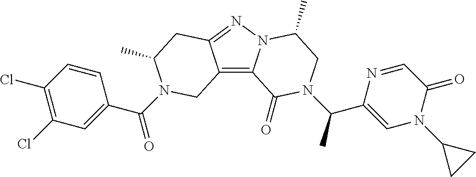 [SMILES: C[C@@H]1Cc2nn3c(c2CN1C(=O)c1ccc(Cl)c(Cl)c1)C(=O)N([C@H](C)c1cn(C2CC2)c(=O)cn1)C[C@H]3C]

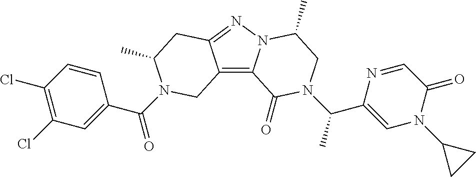 [SMILES: C[C@@H]1Cc2nn3c(c2CN1C(=O)c1ccc(Cl)c(Cl)c1)C(=O)N([C@@H](C)c1cn(C2CC2)c(=O)cn1)C[C@H]3C]

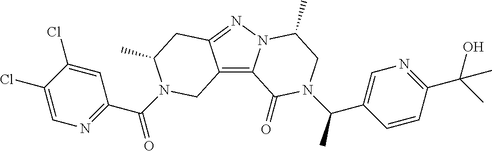 [SMILES: C[C@@H]1Cc2nn3c(c2CN1C(=O)c1cc(Cl)c(Cl)cn1)C(=O)N([C@H](C)c1ccc(C(C)(C)O)nc1)C[C@H]3C]

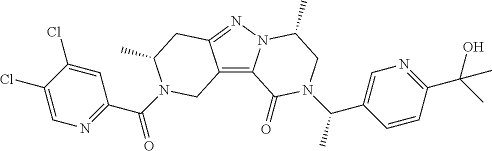 [SMILES: C[C@@H]1Cc2nn3c(c2CN1C(=O)c1cc(Cl)c(Cl)cn1)C(=O)N([C@@H](C)c1ccc(C(C)(C)O)nc1)C[C@H]3C]

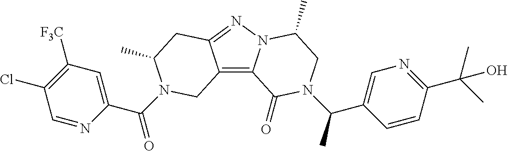 [SMILES: C[C@@H]1Cc2nn3c(c2CN1C(=O)c1cc(C(F)(F)F)c(Cl)cn1)C(=O)N([C@H](C)c1ccc(C(C)(C)O)nc1)C[C@H]3C]

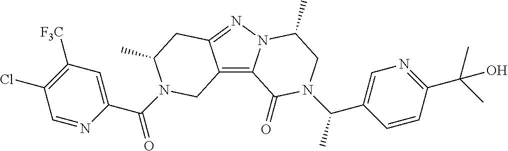 [SMILES: C[C@@H]1Cc2nn3c(c2CN1C(=O)c1cc(C(F)(F)F)c(Cl)cn1)C(=O)N([C@@H](C)c1ccc(C(C)(C)O)nc1)C[C@H]3C]

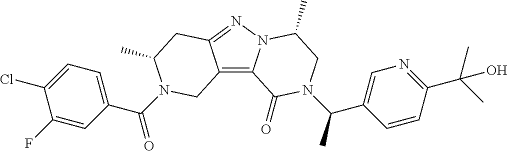 [SMILES: C[C@@H]1Cc2nn3c(c2CN1C(=O)c1ccc(Cl)c(F)c1)C(=O)N([C@H](C)c1ccc(C(C)(C)O)nc1)C[C@H]3C]

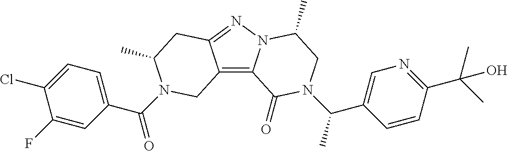 [SMILES: C[C@@H]1Cc2nn3c(c2CN1C(=O)c1ccc(Cl)c(F)c1)C(=O)N([C@@H](C)c1ccc(C(C)(C)O)nc1)C[C@H]3C]

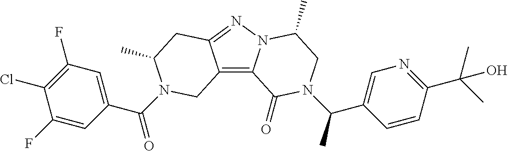 [SMILES: C[C@@H]1Cc2nn3c(c2CN1C(=O)c1cc(F)c(Cl)c(F)c1)C(=O)N([C@H](C)c1ccc(C(C)(C)O)nc1)C[C@H]3C]

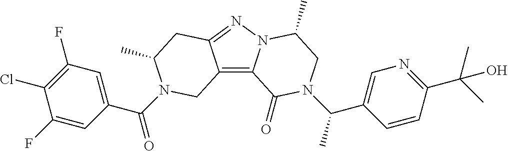 [SMILES: C[C@@H]1Cc2nn3c(c2CN1C(=O)c1cc(F)c(Cl)c(F)c1)C(=O)N([C@@H](C)c1ccc(C(C)(C)O)nc1)C[C@H]3C]

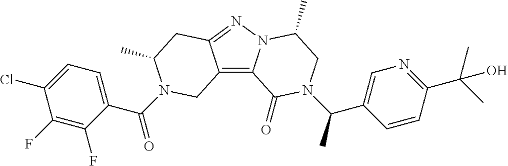 [SMILES: C[C@@H]1Cc2nn3c(c2CN1C(=O)c1ccc(Cl)c(F)c1F)C(=O)N([C@H](C)c1ccc(C(C)(C)O)nc1)C[C@H]3C]

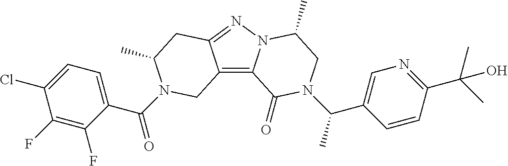 [SMILES: C[C@@H]1Cc2nn3c(c2CN1C(=O)c1ccc(Cl)c(F)c1F)C(=O)N([C@@H](C)c1ccc(C(C)(C)O)nc1)C[C@H]3C]

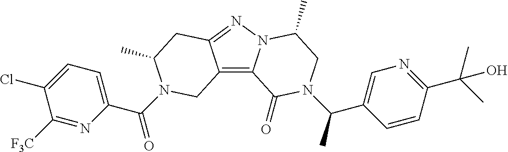 [SMILES: C[C@@H]1Cc2nn3c(c2CN1C(=O)c1ccc(Cl)c(C(F)(F)F)n1)C(=O)N([C@H](C)c1ccc(C(C)(C)O)nc1)C[C@H]3C]

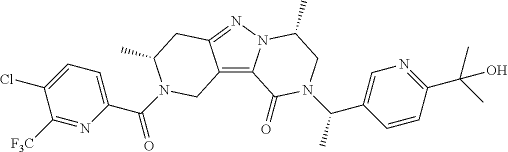 [SMILES: C[C@@H]1Cc2nn3c(c2CN1C(=O)c1ccc(Cl)c(C(F)(F)F)n1)C(=O)N([C@@H](C)c1ccc(C(C)(C)O)nc1)C[C@H]3C]

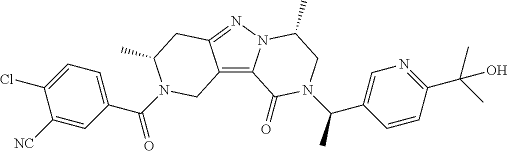 [SMILES: C[C@@H]1Cc2nn3c(c2CN1C(=O)c1ccc(Cl)c(C#N)c1)C(=O)N([C@H](C)c1ccc(C(C)(C)O)nc1)C[C@H]3C]

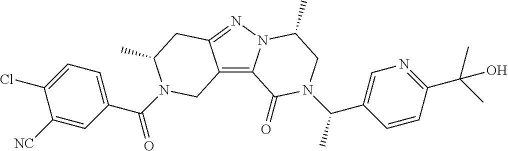 [SMILES: C[C@@H]1Cc2nn3c(c2CN1C(=O)c1ccc(Cl)c(C#N)c1)C(=O)N([C@@H](C)c1ccc(C(C)(C)O)nc1)C[C@H]3C]

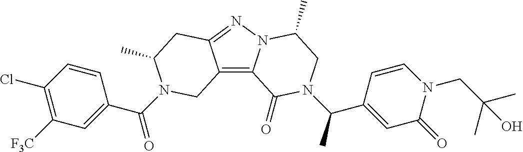 [SMILES: C[C@@H]1Cc2nn3c(c2CN1C(=O)c1ccc(Cl)c(C(F)(F)F)c1)C(=O)N([C@H](C)c1ccn(CC(C)(C)O)c(=O)c1)C[C@H]3C]

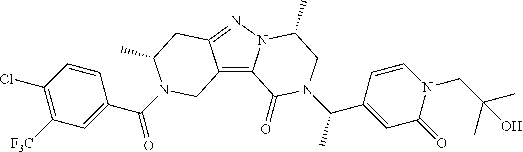 [SMILES: C[C@@H]1Cc2nn3c(c2CN1C(=O)c1ccc(Cl)c(C(F)(F)F)c1)C(=O)N([C@@H](C)c1ccn(CC(C)(C)O)c(=O)c1)C[C@H]3C]

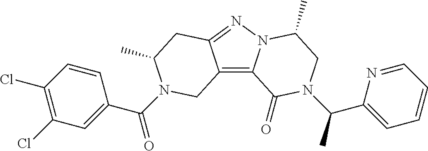 [SMILES: C[C@@H]1Cc2nn3c(c2CN1C(=O)c1ccc(Cl)c(Cl)c1)C(=O)N([C@H](C)c1ccccn1)C[C@H]3C]

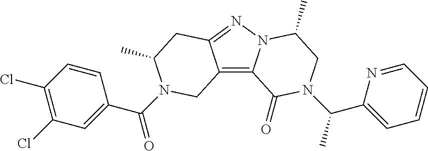 [SMILES: C[C@@H]1Cc2nn3c(c2CN1C(=O)c1ccc(Cl)c(Cl)c1)C(=O)N([C@@H](C)c1ccccn1)C[C@H]3C]

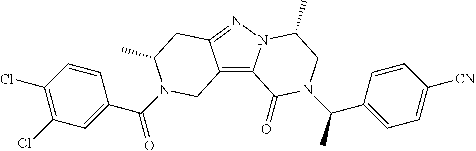 [SMILES: C[C@@H]1Cc2nn3c(c2CN1C(=O)c1ccc(Cl)c(Cl)c1)C(=O)N([C@H](C)c1ccc(C#N)cc1)C[C@H]3C]

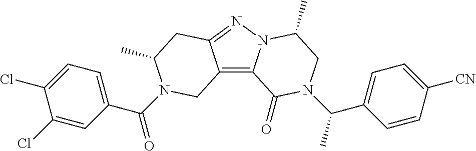 [SMILES: C[C@@H]1Cc2nn3c(c2CN1C(=O)c1ccc(Cl)c(Cl)c1)C(=O)N([C@@H](C)c1ccc(C#N)cc1)C[C@H]3C]

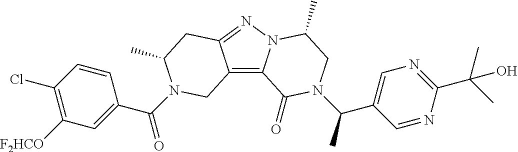 [SMILES: C[C@@H]1Cc2nn3c(c2CN1C(=O)c1ccc(Cl)c(OC(F)F)c1)C(=O)N([C@H](C)c1cnc(C(C)(C)O)nc1)C[C@H]3C]

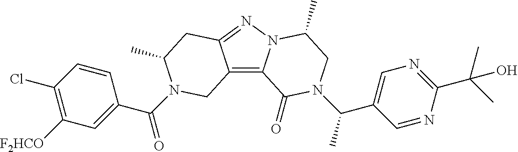 [SMILES: C[C@@H]1Cc2nn3c(c2CN1C(=O)c1ccc(Cl)c(OC(F)F)c1)C(=O)N([C@@H](C)c1cnc(C(C)(C)O)nc1)C[C@H]3C]

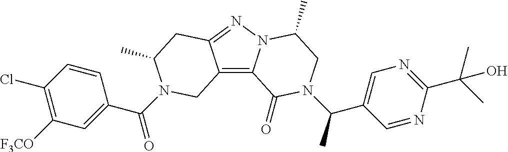 [SMILES: C[C@@H]1Cc2nn3c(c2CN1C(=O)c1ccc(Cl)c(OC(F)(F)F)c1)C(=O)N([C@H](C)c1cnc(C(C)(C)O)nc1)C[C@H]3C]

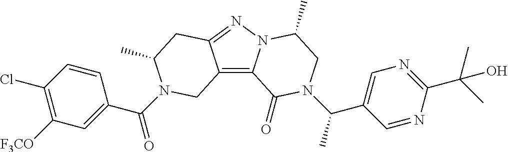 [SMILES: C[C@@H]1Cc2nn3c(c2CN1C(=O)c1ccc(Cl)c(OC(F)(F)F)c1)C(=O)N([C@@H](C)c1cnc(C(C)(C)O)nc1)C[C@H]3C]

### Pharmaceutical Compositions

Also disclosed herein are pharmaceutical compositions comprising
(A) at least one compound of any one of Formula (I), in any one of the embodiments defined above, or a pharmaceutically acceptable salt thereof, and
(B) at least one pharmaceutically acceptable excipient.

In embodiments, the pharmaceutical composition comprises at least one additional active or therapeutic agent. Additional active therapeutic agents may include, for example, an anti-HBV agent such as an HBV polymerase inhibitor, interferon, viral entry inhibitor, viral maturation inhibitor, capsid assembly modulator, reverse transcriptase inhibitor, immunomodulatory agent such as a TLR-agonist, or any other agents that affect the HBV life cycle and/or the consequences of HBV infection. The active agents of the present disclosure are used, alone or in combination with one or more additional active agents, to formulate pharmaceutical compositions of the present disclosure.

As used herein, the term “composition” or “pharmaceutical composition” refers to a mixture of at least one compound useful within the present disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the present disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the present disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure and are physiologically acceptable to the patient. Supplementary active compounds may also be incorporated into the compositions. Other additional ingredients that may be included in the pharmaceutical compositions used in the practice of the present disclosure are known in the art and described, for example in Remington's Pharmaceutical Sciences (Genaro, Ed., Mack Publishing Co., 1985, Easton, PA), which is incorporated herein by reference.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the active agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the present disclosure can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Oral tablets may include a compound according to the present disclosure mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract or may be coated with an enteric coating.

Capsules for oral administration include hard and soft gelatin capsules. To prepare hard gelatin capsules, compounds of the present disclosure may be mixed with a solid, semi-solid, or liquid diluent. Soft gelatin capsules may be prepared by mixing the compound of the present disclosure with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The active agents of this present disclosure may also be administered by non-oral routes. For example, the compositions may be formulated for rectal administration as a suppository. For parenteral use, including intravenous, intramuscular, intraperitoneal, or subcutaneous routes, the compounds of the present disclosure may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the present disclosure may utilize a patch formulation to affect transdermal delivery.

Compounds of the present disclosure may alternatively be administered in methods of this present disclosure by inhalation, via the nasal or oral routes, e.g., in a spray formulation also containing a suitable carrier.

### Methods of Use

The disclosed compounds are useful in the prevention or treatment of an HBV infection or of an HBV-induced disease in mammal in need thereof, more particularly in a human in need thereof.

In a non-limiting aspect, these compounds may (i) modulate or disrupt HBV assembly and other HBV core protein functions necessary for HBV replication or the generation of infectious particles, (ii) inhibit the production of infectious virus particles or infection, or (iii) interact with HBV capsid to effect defective viral particles with reduced infectivity or replication capacity acting as capsid assembly modulators. In particular, and without being bound to any particular mechanism of action, it is believed that the disclosed compounds are useful in HBV treatment by disrupting, accelerating, reducing, delaying and/or inhibiting normal viral capsid assembly and/or disassembly of immature or mature particles, thereby inducing aberrant capsid morphology leading to antiviral effects such as disruption of virion assembly and/or disassembly, virion maturation, virus egress and/or infection of target cells. The disclosed compounds may act as a disruptor of capsid assembly interacting with mature or immature viral capsid to perturb the stability of the capsid, thus affecting its assembly and/or disassembly. The disclosed compounds may perturb protein folding and/or salt bridges required for stability, function and/or normal morphology of the viral capsid, thereby disrupting and/or accelerating capsid assembly and/or disassembly. The disclosed compounds may bind capsid and alter metabolism of cellular polyproteins and precursors, leading to abnormal accumulation of protein monomers and/or oligomers and/or abnormal particles, which causes cellular toxicity and death of infected cells. The disclosed compounds may cause failure of the formation of capsids of optimal stability, affecting efficient uncoating and/or disassembly of viruses (e.g., during infectivity). The disclosed compounds may disrupt and/or accelerate capsid assembly and/or disassembly when the capsid protein is immature. The disclosed compounds may disrupt and/or accelerate capsid assembly and/or disassembly when the capsid protein is mature. The disclosed compounds may disrupt and/or accelerate capsid assembly and/or disassembly during viral infectivity which may further attenuate HBV viral infectivity and/or reduce viral load. The disruption, acceleration, inhibition, delay and/or reduction of capsid assembly and/or disassembly by the disclosed compounds may eradicate the virus from the host organism. Eradication of HBV from a subject by the disclosed compounds advantageously obviates the need for chronic long-term therapy and/or reduces the duration of long-term therapy.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

Additionally, HBV acts as a helper virus to hepatitis delta virus (HDV), and it is estimated that more than 15 million people may be HBV/HDV co-infected worldwide, with an increased risk of rapid progression to cirrhosis and increased hepatic decompensation, than patients suffering from HBV alone (Hughes, S. A. et al. Lancet 2011, 378, 73-85). HDV, infects therefore subjects suffering from HBV infection. In a particular embodiment, the compounds of the present disclosure may be used in the treatment and/or prophylaxis of HBV/HDV co-infection, or diseases associated with HBV/HDV co infection. Therefore, in a particular embodiment, the HBV infection is in particular HBV/HDV co-infection, and the mammal, in particular the human, may be HBV/HDV co-infected, or be at risk of HBV/HDV co infection.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In embodiments, the disclosed compounds are suitable for monotherapy. In embodiments, the disclosed compounds are effective against natural or native HBV strains. In embodiments, the disclosed compounds are effective against HBV strains resistant to currently known drugs.

In another embodiment, the compounds provided herein can be used in methods of modulating (e.g., inhibiting or disrupting) the activity, stability, function, and viral replication properties of HBV cccDNA.

In yet another embodiment, the compounds of the present disclosure can be used in methods of diminishing or preventing the formation of HBV cccDNA.

In another embodiment, the compounds provided herein can be used in methods of modulating (e.g., inhibiting or disrupting) the activity of HBV cccDNA.

In yet another embodiment, the compounds of the present disclosure can be used in methods of diminishing the formation of HBV cccDNA.

In another embodiment, the disclosed compounds can be used in methods of modulating, inhibiting, or disrupting the generation or release of HBV RNA particles from within the infected cell.

In a further embodiment, the total burden (or concentration) of HBV RNA particles is modulated. In a preferred embodiment, the total burden of HBV RNA is diminished.

In another embodiment, the methods provided herein reduce the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In another embodiment, the methods provided herein cause a lower incidence of viral mutation and/or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In another embodiment, the methods provided herein further comprise administering to the individual at least one HBV vaccine, a nucleoside HBV inhibitor, an interferon or any combination thereof.

In an aspect, provided herein is a method of treating an HBV infection in an individual in need thereof, comprising reducing the HBV viral load by administering to the individual a therapeutically effective amount of a compound of Formula (I), or a pharmaceutically acceptable salt thereof, alone or in combination with a reverse transcriptase inhibitor; and further administering to the individual a therapeutically effective amount of HBV vaccine.

An additional embodiment of the present disclosure is a method of treating a subject suffering from an HBV infection, comprising administering to a subject in need of such treatment an effective amount of at least one compound of Formula (I).

In another aspect, provided herein is a method of reducing the viral load associated with an HBV infection in an individual in need thereof, comprising administering to the individual a therapeutically effective amount of a compound of Formula (I), or a pharmaceutically acceptable salt thereof.

In another aspect, provided herein is a method of reducing reoccurrence of an HBV infection in an individual in need thereof, comprising administering to the individual a therapeutically effective amount of a compound of Formula (I), or a pharmaceutically acceptable salt thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In another aspect, provided herein is a method of reducing an adverse physiological impact of an HBV infection in an individual in need thereof, comprising administering to the individual a therapeutically effective amount of a compound of Formula (I), or a pharmaceutically acceptable salt thereof.

In another aspect, provided herein is a method of inducing remission of hepatic injury from an HBV infection in an individual in need thereof, comprising administering to the individual a therapeutically effective amount of a compound of Formula (I), or a pharmaceutically acceptable salt thereof.

In another aspect, provided herein is a method of reducing the physiological impact of long-term antiviral therapy for HBV infection in an individual in need thereof, comprising administering to the individual a therapeutically effective amount of a compound of Formula (I), or a pharmaceutically acceptable salt thereof.

In another aspect, provided herein is a method of prophylactically treating an HBV infection in an individual in need thereof, wherein the individual is afflicted with a latent HBV infection, comprising administering to the individual a therapeutically effective amount of a compound of Formula (I), or a pharmaceutically acceptable salt thereof.

In an embodiment, the methods provided herein further comprise monitoring the HBV viral load of the subject, wherein the method is carried out for a period of time such that the HBV virus is undetectable.

### Combinations

Provided herein are combinations of one or more of the disclosed compounds with at least one additional therapeutic agent. In embodiments, the methods provided herein can further comprise administering to the individual at least one additional therapeutic agent. In embodiments, the disclosed compounds are suitable for use in combination therapy. The compounds of the present disclosure may be useful in combination with one or more additional compounds useful for treating HBV infection. These additional compounds may comprise compounds of the present disclosure or compounds known to treat, prevent, or reduce the symptoms or effects of HBV infection.

In an exemplary embodiment, additional active ingredients are those that are known or discovered to be effective in the treatment of conditions or disorders involved in HBV infection, such as another HBV capsid assembly modulator or a compound active against another target associated with the particular condition or disorder involved in HBV infection, or the HBV infection itself. The combination may serve to increase efficacy (e.g., by including in the combination a compound potentiating the potency or effectiveness of an active agent according to the present disclosure), decrease one or more side effects, or decrease the required dose of the active agent according to the present disclosure. In a further embodiment, the methods provided herein allow for administering of the at least one additional therapeutic agent at a lower dose or frequency as compared to the administering of the at least one additional therapeutic agent alone that is required to achieve similar results in prophylactically treating an HBV infection in an individual in need thereof.

Such compounds include but are not limited to HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and any other agent that affects the HBV life cycle and/or affect the consequences of HBV infection or combinations thereof.

In embodiments, the compounds of the present disclosure may be used in combination with an HBV polymerase inhibitor, immunomodulatory agents, interferon such as pegylated interferon, viral entry inhibitor, viral maturation inhibitor, capsid assembly modulator, reverse transcriptase inhibitor, a cyclophilin/TNF inhibitor, immunomodulatory agent such as a TLR-agonist, an HBV vaccine, and any other agent that affects the HBV life cycle and/or affect the consequences of HBV infection or combinations thereof. In particular, the compounds of the present disclosure may be used in combination with one or more agents (or a salt thereof) selected from the group consisting of

HBV reverse transcriptase inhibitors, and DNA and RNA polymerase inhibitors, including but not limited to: lamivudine (3TC, Zeffix, Heptovir, Epivir, and Epivir-HBV), entecavir (Baraclude, Entavir), adefovir dipivoxil (Hepsara, Preveon, bis-POM PMEA), tenofovir disoproxil fumarate (Viread, TDF or PMPA);

interferons, including but not limited to interferon alpha (IFN-α), interferon beta (IFN-β), interferon lambda (IFN-λ), and interferon gamma (IFN-γ);

viral entry inhibitors;

viral maturation inhibitors;

literature-described capsid assembly modulators, such as, but not limited to BAY 41-4109;

reverse transcriptase inhibitor;

an immunomodulatory agent such as a TLR-agonist; and

agents of distinct or unknown mechanism, such as but not limited to AT-61 ((E)-N-(1-chloro-3-oxo-1-phenyl-3-(piperidin-1-yl)prop-1-en-2-yl)benzamide), AT-130 ((E)-N-(1-bromo-1-(2-methoxyphenyl)-3-oxo-3-(piperidin-1-yl)prop-1-en-2-yl)-4-nitrobenzamide), and similar analogs.

In embodiments, the additional therapeutic agent is an interferon. The term “interferon” or “IFN” refers to any member the family of highly homologous species-specific proteins that inhibit viral replication and cellular proliferation and modulate immune response. Human interferons are grouped into three classes; Type I, which include interferon-alpha (IFN-α), interferon-beta (IFN-β), and interferon-omega (IFN-ω), Type II, which includes interferon-gamma (IFN-γ), and Type III, which includes interferon-lambda (IFN-λ). Recombinant forms of interferons that have been developed and are commercially available are encompassed by the term “interferon” as used herein. Subtypes of interferons, such as chemically modified or mutated interferons, are also encompassed by the term “interferon” as used herein. Chemically modified interferons include pegylated interferons and glycosylated interferons. Examples of interferons also include, but are not limited to, interferon-alpha-2a, interferon-alpha-2b, interferon-alpha-n1, interferon-beta-1a, interferon-beta-1b, interferon-lamda-1, interferon-lamda-2, and interferon-lamda-3. Examples of pegylated interferons include pegylated interferon-alpha-2a and pegylated interferon alpha-2b.

Accordingly, in one embodiment, the compounds of Formula I, can be administered in combination with an interferon selected from the group consisting of interferon alpha (IFN-α), interferon beta (IFN-β), interferon lambda (IFN-λ), and interferon gamma (IFN-γ). In one specific embodiment, the interferon is interferon-alpha-2a, interferon-alpha-2b, or interferon-alpha-n1. In another specific embodiment, the interferon-alpha-2a or interferon-alpha-2b is pegylated. In a preferred embodiment, the interferon-alpha-2a is pegylated interferon-alpha-2a (PEGASYS).

In another embodiment, the additional therapeutic agent is selected from immune modulator or immune stimulator therapies, which includes biological agents belonging to the interferon class.

Further, the additional therapeutic agent may be an agent that disrupts the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors. In a further embodiment of the combination therapy, the reverse transcriptase inhibitor and/or DNA and/or RNA polymerase inhibitor is Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

In an embodiment, the additional therapeutic agent is an immunomodulatory agent that induces a natural, limited immune response leading to induction of immune responses against unrelated viruses. In other words, the immunomodulatory agent can affect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist. In further embodiment of the combination therapy, the TLR-7 agonist is selected from the group consisting of SM360320 (9-benzyl-8-hydroxy-2-(2-methoxy-ethoxy)adenine) and AZD 8848 (methyl [3-({[3-(6-amino-2-butoxy-8-oxo-7,8-dihydro-9H-purin-9-yl)propyl][3-(4-morpholinyl)propyl]amino}methyl)phenyl]acetate).

In any of the methods provided herein, the method may further comprise administering to the individual at least one HBV vaccine, a nucleoside HBV inhibitor, an interferon or any combination thereof. In an embodiment, the HBV vaccine is at least one of RECOMBIVAX HB, ENGERIX-B, ELOVAC B, GENEVAC-B, or SHANVAC B.

In another aspect, provided herein is method of treating an HBV infection in an individual in need thereof, comprising reducing the HBV viral load by administering to the individual a therapeutically effective amount of a compound of the present disclosure alone or in combination with a reverse transcriptase inhibitor; and further administering to the individual a therapeutically effective amount of HBV vaccine. The reverse transcriptase inhibitor may be one of Zidovudine, Didanosine, Zalcitabine, ddA, Stavudine, Lamivudine, Abacavir, Emtricitabine, Entecavir, Apricitabine, Atevirapine, ribavirin, acyclovir, famciclovir, valacyclovir, ganciclovir, valganciclovir, Tenofovir, Adefovir, PMPA, cidofovir, Efavirenz, Nevirapine, Delavirdine, or Etravirine.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Beneficial Effects

The present compounds have improved human liver microsome stability as well as reasonable anti-HBV activity. As compared to comparative compounds, the half-life (t_{1/2}) of the present compounds are significantly increased, showing great improvement in human metabolic stability and the advantages in pharmaceutical applications.

### Methods

The present disclosure relates to a method for the preparation of a compound of Formula (I) as described herein.

In an exemplary embodiment, the method may comprise the steps of:
1) reacting a compound of Formula (a)

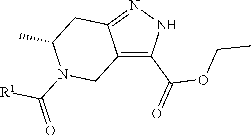

and a compound of Formula (b)

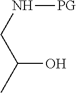

(wherein PG is a protecting group) to give a compound of Formula (c)

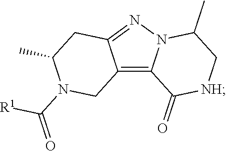

2) reacting the compound of Formula (c) with a compound of Formula (d)

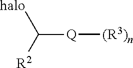

to give a compound of Formula (e)

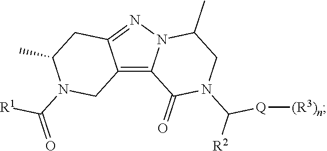

3) subjecting the compound of Formula (e) to chiral separation to give the compound of Formula (I);

wherein R¹, R², R³, Q, halo, and n are as defined herein.

As the product of step 2) (compound of Formula (e)) may have one or more chiral centers, in step 3) one or more chiral separation may be performed to give individual enantiopure compound.

In an embodiment, in step 1), the reaction product of the compound of Formula (a) and the compound of Formula (b) is subjected to deprotection and cyclization to give the compound of Formula (c).

In step 1), a compound of Formula (b)

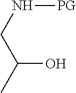

with desirable chiral structure (for example, Formula (b′)

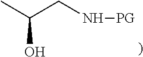

may be used such that the product of step 1) may have desirable chiral structure as well (for example, Formula (c′)

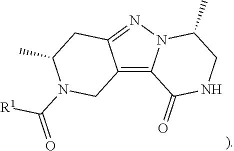

Likewise, the product of step 2) may have desirable chiral structure as well (for example, Formula (e′)

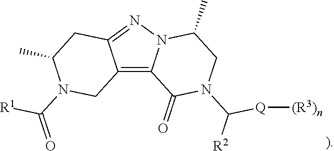

PG is a protecting group, which is conventionally used and is preferably Boc or Cbz.

An exemplary scheme is as follows.

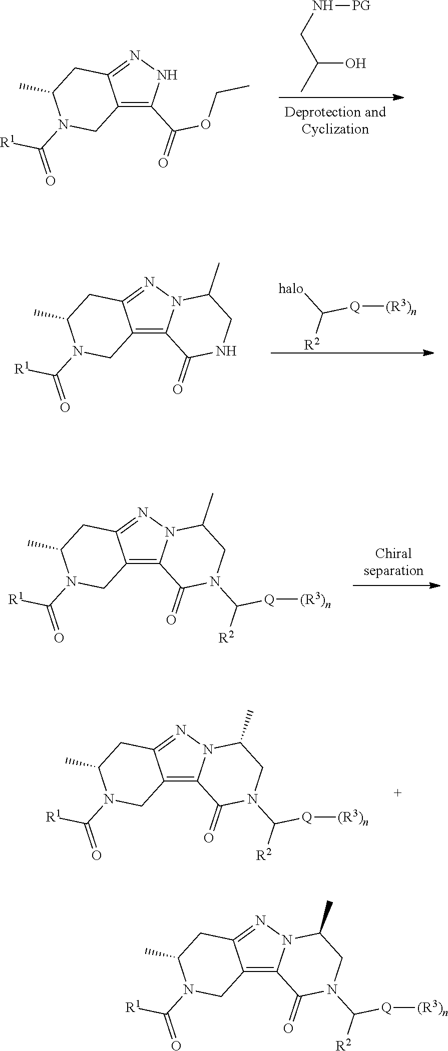

In an alternative embodiment, the method may comprise the steps of:
1) reacting a compound of Formula (f)

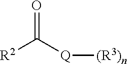

and Formula (b-1)

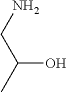

with introduction of a protecting group (PG) to give a compound of Formula (g)

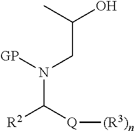

(wherein PG is a protecting group);
2) reacting a compound of Formula (a)

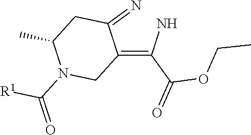

and a compound of Formula (g)

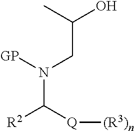

to give a compound of Formula (e)

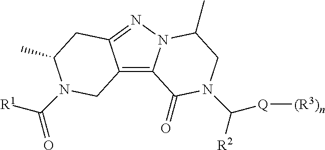

3) subjecting the compound of Formula (e) to chiral separation to give the compound of Formula (I),

wherein R¹, R², R³, Q, halo, and n are as defined herein.

As the product of step 2) (compound of Formula (e)) may have one or more chiral centers, in step 3) one or more chiral separation may be performed to give individual enantiopure compound.

In an embodiment, in step 2), the reaction product of the compound of Formula (a) and the compound of Formula (g) is subjected to hydrolysis, deprotection and cyclization to give the compound of Formula (e).

In step 1), a compound of Formula (b-1)

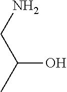

with desirable chiral structure (for example, Formula (b-1′)

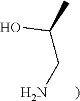

may be used such that the product of step 1) may have desirable chiral structure as well (for example, Formula (g′)

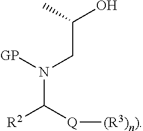

Likewise, the product of step 2) may have desirable chiral structure as well (for example, Formula (e′)

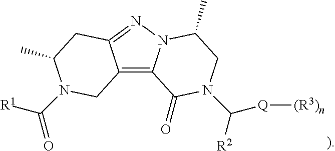

PG is a protecting group, which is conventionally used and is preferably Boc or Cbz.

An exemplary scheme is as follows.

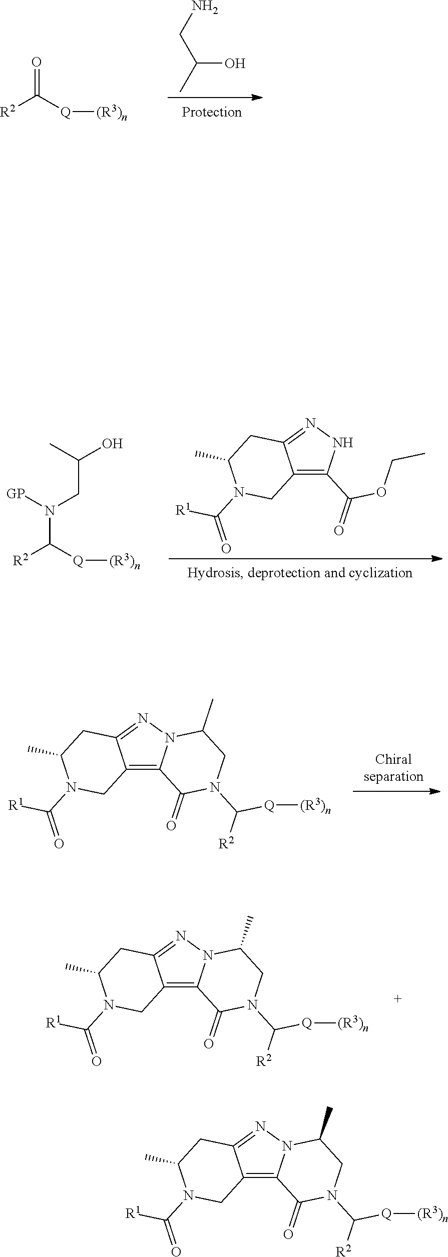

### Definitions

Listed below are definitions of various terms used to describe this present disclosure. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the applicable art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include,” “includes,” and “included,” is not limiting.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds. All ranges disclosed herein are inclusive of the recited endpoint and independently combinable (for example, the range of “from 50 mg to 300 mg” is inclusive of the endpoints, 50 mg and 300 mg, and all the intermediate values). The endpoints of the ranges and any values disclosed herein are not limited to the precise range or value; they are sufficiently imprecise to include values approximating these ranges and/or values.

As used herein, approximating language can be applied to modify any quantitative representation that can vary without resulting in a change in the basic function to which it is related. Accordingly, a value modified by a term or terms, such as “substantially,” cannot be limited to the precise value specified, in some cases. In at least some instances, the approximating language can correspond to the precision of an instrument for measuring the value.

As used herein, the term “at least one” or “one or more” refers to one, two, three, four, five, six, seven, eight, nine or more.

The term “alkyl” as a group or as part of another group, refers to a straight- or branched-chain alkyl group having carbon and hydrogen atoms in the chain. Examples of alkyl groups include methyl (Me, which also may be structurally depicted by the symbol, “/”), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples. The term C₁₋₄alkyl as used here refers to a straight- or branched-chain alkyl group having from 1 to 4 carbon atoms in the chain. The term C₁₋₆alkyl as used here refers to a straight- or branched-chain alkyl group having from 1 to 6 carbon atoms in the chain.

The term “alkoxyl” as a group or as part of another group, refers to an alkyl group which is linked to the rest of the molecule via an oxygen, wherein the alkyl is as defined herein. The term C₁₋₄alkoxyl as used here refers to a straight- or branched-chain alkoxyl group having from 1 to 4 carbon atoms in the chain. The term C₁₋₆ alkoxyl as used here refers to a straight- or branched-chain alkoxyl group having from 1 to 6 carbon atoms in the chain. Examples of alkoxyl groups include methoxy, ethoxy, propoxy, butoxy, pentyloxy, hexyloxy, and groups that in light of the ordinary skill in the art and the teachings provided herein would be considered equivalent to any one of the foregoing examples.

The term “C₃₋₆cycloalkyl” refers to a saturated monocyclic carbocycle having from 3 to 6 ring atoms. Illustrative examples of cycloalkyl groups include cyclopropyl, cyclobutyl, cyclopentyl, and cyclohexyl.

The term “phenyl” represents the following moiety:

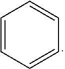

The term “heteroaryl” used herein refers to an aromatic monocyclic or bicyclic aromatic ring system having 5 to 10 ring members and which contains carbon atoms and from 1 to 4 heteroatoms independently selected from the group consisting of N, O, and S. Included within the term heteroaryl are aromatic rings of 5 or 6 members wherein the ring consists of carbon atoms and has at least one (e.g., 1, 2 or 3, preferably 1 or 2) heteroatom member. Suitable heteroatoms include nitrogen (N), oxygen (S), and sulfur (S), preferably nitrogen (N). In the case of 5 membered rings, the heteroaryl ring preferably contains one member of nitrogen, oxygen or sulfur and, in addition, up to 3 additional nitrogens. In the case of 6 membered rings, the heteroaryl ring preferably contains from 1 to 4, e.g. tetrazolyl, more in particular from 1 to 3 nitrogen atoms. For the case wherein the 6 membered ring has 3 nitrogens, at most 2 nitrogen atoms are adjacent. Examples of heteroaryl groups include but not limited to furyl, thienyl, pyrrolyl, oxazolyl, thiazolyl, imidazolyl, pyrazolyl, oxazolyl, thiazolyl, oxadiazolyl, triazolyl, thiadiazolyl, pyridinyl (pyridyl), pyridazinyl, pyrimidinyl, pyrazinyl, indolyl, isoindolyl, benzofuryl, benzothienyl, indazolyl, benzimidazolyl, benzothiazolyl, benzoxazolyl, benzisoxazolyl, benzothiadiazolyl, benzotriazolyl, quinolinyl, isoquinolinyl and quinazolinyl. Unless otherwise noted, the heteroaryl is attached to its pendant group at any heteroatom or carbon atom that results in a stable structure.

Those skilled in the art will recognize that the species of heteroaryl groups listed or illustrated above are not exhaustive, and that additional species within the scope of these defined terms may also be selected.

The term “heterocyclyl” represents a non-aromatic monocyclic or bicyclic system, unless otherwise specified, having for example, 4 to 8 ring members, more usually 5 to 6 ring members. Examples of monocyclic groups are groups containing 4 to 8 ring members, more usually, 5 or 6 ring members. Non-limiting examples of monocyclic heterocyclyl systems containing at least one heteroatom selected from nitrogen, oxygen or sulfur (N, O, S) include, but are not limited to 4- to 8-membered heterocyclyl systems such as oxetanyl, pyrrolidinyl, tetrahydrofuranyl, piperidinyl, piperazinyl, pyranyl, dihydropyranyl, tetrahydropyranyl, morpholinyl, thiomorpholinyl. Unless otherwise specified, each can be bound to the remainder of the molecule through any available ring carbon atom or nitrogen atom, and may optionally be substituted, where possible, on carbon and/or nitrogen atoms according to the embodiments. Optional substituents of 4- to 8-membered monocyclic heterocyclyl, include oxo, OH, OC₁₋₄ alkyl, halo, COOH, CONHCH₃, NHCOC₁₋₄alkyl, NHCOC₃₋₆cycloalkyl, and C₁₋₄alkyl.

The term “cyano” refers to the group —CN.

The terms “halo” or “halogen” represent chloro (Cl), fluoro (F), bromo (Br) or iodo (I).

The term “oxo” represents ═O.

The term “hydroxyl” represents —OH.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

The terms “para”, “meta”, and “ortho” have the meanings as understood in the art. Thus, for example, a fully substituted phenyl group has substituents at both “ortho” (o) positions adjacent to the point of attachment of the phenyl ring, both “meta” (m) positions, and the one “para” (p) position across from the point of attachment. To further clarify the position of substituents on the phenyl ring, the 2 different ortho positions will be designated as ortho and ortho′ and the 2 different meta positions as meta and meta′ as illustrated below.

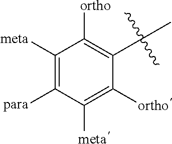

When referring to substituents on a pyridyl group, the terms “para”, “meta”, and “ortho” refer to the placement of a substituent relative to the point of attachment of the pyridyl ring. For example, the structure below is described as 3-pyridyl with the X¹ substituent in the ortho position, the X² substituent in the meta position, and X³ substituent in the para position:

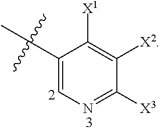

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. Whenever a yield is given as a percentage, such yield refers to a mass of the entity for which the yield is given with respect to the maximum amount of the same entity that could be obtained under the particular stoichiometric conditions. Concentrations that are given as percentages refer to mass ratios, unless indicated differently.

The terms “buffered” solution or “buffer” solution are used herein interchangeably according to their standard meaning. Buffered solutions are used to control the pH of a medium, and their choice, use, and function is known to those of ordinary skill in the art. See, for example, G. D. Considine, ed., Van Nostrand's Encyclopedia of Chemistry, p. 261, 5^{th} ed. (2005), describing, inter alia, buffer solutions and how the concentrations of the buffer constituents relate to the pH of the buffer. For example, a buffered solution is obtained by adding MgSO₄ and NaHCO₃ to a solution in a 10:1 w/w ratio to maintain the pH of the solution at about 7.5.

Any formula given herein is intended to represent compounds having structures depicted by the structural formula as well as certain variations or forms. In particular, compounds of any formula given herein may have asymmetric centers and therefore exist in different enantiomeric forms. All optical isomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers.

It is also to be understood that compounds that have the same molecular formula but differ in the nature or sequence of bonding of their atoms or the arrangement of their atoms in space are termed “isomers.”

Stereoisomers that are not mirror images of one another are termed “diastereomers” and those that are non-superimposable mirror images of each other are termed “enantiomers.” When a compound has an asymmetric center, for example, it is bonded to four different groups, and a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+)- or (−)-isomers respectively). A chiral compound can exist as either an individual enantiomer or as a mixture thereof. A mixture containing equal proportions of the enantiomers is called a “racemic mixture.”

“Tautomers” refer to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenyl nitromethane, that are likewise formed by treatment with acid or base.

Tautomeric forms may be relevant to the attainment of the optimal chemical reactivity and biological activity of a compound of interest.

The compounds of this present disclosure may possess one or more asymmetric centers; such compounds can therefore be produced as individual (R)- or (S)-stereoisomers or as mixtures thereof.

Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art.

Certain examples contain chemical structures that are depicted as an absolute enantiomer but are intended to indicate enantiopure material that is of unknown configuration.

In these cases (R*) or (S*) or (*R) or (*S) is used in the name to indicate that the absolute stereochemistry of the corresponding stereocenter is unknown. Thus, a compound designated as (R*) or (*R) refers to an enantiopure compound with an absolute configuration of either (R) or (S). In cases where the absolute stereochemistry has been confirmed, the structures are named using (R) and (S).

[symbol]

The symbols [symbol] and [symbol] are used as meaning the same spatial arrangement in chemical structures shown herein. Analogously, the symbols [symbol] and [symbol] are used as meaning the same spatial arrangement in chemical structures shown herein.

Certain compounds of Formula (I), or pharmaceutically acceptable salts of compounds of Formula (I), may be obtained as solvates. Solvates include those formed from the interaction or complexation of compounds of the present disclosure with one or more solvents, either in solution or as a solid or crystalline form. In some embodiments, the solvent is water and the solvates are hydrates.

Reference to a compound herein stands for a reference to any one of: (a) the actually recited form of such compound, and (b) any of the forms of such compound in the medium in which the compound is being considered when named. For example, reference herein to a compound such as R—COOH, encompasses reference to any one of, for example, R—COOH₍ₛ₎, R—COOH₍ₛₒₗ₎, and R-COO⁻₍ₛₒₗ₎. In this example, R—COOH₍ₛ₎ refers to the solid compound, as it could be for example in a tablet or some other solid pharmaceutical composition or preparation; R—COOH₍ₛₒₗ₎ refers to the undissociated form of the compound in a solvent; and R—COO⁻₍ₛₒₗ₎ refers to the dissociated form of the compound in a solvent, such as the dissociated form of the compound in an aqueous environment, whether such dissociated form derives from R—COOH, from a salt thereof, or from any other entity that yields R—COO⁻ upon dissociation in the medium being considered. In another example, an expression such as “exposing an entity to compound of formula R—COOH” refers to the exposure of such entity to the form, or forms, of the compound R—COOH that exists, or exist, in the medium in which such exposure takes place. In still another example, an expression such as “reacting an entity with a compound of formula R—COOH” refers to the reacting of (a) such entity in the chemically relevant form, or forms, of such entity that exists, or exist, in the medium in which such reacting takes place, with (b) the chemically relevant form, or forms, of the compound R—COOH that exists, or exist, in the medium in which such reacting takes place. In this regard, if such entity is for example in an aqueous environment, it is understood that the compound R—COOH is in such same medium, and therefore the entity is being exposed to species such as R—COOH_{(aq)} and/or R-COO⁻_{(aq)}, where the subscript “(aq)” stands for “aqueous” according to its conventional meaning in chemistry and biochemistry. A carboxylic acid functional group has been chosen in these nomenclature examples; this choice is not intended, however, as a limitation but it is merely an illustration. It is understood that analogous examples can be provided in terms of other functional groups, including but not limited to hydroxyl, basic nitrogen members, such as those in amines, and any other group that interacts or transforms according to known manners in the medium that contains the compound. Such interactions and transformations include, but are not limited to, dissociation, association, tautomerism, solvolysis, including hydrolysis, solvation, including hydration, protonation, and deprotonation. No further examples in this regard are provided herein because these interactions and transformations in a given medium are known by any one of ordinary skill in the art.

In another example, a zwitterionic compound is encompassed herein by referring to a compound that is known to form a zwitterion, even if it is not explicitly named in its zwitterionic form. Terms such as zwitterion, zwitterions, and their synonyms zwitterionic compound(s) are standard IUPAC-endorsed names that are well known and part of standard sets of defined scientific names. In this regard, the name zwitterion is assigned the name identification CHEBI:27369 by the Chemical Entities of Biological Interest (ChEBI) dictionary of molecular entities. As generally well known, a zwitterion or zwitterionic compound is a neutral compound that has formal unit charges of opposite sign. Sometimes these compounds are referred to by the term “inner salts”. Other sources refer to these compounds as “dipolar ions”, although the latter term is regarded by still other sources as a misnomer. As a specific example, aminoethanoic acid (the amino acid glycine) has the formula H₂NCH₂COOH, and it exists in some media (in this case in neutral media) in the form of the zwitterion ⁺H₃NCH₂COO⁻. Zwitterions, zwitterionic compounds, inner salts and dipolar ions in the known and well established meanings of these terms are within the scope of this present disclosure, as would in any case be so appreciated by those of ordinary skill in the art. Because there is no need to name each and every embodiment that would be recognized by those of ordinary skill in the art, no structures of the zwitterionic compounds that are associated with the compounds of this present disclosure are given explicitly herein. They are, however, part of the embodiments of this present disclosure. No further examples in this regard are provided herein because the interactions and transformations in a given medium that lead to the various forms of a given compound are known by any one of ordinary skill in the art.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the present disclosure include isotopes of hydrogen, carbon, nitrogen, oxygen, phosphorus, sulfur, fluorine, chlorine, and iodine such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, ¹²⁵I, respectively. Such isotopically labeled compounds are useful in metabolic studies (preferably with ¹⁴C), reaction kinetic studies (with, for example deuterium (i.e., D or ²H); or tritium (i.e., T or ³H)), detection or imaging techniques such as positron emission tomography (PET) or single-photon emission computed tomography (SPECT) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this present disclosure can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the same choice of the species for the variable appearing elsewhere. In other words, where a variable appears more than once, the choice of the species from a specified list is independent of the choice of the species for the same variable elsewhere in the formula, unless stated otherwise.

According to the foregoing interpretive considerations on assignments and nomenclature, it is understood that explicit reference herein to a set implies, where chemically meaningful and unless indicated otherwise, independent reference to embodiments of such set, and reference to each and every one of the possible embodiments of subsets of the set referred to explicitly.

By way of a first example on substituent terminology, if substituent S¹ₑₓₐₘₚₗₑ is one of S₁ and S₂, and substituent S²ₑₓₐₘₚₗₑ is one of S₃ and S₄, then these assignments refer to embodiments of this present disclosure given according to the choices S¹ₑₓₐₘₚₗₑ is S₁ and S²ₑₓₐₘₚₗₑ is S₃; S¹ₑₓₐₘₚₗₑ is S₁ and S²ₑₓₐₘₚₗₑ is S₄; S¹ₑₓₐₘₚₗₑ is S₂ and S²ₑₓₐₘₚₗₑ is S₃; S¹ₑₓₐₘₚₗₑ is S₂ and S²ₑₓₐₘₚₗₑ is S₄; and equivalents of each one of such choices. The shorter terminology “S¹ₑₓₐₘₚₗₑ is one of S₁ and S₂, and S²ₑₓₐₘₚₗₑ is one of S₃ and S₄” is accordingly used herein for the sake of brevity, but not by way of limitation. The foregoing first example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

Furthermore, when more than one assignment is given for any member or substituent, embodiments of this present disclosure comprise the various groupings that can be made from the listed assignments, taken independently, and equivalents thereof. By way of a second example on substituent terminology, if it is herein described that substituent Sₑₓₐₘₚₗₑ is one of S₁, S₂, and S₃, this listing refers to embodiments of this present disclosure for which Sₑₓₐₘₚₗₑ is S₁; Sₑₓₐₘₚₗₑ is S₂; Sₑₓₐₘₚₗₑ is S₃; Sₑₓₐₘₚₗₑ is one of S₁ and S₂; Sₑₓₐₘₚₗₑ is one of S₁ and S₃; Sₑₓₐₘₚₗₑ is one of S₂ and S₃; Sₑₓₐₘₚₗₑ is one of S₁, S₂ and S₃; and Sₑₓₐₘₚₗₑ is any equivalent of each one of these choices. The shorter terminology “Sₑₓₐₘₚₗₑ is one of S₁, S₂, and S₃” is accordingly used herein for the sake of brevity, but not by way of limitation. The foregoing second example on substituent terminology, which is stated in generic terms, is meant to illustrate the various substituent assignments described herein. The foregoing convention given herein for substituents extends, when applicable, to members such as R¹, R², R³, R⁴, R⁵, G¹, G², G³, G⁴, G⁵, G⁶, G⁷, G⁸, G⁹, G¹⁰, G¹¹, n, L, R, T, Q, W, X, Y, and Z and any other generic substituent symbol used herein.

The nomenclature “Cᵢ₋ⱼ” with j>i, when applied herein to a class of substituents, is meant to refer to embodiments of this present disclosure for which each and every one of the number of carbon members, from i to j including i and j, is independently realized. By way of example, the term C₁₋₆ refers independently to embodiments that have one carbon member (C₁), embodiments that have two carbon members (C₂), embodiments that have three carbon members (C₃), embodiments that have four carbon members (C₄), embodiments that have five carbon members (C₅), and embodiments that have six carbon members (C₆).

The term Cₙ₋ₘalkyl refers to an aliphatic chain, whether straight or branched, with a total number N of carbon members in the chain that satisfies n≤N≤m, with m>n. Any disubstituent referred to herein is meant to encompass the various attachment possibilities when more than one of such possibilities are allowed. For example, reference to disubstituent -A-B-, where A≠B, refers herein to such disubstituent with A attached to a first substituted member and B attached to a second substituted member, and it also refers to such disubstituent with A attached to the second substituted member and B attached to the first substituted member.

The present disclosure includes also pharmaceutically acceptable salts of the compounds of Formula (I), preferably of those described above and of the specific compounds exemplified herein, and methods of treatment using such salts.

The term “pharmaceutically acceptable” means approved or approvable by a regulatory agency of Federal or a state government or the corresponding agency in countries other than the United States, or that is listed in the U. S. Pharmacopoeia or other generally recognized pharmacopoeia for use in animals, and more particularly, in humans.

A “pharmaceutically acceptable salt” is intended to mean a salt of a free acid or base of compounds represented by Formula (I) that are non-toxic, biologically tolerable, or otherwise biologically suitable for administration to the subject. It should possess the desired pharmacological activity of the parent compound. See, generally, G. S. Paulekuhn, et al., “Trends in Active Pharmaceutical Ingredient Salt Selection based on Analysis of the Orange Book Database”, J. Med. Chem., 2007, 50:6665-72, S. M. Berge, et al., “Pharmaceutical Salts”, J Pharm Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt.

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein within or to the patient such that it can perform its intended function. Typically, such constructs are carried or transported from one organ, or portion of the body, to another organ, or portion of the body. Each carrier must be “acceptable” in the sense of being compatible with the other ingredients of the formulation, including the compound provided herein, and not injurious to the patient. Some examples of materials that can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds can also be incorporated into the compositions. The “pharmaceutically acceptable carrier” can further include a pharmaceutically acceptable salt of the compound provided herein. Other additional ingredients that can be included in the pharmaceutical compositions provided herein are known in the art and described, for example in Remington's Pharmaceutical Sciences (Genaro, Ed., Mack Publishing Co., 1985, Easton, PA), which is incorporated herein by reference.

The term “stabilizer,” as used herein, refers to polymers capable of chemically inhibiting or preventing degradation of a compound disclosed herein. Stabilizers are added to formulations of compounds to improve chemical and physical stability of the compound.

The term “tablet,” as used herein, denotes an orally administrable, single-dose, solid dosage form that can be produced by compressing a drug substance or a pharmaceutically acceptable salt thereof, with suitable excipients (e.g., fillers, disintegrants, lubricants, glidants, and/or surfactants) by conventional tableting processes.

As used herein, the term “capsule” refers to a solid dosage form in which the drug is enclosed within either a hard or soft soluble container or “shell.” The container or shell can be formed from gelatin, starch and/or other suitable substances.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

The term “combination,” “therapeutic combination,” “pharmaceutical combination,” or “combination product” as used herein refer to a non-fixed combination or a kit of parts for the combined administration where two or more therapeutic agents can be administered independently, at the same time or separately within time intervals, especially where these time intervals allow that the combination partners show a cooperative, e.g., synergistic, effect.

The term “modulators” include both inhibitors and activators, where “inhibitors” refer to compounds that decrease, prevent, inactivate, desensitize, or down-regulate HBV assembly and other HBV core protein functions necessary for HBV replication or the generation of infectious particles.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies and/or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity and/or is lethal to the virus.

As used herein, the term “treatment” or “treating,” is defined as the application or administration of a therapeutic agent, i.e., a compound of the present disclosure (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject or individual is human.

In treatment methods according to the present disclosure, an effective amount of a pharmaceutical agent according to the present disclosure is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present disclosure may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day. An example of a dose of a compound is from about 1 mg to about 2,500 mg.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

HBV infections that may be treated according to the disclosed methods include HBV genotype A, B, C, and/or D infections. However, in an embodiment, the methods disclosed may treat any HBV genotype (“pan-genotypic treatment”). HBV genotyping may be performed using methods known in the art, for example, INNO-LIPA® HBV Genotyping, Innogenetics N.V., Ghent, Belgium).

Some exemplary embodiments are provided as follows.

Embodiment 1. A compound of Formula (I),

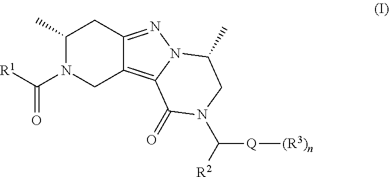

or a stereoisomeric or a tautomeric form thereof, wherein

R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃;

R² is selected from the group consisting of H, CHF₂, CF₃, C₁₋₆alkyl, C₁₋₆alkylOC₁₋₆alkyl and C₃₋₆cycloalkyl;

Q represents a ring selected from the group consisting of phenyl, 5-membered heteroaryl and 6-membered heteroaryl;

n represents 1, 2 or 3;

each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl and halo, each of C₁₋₆alkyl, C₁₋₆alkoxyl, C₃₋₆cycloalkyl, 5-membered heteroaryl, 6-membered heteroaryl and 4-8 membered heterocyclyl is optionally substituted with 1, 2, 3, 4 or 5 substituents, each of said substituents independently selected from the group consisting of halo, hydroxyl, C₁₋₆alkyl and oxo;

or a pharmaceutically acceptable salt or a solvate thereof.

Embodiment 2. The compound of Embodiment 1, wherein R¹ is a ring selected from the group consisting of phenyl, pyridyl, pyrimidinyl, pyridazinyl and pyrazinyl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃.

Embodiment 3. The compound of any one of the preceding Embodiments, wherein R¹ is phenyl, which is substituted with 1, 2 or 3 substituents, each of said substituents independently selected from the group consisting of halo and CN.

Embodiment 4. The compound of any one of the preceding Embodiments, wherein the structural unit

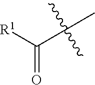

in Formula (I) satisfies Formula (Ia)

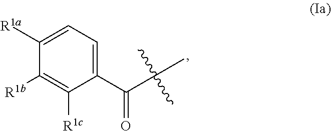

wherein R^{1a}, R^{1b}, and R^{1c}, each independently are selected from the group consisting of hydrogen, halo, C₁₋₆alkyl, C₃₋₆cycloalkyl, CN, CF₃, CHF₂, OCHF₂ and OCF₃, with at least one of R^{1a}, R^{1b}, and R^{1c} not being hydrogen.

Embodiment 5. The compound of Embodiment 4, wherein R^{1a} is halo, R^{1b} is selected from the group consisting of halo and cyano, and wherein R^{1c} is hydrogen.

Embodiment 6. The compound of any one of the preceding Embodiments, wherein

R² is selected from the group consisting of CHF₂, CF₃, C₁₋₆alkyl, C₁₋₆alkylOC₁₋₆alkyl and C₃₋₆cycloalkyl; and

the structure of Formula (I) has Formula (I-1) or Formula (I-2)

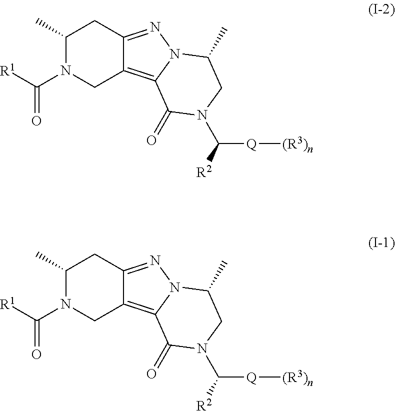

Embodiment 7. The compound of any one of the preceding Embodiments, wherein R² is methyl.

Embodiment 8. The compound of any one of the preceding Embodiments, wherein Q is a ring selected from the group consisting of phenyl, pyridyl, pyrimidinyl, pyridazinyl and pyrazinyl.

Embodiment 9. The compound of any one of the preceding Embodiments, wherein the structural unit

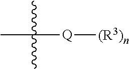

in Formula (I) satisfies Formula (Ib)

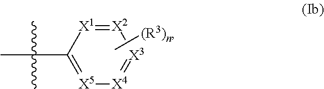

wherein

all of X¹, X², X³, X⁴ and X⁵ are CH; or

one or two of X¹, X², X³, X⁴ and X⁵ are N, and rest of them are CH.

Embodiment 10. The compound of Embodiment 9, wherein the structural unit

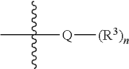

of Formula (I) satisfies Formula (Ic)

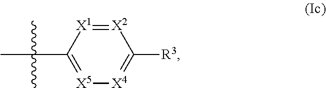

wherein

all of X¹, X², X⁴ and X⁵ are CH;

X² is N, and X¹, X⁴ and X⁵ are CH; or

X¹ is N, and X², X⁴ and X⁵ are CH.

Embodiment 11. The compound of Embodiment 9, wherein

both of X¹ and X² are N, and X⁴ and X⁵ are CH;

both of X² and X⁴ are N, and X¹ and X⁵ are CH;

both of X¹ and X⁴ are N, and X² and X⁵ are CH; or

both of X¹ and X⁵ are N, and X² and X⁴ are CH.

Embodiment 12. The compound of any one of the preceding Embodiments, wherein halo is F, Cl or Br.

Embodiment 13. The compound of any one of the preceding Embodiments, wherein n is 1 or 2.

Embodiment 14. The compound of any one of the preceding Embodiments, wherein R³ is independently selected from the group consisting of OCHF₂, C(CH₃)₂OH, cyclopropyl, CH₃, CF₃,

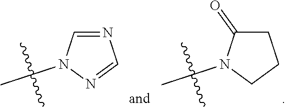 [SMILES: *n1cncn1 ; *N1CCCC1=O]

Embodiment 15. A compound, selected from the group consisting of the compounds in Table 1, Table 2 and Examples, or a stereoisomeric or a tautomeric form thereof:

or a pharmaceutically acceptable salt, N-oxide, or a solvate thereof.

Embodiment 16. A pharmaceutical composition, which comprises the compound of any one of Embodiments 1 to 15, and which further comprises at least one pharmaceutically acceptable excipient.

Embodiment 17. The compound of any one of Embodiments 1 to 15, or the pharmaceutical composition of Embodiment 16, for use as a medicament.

Embodiment 18. The compound of any one of Embodiments 1 to 15, or the pharmaceutical composition of Embodiment 16, for use in the prevention or treatment of an HBV infection or of an HBV-induced disease in a subject in need thereof.

Embodiment 19. The compound of any one of Embodiments 1 to 15, or the pharmaceutical composition of Embodiment 16, for use in the prevention or treatment of chronic hepatitis B.

Embodiment 20. A method of treating an HBV infection or an HBV-induced disease in an individual in need thereof, comprising administering to the individual a therapeutically effective amount of the compound of any one of Embodiments 1 to 15 or the pharmaceutical composition of Embodiment 16.

Embodiment 21. A product comprising a first compound and a second compound as a combined preparation for simultaneous, separate or sequential use in the prevention or treatment of an HBV infection or of an HBV-induced disease in a subject in need thereof, wherein said first compound is different from said second compound, wherein said first compound is the compound of any one of Embodiments 1 to 15 or the pharmaceutical composition of Embodiment 16, and wherein said second compound is another HBV inhibitor.

Embodiment 22. The product of Embodiment 21, wherein said second compound is another HBV inhibitor which is selected from the group consisting of: therapeutic agents selected from HBV combination drugs, HBV vaccines, HBV DNA polymerase inhibitors, immunomodulatory agents, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (CTLA-4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

Embodiment 23. A compound as defined in any one of Embodiments 1 to 15 or the pharmaceutical composition of Embodiment 16 for use in the prevention or treatment of an HBV infection or an HBV-induced disease in a subject, wherein the compound or pharmaceutical composition is administered to the subject in combination with another HBV inhibitor.

In an attempt to help the reader of the present application, the description has been separated in various paragraphs or sections. These separations should not be considered as disconnecting the substance of a paragraph or section from the substance of another paragraph or section. To the contrary, the present description encompasses all the combinations of the various sections, paragraphs and sentences that can be contemplated.

Each of the relevant disclosures of all references cited herein is specifically incorporated by reference. The following examples are offered by way of illustration, and not by way of limitation.

### EXAMPLES

Exemplary compounds useful in methods of the present disclosure will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent. Reactions may be heated employing conventional heating or microwave heating. Reactions may also be conducted in sealed pressure vessels above the normal reflux temperature of the solvent.

Compounds of Formula (I) may be converted to their corresponding salts using methods known to one of ordinary skill in the art. For example, an amine of Formula (I) is treated with trifluoroacetic acid, HCl, or citric acid in a solvent such as Et₂O, CH₂Cl₂, THF, MeOH, chloroform, or isopropanol to provide the corresponding salt form. Alternately, trifluoroacetic acid or formic acid salts are obtained as a result of reverse phase HPLC purification conditions. Crystalline forms of pharmaceutically acceptable salts of compounds of Formula (I), may be obtained in crystalline form by recrystallization from polar solvents (including mixtures of polar solvents and aqueous mixtures of polar solvents) or from non-polar solvents (including mixtures of non-polar solvents).

Where the compounds according to this present disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure.

Compounds represented as “stereomeric mixture” (means a mixture of two or more stereoisomers and includes enantiomers, diastereomers and combinations thereof) are separated by SFC resolution.

Compounds may be obtained as single forms, such as single enantiomers, by form-specific synthesis, or by resolution. Compounds may alternately be obtained as mixtures of various forms, such as racemic (1:1) or non-racemic (not 1:1) mixtures. Where racemic and non-racemic mixtures of enantiomers are obtained, single enantiomers may be isolated using conventional separation methods known to one of ordinary skill in the art, such as chiral chromatography, recrystallization, diastereomeric salt formation, derivatization into diastereomeric adducts, biotransformation, or enzymatic transformation. Where regioisomeric or diastereomeric mixtures are obtained, as applicable, single isomers may be separated using conventional methods such as chromatography or crystallization.

### 1. GENERAL INFORMATION

### Chemical Names

Chemical names were generated using the chemistry software: ACD/ChemSketch, and may follow preferably the IUPAC rules.

### General Procedure for LCMS Methods

The High Performance Liquid Chromatography (HPLC) measurement was performed using a LC pump, a diode-array (DAD) or a UV detector and a column as specified in the respective methods.

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW). Data acquisition was performed with appropriate software.

Compounds are described by their experimental retention times (Rt) and ions. If not specified differently, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, etc.). All results were obtained with experimental uncertainties that are commonly associated with the method used. Hereinafter, “SQD” means Single Quadrupole Detector, “MSD” Mass Selective Detector, “RT” room temperature, “BEH” bridged ethylsiloxane/silica hybrid, “DAD” Diode Array Detector, “HSS” High Strength silica, “Q-Tof” Quadrupole Time-of-flight mass spectrometers, “CLND”, ChemiLuminescent Nitrogen Detector, “ELSD” Evaporative Light Scanning Detector.

### NMR Analysis

¹H NMR spectra were recorded on a) a Bruker DPX 400 MHz spectrometer or b) a Bruker Avance 400 MHz spectrometer or c) Bruker Avance III 400 MHz spectrometer or d) Bruker Avance 600 MHz spectrometer or e) a Bruker Avance NEO 400 MHz spectrometer or f) Bruker model AVIII 400 spectrometer g) ZKNJ BIXI-1 300 MHz, Bruker Avance III 400 MHz or h) Bruker AVANCE Neo 400 MHz.

NMR spectra were recorded at ambient temperature unless otherwise stated. Data are reported as follow: chemical shift in parts per million (ppm) relative to TMS (δ=0 ppm) on the scale, integration, multiplicity (s=singlet, d=doublet, t=triplet, q=quartet, quin=quintet, sext=sextet, sept=septet, m=multiplet, b=broad, or a combination of these), coupling constant(s) J in Hertz (Hz).

### MS Analysis

Mass spectra were obtained on a Shimadzu LCMS-2020 MSD or Agilent 1200/G6110A MSD using electrospray ionization (ESI) in positive mode unless otherwise indicated.

### 2. ABBREVIATIONS

TABLE 3

| | |
|---|---|
| μw | Microwave |
| ADDP | 1,1′-(Azodicarbonyl)dipiperidine |
| AIBN | 2,2′-azobis(2-methylpropionitrile) |
| AlMe₃ | Trimethylaluminium |
| aq. | Aqueous |
| atm | Atmosphere |
| Boc₂O | Di-tert-butyl dicarbonate |
| BOC | tert-Butyloxycarbonyl |
| BODIPY | Boron-dipyrromethene |
| BPO | Benzoyl peroxide |
| br | broad |
| CA | Capsid assembly |
| CbzCl | Benzyl carbonochloride |
| DAST | (Diethylamino)sulfur trifluoride |
| DBU | 1,8-Diazabicyclo[5.4.0]undec-7-ene |
| DBAD | di-tert-butyl azodicarboxylate |
| DCE | 1,2-Dichloroethane |
| DCM | Dichloromethane |
| dd | Doublet of doublets |
| DEA | Diethylamine |
| DIPE | Diisopropyl ether |
| DIPEA/DIEA | N,N-diisopropylethylamine |
| DMAP | 4-dimethylaminopyridine |
| DMF | Dimethylformamide |
| DMF-DMA | Dimethylformamide-dimethylacetal |
| DMSO | Dimethylsulfoxide |
| DNA | Deoxyribonucleic Acid |
| EDCI | N-(3-Dimethylaminopropyl)-N′-ethylcarbodiimide hydrochloride |
| ESI | Electrospray ionization |
| Et₃N | Triethylamine |
| Et₂O | Ether, diethyl ether |
| EtOAc/EA | Ethyl acetate |
| EtOH | Ethanol |
| FCC | Normal-phase silica gel chromatography |
| g | grams |
| h/hr | hour |
| HATU | 1-[bis(dimethylamino)methylene]-1H-1,2,3-triazolo[4,5- |
| | b]pyridinium-3-oxide hexafluorophosphate |
| HBV | Hepatitis B virus |
| HOAc | Acetic acid |
| HOBT | 1H-benzo[d][1,2,3]triazol-1-ol |
| HPLC | High Performance Liquid Chromatography |
| Hz | Hertz |
| IPA | Isopropyl alcohol |
| i-PrNH₂ | Isopropylamine |
| i-PrOH/IPA | Isopropyl alcohol |
| KOtBu | Potassium tert-butoxide |
| LAH | Lithium aluminum hydride |
| LCMS | Liquid Chromatography Mass Spectrometry |
| LHMDS, LiHMDS | Lithium bis(trimethylsilyl)amide |
| M | molar |
| m | multiplet |
| MeCN/ACN | Acetonitrile |
| MeOH | Methanol |
| mg | milligrams |
| MHz | megahertz |
| min | Minutes |
| mL | Milliliters |
| μL | microliters |
| mmol | Millimole |
| Mn(dpm)₃ | Tris(((Z)-2,2,6,6-tetramethyl-5-oxohept-3-en-3-yl)oxy)manganese |
| μmol | micromole |
| MS | Mass spectra |
| MsCl | Mesityl chloride |
| m/z | Mass to charge ratio |
| N | normal |
| NaOAc/AcONa | Sodium acetate |
| NBS | N-bromsuccinimide |
| NMR | Nuclear Magnetic Resonance |
| o/n | Overnight |
| PCR | Polymerase chain reaction |
| PE | Petroleum ether |
| PMPA | 9-(2-Phosphonyl-methoxypropyl)adenine |
| ppm | Parts per million |
| ppt | precipitate |
| Py | pyridine |
| RNA | Ribonucleic Acid |
| Rₜ | Retention time |
| rt | Room temperature |
| S | singlet |
| sat. | Saturated |
| SEMCl | (2-(chloromethoxy)ethyl)trimethylsilane |
| SFC | Supercritical Fluid Chromatography |
| t | triplet |
| T₃P | Propanephosphonic acid anhydride |
| TBAF | tetrabutylammonium fluoride |
| TBAI | Tetrabutylammonium iodide |
| TBD | 1,5,7-Triazabicyclo[4.4.0]dec-5-ene |
| TBSCl | tert-butylchlorodimethylsilane |
| TBDPSCl | tert-butylchlorodiphenylsilane |
| TBDPS | tert-butyldiphenylsilyl |
| TEA | triethylamine |
| TEBAC | benzyltriethylammonium chloride |
| TFA | Trifluoroacetic acid |
| THF | Tetrahydrofuran |
| TLC | Thin Layer Chromatography |
| TLR | Toll-like receptor |
| TMSCl | Chlorotrimethylsilane |
| TNF | Tumor necrosis factor |
| TsCl | Tosyl chloride |
| V or volumes | Volume in milliliters of solvent per gram of substrate |
| Δ | Heating under reflux |
| Cbz | benzyloxycarbonyl |

### Experimental Procedures

### Intermediate A

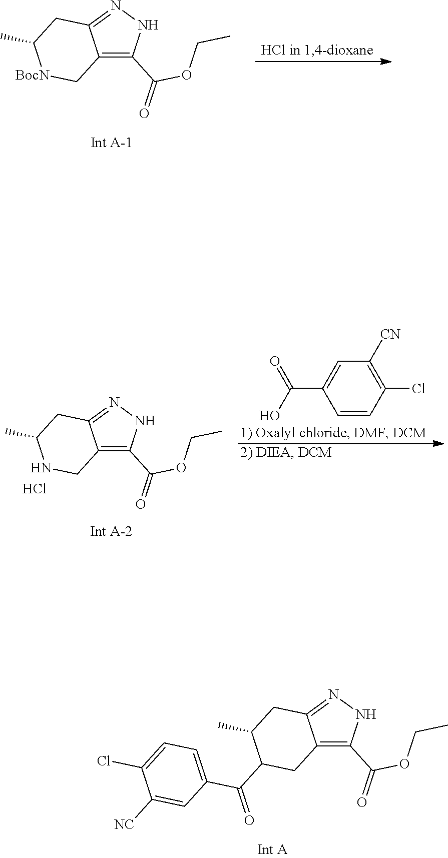

### Intermediate A-2

### (R)-Ethyl 6-methyl-4,5,6,7-tetrahydro-2H-pyrazolo[4,3-c]pyridine-3-carboxylate hydrochloride

A mixture of (R)-5-tert-butyl 3-ethyl 6-methyl-6,7-dihydro-2H-pyrazolo[4,3-c]pyridine-3,5(4H)-dicarboxylate Int A-1 (10.0 g, 100% purity, 32.3 mmol) in 4 M hydrochloride in 1,4-dioxane (100 mL) was stirred at room temperature for 2 hours under nitrogen atmosphere. The mixture was concentrated under reduced pressure to give the title compound (8.0 g, 95% purity from ¹H NMR, 96% yield) as yellow solids without further purification. LC-MS (ESI): R_{T}=0.5 min, mass calcd. for C₁₀H₁₅N₃O₂209.1, m/z found 210.2 [M+H]⁺.

### Intermediate A

### (R)-Ethyl 5-(4-chloro-3-cyanobenzoyl)-6-methyl-4,5,6,7-tetrahydro-2H-pyrazolo[4,3-c]pyridine-3-carboxylate

To a mixture of 4-chloro-3-cyanobenzoic acid (5.9 g, 100% purity, 32.5 mmol) in dichloromethane (60 mL) was added dropwise N,N-dimethylformamide (235 mg, 3.22 mmol) and oxalyl dichloride (4 mL, 47.3 mmol) at 0° C. After stirred at room temperature for 2 hours under nitrogen atmosphere, the mixture became clear until no gas escaped, then concentrated under reduced pressure. The resulting residue and (R)-ethyl 6-methyl-4,5,6,7-tetrahydro-2H-pyrazolo[4,3-c]pyridine-3-carboxylate hydrochloride Int A-2 (8.0 g, 95% purity, 30.9 mmol) was dissolved in dichloromethane (60 mL), N-ethyl-N-isopropylpropan-2-amine (16 mL, 96.8 mmol) was added dropwise at 0° C. After stirring for 1 hour, the mixture was poured into water (60 mL) and adjusted to pH 5˜6 with 1 M hydrochloride aqueous solution, extracted with dichloromethane (60 mL) twice. The combined organic layers were concentrated under reduced pressure. The resulting residue was purified by C18 column (acetonitrile:water (+0.02% ammonium acetate)=10% to 75%) to give the title compound (9.3 g, 90% purity, 69.1% yield) as yellow solids. LC-MS (ESI): R_{T}=1.38 min, mass calcd. for C₁₈H₁₇ClN₄O₃ 372.1, m/z found 373.1 [M+H]⁺. ¹H NMR (400 MHz, DMSO-d₆) 13.78-13.34 (m, 1H), 8.24-8.08 (m, 1H), 7.87-7.80 (m, 2H), 5.49-5.15 (m, 1H), 4.63-4.06 (m, 4H), 3.16 (br s, 1H), 2.72-2.54 (m, 1H), 1.41-1.04 (m, 6H).

### Intermediate B

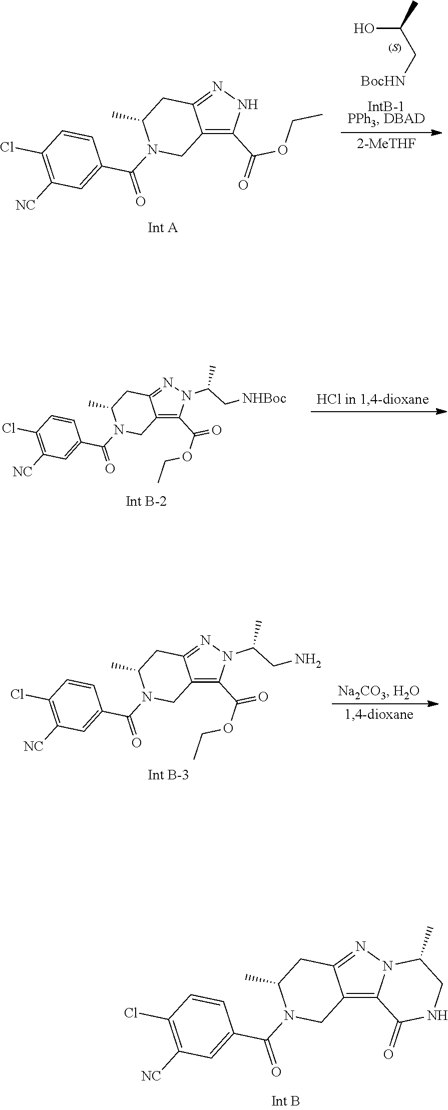

### Intermediate B-2

### (R)-Ethyl 2-((R)-1-((tert-butoxycarbonyl)amino)propan-2-yl)-5-(4-chloro-3-cyanobenzoyl)-6-methyl-4,5,6,7-tetrahydro-2H-pyrazolo[4,3-c]pyridine-3-carboxylate

To a solution of (R)-ethyl 5-(4-chloro-3-cyanobenzoyl)-6-methyl-4,5,6,7-tetrahydro-2H-pyrazolo[4,3-c]pyridine-3-carboxylate Int A (400 mg, 90% purity, 0.97 mmol), (S)-tert-butyl (2-hydroxypropyl)carbamate Int B-1 (260 mg, 1.48 mmol) and triphenylphosphine (500 mg, 1.91 mmol) in 2-methyltetrahydrofuran (5 mL) was added di-tert-butyl azodicarboxylate (470 mg, 2.04 mmol) at room temperature. After stirring at 40° C. for 10 hours, the reaction mixture was quenched with water (25 mL), extracted with ethyl acetate (50 mL) twice. The combined organic layers were washed with brine (30 mL), dried over Na₂SO₄₍ₛ₎ and filtered. The filtrate was concentrated and purified by C18 column (acetonitrile:water=5% to 100%) to give the crude. The crude was purified by silica gel column chromatography (petroleum ether:ethyl acetate=1:1) to give the title compound (465 mg, 98% purity from LCMS, 89.0% yield) as white solids. LC-MS (ESI): R_{T}=1.71 min, mass calcd. for C₂₆H₃₂ClN₅O₅ 529.2, m/z found 530.1 [M+H]⁺.

### Intermediate B-3

### (R)-Ethyl 2-((R)-1-aminopropan-2-yl)-5-(4-chloro-3-cyanobenzoyl)-6-methyl-4,5,6,7-tetrahydro-2H-pyrazolo[4,3-c]pyridine-3-carboxylate hydrochloride

A solution of (R)-ethyl 2-((R)-1-((tert-butoxycarbonyl)amino)propan-2-yl)-5-(4-chloro-3-cyanobenzoyl)-6-methyl-4,5,6,7-tetrahydro-2H-pyrazolo[4,3-c]pyridine-3-carboxylate Int B-2 (465 mg, 98% purity, 0.86 mmol) in 4 M hydrochloride in 1,4-dioxane (5 mL) was stirred at room temperature for 2 hours. The mixture was concentrated under reduced pressure to give the title compound (410 mg, 97% purity from LCMS, 99.2% yield) as yellow solids. LC-MS (ESI): RT=1.58 min, mass calcd. for C₂₁H₂₄ClN₅O₃ 429.2, m/z found 430.3 [M+H]⁺.

### Intermediate B

### 2-Chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile

To a solution of (R)-ethyl 2-((R)-1-aminopropan-2-yl)-5-(4-chloro-3-cyanobenzoyl)-6-methyl-4,5,6,7-tetrahydro-2H-pyrazolo[4,3-c]pyridine-3-carboxylate hydrochloride Int B-3 (410 mg, 97% purity, 0.85 mmol) in 1,4-dioxane (2 mL) was added saturated sodium carbonate aqueous solution (2 mL) at room temperature. After stirring at room temperature for 3 hours, the reaction mixture was quenched with water (10 mL), extracted with ethyl acetate (30 mL) twice. The combined organic layers were washed with brine (20 mL), dried over Na₂SO₄₍ₛ₎ and filtered. The filtrate was concentrated to give the title compound (355 mg, 91% purity from LCMS, 98.7% yield) as yellow solids. LC-MS (ESI): R_{T}=1.42 min, mass calcd. for C₁₉H₁₈ClN₅O₂ 383.1, m/z found 384.0 [M+H]⁺.

### Intermediate C

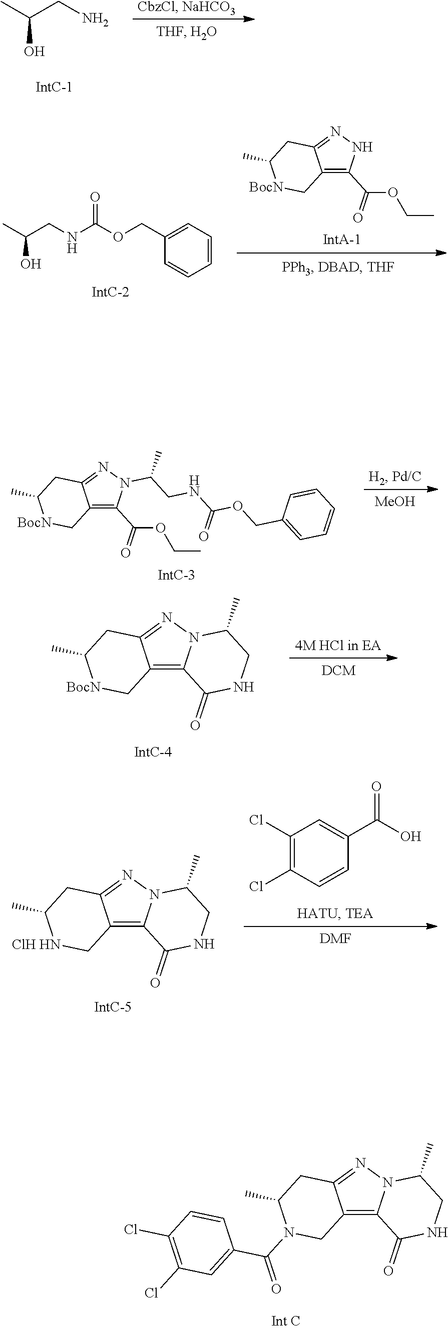

### Intermediate C-2

### (S)-Benzyl (2-hydroxypropyl)carbamate

To a mixture of (S)-(+)-1-Amino-2-propanol Int C-1 (10.0 g, 100% purity, 133.1 mmol) and sodium bicarbonate (33.0 g, 393 mmol) in tetrahydrofuran (100 mL) and water (50 mL) was added benzyl carbonochloride (34.0 g, 199.3 mmol) at 0° C. After stirring at room temperature for 16 hours, the mixture was poured into water (100 mL), extracted with ethyl acetate (150 mL) for three times. The combined organic layers were concentrated under reduced pressure, the resulting residue was purified by silica gel column chromatography (petroleum ether:ethyl acetate=100:1 to 1:1) to give the title compound (25 g, 95% purity from ¹H NMR, 85% yield) as a colorless oil. LC-MS (ESI): R_{T}=1.26 min, mass calcd. for C₁₁H₁₅NO₃ 209.1, m/z found 210.0 [M+H]⁺. ¹H NMR (400 MHz, DMSO-d₆) δ 7.43-7.29 (m, 5H), 7.21-7.11 (m, 1H), 5.07-4.98 (m, 2H), 4.67-4.59 (m, 1H), 3.68-3.56 (m, 1H), 3.02-2.84 (m, 2H), 1.08-0.95 (m, 3H).

### Intermediate C-3

### (R)-5-tert-Butyl 3-ethyl 2-((R)-1-(((benzyloxy)carbonyl)amino)propan-2-yl)-6-methyl-6,7-dihydro-2H-pyrazolo[4,3-c]pyridine-3,5(4H)-dicarboxylate

To a mixture of (S)-benzyl (2-hydroxypropyl)carbamate Int C-2 (5.0 g, 95% purity, 22.7 mmol) and (R)-5-tert-butyl 3-ethyl 6-methyl-6,7-dihydro-2H-pyrazolo[4,3-c]pyridine-3,5(4H)-dicarboxylate IntA-1 (8.8 g, 95% purity, 27.0 mmol) in tetrahydrofuran (75 mL) were added triphenylphosphine (9.5 g, 36.2 mmol) and di-tert-butyl azodicarboxylate (8.3 g, 36.0 mmol) at 0° C. under nitrogen atmosphere. After stirring at room temperature for 3 hours under nitrogen atmosphere, the mixture was concentrated and the resulting residue was purified by silica gel column chromatography (dichloromethane:methanol=100:1 to 20:1) to give the title compound (10.0 g, 100% purity from LCMS, 88% yield) as colorless oil. LC-MS (ESI): R_{T}=1.84 min, mass calcd. for C₂₆H₃₆N₄O₆ 500.3, m/z found 501.4 [M+H]⁺.

### Intermediate C-4

### (3R,7R)-tert-Butyl 3,7-dimethyl-10-oxo-3,4,7,8,9,10-hexahydropyrido[4′,3′:3,4]pyrazolo[1,5-a]pyrazine-2(1H)-carboxylate

To a mixture of (R)-5-tert-butyl 3-ethyl 2-((R)-1-(((benzyloxy)carbonyl)amino)propan-2-yl)-6-methyl-6,7-dihydro-2H-pyrazolo[4,3-c]pyridine-3,5(4H)-dicarboxylate Int C-3 (18.0 g, 100% purity, 36.0 mmol) in methanol (150 mL) was added palladium on carbon (2.88 g, 10% purity, 2.71 mmol) at room temperature. After stirring at 50° C. and 50 psi under hydrogen atmosphere for 16 hours, the mixture was filtered and the filtrate was concentrated under reduced pressure. The resulting residue was purified by silica gel column chromatography (dichloromethane:methanol=100:1 to 18:1) to give the title compound (12.0 g, 90% purity from ¹H NMR, 94% yield) as white solids. ¹H NMR (400 MHz, DMSO-d₆) δ 8.14 (s, 1H), 4.91 (d, J=16.8 Hz, 1H), 4.76-4.63 (m, 1H), 4.43-4.33 (m, 1H), 4.10-3.97 (m, 1H), 3.66-3.59 (m, 1H), 3.35-3.29 (m, 1H), 2.80 (dd, J=16.0 and 6.0 Hz, 1H), 2.55 (d, J=16.0 Hz, 1H), 1.44-1.42 (m, 12H), 1.02 (d, J=6.8 Hz, 3H).

### Intermediate C-5

### (3R,7R)-3,7-Dimethyl-1,2,3,4,8,9-hexahydropyrido[4′,3′:3,4]pyrazolo[1,5-a]pyrazin-10(7H)-one hydrochloride

To a solution of (3R,7R)-tert-butyl 3,7-dimethyl-10-oxo-3,4,7,8,9,10-hexahydropyrido[4′,3′:3,4]pyrazolo[1,5-a]pyrazine-2(1H)-carboxylate Int C-4 (15 g, 95% purity, 0.044 mol) in dichloromethane (100 mL) was added 4 M hydrochloride in ethyl acetate (100 mL, 0.4 mol) at 0° C. After stirring at room temperature for 2 hours, the mixture was concentrated under reduced pressure to give title compound (11 g, 100% purity from LCMS, 96% yield) as white solids. LC-MS (ESI): R_{T}=0.34 min, mass calcd. for C₁₁H₁₇ClN₄O 220.1, m/z found 221.1 [M+H]⁺.

### Intermediate C

### (3R,7R)-2-(3,4-Dichlorobenzoyl)-3,7-dimethyl-1,2,3,4,8,9-hexahydropyrido[4′,3′:3,4]pyrazolo[1,5-a]pyrazin-10(7H)-one

To a solution of 3,4-dichlorobenzoic acid (10 μg, 52.4 mmol), 1-[bis(dimethylamino)methylene]-1H-1,2,3-triazolo[4,5-b]pyridinium-3-oxide hexafluorophosphate (24 g, 65.5 mmol) in N,N-dimethylformamide (100 mL) was added (3R,7R)-3,7-dimethyl-1,2,3,4,8,9-hexahydropyrido[4′,3′:3,4]pyrazolo[1,5-a]pyrazin-10(7H)-one hydrochloride Int C-5 (11 g, 100% purity, 42.8 mmol) and triethylamine (40 mL, 287 mmol). After stirring at room temperature for 16 hours, the mixture was diluted with water (500 mL) and extracted with ethyl acetate (100 mL) twice. The combined organic layers were washed with brine (500 mL) twice, dried over Na₂SO₄₍ₛ₎ and filtered. The filtrate was concentrated under reduced pressure to give crude, which was purified by silica gel column chromatography (dichloromethane: ethyl acetate=10:1 to 3:1) to give the title compound (14.6 g, 100% purity from LCMS, 87% yield) as white solids. LC-MS (ESI): R_{T}=1.47 min, mass calcd. for C₁₈H₁₈Cl₂N₄O₂ 392.1, m/z found 393.1 [M+H]⁺.

### Intermediate D

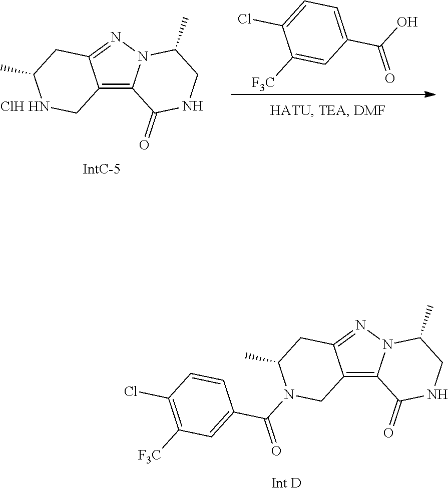

### Intermediate D

### (3R,7R)-2-(4-Chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one

To the solution of IntC-5 (11.0 g, 90% purity, 38.6 mmol) and 4-chloro-3-(trifluoromethyl)benzoic acid (13.0 g, 57.9 mmol) in N,N-dimethylformamide (200 mL) were added triethylamine (25 mL, 193 mmol) and 2-(3H-[1,2,3]triazolo[4,5-b]pyridin-3-yl)-1,1,3,3-tetramethylisouronium hexafluorophosphate (25.0 g, 65.8 mmol) at 0° C. After stirred at room temperature for 1 hour, the mixture was acidified to pH=6 with 0.5 M hydrochloride aqueous solution and extracted with ethyl acetate (100 mL) twice. The combined organic layers were washed with water (100 mL) for three times and brine (100 mL), dried over Na₂SO₄₍ₛ₎ and filtered. The filtrate was concentrated under reduced pressure to give a residue, which was purified by silica gel column chromatography (petroleum ether:ethyl acetate=2:1 to 1:3) to give the title compound (18.0 g, 90% purity from LCMS, 98% yield) as yellow solids. LC-MS (ESI): R_{T}=1.50 min, mass calcd. for C₁₉H₁₈ClF₃N₄O₂ 426.1, m/z found 427.0 [M+H]⁺.

### Intermediate E

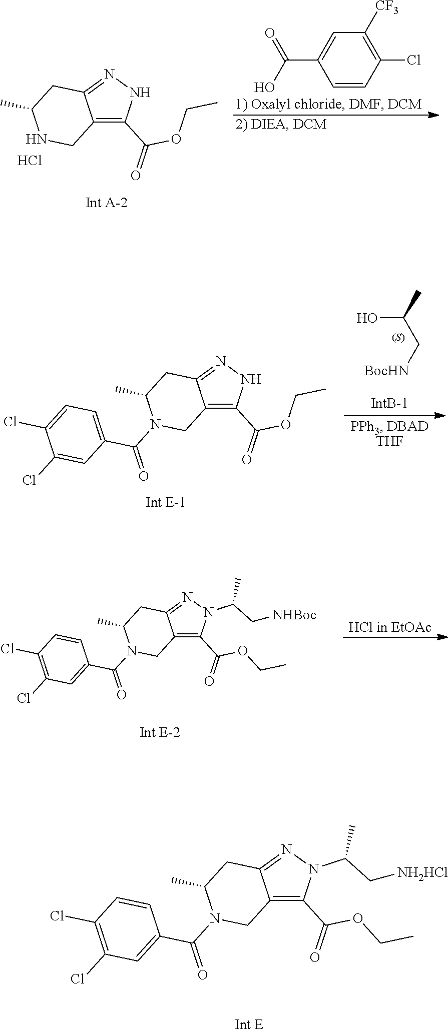

### Intermediate E-1

### Ethyl (R)-5-(3,4-dichlorobenzoyl)-6-methyl-4,5,6,7-tetrahydro-2H-pyrazolo[4,3-c]pyridine-3-carboxylate

To a solution of 3,4-dichlorobenzoic acid (5.62 g, 29.4 mmol) and N,N-dimethylformamide (5 drops, 3.35 mmol) in dichloromethane (200 mL) was added oxalyl chloride (3.73 mL, 44.1 mmol) dropwise at 0° C. under nitrogen atmosphere. Then the mixture was warmed to room temperature and stirred for 3 hours. After the reaction was completed, the mixture was concentrated under reduced pressure to give a crude product. To a solution of the crude mixture and (R)-ethyl 6-methyl-4,5,6,7-tetrahydro-2H-pyrazolo[4,3-c]pyridine-3-carboxylate hydrochloride IntA-2 (7.15 g, 29.1 mmol) in dichloromethane (200 mL) was added N-ethyl-N-isopropylpropan-2-amine (14.6 mL, 88.3 mmol) dropwise at 0° C., then the mixture was stirred at room temperature overnight. After the reaction was completed, the solvent was removed under reduced pressure to give a brown residue, which was purified by silica column chromatography (petroleum ether:ethyl acetate=1:1) to give the title compound (8.75 g, 98% purity, 76% yield) as white solids.

### Intermediate E-2

### Ethyl (R)-2-((R)-1-((tert-butoxycarbonyl)amino)propan-2-yl)-5-(3,4-dichlorobenzoyl)-6-methyl-4,5,6,7-tetrahydro-2H-pyrazolo[4,3-c]pyridine-3-carboxylate

To a mixture of ethyl (R)-2,5-bis(3,4-dichlorobenzoyl)-6-methyl-4,5,6,7-tetrahydro-2H-pyrazolo[4,3-c]pyridine-3-carboxylate IntE-1 (9.10 g, 90% purity, 21.4 mmol) and tert-butyl (S)-(2-hydroxypropyl)carbamate IntB-1 (5.63 g, 80% purity, 25.7 mmol) in tetrahydrofuran (100 mL) was added triphenylphosphine (11.2 g, 42.7 mmol) and (E)-di-tert-butyl diazene-1,2-dicarboxylate (12.3 g, 53.4 mmol) portions at 0° C. under nitrogen atmosphere. After stirred at room temperature overnight under nitrogen atmosphere, the mixture was concentrated and the resulting residue was purified by silica gel column chromatography (petroleum ether:Acetone=5:1 to 2:1) to give the desired product (11.8 g, 90% purity from LCMS, 92% yield) as white solids. LC-MS (ESI): R_{T}=1.79 min, mass calcd. for C₂₅H₃₂Cl₂N₄O₅ 538.2, m/z found 539.1 [M+H]⁺.

### Intermediate E

### Ethyl (R)-2-((R)-1-aminopropan-2-yl)-5-(3,4-dichlorobenzoyl)-6-methyl-4,5,6,7-tetrahydro-2H-pyrazolo[4,3-c]pyridine-3-carboxylate hydrogen chloride

To a solution of ethyl (R)-2-((R)-1-((tert-butoxycarbonyl)amino)propan-2-yl)-5-(3,4-dichlorobenzoyl)-6-methyl-4,5,6,7-tetrahydro-2H-pyrazolo[4,3-c]pyridine-3-carboxylate IntE-2 (11.8 g, 90% purity, 19.7 mmol) in ethyl acetate (50 mL) was added 4 M hydrogen chloride in ethyl acetate (50 mL) at 0° C. After stirred at room temperature for 1 hour, the mixture was concentrated to give the title compound (10.7 g, 90% purity from LCMS, 95% yield) as white solids. LC-MS (ESI): R_{T}=1.58 min, mass calcd. for C₂₀H₂₄Cl₂N₄O₃ 438.1, m/z found 439.0 [M+H]⁺.

### Intermediate F

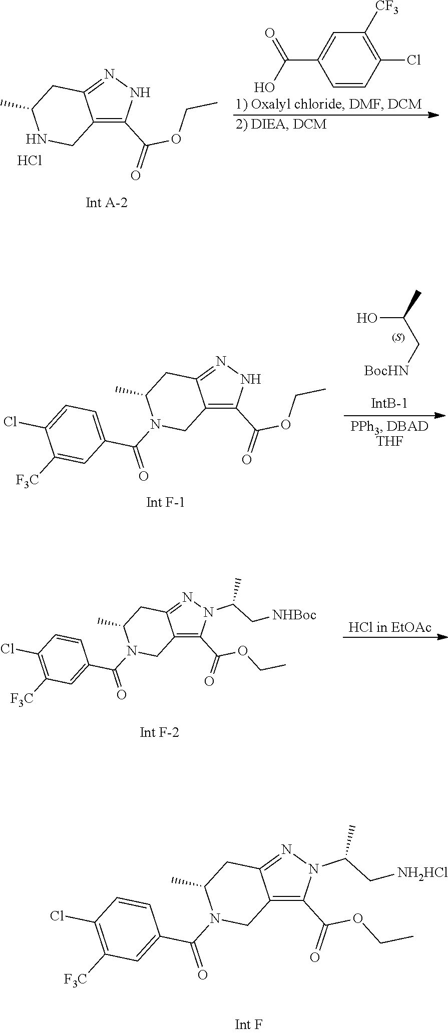

### Intermediate F-1

### Ethyl (R)-5-(4-chloro-3-(trifluoromethyl)benzoyl)-6-methyl-4,5,6,7-tetrahydro-2H-pyrazolo[4,3-c]pyridine-3-carboxylate

To a solution of 4-chloro-3-(trifluoromethyl)benzoic acid (11 g, 49.0 mmol) in dichloromethane (100 mL) was added oxalyl dichloride (9.2 g, 72.5 mmol) dropwise and N,N-dimethylformamide (460 mg, 6.29 mmol) at 0° C. After stirred at 25° C. for 1 hour, the mixture was concentrated and dissolved in dichloromethane (100 mL), then was added to a solution of (R)-ethyl 6-methyl-4,5,6,7-tetrahydro-2H-pyrazolo[4,3-c]pyridine-3-carboxylate hydrochloride IntA-2 (11.9 g, 100% purity, 48.4 mmol) in dichloromethane (100 mL). The resulted mixture was stirred at 0° C. for 15 minutes, followed by N-ethyl-N-isopropylpropan-2-amine (25 g, 193 mmol) was added dropwise at 0° C. The mixture was stirred at 25° C. for 3 hours, then was poured into water (200 mL), extracted with dichloromethane (200 mL) twice. The combined organic layers were concentrated. The residue was dissolved with ethanol (200 mL) and potassium carbonate (16 g, 116 mmol) was added. The mixture was stirred at 25° C. for 3 hours. The reaction mixture was poured into water (200 mL), extracted with dichloromethane (200 mL) twice. The combined organic layers were concentrated to give desired product (19 g, 100% purity from LCMS, 93% yield) as white solids. LC-MS (ESI): RT=1.50 min, mass calcd. for C₁₈H₁₇ClF₃N₃O₃ 415.1, m/z found 415.9. [M+H]⁺.

### Intermediate F-2

### Ethyl (R)-2-((R)-1-((tert-butoxycarbonyl)amino)propan-2-yl)-5-(4-chloro-3-(trifluoromethyl)benzoyl)-6-methyl-4,5,6,7-tetrahydro-2H-pyrazolo[4,3-c]pyridine-3-carboxylate

To a mixture of ethyl (R)-5-(4-chloro-3-(trifluoromethyl)benzoyl)-6-methyl-4,5,6,7-tetrahydro-2H-pyrazolo[4,3-c]pyridine-3-carboxylate IntF-1 (9.10 g, 95% purity, 20.8 mmol) and tert-butyl (S)-(2-hydroxypropyl)carbamate IntB-1 (5.47 g, 80% purity, 25.0 mmol) in tetrahydrofuran (100 mL) was added triphenylphosphine (10.9 g, 41.6 mmol) and di-tert-butyl azodicarboxylate (12.0 g, 52.1 mmol) portions at 0° C. under nitrogen atmosphere. After stirred at room temperature overnight under nitrogen atmosphere, the mixture was concentrated and the resulting residue was purified by silica gel column chromatography (petroleum ether:ethyl acetate=4:1 to 1:1) to give the desired product (11.5 g, 90% purity from LCMS, 87% yield) as white solids. LC-MS (ESI): R_{T}=1.86 min, mass calcd. for C₂₆H₃₂ClF₃N₄O₅ 572.2, m/z found 573.0 [M+H]⁺.

### Intermediate F

### Ethyl (R)-2-((R)-1-aminopropan-2-yl)-5-(4-chloro-3-(trifluoromethyl)benzoyl)-6-methyl-4,5,6,7-tetrahydro-2H-pyrazolo[4,3-c]pyridine-3-carboxylate hydrochloride

To a solution of ethyl (R)-2-((R)-1-((tert-butoxycarbonyl)amino)propan-2-yl)-5-(4-chloro-3-(trifluoromethyl)benzoyl)-6-methyl-4,5,6,7-tetrahydro-2H-pyrazolo[4,3-c]pyridine-3-carboxylate IntF-3 (23.0 g, 90% purity, 36.1 mmol) in ethyl acetate (100 mL) was added 4 M hydrogen chloride in ethyl acetate (100 mL) at 0° C. After stirred at room temperature for 1 hour, the mixture was concentrated to give the title compound (20.0 g, 90% purity from LCMS, 98% yield) as white solids. LC-MS (ESI): R_{T}=1.70 min, mass calcd. for C₂₁H₂₄ClF₃N₄O₃ 472.1, m/z found 473.3 [M+H]⁺.

### Compounds 1A, 1B, 1C and 1D

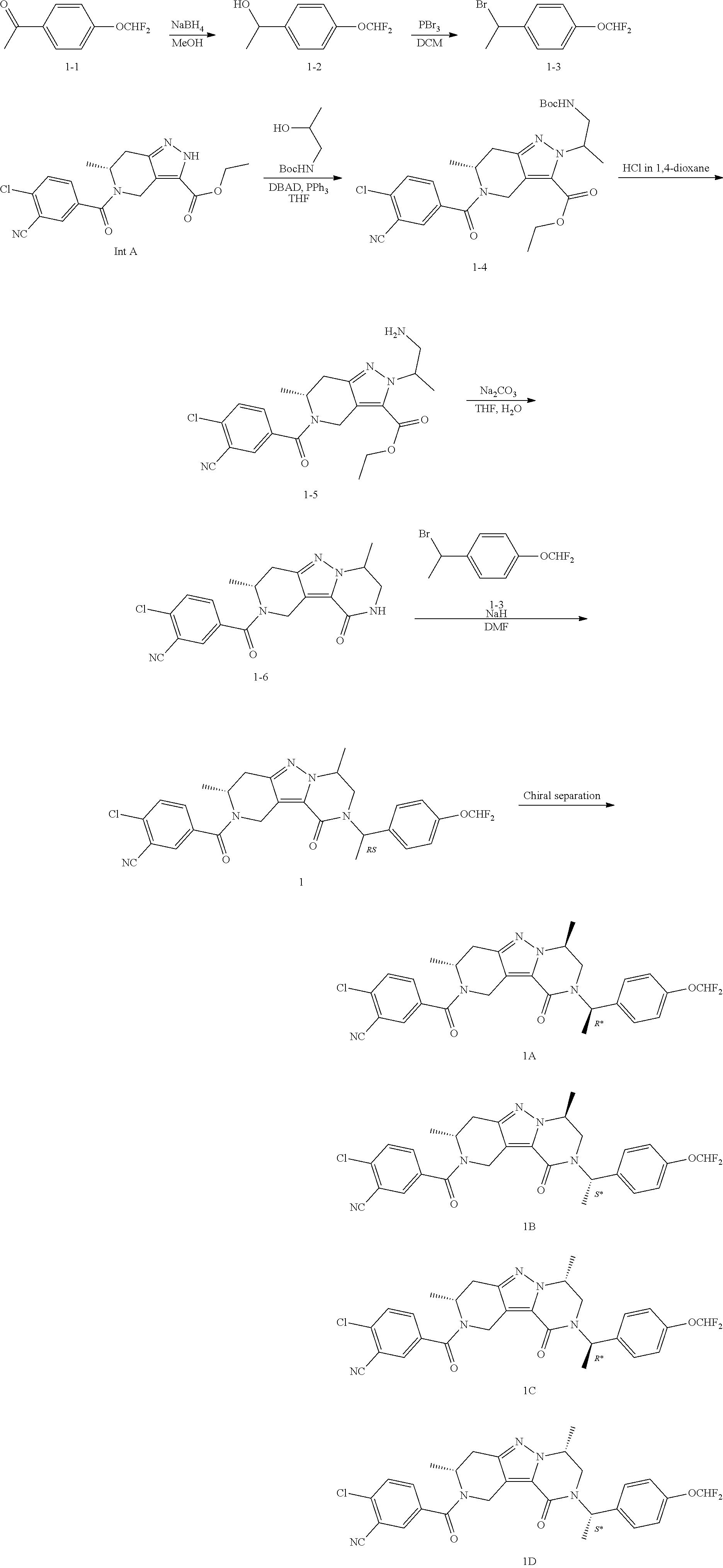

### Intermediate 1-2

### 1-(4-(Difluoromethoxy)phenyl)ethanol

To a solution of 1-(4-(difluoromethoxy)phenyl)ethanone 1-1 (5.0 g, 26.9 mmol) in methanol (50 mL) was slowly added sodium borohydride (3.0 g, 79.3 mmol) at 0° C. After stirring at room temperature for three hours, the reaction was quenched with acetone (50 mL) dropwise and concentrated to give a residue, which was dissolved into ethyl acetate (50 mL) and washed with brine (50 mL) twice, dried over Na₂SO₄₍ₛ₎ and filtered. The filtrate was concentrated to give the title compound (5.3 g, 90% purity from ¹H NMR, 94% yield) as colorless oil. ¹H NMR (400 MHz, CDCl₃) 7.37 (d, J=8.4 Hz, 2H), 7.10 (d, J=8.4 Hz, 2H), 6.49 (t, J=73.6 Hz, 1H), 4.90 (q, J=6.4 Hz, 1H), 1.84 (br s, 1H), 1.48 (d, J=6.4 Hz, 3H).

### Intermediate 1-3

### 1-(1-Bromoethyl)-4-(difluoromethoxy)benzene

To a solution of 1-(4-(difluoromethoxy)phenyl)ethanol 1-2 (500 mg, 90% purity, 2.39 mmol) in dichloromethane (5 mL) was slowly added phosphorus(III) bromide (650 mg, 2.40 mmol) at 0° C. After stirred at room temperature for 2 hours, the reaction mixture was poured into ice water (20 mL) and extracted with dichloromethane (30 mL) twice. The combined organic layers were washed with saturated sodium bicarbonate solution (50 mL) twice and brine (50 mL) twice, dried over Na₂SO₄₍ₛ₎ and filtered. The filtrate was concentrated under reduced pressure to give the title compound (590 mg, 95% purity from ¹H NMR, 93% yield) as colorless oil. ¹H NMR (400 MHz, CDCl₃) δ 7.44 (d, J=8.4 Hz, 2H), 7.09 (d, J=8.8 Hz, 2H), 6.51 (t, J=73.6 Hz, 1H), 5.20 (q, J=7.2 Hz, 1H), 2.03 (d, J=6.8 Hz, 3H).

### Intermediate 1-4

### (6R)-Ethyl 2-(1-((tert-butoxycarbonyl)amino)propan-2-yl)-5-(4-chloro-3-cyanobenzoyl)-6-methyl-4,5,6,7-tetrahydro-2H-pyrazolo[4,3-c]pyridine-3-carboxylate

To a solution of (R)-ethyl 5-(4-chloro-3-cyanobenzoyl)-6-methyl-4,5,6,7-tetrahydro-2H-pyrazolo[4,3-c]pyridine-3-carboxylate Int A (900 mg, 95% purity, 2.29 mmol) and tert-butyl (2-hydroxypropyl)carbamate (1.6 g, 9.13 mmol) in tetrahydrofuran (10 mL) was added triphenylphosphine (1.1 g, 4.19 mmol) at 0° C., followed by the addition of (E)-di-tert-butyl diazene-1,2-dicarboxylate (940 mg, 4.08 mmol). After stirring at 50° C. for 2 hours, the mixture was concentrated and diluted with water (20 mL) extracted with ethyl acetate (20 mL) twice. The combined organic layers were washed with brine (25 mL), dried over Na₂SO₄₍ₛ₎ and filtered.

The filtrate was concentrated and purified by C18 column (acetonitrile:water=55% to 60%) to give the title compound (1.1 g, 94% purity, 85% yield) as white solids. LC-MS (ESI): R_{T}=1.70 min, mass calcd. for C₂₆H₃₂ClN₅O₅ 529.2, m/z found 530.0 [M+H]⁺.

### Intermediate 1-5

### (6R)-Ethyl 2-(1-aminopropan-2-yl)-5-(4-chloro-3-cyanobenzoyl)-6-methyl-4,5,6,7-tetrahydro-2H-pyrazolo[4,3-c]pyridine-3-carboxylate

The solution of (6R)-ethyl 2-(1-((tert-butoxycarbonyl)amino)propan-2-yl)-5-(4-chloro-3-cyanobenzoyl)-6-methyl-4,5,6,7-tetrahydro-2H-pyrazolo[4,3-c]pyridine-3-carboxylate 1-4 (1.1 g, 94% purity, 0.195 mmol) in 4 M hydrochloride in 1,4-dioxane (15 mL) was stirred at room temperature for 2 hours. The mixture was concentrated under reduced pressure to give the title compound (900 mg, 100% purity from LCMS, 98.9% yield) as white solids. LC-MS (ESI): RT=1.53 min, mass calcd. for C₂₁H₂₄ClN₅O₃ 429.2, m/z found 430.1 [M+H]⁺.

### Intermediate 1-6

### 2-Chloro-5-((3R)-3,7-dimethyl-10-oxo-1,2,3,4,7,8,9,10-octahydropyrido[4′,3′:3,4]pyrazolo[1,5-a]pyrazine-2-carbonyl)benzonitrile

To a solution of (6R)-ethyl 2-(1-aminopropan-2-yl)-5-(4-chloro-3-cyanobenzoyl)-6-methyl-4,5,6,7-tetrahydro-2H-pyrazolo[4,3-c]pyridine-3-carboxylate hydrochloride 1-5 (900 mg, 100% purity, 1.93 mmol) in tetrahydrofuran (10 mL) and water (10 mL) was added sodium carbonate (1.3 g, 12.3 mmol) at 0° C. After stirring at 0° C. for 3 hours, the mixture was poured into water (30 mL) and extracted with ethyl acetate (30 mL) twice. The combined organic layers were washed with brine (30 mL), dried over Na₂SO₄₍ₛ₎, filtrated. The filtrate was concentrated and purified by C18 column (acetonitrile:water=55% to 60%) to give the title compound (700 mg, 100% purity, 94.5% yield) as white solids. LC-MS (ESI): R_{T}=1.41 min, mass calcd. for C₁₉H₁₈ClN₅O₂ 383.1, m/z found 384.0 [M+H]⁺.

### Compound 1

### 2-chloro-5-((3R)-9-(1-(4-(difluoromethoxy)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile

To a solution of 2-chloro-5-((3R)-3,7-dimethyl-10-oxo-1,2,3,4,7,8,9,10-octahydropyrido[4′,3′:3,4]pyrazolo[1,5-a]pyrazine-2-carbonyl)benzonitrile 1-6 (700 mg, 100% purity, 1.73 mmol) in dry N,N-dimethylformamide (10 mL) was added 60% sodium hydride in mineral oil (200 mg, 60% purity, 5 mmol) slowly at 0° C. After stirring at 0° C. for 20 minutes, the 5-(bromomethyl)benzo[c][1,2,5]oxadiazole 1-3 (1.3 g, 5.18 mmol) was added dropwise and the mixture was stirred at 0° C. for 2 hours. The reaction mixture was quenched with water (10 mL) and extracted with ethyl acetate (10 mL) twice. The combined organic layers were washed with brine (15 mL), dried over Na₂SO₄₍ₛ₎ and filtered. The filtrate was concentrated and purified by C18 column (acetonitrile:water=50% to 60%) to give the title compound (800 mg, 100% purity, 79.2% yield) as yellow oil. LC-MS (ESI): R_{T}=1.67 min, mass calcd. for C₂₈H₂₆ClF₂N₅O₃ 553.2, m/z found 553.9 [M+H]⁺. ¹H NMR (400 MHz, CDCl₃) δ 7.75 (s, 1H), 7.63-7.57 (m, 2H), 7.39-7.32 (m, 2H), 7.11 (d, J=8.0 Hz, 2H), 6.51 (t, J=73.6 Hz, 1H), 6.11-5.23 (m, 2H), 4.88-4.14 (m, 3H), 3.32-3.21 (m, 2H), 3.15-2.94 (m, 1H), 2.76-2.62 (m, 1H), 1.65-1.58 (m, 3H), 1.53 (d, J=6.4 Hz, 3H), 1.35-1.22 (m, 3H). ¹⁹F NMR (376 MHz, CDCl₃) −81.13.

### Compounds 1A, 1B, 1C and 1D

### 2-Chloro-5-((3R,7S*)-9-((R*)-1-(4-(difluoromethoxy)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile (1A)

### 2-Chloro-5-((3R,7S*)-9-((S*)-1-(4-(difluoromethoxy)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile (1B)

### 2-Chloro-5-((3R,7R*)-9-((R*)-1-(4-(difluoromethoxy)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile (1C) and

### 2-Chloro-5-((3R,7R*)-9-((S*)-1-(4-(difluoromethoxy)phenyl)ethyl)-3,7-dimethyl-O-oxo-1,2,3,4,7,8,9,10-octahydropyrido[4′,3′:3,4]pyrazolo[1,5-a]pyrazine-2-carbonyl)benzonitrile (1D)

The racemate of 2-chloro-5-((3R)-9-(1-(4-(difluoromethoxy)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile 1 (850 mg, 100% purity, 1.44 mmol) was separated by chiral Prep. HPLC (separation condition: Column: Chiralpak IG 5 m 30*250 mm; Mobile Phase: MeOH:DCM=80:20 at 30 g/min; Col. Temp: 30° C.; Wavelength: 254 nm) to give the crude Fragment I (600 mg, 94% purity, 70.5% yield) as white solids and the title compound 1D (103.2 mg, 98.3% purity, 12.3% yield, 99.9% stereopure) as white solids. Fragment I (600 mg, 94% purity, 1.02 mmol) was separated by chiral Prep. HPLC (separation condition: Column: Chiralpak IB m 30*250 mm; Mobile Phase: Hex:EtOH=75:25 at 30 g/min; Col. Temp: 30° C.; Wavelength: 254 nm) to give the title compound 1A (68.6 mg, 99.7% purity, 12.2% yield, 100% stereopure) as white solids and the crude Fragment II (300 mg, 100% purity, 53.2% yield) as white solids. Fragment 11 (300 mg, 100% purity, 0.542 mmol) was separated by chiral Prep. SFC (Column: Chiralpak IA 5 m 4.6*250 mm; Mobile Phase: CO₂:EtOH=60:40 at 60 g/min; Col. Temp: 40° C.; Wavelength: 214 nm, Back pressure: 100 bar) to give the title compounds 1B (66.0 mg, 99.8% purity, 22.0% yield, 100% stereopure) as white solids and 1C (82.2 mg, 99.2% purity, 27.2% yield, 100% stereopure) as white solids.

### 1A:

LC-MS (ESI): R_{T}=3.528 min, mass calcd. for C₂₈H₂₆ClF₂N₅O₃ 553.2, m/z found 554.2 [M+H]⁺.

Chiral analysis (Column: Chiralpak IB 5 m 4.6*250 mm; Mobile Phase: Hex:EtOH=75:25 at 1 mL/min; Temp: 30° C.; Wavelength: 254 nm, Back, R_{T}=8.569 min). ¹H NMR (400 MHz, CDCl₃) δ 7.75 (s, 1H), 7.63-7.57 (m, 2H), 7.41-7.30 (m, 2H), 7.12 (d, J=8.4 Hz, 2H), 6.51 (t, J=73.2 Hz, 1H), 6.14-5.32 (m, 2H), 4.81-4.35 (m, 3H), 3.59-3.55 (m, 1H), 3.12-2.96 (m, 2H), 2.74-2.57 (m, 1H), 1.66-1.58 (m, 3H), 1.28 (s, 6H). ¹⁹F NMR (376 MHz, CDCl₃) δ −81.17.

### 1B:

LC-MS (ESI): R_{T}=4.304 min, mass calcd. for C₂₈H₂₆ClF₂N₅O₃ 553.2, m/z found 554.2 [M+H]⁺. Chiral analysis (Column: Chiralpak IA 5 m 4.6*250 mm; Mobile Phase: CO₂:EtOH=60:40 at 3.0 g/min; Col. Temp: 40° C.; Wavelength: 214 nm, Back pressure: 100 bar, Back, R_{T}=7.95 min). ¹H NMR (400 MHz, CDCl₃) δ 7.74 (s, 1H), 7.62-7.53 (m, 2H), 7.40-7.29 (m, 2H), 7.12 (d, J=8.4 Hz, 2H), 6.52 (t, J=73.6 Hz, 1H), 6.11-5.32 (m, 2H), 4.78-4.20 (m, 3H), 3.35-3.23 (m, 2H), 3.17-2.96 (m, 1H), 2.75-2.60 (m, 1H), 1.62-1.57 (m, 3H), 1.53 (d, J=6.4 Hz, 3H), 1.30-1.26 (m, 3H). ¹⁹F NMR (376 MHz, CDCl₃) δ −81.11.

### 1C:

LC-MS (ESI): R_{T}=4.206 min, mass calcd. for C₂₈H₂₆ClF₂N₅O₃ 553.2, m/z found 554.2 [M+H]⁺. Chiral analysis (Column: Chiralpak IA 5 m 4.6*250 mm; Mobile Phase: CO₂:EtOH=60:40 at 3.0 g/min; Col. Temp: 40° C.; Wavelength: 214 nm, Back pressure: 100 bar, Back, R_{T}=11.07 min). ¹H NMR (400 MHz, CDCl₃) δ 7.74 (s, 1H), 7.64-7.54 (m, 2H), 7.43-7.30 (m, 2H), 7.13 (d, J=8.4 Hz, 2H), 6.51 (t, J=73.6 Hz, 1H), 6.10-5.31 (m, 2H), 4.87-4.29 (m, 3H), 3.70-3.54 (m, 1H), 3.14-3.00 (m, 2H), 2.75-2.60 (m, 1H), 1.66-1.56 (m, 3H), 1.36-1.20 (m, 6H). ¹⁹F NMR (376 MHz, CDCl₃) δ −81.15.

### 1D:

LC-MS (ESI): R_{T}=3.282 min, mass calcd. for C₂₈H₂₆ClF₂N₅O₃ 553.2, m/z found 554.2 [M+H]⁺. Chiral analysis (Column: Chiralpak IG 5 m 4.6*250 mm; Mobile Phase: MeOH:DCM=80:20 at 1 mL/min; Temp: 30° C.; Wavelength: 254 nm, Back, R_{T}=9.076 min). ¹H NMR (400 MHz, CDCl₃) δ 7.75 (s, 1H), 7.63-7.57 (m, 2H), 7.39-7.32 (m, 2H), 7.11 (d, J=8.0 Hz, 2H), 6.51 (t, J=73.6 Hz, 1H), 6.11-5.23 (m, 2H), 4.88-4.14 (m, 3H), 3.32-3.21 (m, 2H), 3.15-2.94 (m, 1H), 2.76-2.62 (m, 1H), 1.65-1.58 (m, 3H), 1.53 (d, J=6.4 Hz, 3H), 1.35-1.22 (m, 3H). ¹⁹F NMR (376 MHz, CDCl₃) δ −81.1

### Compounds 2A and 2B

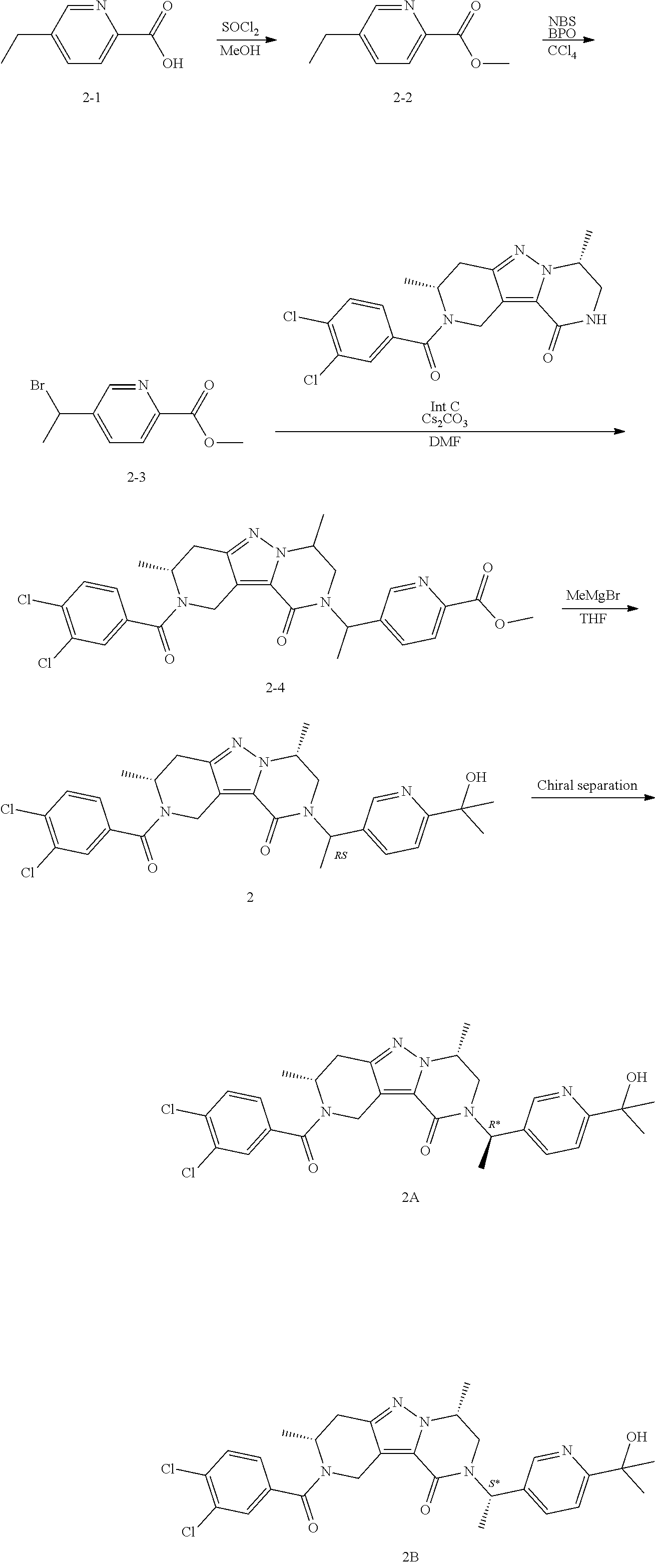

### Intermediate 2-2

### Methyl 5-ethylpicolinate

To a solution of 5-ethylpicolinic acid 2-1 (500 mg, 100% purity, 3.31 mmol) in methanol (10 mL) was added sulfurous dichloride (2.0 mL) at 0° C. After stirred at 70° C. for 16 hours under nitrogen atmosphere, the reaction was cooled down to room temperature. Water (1 mL) was added and the resulting mixture was concentrated under reduced pressure and then purified by purified C18 (acetonitrile:water (+0.02% ammonium acetate)=5% to 50%) to give the title compound (330 mg, 100% purity, 60% yield) as colorless oil. LC-MS (ESI): R_{T}=1.34 min, mass calcd. for C₉H₁₁NO₂ 165.1, m/z found 166.1 [M+H]⁺.

### Intermediate 2-3

### Methyl 5-(1-bromoethyl)picolinate

To a solution of methyl 5-ethylpicolinate 2-2 (310 mg, 100% purity, 1.88 mmol) in carbon tetrachloride (10 mL) at room temperature was added N-bromosuccinimide (350 mg, 1.97 mmol) followed by benzoyl peroxide (113 mg, 75% purity, 0.350 mmol) at room temperature. After stirring at 75° C. for 3 hours under nitrogen atmosphere, the reaction mixture was allowed to cool to room temperature and water (20 mL) was added followed by extraction with dichloromethane (3*20 mL). The combined organic layers were concentrated under reduced pressure. The residue was purified by C18 (acetonitrile:water (+0.02% ammonium acetate)=5% to 60%) to give the title product (390 mg, 100% purity from LCMS, 85% yield) as a colorless oil. LC-MS (ESI): R_{T}=1.42 min, mass calcd. for C₉H₁₀BrNO₂ 243.0, m/z found 244.0[M+H]⁺.

### Intermediate 2-4

### Methyl 5-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)picolinate

To a mixture of methyl 5-(1-bromoethyl)picolinate 2-3 (425 mg, 100% purity, 1.74 mmol) and (3R,7R)-2-(3,4-dichlorobenzoyl)-3,7-dimethyl-1,2,3,4,8,9-hexahydropyrido[4′,3′: 3,4]pyrazolo[1,5-a]pyrazin-10(7H)-one Int C (630 mg, 95% purity, 1.52 mmol) in N, N-dimethylformamide (10 mL) was added cesium carbonate (250 mg, 0.767 mmol) at room temperature. After stirring at 40° C. for 10 hours under nitrogen atmosphere, water (15 mL) was added. The resulting mixture was extracted with ethyl acetate (10 mL) for three times. The combined organic layers were concentrated under reduced pressure, the resulting residue was purified by C18 (acetonitrile:water (+0.02% ammonium acetate)=5% to 65%) to give the title product (700 mg, 100% purity from LCMS, 83% yield) as white solids. LC-MS (ESI): R_{T}=1.50 min, mass calcd. for C₂₇H₂₇Cl₂N₅O₄ 555.1, m/z found 556.3 [M+H]⁺.

### Compound 2

### (3R,7R)-2-(3,4-dichlorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of methyl 5-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)picolinate 2-4 (610 mg, 98.2% purity, 0.74 mmol) in tetrahydrofuran (1 mL) was added methylmagnesium bromide (1.0 M in tetrahydrofuran, 0.8 mL, 0.8 mmol) at 0° C. under nitrogen atmosphere. The resulting mixture was warm to 20° C. and stirred for 13 hours. The reaction mixture was quenched with saturated ammonium chloride solution (10 mL), extracted with acetate (10 mL) for three times, dried over Na₂SO₄₍ₛ₎, filtered. The filtrate was concentrated to get a residue, which was purified by prep. TLC (ethyl acetate) to give the title product (40 mg, 100% purity from LCMS, 40% yield) as white solids. LC-MS (ESI): R_{T}=1.45 min, mass calcd. for C₂₈H₃₁Cl₂N₅O₃ 555.2, m/z found 556.3 [M+H]⁺.

### Compounds 2A and 2B

### (3R,7R)-2-(3,4-dichlorobenzoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (2A) and (3R,7R)-2-(3,4-dichlorobenzoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (2B)

The racemate of (3R,7R)-2-(3,4-dichlorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one 2 (160 mg, 87% purity, 0.744 mmol) was separated by chiral prep. HPLC (separation method: Column: Chiralpak IE, 5 m 30*250 mm; Mobile Phase: MeOH:EtOH:DEA=50:50:0.2 at 60 mL/min; Col. Temp: 30° C.; Wavelength: 254 nm, Back pressure: 100 bar) to give the title compounds 2A (47.5 mg, 98.9% purity, 34% yield, 100% stereopure) as white solids and 2B (39.0 mg, 99.5% purity, 28% yield, 99.9% stereopure) as white solids.

### 2A:

LC-MS (ESI): R_{T}=4.021 min, mass calcd. for C₂₈H₃₁Cl₂N₅O₃ 555.2, m/z found 556.2 [M+H]⁺. Chiral analysis (Column: Superchiral IE, 5 m 4.6*250 mm; Mobile Phase: MeOH:EtOH:DEA=50:50:0.2 at 1 mL/min; Temp: 30° C.; Wavelength: 254 nm, Back pressure: 100 bar; R_{T}=8.395 min). ¹H NMR (400 MHz, CDCl₃) δ 8.55 (s, 1H), 7.75-7.67 (m, 1H), 7.54-7.50 (m, 2H), 7.39 (d, J=8.4 Hz, 1H), 7.28-7.25 (m, 1H), 6.11 (br s, 1H), 5.74-5.33 (m, 1H), 4.88-4.31 (m, 4H), 3.64 (d, J=12.8 Hz, 1H), 3.04-3.00 (m, 2H), 2.66 (d, J=16.4 Hz, 1H), 1.64-1.58 (m, 9H), 1.35-1.22 (m, 6H).

### 2B:

LC-MS (ESI): R_{T}=4.056 min, mass calcd. for C₂₈H₃₁Cl₂N₅O₃ 555.2, m/z found 556.2 [M+H]⁺. Chiral analysis (Column: Superchiral IE, 5 m 4.6*250 mm; Mobile Phase: MeOH:EtOH:DEA=50:50:0.2 at 1 mL/min; Temp: 30° C.; Wavelength: 254 nm, Back pressure: 100 bar; R_{T}=8.395 min). ¹H NMR (400 MHz, CDCl₃) δ 8.51 (s, 1H), 7.72-7.62 (m, 1H), 7.54-7.50 (m, 2H), 7.37 (d, J=8.4 Hz, 1H), 7.28-7.24 (m, 1H), 6.08 (br s, 1H), 5.82-5.28 (m, 1H), 4.94-4.21 (m, 4H), 3.36-3.25 (m, 2H), 3.17-2.92 (m, 1H), 2.74-2.62 (m, 1H), 1.64 (d, J=6.8 Hz, 3H), 1.58-1.55 (m, 9H), 1.33-1.21 (m, 3H).

### Compounds 3A and 3B

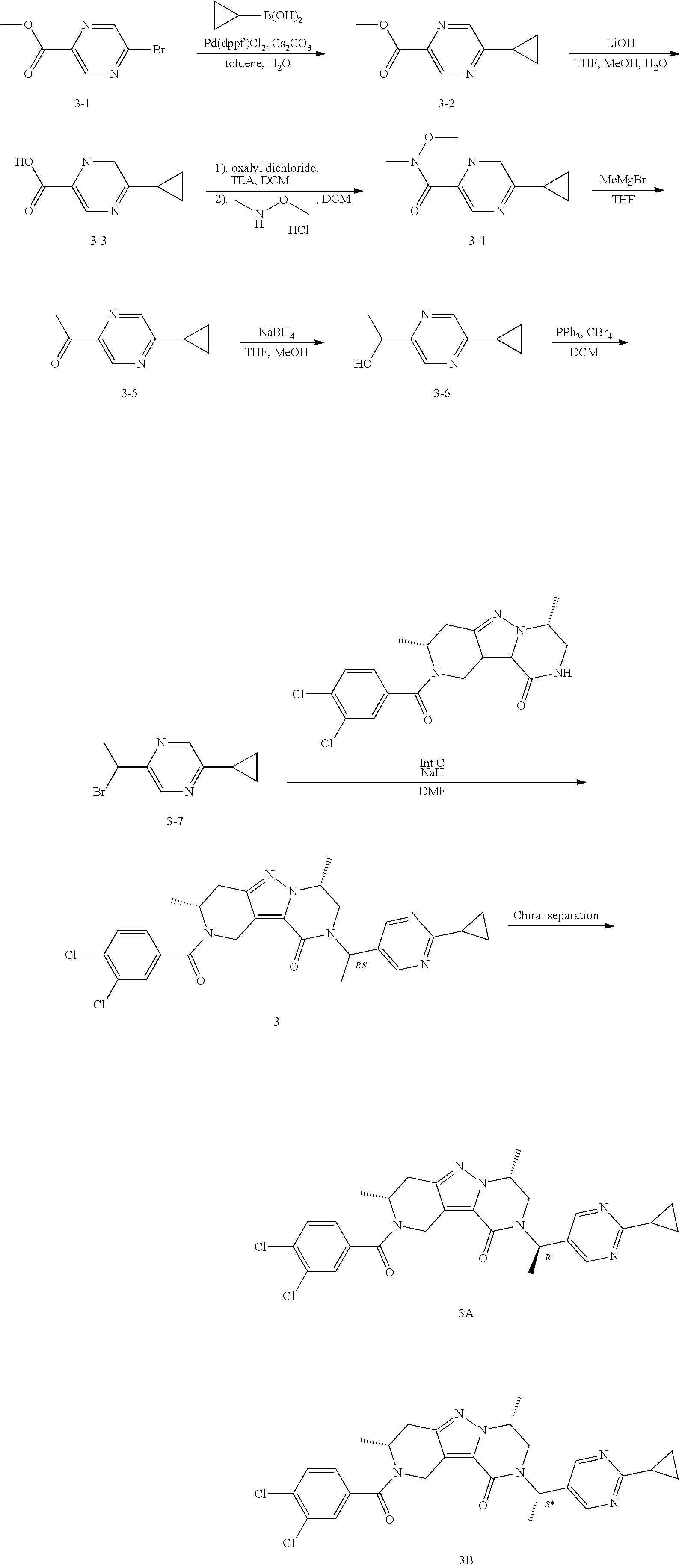

### Intermediate 3-2

### Methyl 5-cyclopropylpyrazine-2-carboxylate

A solution of methyl 5-bromopyrazine-2-carboxylate 3-1 (5.00 μg, 23.1 mmol), cyclopropylboronic acid (5 μg, 58.2 mmol), [1,1′-Bis(diphenylphosphino)ferrocene]dichloropalladium(II) (2.00 g, 2.73 mmol) and cesium carbonate (20 g, 61.4 mmol) in toluene (100 mL) and water (5 mL) was stirred at 100° C. under nitrogen atmosphere for 4 hours. The mixture was cooled down to room temperature and diluted with water (100 mL) and extracted with ethyl acetate (200 mL) twice. The combined organic layers were washed with brine (100 mL), dried over Na₂SO₄₍ₛ₎ and filtered. The resulting mixture was concentrated to give the residue, which was purified by silica gel chromatography column (ethyl acetate: petroleum ether=1: 6-1:3) to obtain title compound (3.50 g, 90% purity from ¹H NMR, 77% yield) as yellow solids. LC-MS (ESI): R_{T}=1.185 min, mass calcd. for C₉H₁₀N₂02, 178.1, m/z found 179.0 [M+H]⁺. ¹H NMR (400 MHz, DMSO-d₆) δ 9.00 (d, J=1.2 Hz, 1H), 8.77 (d, J=1.6 Hz, 1H), 3.90 (s, 3H), 2.38-2.31 (m, 1H), 1.19-1.15 (m, 2H), 1.08-1.04 (m, 2H).

### Intermediate 3-3

### 5-Cyclopropylpyrazine-2-carboxylic acid

To a mixture of methyl 5-cyclopropylpyrazine-2-carboxylate 3-2 (3.80 g, 90% purity, 19.2 mmol) in tetrahydrofuran (10 mL), methanol (5 mL) and water (5 mL) was added lithium hydroxide hydrate (950 mg, 22.639 mmol) at 0° C. After addition, the mixture was stirred at 25° C. for 2 hours. Then water (10 mL) was added, adjusted the pH with 1 N hydrogen chloride solution to 4-5. The mixture was extracted with dichloromethane (50 mL) for three times, dried over Na₂SO₄₍ₛ₎ and filtered. The filtrate was concentrated to give title compound (3.00 g, 98% purity from LCMS, 93% yield) as white solids. LC-MS (ESI): R_{T}=0.457 min, mass calcd. for C₈H₈N₂O₂, 164.1, m/z found 165.1 [M+H]⁺.

### Intermediate 3-4

### 5-Cyclopropyl-N-methoxy-N-methylpyrazine-2-carboxamide

To a mixture of 5-cyclopropylpyrazine-2-carboxylic acid 3-3 (3.00 g, 98% purity, 17.9 mmol) in dichloromethane (30 mL) was added oxalyl dichloride (4.6 g, 36.2 mmol) at 0° C. After addition, the mixture was stirred at 0° C. for 1 hour, then the reaction mixture was concentrated to give the residue. To a mixture of N, O-dimethylhydroxylamine hydrochloride (3.5 g, 35.9 mmol) in dichloromethane (40 mL) was added the residue (in dichloromethane 20 mL) at 0° C. for 30 minutes, the reaction mixture was quenched with water (30 mL) and extracted with dichloromethane (100 mL) twice. The combined organic layers were washed with brine (100 mL) and dried over Na₂SO₄₍ₛ₎ and filtered. The filtrate was concentrated to give the compound, which was purified by C18 column (acetonitrile:water (0.1% ammonium bicarbonate)=5% to 80%) to give the title compound (3.40 g, 95% purity from ¹H NMR, 87% yield) as yellow oil. LC-MS (ESI): R_{T}=1.306 min, mass calcd. for C₁₀H₁₃N₃O₂, 207.1, m/z found 208.1 [M+H]⁺.

### Intermediate 3-5

### 1-(5-Cyclopropylpyrazin-2-yl)ethanone

To a solution of 5-cyclopropyl-N-methoxy-N-methylpyrazine-2-carboxamide 3-4 (3.4 g, 95% purity, 15.59 mmol) in tetrahydrofuran (30 mL) was added methyl magnesium bromide (34 mL, 34 mmol) at 0° C. After stirring at 0° C. for 2 hours, the reaction mixture was quenched with saturated ammonium chloride aqueous solution (100 mL) and extracted with ethyl acetate (100 mL) twice. The combined organic layers were washed with brine (50 mL) twice, dried over Na₂SO₄₍ₛ₎ and filtered. The filtrate was concentrated under reduced pressure to give the title compound (2.4 g, 95% purity from ¹H NMR, 90% yield) as white solids. LC-MS (ESI): R_{T}=1.223 min, mass calcd. for C₉H₁₀N₂0, 162.1, m/z found 163.1 [M+H]⁺. ¹H NMR (400 MHz, DMSO-d₆) δ 8.92 (d, J=1.2 Hz, 1H), 8.75 (d, J=1.6 Hz, 1H), 2.61 (s, 3H), 2.38-2.32 (m, 1H), 1.18-1.15 (m, 2H), 1.08-1.04 (m, 2H).

### Intermediate 3-6

### 1-(5-Cyclopropylpyrazin-2-yl)ethanol

To a solution of 1-(5-cyclopropylpyrazin-2-yl)ethanone 3-5 (1.40 g, 95% purity, 3.14 mmol) in tetrahydrofuran (15 mL) and methanol (5 mL) was slowly added sodium borohydride (500 mg, 13.2 mmol) at 0° C. After stirring at room temperature for 2 hours, the reaction was quenched with acetone (2 mL) dropwise and concentrated to give the title compound (1.20 g, 95% purity from LCMS, 85% yield) as yellow oil. LC-MS (ESI): R_{T}=1.227 min, mass calcd. for C₉H₁₂N₂O, 164.1, m/z found 165.1 [M+H]⁺.

### Intermediate 3-7

### 2-(1-Bromoethyl)-5-cyclopropylpyrazine

To a solution of 1-(5-cyclopropylpyrazin-2-yl)ethanol 3-6 (1.00 g, 95% purity, 5.79 mmol) in dichloromethane (20 mL) was slowly added perbromomethane (2.90 g, 8.75 mmol) and triphenylphosphine (3.10 g, 11.8 mmol) at 0° C. After stirring at room temperature for 2 hours, the reaction mixture was poured into ice water (20 mL) and extracted with dichloromethane (20 mL) twice. The combined organic layers were washed with saturated sodium bicarbonate solution (10 mL) twice and brine (10 mL) twice, dried over Na₂SO₄₍ₛ₎ and filtered. The filtrate was concentrated under reduced pressure to get a residue, which was purified by silica gel column chromatography (petroleum ether:ethyl acetate=45:1) to give the title compound (1.20 g, 95% purity from LCMS, 87% yield) as colorless oil. LC-MS (ESI): R_{T}=1.594 min, mass calcd. for C₉H₁₁BrN₂, 226.0, m/z found 228.9. ¹H NMR (400 MHz, CDCl₃) δ 8.49 (d, J=1.6 Hz, 1H), 8.44 (d, J=1.6 Hz, 1H), 5.23 (q, J=7.2 Hz, 1H), 2.11-2.05 (m, 4H), 1.09-1.07 (m, 4H).

### Compound 3

### (3R,7R)-9-(1-(5-Cyclopropylpyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (200 mg, 100% purity, 0.51 mmol) in N,N-dimethylformamide (4 mL) was added sodium hydride (43 mg, 60% purity, 1.08 mmol) at 0° C. and stirred at 0° C. for 10 minutes. Then 2-(1-bromoethyl)-5-cyclopropylpyrazine 3-7 (140 mg, 95% purity, 0.59 mmol) was added and stirred at 25° C. for 2 hours. The mixture was added into water (5 mL) and extracted with dichloromethane (20 mL) twice. The combined organic layers were washed with water (10 mL), brine (10 mL), dried over Na₂SO₄₍ₛ₎ and filtered. The filtrate was concentrated under reduced pressure to get a residue, which was purified by C18 column (acetonitrile:water=5% to 80%) to give the title compound (200 mg, 95% purity from ¹H NMR, 69% yield) as white solids. LC-MS (ESI): R_{T}=1.508 min, mass calcd. for C₂₇H₂₈Cl₂N₆O₂, 538.2, m/z found 539.0 [M+H]⁺. ¹H NMR (400 MHz, DMSO-d₆) δ 8.58-8.50 (m, 2H), 7.75-7.73 (m, 2H), 7.44 (d, J=8.4 Hz, 1H), 5.87-5.76 (m, 1H), 5.45-5.21 (m, 1H), 4.52-4.34 (m, 2H), 4.17-3.85 (m, 2H), 3.58 (br s, 1H), 2.95-2.91 (m, 1H), 2.66-2.55 (m, 1H), 2.19 (br s, 1H), 1.57 (br s, 3H), 1.45-1.26 (m, 3H), 1.11 (br s, 3H), 1.04-0.93 (m, 4H).

### Compounds 3A and 3B

### (3R,7R)-9-((R*)-1-(5-cyclopropylpyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (3A) and (3R,7R)-9-((S*)-1-(5-cyclopropylpyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (3B)

A racemate of (3R,7R)-9-(1-(5-cyclopropylpyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 3 (300 mg, 95% purity, 0.53 mmol) was separated by chiral Prep. HPLC separation condition: (Column: Chiralpak IF 5 m 30*250 mm; Mobile Phase: ACN:IPA=70:30 at 25 mL/min; Temp: 30° C.; Wavelength: 254 nm) to give the crude chiral isomers, which were purified by C18 column (acetonitrile:water (0.1% ammonium bicarbonate)=5% to 80%) to give the title compounds 3A (110 mg, 99.4% purity, 38% yield, 100% stereopure) and 3B (110 mg, 99.8% purity, 38% yield, 99.93% stereopure) as white solids.

### 3A:

LC-MS (ESI): R_{T}=3.448 min, mass calcd. for C₂₇H₂₈Cl₂N₆O₂, 538.2, m/z found 539.3 [M+H]⁺.

Chiral analysis (Column: Chiralpak IF 5 m 4.6*250 mm; Mobile Phase: ACN:IPA=70:30 at 1 mL/min; Temp: 30° C.; Wavelength: 254 nm, R_{T}=6.246 min). ¹H NMR (400 MHz, DMSO-d₆) δ 8.58-8.50 (m, 2H), 7.75-7.73 (m, 2H), 7.45-7.43 (m, 1H), 5.86-5.75 (m, 1H), 5.47-5.23 (m, 1H), 4.45-4.12 (m, 3H), 3.85 (br s, 1H), 3.32-3.21 (m, 1H), 2.95-2.90 (m, 1H), 2.64-2.52 (m, 1H), 2.24-2.16 (m, 1H), 1.58 (br s, 3H), 1.26 (d, J=6.4 Hz, 3H), 1.17-1.11 (m, 3H), 1.04-1.02 (m, 2H), 0.93 (br s, 2H).

### 3B:

LC-MS (ESI): R_{T}=3.435 min, mass calcd. for C₂₇H₂₈Cl₂N₆O₂, 538.2, m/z found 539.3 [M+H]⁺.

Chiral analysis (Column: Chiralpak IF 5 m 4.6*250 mm; Mobile Phase: ACN:IPA=70:30 at 1 mL/min; Temp: 30° C.; Wavelength: 254 nm, R_{T}=8.322 min). ¹H NMR (400 MHz, DMSO-d₆) δ 8.57-8.49 (m, 2H), 7.74-7.72 (m, 2H), 7.45-7.42 (m, 1H), 5.88-5.76 (m, 1H), 5.44-5.23 (m, 1H), 4.53-4.16 (m, 3H), 3.60-3.54 (m, 2H), 2.95-2.91 (m, 1H), 2.65-2.54 (m, 1H), 2.19 (br s, 1H), 1.57 (br s, 3H), 1.44 (d, J=6.0 Hz, 3H), 1.13 (br s, 3H), 1.04-1.02 (m, 2H), 0.93 (br s, 2H).

### Compounds 4A and 4B

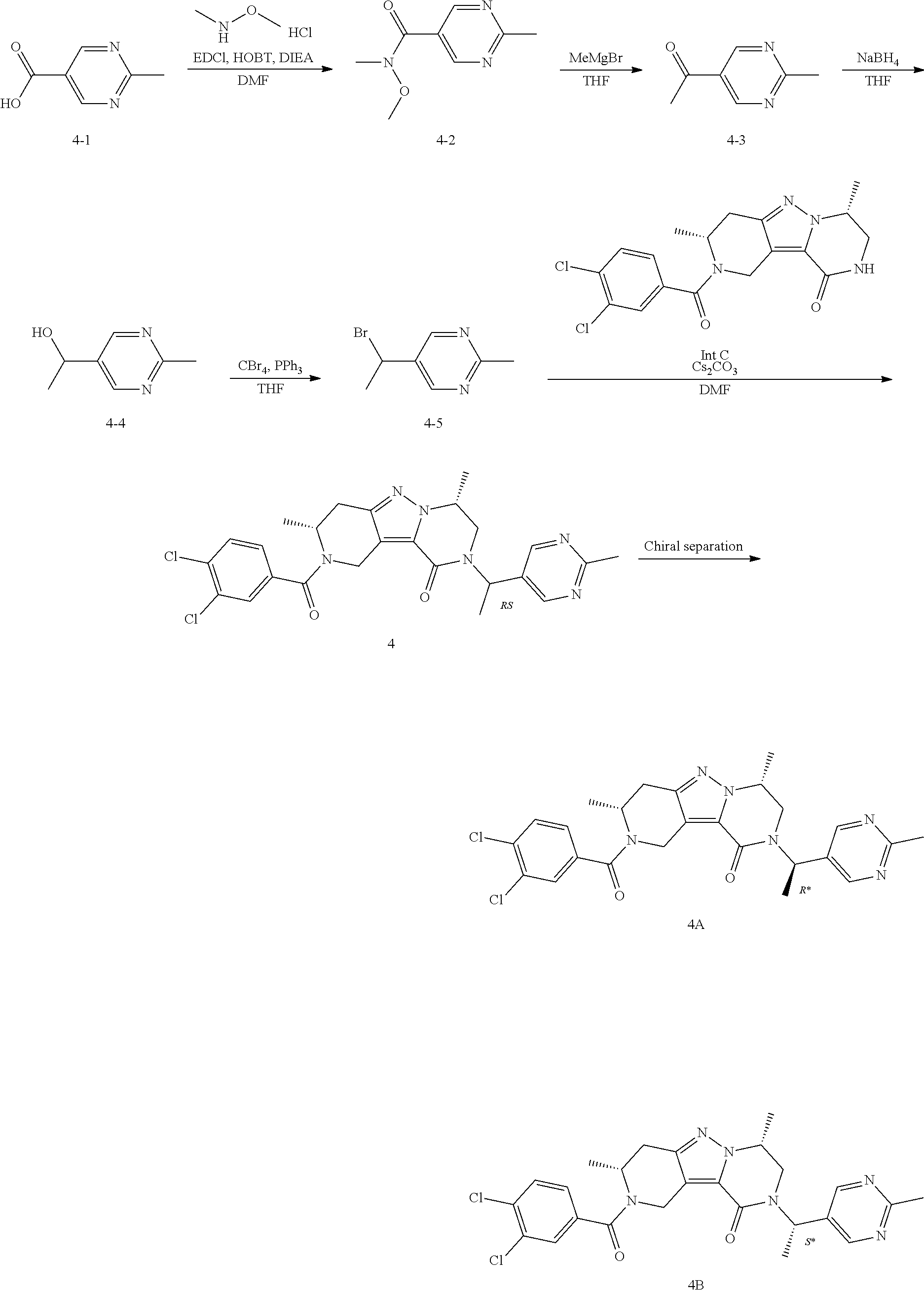

### Intermediate 4-2

### N-methoxy-N,2-Dimethylpyrimidine-5-carboxamide

To the solution of 2-Methylpyrimidine-5-carboxylic acid 4-1 (7.00 g, 45.6 mmol), N, O-dimethylhydroxylamine hydrochloride (10.2 g, 105 mmol), 1H-benzo[d][1,2,3]triazol-1-ol (10.1 g, 74.7 mmol) and N-ethyl-N-isopropylpropan-2-amine (20.2 g, 156 mmol) in dichloromethane (70 mL) was added N-(3-Dimethylaminopropyl)-N′-ethylcarbodiimide hydrochloride (20.1 g, 105 mmol) at room temperature under nitrogen atmosphere. After stirring at room temperature overnight, the mixture was diluted with water (150 mL) and extracted with ethyl acetate (150 mL) for three times. The combined organic layers were washed with brine (300 mL), dried over Na₂SO₄₍ₛ₎ and filtered. The filtrate was concentrated to give the residue, which was purified by silica gel column chromatography (petroleum ether:ethyl acetate=10:1) to give the title compound (7 g, 90% purity from ¹H NMR, 76% yield) as light yellow oil. ¹H NMR (300 MHz, CDCl₃) δ 8.98 (s, 2H), 3.56 (s, 3H), 3.37 (s, 3H), 2.76 (s, 3H).

### Intermediate 4-3

### 1-(2-Methylpyrimidin-5-yl)ethanone

To a solution of N-methoxy-N,2-dimethylpyrimidine-5-carboxamide 4-2 (6.7 g, 90% purity, 33.3 mmol) in tetrahydrofuran (70 mL) was added 1 M methylmagnesium bromide in tetrahydrofuran (55 mL, 55 mmol) at 0° C. After stirring at 0° C. for 1 hour, the reaction mixture was poured into ammonium chloride aqueous (100 mL) and extracted with ethyl acetate (150 mL) twice. The combined organic layers were washed with brine (200 mL), dried over Na₂SO₄₍ₛ₎ and filtered. The filtrate was concentrated to give the residue, which was purified by silica gel column chromatography (petroleum ether:ethyl acetate=10:1) to give the title compound (4.5 g, 90% purity from ¹H NMR, 89% yield) as yellow oil. ¹H NMR (300 MHz, CDCl₃) δ 9.12 (s, 2H), 2.81 (s, 3H), 2.62 (s, 3H).

### Intermediate 4-4

### 1-(2-Methylpyrimidin-5-yl) ethanol

To the solution of 1-(2-Methylpyrimidin-5-yl) ethanone 4-3 (2.00 g, 90% purity, 13.2 mmol) in tetrahydrofuran (25 mL) was added sodium borohydride (0.5 g, 13.2 mmol) at 0° C. After stirring at 0° C. for 1 hour, the mixture was quenched with saturated ammonium chloride aqueous solution (50 mL) and extracted with ethyl acetate (50 mL). The organic layer was washed with water (25 mL) twice, dried over Na₂SO₄₍ₛ₎ and concentrated under vacuum to give the residue, which was purified by C18 (acetonitrile:water=40% to 65%) to give the title product (200 mg, 90% purity from ¹H NMR, 9.8% yield) as yellow oil. ¹H NMR (300 MHz, CDCl₃) δ 8.63 (s, 2H), 4.97-4.89 (m, 1H), 4.22-4.06 (m, 1H), 2.71 (s, 3H), 1.55-1.53 (m, 3H).

### Intermediate 4-5

### 5-(1-Bromoethyl)-2-methylpyrimidine

To a solution of 1-(2-Methylpyrimidin-5-yl) ethanol 4-4 (200 mg, 90% purity, 1.30 mmol) in Tetrahydrofuran (6 mL) were added triphenylphosphine (700 mg, 2.11 mmol) and perbromomethane (700 mg, 2.67 mmol) at 0° C. After stirring at 25° C. for 0.5 hour, the mixture was filtered. The filtrate was concentrated under reduced pressure to give the residue, which was purified by silica gel column chromatography (petroleum ether:ethyl acetate=100:1) to give the title compound (180 mg, 90% purity from ¹H NMR, 61% yield) as red oil. ¹H NMR (300 MHz, CDCl₃) δ 8.75 (s, 2H), 5.17 (q, J=7.2 Hz, 1H), 2.79 (s, 3H), 2.12 (d, J=7.2 Hz, 3H).

### Compound 4

### (3R,7R)-2-(3,4-dichlorobenzoyl)-3,7-dimethyl-9-(1-(2-methylpyrimidin-5-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (180 mg, 100% purity, 0.458 mmol) in N,N-dimethylformamide (5 mL) were added cesium carbonate (0.313 g, 0.959 mmol) and 5-(1-bromoethyl)-2-methylpyrimidine 4-5 (0.169 g, 0.759 mmol) at 0° C. After stirring at 25° C. for 12 hours, the mixture was filtered. The filtrate was purified by C18 (acetonitrile:water=40% to 65%) to give the title compound (150 mg, 96% purity from LCMS, 61% yield) as yellow solids. LC-MS (ESI): R_{T}=1.58 min, mass calcd. for C₂₅H₂₆Cl₂N₆O₂ 512.2, m/z found 512.9 [M+H]⁺.

### Compounds 4A and 4B

### (3R,7R)-2-(3,4-dichlorobenzoyl)-3,7-dimethyl-9-((R*)-1-(2-methylpyrimidin-5-yl)ethyl)-1,2,3,4,8,9-hexahydropyrido[4′,3′:3,4]pyrazolo[1,5-a]pyrazin-10(7H)-one (4A) and (3R,7R)-2-(3,4-dichlorobenzoyl)-3,7-dimethyl-9-((S*)-1-(2-methylpyrimidin-5-yl)ethyl)-1,2,3,4,8,9-hexahydropyrido[4′,3′:3,4]pyrazolo[1,5-a]pyrazin-10(7H)-one (4B)

A racemate of (3R,7R)-2-(3,4-dichlorobenzoyl)-3,7-dimethyl-9-(1-(2-methylpyrimidin-5-yl)ethyl)-1,2,3,4,8,9-hexahydropyrido[4′,3′:3,4]pyrazolo[1,5-a]pyrazin-10(7H)-one 4 (150 mg, 96% purity, 0.280 mmol) was separated by chiral Prep. HPLC (separation condition: Column: Chiralpak IF 5 μm 20*250 mm; Mobile Phase: ACN-IPA=70:30 at 18 mL min; Temp: 30° C.; Wavelength: 254 nm) to afford 4A (75.2 mg, 98% purity from LCMS, 52% yield, 100% stereopure) as white solids and 4B (64.7 mg, 99% purity from LCMS, 44% yield, 99% stereopure) as off-white solids.

### 4A:

LC-MS (ESI): R_{T}=2.962 min, mass calcd. for C₂₅H₂₆Cl₂N₆O₂ 512.2, m/z found 513.2 [M+H]⁺. Chiral analysis (Column: Chiralpak IF 5 m 4.6*250 mm; Mobile Phase: ACN:IPA=70:30 at 1 mL/min; Temp: 30° C.; Wavelength: 254 nm; R_{T}=6.407 min). ¹H NMR (400 MHz, CDCl₃) δ 8.71 (s, 2H), 7.75-7.73 (m, 2H), 7.44 (dd, J=8.0, 2.0 Hz, 1H), 5.82-5.68 (m, 1H), 5.47-5.21 (m, 1H), 4.47-4.06 (m, 3H), 3.85-3.82 (m, 1H), 3.22-3.17 (m, 1H), 2.95-2.91 (m, 1H), 2.68-2.52 (m, 4H), 1.67-1.53 (m, 3H), 1.29 (d, J=6.4 Hz, 3H), 1.18-1.12 (m, 3H).

### 4B:

LC-MS (ESI): R_{T}=2.901 min, mass calcd. for C₂₅H₂₆Cl₂N₆O₂ 512.2, m/z found 513.2 [M+H]⁺. Chiral analysis (Column: Chiralpak IF 5 m 4.6*250 mm; Mobile Phase: ACN:IPA=70:30 at 1 mL/min; Temp: 30° C.; Wavelength: 254 nm; R_{T}=9.562 min). ¹H NMR (400 MHz, CDCl₃) δ 8.69 (s, 2H), 7.75-7.73 (m, 2H), 7.44 (dd, J=8.0, 1.6 Hz, 1H), 5.87-5.68 (m, 1H), 5.49-5.17 (m, 1H), 4.58-4.07 (m, 3H), 3.57-3.43 (m, 2H), 2.94-2.91 (m, 1H), 2.67-2.51 (m, 4H), 1.68-1.50 (m, 3H), 1.44 (d, J=6.4 Hz, 3H), 1.23-1.13 (m, 3H).

### Compounds 5A and 5B

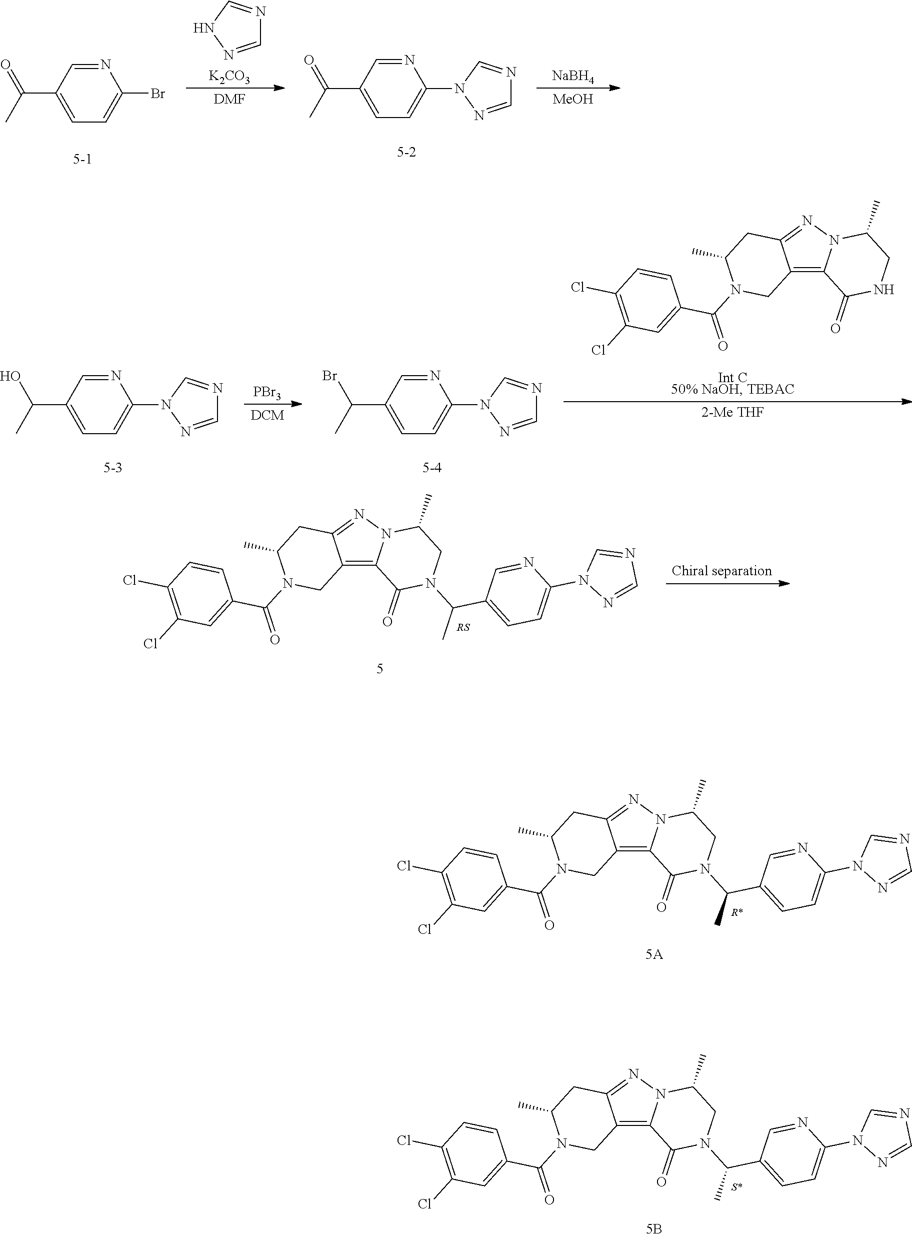

### Intermediate 5-2

### 1-(6-(1H-1,2,4-Triazol-1-yl)pyridin-3-yl)ethanone

To a solution of 1-(6-bromopyridin-3-yl)ethanone 5-1 (800 mg, 4.00 mmol) and 1H-1,2,4-triazole (400 mg, 5.79 mmol) in N,N-dimethylformamide (15 mL) was added potassium carbonate (1.11 g, 8.03 mmol) at 0° C. The reaction mixture was stirred at 100° C. for 4 hours. The reaction mixture was quenched with water (20 mL) and extracted with ethyl acetate (50 mL) for three times. The combined organic layers were dried over Na₂SO₄₍ₛ₎ and filtered. The filtrate was concentrated and purified by C18 column (acetonitrile:water=5% to 100%) to give the title compound (615 mg, 100% purity from LCMS, 82% yield) as white solids. LC-MS (ESI): R_{T}=1.17 min, mass calcd. for C₉H₈N₄O 188.07, m/z found 189.1 [M+H]⁺.

### Intermediate 5-3

### 1-(6-(1H-1,2,4-Triazol-1-yl)pyridin-3-yl)ethanol

To a solution of 1-(6-(1H-1,2,4-triazol-1-yl)pyridin-3-yl)ethanone 5-2 (565 mg, 100% purity, 3.00 mmol) in methanol (6 mL) was slowly added sodium borohydride (270 mg, 7.14 mmol) at 0° C. After stirring at room temperature for 2 hours, the reaction was quenched with acetone (5 mL) dropwise and concentrated to give a residue, which was dissolved into ethyl acetate (20 mL) and washed with brine (10 mL) twice, dried over Na₂SO₄₍ₛ₎ and filtered. The filtrate was concentrated to give the title compound (545 mg, 94% purity from LCMS, 90% yield) as white solids. LC-MS (ESI): R_{T}=0.846 min, mass calcd. for C₉H₁₀N₄O 190.09, m/z found 191.2 [M+H]⁺.

### Intermediate 5-4

### 5-(1-Bromoethyl)-2-(1H-1,2,4-triazol-1-yl)pyridine

To a solution of 1-(6-(1H-1,2,4-triazol-1-yl)pyridin-3-yl)ethanol 5-3 (545 mg, 94% purity, 2.69 mmol) in dichloromethane (6 mL) was slowly added phosphorus(III) bromide (940 mg, 3.47 mmol) at 0° C. After stirring at room temperature for 2.5 hours, the reaction mixture was poured into ice water (10 mL) and adjusted with saturated sodium bicarbonate solution to pH=8˜9, extracted with dichloromethane (20 mL) for three times. The combined organic layers were washed with brine (10 mL) twice, dried over Na₂SO₄₍ₛ₎ and filtered. The filtrate was concentrated (at ˜10° C.) to give the title compound (595 mg, 70% purity from LCMS, 61% yield) as yellow oil. LC-MS (ESI): R_{T}=1.514 min, mass calcd. for C₉H₉BrN₄ 252.00, m/z found 252.9 [M+H]⁺.

### Compound 5

### (3R,7R)-9-(1-(6-(1H-1,2,4-Triazol-1-yl)pyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

(3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (260 mg, 100% purity, 0.66 mmol), 5-(1-bromoethyl)-2-(1H-1,2,4-triazol-1-yl)pyridine 5-4 (350 mg, 70% purity, 0.97 mmol) and N-benzyl-N,N-diethylethanaminium chloride (32 mg, 0.140 mmol) were mixed in 2-methyltetrahydrofuran (2.5 mL) and 50% wt. sodium hydroxide solution (2.5 mL) was added. Then the reaction mixture was stirred at room temperature under nitrogen atmosphere for 3 hours. The reaction mixture was quenched with water (10 mL) and extracted with ethyl acetate (20 mL) twice. The combined organic layer was dried over Na₂SO₄₍ₛ₎ and filtered. The filtrate was concentrated under reduced pressure to give crude, which was purified by C18 column (acetonitrile:water=5% to 90%) to give the title compound (285 mg, 100% purity from LCMS, 76% yield) as white solids. LC-MS (ESI): R_{T}=1.58 min, mass calcd. for C₂₇H₂₆Cl₂N₈O₂ 564.16, m/z found 565.4 [M+H]⁺.

### Compounds 5A and 5B

### (3R,7R)-9-((R*)-1-(6-(1H-1,2,4-Triazol-1-yl)pyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

(5A) and (3R,7R)-9-((S*)-1-(6-(1H-1,2,4-triazol-1-yl)pyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (5B) A racemic mixture of (3R,7R)-9-(1-(6-(1H-1,2,4-triazol-1-yl)pyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 5 (350 mg, 100% purity, 0.62 mmol) was separated by chiral. HPLC (separation condition: Column: Chiralpak IA 5 μm 20*250 mm; Mobile Phase: ACN:IPA=70:30 at 60 g/min; Temp: 30° C.; Wavelength: 254 nm.) to afford the title compound 5A (113.5 mg, 32% yield, 99.0% purity, 100% stereopure) as white solids and 5B (108.5 mg, 31% yield, 99.7% purity, 99.9% stereopure) as white solids.

### 5A:

LC-MS (ESI): R_{T}=2.381 min, mass calcd. for C₂₇H₂₆Cl₂N₈O₂ 564.16, m/z found 565.3 [M+H]f.

Chiral analysis (Column: Chiralpak IA 5 m 4.6*250 mm; Mobile Phase: ACN:IPA=70:30 at 1 mL/min; Col. Temp: 30° C.; Wavelength: 254 nm, R_{T}=5.965 min). ¹H NMR (400 MHz, DMSO-d₆) δ 9.37 (s, 1H), 8.57 (br s, 1H), 8.31 (s, 1H), 8.09 (br s, 1H), 7.89-7.87 (m, 1H), 7.76-7.74 (m, 2H), 7.46-7.43 (m, 1H), 5.98-5.78 (m, 1H), 5.52-5.14 (m, 1H), 4.65-4.40 (m, 2H), 4.24-4.09 (m, 1H), 3.90-3.79 (m, 1H), 3.23-3.13 (m, 1H), 2.98-2.88 (m, 1H), 2.64-2.54 (m, 1H), 1.70-1.57 (m, 3H), 1.28 (d, J=6.4 Hz, 3H), 1.19-1.08 (m, 3H).

### 5B:

LC-MS (ESI): R_{T}=2.458 min, mass calcd. for C₂₇H₂₆Cl₂N₈O₂ 564.16, m/z found 565.2 [M+H]f. Chiral analysis (Column: Chiralpak IA 5 m 4.6*250 mm; Mobile Phase: ACN:IPA=70:30 at 1 mL/min; Col. Temp: 30° C.; Wavelength: 254 nm, R_{T}=13.336 min). ¹H NMR (400 MHz, DMSO-d₆) δ 9.36 (s, 1H), 8.55 (s, 1H), 8.31 (s, 1H), 8.07 (s, 1H), 7.86 (s, 1H), 7.76-7.73 (m, 2H), 7.46-7.44 (m, 1H), 6.01-5.76 (m, 1H), 5.51-5.15 (m, 1H), 4.63-4.34 (m, 2H), 4.22-4.09 (m, 1H), 3.58-3.43 (m, 2H), 2.99-2.89 (m, 1H), 2.64-2.53 (m, 1H), 1.69-1.57 (m, 3H), 1.45 (d, J=6.0 Hz, 3H), 1.20-1.07 (m, 3H).

### Compounds 6A and 6B

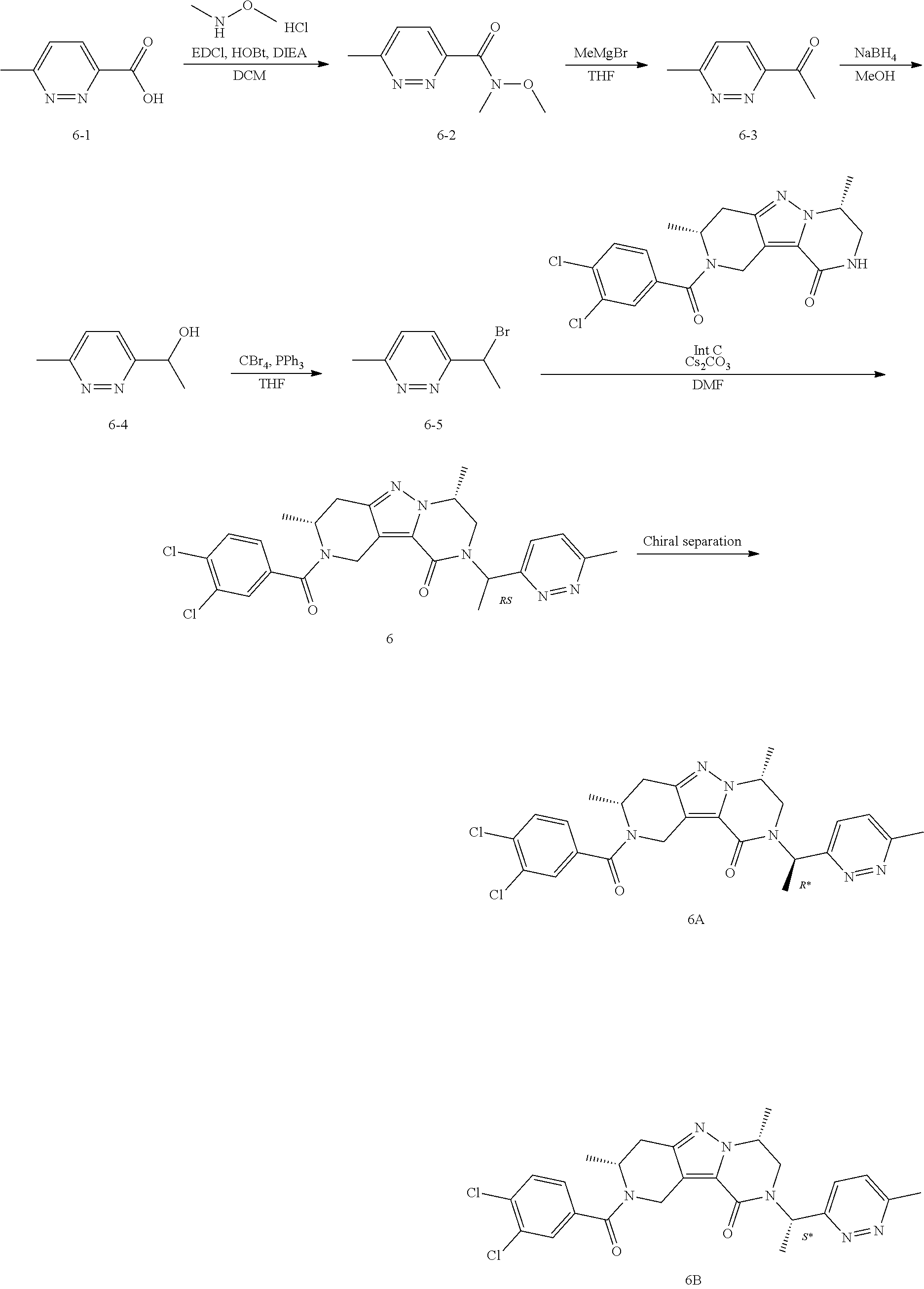

### Intermediate 6-2

### N-Methoxy-N,6-dimethylpyridazine-3-carboxamide

To a solution of 6-Methylpyridazine-3-carboxylic acid 6-1 (3.5 g, 25.340 mmol), N, O-dimethylhydroxylamine hydrochloride (3.7 g, 37.932 mmol), 1H-benzo[d][1,2,3]triazol-1-ol (4.8 g, 35.523 mmol) and triethylamine (5.3 g, 52.377 mmol) in dichloromethane (20 mL) was added 1-ethyl-3-(3-dimethylaminopropyl)carbodiimide (7.3 g, 38.080 mmol) at room temperature under nitrogen atmosphere. After stirring at room temperature overnight, the mixture was concentrated and purified by column chromatography on silica gel (petroleum ether:ethyl acetate=1:1) to give the title compound (3 g, 90% purity from ¹H NMR, 59% yield) as yellow oil. ¹H NMR (400 MHz, CDCl₃) δ 7.85-7.73 (m, 1H), 7.47-7.40 (m, 1H), 3.79 (s, 3H), 3.40 (br s, 3H), 2.78 (s, 3H).

### Intermediate 6-3

### 1-(6-Methylpyridazin-3-yl)ethanone

To the solution of N-methoxy-N,6-dimethylpyridazine-3-carboxamide 6-2 (3 g, 90% purity, 14.901 mmol) in tetrahydrofuran (20 mL) was added 1 M methylmagnesium bromide in tetrahydrofuran (19 mL, 19 mmol) at 0° C. The mixture was stirred at 0° C. for 1 hour. The mixture was quenched with ammonium chloride aqueous solution (10 mL) and extracted with ethyl acetate (10 mL) twice. The combined organic layers were washed with brine (10 mL), dried over Na₂SO₄₍ₛ₎ and filtered. The filtrate was concentrated to give the title compound (2 g, 90% purity from ¹H NMR, 89% yield) as a yellow oil. ¹H NMR (400 MHz, CDCl₃) δ 8.01 (d, J=8.8 Hz, 1H), 7.46 (d, J=8.8 Hz, 1H), 2.86 (s, 3H), 2.80 (s, 3H).

### Intermediate 6-4

### 1-(6-Methylpyridazin-3-yl)ethanol

To a solution of 1-(6-Methylpyridazin-3-yl)ethanone 6-3 (2 g, 90% purity, 13.221 mmol) in methanol (15 mL) was added sodium borohydride (750 mg, 19.824 mmol) at 0° C. After stirred at 0° C. for 1 hour, the mixture was diluted with 0.5 M hydrochloric acid aqueous solution (10 mL) and extracted with ethyl acetate (10 mL) for three times. The combined organic layers were washed with brine (10 mL), dried over Na₂SO₄₍ₛ₎ and filtered. The filtrate was concentrated and purified by silica gel column chromatography (petroleum ether:ethyl acetate=1:1) to give the title compound (1.9 g, 90% purity from ¹H NMR, 94% yield) as yellow oil. ¹H NMR (400 MHz, CDCl₃) δ 7.44 (d, J=8.8 Hz, 1H), 7.32 (d, J=8.4 Hz, 1H), 5.09 (q, J=6.8 Hz, 1H), 2.69 (s, 3H), 1.54 (d, J=6.8 Hz, 3H).

### Intermediate 6-5

### 3-(1-Bromoethyl)-6-methylpyridazine

To a solution of 1-(6-Methylpyridazin-3-yl)ethanol 6-4 (1.9 g, 90% purity, 12.376 mmol) in tetrahydrofuran (20 mL) were added triphenylphosphine (5 g, 19.063 mmol) and perbromomethane (5 g, 15.077 mmol) at 0° C. After stirred at 25° C. for 12 hours, the mixture was filtered. The filtrate was concentrated and purified by column chromatography on silica gel (petroleum ether:ethyl acetate=1:1) to give the title compound (1.5 g, 90% purity from ¹H NMR, 54% yield) as yellow oil. ¹H NMR (400 MHz, CDCl₃) δ 7.64 (d, J=8.8 Hz, 1H), 7.38 (d, J=8.0 Hz, 1H), 5.46 (q, J=6.8 Hz, 1H), 2.74 (s, 3H), 2.11 (d, J=6.8 Hz, 3H).

### Compound 6

### (3R,7R)-2-(3,4-dichlorobenzoyl)-3,7-dimethyl-9-(1-(6-methylpyridazin-3-yl)ethyl)-1,2,3,4,8,9-hexahydropyrido[4′,3′:3,4]pyrazolo[1,5-a]pyrazin-10(7H)-one

To the solution of (3R,7R)-2-(3,4-Dichlorobenzoyl)-3,7-dimethyl-1,2,3,4,8,9-hexahydropyrido[4′,3′:3,4]pyrazolo[1,5-a]pyrazin-10(7H)-one Int C (200 mg, 95% purity, 0.483 mmol) in N,N-dimethylformamide (10 mL) was added cesium carbonate (510 mg, 1.565 mmol) and 3-(1-bromoethyl)-6-methylpyridazine 6-5 (220 mg, 90% purity, 0.985 mmol). Then the mixture was stirred at 100° C. overnight. The mixture was diluted with water (10 mL) and extracted with ethyl acetate (10 mL) twice. The combined organic layers were washed with brine (10 mL), dried over Na₂SO₄₍ₛ₎ and filtered. The filtrate was concentrated and purified by silica gel column chromatography (petroleum ether:ethyl acetate=1:1) to give the title compound (210 mg, 100% purity from LCMS, 85% yield) as yellow solids. LC-MS (ESI): R_{T}=1.51 min, mass calcd. for C₂₅H₂₆Cl₂N₆O₂ 512.1, m/z found 513.2 [M+H]⁺.

### Compounds 6A and 6B

### (3R,7R)-2-(3,4-dichlorobenzoyl)-3,7-dimethyl-9-((R*)-1-(6-methylpyridazin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-((S*)-1-(6-methylpyridazin-3-yl)ethyl)-1,2,3,4,8,9-hexahydropyrido[4′,3′:3,4]pyrazolo[1,5-a]pyrazin-10(7H)-one

A racemic mixture of (3R,7R)-2-(3,4-dichlorobenzoyl)-3,7-dimethyl-9-(1-(6-methylpyridazin-3-yl)ethyl)-1,2,3,4,8,9-hexahydropyrido[4′,3′:3,4]pyrazolo[1,5-a]pyrazin-10(7H)-one 6 (210 mg, 100% purity, 0.409 mmol) was separated by chiral Prep. HPLC (separation condition: Column: Chiralpak IE 5 μm 30*250 mm; Mobile Phase: MeOH:EtOH=50:50 at 25 mL/min; Temp: 30° C.; Wavelength: 254 nm) to give 6A (56.9 mg, 96.5% purity from LCMS, 26% yield, 100% stereopure) and 6B (51.5 mg, 96.7% purity from LCMS, 24% yield, 100% stereopure) as white solids.

### 6A:

LC-MS (ESI): R_{T}=3.632 min, mass calcd. for C₂₅H₂₆Cl₂N₆O₂ 512.1, m/z found 513.1 [M+H]⁺. Chiral analysis (Column: Chiralpak IE 5 μm 4.6*250 mm; Mobile Phase: MeOH:EtOH=50:50 at 1 mL/min; Temp: 30° C.; Wavelength: 254 nm; R_{T}=9.910 min). H NMR (400 MHz, CDCl₃) δ 7.52 (d, J=2.8 Hz, 1H), 7.51-7.49 (m, 1H), 7.46-7.39 (m, 1H), 7.32-7.30 (m, 1H), 7.26-7.24 (m, 1H), 6.15-5.98 (br s, 1H), 5.75-5.28 (m, 1H), 4.88-4.20 (m, 3H), 3.93-3.89 (m, 1H), 3.59-3.54 (m, 1H), 3.16-2.90 (br s, 1H), 2.71 (s, 3H), 2.67-2.64 (m, 1H), 1.74 (d, J=6.4 Hz, 3H), 1.31 (d, J=6.4 Hz, 3H), 1.24 (m, 3H).

### 6B:

LC-MS (ESI): R_{T}=3.707 min, mass calcd. for C₂₅H₂₆Cl₂N₆O₂ 512.1, m/z found 513.2 [M+H]⁺. Chiral analysis (Column: Chiralpak IE 5 μm 4.6*250 mm; Mobile Phase: MeOH:EtOH=50:50 at 1 mL/min; Temp: 30° C.; Wavelength: 254 nm; R_{T}=13.584 min). ¹H NMR (400 MHz, CDCl₃) δ 7.52-7.49 (m, 2H), 7.46-7.35 (m, 1H), 7.31-7.29 (m, 1H), 7.24 (s, 1H), 6.16-5.99 (m, 1H), 5.80-5.30 (m, 1H), 5.30-4.80-4.21 (m, 3H), 3.79-3.75 (m, 1H), 3.63-3.51 (m, 1H), 3.12-2.87 (m, 1H), 2.70 (s, 3H), 2.64-2.58 (m, 1H), 1.75 (d, J=6.4 Hz, 3H), 1.67 (d, J=6.4 Hz, 3H), 1.26 (m, 3H).

### Compounds 7A and 7B

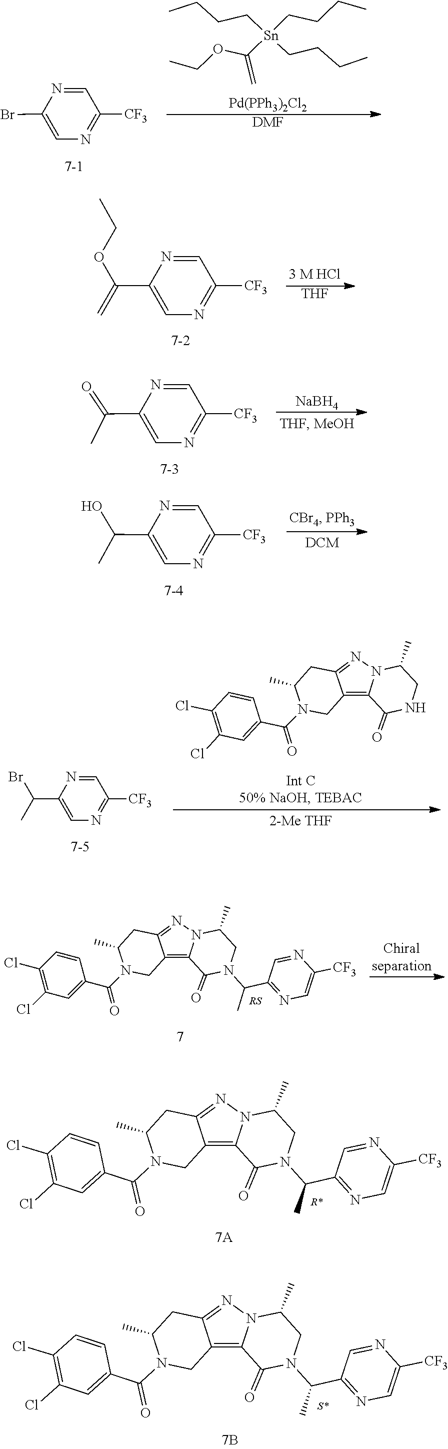

### Intermediate 7-2

### 2-(1-Ethoxyvinyl)-5-(trifluoromethyl)pyrazine

To a solution of 2-bromo-5-(trifluoromethyl)pyrazine 7-1 (1.0 g, 4.41 mmol) and tributyl(1-ethoxyvinyl)stannane (1.8 mL, 5.33 mmol) in N,N-dimethylformamide (10 mL) was added bis(triphenylphosphine)palladium(II) chloride (248 mg, 0.353 mmol) under nitrogen atmosphere. After stirring at 80° C. for 3 hours, the reaction mixture was quenched with saturated potassium fluoride (50 mL). The mixture was stirred at room temperature for 45 minutes, extracted with ethyl acetate (30 mL) for three times. The combined organic layers were washed with brine (30 mL), dried over Na₂SO₄₍ₛ₎, filtered. The filtrate was concentrated and purified by silica gel column chromatography (petroleum ether:ethyl acetate=20:1 to 10:1) to give the title compound (756 mg, 90% purity from NMR, 71% yield) as a pale yellow oil. ¹1H NMR (300 MHz, CDCl₃) δ 9.02 (s, 1H), 8.85 (s, 1H), 5.58 (s, 1H), 4.59 (s, 1H), 4.03 (q, J=6.9 Hz, 2H), 1.47 (t, J=6.9 Hz, 3H).

### Intermediate 7-3

### 1-(5-(Trifluoromethyl)pyrazin-2-yl)ethanone

To a solution of 2-(1-ethoxyvinyl)-5-(trifluoromethyl)pyrazine 7-2 (756 mg, 90% purity, 3.12 mmol) in tetrahydrofuran (6 mL) was added 3 M Hydrochloric acid in water (3.5 mL) at 0° C. After stirring at room temperature overnight, the reaction was quenched with saturated sodium carbonate to pH=9-10 at 0° C. The aqueous layer was extracted with tert-butyl methyl ether (5 mL) twice. The combined organic layers were washed with brine (5 mL), dried over Na₂SO₄₍ₛ₎ and filtered. The filtrate was concentrated to give the title compound (574 mg, 90% purity from ¹H NMR, 87% yield) as colorless solids. ¹H NMR (300 MHz, CDCl₃) δ 9.32 (s, 1H), 9.01 (s, 1H), 2.77 (s, 3H).

### Intermediate 7-4

### 1-(5-(Trifluoromethyl)pyrazin-2-yl)ethanol

To a solution of 1-(5-(trifluoromethyl)pyrazin-2-yl)ethanone 7-3 (1.2 g, 95% purity, 6.00 mmol) in tetrahydrofuran (24 mL) was added sodium tetrahydroborate (320 mg, 8.46 mmol) and methanol (6 mL). After stirred at 0° C. for 1 hour, the mixture was quenched with water, extracted with ethyl acetate (50 mL) for three times. The organic layers were combined, washed with brine (50 mL), dried over Na₂SO₄₍ₛ₎ and filtered. The filtrate was concentrated to get the title compound (1.1 g, 78% purity from LCMS, 74% yield) as yellow oil. LC-MS (ESI): R_{T}=1.063 min, mass calcd. for C₇H₇F₃N₂O 192.05, m/z found 193.2 [M+H]⁺.

### Intermediate 7-5

### 2-(1-Bromoethyl)-5-(trifluoromethyl)pyrazine

To a solution of 1-(5-(trifluoromethyl)pyrazin-2-yl)ethanol 7-4 (1.1 g, 78% purity, 4.47 mmol) in dichloromethane (22 mL) at 0° C. under nitrogen atmosphere was added carbon tetrabromide (2.5 g, 7.54 mmol) and triphenylphosphine (2.0 g, 7.63 mmol). The reaction mixture was stirred at 0° C. for 1 hour. The reaction mixture was purified by silica gel column chromatography (petroleum ether:ethyl acetate=10:1 to 5:1) to get the title compound (920 mg, 90% purity from ¹H NMR, 73% yield) as yellow oil. ¹H NMR (400 MHz, CDCl₃) δ 8.89 (s, 1H), 8.85 (s, 1H), 5.29 (q, J=6.8 Hz, 1H), 2.13 (d, J=7.2 Hz, 3H).

### Compound 7

### (3R,7R)-2-(3,4-Dichlorobenzoyl)-3,7-dimethyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (220 mg, 95% purity, 0.53 mmol), 2-(1-bromoethyl)-5-(trifluoromethyl)pyrazine 7-5 (225 mg, 90% purity, 0.79 mmol) and N-benzyl-N,N-diethylethanaminium chloride (23 mg, 0.10 mmol) in 2-methyltetrahydrofuran (2.5 mL) was added 50% wt. sodium hydroxide solution (2.5 mL). The mixture was stirred at room temperature under nitrogen atmosphere for 3 hours. Then was quenched with water (10 mL) and extracted with ethyl acetate (25 mL) twice. The combined organic layer was dried over Na₂SO₄₍ₛ₎ and filtered. The filtrate was concentrated under reduced pressure to give crude, which was purified by C18 column (acetonitrile:water=5% to 90%) to give the title compound (205 mg, 100% purity from LCMS, 68% yield) as yellow solids. LC-MS (ESI): R_{T}=1.74 min, mass calcd. for C₂₅H₂₃Cl₂F₃N₆O₂ 566.12, m/z found 567.2 [M+H]⁺.

### Compounds 7A and 7B

### (3R,7R)-2-(3,4-dichlorobenzoyl)-3,7-dimethyl-9-((R*)-1-(5-(trifluoromethyl)pyrazin-2-yl)ethyl)-1,2,3,4,8,9-hexahydropyrido[4′,3′:3,4]pyrazolo[1,5-a]pyrazin-10(7H)-one (7A) and (3R,7R)-2-(3,4-dichlorobenzoyl)-3,7-dimethyl-9-((S*)-1-(5-(trifluoromethyl)pyrazin-2-yl)ethyl)-1,2,3,4,8,9-hexahydropyrido[4′,3′:3,4]pyrazolo[1,5-a]pyrazin-10(7H)-one (7B)

A racemic mixture of (3R,7R)-2-(3,4-dichlorobenzoyl)-3,7-dimethyl-9-(1-(5-(trifluoromethyl)pyrazin-2-yl)ethyl)-1,2,3,4,8,9-hexahydropyrido[4′,3′:3,4]pyrazolo[1,5-a]pyrazin-10(7H)-one 7 (300 mg, 100% purity, 0.53 mmol) was separated by chiral. HPLC (separation condition: Column: Chiralpak IE 5 μm 20*250 mm; Mobile Phase: Hex:EtOH=30:70 at 60 g/min; Temp: 30° C.; Wavelength: 254 nm.) to afford the title compound 7A (61.1 mg, 20.3% yield, 99.7% purity, 100% stereopure) as white solids and compound 7B (72.9 mg, 24.2% yield, 99.6% purity, 99.9% stereopure) as white solids.

### 7A:

LC-MS (ESI): R_{T}=3.865 min, mass calcd. for C₂₅H₂₃Cl₂F₃N₆O₂ 566.1, m/z found 567.2 [M+H]⁺. Chiral analysis (Column: Chiralpak IE 5 m 4.6*250 mm; Mobile Phase: Hex:EtOH=30:70 at 1 mL/min; Col. Temp: 30° C.; Wavelength: 254 nm. R_{T}=7.012 min). ¹H NMR (400 MHz, DMSO-d₆) δ 9.17 (s, 1H), 9.00-8.87 (m, 1H), 7.75-7.73 (m, 2H), 7.45-7.42 (m, 1H), 5.94-5.84 (m, 1H), 5.46-5.20 (m, 1H), 4.50 (s, 2H), 4.18-3.92 (m, 2H), 3.42 (s, 1H), 2.96-2.91 (m, 1H), 2.63-2.55 (m, 1H), 1.74-1.58 (m, 3H), 1.36 (d, J=6.4 Hz, 3H), 1.17-1.11 (m, 3H). ¹⁹F NMR (376 MHz, DMSO-d₆) δ −66.1.

### 7B:

LC-MS (ESI): R_{T}=3.838 min, mass calcd. for C₂₅H₂₃Cl₂F₃N₆O₂ 566.12, m/z found 567.2 [M+H]⁺. Chiral analysis (Column: Chiralpak IE 5 m 4.6*250 mm; Mobile Phase: Hex:EtOH=30:70 at 1 mL/min; Col. Temp: 30° C.; Wavelength: 254 nm. R_{T}=8.890 min). ¹H NMR (400 MHz, DMSO-d₆) δ 9.16 (s, 1H), 8.94 (br, s, 1H), 7.74-7.72 (m, 2H), 7.45-7.42 (m, 1H), 6.07-5.81 (m, 1H), 5.46-5.15 (m, 1H), 4.49 (br s, 2H), 4.23-4.07 (m, 1H), 3.74-3.56 (m, 2H), 2.96-2.91 (m, 1H), 2.64-2.56 (m, 1H), 1.72-1.58 (m, 3H), 1.47 (d, J=6.4 Hz, 3H), 1.21-1.07 (m, 3H). ¹⁹F NMR (376 MHz, DMSO-d₆) δ −66.1.

### Compounds 8A and 8B

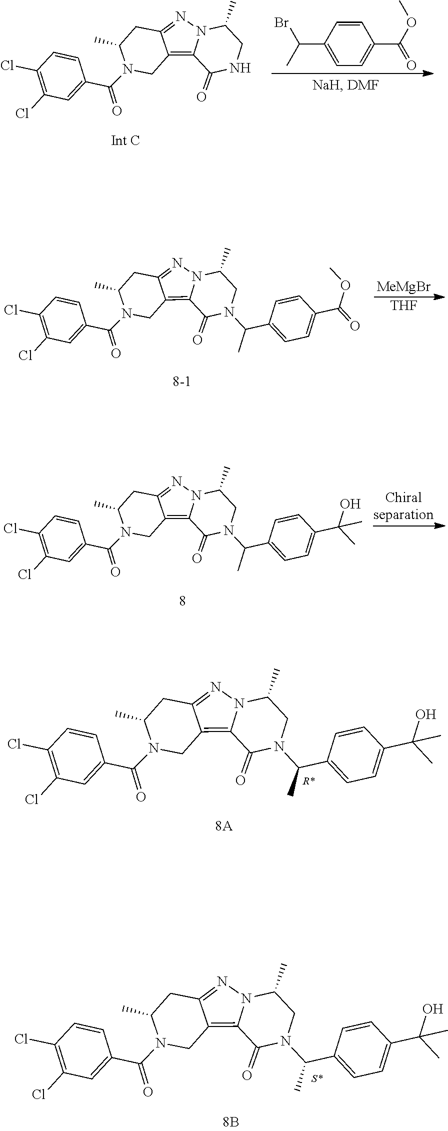

### Intermediate 8-1

### Methyl 4-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzoate

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (500 mg, 95% purity, 1.21 mmol) in N, N-dimethylformamide (5 mL) was added 60% wt. sodium hydride in mineral oil (250 mg, 6.25 mmol). After stirring at 0° C. for 0.5 hour, methyl 4-(1-bromoethyl)benzoate (550 mg, 2.26 mmol) was added into the mixture. After stirring at 0° C. for 1 hour, the mixture was diluted with water (50 mL) and extracted with ethyl acetate (30 mL) twice. The combined organic layers were washed with brine (30 mL), dried over Na₂SO₄₍ₛ₎ and filtered. The filtrate was concentrated under reduced pressure to give the crude compound, which was purified by C18 column (acetonitrile:water=30% to 80%) to give the title compound (400 mg, 90% purity from ¹H NMR, 53.7% yield) as white solids. LC-MS (ESI): R_{T}=1.74 min, mass calcd. for C₂₈H₂₈Cl₂N₄O₄ 554.2, m/z found 555.2 [M+H]⁺. ¹H NMR (400 MHz, CDCl₃) δ 8.05-8.01 (m, 2H), 7.55-7.52 (m, 2H), 7.52-7.50 (m, 2H), 7.28-7.25 (m, 1H), 6.11 (br s, 1H), 5.84-5.38 (m, 1H), 4.93-4.25 (m, 3H), 3.92 (d, J=1.2 Hz, 3H), 3.68-3.57 (m, 1H), 3.31-2.92 (m, 2H), 2.74-2.61 (m, 1H), 1.63-1.52 (m, 6H), 1.28-1.24 (m, 3H).

### Compound 8

### (3R,7R)-2-(3,4-Dichlorobenzoyl)-9-(1-(4-(2-hydroxypropan-2-yl)phenyl)ethyl)-3,7-dimethyl-1,2,3,4,8,9-hexahydropyrido[4′,3′:3,4]pyrazolo[1,5-a]pyrazin-10(7H)-one

To a solution of methyl 4-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzoate 8-1 (400 mg, 90% purity, 0.65 mmol) in tetrahydrofuran (12 mL) was added 1 M methylmagnesium bromide (5 mL, 5 mmol) in tetrahydrofuran at 0° C. After stirring at 0° C. for 0.5 hour, the mixture was diluted with water (30 mL) and extracted with ethyl acetate (30 mL) twice. The combined organic layers were washed with brine (30 mL), dried over Na₂SO₄₍ₛ₎ and filtered. The filtrate was concentrated under reduced pressure to give the crude compound, then purified by C18 column (acetonitrile:water=30% to 80%) to give the title compound (160 mg, 90% purity from ¹H NMR, 40.0% yield) as white solids. LC-MS (ESI): R_{T}=1.65 min, mass calcd. for C₂₉H₃₂Cl₂N₄O₃ 554.2, m/z found 555.3 [M+H]⁺.

### Compounds 8A and 8B

### (3R,7R)-2-(3,4-dichlorobenzoyl)-9-((R*)-1-(4-(2-hydroxypropan-2-yl)phenyl)ethyl)-3,7-dimethyl-1,2,3,4,8,9-hexahydropyrido[4′,3′:3,4]pyrazolo[1,5-a]pyrazin-10(7H)-one (8A) and (3R,7R)-2-(3,4-dichlorobenzoyl)-9-((S*)-1-(4-(2-hydroxypropan-2-yl)phenyl)ethyl)-3,7-dimethyl-1,2,3,4,8,9-hexahydropyrido[4′,3′:3,4]pyrazolo[1,5-a]pyrazin-10(7H)-one (8B)

A racemic mixture of (3R,7R)-2-(3,4-dichlorobenzoyl)-9-(1-(4-(2-hydroxypropan-2-yl)phenyl)ethyl)-3,7-dimethyl-1,2,3,4,8,9-hexahydropyrido[4′,3′:3,4]pyrazolo[1,5-a]pyrazin-10(7H)-one 8 (160 mg, 90% purity, 0.26 mmol) was separated by chiral Prep. HPLC (separation condition: Column: Chiralpak IC 5 μm 20*250 mm; Mobile Phase: Hex:EtOH=30:70 at 25 mL/min; Temp: 30° C.; Wavelength: 254 nm) to give the title compounds 8A (55 mg, 99.9% purity from LCMS, 38.2% yield, 100% stereopure) and 8B (50 mg, 99.8% purity from LCMS, 34.7% yield, 99.9% stereopure) as white solids.

### 8A:

LC-MS (ESI): R_{T}=3.949 min, mass calcd. for C₂₉H₃₂Cl₂N₄O₃ 554.2, m/z found 537.3 [M-18]⁺. Chiral analysis (Column: Chiralpak IC 5 m 4.6*250 mm; Mobile Phase: Hex:EtOH=30:70 at 1 mL/min; Col. Temp: 30° C.; Wavelength: 254 nm; R_{T}=9.584 min). ¹H NMR (400 MHz, CDCl₃) δ 7.53-7.48 (m, 4H), 7.34-7.32 (m, 2H), 7.27-7.25 (m, 1H), 6.05 (br s, 1H), 5.75-5.38 (m, 1H), 4.94-4.36 (m, 3H), 3.66-3.53 (m, 1H), 3.12-2.96 (m, 2H), 2.72-2.59 (m, 1H), 1.73 (s, 1H), 1.58-1.57 (m, 9H), 1.26-1.23 (m, 6H).

### 8B:

LC-MS (ESI): R_{T}=3.963 min, mass calcd. for C₂₉H₃₂Cl₂N₄O₃ 554.2, m/z found 537.3 [M-18]⁺. Chiral analysis (Column: Chiralpak IC 5 m 4.6*250 mm; Mobile Phase: Hex:EtOH=30:70 at 1 mL/min; Col. Temp: 30° C.; Wavelength: 254 nm; R_{T}=14.755 min). ¹H NMR (400 MHz, CDCl₃) δ 7.54-7.46 (m, 4H), 7.30-7.26 (m, 3H), 6.03 (br s, 1H), 5.81-5.33 (m, 1H), 4.97-4.21 (m, 3H), 3.33-3.26 (m, 2H), 3.12-2.92 (m, 1H), 2.74-2.59 (m, 1H), 1.72-1.58 (m, 10H), 1.52 (d, J=6.8 Hz, 3H), 1.35-1.18 (m, 3H).

### Compounds 9A and 9B

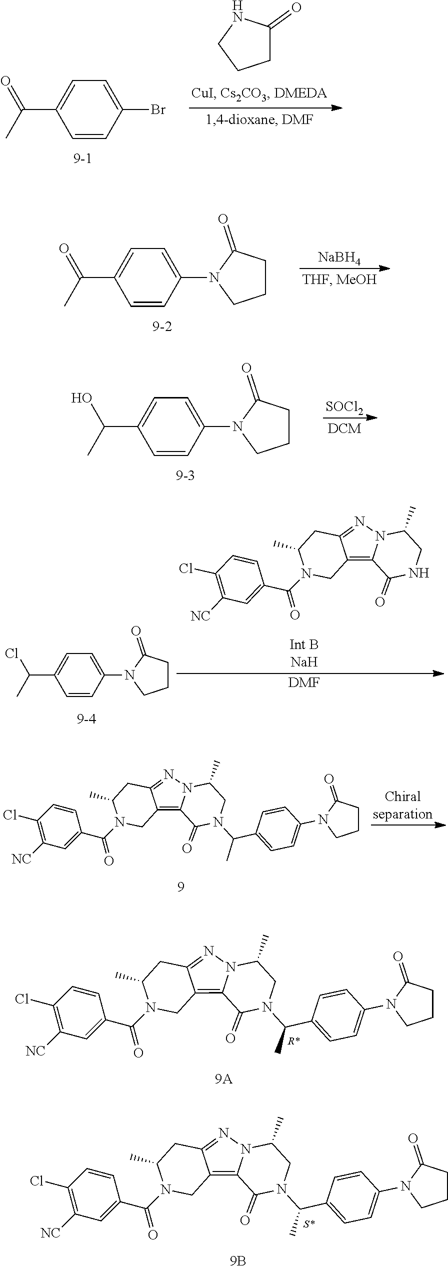

### Intermediate 9-2

### 1-(4-Acetylphenyl)pyrrolidin-2-one

To the solution of pyrrolidin-2-one (3.00 g, 35.3 mmol) in 1,4-dioxane (40 mL) and N, N-dimethylformamide (10 mL) was added 1-(4-bromophenyl)ethanone 9-1 (9.50 g, 47.7 mmol), cesium carbonate (22.0 g, 67.5 mmol), copper (I) iodide (1.50 g, 7.88 mmol) and N,N′-dimethyl-1,2-ethanediamine (650 mg, 7.37 mmol) at 25° C. The mixture was stirred at 110° C. under nitrogen atmosphere for 48 hours. The mixture was added into water (100 mL) and extracted with dichloromethane (300 mL) twice. The combined organic layer was washed with brine (100 mL), dried over Na₂SO₄₍ₛ₎ and filtered. The filtrate was concentrated under reduced pressure to get a residue, then purified by silica gel column chromatography (petroleum ether:ethyl acetate=3:1) to give the title compound (5 g, 95% purity from ¹H NMR, 66% yield) as yellow solids. LC-MS (ESI): R_{T}=1.095 min, mass calcd. for C₁₂H₁₃NO₂, 203.1, m/z found 204.1 [M+H]⁺. ¹H NMR (400 MHz, DMSO-d₆) δ 7.99-7.95 (m, 2H), 7.83-7.80 (m, 2H), 3.90-3.87 (m, 2H), 2.56-2.52 (m, 5H), 2.12-2.04 (m, 2H).

### Intermediate 9-3

### 1-(4-(1-Hydroxyethyl)phenyl)pyrrolidin-2-one

To a solution of 1-(4-acetylphenyl)pyrrolidin-2-one 9-2 (1.70 g, 95% purity, 7.95 mmol) in tetrahydrofuran (9 mL) and methanol (3 mL) was slowly added sodium borohydride (200 mg, 5.29 mmol) at 0° C. After stirring at room temperature for 2 hours, the reaction was quenched with acetone (3 mL) dropwise and concentrated to give the title compound (1.5 g, 95% purity from ¹H NMR, 87% yield) as yellow solids. LC-MS (ESI): R_{T}=1.004 min, mass calcd. for C₁₂H₁₅NO₂, 205.1, m/z found 206.0 [M+H]⁺. ¹H NMR (400 MHz, DMSO-d₆) δ 7.57 (d, J=8.8 Hz, 2H), 7.32 (d, J=8.4 Hz, 2H), 5.10 (d, J=4.0 Hz, 1H), 4.72-4.66 (m, 1H), 3.83-3.79 (m, 2H), 2.49-2.45 (m, 2H), 2.08-2.01 (m, 2H), 1.30 (d, J=2.4 Hz, 3H).

### Intermediate 9-4

### 1-(4-(1-Chloroethyl)phenyl)pyrrolidin-2-one

To a solution of 1-(4-(1-Hydroxyethyl)phenyl)pyrrolidin-2-one 9-3 (1.40 g, 95% purity, 6.48 mmol) in dichloromethane (10 mL) was slowly added sulfurous dichloride (1.60 g, 13.449 mmol) at 0° C. After stirring at room temperature for 6 hours, the reaction mixture was poured into ice water (30 mL) and extracted with dichloromethane (50 mL) twice. The combined organic layers were washed with saturated sodium bicarbonate solution (30 mL) twice and brine (30 mL) twice, dried over Na₂SO₄₍ₛ₎ and filtered. The filtrate was concentrated under reduced pressure to give title crude compound (750 mg, 85% purity from ¹H NMR, 44% yield) as yellow solids. ¹H NMR (400 MHz, DMSO-d₆) δ 7.66-7.63 (m, 2H), 7.47 (d, J=8.4 Hz, 2H), 5.34 (q, J=6.8 Hz, 1H), 3.85-3.81 (m, 2H), 2.50-2.47 (m, 2H), 2.10-2.02 (m, 2H), 1.79 (d, J=6.4 Hz, 3H).

### Compound 9

### 2-Chloro-5-((3R,7R)-3,7-dimethyl-10-oxo-9-(1-(4-(2-oxopyrrolidin-1-yl)phenyl)ethyl)-1,2,3,4,7,8,9,10-octahydropyrido[4′,3′:3,4]pyrazolo[1,5-a]pyrazine-2-carbonyl)benzonitrile

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (300 mg, 100% purity, 0.78 mmol) in N, N-dimethylformamide (4 mL) was added sodium hydride (66 mg, 60% purity, 1.65 mmol) at 0° C. and stirred at 0° C. for 10 minutes. Then 1-(4-(1-chloroethyl)phenyl)pyrrolidin-2-one 9-4 (330 mg, 85% purity, 1.254 mmol) was added and stirred at 35° C. for 2 hours. The mixture was added into water (5 mL) and extracted with dichloromethane (15 mL) twice. The combined organic layer was washed with water (10 mL), brine (10 mL), dried over Na₂SO₄₍ₛ₎ and filtered. The filtrate was concentrated under reduced pressure to get a residue, which was purified by C18 column (acetonitrile:water=5% to 80%) to give the title compound (300 mg, 99% purity from LCMS, 67% yield) as white solids. LC-MS (ESI): R_{T}=1.338 min, mass calcd. for C₃₁H₃₁ClN₆03, 570.2, m/z found 571.3 [M+H]⁺. ¹H NMR (400 MHz, DMSO-d₆) δ 7.75 (d, J=3.2 Hz, 1H), 7.64-7.57 (m, 4H), 7.37-7.31 (m, 2H), 6.00-5.43 (m, 2H), 4.83-4.23 (m, 3H), 3.88-3.84 (m, 2H), 3.27-2.98 (m, 2H), 2.71-2.60 (m, 3H), 2.21-2.14 (m, 2H), 1.58-1.51 (m, 4H), 1.34-1.24 (m, 6H).

### Compounds 9A and 9B

### 2-Chloro-5-((3R,7R)-3,7-Dimethyl-10-oxo-9-((R*)-1-(4-(2-oxopyrrolidin-1-yl)phenyl)ethyl)-1,2,3,4,7,8,9,10-octahydropyrido[4′,3′:3,4]pyrazolo[1,5-a]pyrazine-2-carbonyl)benzonitrile (9A) and 2-Chloro-5-((3R,7R)-3,7-dimethyl-10-oxo-9-((S*)-1-(4-(2-oxopyrrolidin-1-yl)phenyl)ethyl)-1,2,3,4,7,8,9,10-octahydropyrido[4′,3′:3,4]pyrazolo[1,5-a]pyrazine-2-carbonyl)benzonitrile (9B)

A racemate of 2-chloro-5-((3R,7R)-3,7-dimethyl-10-oxo-9-(1-(4-(2-oxopyrrolidin-1-yl)phenyl)ethyl)-1,2,3,4,7,8,9,10-octahydropyrido[4′,3′:3,4]pyrazolo[1,5-a]pyrazine-2-carbonyl)benzonitrile 9 (300 mg, 99% purity, 0.520 mmol) was separated by chiral Prep. HPLC separation condition: (Column: Chiralpak IC 5 m 30*250 mm; Mobile Phase: MeOH:DCM=70:30 at 25 mL/min; Temp: 30° C.; Wavelength: 254 nm) to give the crude chiral isomers, which were purified by C18 column (acetonitrile:water (0.1% ammonium bicarbonate)=5% to 80%) to give the title compounds 9A (130 mg, 99.5% purity, 44% yield, 100% stereopure) and 9B (130 mg, 98.6% purity, 43% yield, 99.8% stereopure) as white solids.

### 9A:

LC-MS (ESI): R_{T}=3.560 min, mass calcd. for C₃₁H₃₁ClN₆03, 570.2, m/z found 571.3 [M+H]⁺. Chiral analysis (Column: Chiralpak IC 5 m 4.6*250 mm; Mobile Phase: MeOH:DCM=70:30 at 1 mL/min; Temp: 30° C.; Wavelength: 254 nm, R_{T}=9.841 min). ¹H NMR (400 MHz, CDCl₃) δ 7.74 (s, 1H), 7.64-7.57 (m, 4H), 7.36 (d, J=7.6 Hz, 2H), 6.03 (br s, 1H), 5.73-5.42 (m, 1H), 4.78-4.35 (m, 3H), 3.88-3.84 (m, 2H), 3.57 (d, J=12.0 Hz, 1H), 3.10-2.98 (m, 2H), 2.70-2.60 (m, 3H), 2.21-2.14 (m, 2H), 1.57-1.55 (m, 3H), 1.30-1.26 (m, 6H).

### 9B:

LC-MS (ESI): R_{T}=3.922 min, mass calcd. for C₃₁H₃₁ClN₆03, 570.2, m/z found 571.3 [M+H]⁺. Chiral analysis (Column: Chiralpak IC 5 m 4.6*250 mm; Mobile Phase: MeOH:DCM=70:30 at 1 mL/min; Temp: 30° C.; Wavelength: 254 nm, R_{T}=11.616 min). ¹H NMR (400 MHz, CDCl₃) δ 7.75 (s, 1H), 7.62-7.58 (m, 4H), 7.32 (d, J=7.2 Hz, 2H), 6.01 (br s, 1H), 5.78-5.40 (m, 1H), 4.87-4.24 (m, 3H), 3.88-3.84 (m, 2H), 3.30-3.25 (m, 2H), 3.09 (br s, 1H), 2.72-2.68 (m, 1H), 2.64-2.60 (m, 2H), 2.21-2.14 (m, 2H), 1.58-1.57 (m, 3H), 1.52 (d, J=6.4 Hz, 3H) 1.30-1.28 (m, 3H).

### Compounds 10A and 10B

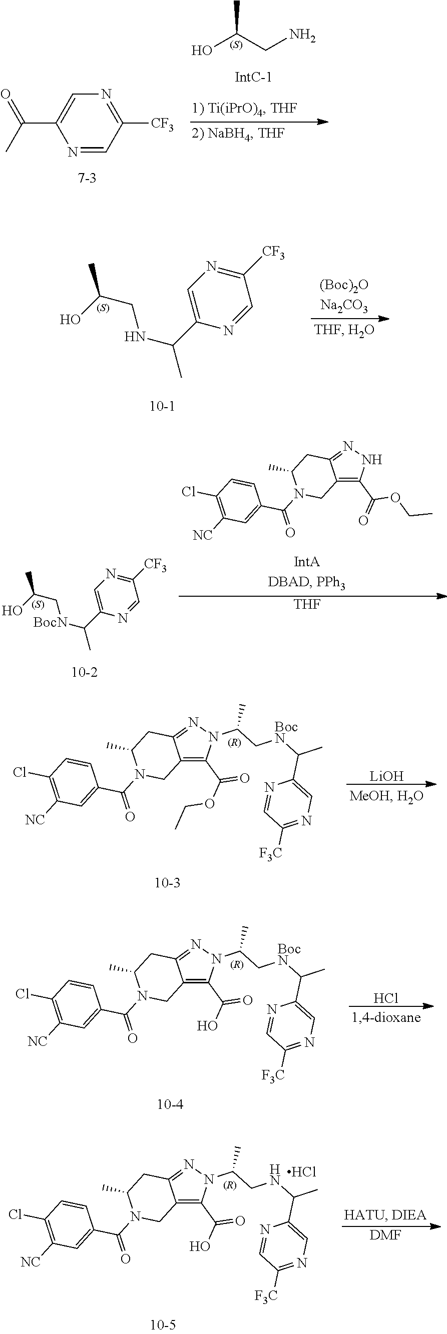

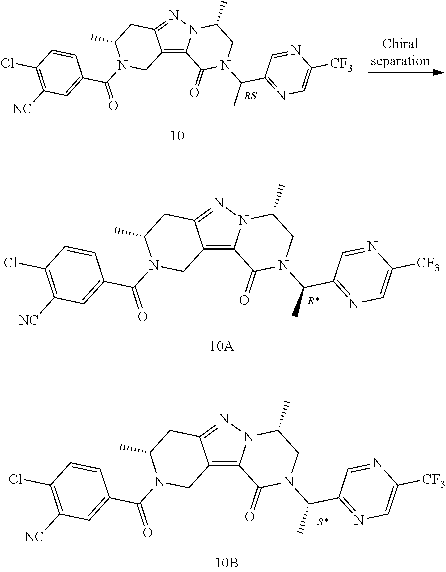 [SMILES: CC(c1cnc(C(F)(F)F)cn1)N1C[C@@H](C)n2nc3c(c2C1=O)CN(C(=O)c1ccc(Cl)c(C#N)c1)[C@H](C)C3 ; C[C@@H]1Cc2nn3c(c2CN1C(=O)c1ccc(Cl)c(C#N)c1)C(=O)N([C@H](C)c1cnc(C(F)(F)F)cn1)C[C@H]3C ; C[C@@H]1Cc2nn3c(c2CN1C(=O)c1ccc(Cl)c(C#N)c1)C(=O)N([C@@H](C)c1cnc(C(F)(F)F)cn1)C[C@H]3C]

### Intermediate 10-1

### (2S)-1-((1-(5-(Trifluoromethyl) pyrazin-2-yl) ethyl) amino) propan-2-ol

To a solution of 1-(5-(trifluoromethyl) pyrazin-2-yl) ethanone 7-3 (170 mg, 0.81 mmol, purity 90%) and (S)-(+)-1-Amino-2-propanol IntC-1 (250 mg, 3.33 mmol) in tetrahydrofuran (5 mL) was added titanium (IV) propan-2-olate (0.5 mL, 1.71 mmol). After stirring at 30° C. for 16 hours, sodium borohydride (30 mg, 0.793 mmol) was added and stirred at room temperature for 1 hour. The reaction was quenched with methanol (2 mL), poured into water (2 mL), filtered with celite. The cake was washed with ethyl acetate (30 mL). The filtrate was concentrated and purified by silica gel column chromatography (petroleum ether:ethyl acetate=3:1) to give the title compound (70 mg, 35% yield, 100% purity) as yellow oil. LC-MS (ESI): R_{T}=1.29 min, mass calcd. for C₁₀H₁₄F₃N₃O 249.1, m/z found 250.1 [M+H]⁺.

### Intermediate 10-2

### tert-Butyl ((S)-2-hydroxypropyl) (1-(5-(trifluoromethyl) pyrazin-2-yl) ethyl) carbamate

To the solution of (2S)-1-((1-(5-(trifluoromethyl) pyrazin-2-yl) ethyl) amino) propan-2-ol 10-1 (70 mg, 100% purity, 0.281 mmol) in tetrahydrofuran (2 mL) and water (2 mL) was added sodium carbonate (60 mg, 0.566 mmol) at 0° C. After stirring at 0° C. for 1 hour, di-tert-butyl dicarbonate (90 mg, 0.412 mmol) was added. The stirring was continued at 0° C. for 2 hours. The mixture was diluted with water (10 mL), concentrated at room temperature under reduced pressure to remove the volatile. The remained aqueous layer was acidified with 1 M hydrochloride aqueous solution (1 mL) and extracted with dichloromethane (10 mL) twice and brine (10 mL), dried over Na₂SO₄₍ₛ₎ and filtered. The filtrate was concentrated and purified by C18 column (acetonitrile:water (+0.02% ammonium acetate)=5% to 100%) to give the title compound (80 mg, 90% purity, 73% yield) as yellow oil. LC-MS (ESI): R_{T}=1.71 min, mass calcd. for C₁₅H₂₂F₃N₃O₃ 349.2, m/z found 294.1 [M+H-56]⁺.

### Intermediate 10-3

### (6R)-Ethyl 2-((2R)-1-((tert-butoxycarbonyl)(1-(5-(trifluoromethyl)pyrazin-2-yl)ethyl)amino)propan-2-yl)-5-(4-chloro-3-cyanobenzoyl)-6-methyl-4,5,6,7-tetrahydro-2H-pyrazolo[4,3-c]pyridine-3-carboxylate

Tert-butyl ((S)-2-hydroxypropyl) (1-(5-(trifluoromethyl) pyrazin-2-yl) ethyl) carbamate 10-2 (80 mg, 0.206 mmol, 90% purity), (R)-Ethyl 5-(4-chloro-3-cyanobenzoyl)-6-methyl-4,5,6,7-tetrahydro-2H-pyrazolo[4,3-c]pyridine-3-carboxylate Int A (150 mg, 0.382 mmol, purity 95%), triphenylphosphine (110 mg, 0.419 mmol) and di-tert-butyl diazene-1,2-dicarboxylate (90 mg, 0.391 mmol) were dissolved in tetrahydrofuran (5 mL) at 0° C. After stirring at 50° C. for 4 hours, the mixture was concentrated under reduced pressure to give crude. The crude was purified by C18 column (acetonitrile:water=5% to 100%) to give the title compound (100 mg, 69% yield, 100% purity) as white solids. LC-MS (ESI): R_{T}=1.93 min and 1.96 min, mass calcd. for C₃₃H₃₇ClF₃N₇O₅ 703.2, m/z found 704.5 [M+H]⁺.

### Intermediate 10-4

### (6R)-2-((2R)-1-((tert-Butoxycarbonyl)(1-(5-(trifluoromethyl)pyrazin-2-yl)ethyl)amino)propan-2-yl)-5-(4-chloro-3-cyanobenzoyl)-6-methyl-4,5,6,7-tetrahydro-2H-pyrazolo[4,3-c]pyridine-3-carboxylic acid

To the solution of (6R)-ethyl 2-((2R)-1-((tert-butoxycarbonyl)(1-(5-(trifluoromethyl)pyrazin-2-yl)ethyl)amino)propan-2-yl)-5-(4-chloro-3-cyanobenzoyl)-6-methyl-4,5,6,7-tetrahydro-2H-pyrazolo[4,3-c]pyridine-3-carboxylate 10-3 (100 mg, 100% purity, 0.14 mmol) in methanol (3 mL) and water (1 mL) was added lithium hydroxide hydrate (25 mg, 0.60 mmol) under nitrogen atmosphere at 0° C. After stirring at room temperature for 2 hours, the mixture was diluted with water (5 mL), concentrated at room temperature under reduced pressure to remove the volatile. The remained aqueous layer was acidified with 1 M hydrochloride aqueous solution (1 mL) and extracted with ethyl acetate (20 mL) twice and brine (20 mL), dried over Na₂SO₄₍ₛ₎ and filtered. The filtrate was concentrated to give the title compound (100 mg, 96% yield, 92% purity) as yellow oil. LC-MS (ESI): R_{T}=1.63 min and 1.67 min, mass calcd. for C₃₁H₃₃ClF₃N₇O₅ 675.2, m/z found 674.3 [M−H]⁻.

### Intermediate 10-5

### (6R)-5-(4-Chloro-3-cyanobenzoyl)-6-methyl-2-((2R)-1-((1-(5-(trifluoromethyl)pyrazin-2-yl)ethyl)amino)propan-2-yl)-4,5,6,7-tetrahydro-2H-pyrazolo[4,3-c]pyridine-3-carboxylic acid hydrochloride

A solution of (6R)-2-((2R)-1-((tert-butoxycarbonyl)(1-(5-(trifluoromethyl)pyrazin-2-yl)ethyl)amino)propan-2-yl)-5-(4-chloro-3-cyanobenzoyl)-6-methyl-4,5,6,7-tetrahydro-2H-pyrazolo[4,3-c]pyridine-3-carboxylic acid 10-4 (100 mg, 0.136 mmol, 92% purity) in 4 M hydrochloride in 1,4-dioxane (3 mL, 12 mmol) was stirred at 25° C. for 3 hours. Then the mixture was concentrated to give the title compound (90 mg, 91% purity, 98% yield) as light yellow solids. LC-MS (ESI): R_{T}=1.33 min, mass calcd. for C₂₆H₂₅ClF₃N₇O₃ 575.2, m/z found 576.2 [M+H]⁺.

### Compound 10

### 2-Chloro-5-((3R,7R)-3,7-dimethyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-2-carbonyl)benzonitrile (6R)-5-(4-chloro-3-cyanobenzoyl)-6-methyl-2-((2R)-1-((1-(5-(trifluoromethyl)pyrazin-2-yl)ethyl)amino)propan-2-yl)-4,5,6,7-tetrahydro-2H-pyrazolo[4,3-c]pyridine-3-carboxylic acid

10-5 (90 mg, 91% purity, 0.134 mmol), N-ethyl-N-isopropylpropan-2-amine (0.1 mL, 0.604 mmol) and O-(7-Azabenzotriazol-1-yl)-N,N,N′,N′-tetramethyluronium hexafluorophosphate (100 mg, 0.263 mmol) were mixed in N,N-dimethylformamide (3 mL) at 0° C. After stirring at 30° C. for 1 hour, the mixture was concentrated under reduced pressure to give crude. The crude was purified by C18 column (acetonitrile:water=5% to 100%) to give the title compound (60 mg, 80% yield, 100% purity) as white solids. LC-MS (ESI): R_{T}=1.66 min, mass calcd. for C₂₆H₂₃ClF₃N₇O₂ 557.2, m/z found 558.2 [M+H]⁺.

### Compounds 10A and 10B

### 2-Chloro-5-((3R,7R)-3,7-dimethyl-10-oxo-9-((R*)-1-(5-(trifluoromethyl)pyrazin-2-yl)ethyl)-1,2,3,4,7,8,9,10-octahydropyrido[4′,3′:3,4]pyrazolo[1,5-a]pyrazine-2-carbonyl)benzonitrile (10A) and 2-Chloro-5-((3R,7R)-3,7-dimethyl-10-oxo-9-((S*)-1-(5-(trifluoromethyl)pyrazin-2-yl)ethyl)-1,2,3,4,7,8,9,10-octahydropyrido[4′,3′:3,4]pyrazolo[1,5-a]pyrazine-2-carbonyl)benzonitrile (10B)

A racemic mixture of 2-chloro-5-((3R,7R)-3,7-dimethyl-10-oxo-9-(1-(5-(trifluoromethyl)pyrazin-2-yl)ethyl)-1,2,3,4,7,8,9,10-octahydropyrido[4′,3′:3,4]pyrazolo[1,5-a]pyrazine-2-carbonyl)benzonitrile 10 (65 mg, 0.116 mmol, 100% purity) was separated by chiral HPLC (separation condition: Column: Chiralpak IC 5 m 25*250 mm, MeOH:EtOH=50:50, 25 ml/min; Col. Temp: 30° C.; Wavelength: 254 nm) to afford the title compounds 10A (15 mg, 93.0% purity, yield 21.5%) and 10B (25 mg, 99.9% purity, 38% yield) as white solids.

### 10A:

LC-MS (ESI): R_{T}=3.287 min, mass calcd. for C₂₆H₂₃ClF₃N₇O₂ 557.2, m/z found 558.1 [M+H]⁺. Chiral analysis (Column: Chiralpak IC 5 m 4.6*250 mm; Mobile Phase: MeOH:EtOH=50:50 at 1 mL/min; Temp: 30° C.; Wavelength: 254 nm, R_{T}=8.384 min). ¹H NMR (400 MHz, DMSO-d₆) δ 8.90 (s, 1H), 8.79 (s, 1H), 7.73 (s, 1H), 7.59 (s, 2H), 6.18-6.04 (m, 1H), 5.66-4.24 (m, 4H), 3.89-3.85 (m, 1H), 3.50-3.45 (m, 1H), 3.15-2.94 (m, 1H), 2.70-2.66 (m, 1H), 1.69 (d, J=7.2 Hz, 3H), 1.42 (d, J=6.4 Hz, 3H), 1.27-1.25 (m, 3H). ¹⁹F NMR (400 MHz, CDCl₃) δ −67.56.

### 10B:

LC-MS (ESI): R_{T}=3.308 min, mass calcd. for C₂₆H₂₃ClF₃N₇O₂ 557.2, m/z found 558.1 [M+H]⁺. Chiral analysis (Column: Chiralpak IC 5 m 4.6*250 mm; Mobile Phase: MeOH:EtOH=50:50 at 1 mL/min; Temp: 30° C.; Wavelength: 254 nm, R_{T}=15.486 min). ¹H NMR (400 MHz, DMSO-d₆) δ 8.89 (s, 1H), 8.76 (s, 1H), 7.73 (s, 1H), 7.58 (s, 2H), 6.20-6.06 (m, 1H), 5.67-4.28 (m, 4H), 3.74-3.70 (m, 1H), 3.59-3.54 (m, 1H), 3.13-2.95 (m, 1H), 2.72-2.67 (m, 1H), 1.70 (d, J=7.2 Hz, 3H), 1.61 (d, J=6.8 Hz, 3H), 1.28-1.27 (m, 3H). ¹⁹F NMR (376 MHz, DMSO-d₆) δ −67.55.

### Compounds 11A and 11B

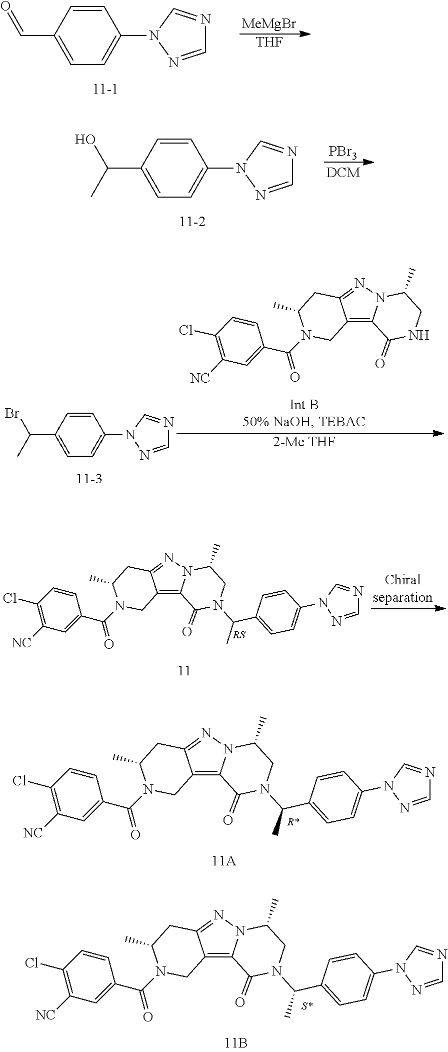

### Intermediate 11-2

### 1-(4-(1H-1,2,4-triazol-1-yl)phenyl)ethanol

To a solution of 4-(1H-1,2,4-triazol-1-yl)benzaldehyde 11-1 (500 mg, 2.89 mmol) in dry tetrahydrofuran (6 mL) was added 1 M methylmagnesium bromide in tetrahydrofuran (4 mL, 4 mmol) dropwise at 0° C. under nitrogen atmosphere, and the mixture was stirred at 20° C. for 12 hours. The reaction mixture was quenched with saturated ammonium chloride solution (10 mL), extracted with acetate (10 mL) for three times, dried over Na₂SO₄₍ₛ₎, filtered. The filtrate was concentrated to get a residue, then purified by column chromatography on silica gel (petroleum ether:ethyl acetate=10:1 to 2:1) to give the title product (400 mg, 93% purity from LCMS, 68.1% yield) as white solids. LC-MS (ESI): R_{T}=1.09 min, mass calcd. for C₁₀H₁₁N₃O 189.1 m/z found 190.2 [M+H]⁺. ¹H NMR (400 MHz, CDCl₃) δ 8.54 (s, 1H), 8.10 (s, 1H), 7.66 (d, J=8.4 Hz, 2H), 7.53 (d, J=8.4 Hz, 2H), 4.98 (q, J=6.4 Hz, 1H), 1.6 (s, 1H), 1.54 (d, J=6.0 Hz, 3H).

### Intermediate 11-3

### 1-(4-(1-Bromoethyl)phenyl)-1H-1,2,4-triazole

To a solution of 1-(4-(1H-1,2,4-triazol-1-yl)phenyl)ethanol 11-2 (100 mg, 93% purity, 0.49 mmol) in dichloromethane (2 mL) was added tribromophosphine (300 mg, 0.37 mmol) in dichloromethane (1 mL) dropwise at 0° C., the resulting mixture was stirred at 0° C. for 3 hours.

The reaction was concentrated under reduced pressure to give the crude product (540 mg, 71% purity from ¹H NMR, 72.6% yield) as white solids. The crude was used for next step without purification. LC-MS (ESI): R_{T}=1.65 min, mass calcd. for C₁₀H₁₀BrN₃ 251.0 m/z found 252.0 [M+H]⁺.

### Intermediate 11

### 5-((3R,7R)-9-(1-(4-(1H-1,2,4-triazol-1-yl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (130 mg, 100% yield, 0.34 mmol) and 1-(4-(1-bromoethyl)phenyl)-1H-1,2,4-triazole hydrobromide 11-3 (300 mg, 71% purity, 0.85 mmol) in 2-methyltetrahydrofuran (3 mL) were added sodium hydroxide (1.5 mL, 50% wt.) and N-benzyl-N, N-diethylethanaminium chloride (10 mg, 0.04 mmol) at 25° C. The resulting mixture was stirred at 50° C. for 2 hours. The reaction was poured into water (10 mL), extracted with acetate (10 mL) for three times. The combined organic layers were washed with brine (10 mL), dried over Na₂SO₄₍ₛ₎, and filtered. The filtrate was and concentrated in vacuum, which was purified by C18 chromatography (acetonitrile:water (+0.02% ammonium bicarbonate)=40-60%) to give the title compound (120 mg, 83.2% purity from LCMS, 53.1% yield) as white solids. LC-MS (ESI): R_{T}=3.352 min, mass calcd. for C₂₉H₂₇ClN₈O₂ 554.2 m/z found 555.1 [M+H]⁺.

### Compounds 11A and 11B

### 5-((3R,7R)-9-((R*)-1-(4-(1H-1,2,4-Triazol-1-yl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile (11A) and 5-((3R,7R)-9-((S*)-1-(4-(1H-1,2,4-triazol-1-yl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile (11B)

The racemate 5-((3R,7R)-9-(1-(4-(1H-1,2,4-triazol-1-yl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile 11 (117 mg, 83.2% purity, 0.18 mmol) was separated by chiral prep HPLC (separation condition: Column: Chiralpak IE 5 μm 20*250 mm; Mobile Phase: MeOH:DCM=90:10 at 25 mL/min; Temp: 30° C.; Wavelength: 230 nm) to give the title compounds 11A (34.6 mg, 98.6% purity, 35.0% yield, 100% stereopure) and 11B (39.5 mg, 97.5% purity, 39.6% yield, 99.1% stereopure) as white solids.

### 11A:

LC-MS (ESI): R_{T}=4.486 min, mass calcd. for C₂₉H₂₇ClN₈O₂ 554.2 m/z found 555.2 [M+H]⁺. Chiral analysis: (Column: Chiralpak IE 5 m 4.6*250 mm; Mobile Phase: MeOH:DCM=90:10 at 1 mL/min; Temp: 30° C.; Wavelength: 230 nm, R_{T}=11.006 min). ¹H NMR (400 MHz, DMSO-d₆) δ 9.31 (s, 1H), 8.24 (s, 1H) 8.18-8.09 (m, 1H), 7.91-7.79 (m, 4H), 7.61-7.50 (m, 2H), 5.99-5.76 (m, 1H), 5.55-5.15 (m, 1H), 4.65-4.37 (m, 2H), 4.22-4.07 (m, 1H), 3.87-3.72 (m, 1H), 3.13-2.87 (m, 2H), 2.54-2.51 (m, 1H), 1.67-1.50 (m, 3H), 1.24 (d, J=6.8 Hz, 3H), 1.21-1.05 (m, 3H).

### 11B:

LC-MS (ESI): R_{T}=4.146 min, mass calcd. for C₂₉H₂₇ClN₈O₂ 554.2 m/z found 555.3 [M+H]⁺. Chiral analysis: (Column: Chiralpak IE 5 m 4.6*250 mm; Mobile Phase: MeOH:DCM=90:10 at 1 mL/min; Temp: 30° C.; Wavelength: 230 nm, R_{T}=13.091 min). ¹H NMR (400 MHz, DMSO-d₆) δ 9.30 (s, 1H), 8.23 (s, 1H) 8.19-8.08 (m, 1H), 7.90-7.78 (m, 4H), 7.63-7.46 (m, 2H), 5.99-5.73 (m, 1H), 5.56-5.12 (m, 1H), 4.66-4.31 (m, 2H), 4.26-4.05 (m, 1H), 3.55-3.35 (m, 2H), 3.104-2.87 (m, 1H), 2.57-2.51 (m, 1H), 1.65-1.52 (m, 3H), 1.43 (d, J=6.4 Hz, 3H), 1.26-1.07 (m, 3H).

### Compounds 12A and 12B

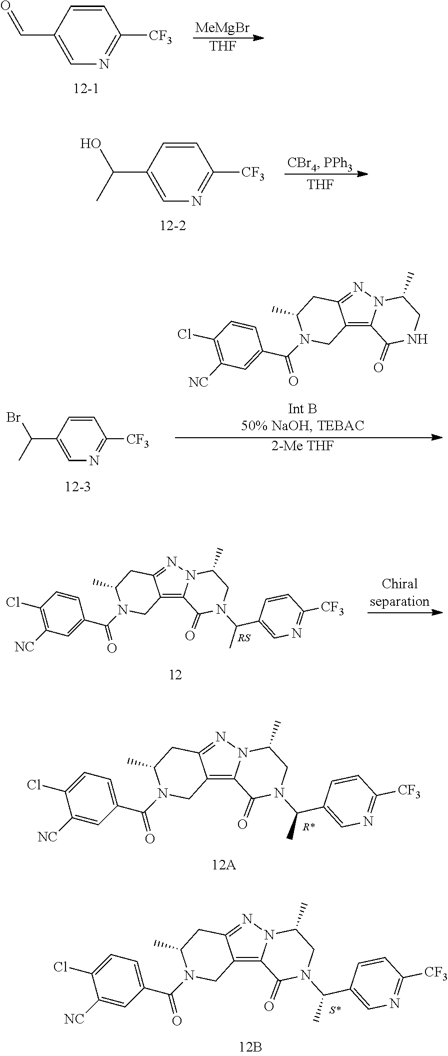

### Intermediate 12-2

### 1-(6-(Trifluoromethyl)pyridin-3-yl)ethanol

To a solution of 6-(trifluoromethyl)nicotinaldehyde 12-1 (900 mg, 5.14 mmol) in tetrahydrofuran (10 mL) was added dropwise 3 M methylmagnesium bromide in 2-methyltetrahydrofuran (2.7 mL, 8.10 mmol) at −70° C. for 2 hours. The reaction mixture was poured into ammonium chloride aqueous solution (40 mL) and extracted with ethyl acetate (20 mL) twice. The combined organic layers were washed with brine (20 mL), dried over Na₂SO₄₍ₛ₎ and filtered. The filtrate was concentrated to give the title compound (900 mg, 90% purity from ¹H NMR, 82% yield) as colorless oil. ¹H NMR (400 MHz, CDCl₃) δ 8.68 (br s, 1H), 7.93-7.91 (m, 1H), 7.67 (d, J=8.0 Hz, 1H), 5.07-5.02 (m, 1H), 2.64 (br s, 1H), 1.55 (d, J=6.4 Hz, 3H).

### Intermediate 12-3

### 5-(1-Bromoethyl)-2-(trifluoromethyl)pyridine

To a solution of 1-(6-(Trifluoromethyl)pyridin-3-yl)ethanol 12-2 (900 mg, 90% purity, 4.24 mmol) in tetrahydrofuran (15 mL) were added triphenylphosphine (2.0 g, 7.63 mmol) and perbromomethane (2.1 g, 6.33 mmol) at 0° C. After stirring at 25° C. for 0.5 hour, the mixture was filtered. The filtrate was concentrated under reduced pressure to give the residue, which was purified by silica gel column chromatography (petroleum ether:ethyl acetate=100:1) to give the title compound (1.0 g, 90% purity by ¹H NMR, 84% yield) as colorless oil. ¹H NMR (400 MHz, CDCl₃) δ 8.77 (br s, 1H), 7.98-7.95 (m, 1H), 7.68 (d, J=8.0, 1H), 5.24-5.18 (m, 1H), 2.80 (d, J=6.8 Hz, 3H).

### Compound 12

### 2-Chloro-5-((3R,7R)-3,7-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-2-carbonyl)benzonitrile

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (355 mg, 91% purity, 0.84 mmol) and 5-(1-bromoethyl)-2-(trifluoromethyl)pyridine 12-3 (500 mg, 95% purity, 1.87 mmol) in 2-methyltetrahydrofuran (2 mL) was added 50% wt. sodium hydroxide in water (2 mL) and N-benzyl-N,N-diethylethanaminium chloride (30 mg, 0.13 mmol) slowly at room temperature. After stirring at room temperature for 2 hours, the mixture was diluted with water (10 mL) and concentrated at room temperature under reduced pressure to remove the volatile. The remained aqueous layer was acidified with 1 M hydrochloride aqueous solution (20 mL) and extracted with ethyl acetate (50 mL) twice. The combined organic layers were washed with brine (30 mL), dried over Na₂SO₄₍ₛ₎ and filtered. The filtrate was concentrated and purified by C18 column (acetonitrile:water=55% to 75%) to give the crude compound. The crude compound was purified by silica gel column chromatography (dichloromethane:methanol=10:1) to give the title compound (330 mg, 98% purity from LCMS, 69.0% yield) as yellow solids. LC-MS (ESI): R_{T}=1.59 min, mass calcd. For C₂₇H₂₄ClF₃N₆O₂ 556.2, m/z found 557.2 [M+H]⁺.

### Compounds 12A and 12B

### 2-Chloro-5-((3R,7R)-3,7-dimethyl-10-oxo-9-((R*)-1-(6-(trifluoromethyl)pyridin-3-yl)ethyl)-1,2,3,4,7,8,9,10-octahydropyrido[4′,3′:3,4]pyrazolo[1,5-a]pyrazine-2-carbonyl)benzonitrile (12A) and 2-Chloro-5-((3R,7R)-3,7-dimethyl-10-oxo-9-((S*)-1-(6-(trifluoromethyl)pyridin-3-yl)ethyl)-1,2,3,4,7,8,9,10-octahydropyrido[4′,3′:3,4]pyrazolo[1,5-a]pyrazine-2-carbonyl)benzonitrile (12B)

The racemate of 2-chloro-5-((3R,7R)-3,7-dimethyl-10-oxo-9-(1-(6-(trifluoromethyl)pyridin-3-yl)ethyl)-1,2,3,4,7,8,9,10-octahydropyrido[4′,3′:3,4]pyrazolo[1,5-a]pyrazine-2-carbonyl)benzonitrile 12 (370 mg, 98% purity, 0.65 mmol) was separated by chiral Prep. HPLC (separation condition: Column: Chiralpak IC 5 m 30*250 mm, Mobile Phase: ACN:IPA=90:10 at 30 mL/min; Temp: 30° C.; Wavelength: 254 nm) to afford the compounds 12A (80 mg, 99.1% purity, 21.9% yield, 100% stereopure) as white solids and 12B (78 mg, 99.3% purity, 21.4% yield, 99.9% stereopure) as white solids.

### 12A:

LC-MS (ESI): R_{T}=3.306 min, mass calcd. for C₂₇H₂₄ClF₃N₆O₂ 556.2, m/z found 557.2 [M+H]⁺. Chiral analysis (Column: Superchiral IC 5 m 4.6*250 mm; Mobile Phase: ACN:IPA=90:10 at 1 mL/min; Temp: 30° C.; Wavelength: 254 nm, R_{T}=4.152 min). ¹H NMR (400 MHz, CDCl₃) δ 8.76 (s, 1H), 7.92-7.84 (m, 1H), 7.74-7.69 (m, 2H), 7.62-7.58 (m, 2H), 6.14 (br s, 1H), 5.77-5.40 (m, 1H), 4.76-4.25 (m, 3H), 3.69-3.66 (m, 1H), 3.13-2.93 (m, 2H), 2.69 (d, J=15.6 Hz, 1H), 1.67 (d, J=7.2 Hz, 3H), 1.36 (d, J=6.4 Hz, 3H), 1.29-1.28 (m, 3H). ¹⁹F NMR (376 MHz, CDCl₃) δ −67.94.

### 12B:

LC-MS (ESI): R_{T}=3.320 min, mass calcd. for C₂₇H₂₄ClF₃N₆O₂ 556.2, m/z found 557.2 [M+H]⁺. Chiral analysis (Column: Superchiral IC 5 m 4.6*250 mm; Mobile Phase: ACN:IPA=90:10 at 1 mL/min; Temp: 30° C.; Wavelength: 254 nm, R_{T}=5.124 min). ¹H NMR (400 MHz, CDCl₃) δ 8.73 (s, 1H), 7.89-7.81 (m, 1H), 7.75-7.68 (m, 2H), 7.63-7.58 (m, 2H), 6.13 (br s, 1H), 5.77-5.44 (m, 1H), 4.81-4.22 (m, 3H), 3.41-3.26 (m, 2H), 3.06 (br s, 1H), 2.70 (d, J=15.6 Hz, 1H), 1.68 (d, J=6.8 Hz, 3H), 1.58-1.56 (m, 3H), 1.33-1.22 (m, 3H). ¹⁹F NMR 6 (376 MHz, CDCl₃) δ −67.94.

### Compounds 13A and 13B

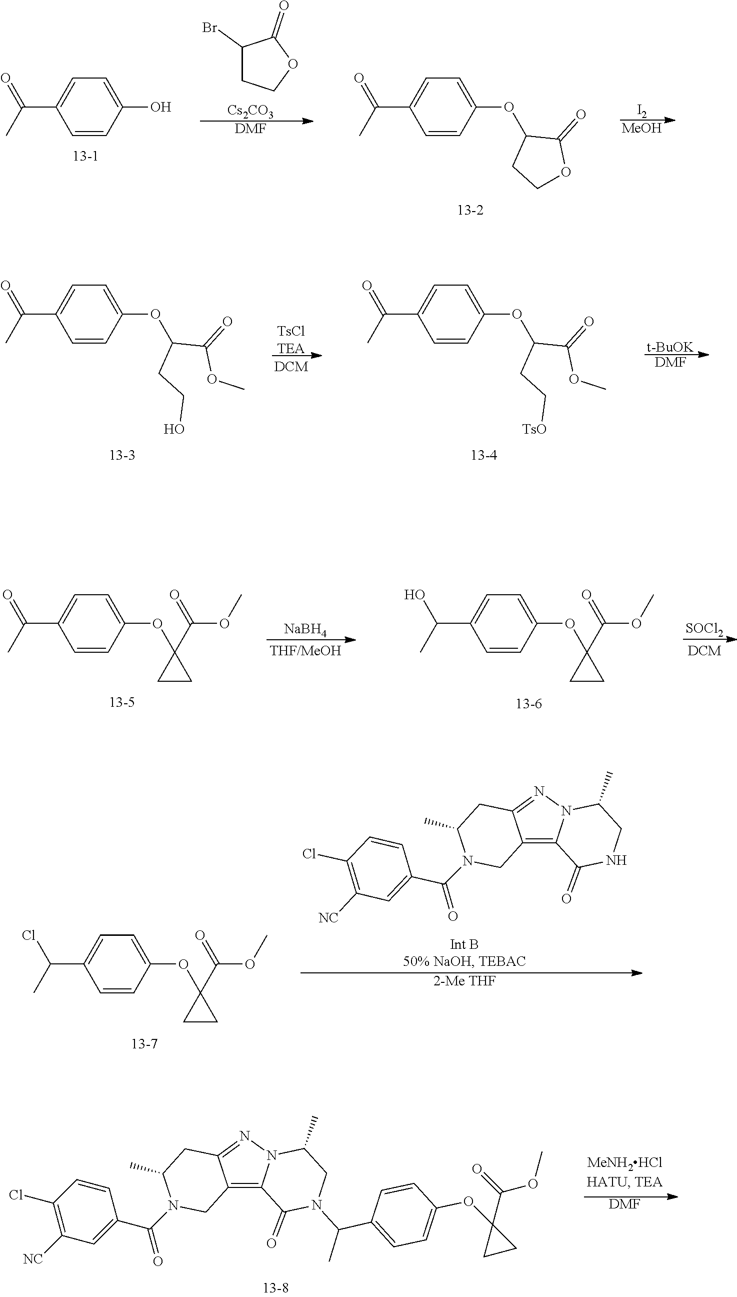

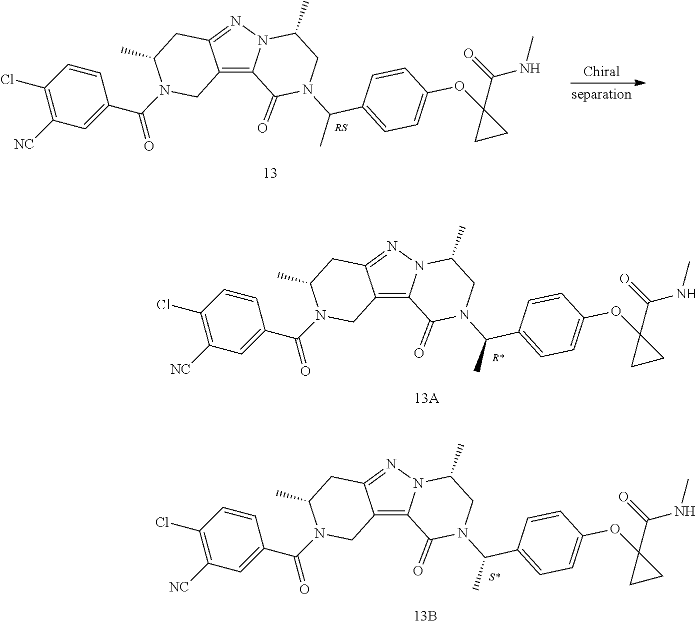 [SMILES: CNC(=O)C1(Oc2ccc(C(C)N3C[C@@H](C)n4nc5c(c4C3=O)CN(C(=O)c3ccc(Cl)c(C#N)c3)[C@H](C)C5)cc2)CC1 ; CNC(=O)C1(Oc2ccc([C@@H](C)N3C[C@@H](C)n4nc5c(c4C3=O)CN(C(=O)c3ccc(Cl)c(C#N)c3)[C@H](C)C5)cc2)CC1 ; CNC(=O)C1(Oc2ccc([C@H](C)N3C[C@@H](C)n4nc5c(c4C3=O)CN(C(=O)c3ccc(Cl)c(C#N)c3)[C@H](C)C5)cc2)CC1]

### Intermediate 13-2

### 3-(4-Acetylphenoxy)dihydrofuran-2(3H)-one

To the solution of 1-(4-hydroxyphenyl)ethanone 13-1 (10 g, 73.4 mmol) and 3-bromodihydrofuran-2(3H)-one (13 mL, 141 mmol) in N,N-dimethylformamide (200 mL) was added cesium carbonate (48 g, 147 mmol) at 0° C. After stirred at room temperature overnight, the mixture was diluted with water (500 mL), extracted with ethyl acetate (200 mL) for three times. The combined organic layers were concentrated and purified by silica gel column chromatography (petroleum ether:ethyl acetate=3:1 to 1:1) to give the title compound (14.3 g, 100% purity from LCMS, 88% yield) as white solids. LC-MS (ESI): R_{T}=1.42 min, mass calcd. for C₁₂H₁₂O₄ 220.1, m/z found 221.1 [M+H]⁺.

### Intermediate 13-3

### Methyl 2-(4-acetylphenoxy)-4-hydroxybutanoate

To the solution of 3-(4-acetylphenoxy)dihydrofuran-2(3H)-one 13-2 (13 g, 100% purity, 59.0 mmol) in methanol (600 mL) was added iodine (2 g, 7.88 mmol). After stirred at 80° C. for 5 hours, the solution was cooled down and extracted with saturated sodium sulfite solution at 0° C. The mixture was concentrated to removed methanol, diluted with water (100 mL) and extracted with ethyl acetate (100 mL) twice. The combined organic layers were dried over Na₂SO₄₍ₛ₎ and filtered. The filtrate was concentrated to give the title compound (13 g, 98% purity from LCMS, 86% yield) as colorless oil. LC-MS (ESI): R_{T}=1.20 min, mass calcd. for C₁₃H₁₆O₅ 252.1, m/z found 253.1 [M+H]⁺.

### Intermediate 13-4

### Methyl 2-(4-acetylphenoxy)-4-(tosyloxy)butanoate

To a solution of methyl 2-(4-acetylphenoxy)-4-hydroxybutanoate 13-3 (13 g, 98% purity, 50.5 mmol) in dichloromethane (300 mL) was added tosyl chloride (14 g, 73.4 mmol) and triethylamine (21 mL, 151 mmol) at 0° C. After stirred at room temperature for 15 hours, the mixture was concentrated and purified by silica gel column chromatography (petroleum ether:ethyl acetate=4:1) to give the title compound (16.5 g, 98% purity from LCMS, 79% yield) as colorless oil. LC-MS (ESI): R_{T}=1.62 min, mass calcd. for C₂₀H₂₂07S 406.1, m/z found 407.1 [M+H]⁺.

### Intermediate 13-5

### Methyl 1-(4-acetylphenoxy)cyclopropanecarboxylate

To the solution of methyl 2-(4-acetylphenoxy)-4-(tosyloxy)butanoate 13-4 (13.5 g, 98% purity, 32.6 mmol) in N,N-dimethylformamide (600 mL) was added potassium tert-butoxide (4.5 g, 40.1 mmol) at 0° C. After stirred for 1 hour, the solution was diluted with ethyl acetate (500 mL), washed with water (500 mL) twice and brine (400 mL), dried over Na₂SO₄₍ₛ₎ and filtered. The filtrate was concentrated and purified by silica gel column chromatography (petroleum ether:ethyl acetate=9:1) to give the title compound (3.7 g, 100% purity from LCMS, 49% yield) as white solids. LC-MS (ESI): R_{T}=1.49 min, mass calcd. for C₁₃H₁₄O₄ 234.1, m/z found 235.1 [M+H]⁺.

### Intermediate 13-6

### Methyl 1-(4-(1-hydroxyethyl)phenoxy)cyclopropanecarboxylate

To the solution of methyl 1-(4-acetylphenoxy)cyclopropanecarboxylate 13-5 (1 g, 100% purity, 4.27 mmol) in tetrahydrofuran (6 mL) and methanol (2 mL) was added sodium borohydride (160 mg, 4.23 mmol) at 0° C. After stirred at room temperature for 1 hours, the mixture was quenched with saturated ammonium chloride aqueous solution (20 mL), extracted with ethyl acetate (20 mL) twice. The combined organic layers were dried over Na₂SO₄₍ₛ₎ and filtered. The filtrate was concentrated to give the title compound (1.1 g, 90% purity from ¹H NMR, 98% yield) as colorless oil. ¹H NMR (400 MHz, CDCl₃) δ 7.28 (d, J=8.4 Hz, 2H), 6.89 (d, J=8.4 Hz, 2H), 4.85 (q, J=6.4 Hz, 1H), 3.73 (s, 3H), 1.62 (dd, J=8.4 and 4.8 Hz, 2H), 1.48 (d, J=6.4 Hz, 3H), 1.31 (dd, J=8.0 and 5.2 Hz, 2H).

### Intermediate 13-7

### Methyl 1-(4-(1-chloroethyl)phenoxy)cyclopropanecarboxylate

To the solution of methyl 1-(4-(1-hydroxyethyl)phenoxy)cyclopropanecarboxylate 13-6 (1.1 g, 90% purity, 4.19 mmol) in dichloromethane (10 mL) was added thionyl chloride (0.6 mL, 8.27 mmol). After stirred at 30° C. for 3 hours, the mixture was cooled down, quenched with saturated sodium bicarbonate aqueous solution (20 mL) and extracted with dichloromethane (20 mL) twice. The combined organic layers were dried over Na₂SO₄₍ₛ₎ and filtered. The filtrate was concentrated to give the title compound (1 g, 95% purity from ¹H NMR, 89% yield) as colorless oil. ¹H NMR (400 MHz, CDCl₃) δ 7.32 (d, J=8.8 Hz, 2H), 6.88 (d, J=8.8 Hz, 2H), 5.08 (q, J=6.8 Hz, 1H), 3.73 (s, 3H), 1.83 (d, J=6.8 Hz, 3H), 1.62 (dd, J=8.8 and 5.2 Hz, 2H), 1.31 (dd, J=8.0 and 4.8 Hz, 2H).

### Intermediate 13-8

### 1-(4-(1-((3R,7R)-2-(4-Chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)cyclopropanecarboxylic acid

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (600 mg, 100% purity, 1.56 mmol) and methyl 1-(4-(1-chloroethyl)phenoxy)cyclopropanecarboxylate 13-7 (1 g, 95% purity, 3.73 mmol) in 2-methyltetrahydrofuran (3 mL) were added 50% wt. sodium hydroxide aqueous solution (3 mL) and benzyltriethylammonium chloride (40 mg, 0.176 mmol). After stirred at 30° C. for 5 hours, the reaction mixture was acidized with 1 N hydrochloride to pH˜4, extracted with ethyl acetate (10 mL) for three times. The combined organic layers were dried over Na₂SO₄₍ₛ₎, filtered. The filtrate was concentrated and purified by C18 column (acetonitrile:water=50% to 70%) to give the title compound (330 mg, 88% purity from LCMS, 32% yield) as white solids. LC-MS (ESI): R_{T}=0.87 min, mass calcd. for C₃₁H₃₀ClN₅O₅ 587.2, m/z found 588.2 [M+H]⁺.

### Compound 13

### 1-(4-(1-((3R,7R)-2-(4-Chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)-N-methylcyclopropanecarboxamide

To a solution of 1-(4-(1-((3R,7R)-2-(4-chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)cyclopropanecarboxylic acid 13-8 (330 mg, 88% purity, 0.465 mmol) and methylamine hydrochloride (60 mg, 95% purity, 0.800 mmol) in N,N-dimethylformamide (4 mL) were added o-(7-azabenzotriazol-1-yl)-N,N,N′,N′-tetramethyluronium hexafluorophosphate (270 mg, 0.710 mmol) and triethylamine (0.2 mL, 1.44 mmol). After stirred at room temperature for 1 hour, the reaction mixture was diluted with ethyl acetate (20 mL), washed with water (10 mL), brine (10 mL), dried over Na₂SO₄₍ₛ₎ and filtered. The filtrate was concentrated and purified by C18 column (acetonitrile:water=50% to 70%) to give the title compound (160 mg, 89% purity from LCMS, 51% yield) as white solids. LC-MS (ESI): R_{T}=0.87 min, mass calcd. for C₃₂H₃₃ClN₆O₄ 600.2, m/z found 601.5 [M+H]⁺.

### Compounds 13A and 13B

### 1-(4-((R*)-1-((3R,7R)-2-(4-chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)-N-methylcyclopropanecarboxamide (13A) and 1-(4-((S*)-1-((3R,7R)-2-(4-chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)-N-methylcyclopropanecarboxamide (13B)

A racemate of 1-(4-(1-((3R,7R)-2-(4-chloro-3-cyanobenzoyl)-3,7-dimethyl-10-oxo-1,2,3,4,7,8-hexahydropyrido[4′,3′:3,4]pyrazolo[1,5-a]pyrazin-9(10H)-yl)ethyl)phenoxy)-N-methylcyclopropanecarboxamide 13 (130 mg, 89% purity, 0.192 mmol) was separated by chiral Prep. HPLC (separation condition: Column: Chiralcel OZ-H 3 m 4.6*150 mm; Mobile Phase: MeOH=100% at 1 mL/min; Col. Temp: 35° C.; Wavelength: 254 nm), then further purified to afford the title compound 13A (50 mg, 96.8% purity from LCMS, 42% yield, 99.1% stereopure) as white solids and 13B (40 mg, 97.1% purity from LCMS, 31% yield, 99.9% stereopure) as white solids.

### 13A:

LC-MS (ESI): R_{T}=3.544 min, mass calcd. for C₃₂H₃₃ClN₆O₄ 600.2, m/z found 601.3 [M+H]⁺. Chiral analysis (Column: Chiralcel OZ-H 5 m 4.6*250 mm; Mobile Phase: MeOH=100% at 1 mL/min; Temp: 35° C.; Wavelength: 254 nm), R_{T}=4.530 min). ¹H NMR (400 MHz, CDCl₃) δ 7.74 (s, 1H), 7.62-7.58 (m, 2H), 7.25-7.23 (m, 2H), 6.92 (d, J=8.8 Hz, 2H), 6.34-6.32 (m, 1H), 5.97-5.42 (m, 2H), 4.82-4.27 (m, 3H), 3.28 (d, J=6.0 Hz, 2H), 3.08 (br s, 1H), 2.82 (d, J=4.8 Hz, 3H), 2.69 (d, J=16.4 Hz, 1H), 1.65-1.60 (m, 2H), 1.57-1.52 (m, 6H), 1.28 (d, J=1.6 Hz, 3H), 1.14-1.11 (m, 2H).

### 13B:

LC-MS (ESI): R_{T}=3.490 min, mass calcd. for C₃₂H₃₃ClN₆O₄ 600.2, m/z found 601.3 [M+H]⁺. Chiral analysis (Column: Chiralcel OZ-H 5 m 4.6*250 mm; Mobile Phase: MeOH=100% at 1 mL/min; Temp: 35° C.; Wavelength: 254 nm), R_{T}=5.935 min). ¹H NMR (400 MHz, CDCl₃) δ 7.74 (s, 1H), 7.62-7.57 (m, 2H), 7.29-7.26 (m, 2H), 6.93 (d, J=8.4 Hz, 2H), 6.33-6.30 (m, 1H), 6.00 (br s, 1H), 5.74-5.39 (m, 1H), 4.76-4.37 (m, 3H), 3.61-3.58 (m, 1H), 3.12-2.99 (m, 2H), 2.82 (d, J=5.2 Hz, 3H), 2.70-2.66 (m, 1H), 1.63-1.60 (m, 2H), 1.55-1.53 (m, 3H), 1.30-1.25 (m, 6H), 1.14-1.11 (m, 2H).

### Compounds 14A and 14B

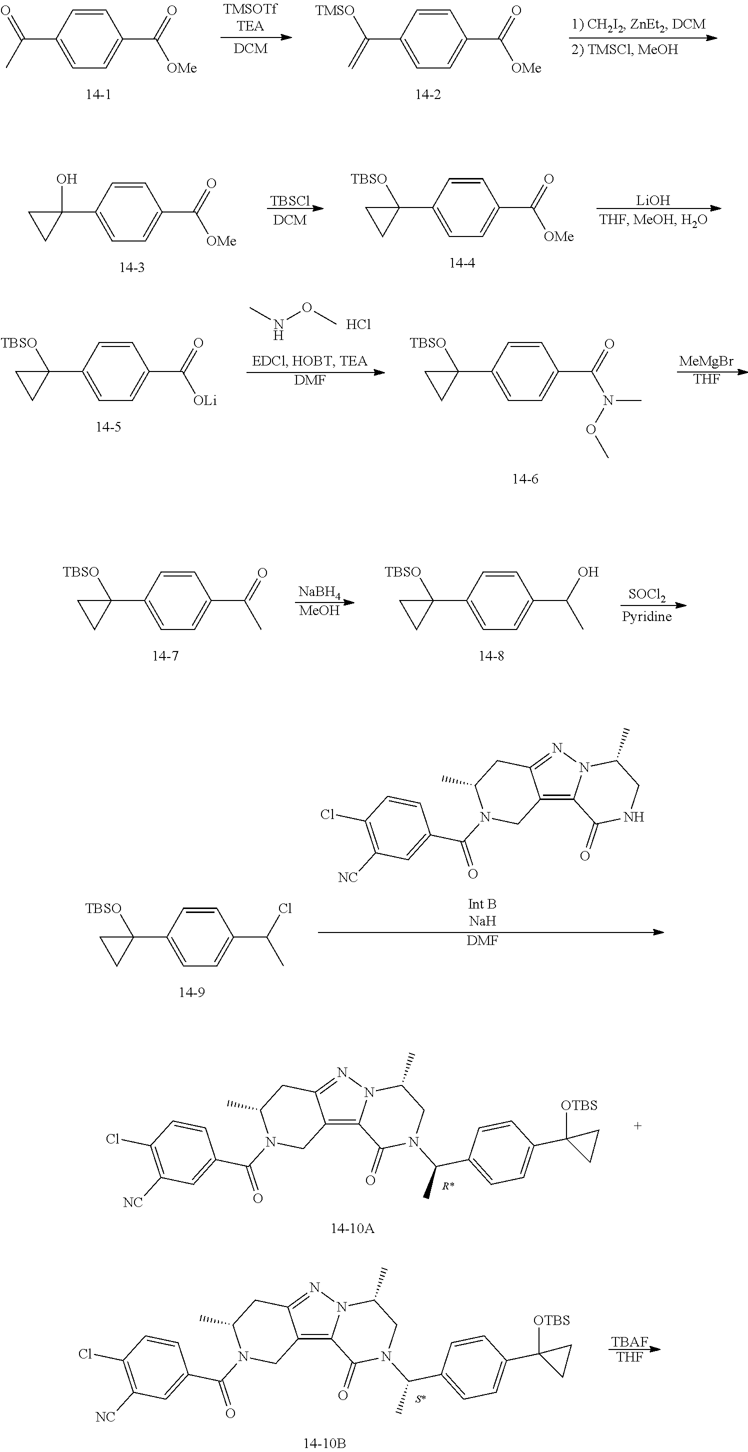

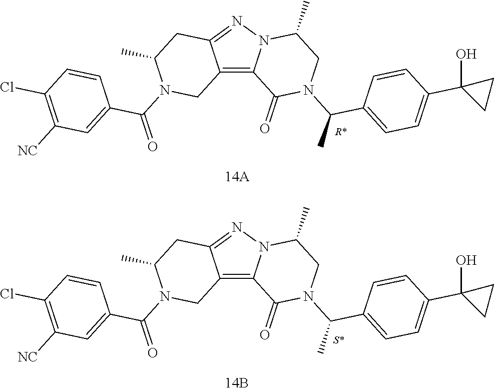 [SMILES: C[C@@H]1Cc2nn3c(c2CN1C(=O)c1ccc(Cl)c(C#N)c1)C(=O)N([C@H](C)c1ccc(C2(O)CC2)cc1)C[C@H]3C ; C[C@@H]1Cc2nn3c(c2CN1C(=O)c1ccc(Cl)c(C#N)c1)C(=O)N([C@@H](C)c1ccc(C2(O)CC2)cc1)C[C@H]3C]

### Intermediate 14-2

### Methyl 4-(1-((trimethylsilyl)oxy)vinyl)benzoate

To a solution of methyl 4-acetylbenzoate 14-1 (4.00 g, 22.4 mmol) in dichloromethane (60 mL) were added triethylamine (4.8 mL, 34.5 mmol) and trimethylsilyl trifluoromethanesulfonate (5.2 mL, 28.7 mmol) at 0° C. After stirred at 0° C. for 1 hour, the mixture was quenched with water (100 mL), extracted with dichloromethane (100 mL) twice. The combined organic layers were washed with saturated sodium bicarbonate aqueous solution (30 mL), brine (50 mL), dried over Na₂SO₄₍ₛ₎ and filtered. The filtrate was concentrated to give the title compound (6.00 g, 90% purity from ¹H NMR, 96% yield) which was used in next step without purification. LC-MS (ESI): R_{T}=3.123 min, mass calcd. for Cl₃H₁₈O₃Si 250.1, m/z found 251.1 [M+H]⁺. ¹H NMR (400 MHz, CDCl₃) δ 8.00-7.98 (m, 2H), 7.66-7.63 (m, 2H), 5.02 (d, J=2.0 Hz, 1H), 5.54 (d, J=2.0 Hz, 1H), 3.91 (s, 3H), 0.27 (s, 9H).

### Intermediate 14-3

### Methyl 4-(1-hydroxycyclopropyl)benzoate

To a solution of methyl 4-(1-((trimethylsilyl)oxy)vinyl)benzoate 14-2 (6.00 g, 90% purity, 21.6 mmol) and diiodomethane (9.20 g, 34.3 mmol) in dichloromethane (30 mL) was added 1.0 M diethylzinc in hexane (40 mL, 40.0 mmol) at 0° C. After stirred at 0° C. for 1 hour, the mixture was warmed up to room temperature and stirred for 3 days. Then the reaction was quenched with saturated ammonium chloride aqueous solution (50 mL) slowly, extracted with dichloromethane (50 mL) twice. The combined organic layers were washed with brine (50 mL), dried over Na₂SO₄₍ₛ₎, filtered and concentrated to give a residue, which was diluted with methanol (20 mL). Chlorotrimethylsilane (0.2 mL) was added at 0° C. After stirred at 0° C. for 0.5 hour, the mixture was concentrated to give a residue, which was purified by silica gel column chromatography (petroleum ether:ethyl acetate=100:1 to 8:1) to give the title compound (2.30 g, 90% purity from ¹H NMR, 50% yield) as yellow solids. LC-MS (ESI): R_{T}=0.74 min, mass calcd. for C₁₁H₁₂O₃ 192.1, m/z found 193.1 [M+H]⁺. ¹H NMR (400 MHz, CDCl₃) δ 7.99-7.96 (m, 2H), 7.33-7.30 (m, 2H), 3.90 (s, 3H), 2.51 (br s, 1H), 1.38-1.35 (m, 2H), 1.14-1.11 (m, 2H).

### Intermediate 14-4

### Methyl 4-(1-((tert-butyldimethylsilyl)oxy)cyclopropyl)benzoate

To a solution of methyl 4-(1-hydroxycyclopropyl)benzoate 14-3 (2.30 g, 90% purity, 10.8 mmol) in dichloromethane (20 mL) was added 1H-imidazole (1.5 g, 22.0 mmol) and tert-butylchlorodimethylsilane (2.00 g, 13.3 mmol) at 0° C. After stirred at room temperature overnight, the mixture was quenched with saturated ammonium chloride aqueous solution (20 mL) and extracted with dichloromethane (20 mL) twice. The combined organic layers were concentrated to give a residue, which was purified by silica gel column chromatography (petroleum ether:dichloromethane=10:1 to 4:1) to give the title compound (3.60 g, 90% purity from ¹H NMR, 98% yield) as yellow oil. ¹H NMR (400 MHz, CDCl₃) δ 7.98-7.95 (m, 2H), 7.36-7.33 (m, 2H), 3.90 (s, 3H), 1.28-1.25 (m, 2H), 1.07-1.04 (m, 2H), 0.90 (s, 9H), 0.01 (s, 6H).

### Intermediate 14-5

### Lithium 4-(1-((tert-butyldimethylsilyl)oxy)cyclopropyl)benzoate

To the solution of methyl 4-(1-((tert-butyldimethylsilyl)oxy)cyclopropyl)benzoate 14-4 (3.60 g, 90% purity, 10.6 mmol) in tetrahydrofuran (30 mL), methanol (15 mL) and water (7.5 mL) was added lithium hydroxide monohydrate (570 mg, 13.6 mmol) under nitrogen atmosphere. After stirred at 20° C. overnight, the reaction was concentrated to give the title compound (3.20 g, 90% purity from ¹H NMR, 91% yield) as white solids. ¹H NMR (400 MHz, CD₃OD) δ 7.87-7.85 (m, 2H), 7.32-7.29 (m, 2H), 1.17-1.14 (m, 2H), 1.03-1.00 (m, 2H), 0.87 (m, 9H), 0.03 (s, 6H).

### Intermediate 14-6

### 4-(1-((tert-Butyldimethylsilyl)oxy)cyclopropyl)-N-methoxy-N-methylbenzamide

To the solution of lithium 4-(1-((tert-butyldimethylsilyl)oxy)cyclopropyl)benzoate 14-5 (3.20 g, 90% purity, 9.65 mmol), N,O-dimethylhydroxylamine hydrochloride (2.10 g, 21.5 mmol), 1-hydroxybenzotriazole (2.60 g, 19.2 mmol) and triethylamine (6.7 mL, 48.1 mmol) in N,N-dimethylformamide (50 mL) was added N-(3-dimethylaminopropyl)-N′-ethylcarbodiimide hydrochloride (3.70 g, 19.3 mmol) at 0° C. After stirred at room temperature overnight, the mixture was quenched with saturated ammonium chloride aqueous solution (100 mL) slowly, extracted with ethyl acetate (100 mL) twice. The combined organic layers were washed with brine (50 mL), dried over Na₂SO₄₍ₛ₎, filtered and concentrated to give a residue, which was purified by silica gel column chromatography (petroleum ether:ethyl acetate:dichloromethane=50:1:1 to 16:1:1) to give the title compound (1.90 g, 90% purity from ¹H NMR, 53% yield) as yellow oil. LC-MS (ESI): R_{T}=1.76 min, mass calcd. for C₁₈H₂₉NO₃Si 335.2, m/z found 336.2 [M+H]⁺. ¹H NMR (400 MHz, CDCl₃) δ 7.64-7.62 (m, 2H), 7.34-7.31 (m, 2H), 3.55 (s, 3H), 3.35 (s, 3H), 1.25-1.22 (m, 2H), 1.05-1.02 (m, 2H), 0.89 (s, 9H), 0.01 (s, 6H).

### Intermediate 14-7

### 1-(4-(1-((tert-Butyldimethylsilyl)oxy)cyclopropyl)phenyl)ethanone

To a stirred solution of 4-(1-((tert-butyldimethylsilyl)oxy)cyclopropyl)-N-methoxy-N-methylbenzamide 14-6 (1.90 g, 90% purity, 5.10 mmol) in tetrahydrofuran (15 mL) cooled in 0° C. was added dropwise 1 M methyl magnesium bromide in tetrahydrofuran (9.5 mL, 9.5 mmol). After stirred at 0° C. for 1 hour, the reaction mixture was quenched with cold saturated ammonium chloride solution and extracted with ethyl acetate (100 mL) for three times. The combined organic layers were washed with brine (100 mL), dried over Na₂SO₄₍ₛ₎, filtered and concentrated under reduced pressure to give the title compound (1.60 g, 90% purity from ¹H NMR, 97% yield) as yellow solids. LC-MS (ESI): R_{T}=1.91 min, mass calcd. for C₁₇H₂₆O₂S₁ 290.2, m/z found 291.2 [M+H]⁺. ¹H NMR (400 MHz, CDCl₃) δ 7.91-7.88 (m, 2H), 7.38-7.35 (m, 2H), 2.59 (s, 3H), 1.30-1.27 (m, 2H), 1.08-1.05 (m, 2H), 0.91 (s, 9H), 0.02 (s, 6H).

### Intermediate 14-8

### 1-(4-(1-((tert-Butyldimethylsilyl)oxy)cyclopropyl)phenyl)ethanol

To a stirred solution of 1-(4-(1-((tert-butyldimethylsilyl)oxy)cyclopropyl)phenyl) ethanone 14-7 (1.60 g, 90% purity, 4.96 mmol) in methanol (20 mL) cooled in 0° C. was added sodium tetrahydroborate (400 mg, 10.6 mmol). After stirred at 0° C. for 1 hour, the reaction mixture was concentrated under reduced pressure to give a residue, which was purified by silica gel column chromatography (petroleum ether:ethyl acetate=10:1 to 8:1) to give the title compound (1.60 g, 90% purity from ¹H NMR, 99% yield) as yellow oil. LC-MS (ESI): R_{T}=1.75 min, mass calcd. for C₁₇H₂₈02Si 292.2, m/z found 275.2 [M−H₂O+H]⁺. ¹H NMR (400 MHz, CDCl₃) δ 7.32-7.28 (m, 4H), 4.89 (q, J=6.4 Hz, 1H), 2.04 (br s, 1H), 1.49 (d, J=6.4 Hz, 3H), 1.19-1.16 (m, 2H), 1.00-0.97 (m, 2H), 0.88 (s, 9H), −0.01 (s, 6H).

### Intermediate 14-9

### tert-Butyl(1-(4-(1-chloroethyl)phenyl)cyclopropoxy)dimethylsilane

To a solution of 1-(4-(1-((tert-butyldimethylsilyl)oxy)cyclopropyl)phenyl)ethanol 14-8 (1.60 g, 90% purity, 4.92 mmol) in dichloromethane (30 mL) were added pyridine (1.6 mL, 19.9 mmol) and sulfurous dichloride (0.4 mL, 5.51 mmol) at 0° C. After stirred at 0° C. for 0.5 hour, the mixture was purified quickly by a short silica gel column chromatography (petroleum ether:dichloromethane=4:1) to give the title compound (1.30 g, 90% purity from ¹H NMR, 76% yield) as yellow oil. ¹H NMR (400 MHz, CDCl₃) δ 7.36-7.33 (m, 2H), 7.30-7.27 (m, 2H), 5.09 (q, J=6.8 Hz, 1H), 1.85 (d, J=6.8 Hz, 3H), 1.21-1.18 (m, 2H), 1.01-0.98 (m, 2H), 0.88 (s, 9H), 0.00 (s, 6H).

### Intermediates 14-10A and 14-10B

### 5-((3R,7R)-9-((R*)-1-(4-(1-((tert-Butyldimethylsilyl)oxy)cyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile (14-10A) and 5-((3R,7R)-9-((S*)-1-(4-(1-((tert-Butyldimethylsilyl)oxy)cyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile (14-10B)

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (500 mg, 90% purity, 1.17 mmol) in N,N-dimethylformamide (5 mL) was added 60% wt. sodium hydride in mineral oil (145 mg, 3.63 mmol). After the mixture was stirred at 0° C. for 0.5 hour, a solution of tert-butyl(1-(4-(1-chloroethyl)phenyl)cyclopropoxy) dimethylsilane 14-9 (1.30 g, 90% purity, 3.76 mmol) in N,N-dimethyl formamide (3 mL) was added. After stirred at room temperature overnight, the mixture was poured into saturated ammonium chloride aqueous solution (20 mL) and extracted with ethyl acetate (20 mL) twice. The combined organic layer were concentrated to give a residue, which was purified by a short silica gel column chromatography (petroleum ether:tetrahydrofuran=10:1 to 1:1) and C18 column (acetonitrile:water (0.1% ammonium bicarbonate)=30%-95%) to give the title compounds 14-10A (130 mg, 90% purity from ¹H NMR, 15% yield, 99.9% stereopure) as yellow solids and the 14-10B (110 mg, 90% purity from ¹H NMR, 13% yield, 98.6% stereopure) as yellow solids.

### 14-10A:

LC-MS (ESI): R_{T}=1.90 min, mass calcd. for C₃₆H₄₄ClN₅O₃Si 657.3, m/z found 658.3 [M+H]⁺. Chiral analysis (Column: Chiralpak IA 5 μm 4.6*250 mm; Mobile Phase: Hex:EtOH=60:40 at 0.5 mL/min; Col. Temp: 30° C.; Wavelength: 254 nm, R_{T}=7.728 min). ¹H NMR (400 MHz, CDCl₃) δ 7.76 (s, 1H), 7.62-7.58 (m, 2H), 7.33-7.24 (m, 4H), 6.19-5.91 (m, 1H), 5.81-5.30 (m, 1H), 4.93-4.10 (m, 3H), 3.30-3.19 (m, 2H), 3.08-2.86 (m, 1H), 2.77-2.62 (m, 1H), 1.57 (s, 3H), 1.51 (d, J=6.4 Hz, 3H), 1.29-1.26 (m, 3H), 1.21-1.18 (m, 2H), 0.99-0.96 (m, 2H), 0.86 (s, 9H), −0.02 (s, 6H).

### 14-10B:

LC-MS (ESI): R_{T}=1.92 min, mass calcd. for C₃₆H₄₄ClN₅O₃Si 657.3, m/z found 658.3 [M+H]⁺. Chiral analysis (Column: Chiralpak IA 5 m 4.6*250 mm; Mobile Phase: Hex:EtOH=60:40 at 0.5 mL/min; Col. Temp: 30° C.; Wavelength: 254 nm, R_{T}=10.858 min). ¹H NMR (400 MHz, CDCl₃) δ 7.74 (s, 1H), 7.62-7.57 (m, 2H), 7.34-7.30 (m, 4H), 6.18-5.95 (m, 1H), 5.86-5.33 (m, 1H), 4.87-4.20 (m, 3H), 3.67-3.54 (m, 1H), 3.17-2.97 (m, 2H), 2.70-2.62 (m, 1H), 1.56 (s, 3H), 1.30-1.26 (m, 3H), 1.23-1.16 (m, 5H), 1.02-0.94 (m, 2H), 0.87 (s, 9H), −0.01-−0.03 (m, 6H).

### Compound 14A

### 2-Chloro-5-((3R,7R)-9-((R*)-1-(4-(1-hydroxycyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile

To a solution of 5-((3R,7R)-9-((R*)-1-(4-(1-((tert-butyldimethylsilyl)oxy)cyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile 14-10A (130 mg, 90% purity, 0.178 mmol) in tetrahydrofuran (2 mL) was added 1 M tetrabutylammonium fluoride in (0.5 mL, 0.5 mmol) at 0° C. After stirred at 0° C. for 2 hours, the mixture was quenched with saturated ammonium chloride aqueous solution (5 mL) and extracted with dichloromethane (10 mL) twice. The combined organic layer were concentrated to give a residue, which was purified by a short silica gel column chromatography (petroleum ether:acetone=4:1 to 2:1) and Prep. HPLC (Column: Xbridge C18 (5 m 19*150 mm), Mobile Phase A: water (0.1% ammonium acetate), Mobile Phase B: acetonitrile, UV: 214 nm, Flow rate: 15 mL/min, Gradient: 30-60% (% B)) to give the title compound (32.8 mg, 99.8% purity, 34% yield) as white solids. LC-MS (ESI): R_{T}=3.825 min, mass calcd. for C₃₀H₃₀ClN₅O₃ 543.2, m/z found 544.3 [M+H]⁺. ¹H NMR (400 MHz, DMSO-d₆) δ 8.19-8.07 (m, 1H), 7.87-7.80 (m, 2H), 7.28-7.15 (m, 4H), 5.89-5.71 (m, 2H), 5.50-5.18 (m, 1H), 4.57-4.48 (m, 1H), 4.40-4.27 (m, 1H), 4.20-4.08 (m, 1H), 3.47-3.38 (m, 1H), 3.02-2.89 (m, 1H), 2.66-2.52 (m, 2H), 1.59-1.48 (m, 3H), 1.41 (d, J=6.4 Hz, 3H), 1.24-1.07 (m, 5H), 0.96-0.86 (m, 2H).

### Compound 14B

### 2-Chloro-5-((3R,7R)-9-((S*)-1-(4-(1-hydroxycyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile

To a solution of 5-((3R,7R)-9-((S*)-1-(4-(1-((tert-butyldimethylsilyl)oxy)cyclopropyl)phenyl)ethyl)-3,7-dimethyl-10-oxo-1,2,3,4,7,8,9,10-octahydropyrido[4′,3′:3,4]pyrazolo[1,5-a]pyrazine-2-carbonyl)-2-chlorobenzonitrile 14-10B (110 mg, 90% purity, 0.150 mmol) in tetrahydrofuran (2 mL) was added 1 M tetrabutylammonium fluoride in (0.5 mL, 0.5 mmol) at 0° C. After stirred at 0° C. for 2 hours, the mixture was quenched with saturated ammonium chloride aqueous solution (5 mL) and extracted with dichloromethane (10 mL) twice. The combined organic layer were concentrated to give a residue, which was purified by a short silica gel column chromatography (petroleum ether:acetone=4:1 to 2:1) and Prep. HPLC (Column: Xbridge C18 (5 m 19*150 mm), Mobile Phase A: water (0.1% ammonium acetate), Mobile Phase B: acetonitrile, UV: 214 nm, Flow rate: 15 mL/min, Gradient: 30-70% (% B)) to give the title compound (33.1 mg, 99.6% purity, 40% yield) as white solids. LC-MS (ESI): R_{T}=3.773 min, mass calcd. for C₃₀H₃₀ClN₅O₃ 543.2, m/z found 544.3 [M+H]⁺. ¹H NMR (400 MHz, DMSO-d₆) δ 8.20-8.08 (m, 1H), 7.87-7.80 (m, 2H), 7.29-7.17 (m, 4H), 5.09-5.73 (m, 2H), 5.49-5.22 (m, 1H), 4.62-4.41 (m, 2H), 4.21-4.06 (m, 1H), 3.79-3.66 (m, 1H), 3.06-2.88 (m, 2H), 2.52-2.51 (m, 1H), 1.60-1.42 (m, 3H), 1.22-1.08 (m, 8H), 0.96-0.89 (m, 2H).

### Compounds 15A and 15B

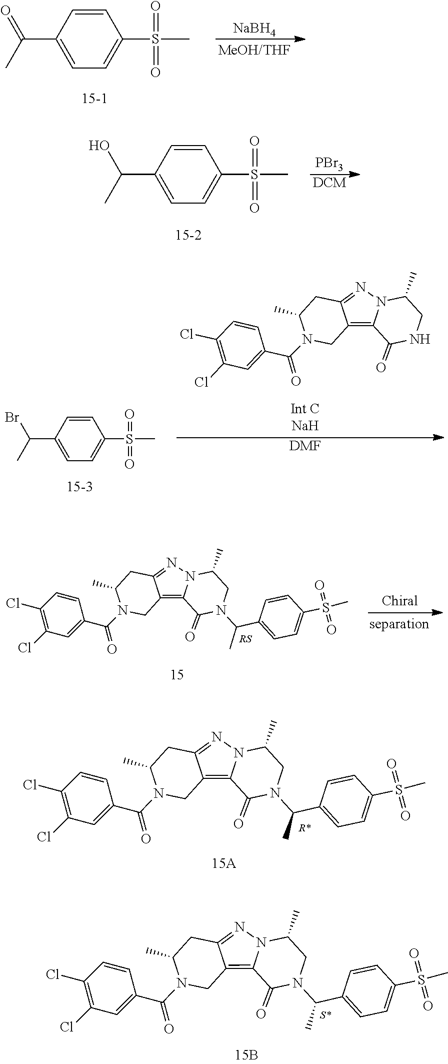

### Intermediate 15-2

### 1-(4-(Methylsulfonyl)phenyl)ethan-1-ol

To a solution of 1-(4-(methylsulfonyl)phenyl)ethenone 15-1 (1.0 g, 5.04 mmol) in tetrahydrofuran (5 mL) and methanol (10 mL) was added sodium tetrahydroborate (95 mg, 2.51 mmol) at 0° C. After stirred at 0° C. for 0.5 hour, the mixture was poured into water (30 mL) and quenched with 0.1 M hydrochloride aqueous solution (3 mL) and extracted with ethyl acetate (20 mL) twice. The combined organic layers were washed with brine (30 mL), dried over anhydrous Na₂SO₄₍ₛ₎ and filtered. The filtrated was concentrated under reduced pressure to give a residue, which was purified by silica gel column (petroleum ether:ethyl acetate=5:1 to 1:1) to give the title compound (900 mg, 90% purity from ¹H NMR, 80% yield) as white solids. ¹H NMR (400 MHz, CDCl₃) δ 7.92 (d, J=8.4 Hz, 2H), 7.58 (d, J=8.4 Hz, 2H), 5.01 (q, J=6.4 Hz, 1H), 3.05 (s, 3H), 2.01 (br s, 1H), 1.52 (d, J=6.4 Hz, 3H).

### Intermediate 15-3

### 1-(1-bromoethyl)-4-(methylsulfonyl)benzene

To a solution of 1-(4-(methylsulfonyl)phenyl)ethan-1-ol 15-2 (200 mg, 90% purity, 0.899 mmol) in dichloromethane (4 mL) was added dropwise tribromophosphane (729 mg, 2.693 mmol) in dichloromethane (1 mL) at 0° C. After stirred at 0° C. for 0.5 hours, the reaction mixture was quenched with saturated sodium bicarbonate aqueous solution (20 mL) and extracted with dichloromethane (20 mL) twice. The combined organic layers were washed with brine (30 mL), dried over Na₂SO₄₍ₛ₎, filtered and concentrated under reduced pressure to give the title compound (80 mg, 80% purity from ¹H NMR, 27% yield) as colorless oil. ¹H NMR (400 MHz, CDCl₃) δ 7.92 (d, J=8.4 Hz, 2H), 7.62 (d, J=8.4 Hz, 2H), 5.09 (q, J=7.2 Hz, 1H), 3.06 (s, 3H), 2.05 (d, J=7.2 Hz, 3H).

### Compound 15

### (3R,7R)-2-(3,4-dichlorobenzoyl)-3,7-dimethyl-9-(1-(4-(methylsulfonyl)phenyl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (253 mg, 100% purity, 0.643 mmol) in N,N-dimethylformamide (9 mL) was added 60% wt. sodium hydride in mineral oil (52 mg, 1.30 mmol). After stirred at 0° C. for 0.5 hour, 1-(1-bromoethyl)-4-(methylsulfonyl)benzene 15-3 (317 mg, 80% purity, 0.964 mmol) was added into the mixture. After stirred at 0° C. for 1 hour, the reaction mixture was quenched with water (30 mL) and extracted with ethyl acetate (25 mL) for three times. The combined organic layers were washed with water (30 mL) for three times, then washed with brine (30 mL), dried over Na₂SO₄₍ₛ₎ and filtered. The filtrate was concentrated and purified by C18 column (acetonitrile:water=5% to 100%) to give the title compound (210 mg, 100% purity, 57% yield) as white solids. LC-MS (ESI): R_{T}=1.61 min, mass calcd. for C₂₇H₂₈Cl₂N₆O₃ 574.1, m/z found 575.1 [M+H]⁺.

### Compounds 15A and 15B

### (3R,7R)-2-(3,4-dichlorobenzoyl)-3,7-dimethyl-9-((R*)-1-(4-(methylsulfonyl)phenyl)ethyl)-1,2,3,4,8,9-hexahydropyrido[4′,3′:3,4]pyrazolo[1,5-a]pyrazin-10(7H)-one (15A) and (3R,7R)-2-(3,4-dichlorobenzoyl)-3,7-dimethyl-9-((S*)-1-(4-(methylsulfonyl)phenyl)ethyl)-1,2,3,4,8,9-hexahydropyrido[4′,3′:3,4]pyrazolo[1,5-a]pyrazin-10(7H)-one (15B)

A racemic mixture of (3R,7R)-2-(3,4-dichlorobenzoyl)-3,7-dimethyl-9-(1-(4-(methyl sulfonyl)phenyl)ethyl)-1,2,3,4,8,9-hexahydropyrido[4′,3′:3,4]pyrazolo[1,5-a]pyrazin-10(7H)-one 15 (210 mg, 100% purity, 0.434 mmol) was separated by Chiral Prep-HPLC (Column: Chiralpak IE 5 μm 20 mm*250 mm; Mobile Phase: ACN:IPA=70:30 at 15 mL/min; Col. Temp: 30° C.; Wavelength: 254 nm) to afford the title compounds 15A (92 mg, 99% purity from LCMS, 37% yield, 100% steropure) as white solids and 15B (51 mg, 99.8% purity from LCMS, 20% yield, 99.9% steropure) as white solids.

### 15A:

LC-MS (ESI): R_{T}=3.438 min, mass calcd. for C₂₇H₂₈Cl₂N₆O₃ 574.1 m/z found 575.2 [M+H]⁺. Chiral analysis (Column: Chiralpak IE 5 μm 4.6*250 mm; Mobile Phase: ACN:IPA=70:30 at 1 mL/min; Temp: 30° C.; Wavelength: 254 nm, R_{T}=6.414 min). ¹H NMR (400 MHz, CDCl₃) δ 7.94 (d, J=8.4 Hz, 2H), 7.62-7.47 (m, 4H), 7.28-7.27 (m, 1H), 6.11 (br s, 1H), 5.81-5.19 (m, 1H), 5.01-4.15 (m, 3H), 3.39-3.20 (m, 2H), 3.13-2.96 (m, 4H), 2.71-2.66 (m, 1H), 1.64 (m, 3H), 1.55 (d, J=6.4 Hz, 3H), 1.27 (m, 3H).

### 15B:

LC-MS (ESI): R_{T}=3.494 min, mass calcd. for C₂₇H₂₈Cl₂N₆O₃ 574.1 m/z found 575.2 [M+H]⁺. Chiral analysis (Column: Chiralpak IE 5 μm 4.6*250 mm; Mobile Phase: ACN:IPA=70:50 at 1 mL/min; Temp: 30° C.; Wavelength: 254 nm, R_{T}=7.961 min). ¹H NMR (400 MHz, CDCl₃) δ 7.94 (d, J=8.4 Hz, 2H), 7.59-7.48 (m, 4H), 7.30-7.27 (m, 1H), 6.12 (br s, 1H), 5.80-5.13 (m, 1H), 4.98-4.53 (m, 1H), 4.42-4.19 (m, 2H), 3.38-3.22 (m, 2H), 3.13-3.00 (m, 4H), 2.74-2.61 (m, 1H), 1.64 (d, J=6.8 Hz, 3H), 1.55 (d, J=6.4 Hz, 3H), 1.26 (m, 3H).

### Compounds 16A and 16B

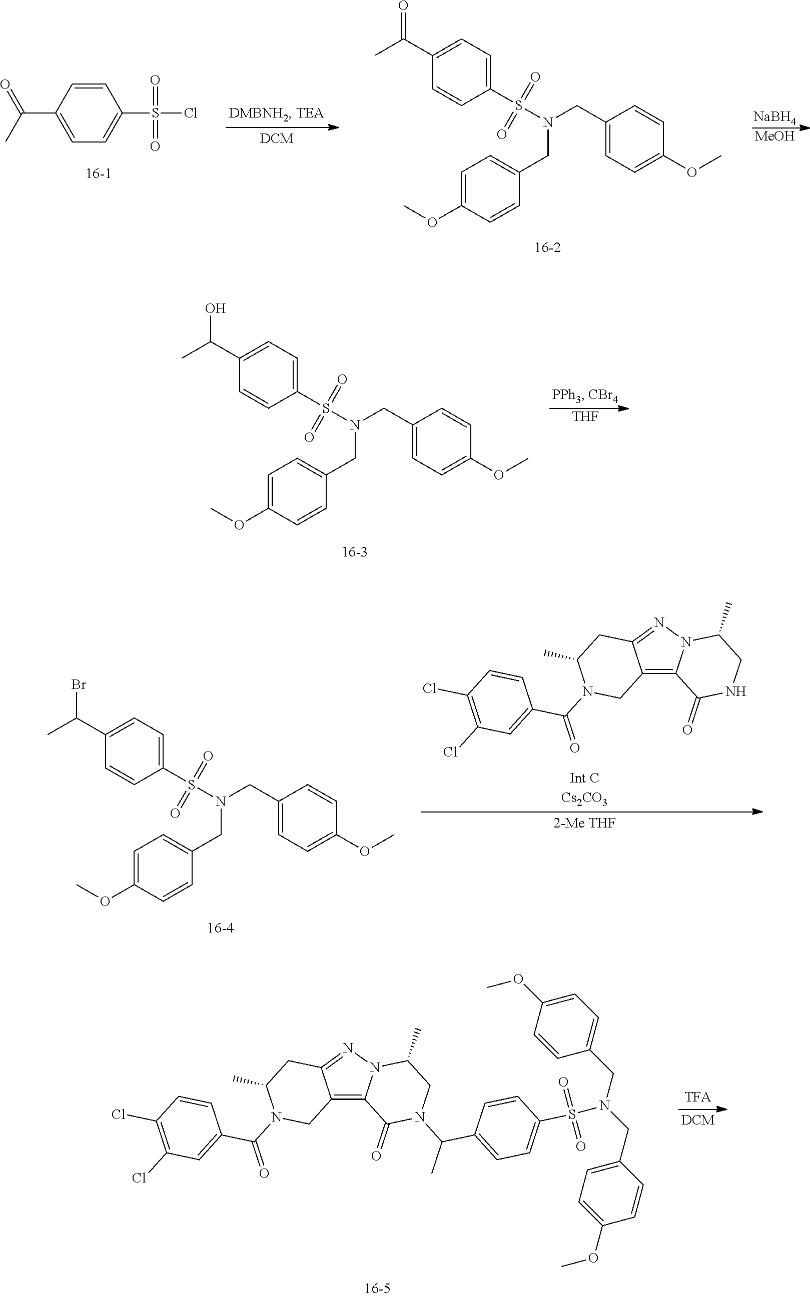

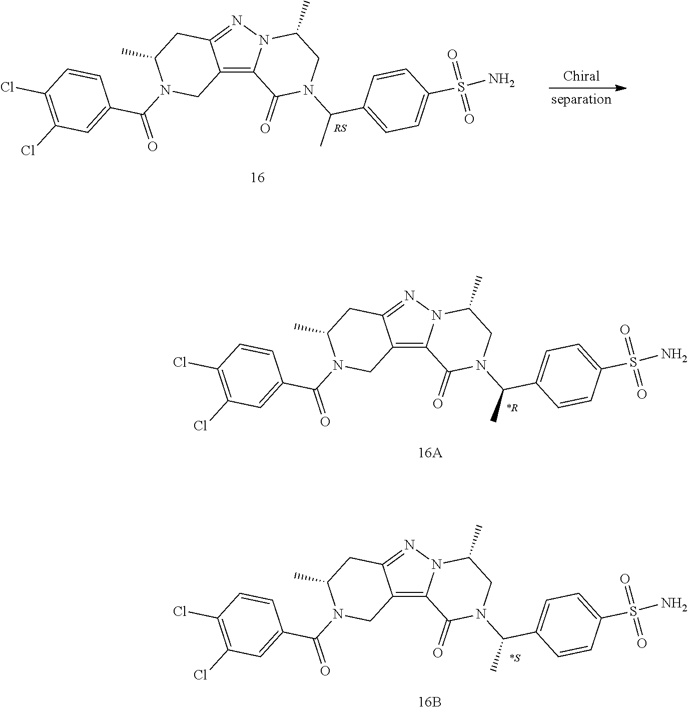 [SMILES: CC(c1ccc(S(N)(=O)=O)cc1)N1C[C@@H](C)n2nc3c(c2C1=O)CN(C(=O)c1ccc(Cl)c(Cl)c1)[C@H](C)C3 ; C[C@@H]1Cc2nn3c(c2CN1C(=O)c1ccc(Cl)c(Cl)c1)C(=O)N([C@H](C)c1ccc(S(N)(=O)=O)cc1)C[C@H]3C ; C[C@@H]1Cc2nn3c(c2CN1C(=O)c1ccc(Cl)c(Cl)c1)C(=O)N([C@@H](C)c1ccc(S(N)(=O)=O)cc1)C[C@H]3C]

### Intermediate 16-2

### 4-Acetyl-N,N-bis(4-methoxybenzyl)benzenesulfonamide

To a solution of 4-acetylbenzene-1-sulfonyl chloride 16-1 (6.70 g, 29.6 mmol) in dichloromethane (50 mL) were added triethylamine (6 mL, 43.1 mmol) and N-(2-methoxybenzyl)-1-(4-methoxyphenyl)methanamine (8.30 g, 32.3 mmol) at 0° C. under nitrogen atmosphere. After stirred at room temperature for 3 hours, the reaction mixture was added saturated sodium bicarbonate aqueous solution (100 mL) and extracted with dichloromethane (100 mL) for three times. The combined organic layers were washed with brine (100 mL), dried over Na₂SO₄₍ₛ₎ and concentrated to give the title compound (13.0 g, 80% purity from ¹H NMR, 80% yield) as yellow solids. ¹H NMR (400 MHz, CDCl₃) δ 8.05-7.88 (m, 4H), 6.99-6.96 (m, 4H), 6.77-6.75 (m, 4H), 4.27 (s, 4H), 3.78 (s, 6H), 2.66 (s, 3H).

### Intermediate 16-3

### 4-(1-Hydroxyethyl)-N,N-bis(4-methoxybenzyl)benzenesulfonamide

To a solution of 4-acetyl-N,N-bis(4-methoxybenzyl)benzenesulfonamide 16-2 (13.0 g, 80% purity, 23.7 mmol) in methanol (200 mL) was added sodium borohydride (7.00 g, 185 mmol) at 0° C. After stirred at 20° C. for 3 hours, the mixture was quenched with saturated ammonium chloride aqueous solution (200 mL) and extracted with dichloromethane (200 mL) for three times. The combined organic layers were washed with brine (100 mL), dried over Na₂SO₄₍ₛ₎ and concentrated to give the title compound (11.0 g, 76% purity from LCMS, 80% yield) as yellow solids. LC-MS (ESI): R_{T}=1.27 min, mass calcd. for C₂₄H₂₇NO₅S 441.2, m/z found 442.1.

### Intermediate 16-4

### 4-(1-Bromoethyl)-N,N-bis(4-methoxybenzyl)benzenesulfonamide

To a solution of 4-(1-hydroxyethyl)-N,N-bis(4-methoxybenzyl)benzenesulfonamide 16-3 (700 mg, 76% purity, 1.21 mmol) in tetrahydrofuran (12 mL) was added triphenylphosphine (550 mg, 2.10 mmol) and perbromomethane (550 mg, 1.66 mmol) at 0° C. After stirred at 25° C. for 2 hours, the reaction was concentrated to get a residue, which was purified by column chromatography on silica gel (petroleum ether:ethyl acetate=20:1 to 5:1) to give the desired compound (500 mg, 90% purity from ¹H NMR, 74% yield) as white solids. ¹H NMR (300 MHz, CDCl₃) δ 7.84-7.57 (m, 4H), 7.00-6.79 (m, 8H), 5.64-5.58 (m, 1H), 4.24-4.21 (m, 4H), 3.73 (s, 6H), 2.05-1.38 (m, 3H).

### Intermediate 16-5

### 4-(1-((3R,7R)-2-(3,4-Dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)-N,N-bis(4-methoxybenzyl)benzenesulfonamide

To a mixture of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (100 mg, 90% purity, 0.229 mmol) and 4-(1-bromoethyl)-N,N-bis(4-methoxybenzyl)benzenesulfonamide 16-4 (300 mg, 90% purity, 0.535 mmol) in N,N-dimethylformamide (2 mL) was added cesium carbonate (400 mg, 1.23 mmol). After stirred at 40° C. for 3 hours, the mixture was diluted with water (50 mL) and extracted with ethyl acetate (50 mL) twice. The combined organic layers were washed with brine (30 mL), dried over Na₂SO₄₍ₛ₎ and filtered. The filtrate was concentrated and purified by column chromatography on silica gel (petroleum ether:ethyl acetate=20:1 to 5:1) to give the desired compound (140 mg, 79% purity from LCMS, 60% yield) as white solids.

LC-MS (ESI): R_{T}=1.88 min, mass calcd. for C₄₂H₄₃Cl₂N₅O₆S 815.2, m/z found 833.1 [M+NH₄]⁺.

### Compound 16

### 4-(1-((3R,7R)-2-(3,4-Dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzenesulfonamide

To a solution of 4-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)-N,N-bis(4-methoxybenzyl)benzenesulfonamide 16-5 (220 mg, 79% purity, 0.214 mmol) in dichlormethane (1.5 ml) at 0° C. was added trifluoroacetic acid (0.5 ml) dropwise. After stirred at 35° C. for 3 hours, the reaction mixture was concentrated to give a residue, which was purified by C18 column (acetonitrile:water (0.1% ammonium bicarbonate)=5%-95%) to give the title compound (70 mg, 84% purity from LCMS, 48% yield) as white solids. LC-MS (ESI): R_{T}=1.57 min, mass calcd. for C₂₆H₂₇Cl₂N₅O₄S 575.1, m/z found 576.1 [M+H]⁺.

### Compounds 16A and 16B

### 4-((R*)-1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzenesulfonamide (16A) and 4-((S*)-1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzenesulfonamide (16B)

A racemate of 4-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)benzenesulfonamide 16 (70 mg, 84% purity, 0.102 mmol) was separated by Chiral Prep. HPLC (Column: Chiralpak IE 10 μm 30 mm*250 mm; Mobile Phase: MeOH:EtOH=50:50 at 25 mL/min; Col. Temp: 30° C.; Wavelength: 254 nm) to afford the title compounds 16A (25 mg, 99.9% purity from LCMS, 42.3% yield, 100% stereopure) as white solids and 16B (28 mg, 99.5% purity from LCMS, 47.2% yield, 99.9% stereopure) as white solids.

### 16A:

LC-MS (ESI): R_{T}=3.103 min, mass calcd. for C₂₆H₂₇Cl₂N₅O₄S 575.1, m/z found 576.1 [M+H]⁺. Chiral analysis: Column: Chiralpak IE 5 μm, 4.6*250 mm; Mobile Phase: MeOH:EtOH=50:50 at 1 mL/min; Temp 30° C.; Wavelength: 254 nm, R_{T}=7.170 min. ¹H NMR (400 MHz, DMSO-d₆) δ 7.83-7.74 (m, 4H), 7.62-7.50 (m, 2H), 7.44 (dd, J=8.4, 2.0 Hz, 1H), 7.35 (s, 2H), 5.89-5.23 (m, 2H), 4.63-4.43 (m, 2H), 4.22-4.06 (m, 1H), 3.84-3.69 (m, 1H), 3.14-3.05 (m, 1H), 2.95-2.89 (m, 1H), 2.66-2.58 (m, 1H), 1.63-1.51 (m, 3H), 1.26 (d, J=6.4 Hz, 3H), 1.21-1.06 (m, 3H).

### 16B:

LC-MS (ESI): R_{T}=3.189 min, mass calcd. for C₂₆H₂₇Cl₂N₅O₄S 575.1, m/z found 576.2 [M+H]⁺. Chiral analysis: Column: Chiralpak IE 5 μm, 4.6*250 mm; Mobile Phase: MeOH:EtOH=50:50 at 1 mL/min; Temp 30° C.; Wavelength: 254 nm, R_{T}=8.308 min. ¹H NMR (400 MHz, DMSO-d₆) δ 7.86-7.73 (m, 4H), 7.64-7.49 (m, 2H), 7.44 (dd, J=8.0, 1.2 Hz, 1H), 7.35 (s, 2H), 5.91-5.23 (m, 2H), 4.55-4.08 (m, 3H), 3.51-3.38 (m, 2H), 2.96-2.91 (m, 1H), 2.65-2.55 (m, 1H), 1.65-1.50 (m, 3H), 1.44 (d, J=6.4 Hz, 3H), 1.24-1.06 (m, 3H).

### Compounds 17A and 17B

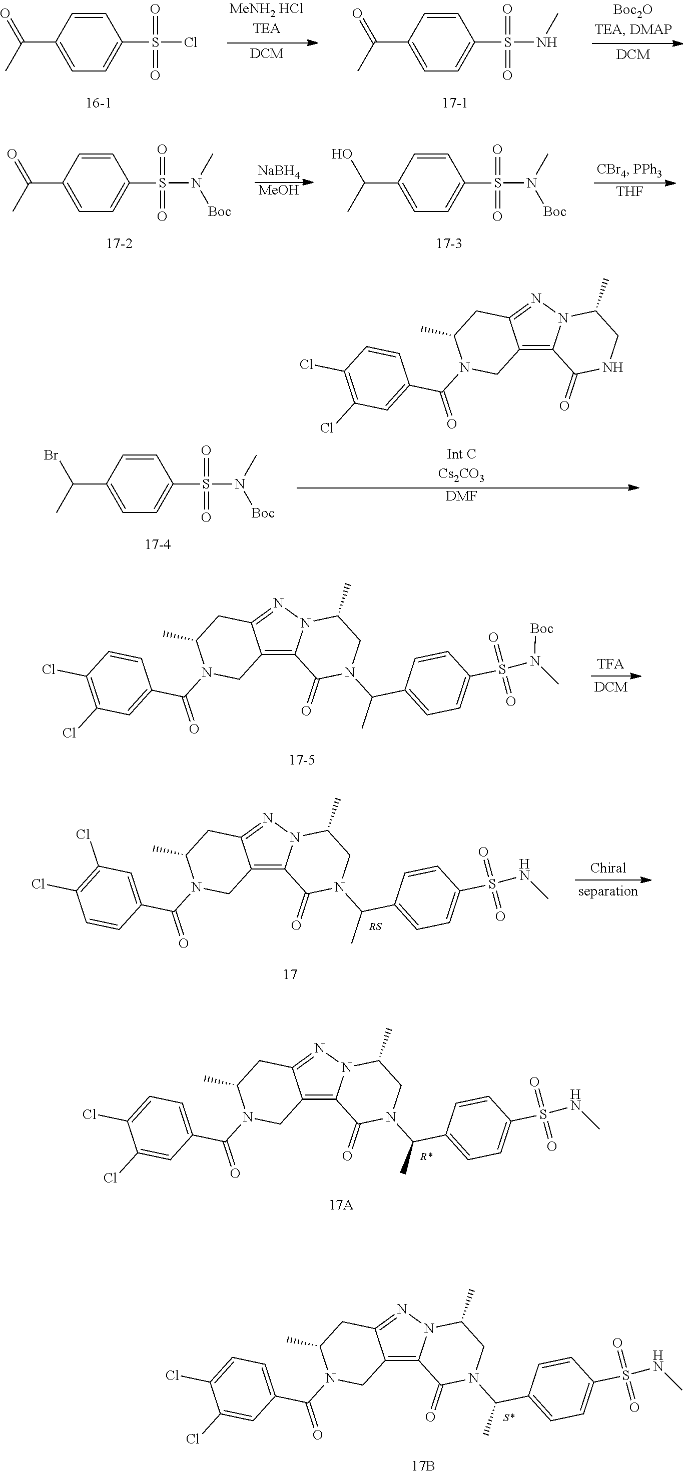

### Intermediate 17-1

### 4-Acetyl-N-methylbenzenesulfonamide

To a mixture of 4-acetylbenzenesulfonyl chloride 16-1 (3.0 g, 13.7 mmol) and methanamine hydrochloride (1.4 g, 20.7 mmol) in dichloromethane (30 mL) was added triethylamine (6 mL, 43.0 mmol) at 0° C. The mixture was stirred at room temperature for 5 hours. The mixture was poured into water (100 mL) and extracted with ethyl acetate (100 mL) for three times. The combined organic layers were washed with water (100 mL) twice, dried over Na₂SO₄₍ₛ₎, filtered, and concentrated. The residue was purified by silica gel column chromatography (ethyl acetate:petroleum ether=1:5) to give the title compound (1.1 g, 100% purity from LCMS, 37% yield) as yellow solids. LC-MS (ESI): R_{T}=1.29 min, mass calcd. for C₉H₁₁NO₃S 213.2 m/z found 212.0 [M−H]⁻.

### Intermediate 17-2

### tert-Butyl ((4-acetylphenyl)sulfonyl)(methyl)carbamate

To a solution of 4-acetyl-N-methylbenzenesulfonamide 17-1 (1.0 g, 4.69 mmol, 100% purity) and di-tert-butyl dicarbonate (1.3 g, 5.96 mmol) in dichloromethane (10 mL) were added triethylamine (1.3 mL, 9.33 mmol) and 4-dimethylaminopyridine (58 mg, 0.475 mmol). The mixture was stirred at room temperature for 3 hours. The reaction mixture was poured into water (100 mL) and extracted with dichloromethane (100 mL) for three times. The combined organic layers were washed with brine (100 mL), dried over Na₂SO₄₍ₛ₎, filtered, and concentrated in vacuum. The residue was purified by silica gel column chromatography (petroleum ether:ethyl acetate=8:1) to afford the title compound (1.5 g, 94% purity from LCMS, 96% yield) as white solids. LC-MS (ESI): R_{T}=1.61 min, mass calcd. for C₁₄H₁₅NO₅S 313.3 m/z found 258.0 [M-56+H]⁺.

### Intermediate 17-3

### tert-Butyl ((4-(1-hydroxyethyl)phenyl)sulfonyl)(methyl)carbamate

To a solution of tert-butyl (4-acetylphenyl)sulfonyl(methyl)carbamate 17-2 (1.5 g, 4.50 mmol, 94% purity) in methanol (15 mL) was added sodium borohydride (190 mg, 5.02 mmol). The mixture was stirred at room temperature for 1 hour. To the mixture was added water (100 mL) and extracted with ethyl acetate (100 mL) for three times. The combined organic layers were washed with brine (100 mL), dried over Na₂SO₄₍ₛ₎, filtered, and concentrated to get the desired compound (1.4 g, 93% yield, 95% purity from LCMS) as yellow solids. LC-MS (ESI): R_{T}=1.52 min, mass calcd. for C₁₄H₂₁NO₅S 315.4 m/z found 260.0 [M-56+H]⁺.

### Intermediate 17-4

### tert-Butyl ((4-(1-bromoethyl)phenyl)sulfonyl)(methyl)carbamate

To a solution of tert-butyl ((4-(1-hydroxyethyl)phenyl)sulfonyl)(methyl)carbamate 17-3 (1.3 g, 95% purity, 3.92 mmol) and carbon tetrabromide (1.9 g, 5.73 mmol) in tetrahydrofuran (13 mL) were added triphenylphosphine (1.5 g, 5.72 mmol) at 0° C. The mixture was stirred at room temperature for 5 hours. The mixture was purified by silica gel column chromatography (petroleum ether:ethyl acetate=10:1) to afford the title compound (1.3 g, 95% purity from ¹H NMR, 83% yield) as white solids. ¹H NMR (300 MHz, CDCl₃) δ 7.89-7.86 (m, 2H), 7.59-7.56 (m, 2H), 5.19 (q, J=6.6 Hz, 1H), 3.36 (s, 3H), 2.05 (d, J=6.9 Hz, 3H), 1.35 (s, 9H).

### Intermediate 17-5

### tert-Butyl ((4-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)sulfonyl)(methyl)carbamate

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (350 mg, 0.890 mmol, 100% purity) and tert-butyl (4-(1-bromoethyl)phenyl)sulfonyl(methyl)carbamate 17-4 (440 mg, 1.11 mmol, 95% purity) in N,N-dimethylformamide (6 mL) was added cesium carbonate (875 mg, 2.69 mmol). The mixture was stirred at 40° C. for 16 hours. The mixture was added water (100 mL) and extracted with ethyl acetate (100 mL) for three times. The combined organic layers were washed with brine (100 mL), dried over Na₂SO₄₍ₛ₎, filtered, and concentrated. The residue was purified by C18 column (acetonitrile:water=20% to 95%) to give the title compound (400 mg, 97% purity from LCMS, 63% yield) as yellow solids. LC-MS (ESI): R_{T}=1.82 min, mass calcd. for C₃₂H₃₇Cl₂N₅O₆S 689.2 m/z found 707.2 [M+18]⁺.

### Compound 17

### 4-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylbenzenesulfonamide

To a solution of tert-butyl ((4-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)sulfonyl)(methyl)carbamate 17-5 (400 mg, 97% purity, 0.562 mmol) in dichloromethane (5 mL) was added trifluoroacetic acid (1 mL). The mixture was stirred at room temperature for 2 hours. The mixture was basified to pH=8 with 2 M sodium bicarbonate aqueous solution and extracted with ethyl acetate (100 mL) for three times. The combined organic layers were washed with brine (100 mL), dried over Na₂SO₄₍ₛ₎, filtered, and concentrated to get desired product (360 mg, 99% yield, 92% purity from LCMS) as white solids. LC-MS (ESI): R_{T}=1.63 min, mass calcd. for C₂₇H₂₉Cl₂N₅O₄S 589.1 m/z found 590.1 [M+H]⁺.

### Compounds 17A and 17B

### 4-((R*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylbenzenesulfonamide (17A) and 4-((S*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylbenzenesulfonamide (17B)

A racemate of 4-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylbenzenesulfonamide 17 (400 mg, 92% purity, 0.610 mmol) was separated by chiral Prep. HPLC separation condition: (Column: Chiralpak IE 5 m 30*250 mm; Mobile Phase: ACN:IPA=90:10 at 25 mL/min; Temp: 30° C.; Wavelength: 254 nm) to afford the title compounds 17A (148 mg, 99.7% purity, 41% yield, 100% stereopure) and 17B (146 mg, 98% purity, 39.7% yield, 99.7% stereopure) as white solids.

### 17A:

LC-MS (ESI): R_{T}=3.890 min, mass calcd. for C₂₇H₂₉Cl₂N₅O₄S 589.1 m/z found 590.2 [M+H]⁺. Chiral analysis (Column: Chiralpak IE 5 m 4.6*250 mm; Mobile Phase: ACN:IPA=90:10 at 1 mL/min; Temp: 30° C.; Wavelength: 254 nm, R_{T}=8.706 min). ¹H NMR (400 MHz, CDCl₃) δ 7.87 (d, J=8.0 Hz, 2H), 7.54-7.50 (m, 4H), 7.28-7.25 (m, 1H), 6.22-6.04 (m, 1H), 5.79-5.30 (m, 1H), 4.91-4.28 (m, 4H), 3.69-3.56 (m, 1H), 3.15-2.96 (m, 2H), 2.68-2.67 (m, 4H), 1.63-1.60 (m, 3H), 1.27 (d, J=6.0 Hz, 6H).

### 17B:

LC-MS (ESI): R_{T}=3.546 min, mass calcd. for C₂₇H₂₉Cl₂N₅O₄S 589.1 m/z found 590.2 [M+H]⁺ Chiral analysis (Column: Chiralpak IE 5 m 4.6*250 mm; Mobile Phase: ACN:IPA=90:10 at 1 mL/min; Temp: 30° C.; Wavelength: 254 nm, R_{T}=11.05 min). ¹H NMR (400 MHz, CDCl₃) δ 7.85 (d, J=8.4 Hz, 2H), 7.54-7.45 (m, 4H), 7.31-7.26 (m, 1H), 6.24-5.99 (m, 1H), 5.84-5.32 (m, 1H), 4.97-4.21 (m, 4H), 3.34-2.91 (m, 3H), 2.69-2.68 (m, 4H), 1.64-1.63 (m, 3H), 1.55 (d, J=6.4 Hz, 3H), 1.32-1.20 (m, 3H).

### Compounds 18A, 18B, 18C and 18D

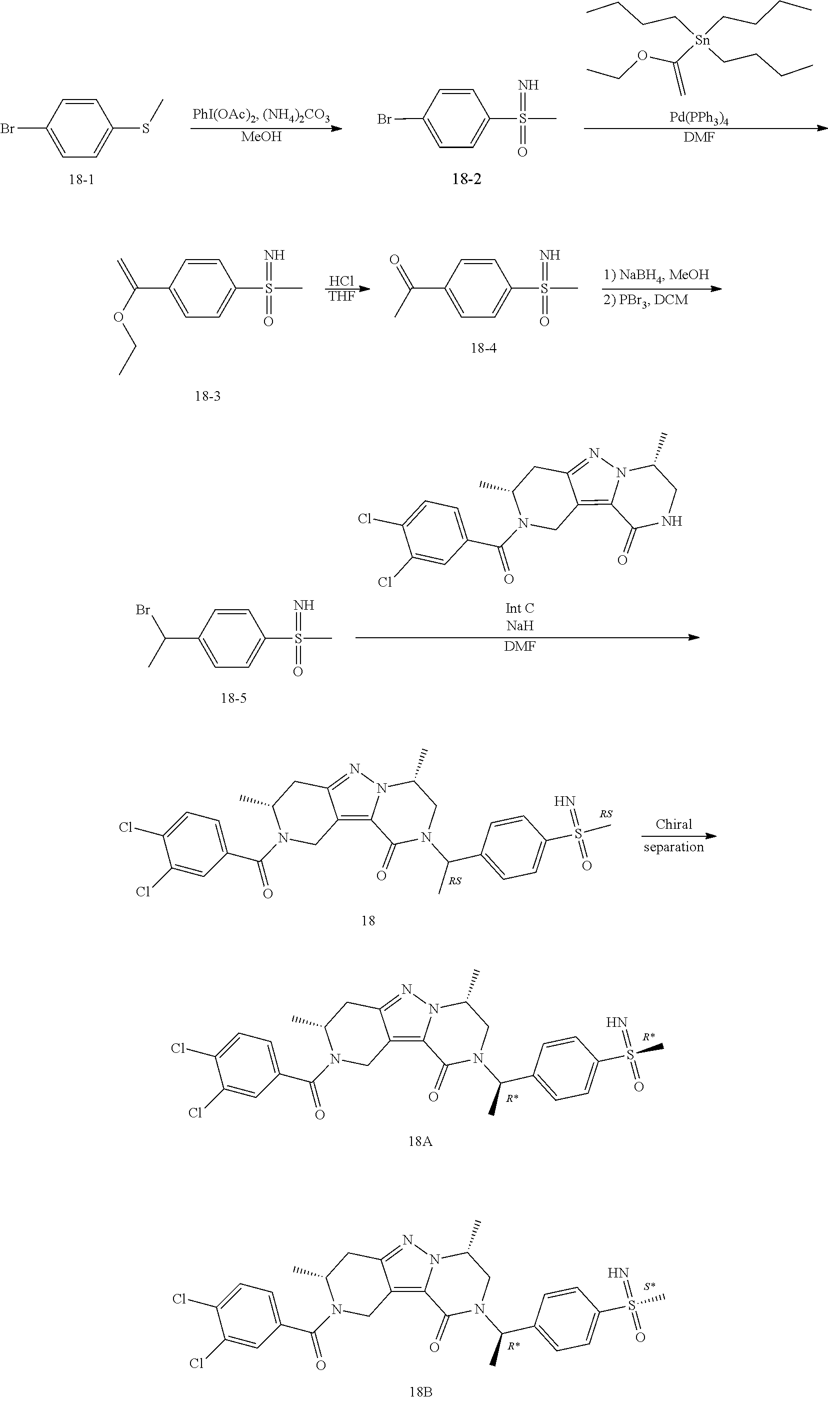

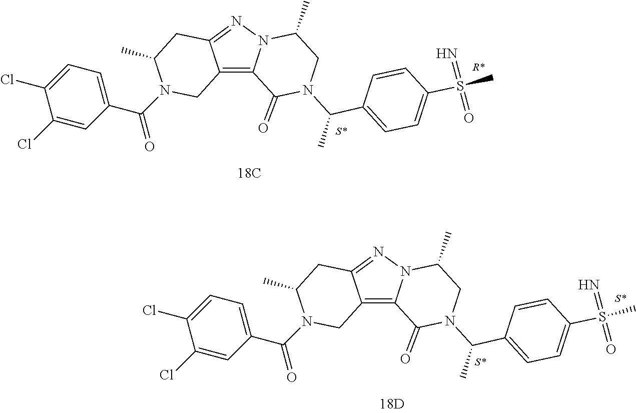 [SMILES: C[C@@H]1Cc2nn3c(c2CN1C(=O)c1ccc(Cl)c(Cl)c1)C(=O)N([C@@H](C)c1ccc([S@](C)(=N)=O)cc1)C[C@H]3C ; C[C@@H]1Cc2nn3c(c2CN1C(=O)c1ccc(Cl)c(Cl)c1)C(=O)N([C@@H](C)c1ccc([S@@](C)(=N)=O)cc1)C[C@H]3C]

### Intermediate 18-2

### (4-Bromophenyl)(imino)(methyl)-sulfanone

A solution of (4-bromophenyl)(methyl)sulfane 18-1 (1 g, 4.92 mmol), (Diacetoxyiodo)benzene (3.7 g, 11.5 mmol) and ammonium carbonate (710 mg, 7.39 mmol) in methanol (40 mL) was stirred at room temperature for 30 minutes under nitrogen atmosphere. The mixture was concentrated in vacuum to give a residue, which was purified by silica gel column chromatography (petroleum ether:ethyl acetate=10:1 to 4:1) to give the title compound (1.1 g, 100% purity from LCMS, 95% yield) as white solids. LC-MS (ESI): R_{T}=1.23 min, mass calcd. for C₇H₈BrNOS 232.9 m/z found 233.9 [M+H]⁺.

### Intermediate 18-3

### (4-(1-Ethoxyvinyl)phenyl)(imino)(methyl)-sulfanone

To a solution of (4-bromophenyl)(imino)(methyl)-sulfanone 18-2 (1.1 g, 100% purity, 4.70 mmol) and tributyl(1-ethoxyvinyl)stannane (2.5 mL, 7.4 mmol) in N,N-dimethylformamide (11 mL) was added tetrakis(triphenylphosphine)palladium (300 mg, 0.26 mmol) under nitrogen atmosphere. After stirred at 80° C. for 3 hours, the reaction mixture was quenched with water (50 mL) and extracted with ethyl acetate (30 mL) for three times. The combined organic layers were washed with brine (200 mL), dried over Na₂SO₄₍ₛ₎ and filtered. The filtrate was concentrated under reduced pressure to give a residue, which was purified by silica gel column chromatography (dichloromethane: ethyl acetate=20:1 to 10:1) to give the title compound (1 g, 94% purity from LCMS, 89% yield) as yellow solids. LC-MS (ESI): R_{T}=1.38 min, mass calcd. for C₁₁H₁₅NO₂S 225.1 m/z found 226.1 [M+H]⁺.

### Intermediate 18-4

### (4-Acetylphenyl)(imino)(methyl)-sulfanone

To a solution of (4-(1-ethoxyvinyl)phenyl)(imino)(methyl)-sulfanone 18-3 (1 g, 94% purity, 4.17 mmol) in tetrahydrofuran (10 mL) was added 3 M Hydrochloric acid in water (5 mL, 15 mmol) at 0° C. After stirred at room temperature for 3 hours, the reaction mixture was quenched with saturated sodium carbonate to pH=9˜10 at 0° C. The aqueous layer was extracted with ethyl acetate (30 mL) twice. The combined organic layers were washed with brine (20 mL) twice, dried over Na₂SO₄₍ₛ₎ and filtered. The filtrate was concentrated to give a residue, which was purified by silica gel column chromatography (dichloromethane: ethyl acetate=20:1 to 10:1) to give the title compound (550 mg, 90% purity from LCMS, 60% yield) as yellow solids. LC-MS (ESI): R_{T}=0.32 min, mass calcd. for C₉H₁₁NO₂S 197.1 m/z found 198.1 [M+H]⁺.

### Intermediate 18-5

### (4-(1-Bromoethyl)phenyl)(imino)(methyl)-sulfanone

To a suspension of (4-acetylphenyl)(imino)(methyl)-sulfanone 18-4 (550 mg, 90% purity, 2.51 mmol) in methanol (5 mL) was added sodium borohydride (63 mg, 1.67 mmol) at 0° C. under nitrogen atmosphere. After stirred for 30 minutes at 0° C., the reaction mixture was concentrated under reduced pressure to give a residue, the residue was diluted with ethyl acetate (20 mL) then washed with water (50 mL) twice and dried over Na₂SO₄₍ₛ₎. The filtrate was concentrated under reduced pressure to give a residue, which was diluted in dichloromethane (5 mL). Phosphorus(III) Bromide (900 mg, 3.33 mmol) was added at 0° C. under nitrogen atmosphere. After stirred for 1 hour at room temperature, the reaction mixture was concentrated under reduced pressure to give a residue. The residue was diluted with ethyl acetate (50 mL) then washed with saturated sodium bicarbonate solution (50 mL) twice and dried over Na₂SO₄₍ₛ₎. The filtrate was concentrated under reduced pressure to give title compound (420 mg, 95% purity from LCMS, 55% yield) as yellow oil. LC-MS (ESI): R_{T}=1.30 min, mass calcd. for C₉H₁₂BrNOS 260.9 m/z found 262.0 [M+H]⁺.

### Compound 18

### (3R,7R)-2-(3,4-dichlorobenzoyl)-3,7-dimethyl-9-(1-(4-(S-methylsulfonimidoyl)phenyl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (320 mg, 100% purity, 0.814 mmol) in N,N-dimethylformamide (5 mL) was added 60% wt. sodium hydride in mineral oil (75 mg, 1.72 mmol). After stirred at 0° C. for 30 minutes, (4-(1-bromoethyl)phenyl)(imino)(methyl)-sulfanone 18-5 (320 mg, 95% purity, 1.16 mmol) was added to the mixture. After stirred at 0° C. for 1 hour, the reaction mixture was quenched by brine (50 mL) and extracted with ethyl acetate (20 mL) for three times. The combined organic layers were washed with water (100 mL) and brine (100 mL), dried over Na₂SO₄₍ₛ₎, filtered and concentrated under reduced pressure to give a residue, which was purified by silica gel column chromatography (dichloromethane:acetone=10:1 to 4:1) to give the title compound (370 mg, 96% purity from LCMS, 76% yield) as white solids. LC-MS (ESI): R_{T}=1.49 min, mass calcd. for C₂₇H₂₉Cl₂N₅O₃S 573.1 m/z found 574.1 [M+H]⁺.

### Compounds 18A, 18B, 18C and 18D

### (3R,7R)-2-(3,4-dichlorobenzoyl)-3,7-dimethyl-9-((R*)-1-(4-((R*)—S-methylsulfonimidoyl)phenyl)ethyl)-1,2,3,4,8,9-hexahydropyrido[4′,3′:3,4]pyrazolo[1,5-a]pyrazin-10(7H)-one (18A), (3R,7R)-2-(3,4-dichlorobenzoyl)-3,7-dimethyl-9-((R*)-1-(4-((S*)—S-methylsulfonimidoyl)phenyl)ethyl)-1,2,3,4,8,9-hexahydropyrido[4′,3′:3,4]pyrazolo[1,5-a]pyrazin-10(7H)-one (18B), (3R,7R)-2-(3,4-dichlorobenzoyl)-3,7-dimethyl-9-((S*)-1-(4-((R*)—S-methylsulfonimidoyl)phenyl)ethyl)-1,2,3,4,8,9-hexahydropyrido[4′,3′:3,4]pyrazolo[1,5-a]pyrazin-10(7H)-one (18C) and (3R,7R)-2-(3,4-dichlorobenzoyl)-3,7-dimethyl-9-((S*)-1-(4-((S*)—S-methylsulfonimidoyl)phenyl)ethyl)-1,2,3,4,8,9-hexahydropyrido[4′,3′:3,4]pyrazolo[1,5-a]pyrazin-10(7H)-one (18D)

A racemic mixture of (3R,7R)-2-(3,4-dichlorobenzoyl)-3,7-dimethyl-9-(1-(4-(S-methylsulfonimidoyl)phenyl)ethyl)-1,2,3,4,8,9-hexahydropyrido[4′,3′:3,4]pyrazolo[1,5-a]pyrazin-10(7H)-one 18 (370 mg, 96% stereopure, 0.618 mmol) was separated by chiral prep.HPLC (separation condition: Column: Chiralpak IE 5 μm 20*250 mm; Mobile Phase: MeOH:DCM=70:30 at 15 mL/min; Temp: 30° C.; Wavelength: 254 nm) to give the mixture compounds Fraction I (140 mg, 100% purity, 99.9% stereopure, 39% yield) as white solids and Fraction 11 (140 mg, 100% purity, 100% stereopure, 39% yield) as white solids. Fraction I (140 mg, 100% purity, 99.9% stereopure, 39% yield) was separated by chiral prep.HPLC (separation condition: Column: Chiral Pak IH 5 μm 20*250 mm; Mobile Phase: ACN:IPA=70:30; at 15 mL/min; Temp: 30° C.; Wavelength: 254 nm) to give the title compounds 18A (45.2 mg, 99.2% purity from LCMS, 99.8% stereopure, 32% yield) as white solids and 18B (39.7 mg, 99.2% purity from LCMS, 28% yield, 100% stereopure) as white solids. Fraction II (140 mg, 100% purity, 0.244 mmol) was separated by chiral prep.HPLC (separation condition: Column: Chiralpak IC 5 μm 20*250 mm; Mobile Phase: DCM:MeOH:DEA=95:5:0.1 at 60 mL/min; Temp: 38° C.; Wavelength: 254 nm) to give the title compounds 18C (34.3 mg, 99.0% purity from LCMS, 25% yield, 100% stereopure) as white solids and 18D (36.2 mg, 98.3% purity from LCMS, 26% yield, 99.7% stereopure) as white solids.

### 18A:

LC-MS (ESI): R_{T}=3.423 min, mass calcd. for C₂₇H₂₉Cl₂N₅O₃S 573.1 m/z found 574.2 [M+H]⁺. Chiral analysis (Chiralpak IH 5 m 4.6*250 mm; Mobile Phase: ACN:IPA=70:30 at 1 mL/min; Temp: 30° C.; Wavelength: 254 nm, R_{T}=5.273 min). ¹H NMR (400 MHz, CDCl₃) δ 8.02 (d, J=8.4 Hz, 2H), 7.57-7.50 (m, 4H), 7.28-7.25 (m, 1H), 6.13 (br s, 1H), 5.75-5.43 (m, 1H), 4.90-4.40 (m, 3H), 3.66-3.62 (m, 1H), 3.11-2.97 (m, 5H), 2.69-2.65 (m, 2H), 1.64 (s, 3H), 1.31-1.27 (m, 6H).

### 18B:

LC-MS (ESI): R_{T}=3.417 min, mass calcd. for C₂₇H₂₉Cl₂N₅O₃S 573.1 m/z found 574.2 [M+H]⁺. Chiral analysis (Chiralpak IH 5 m 4.6*250 mm; Mobile Phase: ACN:IPA=70:30 at 1 mL/min; Temp: 30° C.; Wavelength: 254 nm, R_{T}=7.818 min). ¹H NMR (400 MHz, CDCl₃) δ 8.02 (d, J=8.0 Hz, 2H), 7.57-7.50 (m, 4H), 7.28-7.25 (m, 1H), 6.13 (br s, 1H), 5.77-5.40 (m, 1H), 4.86-4.26 (m, 3H), 3.66-3.61 (m, 1H), 3.11 (s, 3H), 3.02-2.97 (m, 2H), 2.71-2.64 (m, 2H), 1.67-1.61 (m, 3H), 1.30-1.26 (m, 6H).

### 18C:

LC-MS (ESI): R_{T}=2.968 min, mass calcd. for C₂₇H₂₉Cl₂N₅O₃S 573.1 m/z found 574.1 [M+H]⁺. Chiral analysis (Chiralpak IC 5 m 4.6*250 mm; Mobile Phase: DCM:MeOH:DEA=95:5:0.1 at 1 mL/min; Temp: 30° C.; Wavelength: 254 nm, R_{T}=3.182 min). ¹H NMR (400 MHz, CDCl₃) δ 8.01 (d, J=8.4 Hz, 2H), 7.54-7.50 (m, 4H), 7.28-7.27 (m, 1H), 6.19-6.04 (m, 1H), 5.77-5.42 (m, 1H), 4.90-4.30 (m, 3H), 3.36-3.24 (m, 2H), 3.12-2.97 (m, 4H), 2.70-2.64 (m, 2H), 1.64 (d J=6.8 Hz, 3H), 1.55 (d, J=6.8 Hz, 3H), 1.28-1.24 (m, 3H).

### 18D:

LC-MS (ESI): R_{T}=2.970 min, mass calcd. for C₂₇H₂₉Cl₂N₅O₃S 573.1 m/z found 574.1 [M+H]⁺. Chiral analysis (Chiralpak IC 5 m 4.6*250 mm; Mobile Phase: DCM:MeOH:DEA=95:5:0.1 at 1 mL/min; Temp: 30° C.; Wavelength: 254 nm, R_{T}=3.526 min). ¹H NMR (400 MHz, CDCl₃) δ 8.01 (d, J=8.0 Hz, 2H), 7.54-7.50 (m, 4H), 7.3-7.27 (m, 1H), 6.18-6.03 (m, 1H), 5.75-5.41 (m, 1H), 4.86-4.31 (m, 3H), 3.35-3.26 (m, 2H), 3.12 (s, 3H), 3.08-2.94 (m, 1H), 2.70-2.64 (m, 2H), 1.64 (d, J=7.2 Hz, 3H), 1.55 (d, J=6.0 Hz, 3H), 1.26 (br s, 3H).

### Compounds 19A, 19B, 19C and 19D

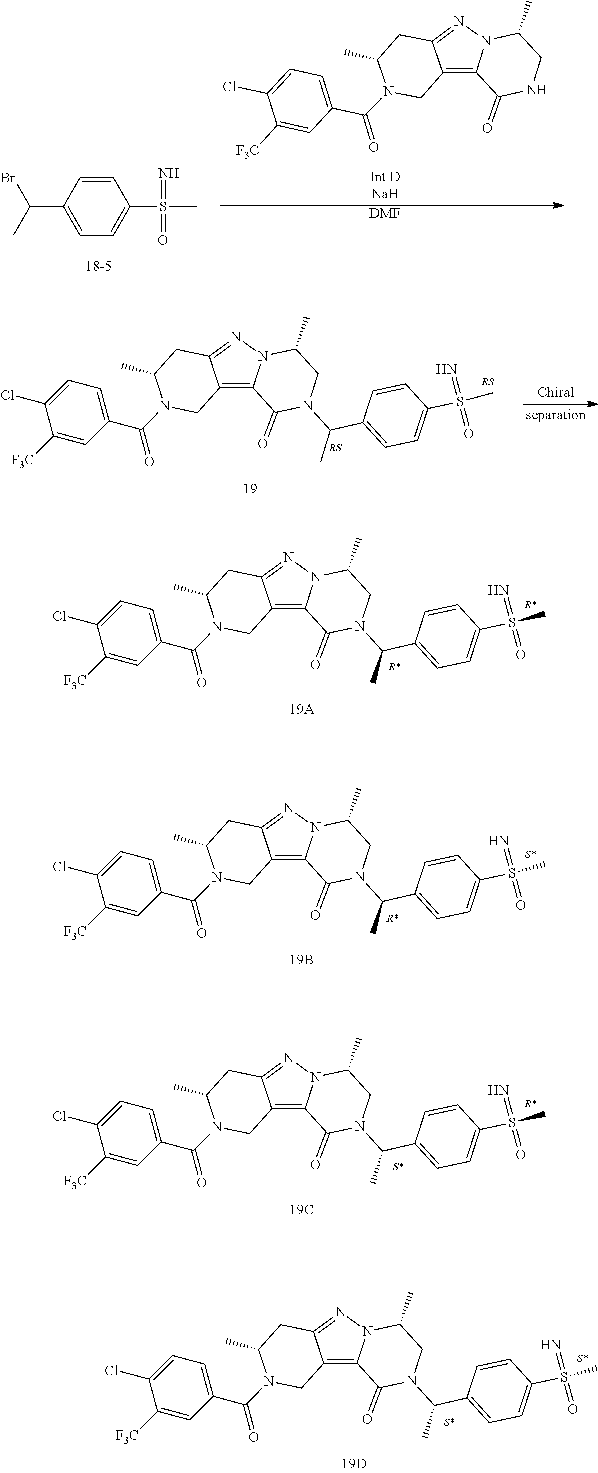

### Compound 19

### (3R,7R)-2-(4-Chloro-3-(trifluoromethyl)benzoyl)-3,7-dimethyl-9-(1-(4-(S-methylsulfonimidoyl)phenyl)ethyl)-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one Int D (350 mg, 90% purity, 0.738 mmol) in N,N-dimethylformamide (10 mL) was added 60% sodium hydride in mineral oil (100 mg, 2.29 mmol). After stirred at 0° C. for 30 minutes, 1-(1-bromoethyl)-4-(S-methylsulfonimidoyl)benzene 18-5 (270 mg, 80% purity, 0.824 mmol) was added to the mixture. After stirred at 0° C. for 1 hour, the reaction mixture was quenched by brine (50 mL) and extracted with ethyl acetate (20 mL) for three times. The combined organic layers were washed by water (50 mL) and brine (50 mL), dried over Na₂SO₄₍ₛ₎, filtered and concentrated in vacuum to give a residue, which was purified by silica gel column chromatography (dichloromethane:acetone=10:1 to 4:1) to give the title compound (350 mg, 94% purity from LCMS, 73% yield) as white solids. LC-MS (ESI): R_{T}=1.53 min, mass calcd. for C₂₈H₂₉ClF₃N₅O₃S 607.1 m/z found 608.1 [M+H]⁺.

### Compounds 19A, 19B, 19C and 19D

### (3R,7R)-2-(4-chloro-3-(trifluoromethyl)benzoyl)-3,7-dimethyl-9-((R*)-1-(4-((R*)—S-methylsulfonimidoyl)phenyl)ethyl)-1,2,3,4,8,9-hexahydropyrido[4′,3′:3,4]pyrazolo[1,5-a]pyrazin-10(7H)-one (19A), (3R,7R)-2-(4-chloro-3-(trifluoromethyl)benzoyl)-3,7-dimethyl-9-((R*)-1-(4-((S*)—S-methylsulfonimidoyl)phenyl)ethyl)-1,2,3,4,8,9-hexahydropyrido[4′,3′:3,4]pyrazolo[1,5-a]pyrazin-10(7H)-one (19B), (3R,7R)-2-(4-chloro-3-(trifluoromethyl)benzoyl)-3,7-dimethyl-9-((S*)-1-(4-((R*)—S-methylsulfonimidoyl)phenyl)ethyl)-1,2,3,4,8,9-hexahydropyrido[4′,3′:3,4]pyrazolo[1,5-a]pyrazin-10(7H)-one (19C) and (3R,7R)-2-(4-chloro-3-(trifluoromethyl)benzoyl)-3,7-dimethyl-9-((S*)-1-(4-((S*)—S-methylsulfonimidoyl)phenyl)ethyl)-1,2,3,4,8,9-hexahydropyrido[4′,3′:3,4]pyrazolo[1,5-a]pyrazin-10(7H)-one (19D)

A racemic mixture of (3R,7R)-2-(4-chloro-3-(trifluoromethyl)benzoyl)-3,7-dimethyl-9-(1-(4-(S-methylsulfonimidoyl)phenyl)ethyl)-1,2,3,4,8,9-hexahydropyrido[4′,3′:3,4]pyrazolo[1,5-a]pyrazin-10(7H)-one 19 (350 mg, 94% purity, 0.541 mmol) was separated by chiral Prep. HPLC separation condition: (Column: Chiralpak IA 5 μm 20*250 mm; Mobile Phase: CO₂:EtOH=60:40; 8 g/min; Col. Temp: 40° C.; Wavelength: 230 nm, Back pressure: 100 bar) to give Fraction I (180 mg, 98% purity, 100% stereopure, 51% yield) as white solids and the title compounds 19C (48.1 mg, 99.1% purity, 13% yield, 100% stereopure) as white solids and 19D (37.4 mg, 99.2% purity from LCMS, 10% yield, 99.5% stereopure) as white solids. Fraction I was separated by chiral prep. HPLC (separation condition: Column: Chiralpak IH 5 μm 20*250 mm; Mobile Phase: ACN:IPA:=90:10; at 15 mL/min; Temp: 30° C.; Wavelength: 254 nm) to give the title compounds 19A (50.2 mg, 99.2% purity, 27% yield, 100% stereopure) as white solids and 19B (46 mg, 99.1% purity, 25% yield, 99.9% stereopure) as white solids.

### 19A:

LC-MS (ESI): R_{T}=3.288 min, mass calcd. for C₂₈H₂₉ClF₃N₅O₃S 607.1 m/z found 608.2 [M+H]⁺. Chiral analysis (Chiralpak IH 5 m 4.6*250 mm; Mobile Phase: ACN:IPA=90:10 at 1 mL/min; Temp: 30° C.; Wavelength: 254 nm, R_{T}=5.030 min). ¹H NMR (400 MHz, CDCl₃) δ 8.03 (d, J=8.4 Hz, 2H), 7.78 (d, J=0.8 Hz, 1H), 7.59-7.53 (m, 4H), 6.18-6.06 (m, 1H), 5.72-5.43 (m, 1H), 4.88-4.64 (m, 1H), 4.49-4.28 (m, 2H), 3.69-3.55 (m, 1H), 3.12 (s, 3H), 3.07-2.97 (m, 2H), 2.70-2.61 (m, 2H), 1.63 (d J=6.4 Hz, 3H), 1.32-1.26 (m, 6H). ¹⁹F NMR (376 MHz, CDCl₃) δ −62.82.

### 19B:

LC-MS (ESI): R_{T}=3.288 min, mass calcd. for C₂₈H₂₉ClF₃N₅O₃S 607.1 m/z found 608.2 [M+H]⁺. Chiral analysis (Chiralpak IH 5 m 4.6*250 mm; Mobile Phase: ACN:IPA=90:10 at 1 mL/min; Temp: 30° C.; Wavelength: 254 nm, R_{T}=6.670 min). ¹H NMR (400 MHz, CDCl₃) δ 8.03 (d J=8.4 Hz, 2H), 7.78 (d J=1.2 Hz, 1H), 7.59-7.53 (m, 4H), 6.21-6.04 (m, 1H), 5.74-5.26 (m, 1H), 5.01-4.61 (m, 1H), 4.51-4.28 (m, 2H), 3.71-3.54 (m, 1H), 3.12 (s, 3H), 3.06-2.97 (m, 2H), 2.73-2.66 (m, 2H), 1.63 (d J=6.4 Hz, 3H), 1.31-1.26 (m, 6H). ¹⁹F NMR (376 MHz, CDCl₃) δ −62.82.

### 19C:

LC-MS (ESI): R_{T}=3.270 min, mass calcd. for C₂₈H₂₉ClF₃N₅O₃S 607.1 m/z found 608.1 [M+H]⁺. Chiral analysis (Chiralpak IA 5 m 4.6*250 mm; Mobile Phase: CO₂:EtOH=60:40 at 4 g/min; Col. Temp: 40° C.; Wavelength: 214 nm, Back pressure: 100 bar, R_{T}=6.3 min). ¹H NMR (400 MHz, CDCl₃) δ 8.01 (d, J=8.4 Hz, 2H), 7.79 (s, 1H), 7.60-7.51 (m, 4H), 6.21-6.00 (m, 1H), 5.75-5.42 (m, 1H), 4.83-4.30 (m, 3H), 3.36-3.24 (m, 2H), 3.11 (s, 3H), 3.06-2.92 (m, 1H), 2.76-2.65 (m, 2H), 1.64 (d, J=6.8 Hz, 3H), 1.55 (d, J=6.4 Hz, 3H), 1.29-1.25 (m, 3H). ¹⁹F NMR (376 MHz, CDCl₃) δ −62.81.

### 19D:

LC-MS (ESI): R_{T}=3.257 min, mass calcd. for C₂₈H₂₉ClF₃N₅O₃S 607.1 m/z found 608.1 [M+H]⁺. Chiral analysis (Chiralpak IA 5 m 4.6*250 mm; Mobile Phase: CO₂:EtOH=60:40 at 4 g/min; Col. Temp: 40° C.; Wavelength: 214 nm, Back pressure: 99 bar, R_{T}=8.08 min). ¹H NMR (400 MHz, CDCl₃) δ 8.01 (d, J=8.0 Hz, 2H), 7.79 (s, 1H), 7.60-7.52 (m, 4H), 6.19-6.02 (m, 1H), 5.79-5.44 (m, 1H), 4.82-4.30 (m, 3H), 3.36-3.27 (m, 2H), 3.12 (s, 3H), 3.06-2.96 (m, 1H), 2.75-2.67 (m, 2H), 1.64 (d, J=6.8 Hz, 3H), 1.55 (d, J=6.4 Hz, 3H), 1.29-1.25 (m, 3H). ¹⁹F NMR (376 MHz, CDCl₃) δ −62.81.

### Compounds 20A, 20B, 20C and 20D

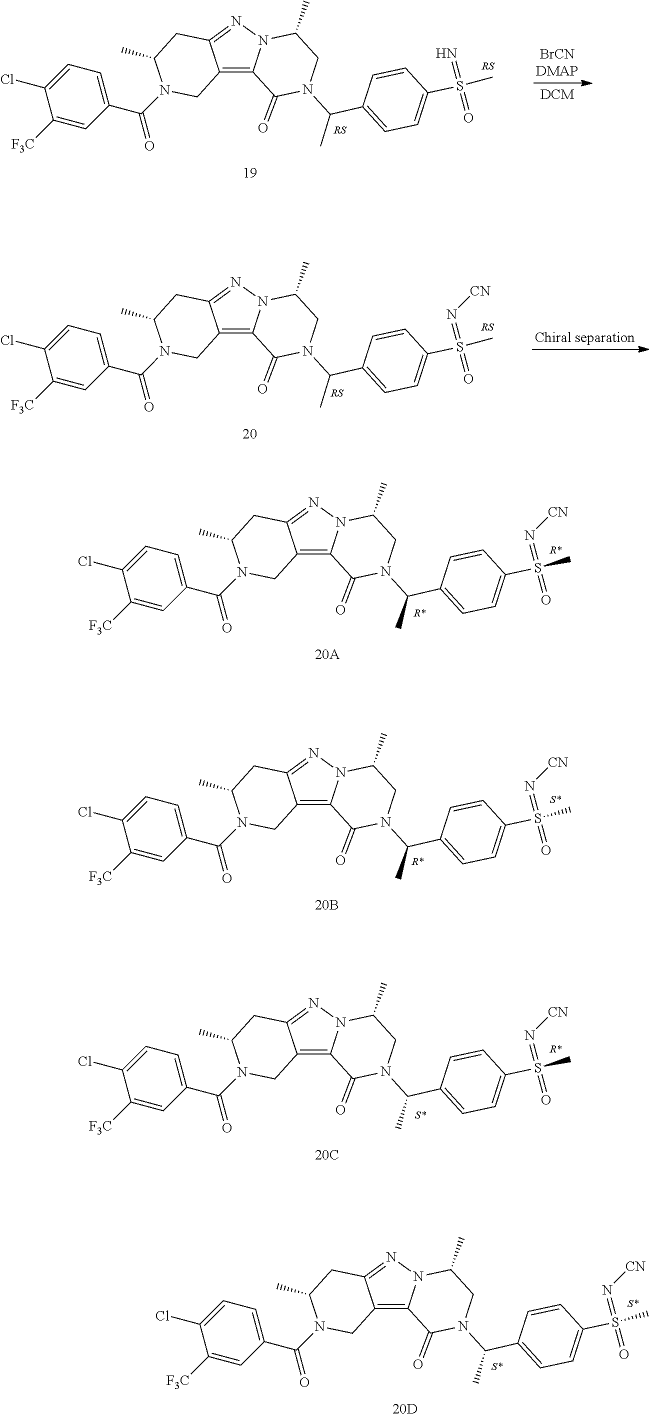

### Compound 20

### N-((4-(1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide

To a solution of (3R,7R)-2-(4-chloro-3-(trifluoroamethyl)benzoyl)-3,7-dimethyl-9-(1-(4-(S-methylsulfonimidoyl)phenyl)ethyl)-1,2,3,4,8,9-hexahydropyrido[4′,3′:3,4]pyrazolo[1,5-a]pyrazin-10(7H)-one 19 (300 mg, 100% purity, 0.493 mmol), 4-dimethylaminopyridine (300 mg, 2.46 mmol) in dichloromethane (20 mL) was added carbononitridic bromide (240 mg, 2.27 mmol) at room temperature. After stirred at room temperature for 6 hours, the reaction mixture was concentrated, quenched by brine (50 mL) and extracted with ethyl acetate (50 mL) for three times. The combined organic layers were washed by water (100 mL) and brine (100 mL), dried over Na₂SO₄, filtered and concentrated in vacuum to give a residue, which was purified by silica gel column chromatography (dichloromethane: ethyl acetate=10:1 to 4:1) to give the title compound (180 mg, 92% purity from LCMS, 53% yield) as white solids. LC-MS (ESI): R_{T}=1.58 min, mass calcd. for C₂₉H₂₈ClF₃N₆O₃S 632.1 m/z found 633.0 [M+H]⁺.

### Compounds 20A, 20B, 20C and 20D

### N—((R*)-(4-((R*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide (20A), N—((S*)-(4-((R*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide (20B), N—((R*)-(4-((S*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide (20C) and N—((S*)-(4-((S*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide (20D)

A racemic mixture of N-((4-(1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)phenyl)(methyl)(oxo)-16-sulfaneylidene)cyanamide 20 (180 mg, 92% purity, 0.262 mmol) was separated by chiral prep. HPLC (separation condition: Column: Chiralpak IF 5 m 20*250 mm; Mobile Phase: ACN:IPA:=90:10 at 15 mL/min; Temp: 30° C.; Wavelength: 254 nm) to give Fraction I (100 mg, 96% purity from LCMS, 55% yield, 99.9% stereopure) as white solids and 20D (29.4 mg, 99.6% purity, 16% yield, 100% stereopure) as white solids. Fraction I was separated by chiral prep. HPLC (separation condition: Column: Chiralpak IC 5 m 20*250 mm; Mobile Phase: ACN=100 at 15 mL/min; Temp: 30° C.; Wavelength: 254 nm) to give 20B (13.5 mg, 99.7% purity, 14% yield, 100% stereopure) as white solids and Fraction II (55 mg, 95% purity from LCMS, 55% yield, 100% stereopure) as white solids. Fraction II was separated by chiral prep.HPLC (separation condition: Column: Chiralpak IB N-5 5 m 20*250 mm; Mobile Phase: ACN=100 at 15 mL/min; Temp: 30° C.; Wavelength: 254 nm) to give the title compounds 20A (17 mg, 99.2% purity, 31% yield, 99.9% stereopure) as white solids and 20C (23 mg, 99.5% purity, 41% yield, 99.9% stereopure) as white solids.

### 20A:

LC-MS (ESI): R_{T}=3.559 min, mass calcd. for C₂₉H₂₈ClF₃N₆O₃S 632.1 m/z found 633.3 [M+H]⁺. Chiral analysis (Chiralpak IF 250 mm*4.6 mm 5 m: ACN:IPA=90:10 at 1 mL/min; Temp: 30° C.; Wavelength: 254 nm, R_{T}=4.793 min). ¹H NMR (400 MHz, CDCl₃) δ 8.00 (d, J=8.4 Hz, 2H), 7.79 (s, 1H), 7.73-7.69 (m, 2H), 7.59-7.53 (m, 2H), 6.20-6.08 (m, 1H), 5.72-5.19 (m, 1H), 4.88-4.66 (m, 1H), 4.44-4.17 (m, 2H), 3.68-3.58 (m, 1H), 3.34 (s, 3H), 3.15-2.92 (m, 2H), 2.71-2.64 (m, 1H), 1.66 (d, J=5.2 Hz, 3H), 1.38 (d J=6.4 Hz, 3H), 1.30-1.25 (m, 3H). ¹⁹F NMR (376 MHz, CDCl₃) δ −62.79.

### 20B:

LC-MS (ESI): R_{T}=3.560 min, mass calcd. for C₂₉H₂₈ClF₃N₆O₃S 632.1 m/z found 633.3 [M+H]⁺. Chiral analysis (Chiralpak IF 250 mm*4.6 mm 5 m: ACN:IPA=90:10 at 1 mL/min; Temp: 30° C.; Wavelength: 254 nm, R_{T}=5.097 min). ¹H NMR (400 MHz, CDCl₃) δ 8.00 (d, J=8.4 Hz, 2H), 7.78 (s, 1H), 7.72-7.68 (m, 2H), 7.59-7.52 (m, 2H), 6.18-6.05 (m, 1H), 5.74-5.26 (m, 1H), 4.96-4.65 (m, 1H), 4.44-4.17 (m, 2H), 3.73-3.55 (m, 1H), 3.34 (s, 3H), 3.15-2.94 (m, 2H), 2.71-2.64 (m, 1H), 1.66 (d, J=6.8 Hz, 3H), 1.37 (d J=6.4 Hz, 3H), 1.28-1.25 (m, 3H). ¹⁹F NMR (376 MHz, CDCl₃) δ −62.79.

### 20C:

LC-MS (ESI): R_{T}=3.594 min, mass calcd. for C₂₉H₂₈ClF₃N₆O₃S 632.1 m/z found 633.3 [M+H]⁺. Chiral analysis (Chiralpak IF 250 mm*4.6 mm 5 m: ACN:IPA=90:10 at 1 mL/min; Temp: 30° C.; Wavelength: 254 nm, R_{T}=5.697 min). ¹H NMR (400 MHz, CDCl₃) δ7.99 (d, J=8.4 Hz, 2H), 7.78 (s, 1H), 7.68-7.65 (m, 2H), 7.60-7.54 (m, 2H), 6.21-6.04 (m, 1H), 5.77-5.33 (m, 1H), 4.87-4.52 (m, 1H), 4.41-4.26 (m, 2H), 3.40-3.35 (m, 1H), 3.33 (s, 3H), 3.28-3.24 (m, 1H), 3.12-2.99 (m, 1H), 2.75-2.68 (m, 1H), 1.67 (d, J=6.8 Hz, 3H), 1.59 (d J=6.4 Hz, 3H), 1.29-1.25 (m, 3H). ¹⁹F NMR (376 MHz, CDCl₃) δ −62.78.

### 20D:

LC-MS (ESI): R_{T}=3.592 min, mass calcd. for C₂₉H₂₈ClF₃N₆O₃S 632.1 m/z found 633.0 [M+H]⁺. Chiral analysis (Chiralpak IF 250 mm*4.6 mm 5 m: ACN:IPA=90:10 at 1 mL/min; Temp: 30° C.; Wavelength: 254 nm, R_{T}=6.771 min). ¹H NMR (400 MHz, CDCl₃) δ 7.99 (d, J=8.4 Hz, 2H), 7.79 (s, 1H), 7.68-7.64 (m, 2H), 7.60-7.54 (m, 2H), 6.20-6.04 (m, 1H), 5.79-5.35 (m, 1H), 4.99-4.59 (m, 1H), 4.46-4.28 (m, 2H), 3.40-3.35 (m, 1H), 3.30 (s, 3H), 3.31-3.28 (m, 1H), 3.16-2.97 (m, 1H), 2.75-2.68 (m, 1H), 1.67 (d, J=6.8 Hz, 3H), 1.59 (br s, 3H), 1.29-1.25 (m, 3H). ¹⁹F NMR (376 MHz, CDCl₃) δ −62.77.

### Compounds 21A and 21B

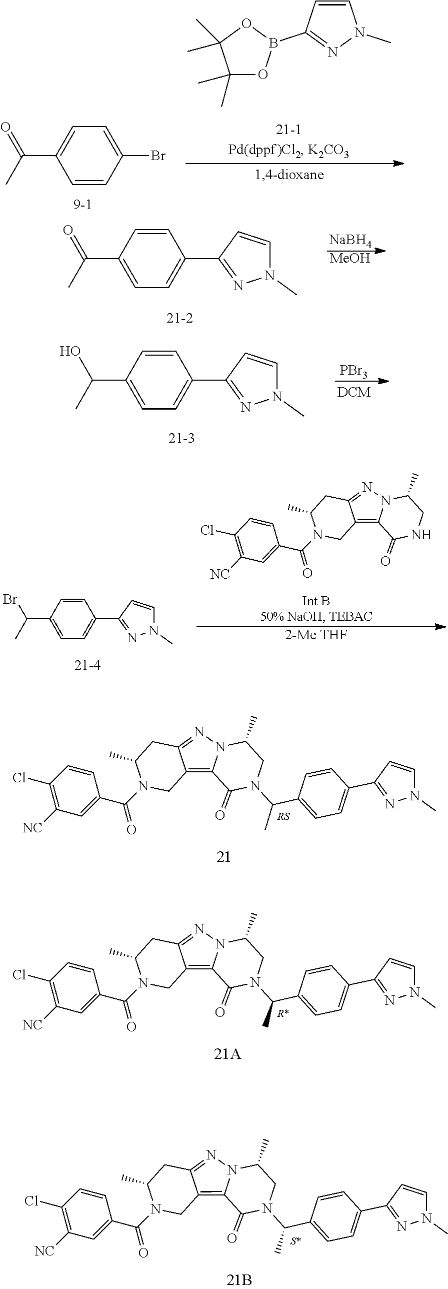

### Intermediate 21-2

### 1-(4-(1-Methyl-1H-pyrazol-3-yl)phenyl)ethanone

To a solution of 1-methyl-3-(4,4,5,5-tetramethyl-1,3,2-dioxaborolan-2-yl)-1H-pyrazole 21-1 (200 mg, 0.96 mmol), 1-(4-bromophenyl)ethanone 9-1 (230 mg, 1.16 mmol) and potassium carbonate (250 mg, 1.81 mmol) in 1,4-dioxane (3 mL) and water (1.5 mL) was added [1,1′-bis(diphenylphosphino)ferrocene]dichloropalladium(II) (20 mg, 0.03 mmol) at 20° C. under nitrogen atmosphere. Then the reaction mixture was stirred at 85° C. for 13 hours. The reaction mixture was quenched with brine (10 mL), extracted with ethyl acetate (10 mL) for three times. The combined organic layers were washed with water (30 mL) and brine (30 mL), dried over Na₂SO₄₍ₛ₎, filtered. The filtrate was concentrated to get a residue, which was purified by C18 chromatography (acetonitrile:water (+0.02% ammonium bicarbonate)=40-60%) to give the title product (170 mg, 97% purity from LCMS, 85.7% yield) as colorless solids. LC-MS (ESI): R_{T}=1.40 min, mass calcd. for C₁₂H₁₂N₂O 200.1 m/z found 201.1 [M+H]⁺. ¹H NMR (400 MHz, CDCl3) δ 7.99 (d, J=8.4 Hz, 2H), 7.89 (d, J=8.0 Hz, 2H), 7.41 (d, J=2.0 Hz, 1H), 6.62 (d, J=8.0 Hz, 1H), 3.98 (s, 3H), 2.63 (s, 3H).

### Intermediate 21-3

### 1-(4-(1-Methyl-1H-pyrazol-3-yl)phenyl)ethanol

To a solution of 1-(4-(1-methyl-1H-pyrazol-3-yl)phenyl)ethanone 21-2 (170 mg, 0.82 mmol) in methanol (2 mL) was added sodium borohydride (30 mg, 0.79 mmol) at 0° C., then the mixture was stirred at room temperature for 2 hours. The solution was quenched with water (10 mL) and extracted with ethyl acetate (20 mL) for three times. The combined organic layers were washed with water (30 mL) and brine (10 mL), dried over Na₂SO₄₍ₛ₎ and filtered. The filtrate was concentrated to give the title product (160 mg, 96% purity from LCMS, 92.2% yield) as colorless solids. LC-MS (ESI): R_{T}=1.32 min, mass calcd. for C₁₂H₁₄N₂O 202.1 m/z found 203.1 [M+H]⁺.

### Intermediate 21-4

### 3-(4-(1-Bromoethyl)phenyl)-1-methyl-1H-pyrazole

To a solution of 1-(4-(1-methyl-1H-pyrazol-3-yl)phenyl)ethanol 21-3 (60 mg, 96% purity, 0.29 mmol) in dichloromethane (1 mL) was added dropwise tribromophosphine (60 mg, 0.22 mmol) in dichloromethane (1 mL) at 0° C. The resulting mixture was stirred at 0° C. for 3 hours. The reaction mixture was poured into water (10 mL), extracted with dichloromethane (10 mL) for three times. The combined organic layers were washed with brine (10 mL), dried over Na₂SO₄₍ₛ₎, and filtered. The filtrate was concentrated in vacuum to give the title product (60 mg, 90% purity from ¹H NMR, 71.5% yield) as white solids. ¹H NMR (300 MHz, CDCl₃) δ 7.86 (d, J=8.1 Hz, 2H), 7.54-7.46 (m, 3H), 6.62 (d, J=2.4 Hz, 1H), 5.35-5.28 (m, 1H), 4.08 (s, 3H), 2.12 (d, J=7.2 Hz, 3H).

### Compound 21

### 2-Chloro-5-((3R,7R)-3,7-dimethyl-9-(1-(4-(1-methyl-1H-pyrazol-3-yl)phenyl)ethyl)-10-oxo-1,2,3,4,7,8,9,10-octahydropyrido[4′,3′:3,4]pyrazolo[1,5-a]pyrazine-2-carbonyl)benzonitrile

To a solution of 2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (150 mg, 100% yield, 0.39 mmol) and 3-(4-(1-bromoethyl)phenyl)-1-methyl-1H-pyrazole 21-4 (150 mg, 90% purity, 0.51 mmol) in 2-methyltetrahydrofuran (2 mL) were added 50% wt. sodium hydroxide solution (1 mL) and N-benzyl-N,N-diethylethanaminium chloride (10 mg, 0.11 mmol) at 25° C. The reaction mixture was stirred at 50° C. for 2 hours, then poured into water (10 mL), extracted with acetate (10 mL) for three times. The combined organic layers were washed with brine (10 mL), dried over Na₂SO₄₍ₛ₎, and filtered. The filtrate was and concentrated in vacuum, which was purified by C18 chromatography (acetonitrile:water (+0.02% ammonium bicarbonate)=40-60%) to give the title compound (170 mg, 96% purity from LCMS, 73.5% yield) as colorless solids. LC-MS (ESI): R_{T}=1.60 min, mass calcd. for C₃₁H₃₀ClN₇O₂ 567.2 m/z found 568.3 [M+H]⁺.

### Compounds 21A and 21B

### 2-Chloro-5-((3R,7R)-3,7-dimethyl-9-((R*)-1-(4-(1-methyl-1H-pyrazol-3-yl)phenyl)ethyl)-10-oxo-1,2,3,4,7,8,9,10-octahydropyrido[4′,3′:3,4]pyrazolo[1,5-a]pyrazine-2-carbonyl)benzonitrile (21A) and 2-Chloro-5-((3R,7R)-3,7-dimethyl-9-((S*)-1-(4-(1-methyl-1H-pyrazol-3-yl)phenyl)ethyl)-10-oxo-1,2,3,4,7,8,9,10-octahydropyrido[4′,3′:3,4]pyrazolo[1,5-a]pyrazine-2-carbonyl)benzonitrile (21B)

The racemate 2-chloro-5-((3R,7R)-3,7-dimethyl-9-(1-(4-(1-methyl-IH-pyrazol-3-yl)phenyl)ethyl)-10-oxo-1,2,3,4,7,8,9,10-octahydropyrido[4′,3′:3,4]pyrazolo[1,5-a]pyrazine-2-carbonyl)benzonitrile 21 (170 mg, 96% purity, 0.29 mmol) was separated by chiral prep HPLC (separation condition: Column: Chiralpak IC 5 μm 20*250 mm; Mobile Phase: ACN:IPA=90:10 at 25 mL/min; Temp: 30° C.; Wavelength: 254 nm) to give the title compounds 21A (51.4 mg, 99.4% purity, 31.3% yield, 100% stereopure) and 21B (53.4 mg, 99.5% purity, 32.6% yield, 99.9% stereopure) as white solids.

### 21A:

LC-MS (ESI): R_{T}=3.443 min, mass calcd. for C₃₁H₃₀ClN₇O₂ 567.2 m/z found 568.2 [M+H]⁺. Chiral analysis (Column: Chiralpak IC 5 μm 4.6*250 mm; Mobile Phase: ACN:IPA=90:10 at 1 mL/min; Temp: 30° C.; Wavelength: 254 nm, R_{T}=5.993 min). ¹H NMR (400 MHz, DMSO-d₆) δ 8.20 (s, 1H), 7.85-7.77 (m, 4H), 7.72 (d, J=1.6 Hz, 1H), 7.47-7.31 (m, 2H), 6.67 (d, J=2.0 Hz, 1H), 5.98-5.69 (m, 1H), 5.59-5.11 (m, 1H), 4.67-4.32 (m, 2H), 4.26-4.01 (m, 1H), 3.87 (s, 3H), 3.81-3.68 (m, 1H), 3.08-2.86 (m, 2H), 2.66-2.51 (m, 1H), 1.67-1.46 (m, 3H), 1.31-1.03 (m, 6H).

### 21B:

LC-MS (ESI): R_{T}=3.508 min, mass calcd. for C₃₁H₃₀ClN₇O₂ 567.2 m/z found 568.2 [M+H]⁺. Chiral analysis (Column: Chiralpak IC 5 μm 4.6*250 mm; Mobile Phase: ACN:IPA=90:10 at 1 mL/min; Temp: 30° C.; Wavelength: 254 nm, R_{T}=8.065 min). ¹H NMR (400 MHz, DMSO-d₆) δ 8.12 (s, 1H), 7.87-7.76 (m, 4H), 7.72 (d, J=2.0 Hz, 1H), 7.45-7.25 (m, 2H), 6.66 (s, 1H), 5.97-5.67 (m, 1H), 5.57-5.13 (m, 1H), 4.67-4.26 (m, 2H), 4.24-4.05 (m, 1H), 3.87 (s, 3H), 3.52-3.40 (m, 1H), 3.40-3.31 (m, 1H), 3.03-2.86 (m, 1H), 2.60-2.51 (m, 1H), 1.65-1.48 (m, 3H), 1.42 (d, J=5.4 Hz, 3H), 1.29-1.04 (m, 3H).

### Compounds 22A and 22B

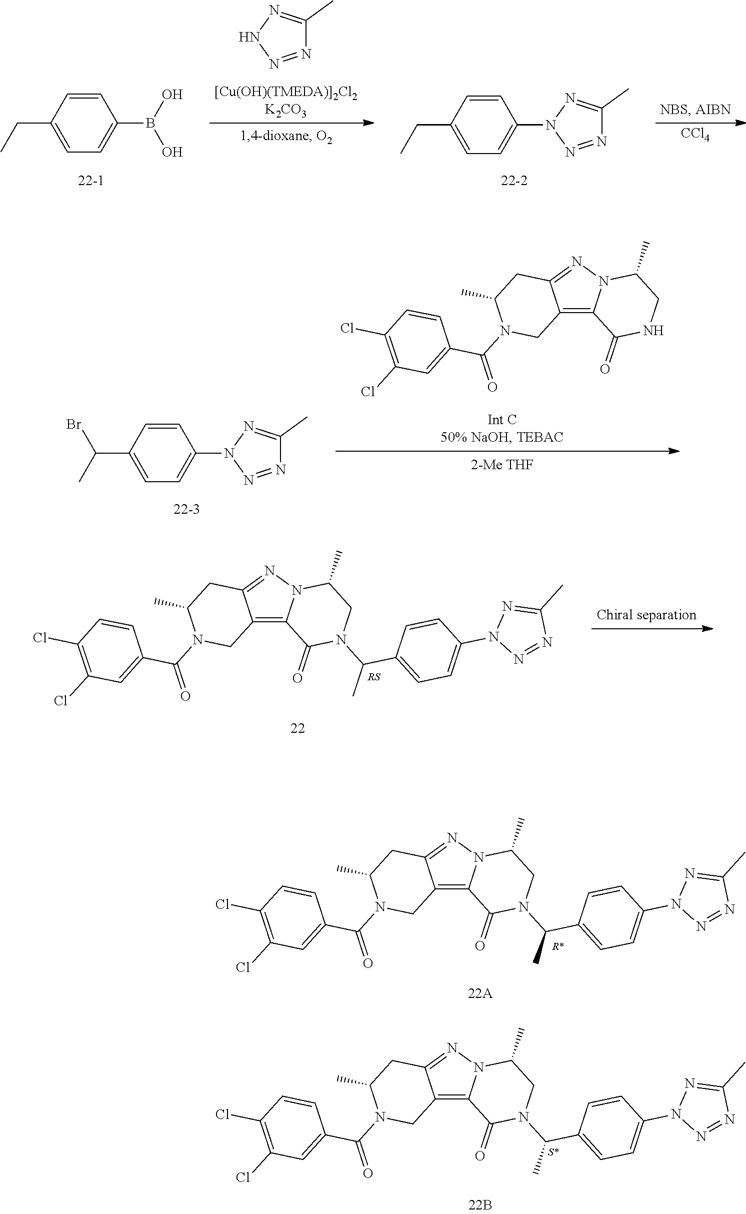

### Intermediate 22-2

### 2-(4-Ethylphenyl)-5-methyl-2H-tetrazole

To a solution of 5-methyltetrazole (1.0 g, 11.9 mmol) in 1,4-dioxane (30 mL), potassium carbonate (6.0 g, 43.4 mmol), Cu-TMEDA Catalyst (1.2 g, 2.58 mmol) was added (4-ethylphenyl) boronic acid 22-1 (3.0 g, 20.0 mmol). The mixture was stirred under an oxygen atmosphere at room temperature for 16 hours. The reaction mixture was quenched with water (50 mL) and extracted with ethyl acetate (50 mL) twice. The combined organic layers were washed with saturated sodium bicarbonate aqueous solution (50 mL), brine (100 mL), dried over Na₂SO₄₍ₛ₎ and filtered. The filtrate was concentrated to give a residue, which was purified by silica gel column chromatography (petroleum ether:ethyl acetate=10:1) to give the title compound (300 mg, 90% purity from ¹H NMR, 7.1% yield) as brown oil. ¹H NMR (400 MHz, CDCl₃) δ 8.00 (d, J=8.4 Hz, 2H), 7.36 (d, J=8.8 Hz, 2H), 2.74 (q, J=14.8 Hz, 2H), 2.63 (s, 3H), 1.28 (t, J=7.6 Hz, 3H).

### Intermediate 22-3

### 2-(4-(1-Bromoethyl) phenyl)-5-methyl-211-tetrazole

2-(4-ethylphenyl)-5-methyl-2H-tetrazole 22-2 (300 mg, 90% purity, 1.43 mmol), N-Bromo succinimide (375 mg, 2.11 mmol) and 2,2′-azobis(2-methylpropionitrile) (100 mg, 0.609 mmol) were mixed in perchloromethane (15 mL) at room temperature. After stirred at 90° C. for 32 hours, the reaction mixture was concentrated and purified by silica gel column chromatography (petroleum ether:ethyl acetate=10:1) to give the title crude compound which was further purified by C18 column (acetonitrile:water=50% to 60%) to give the title compound (130 mg, 90% purity from ¹H NMR, 30.5% yield) as brown oil. ¹H NMR (400 MHz, CDCl₃) δ 8.08 (d, J=8.8 Hz, 2H), 7.56 (d, J=8.4 Hz, 2H), 5.01 (q, J=12.8 Hz, 1H), 2.64 (s, 3H), 1.57 (d, J=6.0 Hz, 3H).

### Compound 22

### (3R,7R)-2-(3,4-Dichlorobenzoyl)-3,7-dimethyl-9-(1-(4-(5-methyl-2H-tetrazol-2-yl)phenyl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (150 mg, 100% purity, 0.381 mmol), 2-(4-(1-bromoethyl)phenyl)-5-methyl-2H-tetrazole 22-3 (70 mg, 90% purity, 0.23 mmol) in 2-methyltetrahydrofuran (10 mL) were added 50% wt. sodium hydroxide (5 mL) and benzyltriethylammonium chloride (30 mg, 0.13 mmol) at room temperature. After stirred at 60° C. under nitrogen atmosphere for 2 hours, the reaction mixture was quenched with water (50 mL) and extracted with ethyl acetate (50 mL) for three times. The combined organic layers were washed with brine (50 mL), dried over anhydrous Na₂SO₄₍ₛ₎ and filtrated, and the solvent was evaporated in vacuum to give a yellow residue, which was purified by C18 column (acetonitrile:water=50% to 60%) to give the title compound (150 mg, 91% purity from LCMS, 61.7% yield) as off-white solids. LC-MS (ESI): R_{T}=3.28 min, mass calcd. for C₂₈H₂₈Cl₂N₈O₂ 578.2 m/z found 579.4.

### Compounds 22A and 22B

### (3R,7R)-2-(3,4-dichlorobenzoyl)-3,7-dimethyl-9-((R*)-1-(4-(5-methyl-2H-tetrazol-2-yl)phenyl)ethyl)-1,2,3,4,8,9-hexahydropyrido[4′,3′:3,4]pyrazolo[1,5-a]pyrazin-10(7H)-one (22A) and (3R,7R)-2-(3,4-dichlorobenzoyl)-3,7-dimethyl-9-((S*)-1-(4-(5-methyl-2H-tetrazol-2-yl)phenyl)ethyl)-1,2,3,4,8,9-hexahydropyrido[4′,3′:3,4]pyrazolo[1,5-a]pyrazin-10(7H)-one (22B)

A racemic mixture of (3R,7R)-2-(3,4-dichlorobenzoyl)-3,7-dimethyl-9-(1-(4-(5-methyl-2H-tetrazol-2-yl)phenyl)ethyl)-1,2,3,4,8,9-hexahydropyrido[4′,3′:3,4]pyrazolo [1,5-a]pyrazin-10 (7H)-one 22 (150 mg, 91% purity, 0.236 mmol) was separated by chiral prep HPLC (separation condition: Column: Chiralpak IE 5 μm 20*250 mm; Mobile Phase: ACN:IPA=70:30 at 30 mL/min; Temp: 30° C.; Wavelength: 254 nm)), then further purified with C18 column (acetonitrile:water=50% to 70%) to give the title compounds 22A (37.1 mg, 99.5% purity from LCMS, 77.7% yield, 100% stereopure) as white solids and 22B (38.5 mg, 99.3% purity from LCMS, 80.5% yield, 99.9% stereopure) as white solids.

### 22A:

LC-MS (ESI): R_{T}=4.30 min, mass calcd. for C₂₈H₂₈Cl₂N₈O₂, 578.2 m/z found 579.4. Chiral analysis (Column: Chiral Pak IC 5 μm 20*250 mm; Mobile Phase: ACN:IPA=70:30, at 1 mL/min; Temp: 30° C.; Wavelength: 254 nm, R_{T}=9.679 min)). ¹H NMR (400 MHz, CDCl₃) δ 8.11 (d, J=8.8 Hz, 2H), 7.60-7.50 (m, 4H), 7.28-7.24 (m, 1H), 6.25-6.06 (m, 1H), 5.86-5.34 (m, 1H), 4.91-4.26 (m, 3H), 3.74-3.57 (m, 1H), 3.07-2.93 (m, 2H), 2.80-2.64 (m, 4H), 1.63 (d, J=6.8 Hz, 3H), 1.27-1.25 (m, 6H).

### 22B:

LC-MS (ESI): R_{T}=4.33 min, mass calcd. for C₂₈H₂₈Cl₂N₈O₂, 578.2 m/z found 579.4. Chiral analysis (Column: Chiral Pak IC 5 μm 20*250 mm; Mobile Phase: ACN:IPA=70:30, at 1 mL/min; Temp: 30° C.; Wavelength: 254 nm, R_{T}=11.260 min)). ¹H NMR (400 MHz, CDCl₃) δ 8.09 (d, J=8.8 Hz, 2H), 7.55-7.51 (m, 4H), 7.29 (d, J=2.0 Hz, 1H), 6.24-6.04 (m, 1H), 5.86-5.03 (m, 1H), 4.80-4.28 (m, 3H), 3.37-3.25 (m, 2H), 3.15-2.92 (m, 1H), 2.70-2.64 (m, 4H), 1.65 (d, J=7.2 Hz, 3H), 1.54 (d, J=6.8 Hz, 3H), 1.32-1.25 (m, 3H).

### Compounds 23A and 23B

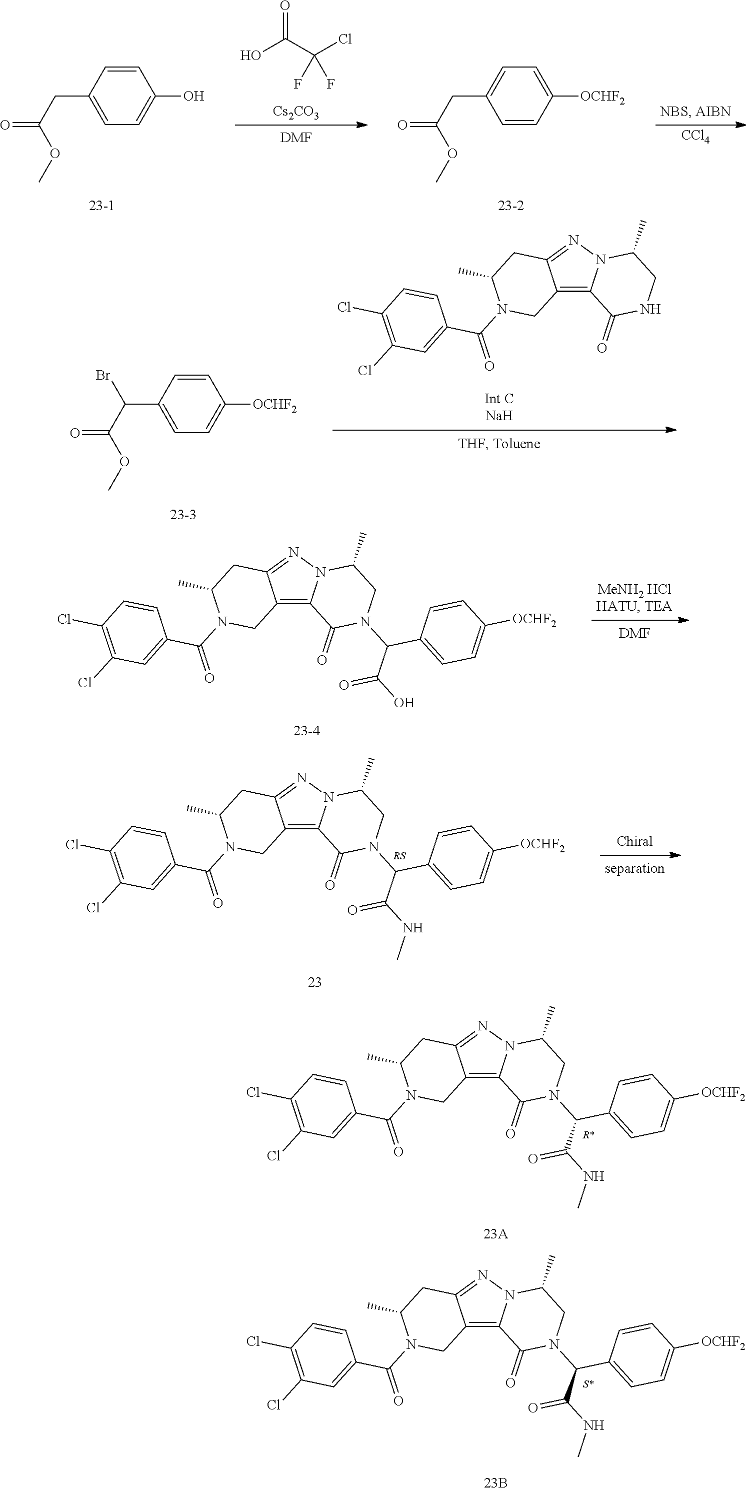

### Intermediate 23-2

### Methyl 2-(4-(difluoromethoxy)phenyl)acetate

Cesium carbonate (20 g, 61.3 mmol) was added to a solution of methyl 2-(4-hydroxyphenyl) acetate 23-1 (5.0 g, 30.0 mmol) and sodium chlorodifluoroacetate (9.0 g, 59.0 mmol) in N, N-dimethylformamide (50 mL). The reaction mixture was stirred at 80° C. for 3 hours. After cooled to room temperature, the reaction mixture was added ethyl acetate (200 mL) and washed with water (200 mL) twice, brine (200 mL), dried over Na₂SO₄₍ₛ₎ and filtered. The filtrate was concentrated and purified by C18 column (acetonitrile:water (+0.02% ammonium acetate)=5% to 100%) to give the title compound (2.5 g, 95% purity from ¹H NMR, 36.5% yield) as brown oil. ¹H NMR (400 MHz, CDCl₃) δ 7.29-7.27 (m, 2H), 7.09-7.07 (m, 2H), 6.49 (t, J=74.0 Hz, 1H), 3.70 (s, 3H), 3.61 (s, 2H).

### Intermediate 23-3

### Methyl 2-bromo-2-(4-(difluoromethoxy) phenyl)acetate

To a solution of methyl 2-(4-(difluoromethoxy)phenyl)acetate 23-2 (2.0 g, 95% purity, 8.79 mmol), N-bromosuccinimide (2.0 g, 11.2 mmol) in carbon tetrachloride (50 mL) was added 2,2′-azobis(2-methylpropionitrile) (400 mg, 2.436 mmol). The reaction mixture was stirred at 70° C. for 3 hours. After cooled to room temperature, the reaction mixture was concentrated and purified by silica gel column chromatography (petroleum ether:ethyl acetate=10:1) to give the title compound (1.3 g, 95% purity from ¹H NMR, 47.6% yield) as brown oil. ¹H NMR (400 MHz, CDCl₃) δ 7.57 (d, J=8.4 Hz, 2H), 7.57 (d, J=8.8 Hz, 2H), 6.52 (t, J=73.6 Hz, 1H), 5.34 (s, 1H), 3.80 (s, 3H).

### Intermediate 23-4

### 2-((3R,7R)-2-(3,4-Dichlorobenzoyl)-3,7-dimethyl-10-oxo-1,2,3,4,7,8-hexahydro pyrido[4′,3′:3,4]pyrazolo[1,5-a]pyrazin-9(10H)-yl)-2-(4-(difluoromethoxy)phenyl)acetic acid

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (300 mg, 100% purity, 0.763 mmol) in toluene (3 mL) and tetrahydrofuran (3 mL) was added 60% sodium hydride dispersion in mineral oil (100 mg, 2.50 mmol) at 0° C. Then methyl 2-bromo-2-(4-(difluoromethoxy)phenyl)acetate 23-3 (400 mg, 95% purity, 1.29 mmol) was added. After stirred at 60° C. under nitrogen atmosphere for 16 hours, the reaction mixture was quenched with water (20 mL) and extracted with ethyl acetate (20 mL) for three times. The combined organic layers were washed with brine (20 mL), dried over Na₂SO₄₍ₛ₎ and filtrated, and the solvent was evaporated in vacuum to give the title compound (250 mg, 91% purity from LCMS, 50.2% yield) as yellow oil. LC-MS (ESI): R_{T}=1.37 min, mass calcd. for C₂₇H₂₄Cl₂F₂N₄O₅ 593.3 m/z found 594.3.

### Compound 23

### 2-((3R,7R)-2-(3,4-dichlorobenzoyl)-3,7-dimethyl-10-oxo-1,2,3,4,7,8-hexahydro pyrido[4′,3′:3,4]pyrazolo[1,5-a]pyrazin-9(10H)-yl)-2-(4-(difluoromethoxy) phenyl)-N-methylacetamide

To a solution of 2-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)-2-(4-(difluoromethoxy) phenyl)acetic acid 23-4 (250 mg, 91% purity, 0.383 mmol), methanamine hydrochloride (250 mg, 3.70 mmol) in N,N-dimethylformamide (2 mL) were added triethylamine (500 mg, 4.94 mmol) and 2-(7-Azabenzotriazol-1-yl)-N,N,N′,N′-tetramethyluronium hexafluorophosphate (300 mg, 0.789 mmol) at 0° C. After stirred at 30° C. under nitrogen atmosphere for 2 hours, the reaction mixture was quenched with water (25 mL) and extracted with ethyl acetate (20 mL) for three times. The combined organic layers were washed with brine (50 mL) and dried over Na₂SO₄₍ₛ₎ and filtered. The filtrate was concentrated in vacuum to give a residue, which was purified by C18 column (acetonitrile:water=50% to 60%) to give the title compound (150 mg, 100% purity from LCMS, 64.5% yield) as off-white solids. LC-MS (ESI): R_{T}=1.37 min, mass calcd. for C₂₇H₂₄Cl₂F₂N₄O₅ 593.3 m/z found 594.3.

### Compounds 23A and 23B

### (R*)-2-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)-2-(4-(difluoromethoxy)phenyl)-N-methylacetamide (23A) and (S*)-2-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)-2-(4-(difluoromethoxy)phenyl)-N-methylacetamide (23B)

A racemic mixture of 2-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)-2-(4-(difluoromethoxy)phenyl)-N-methylacetamide 23 (150 mg, 100% purity, 0.247 mmol) was separated by chiral prep HPLC (separation condition: Column: Chiralpak IC 5 μm 20*250 mm; Mobile Phase: ACN:IPA=70:30 at 30 mL/min; Temp: 35° C.; Wavelength: 254 nm)), then further purified with C18 column (acetonitrile:water=50% to 70%) to give the title compounds 23A (23 mg, 97.8% purity, 75.5% yield, 100% stereopure) as white solids and 23B (58 mg, 99.7% purity, 86.9% yield, 99.9% stereopure) as white solids.

### 23A:

LC-MS (ESI): R_{T}=3.184 min, mass calcd. for C₂₈H₂₇Cl₂F₂N₅O₄ 605.2 m/z found 606.4. Chiral analysis (Column: Chiral Pak IC 5 m 4.6*250 mm; Mobile Phase: ACN:IPA=70:30 at 1 mL/min; Temp: 30° C.; Wavelength: 254 nm, R_{T}=3.628 min). ¹H NMR (400 MHz, CDCl₃) δ 7.53-7.50 (m, 2H), 7.43-7.49 (m, 2H), 7.25-7.23 (m, 1H), 7.16 (d, J=8.4 Hz, 2H), 6.54 (t, J=72.8 Hz, 1H), 6.30-4.89 (m, 3H), 5.07-4.18 (m, 3H), 4.00-3.72 (m, 1H), 3.39-3.36 (m, 1H), 3.06 (br s, 1H), 2.88 (d, J=4.8 Hz, 3H), 2.71-2.66 (m, 1H), 1.56 (d, J=6.8 Hz, 3H), 1.25 (s, 3H). ¹⁹F NMR (376 MHz, CDCl₃) δ −81.37.

### 23B:

LC-MS (ESI): R_{T}=3.189 min, mass calcd. for C₂₈H₂₇Cl₂F₂N₅O₄ 605.2 m/z found 606.2 Chiral analysis (Column: Chiral Pak IC 5 m 4.6*250 mm; Mobile Phase: ACN:IPA=70:30 at 1 mL/min; Temp: 30° C.; Wavelength: 254 nm, R_{T}=6.874 min)). ¹H NMR (400 MHz, CDCl₃) δ 7.54-7.48 (m, 2H), 7.43-7.37 (m, 2H), 7.25-7.23 (m, 1H), 7.16 (d, J=8.4 Hz, 2H), 6.54 (t, J=72.8 Hz, 1H), 6.33-6.24 (m, 1H), 6.00-5.36 (m, 2H), 4.83-4.26 (m, 3H), 4.10-4.07 (m, 2H), 3.18-3.03 (m, 1H), 2.87 (d, J=4.0 Hz, 3H), 2.68-2.64 (m, 1H), 1.35 (d, J=6.8 Hz, 3H), 1.25 (s, 3H). ¹⁹F NMR (376 MHz, CDCl₃) δ −81.42.

### Compounds 24A and 24B

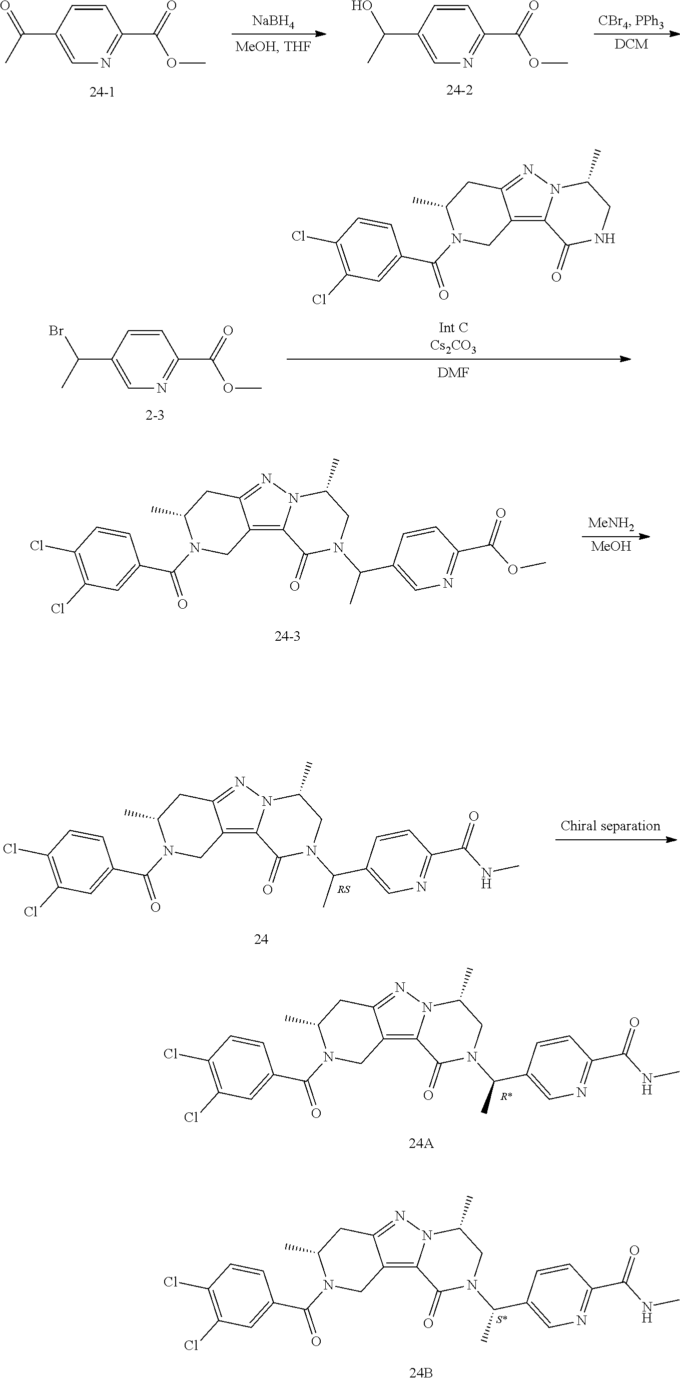

### Intermediate 24-2

### Methyl 5-(1-hydroxyethyl)picolinate

To a solution of methyl 5-acetylpicolinate 24-1 (1.80 g, 90% purity, 9.04 mmol) in tetrahydrofuran (20 mL) and methanol (2 mL) was added sodium tetrahydroborate (1.03 g, 27.1) slowly at 0° C. After stirred at room temperature for 3 hours, the reaction mixture was diluted with water (10 mL) and extracted with ethyl acetate (10 mL) for three times. The combined organic layers were dried over Na₂SO₄₍ₛ₎ and filtered. The filtrate was concentrated in vacuum to give the residue, which was purified by silica gel column chromatography (petroleum ether:ethyl acetate=6:1 to 2:1) to give the title compound (1.20 g, 100% purity from LCMS, 73% yield) as yellow oil. LC-MS (ESI): R_{T}=0.74 min, mass calcd. for C₉H₁₁NO₃ 181.1, m/z found 182.0 [M+H]⁺.

### Intermediate 24-3

### Methyl 5-(1-bromoethyl)picolinate

To a solution of methyl 5-(1-hydroxyethyl)picolinate 24-2 (500 mg, 100% purity, 2.76 mmol), triphenylphosphine (981 mg, 3.74 mmol) in dichloromethane (5 mL) was added perbromomethane (1.07 g, 3.23 mmol) slowly at 0° C. After stirred at room temperature for 5 hours, the reaction mixture was concentrated in vacuum to give the residue, which was purified by silica gel column chromatography (petroleum ether:ethyl acetate=10:1 to 5:1) to give the title compound (310 mg, 86% purity from LCMS, 44% yield) as brown oil. LC-MS (ESI): R_{T}=1.40 min, mass calcd. for C₉H₁₀BrNO₂ 243.0, m/z found 244.0 [M+H]⁺.

### Intermediate 24-3

### Methyl 5-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)picolinate

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (305 mg, 95% purity, 0.737 mmol) in N,N-dimethylformamide (18 mL) were added methyl 5-(1-bromoethyl)picolinate 2-3 (250 mg, 86% purity, 0.881 mmol) and cesium carbonate (720 mg, 2.21 mmol) at room temperature. After heated at 80° C. for 16 hours. The reaction mixture was diluted with water (20 mL) and extracted with ethyl acetate (50 mL) twice. The combined organic layers were washed with water (100 mL) for three times and brine (100 mL), dried over Na₂SO₄₍ₛ₎ and filtered. The filtrate was concentrated in vacuum to give the residue, which was purified by silica gel column chromatography (petroleum ether:ethyl acetate=6:1 to 2:1) to give the title compound (320 mg, 77% purity for LCMS, 60% yield) as yellow solids. LC-MS (ESI): R_{T}=1.58 min, mass calcd. for C₂₇H₂₇Cl₂N₅O₄ 555.1, m/z found 556.1 [M+H]⁺.

### Compound 24

### 5-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylpicolinamide

To a solution of the methyl 5-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)picolinate 24-3 (300 mg, 77% purity, 0.415 mmol) in methanol (3 mL) was added 2 M methanamine tetrahydrofuran (1.2 mL, 2.40 mmol) in a microwave tube at room temperature. After heated at 80° C. for 16 hours. The reaction mixture was concentrated in vacuum to give a residue, which was purified by silica gel column chromatography (petroleum ether:ethyl acetate=5:1 to 2:1) to give the title compound (160 mg, 83% purity for LCMS, 58% yield) as yellow solids. LC-MS (ESI): R_{T}=1.55 min, mass calcd. for C₂₇H₂₈Cl₂N₆O₃ 554.2, m/z found 555.1 [M+H]⁺.

### Compounds 24A and 24B

### 5-((R*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylpicolinamide (24A) and 5-((S*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)-N-methylpicolinamide (24B)

A racemic mixture of 5-(1-((3R,7R)-2-(3,4-dichlorobenzoyl)-3,7-dimethyl-10-oxo-1,2,3,4,7,8-hexahydropyrido[4′,3′:3,4]pyrazolo[1,5-a]pyrazin-9(10H)-yl)ethyl)-N-methylpicolinamide 24 (160 mg, 90% purity, 0.259 mmol) was separated by Chiral Prep-HPLC (Column: Chiralpak IE 10 μm 30 mm*250 mm; Mobile Phase: ACN:IPA=50:50 at 25 mL/min; Col. Temp: 30° C.; Wavelength: 214 nm), then further purified with Prep. HPLC (Column: Gilson Xbridge C18 (5 μm 19*150 mm), Mobile Phase A: Water (0.1% ammonium bicarbonate), Mobile Phase B: acetonitrile, UV: 254 nm, Flow rate: 15 mL/min, Gradient: 20-70% (% B)) to afford the title compounds 24A (50 mg, 99.3% purity from LCMS, 34% yield, 99.9% stereopure) as white solids and 24B (60 mg, 99.4% purity from LCMS, 41% yield, 99.7% stereopure) as white solids.

### 24A:

LC-MS (ESI): R_{T}=3.085 min, mass calcd. for C₂₇H₂₈Cl₂N₆O₃ 554.2, m/z found 555.2 [M+H]⁺. Chiral analysis (Column: Chiralpak IE 5 μm 4.6*250 mm; Mobile Phase: ACN:IPA=50:50 at 25 mL/min; Temp: 30° C.; Wavelength: 254 nm, R_{T}=8.385 min). ¹H NMR (400 MHz, DMSO-d₆) δ 8.72 (d, J=3.6 Hz, 1H), 8.63 (s, 1H), 8.07-7.94 (m, 2H), 7.79-7.72 (m, 2H), 7.45-7.43 (m, 1H), 5.97-5.86 (m, 1H), 5.54-5.21 (m, 1H), 4.62-4.37 (m, 2H), 4.25-4.07 (m, 1H), 3.88-3.77 (m, 1H), 3.29 (s, 1H), 3.19-3.08 (m, 1H), 2.96-2.88 (m, 1H), 2.82 (d, J=4.8 Hz, 3H), 1.63 (s, 3H), 1.24 (d, J=6.4 Hz, 3H), 1.19-1.06 (m, 3H).

### 24B:

LC-MS (ESI): R_{T}=4.118 min, mass calcd. for C₂₇H₂₈Cl₂N₆O₃ 554.2, m/z found 555.2 [M+H]⁺. Chiral analysis (Column: Chiralpak IE 5 μm 4.6*250 mm; Mobile Phase: ACN:IPA=50:50 at 25 mL/min; Temp: 30° C.; Wavelength: 254 nm, R_{T}=12.710 min). ¹H NMR (400 MHz, DMSO-d₆) δ 8.71 (s, 1H), 8.61 (s, 1H), 8.06-7.89 (m, 2H), 7.75-7.73 (m, 2H), 7.45-7.43 (m, 1H), 6.02-5.75 (m, 1H), 5.55-5.23 (m, 1H), 4.61-4.33 (m, 2H), 4.25-4.07 (m, 1H), 3.54-3.44 (m, 2H), 3.29-3.25 (m, 1H), 2.95-2.90 (m, 1H), 2.81 (d, J=4.8 Hz, 3H), 1.62 (s, 3H), 1.44 (d, J=6.4 Hz, 3H), 1.23-1.06 (m, 3H).

### Compounds 25A and 25B

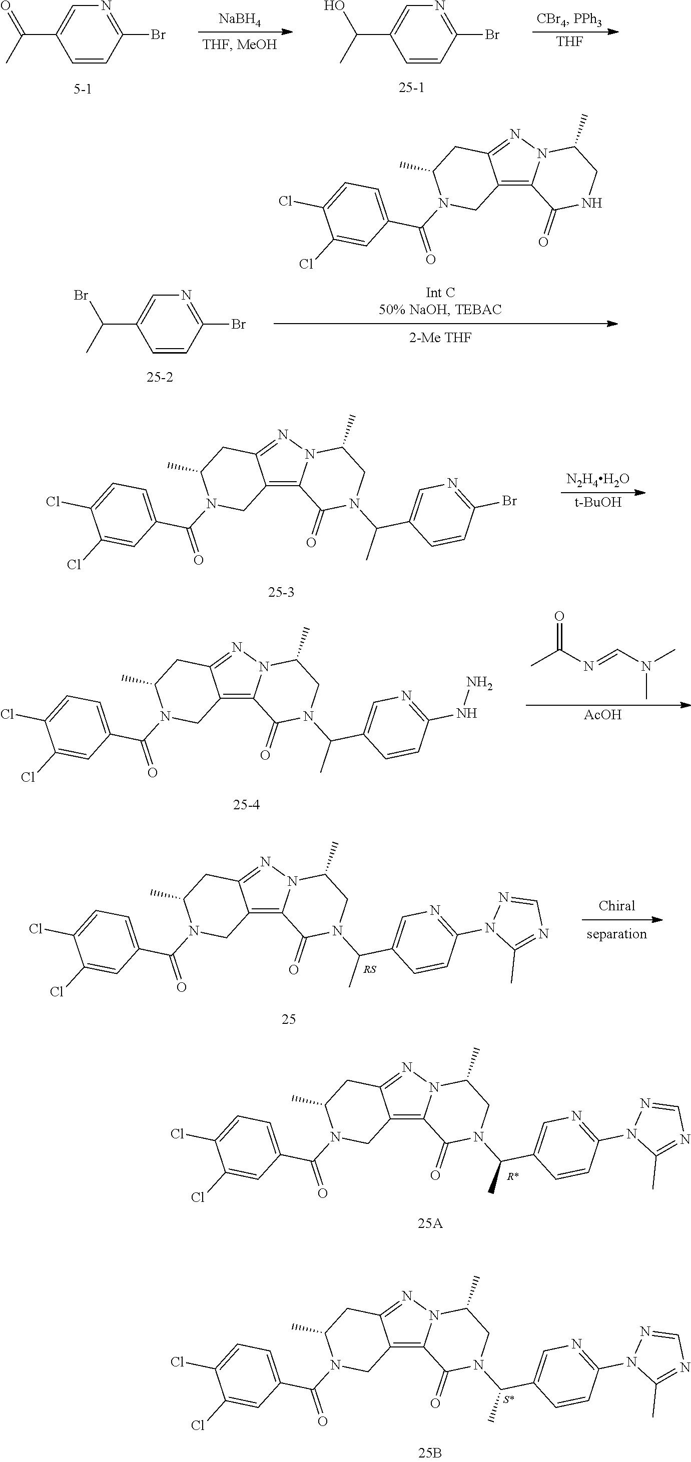

### Intermediate 25-1

### 2-(6-Bromopyridin-3-yl)ethano

To a solution of 1-(6-bromopyridin-3-yl)ethan-1-one 5-1 (1.0 g, 5.00 mmol) in tetrahydrofuran (10 mL) and methanol (1 mL) was added sodium tetrahydroborate (100 mg, 2.64 mmol). After stirred at 25° C. for 1 hour, the reaction mixture was quenched with water, extracted with ethyl acetate (100 mL) for three times. The combined organic layers were washed with brine (100 mL), dried over Na₂SO₄(1), filtered. The filtrate was concentrated to give the title compound (1.1 g, 87% purity from LCMS, 94.7% yield) as yellow oil. LC-MS (ESI): R_{T}=1.27 min, mass calcd. for C₇H₈BrNO 201.0, m/z found 201.9 [M+H]⁺.

### Intermediate 25-2

### 2-Bromo-5-(I-bromoethyl)pyridine

To a solution of 1-(6-bromopyridin-3-yl)ethanol 25-1 (1.1 g, 87% purity, 4.74 mmol) in dichlormethane (10 mL) was added perbromomethane (2.3 μg, 6.94 mmol) and triphenylphosphine (1.8 g, 6.86 mmol) at 0° C. After stirred at room temperature for 2 hours, the reaction mixture was quenched with saturated sodium bicarbonate solution (100 mL), extracted with dichloroethane (100 mL) twice. The organic layers were washed with brine (100 mL), dried over Na₂SO₄₍ₛ₎, filtered. The filtrate was concentrated and purified by silica gel column chromatography (petroleum ether:ethyl acetate=5:1) to give the title compound (1.3 g, 85% purity from LCMS, 88% yield) as a yellow oil. LC-MS (ESI): R_{T}=1.57 min, mass calcd. for C₇H₇Br₂N 264.9, m/z found 265.8 [M+H]⁺.

### Intermediate 25-3

### (3R,7R)-9-(1-(6-Bromopyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (600 mg, 100% purity, 1.53 mmol) and 2-bromo-5-(1-bromoethyl)pyridine 25-2 (700 mg, 100% purity, 2.64 mmol) in 2-methyltetrahydrofuran (3 mL) were added 50% sodium hydroxide in water (3 mL) and N-benzyl-N,N-diethylethanaminium chloride (30 mg, 0.132 mmol) at room temperature under nitrogen atmosphere. After stirred at room temperature under nitrogen atmosphere for 3 hours. The reaction mixture was added water (30 mL) and extracted with dichloromethane (20 mL) for three times. The combined organic layers were washed with brine (50 mL) then dried over Na₂SO₄₍ₛ₎ and filtered. The filtrate was concentrated and purified by C18 column (acetonitrile: water (0.1% ammonium bicarbonate)=5% to 95%) to give the title compound (700 mg, 90% purity from LCMS, 720% yield) as white solids. LC-MS (ESI): R_{T}=1.33 min, mass calcd. for C₂₅H₂₄BrCl₂N₅O₂ 575.0, m/z found 576.0 [M+H]⁺.

### Intermediate 25-4

### (3R,7R)-2-(3,4-Dichlorobenzoyl)-9-(1-(6-hydrazinylpyridin-3-yl)ethyl)-3,7-dimethyl-1,2,3,4,8,9-hexahydropyrido[4′,3′:3,4]pyrazolo[1,5-a]pyrazin-10(7H)-one

To the solution of (3R,7R)-9-(1-(6-bromopyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 25-3 (500 mg, 90% purity, 0.733 mmol) in butan-1-ol (5 mL) was added 85% hydrazine hydrate in water (5 mL) at room temperature under nitrogen atmosphere. The reaction mixture was cooled to room temperature after stirred at 130° C. for 6 hours, then the reaction mixture was purified by C18 column (acetonitrile:water (0.1% ammonium bicarbonate)=5% to 95%) to give the title compound (350 mg, 90% purity from LCMS, 81% yield) as light yellow oil. LC-MS (ESI): R_{T}=1.37 min, mass calcd. for C₂₅H₂₇Cl₂N₇O₂ 527.2, m/z found 528.1 [M+H]⁺.

### Compound 25

### (3R,7R)-2-(3,4-dichlorobenzoyl)-3,7-dimethyl-9-(1-(6-(5-methyl-11H-1,2,4-triazol-1-yl)pyridin-3-yl)ethyl)-1,2,3,4,8,9-hexahydropyrido[4′,3′:3,4]pyrazolo[1,5-a]pyrazin-10(7H)-one

To the solution of (3R,7R)-2-(3,4-dichlorobenzoyl)-9-(1-(6-hydrazinylpyridin-3-yl)ethyl)-3,7-dimethyl-1,2,3,4,8,9-hexahydropyrido[4′,3′:3,4]pyrazolo[1,5-a]pyrazin-10(7H)-one 25-4 (350 mg, 90% purity, 0.596 mmol) in acetic acid (5 mL) was added N-((dimethylamino)methylene)acetamide (180 mg, 80% purity, 1.26 mmol). After stirred at 90° C. for 6 hours, the reaction mixture was cooled to room temperature. Then water (30 mL) was added and extracted with dichloromethane (20 mL) for three times. The combined organic layers were washed with brine (50 mL) then dried over Na₂SO₄₍ₛ₎, concentrated and purified by C18 column (acetonitrile:water (0.1% ammonium bicarbonate)=5% to 95%) to give the title compound (200 mg, 100% purity from LCMS, 58% yield) as white solids. LC-MS (ESI): R_{T}=1.61 min, mass calcd. for C₂₈H₂₈Cl₂N₈O₂ 578.2, m/z found 579.1 [M+H]⁺.

### Compounds 25A and 25B

### (3R,7R)-2-(3,4-dichlorobenzoyl)-3,7-dimethyl-9-((R*)-1-(6-(5-methyl-11H-1,2,4-triazol-1-yl)pyridin-3-yl)ethyl)-1,2,3,4,8,9-hexahydropyrido[4′,3′:3,4]pyrazolo[1,5-a]pyrazin-10(7H)-one (25A) and (3R,7R)-2-(3,4-dichlorobenzoyl)-3,7-dimethyl-9-((S*)-1-(6-(5-methyl-11H-1,2,4-triazol-1-yl)pyridin-3-yl)ethyl)-1,2,3,4,8,9-hexahydropyrido[4′,3′:3,4]pyrazolo[1,5-a]pyrazin-10(7H)-one (25B)

A racemic mixture of (3R,7R)-2-(3,4-dichlorobenzoyl)-3,7-dimethyl-9-(1-(6-(5-methyl-1H-1,2,4-triazol-1-yl)pyridin-3-yl)ethyl)-1,2,3,4,8,9-hexahydropyrido[4′,3′:3,4]pyrazolo[1,5-a]pyrazin-10(7H)-one 25 (200 mg, 100% purity, 0.345 mmol) was separated by Chiral Prep-HPLC (Column: Chiralpak IA 5 μm 20 mm*250 mm; Mobile Phase: MeOH:DCM=70:30 at 20 mL/min; Col. Temp: 30° C.; Wavelength: 254 nm) to afford the title compounds 25A (80 mg, 99.3% purity from LCMS, 40% yield, 100% stereopure) as white solids and 25B (50 mg, 99.7% purity from LCMS, 25% yield, 99.8% stereopure) as white solids.

### 25A:

LC-MS (ESI): R_{T}=2.412 min, mass calcd. for C₂₈H₂₈Cl₂N₈O₂ 578.2, m/z found 579.2 [M+H]⁺. Chiral HPLC (Column: Chiralpak IA 5 m 4.6*250 mm; Mobile Phase: MeOH:DCM=70:30 at 1 mL/min; Temp: 30° C.; Wavelength: 254 nm, R_{T}=4.524 min). ¹H NMR (400 MHz, CDCl₃) δ 8.52 (s, 1H), 7.93-7.86 (m, 3H), 7.54-7.50 (m, 2H), 7.29-7.27 (m, 1H), 6.23-6.07 (m, 1H), 5.74-5.30 (m, 1H), 4.87-4.26 (m, 3H), 3.75-3.59 (m, 1H), 3.07-3.02 (m, 2H), 2.88 (s, 3H), 2.72-2.63 (m, 1H), 1.66 (d, J=6.8 Hz, 3H), 1.32 (d, J=6.8 Hz, 3H), 1.27 (d, J=4.8 Hz, 3H).

### 25B:

LC-MS (ESI): R_{T}=2.488 min, mass calcd. for C₂₈H₂₈Cl₂N₈O₂ 578.2, m/z found [M+H]⁺=579.2. Chiral HPLC (Column: Chiralpak IA 5 m 4.6*250 mm; Mobile Phase: MeOH:DCM=70:30 at 1 mL/min; Temp: 30° C.; Wavelength: 254 nm, R_{T}=5.793 min). ¹H NMR (400 MHz, CDCl₃) δ 8.49 (s, 1H), 7.91-7.89 (m, 2H), 7.84-7.79 (m, 1H), 7.54-7.50 (m, 2H), 7.31-7.28 (m, 1H), 6.25-6.05 (m, 1H), 5.81-5.40 (m, 1H), 4.87-4.33 (m, 3H), 3.42-3.28 (m, 2H), 3.12-3.00 (m, 1H), 2.88 (s, 3H), 2.72-2.65 (m, 1H), 1.67 (d, J=6.8 Hz, 3H), 1.56 (d, J=6.4 Hz, 3H), 1.26 (d, J=4.4 Hz, 3H).

### Compounds 26A, 26B, 27A and 27B

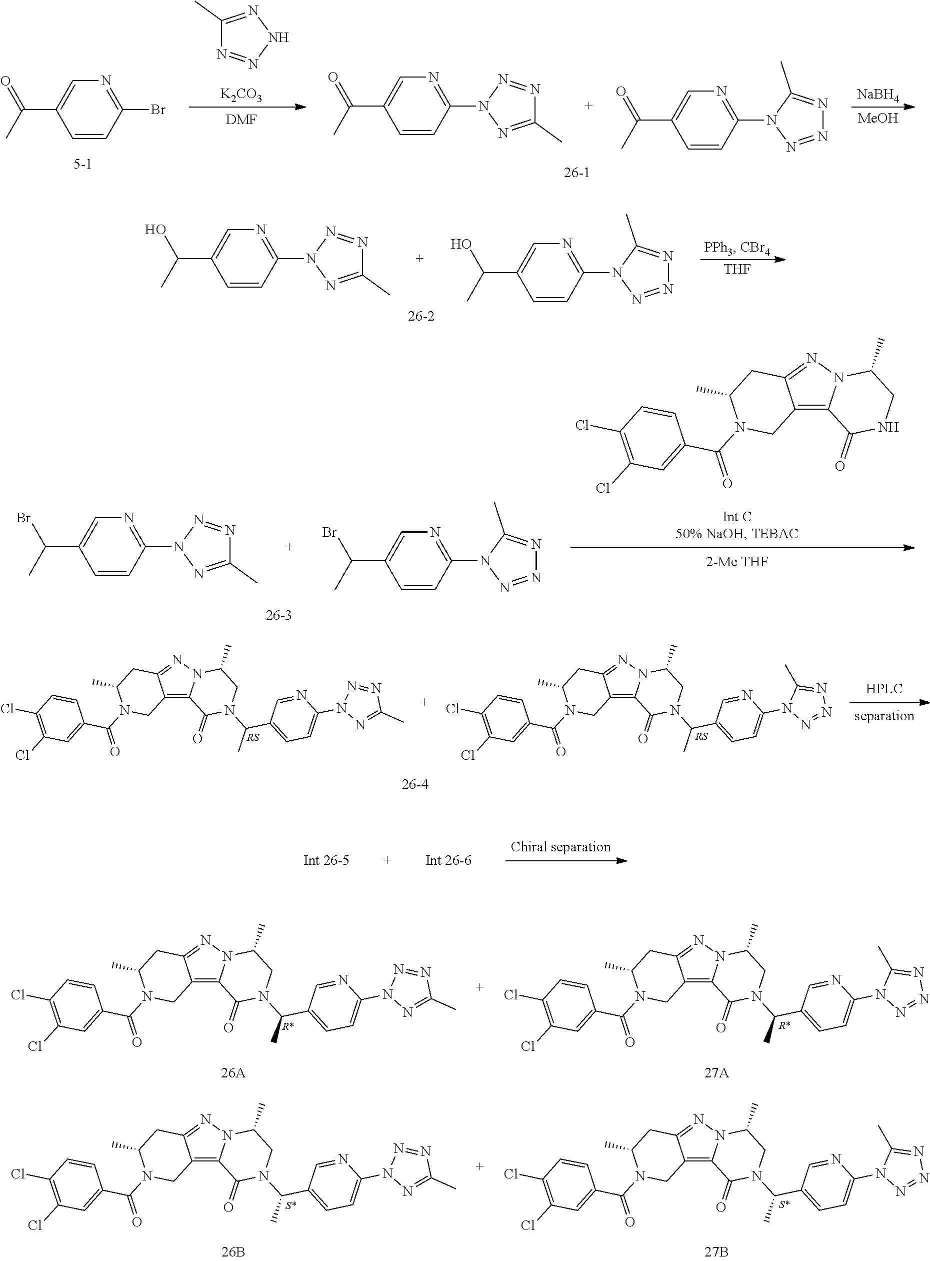

### Intermediate 26-1

### Mixture of 1-(6-(5-methyl-2H-tetrazol-2-yl)pyridin-3-yl)ethanone and 1-(6-(5-methyl-1H-tetrazol-1-yl)pyridin-3-yl)ethan-1-one

To the solution of 1-(6-bromopyridin-3-yl)ethan-1-one 5-1 (2.00 g, 10.0 mmol) in N,N-dimethylformamide (20 mL) was added 5-methyl-2H-tetrazole (1.70 g, 20.2 mmol) and potassium carbonate (4.24 g, 30.7 mmol). The reaction mixture was cooled to room temperature After stirred at 100° C. for 4 hours. Then the reaction mixture was diluted with water (100 mL) and extracted with ethyl acetate (100 mL) twice. The combined organic layers were washed with brine (100 mL) twice, dried over Na₂SO₄₍ₛ₎ and filtered. The filtrate was concentrated to get a residue, which was purified by column chromatography on silica gel (petroleum ether:ethyl acetate=10:1 to 3:1) to give the desired mixture (1.50 g, 90% purity from ¹H NMR, 66% yield) as yellow solids. LC-MS (ESI): R_{T}=1.30 min, mass calcd. for C₉H₉N₅O 203.1, m/z found 204.1 [M+H]⁺.

### Intermediate 26-2

### Mixture of 1-(6-(5-methyl-2H-tetrazol-2-yl)pyridin-3-yl)ethanol and 1-(6-(5-methyl-11H-tetrazol-1-yl)pyridin-3-yl)ethan-1-ol

To a solution of 1-(6-(5-methyl-2H-tetrazol-2-yl)pyridin-3-yl)ethanone and 1-(6-(5-methyl-1H-tetrazol-1-yl)pyridin-3-yl)ethan-1-one 26-1 (1.30 g, 90% purity, 5.76 mmol) in methanol (25 mL) was added sodium borohydride (180 mg, 4.76 mmol) at 0° C. After stirred at 0° C. for 1 hour, the mixture was quenched with saturated ammonium chloride aqueous solution (10 mL), then concentrated to get a residue, which was purified by column chromatography on silica gel (petroleum ether:ethyl acetate=10:1 to 3:1) to give the desired mixture (850 mg, 90% purity from ¹H NMR, 65% yield) as yellow oil. LC-MS (ESI): R_{T}=1.14 min & 1.18 min, mass calcd. for C₉H₁₁N₅O 205.1, m/z found 206.1 [M+H]⁺.

### Intermediate 26-3

### Mixture of 5-(1-bromoethyl)-2-(5-methyl-2H-tetrazol-2-yl)pyridine and 5-(1-bromoethyl)-2-(5-methyl-1H-tetrazol-1-yl)pyridine

To a solution of 1-(6-(5-methyl-2H-tetrazol-2-yl)pyridin-3-yl)ethanol and 1-(6-(5-methyl-1H-tetrazol-1-yl)pyridin-3-yl)ethan-1-ol 26-2 (800 mg, 90% purity, 3.51 mmol) in tetrahydrofuran (16 mL) were added triphenylphosphine (1.50 g, 1.72 mmol) and perbromomethane (1.50 g, 4.50 mmol) at 0° C. After stirred at 25° C. for 2 hours, the reaction mixture was concentrated to get a residue, which was purified by column chromatography on silica gel (petroleum ether:ethyl acetate=20:1 to 5:1) to give the desired compound (600 mg, 90% purity from ¹H NMR, 57% yield) as yellow oil. LC-MS (ESI): R_{T}=1.50 min, mass calcd. for C₉H₁₀BrN₅ 267.0, m/z found 268.0 [M+H]⁺.

### Intermediate 26-4

### (3R,7R)-2-(3,4-dichlorobenzoyl)-3,7-dimethyl-9-(1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-(1-(6-(5-methyl-11H-tetrazol-1-yl)pyridin-3-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (400 mg, 90% purity, 0.915 mmol), N-benzyl-N,N-diethylethanaminium chloride (30 mg, 0.132 mmol) and mixture of 5-(1-bromoethyl)-2-(5-methyl-2H-tetrazol-2-yl)pyridine and 5-(1-bromoethyl)-2-(5-methyl-1H-tetrazol-1-yl)pyridine 26-3 (400 mg, 90% purity, 1.34 mmol) in 2-methyltetrahydrofuran (4 mL) was added 50% sodium hydroxide in water (4 mL) slowly at 30° C. After stirred at 30° C. for 4 hours, the reaction mixture was diluted with water (50 mL) and extracted with ethyl acetate (50 mL) twice. The combined organic layers were washed with brine (50 mL) twice, dried over Na₂SO₄₍ₛ₎ and filtered. The filtrate was concentrated and purified by silica gel column chromatography (petroleum ether:ethyl acetate=2:1) to give the title mixture (500 mg, 90% purity from ¹H NMR, 85% yield) as white solids. LC-MS (ESI): R_{T}=1.63 min, mass calcd. for C₂₇H₂₇Cl₂N₉O₂ 579.2, m/z found 580.1 [M+H]⁺.

Intermediates 26-5 and 26-6

### Mixture of (3R,7R)-2-(3,4-dichlorobenzoyl)-3,7-dimethyl-9-((R*)-1-(6-(5-methyl-211-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-((R*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one (26-5) and Mixture of (3R,7R)-2-(3,4-dichlorobenzoyl)-3,7-dimethyl-9-((S*)-1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-((S*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one (26-6)

The racemic mixture of (3R,7R)-2-(3,4-dichlorobenzoyl)-3,7-dimethyl-9-(1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-(1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one 26-4 (500 mg, 90% purity, 0.775 mmol) was separated by Chiral Prep-HPLC (Column: Chiralpak IA 10 μm 30 mm*250 mm; Mobile Phase: ACN:IPA=70:30 at 25 mL/min; Col. Temp: 30° C.; Wavelength: 254 nm) to afford the title mixture 26-5 (230 mg, 98.0% purity from LCMS, 50.1% yield) as white solids and the title mixture 26-6 (200 mg, 100% purity from LCMS, 44.4% yield) as white solids.

26-5:

LC-MS (ESI): R_{T}=1.62 min, mass calcd. for C₂₇H₂₇Cl₂N₉O₂ 579.2, m/z found 580.1 [M+H]⁺.

26-6:

LC-MS (ESI): R_{T}=1.63 min, mass calcd. for C₂₇H₂₇Cl₂N₉O₂ 579.2 m/z found 580.1 [M+H]⁺.

### Compounds 26A and 27A

### (3R,7R)-2-(3,4-dichlorobenzoyl)-3,7-dimethyl-9-((R*)-1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one (26A) and (3R,7R)-2-(3,4-dichlorobenzoyl)-3,7-dimethyl-9-((R*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one (27A)

A mixture of (3R,7R)-2-(3,4-dichlorobenzoyl)-3,7-dimethyl-9-((R*)-1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-((R*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one 26-5 (230 mg, 90% purity, 0.357 mmol) was separated by Chiral Prep-HPLC (Column: Chiralpak IC 10 μm 30 mm*250 mm; Mobile Phase: ACN:IPA=90:10 at 25 mL/min; Col. Temp: 30° C.; Wavelength: 254 nm) to afford the title compounds 27A (70 mg, 96.5% purity from LCMS, 32.6% yield, 100% stereopure) as white solids and 26A (45 mg, 99.7% purity from LCMS, 21.7% yield, 100% stereopure) as white solids.

### 27A:

LC-MS (ESI): R_{T}=3.766 min, mass calcd. for C₂₇H₂₇Cl₂N₉O₂, 579.2, m/z found 580.2. [M+H]⁺. Chiral analysis (Column: Chiralpak IE 5 μm 4.6*250 mm; Mobile Phase: ACN:IPA=70:30 at 1.0 mL/min; Temp: 30° C.; Wavelength: 254 nm; R_{T}=8.198 min). ¹H NMR (400 MHz, DMSO-d₆) δ 8.67 (br s, 1H), 8.19 (br s, 1H), 7.98 (d, J=8.0 Hz, 1H), 7.75 (d, J=8.0 Hz, 2H), 7.46-7.43 (m, 1H), 5.94-5.83 (m, 1H), 5.47-5.23 (m, 1H), 4.49 (br s, 2H), 4.17-4.10 (m, 1H), 3.88 (br s, 1H), 3.28-3.21 (m, 1H), 2.96-2.92 (m, 1H), 2.79 (s, 3H), 2.64-2.55 (m, 1H), 1.66 (br s, 3H), 1.31 (d, J=6.4 Hz, 3H), 1.12 (br s, 3H).

### 26A:

LC-MS (ESI): R_{T}=3.790 min, mass calcd. for C₂₇H₂₇Cl₂N₉O₂ 579.2, m/z found 552.2 [M−H₂O+H]⁺. Chiral analysis (Column: Chiralpak IA 5 μm, 4.6*250 mm; Mobile Phase: ACN:IPA=70:30 at 1 mL/min; Temp 30° C.; Wavelength: 254 nm; R_{T}=5.113 min). ¹H NMR (400 MHz, CDCl₃) δ 8.75-8.62 (m, 1H), 8.23-8.07 (m, 2H), 7.50 (d, J=8.0 Hz, 2H), 7.45 (dd, J=8.0, 2.0 Hz, 1H), 6.03-5.79 (m, 1H), 5.50-5.16 (m, 1H), 4.50-3.86 (m, 4H), 3.29-3.15 (m, 1H), 2.99-2.86 (m, 1H), 2.68-2.55 (m, 4H), 1.74-1.67 (m, 3H), 1.29 (d, J=6.4 Hz, 3H), 1.23-1.04 (m, 3H).

### Compounds 26B and 27B

### (3R,7R)-2-(3,4-dichlorobenzoyl)-3,7-dimethyl-9-((S*)-1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one (26B) and (3R,7R)-2-(3,4-dichlorobenzoyl)-3,7-dimethyl-9-((S*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one (27B)

A mixture of (3R,7R)-2-(3,4-dichlorobenzoyl)-3,7-dimethyl-9-((S*)-1-(6-(5-methyl-2H-tetrazol-2-yl)pyridin-3-yl)ethyl)-1,2,3,4,8,9-hexahydropyrido[4′,3′:3,4]pyrazolo[1,5-a]pyrazin-10(7H)-one and (3R,7R)-2-(3,4-dichlorobenzoyl)-3,7-dimethyl-9-((S*)-1-(6-(5-methyl-1H-tetrazol-1-yl)pyridin-3-yl)ethyl)-1,2,3,4,8,9-hexahydropyrido[4′,3′:3,4]pyrazolo[1,5-a]pyrazin-10(7H)-one 26-6 (200 mg, 90% purity, 0.310 mmol) was separated by Chiral Prep-HPLC (Column: Chiralpak IC 10 μm 30 mm*250 mm; Mobile Phase: ACN:IPA=70:30 at 25 mL/min; Col. Temp: 30° C.; Wavelength: 254 nm) to afford the title compound 27B (60 mg, 98.3% purity from Chiral HPLC, 32.8% yield, 98.3% stereopure) as white solids and 26B (35 mg, 97.9% purity from LCMS, 19.0% yield, 100% stereopure) as white solids.

### 27B:

LC-MS (ESI): R_{T}=3.790 min, Area %: 99.5 mass calcd. for C₂₇H₂₇Cl₂N₉O₂, 579.2, m/z found 580.2. [M+H]⁺. Chiral analysis (Column: Chiralpak IE 5 μm 4.6*250 mm; Mobile Phase: ACN:IPA=70:30 at 1.0 mL/min; Temp: 30° C.; Wavelength: 254 nm; R_{T}=9.494 min). ¹H NMR (400 MHz, DMSO-d₆) δ 8.65 (br s, 1H), 8.15 (br s, 1H), 7.99 (br s, 1H), 7.74 (d, J=8.4 Hz, 2H), 7.45-7.43 (m, 1H), 5.97-5.86 (m, 1H), 5.51-5.23 (m, 1H), 4.55-4.45 (m, 2H), 4.18-4.11 (m, 1H), 3.57 (br s, 2H), 2.96-2.91 (m, 1H), 2.79 (s, 3H), 2.64-2.54 (m, 1H), 1.66 (br s, 3H), 1.46 (d, J=6.4 Hz, 3H), 1.13 (br s, 3H).

### 26B:

LC-MS (ESI): R_{T}=3.813 min, mass calcd. for C₂₇H₂₇Cl₂N₉O₂ 579.2, m/z found 552.2 [M−H₂O+H]⁺. Chiral analysis (Column: Chiralpak IA 5 μm, 4.6*250 mm; Mobile Phase: ACN:IPA=70:30 at 1 mL/min; Temp 30° C.; Wavelength: 254 nm; R_{T}=10.005 min). ¹H NMR (400 MHz, CDCl₃) δ 8.73-8.61 (m, 1H), 8.23-7.99 (m, 2H), 7.74 (d, J=8.0 Hz, 2H), 7.45 (dd, J=8.0, 1.6 Hz, 1H), 5.98-5.82 (m, 1H), 5.55-5.17 (m, 1H), 4.59-4.16 (m, 3H), 3.64-3.45 (m, 2H), 2.96-2.91 (m, 1H), 2.70-2.54 (m, 4H), 1.74-1.57 (m, 3H), 1.46 (d, J=6.0 Hz, 3H), 1.22-1.04 (m, 3H).

### Compounds 28A and 28B

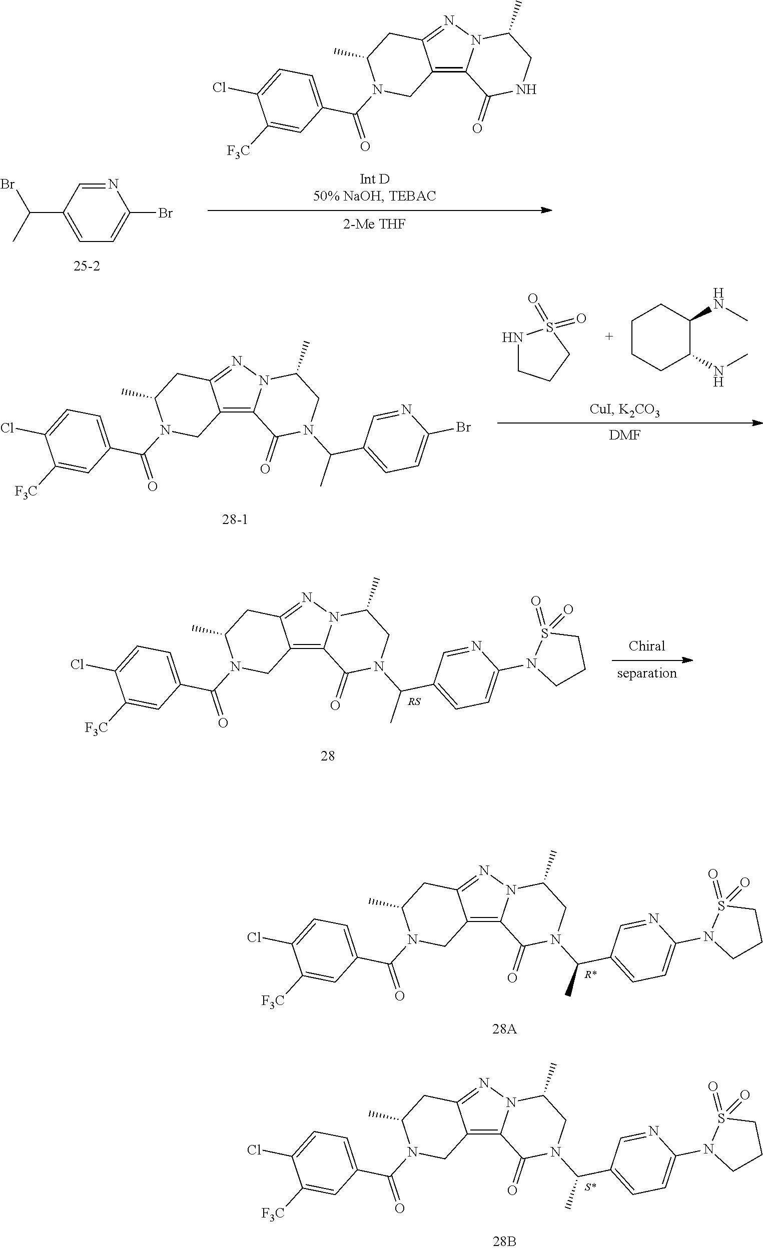

### Intermediate 28-1

### (3R,7R)-9-(1-(6-Bromopyridin-3-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one Int D (500 mg, 90% purity, 1.05 mmol) and 2-bromo-5-(1-bromoethyl)pyridine 25-2 (550 mg, 85% purity, 1.77 mmol) in 2-methyltetrahydrofuran (5 mL) were added 50% sodium hydroxide in water (5 mL) and benzyltriethylammonium chloride (70 mg, 0.307 mmol) slowly at room temperature. After stirred at room temperature for 5 hours, the mixture was added water (50 mL) and extracted with ethyl acetate (50 mL) for three times. The combined organic layers were washed with brine (50 mL) and concentrated to give a residue. The residue was purified by silica gel column chromatography (petroleum ether:ethyl acetate=1:1 to 0:1) to give the title compound (560 mg, 90% purity from ¹H NMR, 78% yield) as white solids.

### Compound 28

### (3R,7R)-2-(4-Chloro-3-(trifluoromethyl)benzoyl)-9-(1-(6-(1,1-dioxidoisothiazolidin-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-9-(1-(6-bromopyridin-3-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one 28-1 (170 mg, 90% purity, 0.25 mmol), isothiazolidine 1,1-dioxide (35 mg, 0.29 mmol) and potassium carbonate (80 mg, 0.58 mmol) in N,N-dimethylformamide (2 mL) were added copper(I) iodide (15 mg, 0.08 mmol) and (1R,2R)—N1,N2-dimethylcyclohexane-1,2-diamine (25 mg, 0.18 mmol) under the protection of nitrogen. Then the reaction mixture was stirred at 100° C. for 13 hours, then cooled to room temperature. The reaction mixture was quenched with brine (10 mL), extracted with acetate (10 mL) for three times and the combined organic layers were dried over Na₂SO₄₍ₛ₎, filtered. The filtrate was concentrated to get a residue, which was purified by C18 chromatography (acetonitrile:water (+0.02% ammonium bicarbonate)=40-60%) to give the title compound (170 mg, 74% purity from LCMS, 77.1% yield) as yellow solids. LC-MS (ESI): R_{T}=1.63 min, mass calcd. for C₂₉H₃₀ClF₃N₆O₅S 650.2 m/z found 651.0 [M+H]⁺.

### Compounds 28A and 28B

### (3R,7R)-2-(4-chloro-3-(trifluoromethyl)benzoyl)-9-((R*)-1-(6-(1,1-dioxidoisothiazolidin-2-yl)pyridin-3-yl)ethyl)-3,7-dimethyl-1,2,3,4,8,9-hexahydropyrido[4′,3′: 3,4]pyrazolo[1,5-a]pyrazin-10(7H)-one (28A) and (3R,7R)-2-(4-chloro-3-(trifluoromethyl)benzoyl)-9-((S*)-1-(6-(1,1-dioxidoisothiazolidin-2-yl)pyridin-3-yl)ethyl)-3,7-dimethyl-1,2,3,4,8,9-hexahydropyrido[4′,3′: 3,4]pyrazolo[1,5-a]pyrazin-10(7H)-one (28B)

The racemate of (3R,7R)-2-(4-chloro-3-(trifluoromethyl)benzoyl)-9-(1-(6-(1,1-dioxidoisothiazolidin-2-yl)pyridin-3-yl)ethyl)-3,7-dimethyl-1,2,3,4,8,9-hexahydro pyrido[4′,3′:3,4]pyrazolo[1,5-a]pyrazin-10(7H)-one 28 (170 mg, 74% purity, 0.19 mmol) was separated by chiral prep HPLC (separation condition: Column: Chiralpak ID 5 μm 20*250 mm; Mobile Phase: ACN:IPA=90:10 at 25 mL/min; Temp: 30° C.; Wavelength: 230 nm) to give the title compounds 28A (51.3 mg, 98.6% purity, 40.2% yield, 100% stereopure) and compound 28B (52.6 mg, 99.8% purity, 41.7% yield, 99.8% stereopure) as white solids.

### 28A:

LC-MS (ESI): R_{T}=3.403 min, mass calcd. for C₂₉H₃₀ClF₃N₆O₅S 650.2 m/z found 651.1 [M+H]⁺. Chiral analysis (Column: Chiralpak ID 5 μm 4.6*250 mm; Mobile Phase: ACN:IPA=90:10 at 1 mL/min; Temp: 30° C.; Wavelength: 230 nm, R_{T}=4.930 min). ¹H NMR (400 MHz, DMSO-d₆) δ 8.42-8.27 (m, 1H), 7.91 (s, 1H), 7.86-7.73 (m, 3H), 7.17 (d, J=8.4 Hz, 1H), 5.93-5.66 (m, 1H), 5.56-5.13 (m, 1H), 4.61-4.36 (m, 2H), 4.24-4.08 (m, 1H), 3.89 (t, J=6.4 Hz, 2H), 3.84-3.67 (m, 1H), 3.57 (t, J=7.2 Hz, 2H), 3.14-3.02 (m, 1H), 2.94 (dd, J=15.6, 6.0 Hz, 1H), 2.66-2.51 (m, 1H), 2.42-2.35 (m, 2H), 1.67-1.46 (m, 3H), 1.25 (d, J=6.4 Hz, 3H), 1.22-1.06 (m, 3H). ¹⁹F NMR (376 MHz, DMSO-d₆) δ −61.33.

### 28B:

LC-MS (ESI): R_{T}=3.661 min, mass calcd. for C₂₉H₃₀ClF₃N₆O₅S 650.2 m/z found 651.2 [M+H]⁺. Chiral analysis (Column: Chiralpak ID 5 μm 4.6*250 mm; Mobile Phase: ACN:IPA=90:10 at 1 mL/min; Temp: 30° C.; Wavelength: 230 nm, R_{T}=6.094 min). ¹H NMR (400 MHz, DMSO-d₆) δ 8.40-8.25 (m, 1H), 7.91 (s, 1H), 7.86-7.70 (m, 3H), 7.21-7.01 (m, 1H), 5.92-5.62 (m, 1H), 5.57-5.14 (m, 1H), 4.64-4.28 (m, 2H), 4.24-4.07 (m, 1H), 3.89 (t, J=6.0 Hz, 2H), 3.56 (t, J=7.6 Hz, 2H), 3.52-3.35 (m, 2H), 2.94 (dd, J=16.4, 5.6 Hz, 1H), 2.66-2.51 (m, 1H), 2.42-2.34 (m, 2H), 1.64-1.49 (m, 3H), 1.43 (d, J=6.4 Hz, 3H), 1.27-1.06 (m, 3H). ¹⁹F NMR (376 MHz, DMSO-d₆) δ −61.33.

### Compounds 29A, 29B, 29C and 29D

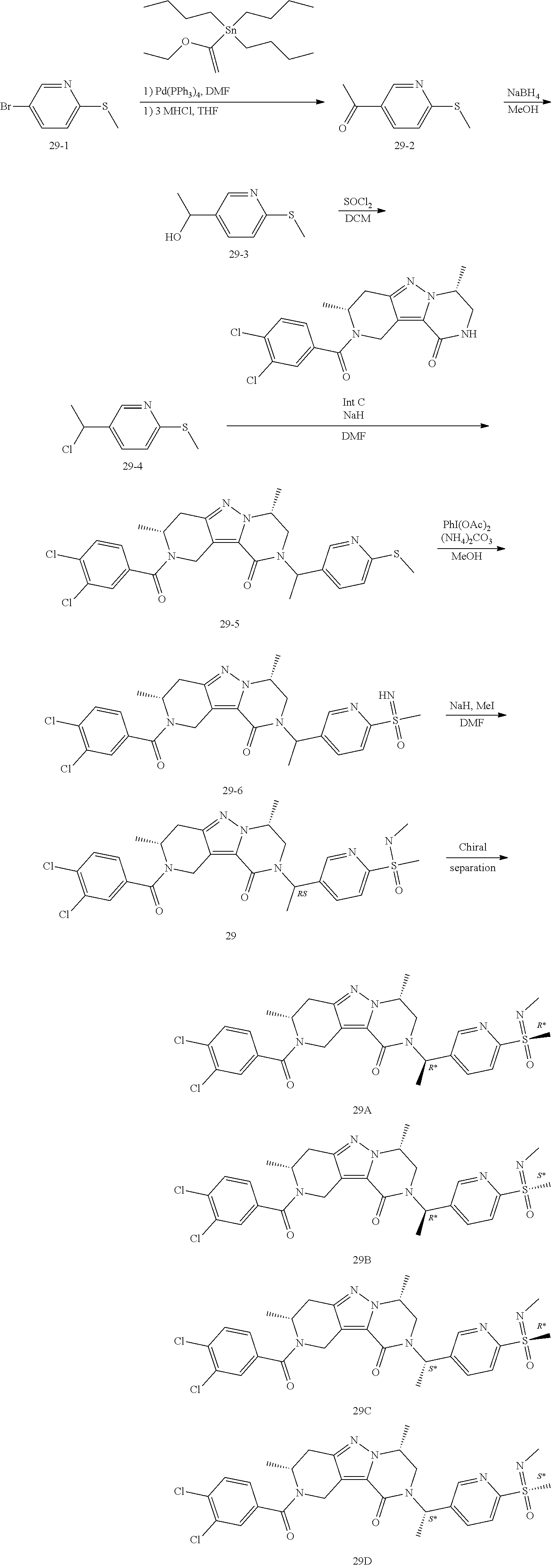

### Intermediate 29-2

### 1-(6-(Methylthio)pyridin-3-yl)ethanone

To a solution of 5-bromo-2-(methylthio)pyridine 29-1 (2 g, 9.80 mmol) and tributyl(1-ethoxyvinyl)stannane (4.3 g, 11.9 mmol) in N,N-dimethylformamide (30 mL) was added tetrakis(triphenylphosphine)palladium (566 mg, 0.490 mmol) under nitrogen atmosphere. The reaction mixture was cooled to room temperature after stirred at 100° C. for 2 hours. Then the mixture was diluted with saturated potassium fluoride aqueous solution (100 mL) and filtered. The filtrate was extracted with ethyl acetate (200 mL) for three times. The combined organic layers were washed with brine (80 mL), dried over Na₂SO₄₍ₛ₎ and filtered. The filtrate was concentrated to give a residue, which was diluted with tetrahydrofuran (20 mL), 3 M hydrochloride aqueous solution (6.2 mL, 18.6 mmol) was added into the mixture. After stirred at 0° C. for 2 hours, the reaction mixture was extracted with ethyl acetate (30 mL) twice. The combined organic layers were washed with brine (50 mL), dried over Na₂SO₄₍ₛ₎ and filtered. The filtrate was concentrated in vacuum to give a residue. Which was purified by silica gel column chromatography (petroleum ether:ethyl acetate=5:1) to give the title compound (1.2 g, 90% purity from ¹H NMR, 69% yield) as yellow oil. ¹H NMR (400 MHz, CDCl₃) δ 8.98-8.97 (m, 1H), 8.01 (dd, J=8.4, 2.0 Hz, 1H), 7.27-7.26 (m, 1H) 2.61 (s, 3H), 2.59 (s, 3H).

### Intermediate 29-3

### 1-(6-(Methylthio)pyridin-3-yl)ethanol

To a solution of 1-(6-(methylthio)pyridin-3-yl)ethanone 29-2 (500 mg, 90% purity, 2.69 mmol) in methanol (5 mL) was added sodium borohydride (40 mg, 1.06 mmol) at 0° C. After stirred at 0° C. for 0.5 hour, the reaction mixture was quenched with saturated ammonium chloride aqueous solution (2 mL), dried over Na₂SO₄₍ₛ₎ and filtered. The filtrate was concentrated in vacuum to give a residue, which was purified by silica gel column chromatography (petroleum ether:ethyl acetate=1:1) to give the title compound (450 mg, 90% purity from ¹H NMR, 89% yield) as yellow oil. ¹H NMR (400 MHz, CDCl₃) δ 8.38 (s, 1H), 7.35 (dd, J=8.4 Hz, 2.4 Hz, 1H), 7.15 (d, J=8.4 Hz, 1H) 4.88 (q, J=6.4 Hz, 1H), 2.55 (s, 3H), 1.49 (d, J=6.8 Hz, 3H).

### Intermediate 29-4

### 5-(1-Chloroethyl)-2-(methylthio)pyridine

To a solution of 1-(6-(methylthio)pyridin-3-yl)ethanol 29-3 (450 mg, 90% purity, 2.39 mmol) in dichloromethane (6 mL) was added sulfurous dichloride (854 mg, 7.18 mmol). After stirring at 40° C. for 0.5 hour, the mixture was concentrated under reduced pressure to get the title compound (480 mg, 85% purity from ¹H NMR, 91% yield) as colorless oil. ¹H NMR (400 MHz, CDCl₃) δ 8.68 (s, 1H), 8.05 (d, J=7.6 Hz, 1H), 7.35 (d, J=8.4 Hz, 1H), 5.13 (q, J=6.8 Hz, 1H), 2.92 (s, 3H), 1.88 (d, J=6.8 Hz, 3H).

### Intermediate 29-5

### (3R,7R)-2-(3,4-Dichlorobenzoyl)-3,7-dimethyl-9-(1-(6-(methylthio)pyridin-3-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (790 mg, 90% purity, 1.81 mmol) in N,N-dimethylformamide (10 mL) was added 60% wt. sodium hydride in mineral oil (289 mg, 7.23 mmol). After stirred at 0° C. for 0.5 hour, 5-(1-chloroethyl)-2-(methylthio)pyridine 29-4 (480 mg, 85% purity, 2.17 mmol) in N,N-dimethylformamide (2 mL) was added into the mixture. After stirred at 0° C. for 1 hour, the mixture was diluted with water (50 mL) and extracted with ethyl acetate (50 mL) twice. The combined organic layers were washed with brine (50 mL), dried over Na₂SO₄₍ₛ₎ and filtered. The filtrate was concentrated to give a residue, which was purified by C18 column (acetonitrile:water=45% to 55%) to give the title compound (550 mg, 95% purity from LCMS, 53% yield) as white solids. LC-MS (ESI): R_{T}=1.72 min, mass calcd. for C₂₆H₂₇Cl₂N₅O₂S 543.1 mz found 544.0 [M+H]⁺.

### Intermediate 29-6

### (3R,7R)-2-(3,4-dichlorobenzoyl)-3,7-dimethyl-9-(1-(6-(S-methylsulfonimidoyl)pyridin-3-yl)ethyl)-1,2,3,4,8,9-hexahydropyrido[4′,3′:3,4]pyrazolo[1,5-a]pyrazin-10(7H)-one

To a mixture of (3R,7R)-2-(3,4-dichlorobenzoyl)-3,7-dimethyl-9-(1-(6-(methylthio)pyridin-3-yl)ethyl)-1,2,3,4,8,9-hexahydropyrido[4′,3′:3,4]pyrazolo[1,5-a]pyrazin-10(7H)-one 29-5 (550 mg, 95% purity, 0.960 mmol) and ammonium carbonate (138 mg, 1.44 mmol) in methanol (10 mL) was added [bis(acetoxy)iodo]benzene (680 mg, 2.11 mmol). After stirred at 20° C. for 10 minutes, the reaction mixture was concentrated in vacuum to get a residue, which was diluted with water (20 mL) and extracted with ethyl acetate (40 mL) twice. The combined organic layers were washed with brine (20 mL), dried over Na₂SO₄₍ₛ₎ and filtered. The filtrate was concentrated in vacuum to give a residue, which was purified by column chromatography (petroleum ether:ethyl acetate=1:2) to give the title compound (450 mg, 100% purity from LCMS, 81% yield) as yellow solids. LC-MS (ESI): R_{T}=1.48 min, mass calcd. for C₂₆H₂₈Cl₂N₆O₃S 574.1 mz found 575.1 [M+H]⁺.

### Compound 29

### (3R,7R)-2-(3,4-dichlorobenzoyl)-9-(1-(6-(N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-9-(1-(6-(S-methylsulfonimidoyl)pyridin-3-yl)ethyl)-1,2,3,4,8,9-hexahydropyrido[4′,3′:3,4]pyrazolo[1,5-a]pyrazin-10(7H)-one 29-6 (450 mg, 100% purity, 0.782 mmol) in N,N-dimethylformamide (8 mL) was added 60% wt. sodium hydride in mineral oil (63 mg, 1.58 mmol). After stirred at 0° C. for 0.5 hour, iodomethane (185 mg, 1.17 mmol) in N,N-dimethylformamide (2 mL) was added into the mixture. After stirred at 0° C. for 1 hour, the mixture was diluted with water (50 mL) and extracted with ethyl acetate (50 mL) twice. The combined organic layers were washed with brine (50 mL), dried over Na₂SO₄₍ₛ₎ and filtered. The filtrate was concentrated to give a residue, which was purified by C18 column (acetonitrile:water=45% to 55%) to give the title compound (380 mg, 100% purity from LCMS, 82% yield) as white solids. LC-MS (ESI): R_{T}=1.50 min, mass calcd. for C₂₇H₃₀Cl₂N₆O₃S 588.2 mz found 589.1 [M+H]⁺.

### Compounds 29A, 29B, 29C and 29D

### (3R,7R)-2-(3,4-dichlorobenzoyl)-9-((R*)-1-(6-((R*)—N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one (29A), (3R,7R)-2-(3,4-dichlorobenzoyl)-9-((R*)-1-(6-((S*)—N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one (29B), (3R,7R)-2-(3,4-dichlorobenzoyl)-9-((S*)-1-(6-((R*)—N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one (29C) and (3R,7R)-2-(3,4-dichlorobenzoyl)-9-((S*)-1-(6-((S*)—N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one (29D)

A racemic mixture of (3R,7R)-2-(3,4-dichlorobenzoyl)-9-(1-(6-(N,S-dimethylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one 29 (380 mg, 100% purity, 0.645 mmol) was separated by chiral HPLC (separation condition: Column Chiralpak IE 5 m 20*250 mm; Mobile Phase: ACN:IPA:DEA=60:40:0.2 at 15 mL/min; Temp: 30° C.; Wavelength: 254 nm) to give Fraction I (160 mg, 100% purity, 42% yield, 100% stereopure) as colorless oil and Fraction II (170 mg, 100% purity, 45% yield, 99.9% stereopure) as colorless oil. Fraction I (160 mg, 100% purity, 42% yield, 100% stereopure) was separated by chiral HPLC (separation condition: Column Chiralpak IC 5 μm 20*250 mm; Mobile Phase: MeOH:DCM=60:40 at 15 mL/min; Temp: 30° C.; Wavelength: 254 nm) to give the title compounds 29A (49.0 mg, 98.8% purity, 30% yield, 100% stereopure) as white solids and 29B (31.0 mg, 98.9% purity, 19% yield, 99.9% stereopure) as white solids. Fraction I (170 mg, 100% purity, 45% yield, 99.9% stereopure) as colorless oil was separated by chiral HPLC (separation condition: Column Chiralpak IB 5 μm 20*250 mm; Mobile Phase: ACN:IPA:DEA=90:10:0.2 at 15 mL/min; Temp: 30° C.; Wavelength: 254 nm) to give the title compounds 29C (50.5 mg, 99.0% purity, 29% yield, 100% stereopure) as white solids and 29D (40.3 mg, 99.8% purity, 24% yield, 100% stereopure) as white solids.

### 29A:

LC-MS (ESI): R_{T}=3.415 min, mass calcd. for C₂₇H₃₀Cl₂N₆O₃S 588.2 mz found 589.2 [M+H]⁺. Chiral analysis (Column: Superchiral IC 5 m 4.6*250 mm; Mobile Phase: MeOH:DCM=60:40 at 1 mL/min; Temp: 30° C.; Wavelength: 254 nm; Rt=5.919 min). ¹H NMR (400 MHz, CDCl₃) δ 8.77 (s, 1H), 8.11 (d, J=8.0 Hz, 1H), 7.94-7.93 (m, 1H), 7.54-7.50 (m, 2H), 7.28-7.25 (m, 1H), 6.15 (br s, 1H), 5.73-5.34 (m, 1H), 4.93-4.27 (m, 3H), 3.71-3.68 (m, 1H), 3.25 (s, 3H), 3.05-3.01 (m, 2H), 2.72-2.66 (m, 4H), 1.68 (d, J=6.4 Hz, 3H), 1.34 (d, J=6.4 Hz, 3H), 1.27 (d, J=4.8 Hz, 3H).

### 29B:

LC-MS (ESI): R_{T}=3.432 min, mass calcd. for C₂₇H₃₀Cl₂N₆O₃S 588.2 mz found 589.2 [M+H]⁺. Chiral analysis (Column: Superchiral IC 5 m 4.6*250 mm; Mobile Phase: MeOH:DCM=60:40 at 1 mL/min; Temp: 30° C.; Wavelength: 254 nm; Rt=7.551 min). ¹H NMR (400 MHz, CDCl₃) δ 8.77 (s, 1H), 8.10 (d, J=8.0 Hz, 1H), 7.95-7.93 (m, 1H), 7.54-7.50 (m, 2H), 7.28-7.25 (m, 1H), 6.16 (br s, 1H), 5.75-5.39 (m, 1H), 4.88-4.27 (m, 3H), 3.75-3.64 (m, 1H), 3.24 (s, 3H), 3.07-3.02 (m, 2H), 2.71-2.67 (m, 4H), 1.69 (d, J=6.8 Hz, 3H), 1.34 (d, J=6.4 Hz, 3H), 1.27-1.26 (m, 3H).

### 29C:

LC-MS (ESI): R_{T}=2.779 min, mass calcd. for C₂₇H₃₀Cl₂N₆O₃S 588.2 mz found 589.2 [M+H]⁺. Chiral analysis (Column: Superchiral IB 5 m 4.6*250 mm; Mobile Phase: ACN:IPA:DEA=90:10:0.2 at 1 mL/min; Temp: 30° C.; Wavelength: 254 nm; Rt=6.586 min). ¹H NMR (400 MHz, CDCl₃) δ 8.73 (s, 1H), 8.09 (d, J=8.0 Hz, 1H), 7.94-7.86 (m, 1H), 7.54-7.51 (m, 2H), 7.28-7.26 (m, 1H), 6.16 (br s, 1H), 5.86-5.39 (m, 1H), 5.03-4.36 (m, 3H), 3.39-3.28 (m, 2H), 3.24 (s, 3H), 3.07-2.93 (m, 1H), 2.73-2.66 (m, 4H), 1.69 (d, J=6.8 Hz, 3H), 1.58 (d, J=6.8 Hz, 3H), 1.27 (d, J=6.0 Hz, 3H).

### 29D:

LC-MS (ESI): R_{T}=2.785 min, mass calcd. for C₂₇H₃₀Cl₂N₆O₃S 588.2 mz found 589.2 [M+H]⁺. Chiral analysis (Column: Superchiral IB 5 m 4.6*250 mm; Mobile Phase: ACN:IPA:DEA=90:10:0.2 at 1 mL/min; Temp: 30° C.; Wavelength: 254 nm; Rt=8.225 min). ¹H NMR (400 MHz, CDCl₃) δ 8.73 (s, 1H), 8.09 (d, J=8.0 Hz, 1H), 7.94-7.86 (m, 1H), 7.54-7.51 (m, 2H), 7.28-7.26 (m, 1H), 6.16 (br s, 1H), 5.86-5.39 (m, 1H), 5.03-4.36 (m, 3H), 3.39-3.28 (m, 2H), 3.24 (s, 3H), 3.07-2.93 (m, 1H), 2.73-2.66 (m, 4H), 1.69 (d, J=6.8 Hz, 3H), 1.58 (d, J=6.8 Hz, 3H), 1.27 (d, J=6.0 Hz, 3H).

### Compounds 30A, 30B, 30C and 30D

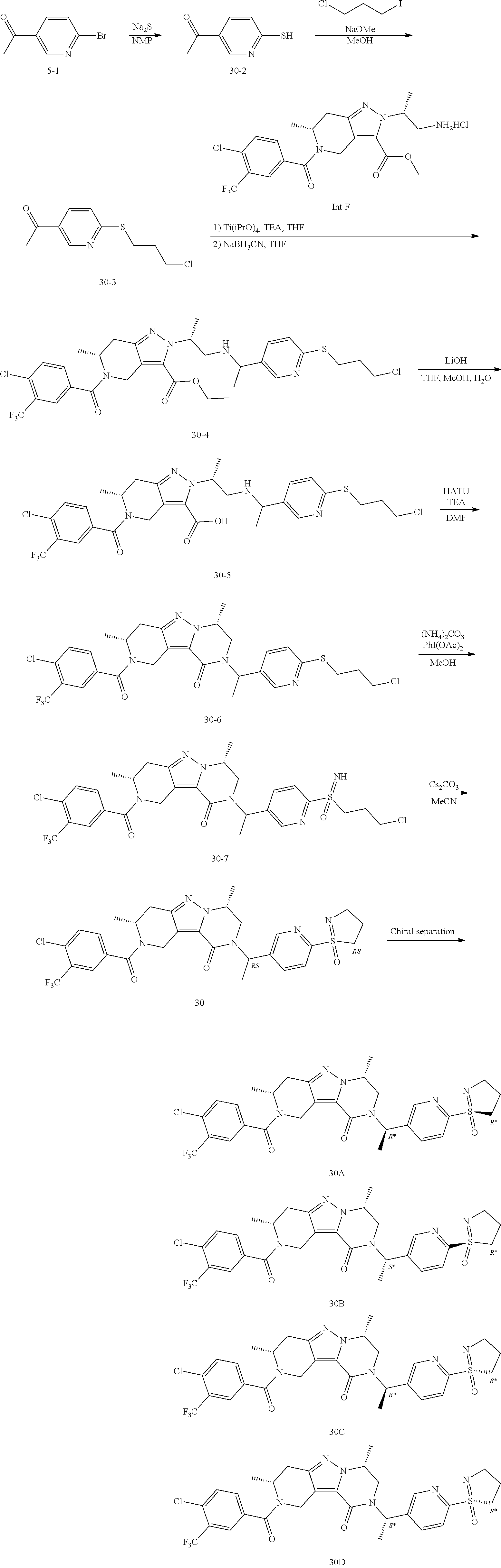

### Intermediate 30-2

### 1-(6-Mercaptopyridin-3-yl)ethan-1-one

Sodium sulfide (5.0 g, 64.07 mmol) was dissolved in 1-methylpyrrolidin-2-one (20 mL) and heated to 140° C. The mixture was partially evaporated under vacuum to remove the water. After cooled to room temperature, a solution of 1-(6-bromopyridin-3-yl)ethan-1-one 5-1 (5.0 g, 24.9 mmol) in 1-methylpyrrolidin-2-one (10 mL) was added and the reaction mixture was stirred at 70° C. under nitrogen atmosphere for 16 hours. Then the mixture was diluted with water (100 mL) after cooled to room temperature and extracted with ethyl acetate (100 mL) twice. The combined organic layers were washed with brine (100 mL), dried over Na₂SO₄₍ₛ₎ and filtered. The filtrate was concentrated in vacuum to give the title compound (2.5 g, 92% purity from LCMS, 60% yield) as a brown oil. LC-MS (ESI): Rt=0.91 min, mass calcd. for C₇H₇NOS, 153.2, m/z found 154.0 [M+H]⁺.

### Intermediate 30-3

### 1-(6-((3-Chloropropyl)thio)pyridin-3-yl)ethan-1-one

To a stirred solution of 1-(6-mercaptopyridin-3-yl)ethan-1-one 30-2 (2.0 g, 92% purity, 12.0 mmol) in anhydrous methanol (25 mL) were added 1-chloro-3-iodopropane (4.0 g, 19.6 mmol) and sodium methoxide (800 mg, 14.8 mmol) at room temperature. The reaction mixture was stirred at 60° C. for 2 hours, then was quenched with water (50 mL) and extracted with ethyl acetate (50 mL) twice. The combined organic layers were washed with brine (50 mL), dried over Na₂SO₄₍ₛ₎, filtered. The filtrate was concentrated in vacuum to give the title compound (650 mg, 68% purity from LCMS, 18.1% yield) as brown oil. LC-MS (ESI): Rt=1.60 min, mass calcd. for C₁₀H₁₂ClNOS, 229.7, m/z found 230.1 [M+H]⁺.

### Intermediate 30-4

### Ethyl (6R)-5-(4-chloro-3-(trifluoromethyl)benzoyl)-2-((2R)-1-((1-(6-((3-chloropropyl)thio)pyridin-3-yl)ethyl)amino)propan-2-yl)-6-methyl-4,5,6,7-tetrahydro-2H-pyrazolo[4,3-c]pyridine-3-carboxylate

To a solution of ethyl R-2-((R)-1-aminopropan-2-yl)-5-(4-chloro-3-(trifluoromethyl)benzoyl)-6-methyl-4,5,6,7-tetrahydro-2H-pyrazolo[4,3-c]pyridine-3-carboxylate hydrochloride Int F (700 mg, 100% purity, 1.48 mmol) and 1-(6-((3-chloropropyl)thio)pyridine-3-yl)ethan-1-one 30-3 (350 mg, 68% purity, 1.03 mmol) in tetrahydrofuran (20 mL) were added tetraisopropoxytitanium (1.2 g, 4.22 mmol) and triethylamine (50 mg, 0.494 mmol) at 30° C. The reaction mixture was heated to 70° C. for 3 hours, then added sodium cyanoborohydride (200 mg, 3.18 mmol) at 0° C. and stirred for 2 hours. The reaction mixture was quenched with water (20 mL) and extracted with ethyl acetate (20 mL) for three times. The combined organic layers were washed with brine (30 mL), dried over Na₂SO₄₍ₛ₎, filtered. The filtrate was concentrated in vacuum to give a residue, which was purified by C18 column (acetonitrile water=50% to 60%) to give the title compound (183 mg, 70% purity from LCMS, 26% yield) as colorless oil. LC-MS (ESI): Rt=1.74 min, mass calcd. for C₃₁H₃₆Cl₂F₃N₅03S, 685.2, m/z found 686.0 [M+H]⁺.

### Intermediate 30-5

### (6R)-5-(4-chloro-3-(trifluoromethyl)benzoyl)-2-((2R)-1-((1-(6-((3-chloropropyl)thio)pyridin-3-yl)ethyl)amino)propan-2-yl)-6-methyl-4,5,6,7-tetrahydro-2H-pyrazolo[4,3-c]pyridine-3-carboxylic acid

To a solution of ethyl (6R)-5-(4-chloro-3-(trifluoromethyl)benzoyl)-2-((2R)-1-((1-(6-((3-chloropropyl)thio)pyridin-3-yl)ethyl)amino)propan-2-yl)-6-methyl-4,5,6,7-tetrahydro-2H-pyrazolo[4,3-c]pyridine-3-carboxylate 30-4 (600 mg, 70% purity, 0.611 mmol) in tetrahydrofuran (2 mL) and methanol (1 mL) was added lithium hydroxide hydrate (60 mg, 1.43 mmol) in water (1 mL) at 0° C. The mixture was stirred at 0° C. for 2 hours. The mixture was diluted with water (10 mL), acidized with 0.5 M hydrochloride aqueous solution to pH˜5 and extracted with ethyl acetate (10 mL) for three times. The combined organic layers were washed with brine (10 mL), dried over Na₂SO₄₍ₛ₎ and filtered. The filtrate was concentrated to give the title compound (550 mg, 68% purity from LCMS, 82.3% yield) as yellow solids. LC-MS (ESI): R_{T}=1.52 min, mass calcd. for C₂₉H₃₂Cl₂F₃N₅03S 657.2 m/z found 657.9 [M+H]⁺.

### Intermediate 30-6

### (3R,7R)-2-(4-chloro-3-(trifluoromethyl)benzoyl)-9-(1-(6-((3-chloropropyl)thio) pyridin-3-yl)ethyl)-3,7-dimethyl-1,2,3,4,8,9-hexahydropyrido[4′,3′:3,4]pyrazolo [1,5-a]pyrazin-10(7H)-one

To a solution of (6R)-5-(4-chloro-3-(trifluoromethyl)benzoyl)-2-((2R)-1-((1-(6-((3-chloropropyl)thio)pyridin-3-yl)ethyl)amino)propan-2-yl)-6-methyl-4,5,6,7-tetrahydro-2H-pyrazolo[4,3-c]pyridine-3-carboxylic acid 30-5 (550 mg, 68% purity, 0.57 mmol) in N,N-dimethylformamide (5 mL) was added 2-(3H-[1,2,3]triazolo[4,5-b]pyridin-3-yl)-1,1,3,3-tetramethylisouronium hexafluorophosphate(V) (350 mg, 0.92 mmol). After stirred at 0° C. for 10 minutes, triethylamine (200 mg, 1.98 mmol) in N,N-dimethylformamide (2 mL) was added dropwise to the reaction at 0° C. Then the reaction mixture was stirred at 0° C. for 3 hours, quenched with brine (10 mL), extracted with ethyl acetate (10 mL) for three times. The combined organic layers were dried over Na₂SO₄₍ₛ₎, and filtered. The filtrate was concentrated to give a residue, which was purified by C18 chromatography (acetonitrile:water (+0.02% ammonium bicarbonate)=40-60%) to give the title compound (320 mg, 92% purity from LCMS, 80.9% yield) as yellow solids. LC-MS (ESI): R_{T}=1.79 min, mass calcd. for C₂₉H₃₀Cl₂F₃N₅O₂S 639.1 m/z found 640.0 [M+H]⁺.

### Intermediate 30-7

### (3R,7R)-2-(4-chloro-3-(trifluoromethyl)benzoyl)-9-(1-(6-(3-chloropropylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-9-(1-(6-((3-chloropropyl)thio)pyridin-3-yl)ethyl)-3,7-dimethyl-1,2,3,4,8,9-hexahydropyrido [4′,3′:3,4]pyrazolo[1,5-a]pyrazin-10(7H)-one 30-6 (300 mg, 92% purity, 0.43 mmol) in dry methanol (7 mL) was added iodobenzene diacetate (420 mg, 1.3 mmol) at 0° C. After stirring for 5 minutes, ammonium carbonate (130 mg, 1.35 mmol) was added at 0° C. The resulted reaction mixture was stirred at 20° C. for 14 hours. Then the reaction mixture was quenched with brine (10 mL), extracted with ethyl acetate (20 mL) for three times. The combined organic layers were dried over Na₂SO₄₍ₛ₎, filtered. The filtrate was concentrated to give a residue, which was purified by C18 chromatography (acetonitrile:water (+0.02% ammonium bicarbonate)=40-60%) to give the title compound (230 mg, 67% purity from LCMS, 53.3% yield) as yellow solids. LC-MS (ESI): R_{T}=1.55 min, mass calcd. for C₂₉H₃₁Cl₂F₃N₆03S 670.2 m/z found 671.0 [M+H]⁺.

### Compound 30

### (3R,7R)-2-(4-chloro-3-(trifluoromethyl)benzoyl)-3,7-dimethyl-9-(1-(6-(1-oxido-4,5-dihydro-3H-116-isothiazol-1-yl)pyridin-3-yl)ethyl)-1,2,3,4,8,9-hexahydro pyrido[4′,3′:3,4]pyrazolo[1,5-a]pyrazin-10(7H)-one

To a solution of (3R,7R)-2-(4-chloro-3-(trifluoromethyl)benzoyl)-9-(1-(6-(3-chloropropylsulfonimidoyl)pyridin-3-yl)ethyl)-3,7-dimethyl-1,2,3,4,8,9-hexahydropyrido[4′,3′:3,4]pyrazolo[1,5-a]pyrazin-10(7H)-one 30-7 (230 mg, 67% purity, 0.23 mmol) in acetonitrile (2 mL) was added cesium carbonate (290 mg, 0.89 mmol) at 25° C. The resulting mixture was stirred at 70° C. for 1 hour. After cooled to room temperature the reaction mixture was quenched with brine (10 mL), extracted with ethyl acetate (10 mL) for three times. The combined organic layers were dried over Na₂SO₄₍ₛ₎, filtered. The filtrate was concentrated to give a residue, which was purified by prep. TLC (dichloromethane:methanol=10:1) to give the title compound (150 mg, 84% purity from LCMS, 86.5% yield) as white solids. LC-MS (ESI): R_{T}=1.48 min, mass calcd. for C₂₉H₃₀ClF₃N₆O₃S 634.2 m/z found 634.9 [M+H]⁺.

### Compounds 30A, 30B, 30C and 30D

### (3R,7R)-2-(4-chloro-3-(trifluoromethyl)benzoyl)-3,7-dimethyl-9-((R*)-1-(6-((R*)-1-oxido-4,5-dihydro-3H-116-isothiazol-1-yl)pyridin-3-yl)ethyl)-1,2,3,4,8,9-hexahydropyrido[4′,3′:3,4]pyrazolo[1,5-a]pyrazin-10(7H)-one (30A), (3R,7R)-2-(4-chloro-3-(trifluoromethyl)benzoyl)-3,7-dimethyl-9-((S*)-1-(6-((R*)-1-oxido-4,5-dihydro-3H-116-isothiazol-1-yl)pyridin-3-yl)ethyl)-1,2,3,4,8,9-hexahydropyrido[4′,3′:3,4]pyrazolo[1,5-a]pyrazin-10(7H)-one (30B), (3R,7R)-2-(4-chloro-3-(trifluoromethyl)benzoyl)-3,7-dimethyl-9-((R*)-1-(6-((S*)-1-oxido-4,5-dihydro-3H-116-isothiazol-1-yl)pyridin-3-yl)ethyl)-1,2,3,4,8,9-hexahydropyrido[4′,3′:3,4]pyrazolo[1,5-a]pyrazin-10(7H)-one (30C) and (3R,7R)-2-(4-chloro-3-(trifluoromethyl)benzoyl)-3,7-dimethyl-9-((S*)-1-(6-((S*)-1-oxido-4,5-dihydro-3H-116-isothiazol-1-yl)pyridin-3-yl)ethyl)-1,2,3,4,8,9-hexahydropyrido[4′,3′:3,4]pyrazolo[1,5-a]pyrazin-10(7H)-one (30D)

The racemate of (3R,7R)-2-(4-chloro-3-(trifluoromethyl)benzoyl)-3,7-dimethyl-9-(1-(6-(1-oxido-4,5-dihydro-3H-116-isothiazol-1-yl)pyridin-3-yl)ethyl)-1,2,3,4,8,9-hexahydropyrido[4′,3′:3,4]pyrazolo[1,5-a]pyrazin-10(7H)-one 30 (160 mg, 84% purity, 0.21 mmol) was separated by chiral prep. HPLC (separation condition: Column: Chiralpak IC 5 μm 20*250 mm; Mobile Phase: ACN:IPA=50:50 at 25 mL/min; Temp: 30° C.; Wavelength: 230 nm) to give the title compounds 30-peak 1 (70 mg, 90% purity from ¹H NMR, 46.9% yield) and 30-peak 2 (70 mg, 90% purity from ¹H NMR, 46.9% yield) all as white solids.

The racemate (3R,7R)-2-(4-chloro-3-(trifluoromethyl)benzoyl)-3,7-dimethyl-9-(1-(6-((R*)-1-oxido-4,5-dihydro-3H-isothiazol-1-yl)pyridin-3-yl)ethyl)-1,2,3,4,8,9-hexahydropyrido[4′,3′:3,4]pyrazolo[1,5-a]pyrazin-10(7H)-one 30-peak 1 (70 mg, 90% purity, 0.1 mmol) was separated by chiral prep HPLC (separation condition: Column: Chiralpak IF 5 μm 20*250 mm; Mobile Phase: IF, ACN:IPA=50:50 at 25 mL/min; Temp: 30° C.; Wavelength: 254 nm) to give the title compounds 30A (18.1 mg, 96% purity, 27.6% yield, 100% stereopure) and 30B (14.6 mg, 96.1% purity, 22.3% yield, 99.9% stereopure) as white solids.

The racemate of (3R,7R)-2-(4-chloro-3-(trifluoromethyl)benzoyl)-3,7-dimethyl-9-(1-(6-((S*)-1-oxido-4,5-dihydro-3H-isothiazol-1-yl)pyridin-3-yl)ethyl)-1,2,3,4,8,9-hexahydropyrido[4′,3′:3,4]pyrazolo[1,5-a]pyrazin-10(7H)-one 30-peak 2 (70 mg, 90% purity, 0.1 mmol) was separated by chiral prep HPLC (separation condition: Column: Chiralpak IF 5 μm 20*250 mm; Mobile Phase: IF, ACN:IPA=50:50 at 25 mL/min; Temp: 30° C.; Wavelength: 254 nm) to give the title compounds 30C (18.1 mg, 97.6% purity, 28.0% yield, 100% stereopure) and 30D (14.5 mg, 97.7% purity, 22.5% yield, 99.9% stereopure) as white solids.

### 30A:

LC-MS (ESI): R_{T}=3.471 min, mass calcd. for C₂₉H₃₀ClF₃N₆O₃S 634.2 m/z found 635.3 [M+H]⁺. Chiral analysis (Column: Chiralpak IF 5 μm 4.6*250 mm; Mobile Phase: ACN:IPA=50:50 at 1 mL/min; Temp: 30° C.; Wavelength: 254 nm, R_{T}=5.417 min). ¹H NMR (400 MHz, DMSO-d₆) δ 8.83 (br s, 1H), 8.15-8.01 (m, 2H), 7.91 (s, 1H), 7.84 (d, J=8.0 Hz, 1H), 7.79 (d, J=8.4 Hz, 1H), 5.96-5.72 (m, 1H), 5.54-5.15 (m, 1H), 4.58-4.40 (m, 2H), 4.25-4.10 (m, 1H), 3.87-3.74 (m, 3H), 3.53-3.47 (m, 1H), 3.29-3.17 (m, 2H), 2.97-2.91 (m, 1H), 2.64-2.54 (m, 1H), 2.38-2.17 (m, 2H), 1.74-1.54 (m, 3H), 1.32 (d, J=6.4 Hz, 3H), 1.24-1.05 (m, 3H). ¹⁹F NMR (376 MHz, DMSO-d₆) δ −61.33.

### 30B:

LC-MS (ESI): R_{T}=3.542 min, mass calcd. for C₂₉H₃₀ClF₃N₆O₃S 634.2 m/z found 635.2 [M+H]⁺. Chiral analysis (Column: Chiralpak IF 5 μm 4.6*250 mm; Mobile Phase: ACN:IPA=50:50 at 1 mL/min; Temp: 30° C.; Wavelength: 254 nm, R_{T}=6.965 min). ¹H NMR (400 MHz, DMSO-d₆) δ 8.80 (br s, 1H), 8.12-7.97 (m, 2H), 7.91 (s, 1H), 7.84 (d, J=8.0 Hz, 1H), 7.79 (d, J=8.0 Hz, 1H), 6.00-5.74 (m, 1H), 5.59-5.13 (m, 1H), 4.60-4.38 (m, 2H), 4.24-4.10 (m, 1H), 3.87-3.73 (m, 2H), 3.59-3.46 (m, 3H), 3.31-3.24 (m, 1H), 2.97-2.92 (m, 1H), 2.64-2.51 (m, 1H), 2.37-2.17 (m, 2H), 1.69-1.55 (m, 3H), 1.46 (d, J=6.4 Hz, 3H), 1.26-1.07 (m, 3H). ¹⁹F NMR (376 MHz, DMSO-d₆) δ −61.34.

### 30C:

LC-MS (ESI): R_{T}=3.796 min, mass calcd. for C₂₉H₃₀ClF₃N₆O₃S 634.2 m/z found 635.3 [M+H]⁺. Chiral analysis (Column: Chiralpak IF 5 μm 4.6*250 mm; Mobile Phase: ACN:IPA=50:50 at 1 mL/min; Temp: 30° C.; Wavelength: 254 nm, R_{T}=6.035 min). ¹H NMR (400 MHz, DMSO-d₆) δ 8.81 (br s, 1H), 8.15-8.01 (m, 2H), 7.91 (s, 1H), 7.84 (d, J=8.4 Hz, 1H), 7.80-7.78 (m, 1H), 5.94-5.74 (m, 1H), 5.53-5.17 (m, 1H), 4.60-4.36 (m, 2H), 4.23-4.06 (m, 1H), 3.90-3.73 (m, 3H), 3.54-3.48 (m, 1H), 3.31-3.15 (m, 2H), 2.97-2.92 (m, 1H), 2.65-2.51 (m, 1H), 2.37-2.18 (m, 2H), 1.73-1.53 (m, 3H), 1.31 (d, J=6.4 Hz, 3H), 1.24-1.07 (m, 3H). ¹⁹F NMR (376 MHz, DMSO-d₆) δ −61.33.

### 30D:

LC-MS (ESI): R_{T}=3.870 min, mass calcd. for C₂₉H₃₀ClF₃N₆O₃S 634.2 m/z found 635.2 [M+H]⁺. Chiral analysis (Column: Chiralpak IF 5 μm 4.6*250 mm; Mobile Phase: ACN:IPA=50:50 at 1 mL/min; Temp: 30° C.; Wavelength: 254 nm, R_{T}=8.746 min). ¹H NMR (400 MHz, DMSO-d₆) δ 8.74 (br s, 1H), 8.14-7.98 (m, 2H), 7.91 (s, 1H), 7.84 (d, J=8.4 Hz, 1H), 7.79 (d, J=8.0 Hz, 1H), 6.03-5.70 (m, 1H), 5.58-5.16 (m, 1H), 4.62-4.37 (m, 2H), 4.24-4.08 (m, 1H), 3.89-3.73 (m, 2H), 3.60-3.45 (m, 3H), 3.31-3.24 (m, 1H), 2.97-2.92 (m, 1H), 2.65-2.51 (m, 1H), 2.38-2.17 (m, 2H), 1.72-1.55 (m, 3H), 1.46 (d, J=6.4 Hz, 3H), 1.28-1.06 (m, 3H). ¹⁹F NMR (376 MHz, DMSO-d₆) δ −61.34.

### Compounds 31A and 31B

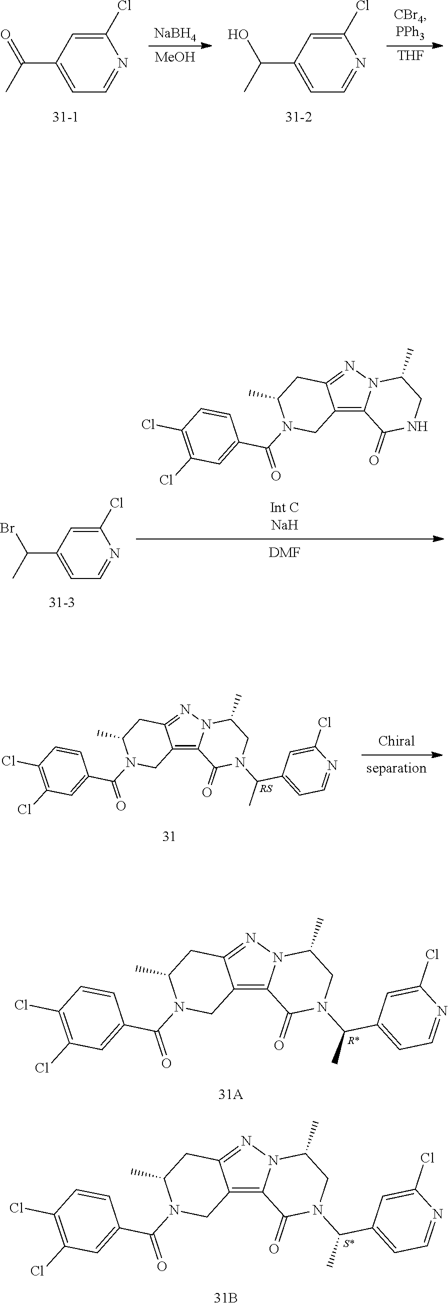

### Intermediate 31-2

### 1-(2-Chloropyridin-4-yl)ethanol

To the solution of 1-(2-chloropyridin-4-yl)ethanone 31-1 (2.00 g, 12.9 mmol) in methanol (15 mL) was added sodium borohydride (1.00 g, 26.4 mmol) at 0° C. The mixture was stirred at 0° C. for 0.5 hour, then warm to room temperature and stirred for another 1 hour, then the mixture was concentrated in vacuum to give a residue. The residue was poured into water (10 mL) and extracted with ethyl acetate (10 mL) twice. The combined organic layers were washed with brine (10 mL) twice, dried over Na₂SO₄₍ₛ₎ and filtered. The filtrate was concentrated to give the title compound (1.60 g, 90% purity from ¹H NMR, 71% yield) as yellow oil. LC-MS (ESI): R_{T}=1.15 min, mass calcd. for C₇H₈ClNO 157.03, m/z found 158.0 [M+H]⁺.

### Intermediate 31-3

### 4-(1-Bromoethyl)-2-chloropyridine

To a solution of 1-(2-chloropyridin-4-yl)ethanol 31-2 (1.60 g, 90% purity, 9.14 mmol) in tetrahydrofuran (15 mL) were added triphenylphosphine (4.80 g, 18.3 mmol) and perbromomethane (4.50 g, 13.6 mmol) at 0° C. After stirred at room temperature for 1 hour, the reaction mixture was concentrated and purified by silica gel column chromatography (petroleum ether:ethyl acetate=10:1 to 2:1) to give the compound (2.00 g, 98% purity from LCMS, 97% yield) as yellow oil. LC-MS (ESI): R_{T}=1.57 min, mass calcd. for C₇H₇BrClN 218.95, m/z found 222.0 [M+H]⁺.

### Compound 31

### (3R,7R)-9-(1-(2-Chloropyridin-4-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (350 mg, 100% purity, 0.89 mmol) in N,N-dimethylformamide (5 mL) was added 60% wt. sodium hydride in mineral oil (150 mg, 3.75 mmol). After stirred at 0° C. for 0.5 hour, 4-(1-bromoethyl)-2-chloropyridine 31-3 (300 mg, 95% purity, 1.29 mmol) was added into the mixture. After stirred at 0° C. for 1 hour, the mixture was diluted with water (50 mL) and extracted with ethyl acetate (30 mL) twice. The combined organic layers were washed with brine (30 mL), dried over Na₂SO₄₍ₛ₎ and filtered. The filtrate was concentrated in vacuum to give the crude compound, which was purified by silica gel column chromatography (petroleum ether:ethyl acetate=1:1) to give the title compound (350 mg, 95% purity from ¹H NMR, 70.1% yield) as white solids. LC-MS (ESI): R_{T}=1.67 min, mass calcd. for C₂₅H₂₄Cl₃N₅O₂ 531.1, m/z found 532.5 [M+H]⁺.

### Compounds 31A and 31B

### (3R,7R)-9-((R*)-1-(2-chloropyridin-4-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (31A) and (3R,7R)-9-((S*)-1-(2-chloropyridin-4-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (31B)

A racemic mixture of (3R,7R)-9-(1-(2-chloropyridin-4-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 31 (350 mg, 95% purity, 0.62 mmol) was separated by chiral Prep. HPLC (separation condition: Column: Chiralpak IE 5 μm 20*250 mm; Mobile Phase: ACN:IPA=70:30 at 25 mL/min; Temp: 30° C.; Wavelength: 254 nm) to give the title compounds 31A (100 mg, 97.4% purity from LCMS, 29.3% yield, 99.9% stereopure) and 31B (110 mg, 98.1% purity from LCMS, 32.5% yield, 99.9% stereopure) as white solids.

### 31A:

LC-MS (ESI): R_{T}=2.821 min, mass calcd. for C₂₅H₂₄Cl₃N₅O₂ 531.1, m/z found 532.2 [M+H]⁺. Chiral analysis (Column: Chiralpak IE 5 m 4.6*250 mm; Mobile Phase: ACN:IPA=70:30 at 1 mL/min; Col. Temp: 30° C.; Wavelength: 254 nm; Rt=7.032 min). ¹H NMR (400 MHz, CDCl₃) δ 8.40 (d, J=5.2 Hz, 1H), 7.54-7.50 (m, 2H), 7.36-7.32 (m, 1H), 7.29-7.25 (m, 1H), 7.21-7.16 (m, 1H), 6.01 (br s, 1H), 5.75-5.36 (m, 1H), 4.84-4.28 (m, 3H), 3.69-3.58 (m, 1H), 3.13-2.94 (m, 2H), 2.75-2.65 (m, 1H), 1.60-1.58 (m, 3H), 1.37 (d, J=6.8 Hz, 3H), 1.29-1.23 (m, 3H).

### 31B:

LC-MS (ESI): R_{T}=2.831 min, mass calcd. for C₂₅H₂₄Cl₃N₅O₂ 531.1, m/z found 532.2 [M+H]⁺. Chiral analysis (Column: Chiralpak IE 5 m 4.6*250 mm; Mobile Phase: ACN:IPA=70:30 at 1 mL/min; Col. Temp: 30° C.; Wavelength: 254 nm; Rt=8.348 min). ¹H NMR (400 MHz, CDCl₃) δ 8.38 (d, J=5.2 Hz, 1H), 7.54-7.50 (m, 2H), 7.28-7.22 (m, 2H), 7.18-7.13 (m, 1H), 6.01 (br s, 1H), 5.74-5.29 (m, 1H), 4.86-4.29 (m, 3H), 3.36-3.25 (m, 2H), 3.15-2.96 (m, 1H), 2.70-2.66 (m, 1H), 1.61-1.56 (m, 6H), 1.31-1.20 (m, 3H).

### Compounds 32A and 32B

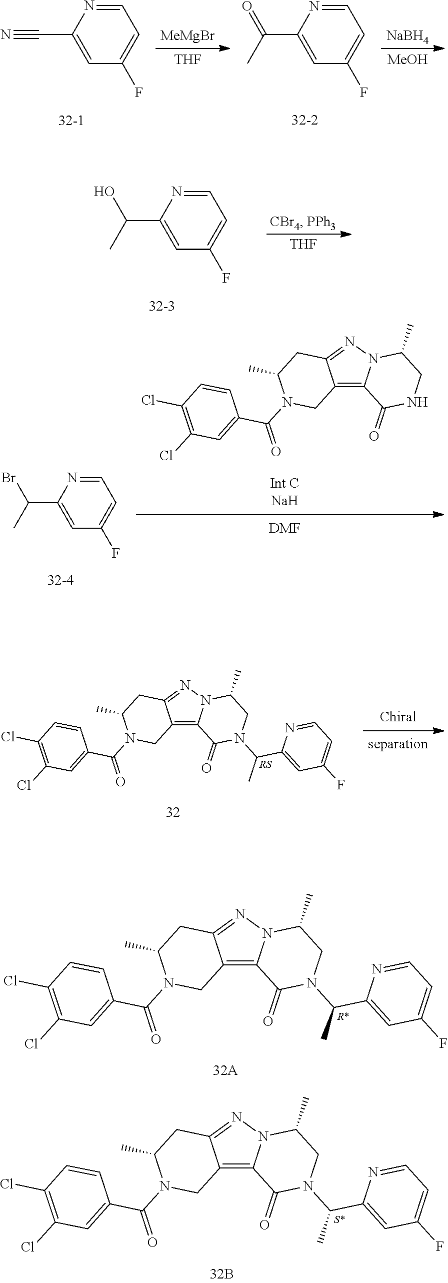

### Intermediate 32-2

### 1-(4-Fluoropyridin-2-yl)ethanone

To the solution of 4-fluoropicolinonitrile 32-1 (200 mg, 1.64 mmol) in tetrahydrofuran (4 mL) was added 3 M methylmagnesium bromide in 2-methyltetrahydrofuran (1.6 mL, 4.8 mmol) at 0° C. After stirred at 0° C. for 30 minutes, the mixture was quenched with ammonium chloride aqueous solution (30 mL) and extracted with ethyl acetate (50 mL) twice. The combined organic layers were washed with brine (40 mL), dried over Na₂SO₄₍ₛ₎ and filtered. The filtrate was concentrated to give the crude title compound (200 mg, 72% purity from LCMS, 63.2% yield) as yellow oil. LC-MS (ESI): R_{T}=1.28 min, mass calcd. for C₇H₆FNO 139.0, m/z found 140.0 [M+H]⁺. ¹H NMR (400 MHz, CDCl₃) δ 8.66 (dd, J=8.4 and 5.6 Hz, 1H), 7.76 (dd, J=9.2 and 2.8 Hz, 1H), 7.22-7.18 (m, 1H), 2.73 (s, 3H).

### Intermediate 32-3

### 1-(4-Fluoropyridin-2-yl)ethanol

To the solution of 1-(4-fluoropyridin-2-yl)ethanone 32-2 (200 mg, 72% purity, 1.04 mmol) in methanol (2 mL) was added sodium borohydride (40 mg, 1.06 mmol) at 0° C. After stirred at 0° C. for 30 minutes, the mixture was quenched with ammonium chloride aqueous solution (10 mL) and extracted with dichloromethane (30 mL) twice. The combined organic layers were washed with brine (20 mL), dried over Na₂SO₄₍ₛ₎ and filtered. The filtrate was concentrated to give the crude title compound (140 mg, 68% purity, 65.2% yield) as yellow oil. LC-MS (ESI): R_{T}=0.95 min, mass calcd. for C₇H₈FNO 141.1, m/z found 142.3 [M+H]⁺. ¹H NMR (400 MHz, CDCl₃) δ 8.51 (dd, J=8.0, 5.6 Hz, 1H), 7.04 (dd, J=9.6, 2.4 Hz, 1H), 6.97-6.93 (m, 1H), 4.89 (q, J=6.4 Hz, 1H), 3.91 (s, 1H), 1.51 (d, J=6.4 Hz, 3H).

### Intermediate 32-4

### 2-(1-Bromoethyl)-4-fluoropyridine

To a solution of 1-(4-fluoropyridin-2-yl)ethanol 32-3 (140 mg, 68% purity, 0.67 mmol) in tetrahydrofuran (2 mL) were added triphenylphosphine (340 mg, 1.30 mmol) and perbromomethane (340 mg, 1.03 mmol) at 0° C. After stirred at room temperature for 1 hour, the reaction mixture was concentrated and purified by silica gel column chromatography (petroleum ether:ethyl acetate=3:1) to give the title compound (70 mg, 100% purity, 50.9% yield) as yellow oil. LC-MS (ESI): R_{T}=1.50 min, mass calcd. for C₇H₇BrFN 203.0, m/z found 204.2 [M+H]⁺. ¹H NMR (400 MHz, CDCl₃) δ 8.54 (dd, J=8.4, 5.6 Hz, 1H), 7.21 (dd, J=9.6, 2.4 Hz, 1H), 6.97-6.93 (m, 1H), 5.22-5.17 (m, 1H), 2.06 (d, J=7.2 Hz, 3H).

### Compound 32

### (3R,7R)-2-(3,4-Dichlorobenzoyl)-9-(1-(4-fluoropyridin-2-yl)ethyl)-3,7-dimethyl-1,2,3,4,8,9-hexahydropyrido[4′,3′:3,4]pyrazolo[1,5-a]pyrazin-10(7H)-one

To the solution of (3R,7R)-2-(3,4-dichlorobenzoyl)-3,7-dimethyl-1,2,3,4,8,9-hexa hydropyrido[4′,3′:3,4]pyrazolo[1,5-a]pyrazin-10(7H)-one Int C (120 mg, 100% purity, 0.31 mmol) in N,N-dimethylformamide (2 mL) was added 60% wt. sodium hydride in mineral oil (35 mg, 0.88 mmol) at 0° C. The mixture was stirred at 0° C. for 30 minutes, then 2-(1-bromoethyl)-4-fluoropyridine 32-4 (90 mg, 100% purity, 0.44 mmol) was added to the mixture. After stirred at room temperature for 1.5 hours, the mixture was quenched with water (20 mL) and extracted with ethyl acetate (50 mL) twice. The combined organic layers were washed with brine (40 mL), dried over Na₂SO₄₍ₛ₎ and filtered. The filtrate was concentrated and purified by C18 column (acetonitrile:water=5% to 95%) to give the title compound (140 mg, 100% purity from LCMS, 88.8% yield) as yellow solids. LC-MS (ESI): R_{T}=1.66 min, mass calcd. for C₂₅H₂₄Cl₂FN₅O₂ 515.1, m/z found 516.2 [M+H]⁺.

### Compounds 32A and 32B

### (3R,7R)-2-(3,4-dichlorobenzoyl)-9-((R*)-1-(4-fluoropyridin-2-yl)ethyl)-3,7-dimethyl-1,2,3,4,8,9-hexahydropyrido[4′,3′:3,4]pyrazolo[1,5-a]pyrazin-10(7H)-one (32A) and (3R,7R)-2-(3,4-dichlorobenzoyl)-9-((S*)-1-(4-fluoropyridin-2-yl)ethyl)-3,7-dimethyl-1,2,3,4,8,9-hexahydropyrido[4′,3′:3,4]pyrazolo[1,5-a]pyrazin-10(7H)-one (32B)

The racemate of (3R,7R)-2-(3,4-dichlorobenzoyl)-9-(1-(4-fluoropyridin-2-yl)ethyl)-3,7-dimethyl-1,2,3,4,8,9-hexahydropyrido[4′,3′:3,4]pyrazolo[1,5-a]pyrazin-10(7H)-one 32 (140 mg, 100% purity, 0.27 mmol) was separated by chiral Prep. HPLC (separation condition: Column: Chiralpak IB N-5 5 m 30*250 mm, Mobile Phase: Hex:EtOH=70:30 at 30 mL/min; Temp: 30° C.; Wavelength: 254 nm), then further purified with Prep. HPLC (Column: X-bridge C18; column size: (5 m 19*150 mm); Mobile Phase A: water (+0.1% ammonium bicarbonate), Mobile Phase B: acetonitrile, Gradient: 5-95% (% B), UV: 214 nm, 15 mL/min) to give the title compounds 32A (40 mg, 99.7% purity, 28.5% yield, 100% stereopure) as white solids and 32B (40 mg, 99.9% purity, 28.5% yield, 100% stereopure) as white solids.

### 32A:

LC-MS (ESI): R_{T}=2.981 min, mass calcd. for C₂₅H₂₄Cl₂FN₅O₂ 515.1, m/z found 516.2 [M+H]⁺. Chiral HPLC (Column: Chiralpak IB N-5 5 m 4.6*250 mm; Mobile Phase: Hex:EtOH=70:30 at 1 mL/min; Temp: 30° C.; Wavelength: 230 nm, R_{T}=9.107 min). ¹H NMR (400 MHz, CDCl₃) δ 8.54-8.51 (m, 1H), 7.55-7.47 (m, 2H), 7.26-7.24 (m, 1H), 7.14-7.02 (m, 1H), 6.98-6.94 (m, 1H), 5.98 (s, 1H), 5.71-5.42 (m, 1H), 4.84-4.22 (m, 3H), 3.73-3.63 (m, 1H), 3.55-3.44 (m, 1H), 3.19-2.89 (m, 1H), 2.67 (d, J=14.4 Hz, 1H), 1.63 (d, J=6.4 Hz, 3H), 1.57-1.56 (m, 3H), 1.33-1.19 (m, 3H). ¹⁹F NMR (376 MHz, CDCl₃) δ −101.3.

### 32B:

LC-MS (ESI): R_{T}=2.997 min, mass calcd. for C₂₅H₂₄Cl₂FN₅O₂ 515.1, m/z found 516.2 [M+H]⁺. Chiral HPLC (Column: Chiralpak IB N-5 5 m 4.6*250 mm; Mobile Phase: Hex:EtOH=70:30 at 1 mL/min; Temp: 30° C.; Wavelength: 230 nm, R_{T}=10.576 min). ¹H NMR (400 MHz, CDCl₃) δ 8.56-8.53 (m, 1H), 7.56-7.47 (m, 2H), 7.26-7.23 (m, 1H), 7.17-7.06 (m, 1H), 6.99-6.95 (m, 1H), 5.97 (s, 1H), 5.71-5.42 (m, 1H), 4.80-4.23 (m, 3H), 3.81 (d, J=11.2 Hz, 1H), 3.52-3.47 (m, 1H), 3.17-2.88 (m, 1H), 2.66 (d, J=15.2 Hz, 1H), 1.62 (d, J=6.0 Hz, 3H), 1.35-1.20 (m, 6H). ¹⁹F NMR (376 MHz, CDCl₃) δ −101.4.

### Compounds 33A and 33B

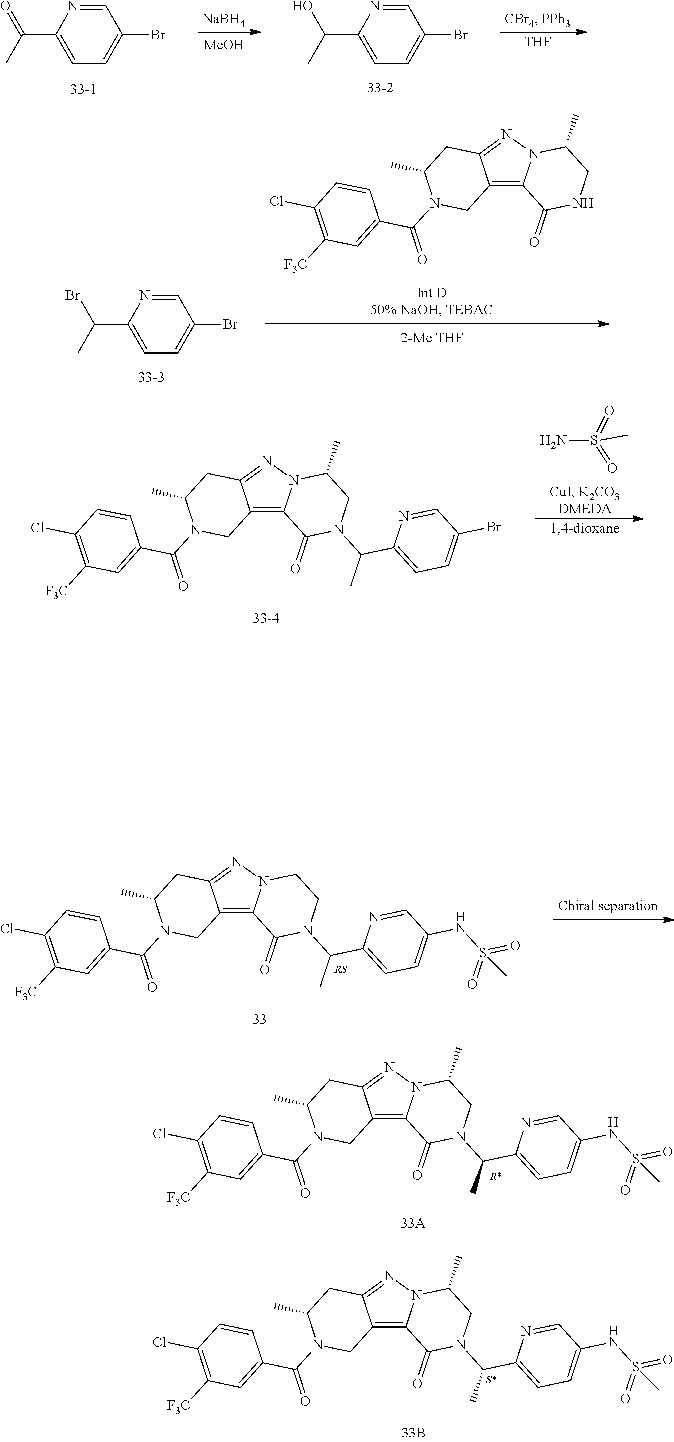

### Intermediate 33-2

### 1-(5-Bromopyridin-2-yl)ethan-1-ol

1-(5-Bromopyridin-2-yl)ethan-1-one 33-1 (2.0 g, 10.00 mmol) was dissolved in methanol (40 mL) and stirred at 0° C. Sodium borohydride (300 mg, 7.93 mmol) was added into the solution and stirred at 0° C. for 0.5 hour. Water (30 mL) was added into the reaction solution, and stirred for 0.5 hour. Then methanol was evaporated, extracted with dichloromethane (30 mL) for three times, the organic layers were combined, followed by dried over Na₂SO₄₍ₛ₎ and filtered. The solvent was evaporated to obtain the title compound (2.0 g, purity 87% from LCMS, 86% yield) as colorless oil. LC-MS (ESI): R_{T}=1.13 min, mass calcd. for C₇H₈BrNO 201.0 m/z found 201.9 [M+H]⁺.

### Intermediate 33-3

### 5-Bromo-2-(1-bromoethyl)pyridine

To a solution of 1-(5-bromopyridin-2-yl)ethan-1-ol 33-2 (1.0 g, 87% purity, 4.31 mmol) in tetrahydrofuran (20 mL) were added triphenylphosphine (2.3 g, 8.77 mmol) and carbon tetrabromide (2.2 g, 6.63 mmol) at 0° C. After stirred at room temperature for 3 hours, the mixture was filtered. The filtrate was concentrated and purified by column chromatography on silica gel (petroleum ether:acetone=10:1) to give the title compound (1.3 g, 80% purity from ¹H NMR, 91% yield) as yellow oil. ¹H NMR (400 MHz, CDCl₃) δ 8.62 (d, J=2.4 Hz, 1H), 7.81 (dd, J₁=8.4 Hz, J₂=2.0 Hz, 1H), 7.36 (d, J=8.4 Hz, 1H), 5.20 (q, J=7.2 Hz, 1H), 2.05 (d, J=7.2 Hz, 3H).

### Intermediate 33-4

### (3R,7R)-9-(1-(5-Bromopyridin-2-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one Int D (800 mg, 90% purity, 1.69 mmol), 5-bromo-2-(1-bromoethyl)pyridine 33-3 (838 mg, 80% purity, 2.53 mmol) and N-benzyl-N,N-diethylethanaminium chloride (58 mg, 0.26 mmol) in 2-methyltetrahydrofuran (10 mL) was added 50% sodium hydroxide solution (4 mL) at 0° C. Then the reaction mixture was stirred at room temperature for 8 hours. The reaction mixture was quenched with water (30 mL) and extracted with ethyl acetate (30 mL) twice. The combined organic layers were dried over Na₂SO₄₍ₛ₎ and filtered. The filtrate was concentrated in vacuum to give a residue, which was purified by silica gel column chromatography (dichloromethane:methanol=1:0 to 20:1) to afford product (690 mg, 100% purity from LCMS, 67% of yield) as white solids. LC-MS (ESI): R_{T}=1.299 min, mass calcd. for C₂₆H₂₄BrClF₃N₅O₂ 609.1 m/z found 610.0[M+H]⁺.

### Compound 33

### N-(6-(1-((3R,7R)-2-(4-Chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl) pyridin-3-yl)methanesulfonamide

To a solution of (3R,7R)-9-(1-(5-bromopyridin-2-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one 33-4 (300 mg, purity 100%, 0.49 mmol) in 1,4-dioxane (6 ml) were added potassium carbonate (136 mg, 0.98 mmol), N,N-dimethylethane-1,2-diamine (43 mg, 0.49 mmol), cuprous iodide (94 mg, 0.49 mmol) and methanesulfonamide (93 mg, 0.98 mmol). The mixture was stirred at 110° C. for 16 hours. After cooled to room temperature, water (20 ml) was added to the reaction mixture and the layers were separated. The aqueous layer was extracted with ethyl acetate (20 ml) and the organic fractions were dried over Na₂SO₄₍ₛ₎, filtered and evaporated in vacuum. The resulting residue was purified by flash chromatography (dichloromethane:methanol=1:0 to 20:1) to give the title compound (200 mg, 95% purity from LCMS, 62% yield) as white solids. LC-MS (ESI): R_{T}=1.726 min, mass calcd. for C₂₇H₂₈ClF₃N₆O₄S 624.2 m/z found 625.1[M+H]⁺.

### Compounds 33A and 33B

### N-(6-((R*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)pyridin-3-yl)methanesulfonamide (33A) and N-(6-((S*)-1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)pyridin-3-yl)methanesulfonamide (33B)

A racemic mixture of N-(6-(1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)pyridin-3-yl)methanesulfonamide 33 (200 mg, 95% purity, 0.304 mmol) was separated by chiral HPLC (Column: Chiralpak IE 5 m, 20*250 mm; Mobile Phase: MeOH:EtOH=50:50 at 30 g/min; Temp: 30° C.; Wavelength: 254 nm) to afford the title compounds 33A (30 mg, 15% yield, 96.6% purity from LCMS, 100% stereopure) as white solids and 33B (40 mg, 21% yield, 99.3% purity from LCMS, 100% stereopure) as white solids.

### 33A:

LC-MS (ESI): R_{T}=3.049 min, mass calcd. for C₂₇H₂₈ClF₃N₆O₄S 624.2 m/z found 625.1 [M+H]⁺. Chiral analysis (Chiralpak IE 5 m 4.6*250 mm; Mobile Phase: MeOH:EtOH=50:50 at 1 mL/min; Temp: 30° C.; Wavelength: 254 nm; R_{T}=5.647 min). ¹H NMR (400 MHz, DMSO-d₆) δ 9.99 (s, 1H), 8.39 (s, 1H), 7.93-7.90 (m, 1H), 7.85-7.76 (m, 2H), 7.65-7.57 (m, 1H), 7.44-7.33 (m, 1H), 5.90-5.72 (m, 1H), 5.51-5.14 (m, 1H), 4.55-4.37 (m, 2H), 4.25-4.09 (m, 1H), 3.87-3.73 (m, 1H), 3.27-3.21 (m, 1H), 3.04 (s, 3H), 2.98-2.90 (m, 1H), 2.59-2.51 (m, 1H), 1.60-1.46 (m, 3H), 1.26-1.08 (m, 6H). ¹⁹F NMR (376 MHz, DMSO-d₆) δ −61.33.

### 33B:

LC-MS (ESI): R_{T}=3.506 min, mass calcd. for C₂₇H₂₈ClF₃N₆O₄S 624.2 m/z found 625.2 [M+H]⁺. Chiral analysis (Chiralpak IE 5 m 4.6*250 mm; Mobile Phase: MeOH:EtOH=50:50 at 1 mL/min; Temp: 30° C.; Wavelength: 254 nm; R_{T}=6.303 min). ¹H NMR (400 MHz, DMSO-d₆) δ 9.97 (s, 1H), 8.39 (s, 1H), 7.93-7.89 (m, 1H), 7.85-7.78 (m, 2H), 7.58 (s, 1H), 7.44-7.32 (m, 1H), 5.91-5.68 (m, 1H), 5.51-5.20 (m, 1H), 4.58-4.41 (m, 2H), 4.22-4.10 (m, 1H), 3.62-3.44 (m, 2H), 3.04 (s, 3H), 2.99-2.89 (m, 1H), 2.60-2.51 (m, 1H), 1.55-1.43 (m, 6H), 1.25-1.07 (m, 3H). ¹⁹F NMR (376 MHz, DMSO-d₆) δ −61.34.

### Compounds 34A and 34B

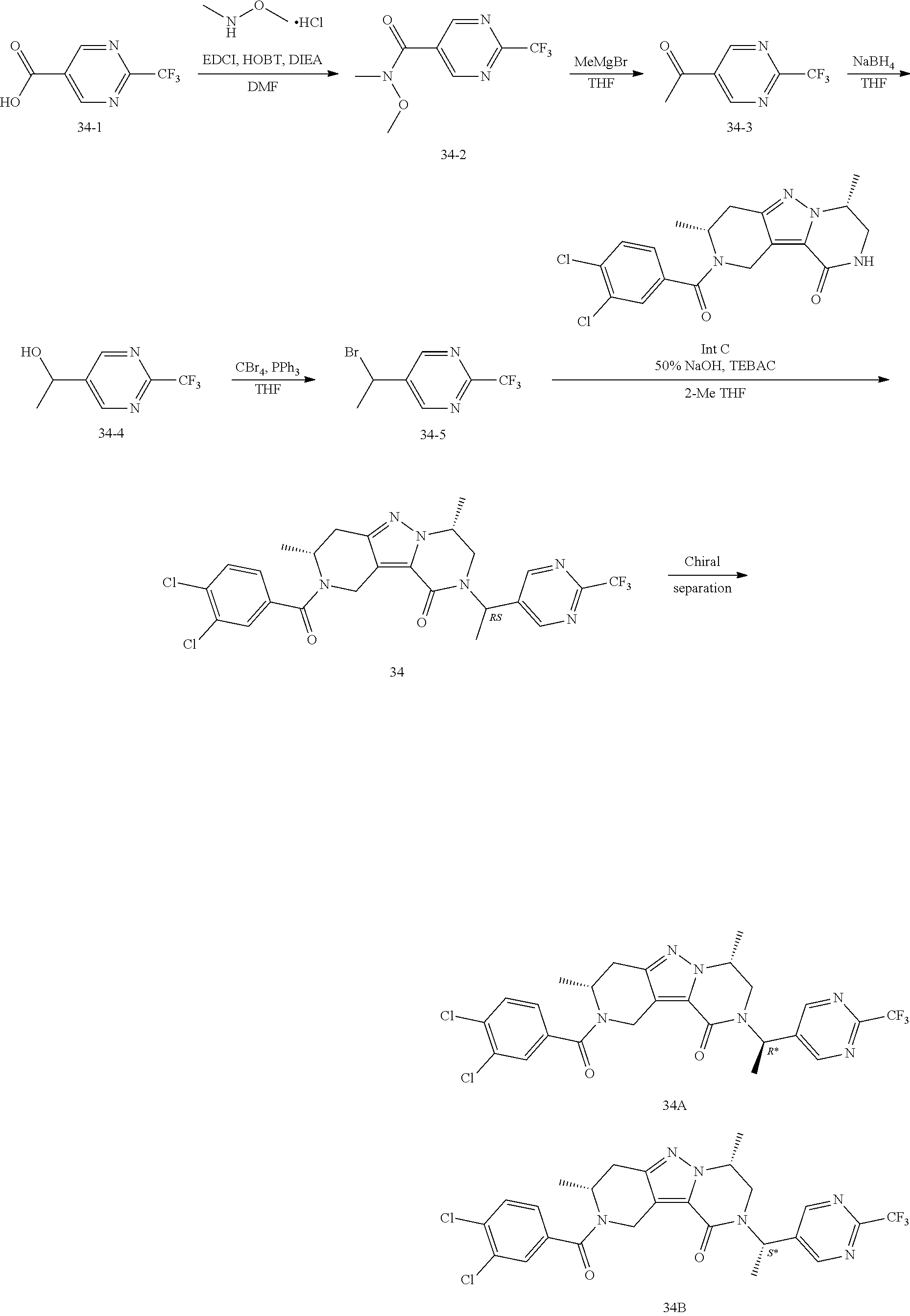

### Intermediate 34-2

### N-Methoxy-N-methyl-2-(trifluoromethyl)pyrimidine-5-carboxamide

To the solution of 2-(trifluoromethyl)pyrimidine-5-carboxylic acid 34-1 (5.0 g, 26.0 mmol) in N,N-dimethylformamide (55 mL) were added N,O-dimethylhydroxylamine hydrochloride (5.1 g, 52.3 mmol), benzotriazol-1-ol (7.1 g, 52.5 mmol), 1-ethyl-(3-(3-dimethylamino)propyl)-carbodiimide hydrochloride (10.1 g, 52.7 mmol) and N-ethyl-N-isopropylpropan-2-amine (20.5 g, 158.6 mmol) at 0° C. The mixture was stirred at room temperature for 16 hours. The mixture was diluted with water (100 mL), acidized with 0.5 M hydrochloric acid aqueous solution to pH˜5 and extracted with ethyl acetate (100 mL) twice. The combined organic layers were washed with brine (100 mL), dried over Na₂SO₄₍ₛ₎ and filtered. The filtrate was concentrated and purified by C18 column (acetonitrile:water=30% to 60%) to give the title compound (5 g, 100% purity from LCMS, 81.7% yield) as yellow solids. LC-MS (ESI): R_{T}=1.40 min, mass calcd. for C₈H₈F₃N₃O₂ 235.1, m/z found 236.1 [M+H]⁺. ¹H NMR (400 M Hz, CDCl₃) δ 9.24 (s, 2H), 3.62 (s, 3H), 3.44 (s, 3H).

### Intermediate 34-3

### 1-(2-(Trifluoromethyl)pyrimidin-5-yl)ethan-1-one

To a solution of N-Methoxy-N-methyl-2-(trifluoromethyl)pyrimidine-5-carboxamide 34-2 (5 g, 100% purity, 21.3 mmol) in tetrahydrofuran (50 mL) was added 1 M methylmagnesium bromide in 2-methyltetrahydrofuran (33.3 mL, 33.3 mmol) dropwise at 0° C. After being stirred at 0° C. for 2 hours, the reaction mixture was poured into saturated aqueous ammonium chloride solution (50 mL) and extracted with ethyl acetate (50 mL) twice. The combined organic layers were washed with brine (50 mL), dried over Na₂SO₄₍ₛ₎ and filtered. The filtrate was concentrated to give the title compound (4.5 g, 77% purity from LCMS, 85.7% yield) as yellow oil. LC-MS (ESI): R_{T}=1.36 min, mass calcd. for C₇H₅F₃N₂O 190.0, m/z found 189.0 [M−H]⁻.

### Intermediate 34-4

### 1-(2-(Trifluoromethyl)pyrimidin-5-yl)ethanol

To the solution of 1-(2-(trifluoromethyl)pyrimidin-5-yl)ethan-1-one 34-3 (4.5 g, 77% purity, 18.2 mmol) in tetrahydrofuran (50 mL) was added sodium borohydride (1.54 g, 40.7 mmol) at 0° C. After being stirred at 0° C. for 1 hour, the mixture was quenched with saturated aqueous ammonium chloride solution (50 mL) and extracted with ethyl acetate (50 mL). The organic layer was washed with water (50 mL) twice, dried over Na₂SO₄₍ₛ₎ and concentrated in vacuum to give a residue, which was purified by C18 (acetonitrile:water=40% to 65%) to give the desired product (2.3 g, 90% purity, 59.1% yield) as yellow oil. ¹H NMR (400 M Hz, CDCl₃) δ 8.92 (s, 2H), 5.15-5.06 (m, 1H), 1.62-1.61 (m, 3H).

### Intermediate 34-5

### 5-(1-bromoethyl)-2-(trifluoromethyl)pyrimidine

To a solution of 1-(2-(trifluoromethyl)pyrimidin-5-yl)ethanol 34-4 (2 g, 90% purity, 9.37 mmol) in tetrahydrofuran (20 mL) were added triphenylphosphine (5 g, 19.1 mmol) and perbromomethane (5 g, 15.1 mmol) at 0° C. After being stirred at 25° C. for 0.5 hour, the mixture was filtered. The filtrate was concentrated in vacuum to give a residue, which was purified by silica gel column chromatography (petroleum ether:ethyl acetate=100:1) to give the title compound (1 g, 90% purity from ¹H NMR, 37.7% yield) as yellow oil. ¹H NMR (400 M Hz, CDCl₃) δ 8.96 (s, 2H), 5.18-5.17 (m, 1H), 2.13-2.11 (m, 3H).

### Compound 34

### (3R,7R)-2-(3,4-Dichlorobenzoyl)-3,7-dimethyl-9-(1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (200 mg, 90% purity, 0.458 mmol) in 2-methyltetrahydrofuran (4 mL) and 50% wt. sodium hydroxide in water (2 mL, 50.0 mmol) were added 5-(1-bromoethyl)-2-(trifluoromethyl)pyrimidine 34-5 (200 mg, 90% purity, 0.706 mmol) and benzyltriethylammonium chloride (40 mg, 0.176 mmol) at room temperature. After stirred at room temperature for 2 hours, the reaction mixture was quenched with water (10 mL) and extracted with ethyl acetate (10 mL) twice. The combined organic layers were washed with brine (10 mL), dried over Na₂SO₄₍ₛ₎ and filtered. The filtrate was concentrated and purified by C18 column (acetonitrile:water=50% to 60%) to give the title compound (160 mg, 100% purity, 61.6% yield) as white solids. LC-MS (ESI): R_{T}=1.69 min, mass calcd. for C₂₅H₂₃Cl₂F₃N₆O₂ 566.1, m/z found 567.3 [M+H]⁺.

### Compounds 34A and 34B

### (3R,7R)-2-(3,4-dichlorobenzoyl)-3,7-dimethyl-9-((R*)-1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one (34A) and (3R,7R)-2-(3,4-dichlorobenzoyl)-3,7-dimethyl-9-((S*)-1-(2-(trifluoromethyl)pyrimidin-5-yl)ethyl)-1,2,3,4,8,9-hexahydropyrido[4′,3′:3,4]pyrazolo[1,5-a]pyrazin-10(7H)-one (34B)

The racemic mixture of (3R,7R)-2-(3,4-dichlorobenzoyl)-3,7-dimethyl-9-(1-(2-(trifluoromethyl)pyrimidin-5-yl)ethyl)-1,2,3,4,8,9-hexahydropyrido[4′,3′:3,4]pyrazolo [1,5-a]pyrazin-10(7H)-one 34 (130 mg, 100% purity, 0.229 mmol) was separated by chiral Prep. (Column: Chiralpak IE 5 μm 30*250 mm; Mobile Phase: Hex:EtOH=30:70 at 25 mL/min; Temp: 30° C.; Wavelength: 254 nm) to give the title compounds 34A (19.3 mg, 99.6% purity, 14.8% yield, 100% stereopure) as white solids and 34B (19.4 mg, 99.2% purity, 14.8% yield, 100% stereopure) as white solids.

### 34A:

LC-MS (ESI): R_{T}=3.493 min, mass calcd. for C₂₅H₂₃Cl₂F₃N₆O₂ 566.1, m/z found 567.2 [M+H]⁺. Chiral analysis (Column: Chiralpak IE 5 μm 4.6*250 mm; Mobile Phase: HeX:EtOH=30:70 at 1 mL/min; Temp: 30° C.; Wavelength: 254 nm, R_{T}=8.200 min). ¹H NMR (400 MHz, CDCl₃) δ 8.92 (s, 2H), 7.57-7.47 (m, 2H), 7.28-7.23 (m, 1H), 6.25-6.08 (m, 1H), 5.76-5.30 (m, 1H), 4.90-4.21 (m, 3H), 3.80-3.67 (m, 1H), 3.15-2.94 (m, 2H), 2.74-2.60 (m, 1H), 1.72-1.71 (m, 3H), 1.41 (d, J=6.4 Hz, 3H), 1.33-1.19 (m, 3H). ¹⁹F NMR (376 MHz, CDCl₃) δ −70.27.

### 34B:

LC-MS (ESI): R_{T}=3.486 min, mass calcd. for C₂₅H₂₃Cl₂F₃N₆O₂ 566.1, m/z found 567.2[M+H]⁺. Chiral analysis (Column: Chiralpak IE 5 μm 4.6*250 mm; Mobile Phase: HeX:EtOH=30:70 at 1 mL/min; Temp: 30° C.; Wavelength: 254 nm, R_{T}=14.055 min). ¹H NMR (400 MHz, CDCl₃) δ 8.89 (s, 2H), 7.58-7.47 (m, 2H), 7.31-7.22 (m, 1H), 6.28-6.03 (m, 1H), 5.79-5.24 (m, 1H), 4.95-4.20 (m, 3H), 3.46-3.38 (m, 1H), 3.34-3.28 (m, 1H), 3.20-2.95 (m, 1H), 2.79-2.60 (m, 1H), 1.73-1.71 (m, 3H), 1.61-1.59 (m, 3H), 1.32-1.21 (m, 3H). ¹⁹F NMR (376 MHz, CDCl₃) δ −70.26.

### Compounds 35A and 35B

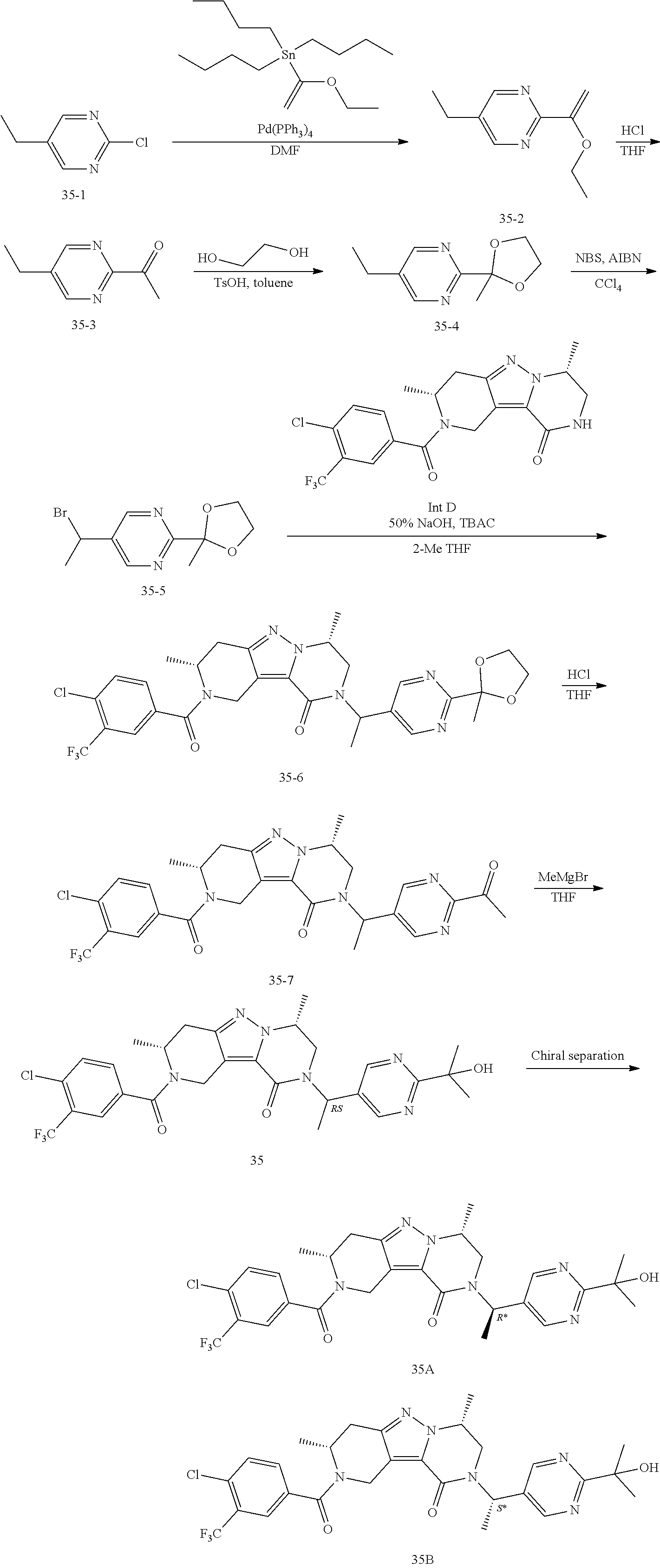

### Intermediate 35-2

### 2-(1-ethoxyvinyl)-5-ethylpyrimidine

To a solution of 2-chloro-5-ethylpyrimidine 35-1 (10 μg, 70.1 mmol), tetrakis(triphenylphosphoranyl)palladium (1.1 g, 0.949 mmol) in N,N-dimethylformamide (100 mL) was added tributyl(1-ethoxyvinyl)stannane (28 mL, 82.9 mmol). The reaction was heated to 110° C. for 12 hours. After cooled to room temperature, the reaction mixture was quenched with saturated potassium fluoride (100 mL). The mixture was stirred at room temperature for 2 hours and extracted with ethyl acetate (200 mL) for three times. The combined organic layers were washed with brine (200 mL), dried over Na₂SO₄₍ₛ₎ and filtered. The filtrate was concentrated and purified by silica gel column chromatography (petroleum ether:ethyl acetate=30:1 to 10:1) to give the title compound (8 g, 98% purity, 63% yield) as yellow oil. LC-MS (ESI): R_{T}=1.267 min, mass calcd. for C₁₀H₁₄N₂O₂178.1, m/z found 179.2 [M+H]⁺.

### Intermediate 35-3

### 1-(5-Ethylpyrimidin-2-yl)ethan-1-one

To a solution of 2-(1-ethoxyvinyl)-5-ethylpyrimidine 35-2 (8 g, 98% purity, 44 mmol) in tetrahydrofuran (40 mL) was added 1 M hydrochloric acid in water (40 mL, 40 mmol) at 0° C. After stirred at 40° C. for 4 hours, the reaction mixture was extracted with ethyl acetate (10 mL) for three times. The combined organic layers were washed with brine (20 mL) twice, dried over Na₂SO₄₍ₛ₎ and filtered. The filtrate was concentrated to give the compound (6 g, 91.5% purity from LCMS, 83% yield) as yellow oil. LC-MS (ESI): R_{T}=0.792 min, mass calcd. for C₈H₁₀N₂O 150.1, m/z found 151.1 [M+H]⁺.

### Intermediate 35-4

### 5-Ethyl-2-(2-methyl-1,3-dioxolan-2-yl)pyrimidine

To a mixture of 1-(5-ethylpyrimidin-2-yl)ethan-1-one 35-3 (6 g, 91.5% purity, 36.6 mmol) and ethane-1,2-diol (8.1 mL, 145.3 mmol) in toluene (60 mL) was added 4-methylbenzenesulfonic acid (900 mg, 5.23 mmol). After stirred at 100° C. for 16 hours, the mixture was concentrated. The reaction mixture was quenched with water (70 mL) and extracted with ethyl acetate (70 mL) twice. The combined organic layers were washed with brine (70 mL), dried over Na₂SO₄₍ₛ₎ and filtered. The filtrate was concentrated and purified by C18 column (acetonitrile:water=50% to 60%) to give the title compound (3.8 g, 96.8% purity, 51.8% yield) as yellow oil. LC-MS (ESI): R_{T}=0.958 min, mass calcd. for C₁₀H₁₄N₂O₂ 194.1, m/z found 195.2 [M+H]⁺.

### Intermediate 35-5

### 5-(1-Bromoethyl)-2-(2-methyl-1,3-dioxolan-2-yl)pyrimidine

To a solution of ethyl-2-(2-methyl-1,3-dioxolan-2-yl)pyrimidine 35-4 (3.8 g, 96.8% purity, 18.91 mmol) and N-bromsuccinimide (4 g, 22.51 mmol) in carbon tetrachloride (40 mL) was added azodiisobutyronitrile (380 mg, 2.31 mmol) under nitrogen atmosphere. After stirred at 70° C. overnight, the reaction mixture was cooled down to room temperature. The reaction mixture was quenched with water (40 mL) and extracted with ethyl acetate (40 mL) twice. The combined organic layers were washed with brine (50 mL), dried over Na₂SO₄₍ₛ₎ and filtered. The filtrate was purified by silica gel column chromatography (petroleum ether:ethyl acetate=40:1) to give the title compound (3.8 g, 53% purity, 38.9% yield) as yellow solids. LC-MS (ESI): R_{T}=1.29 min, mass calcd. for C₁₀H₁₃BrN₂O₂ 272.2, m/z found 273.0 [M+H]⁺.

### Intermediate 35-6

### (3R,7R)-2-(4-Chloro-3-(trifluoromethyl)benzoyl)-3,7-dimethyl-9-(1-(2-(2-methyl-1,3-dioxolan-2-yl)pyrimidin-5-yl)ethyl)-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one Int D (1.5 g, 100% purity, 3.5 mmol) in 2-methyltetrahydrofuran (15 mL) and sodium hydroxide in water (15 mL, 50 mmol) were added 5-(1-bromoethyl)-2-(2-methyl-1,3-dioxolan-2-yl)pyrimidine 32-5 (2.2 g, 53% purity, 4.3 mmol) and benzyltriethylammonium chloride (90 mg, 0.395 mmol) at room temperature. The mixture was stirred at room temperature for 2 hours. The reaction mixture was quenched with water (30 mL) and extracted with ethyl acetate (30 mL) twice. The combined organic layers were washed with brine (30 mL), dried over Na₂SO₄₍ₛ₎ and filtered.

The filtrate was concentrated and purified by C18 column (acetonitrile:water=50% to 60%) to give the title compound (1.5 g, 80% purity, 55.2% yield) as white solids. LC-MS (ESI): R_{T}=1.48 min, mass calcd. for C₂₉H₃₀ClF₃N₆O₄ 618.2, m/z found 619.2 [M+H]⁺.

### Intermediate 35-7

### (3R,7R)-9-(1-(2-Acetylpyrimidin-5-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-9-(1-(2-(2-methyl-1,3-dioxolan-2-yl)pyrimidin-5-yl)ethyl)-1,2,3,4,8,9-hexahydropyrido[4′,3′:3,4]pyrazolo[1,5-a]pyrazin-10(7H)-one 35-6 (1.5 g, 80% purity, 1.94 mmol) in tetrahydrofuran (7.5 mL) was added hydrochloride (7.5 mL, 90 mmol) at room temperature. After stirred at 70° C. for 3 hours, the reaction mixture was quenched with saturated sodium bicarbonate to pH˜7. The reaction solution was extracted with dichloromethane (20 mL) three times. The combined organic layers were washed with brine (20 mL), dried over anhydrous Na₂SO₄₍ₛ₎ and filtered. The filtrate was concentrated and purified by C18 column (acetonitrile:water=50% to 70%) to give the title compound (1.2 g, 73% purity, 78.6% yield) as yellow oil. LC-MS (ESI): R_{T}=1.50 min, mass calcd. for C₂₇H₂₆ClF₃N₆O₃ 574.2, m/z found 575.0 [M+H]⁺.

### Compound 35

### (3R,7R)-2-(4-chloro-3-(trifluoromethyl)benzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one

To the solution of (3R,7R)-9-(1-(2-acetylpyrimidin-5-yl)ethyl)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one 35-7 (300 mg, 73% purity, 0.381 mmol) in tetrahydrofuran (3 mL) was added 1 M methylmagnesium bromide in tetrahydrofuran (0.4 mL, 0.4 mmol) at −78° C. The mixture was stirred at −20° C. for 3 hours, then was quenched with ammonium chloride aqueous solution (5 mL), extracted with ethyl acetate (10 mL) twice. The combined organic layers were washed with brine (15 mL), dried over Na₂SO₄₍ₛ₎ and filtered. The filtrate was concentrated and purified by C18 column (acetonitrile:water=45% to 60%) to give the title compound (100 mg, 97% purity, 43% yield) as yellow oil. LC-MS (ESI): R_{T}=2.59 min, mass calcd. for C₂₈H₃₀ClF₃N₆O₃ 590.2, m/z found 591.0 [M+H]⁺.

### Compounds 35A and 35B

### (3R,7R)-2-(4-chloro-3-(trifluoromethyl)benzoyl)-9-((R*)-1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one (35A) and (3R,7R)-2-(4-chloro-3-(trifluoromethyl)benzoyl)-9-((S*)-1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one (35B)

The racemate of (3R,7R)-2-(4-chloro-3-(trifluoromethyl)benzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one 35 (100 mg, 100% purity, 0.17 mmol) was separated by chiral Prep.HPLC (Column: Chiralpak IC 5 m 30*250 mm; Mobile Phase: MeOH:EtOH:DEA=50:50:0.2 at 30 mL/min; Temp: 30° C.; Wavelength: 254 nm) to give the title compounds 35A (34.5 mg, 97.2% purity, 33.5% yield, 98.5% stereopure) as white solids and 35B (53.0 mg, 99.8% purity, 52.9% yield, 99.9% stereopure) as white solids.

### 35A:

LC-MS (ESI): R_{T}=3.602 min, mass calcd. for C₂₈H₃₀ClF₃N₆O₃ 590.2, m/z found 591.3 [M+H]⁺. Chiral analysis: (Column: Chiralpak IC 5 m 4.6*250 mm; Mobile Phase: EtOH: EtOH:DEA=50:50:0.2 at 1 mL/min; Temp: 30° C.; Wavelength: 254 nm, R_{T}=7.092 min). ¹H NMR (400 MHz, CDCl₃) δ 8.75 (s, 2H), 7.79 (s, 1H), 7.59-7.52 (m, 2H), 6.31-5.45 (m, 2H), 4.85-4.24 (m, 4H), 3.83-3.57 (m, 1H), 3.22-2.95 (m, 2H), 2.78-2.61 (m, 1H), 1.67-1.66 (m, 3H), 1.59-1.51 (m, 6H), 1.39-1.34 (m, 3H), 1.28-1.21 (m, 3H). ¹⁹F NMR (376 MHz, CDCl₃) δ −62.82.

### 35B:

LC-MS (ESI): R_{T}=3.629 min, mass calcd. for C₂₈H₃₀ClF₃N₆O₃ 590.2, m/z found 591.4 [M+H]⁺. Chiral analysis: (Column: Chiralpak IC 5 m 4.6*250 mm; Mobile Phase: EtOH: EtOH:DEA=50:50:0.2 at 1 mL/min; Temp: 30° C.; Wavelength: 254 nm, R_{T}=10.562 min). H NMR (400 MHz, CDCl₃) δ 8.71 (s, 2H), 7.79 (s, 1H), 7.60-7.53 (m, 2H), 6.19-6.03 (m, 1H), 5.78-5.36 (m, 1H), 4.90-4.19 (m, 4H), 3.42-3.33 (m, 2H), 3.19-2.97 (m, 1H), 2.78-2.62 (m, 1H), 1.68-1.66 (m, 3H), 1.58-1.52 (m, 9H), 1.35-1.19 (m, 3H). ¹⁹F NMR (376 MHz, CDCl3) δ −62.83.

### Compounds 36A and 36B

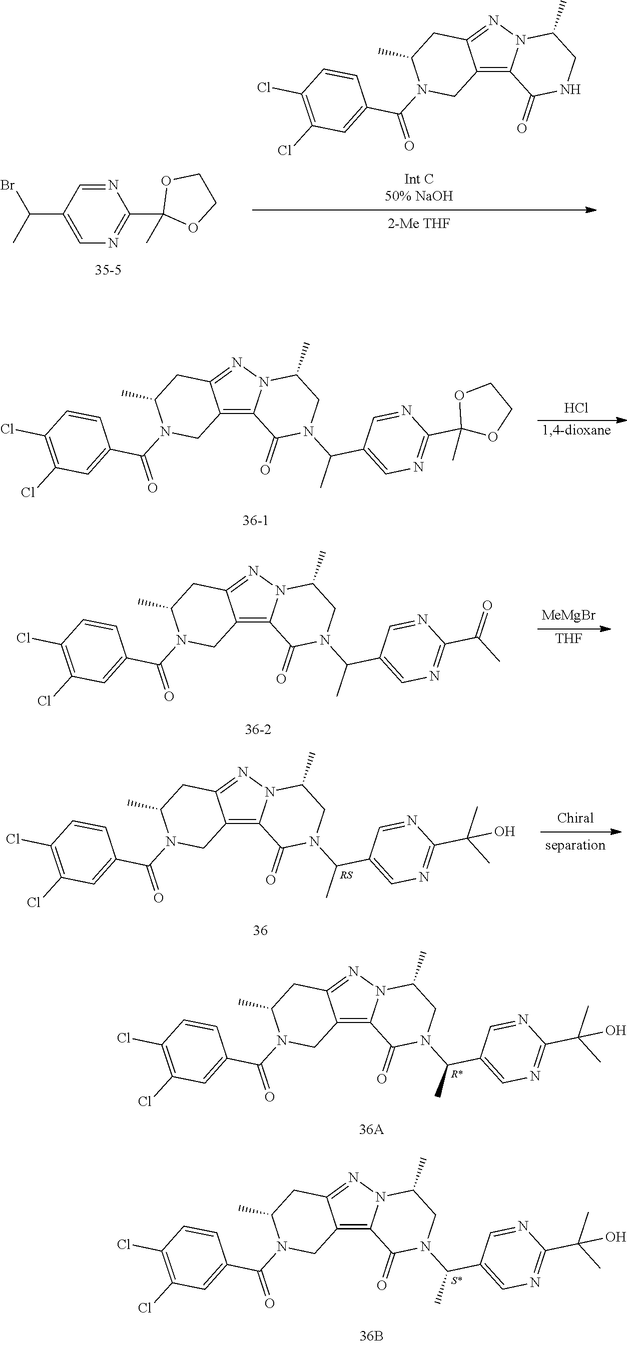

### Intermediate 36-1

### (3R,7R)-2-(3,4-Dichlorobenzoyl)-3,7-dimethyl-9-(1-(2-(2-methyl-1,3-dioxolan-2-yl)pyrimidin-5-yl)ethyl)-1,2,3,4,8,9-hexahydropyrido[4′,3′:3,4]pyrazolo[1,5-a]pyrazin-10(7H)-one

To a solution of ((3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (450 mg, 1.14 mmol, 100% purity) and 5-(1-bromoethyl)-2-(2-methyl-1,3-dioxolan-2-yl)pyrimidine 35-5 (850 mg, 98% purity, 3.05 mmol) in 2-methyltetrahydrofuran (5 mL) was added 50% wt. sodium hydroxide in water (5 mL) slowly at 0° C. After stirred at room temperature for 3 hours, the mixture was added ethyl acetate (60 mL), then washed with water (60 mL) twice, brine (120 mL), dried over Na₂SO₄₍ₛ₎ and filtered. The filtrate was concentrated and purified by C18 chromatography (acetonitrile:water (+0.02% ammonium bicarbonate)=45-65%) to give the title product (400 mg, 100% purity, 60% yield) as colorless oil. LC-MS (ESI): R_{T}=1.52 min, mass calcd. for C₂₈H₃₀Cl₂N₆O₄ 584.2, m/z found 585.2 [M+H]⁺.

### Intermediate 36-2

### (3R,7R)-9-(1-(2-Acetylpyrimidin-5-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To the solution of (3R,7R)-2-(3,4-dichlorobenzoyl)-3,7-dimethyl-9-(1-(2-(2-methyl-1,3-dioxolan-2-yl)pyrimidin-5-yl)ethyl)-1,2,3,4,8,9-hexahydropyrido[4′,3′:3,4]pyrazolo[1,5-a]pyrazin-10(7H)-one 36-1 (450 mg, 100% purity, 0.769 mmol) in 1,4-dioxane (15 mL) was added hydrogen chloride (5 mL) at 0° C. After stirred at 30° C. for 24 hours, the mixture was concentrated and adjusted to pH˜8 with sodium bicarbonate aqueous solution, extracted with ethyl acetate (60 mL) twice. The combined organic layers were washed with brine (120 mL) and dried over Na₂SO₄₍ₛ₎ and filtered. The filtrate was concentrated to give the title compound (380 mg, 91% yield, 100% purity) as yellow oil. LC-MS (ESI): R_{T}=1.55 min, mass calcd. for C₂₆H₂₆Cl₂N₆O₃ 540.1, m/z found 541.1 [M+H]⁺.

### Compound 36

### (3R,7R)-2-(3,4-Dichlorobenzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one

To the solution of (3R,7R)-9-(1-(2-acetylpyrimidin-5-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 36-2 (280 mg, 0.517 mmol, purity 100%) in tetrahydrofuran (7 mL) was added 1 M methylmagnesium bromide in tetrahydrofuran (4.1 mL, 4.1 mmol) at −78° C. After stirred at −20° C. for 3 hours, the mixture was quenched with saturated ammonium chloride solution (20 mL), extracted with dichloromethane (40 mL) for three times, the organic layers were washed with brine (80 mL), dried over Na₂SO₄₍ₛ₎ and filtered. The filtrate was concentrated under reduced pressure and purified by C18 chromatography (acetonitrile:water (+0.02% ammonium bicarbonate)=45-65%) to give the title product (190 mg, 100% purity, 65.9% yield) as white solids. LC-MS (ESI): R_{T}=1.57 min, mass calcd. for C₂₇H₃₀Cl₂N₆O₃ 556.2, m/z found 557.1 [M+H]⁺.

### Compounds 36A and 36B

### (3R,7R)-2-(3,4-Dichlorobenzoyl)-9-((R*)-1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one (36A) and (3R,7R)-2-(3,4-Dichlorobenzoyl)-9-((S*)-1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one (36B)

The racemate of (3R,7R)-2-(3,4-dichlorobenzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one 36 (80 mg, 100% purity, 0.144 mmol) was separated by chiral Prep. HPLC (separation condition: Column: Chiralpak IC 5 μm 20*250 mm; Mobile Phase: ACN:IPA:DEA=90:10:0.2 at 15 mL/min; Temp: 30° C.; Wavelength: 214 nm) to afford the title compounds 36A (30 mg, 99.6% purity, 37% yield, 100% stereopure) and 36B (30 mg, 99.6% purity, 37% yield, 99.3% stereopure) as white solids.

### 36A:

LC-MS (ESI): R_{T}=3.602 min, mass calcd. for C₂₇H₃₀Cl₂N₆O₃ 556.2, m/z found 557.3 [M+H]⁺. Chiral analysis: (Column: Chiralpak IC 5 m 4.6*250 mm; Mobile Phase: ACN:IPA:DEA=90:10:0.2 at 1 mL/min; Temp: 30° C.; Wavelength: 254 nm, R_{T}=7.300 min). ¹H NMR (400 MHz, CDCl₃) δ 8.80-8.71 (m, 2H), 7.54-7.50 (m, 2H), 7.27-7.26 (m, 1H), 6.11-4.78 (m, 2H), 4.54-4.22 (m, 4H), 3.76-3.62 (m, 1H), 3.14-2.92 (m, 2H), 2.72-2.63 (m, 1H), 1.67-1.66 (m, 3H), 1.63-1.57 (m, 3H), 1.38-1.26 (m, 9H).

### 36B:

LC-MS (ESI): R_{T}=3.026 min, mass calcd. for C₂₇H₃₀Cl₂N₆O₃ 556.2, m/z found 557.3 [M+H]⁺. Chiral analysis: (Column: Chiralpak IC 5 m 4.6*250 mm; Mobile Phase: ACN:IPA:DEA=90:10:0.2 at 1 mL/min; Temp: 30° C.; Wavelength: 254 nm, R_{T}=11.521 min). ¹H NMR (400 MHz, CDCl₃) δ 8.76-8.71 (m, 2H), 7.54-7.50 (m, 2H), 7.29-7.26 (m, 1H), 6.10-5.39 (m, 2H), 4.86-4.35 (m, 4H), 3.41-2.64 (m, 4H), 1.68-1.66 (m, 3H), 1.59-1.57 (m, 9H), 1.29-1.22 (m, 3H).

### Compounds 37A and 37B

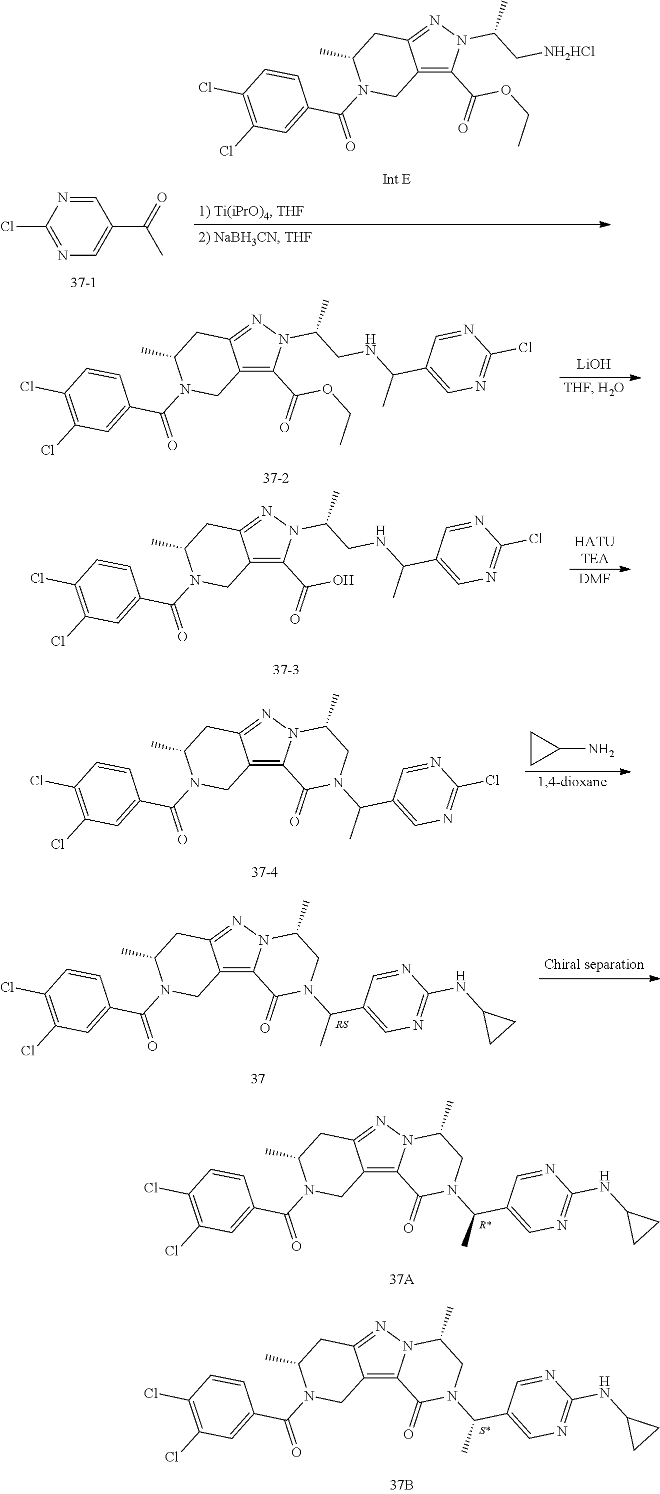

### Intermediate 37-2

### Ethyl (6R)-2-((2R)-1-((1-(2-chloropyrimidin-5-yl)ethyl)amino)propan-2-yl)-5-(3,4-dichlorobenzoyl)-6-methyl-4,5,6,7-tetrahydro-2H-pyrazolo[4,3-c]pyridine-3-carboxylate

To a solution of ethyl (R)-2-((R)-1-aminopropan-2-yl)-5-(3,4-dichlorobenzoyl)-6-methyl-4,5,6,7-tetrahydro-2H-pyrazolo[4,3-c]pyridine-3-carboxylate hydrochloride Int E (1.0 g, 95% purity, 2.00 mmol) in tetrahydrofuran (10 ml) were added 1-(2-chloropyrimidin-5-yl)ethan-1-one 37-1 (300 mg, 1.92 mmol) and tetraisopropoxytitanium (1.7 ml, 5.74 mmol). The reaction mixture was stirred at room temperature overnight. Sodium cyanoborohydride (241 mg, 3.84 mmol) was added into the reaction solution. After addition, the mixture was stirred at room temperature overnight. The reaction was quenched with water (30 ml) and filtered. The filtrate was extracted with ethyl acetate (30 ml) twice. The combined organic layers were washed with brine (20 ml), dried over Na₂SO₄₍ₛ₎ and filtered. The filtrate was concentrated under reduced pressure to give the residue, which was purified by silica gel column chromatography (petroleum ether:ethyl acetate=1:3) to give the title compound (460 mg, 99% purity by LCMS, 41% yield) as white solids. LC-MS (ESI): R_{T}=1.551 min, mass calcd. for C₂₆H₂₉Cl₃N₆O₃ 578.1 m/z found 579.1[M+H]⁺.

### Intermediate 37-3

### (6R)-2-((2R)-1-((1-(2-Chloropyrimidin-5-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid

To a solution of ethyl (6R)-2-((2R)-1-((1-(2-chloropyrimidin-5-yl)ethyl)amino)propan-2-yl)-5-(3,4-dichlorobenzoyl)-6-methyl-4,5,6,7-tetrahydro-2H-pyrazolo[4,3-c]pyridine-3-carboxylate 37-2 (610 mg, 98% purity, 1.03 mmol) in tetrahydrofuran (6 ml) was added lithium hydroxide monohydrate (86 mg, 2.05 mmol) in water (2 ml). After stirred at room temperature for 5 hours, the mixture was diluted by water (20 ml), acidified with 1 M hydrochloride aqueous solution (about 4 mL) to pH 3-4, extracted with ethyl acetate (20 mL) three times. The combined organic layers were washed with brine (10 ml), dried over Na₂SO₄₍ₛ₎ and filtered. The filtrate was concentrated to give the title compound (610 mg, 89% purity from LCMS, 95% yield) as white solids. LC-MS (ESI): R_{T}=1.30 min, mass calcd. for C₂₄H₂₅Cl₃N₆O₃ 550.1 m/z found 550.9[M+H]⁺.

### Intermediate 37-4

### (3R,7R)-9-(1-(2-Chloropyrimidin-5-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of (6R)-2-((2R)-1-((1-(2-chloropyrimidin-5-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid 37-3 (510 mg, 89% purity, 0.82 mmol) in N,N-dimethylformamide (10 ml) were added triethylamine (0.4 ml, 2.88 mmol) and 2-(3H-[1,2,3]triazolo[4,5-b]pyridin-3-yl)-1,1,3,3-tetramethyluronium hexafluorophosphate (V) (625 mg, 1.64 mmol). The mixture was stirred at room temperature for 1 hour, then water (10 mL) was added to the reaction mixture, which was extracted by ethyl acetate (20 ml) for three times. The combined organic layers were washed by brine (20 ml), dried over Na₂SO₄₍ₛ₎ and concentrated in vacuo. The residue was purified by C18 column chromatography (acetonitrile:water (0.1% ammonium bicarbonate)=0 to 73%) to give the title compound (340 mg, 100% purity from LCMS, 77% yield). LC-MS (ESI): R_{T}=1.360 min, mass calcd. for C₂₄H₂₃Cl₃N₆O₂ 532.1 m/z found 533.1[M+H]⁺.

### Compound 37

### (3R,7R)-9-(1-(2-(Cyclopropylamino)pyrimidin-5-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of cyclopropylamine (3 ml) in 1,4-dioxane (3 ml) was added (3R,7R)-9-(1-(2-chloropyrimidin-5-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 37-4 (250 mg, 100% purity, 0.47 mmol). The reaction was heated to 60° C. and stirred overnight. The mixture was diluted with water (20 ml), then was extracted with dichloromethane (20 ml) for three times. The combined organic layers were washed with brine (20 ml), dried over Na₂SO₄₍ₛ₎, filtered and concentrated to give the crude product, which was purified by silica gel column chromatography (dichloromethane:methanol=1:0 to 40:1) to give the title compound (220 mg, 99% purity from LCMS, 84% yield) as white solids. LC-MS (ESI): R_{T}=1.370 min, mass calcd. for C₂₇H₂₉Cl₂N₇O₂ 553.2 m/z found 554.1[M+H]⁺.

### Compounds 37A and 37B

### (3R,7R)-9-((R*)-1-(2-(cyclopropylamino)pyrimidin-5-yl)ethyl)-2-(3,4-dichloro benzoyl)-3,7-dimethyl-1,2,3,4,8,9-hexahydropyrido[4′,3′:3,4]pyrazolo[1,5-a]pyrazin-10(7H)-one (37A) and (3R,7R)-9-((S*)-1-(2-(cyclopropylamino)pyrimidin-5-yl)ethyl)-2-(3,4-dichloro benzoyl)-3,7-dimethyl-1,2,3,4,8,9-hexahydropyrido[4′,3′:3,4]pyrazolo[1,5-a]pyrazin-10(7H)-one (37B)

A racemic mixture of (3R,7R)-9-(1-(2-(cyclopropylamino)pyrimidin-5-yl)ethyl)-2-(3,4-dichlorobenzoyl)-3,7-dimethyl-1,2,3,4,8,9-hexahydropyrido[4′,3′:3,4]pyrazolo [1,5-a]pyrazin-10(7H)-one 37 (220 mg, 99% purity, 0.39 mmol) was separated by chiral HPLC (Column: Chiralpak IB N-5, 5 μm 20*250 mm; Mobile Phase: ACN=100 at 30 g/min; Temp: 30° C.; Wavelength: 254 nm) to afford the title compounds 37A (83 mg, 38% yield, 99.5% purity from LCMS, 100% stereopure) and 37B (82 mg, 99.8% purity from LCMS, 37.6% yield, 100% stereopure) as white solids.

### 37A:

LC-MS (ESI): R_{T}=3.138 min, mass calcd. for C₂₇H₂₉Cl₂N₇O₂ 553.2 m/z found 554.2 [M+H]⁺. Chiral analysis (Chiralpak IB N-5 5 m 4.6*250 mm; Mobile Phase: ACN=100 at 1 mL/min; Temp: 30° C.; Wavelength: 254 nm; R_{T}=6.735 min). ¹H NMR (400 MHz, DMSO-d₆) δ 8.39-8.23 (m, 2H), 7.80-7.69 (m, 2H), 7.48-7.39 (m, 2H), 5.75-5.15 (m, 2H), 4.58-4.35 (m, 2H), 4.22-4.04 (m, 1H), 3.84-3.69 (m, 1H), 3.21-3.07 (m, 1H), 2.97-2.86 (m, 1H), 2.67-2.54 (m, 2H), 1.60-1.42 (m, 3H), 1.29 (d, J=6.4 Hz, 3H), 1.23-1.06 (m, 3H), 0.67-0.61 (m, 2H), 0.49-0.40 (m, 2H).

### 37B:

LC-MS (ESI): R_{T}=3.159 min, mass calcd. for C₂₇H₂₉Cl₂N₇O₂ 553.2 m/z found 554.2 [M+H]⁺. Chiral analysis (Chiralpak IB N-5 5 m 4.6*250 mm; Mobile Phase: ACN=100 at 1 mL/min; Temp: 30° C.; Wavelength: 254 nm; R_{T}=11.381 min). ¹H NMR (400 MHz, DMSO-d₆) δ 8.37-8.23 (m, 2H), 7.78-7.71 (m, 2H), 7.49-7.40 (m, 2H), 5.77-5.18 (m, 2H), 4.61-4.32 (m, 2H), 4.22-4.07 (m, 1H), 3.53-3.43 (m, 2H), 2.98-2.88 (m, 1H), 2.67-2.55 (m, 2H), 1.56-1.38 (m, 6H), 1.24-1.08 (m, 3H), 0.67-0.62 (m, 2H), 0.46-0.43 (m, 2H).

### Compounds 38A and 38B

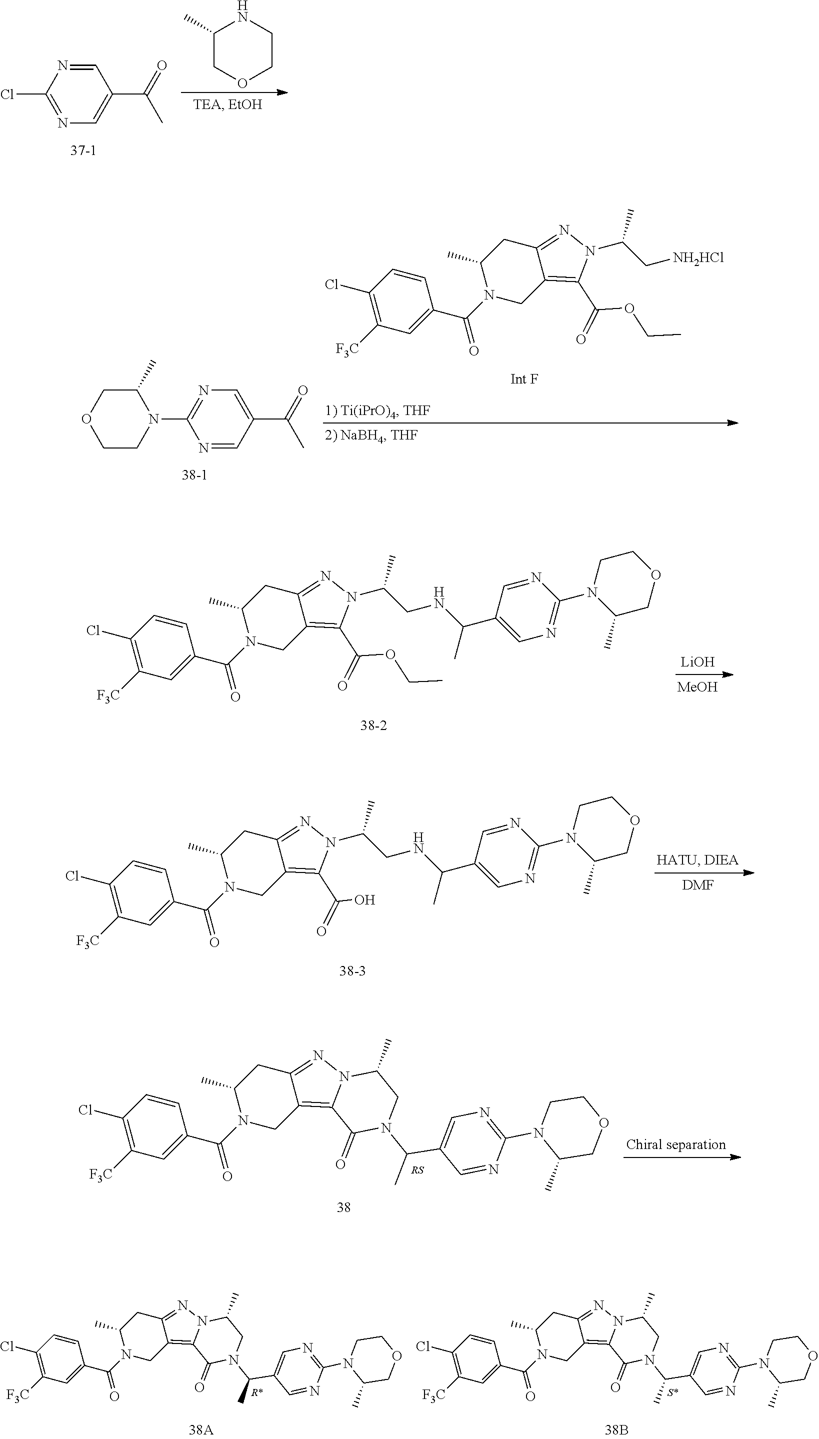

### Intermediate 38-1

### (S)-1-(2-(3-Methylmorpholino)pyrimidin-5-yl)ethan-1-one

To a solution of 1-(2-chloropyrimidin-5-yl)ethan-1-one 37-1 (2.0 g, 12.8 mmol) in ethanol (20 mL) were added (R)-3-methylmorpholine (1.6 g, 15.8 mmol) and triethylamine (3.7 mL, 25.6 mmol). After stirred at 75° C. for 16 hours, the reaction mixture was cooled down to room temperature and filtered. The filtrate was concentrated under reduced pressure to give the crude, which was purified by C18 column (acetonitrile:water=55% to 75%) to give the title compound (2.1 g, 90% purity, 66.9% yield) as yellow solids. ¹H NMR (400 MHz, CDCl₃) δ 8.53 (s, 2H), 4.89-4.83 (m, 1H), 4.55-4.51 (m, 1H), 4.03-3.99 (m, 1H), 3.81-3.78 (m, 1H), 3.71-3.68 (m, 1H), 3.58-3.51 (m, 1H), 3.38-3.31 (m, 1H), 2.48 (s, 3H), 1.35-1.34 (m, 3H).

### Intermediate 38-2

### Ethyl (6R)-5-(4-chloro-3-(trifluoromethyl)benzoyl)-6-methyl-2-((2R)-1-((1-(2-((S)-3-ethylmorpholino)pyrimidin-5-yl)ethyl)amino)propan-2-yl)-4,5,6,7-tetrahydro-2H-pyrazolo[4,3-c]pyridine-3-carboxylate

To a solution of ethyl (R)-2-((R)-1-aminopropan-2-yl)-5-(4-chloro-3-(trifluoro methyl)benzoyl)-6-methyl-4,5,6,7-tetrahydro-2H-pyrazolo[4,3-c]pyridine-3-carboxylate hydrochloride Int F (1.9 g, 90% purity, 3.36 mmol) and (S)-1-(2-(3-methylmorpholino)pyrimidin-5-yl)ethan-1-one 38-1 (1.00 g, 90% purity, 3.49 mmol) in tetrahydrofuran (25 mL) was added titanium(IV) propan-2-olate (2 mL, 6.83 mmol). After stirred at 70° C. for 16 hours, the reaction mixture was cooled down to 0° C. and added sodium borohydride (126 mg, 3.33 mmol). The reaction was quenched with ammonium chloride aqueous (10 mL), filtered with kieselguhr. The filtrate was concentrated to give the crude, which was purified by C18 column (acetonitrile:water=40% to 60%) to give the title compound (410 mg, 76% purity, 13.7% yield) as yellow solids. LC-MS (ESI): R_{T}=1.83 min, mass calcd. for C₃₂H₃₉ClF₃N₇O₄ 677.3, m/z found 678 [M+H]⁺.

### Intermediate 38-3

### (6R)-5-(4-Chloro-3-(trifluoromethyl)benzoyl)-6-methyl-2-((2R)-1-((1-(2-((S)-3-methylmorpholino)pyrimidin-5-yl)ethyl)amino)propan-2-yl)-4,5,6,7-tetrahydro-2H-pyrazolo[4,3-c]pyridine-3-carboxylic acid

To a solution of ethyl (6R)-5-(4-chloro-3-(trifluoromethyl)benzoyl)-6-methyl-2-((2R)-1-((1-(2-((S)-3-methylmorpholino)pyrimidin-5-yl)ethyl)amino)propan-2-yl)-4,5,6,7-tetrahydro-2H-pyrazolo[4,3-c]pyridine-3-carboxylate 38-2 (410 mg, 76% purity, 0.459 mmol) in methanol (4.5 mL) was added solution of lithium hydroxide monohydrate (39 mg, 0.929 mmol) in water (1.5 mL) at 0° C. After stirred at 0° C. for 1 hour, the mixture was added 1 M hydrochloride aqueous solution (2 mL) and concentrated under reduced pressure to give the title compound (350 mg, 65% purity, 76.2% yield) as yellow solids. LC-MS (ESI): R_{T}=1.40 min, mass calcd. for C₃₀H₃₅ClF₃N₇O₄ 649.2, m/z found 650.0 [M+H]⁺.

### Compound 38

### (3R,7R)-2-(4-Chloro-3-(trifluoromethyl)benzoyl)-3,7-dimethyl-9-(1-(2-((S)-3-methylmorpholino)pyrimidin-5-yl)ethyl)-1,2,3,4,8,9-hexahydropyrido[4′,3′:3,4]pyrazolo[1,5-a]pyrazin-10(7H)-one

The mixture of (6R)-5-(4-chloro-3-(trifluoromethyl)benzoyl)-6-methyl-2-((2R)-1-((1-(2-((S)-3-methylmorpholino)pyrimidin-5-yl)ethyl)amino)propan-2-yl)-4,5,6,7-tetrahydro-2H-pyrazolo[4,3-c]pyridine-3-carboxylic acid 38-3 (350 mg, 65% purity, 0.350 mmol), N-ethyl-N-isopropylpropan-2-amine (255 mg, 1.97 mmol), 2-(3H-[1,2,3]triazolo[4,5-b]pyridin-3-yl)-1,1,3,3-tetramethylisouronium hexafluorophosphate (V) (267 mg, 0.702 mmol) in N,N-dimethylformamide (10 mL) was stirred at room temperature for 14 hours, then was acidified to pH=6 with 1 M hydrochloride aqueous solution and extracted with ethyl acetate (30 mL) twice. The combined organic layers were washed with water (30 mL) and brine (35 mL), dried over Na₂SO₄₍ₛ₎ and filtered. The filtrate was concentrated under reduced pressure to give the crude, which was purified by C18 column (acetonitrile:water=40% to 60%) to give the title compound (200 mg, 90% purity, 81.4% yield) as white solids. ¹H NMR (400 MHz, CDCl₃) δ 8.33-7.29 (m, 2H), 7.80-7.79 (m, 1H), 7.59-7.52 (m, 2H), 5.95-5.43 (m, 2H), 4.81-4.67 (m, 2H), 4.53-4.29 (m, 3H), 4.00-3.96 (m, 1H), 3.79-3.76 (m, 1H), 3.70-3.51 (m, 3H), 3.34-3.23 (m, 2H), 3.10-3.03 (m, 1H), 2.71-2.65 (m, 1H), 1.54-1.50 (m, 3H), 1.43-1.37 (m, 3H), 1.30-1.27 (m, 6H).

### Compounds 38A and 38B

### (3R,7R)-2-(4-chloro-3-(trifluoromethyl)benzoyl)-3,7-dimethyl-9-((R*)-1-(2-((S)-3-methylmorpholino)pyrimidin-5-yl)ethyl)-1,2,3,4,8,9-hexahydropyrido[4′,3′:3,4]pyrazolo[1,5-a]pyrazin-10(7H)-one (38A) and (3R,7R)-2-(4-chloro-3-(trifluoromethyl)benzoyl)-3,7-dimethyl-9-((S*)-1-(2-((S)-3-methylmorpholino)pyrimidin-5-yl)ethyl)-1,2,3,4,8,9-hexahydropyrido[4′,3′:3,4]pyrazolo[1,5-a]pyrazin-10(7H)-one (38B)

The racemate of (3R,7R)-2-(4-chloro-3-(trifluoromethyl)benzoyl)-3,7-dimethyl-9-(1-(2-((S)-3-methylmorpholino)pyrimidin-5-yl)ethyl)-1,2,3,4,8,9-hexahydropyrido[4′,3′:3,4]pyrazolo[1,5-a]pyrazin-10(7H)-one 38 (200 mg, 90% purity, 0.285 mmol) was separated by chiral Prep. HPLC separation condition: (Column: Chiralpak ID 5 m 20*250 mm; Mobile Phase: ACN:IPA=90:10 at 30 mL/min; Temp: 30° C.; Wavelength: 254 nm) to give the title compounds 38A (64.8 mg, 100% purity, 36.0% yield, 100% stereopure) as white solids and 38B (39.2 mg, 99.7% purity, 21.7% yield, 99.9% stereopure) as white solids.

### 38A:

LC-MS (ESI): R_{T}=3.836 min, mass calcd. for C₃₀H₃₃ClF₃N₇O₃ 631.2, m/z found 632.3 [M+H]⁺. Chiral analysis (Column: Chiralpak ID 5 m 4.6*250 mm; Mobile Phase: ACN:IPA=90:10 at 30 mL/min; Temp: 30° C.; Wavelength: 254 nm; R_{T}=5.166 min). ¹H NMR (400 MHz, CDCl₃) δ 8.33 (m, 2H), 7.79 (s, 1H), 7.59-7.52 (m, 2H), 5.94-5.45 (m, 2H), 4.81-4.30 (m, 5H), 4.00-3.96 (m, 1H), 3.79-3.50 (m, 4H), 3.31-3.23 (m, 1H), 3.15-2.92 (m, 2H), 2.70-2.66 (m, 1H), 1.55-1.54 (m, 3H), 1.38-1.37 (m, 3H), 1.29-1.27 (m, 6H). ¹⁹F NMR (376 MHz, CDCl₃) δ −62.8.

### 38B:

LC-MS (ESI): R_{T}=3.843 min, mass calcd. for C₃₀H₃₃ClF₃N₇O₃ 631.2, m/z found 632.3 [M+H]⁺. Chiral analysis (Column: Chiralpak ID 5 m 4.6*250 mm; Mobile Phase: ACN:IPA=90:10 at 30 mL/min; Temp: 30° C.; Wavelength: 254 nm; R_{T}=7.063 min). ¹H NMR (400 MHz, CDCl₃) δ 8.29 (m, 2H), 7.80 (s, 1H), 7.59-7.52 (m, 2H), 5.92-5.46 (m, 2H), 4.83-4.30 (m, 5H), 4.00-3.97 (m, 1H), 3.79-3.66 (m, 2H), 3.56-3.50 (m, 1H), 3.33-3.05 (m, 4H), 2.71-2.66 (m, 1H), 1.57-1.55 (m, 6H), 1.30-1.28 (m, 6H). ¹⁹F NMR (376 MHz, CDCl₃) −62.8.

### Compounds 39A and 39B

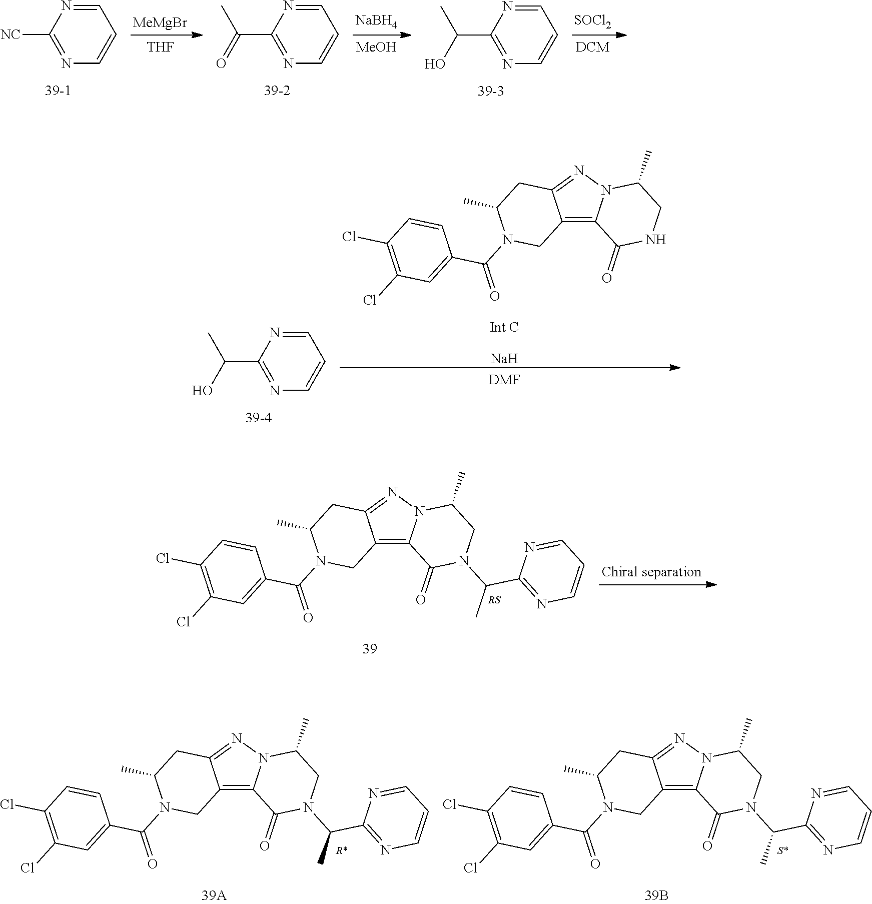

### Intermediate 39-2

### 1-(Pyrimidin-2-yl)ethan-1-one

A solution of 2-cyano-pyrimidine 39-1 (5.0 g, 47.6 mmol) in tetrahydrofuran (40 mL) was cooled to −20° C. and treated with 1 M solution of methyl magnesium bromide in tetrahydrofuran (57 mL, 57 mmol). After stirred at 0° C. for 20 min, the reaction mixture was poured into 2 M hydrochloride aqueous solution (200 mL) and the solution was warmed to 20° C. and stirred for 40 minutes, then adjusted pH to 7-8 by addition of saturated sodium bicarbonate solution. The aqueous phase was extracted with dichloromethane (100 mL) for three times. The combined organic layers were concentrated under reduced pressure and the crude compound was purified by column chromatography (petroleum ether:ethyl acetate from 2:1 to 1:1) to give the title compound (3.0 g, 90% purity ¹H NMR, 47% yield) as yellow solids. ¹H NMR (400 MHz, CDCl₃) δ 8.96 (d, J=4.8 Hz, 2H), 7.53-7.50 (m, 1H), 2.80 (s, 3H)

### Intermediate 39-3

### 1-(Pyrimidin-2-yl)ethan-1-ol

To a mixture of 1-(pyrimidin-2-yl)ethan-1-one 39-2 (750 mg, 90% purity, 5.53 mmol) in methanol (10 mL) was added sodium borohydride (105 mg, 2.78 mmol). After stirred at 0° C. for 0.5 hour, the reaction mixture was quenched by saturated ammonium chloride aqueous solution (3 mL), dried over Na₂SO₄₍ₛ₎, filtered and concentrated under reduced pressure to give the title compound (700 mg, 90% purity from ¹H NMR, 92% yield) as yellow oil. ¹H NMR (400 MHz, CDCl₃) δ 8.75 (d, J=4.8 Hz, 2H), 7.23-7.22 (m, 1H), 4.96 (q, J=6.4 Hz, 1H), 1.58 (q, J=6.4 Hz, 3H).

### Intermediate 39-4

### 2-(1-Chloroethyl)pyrimidine

To a solution of 1-(pyrimidin-2-yl)ethan-1-ol 39-3 (300 mg, 90% purity, 2.18 mmol) in dichloromethane (7 mL) was added sulfurous dichloride (0.5 mL, 7.04 mmol) at room temperature. After stirred at 40° C. for 0.5 hour, the mixture was concentrated to give the title compound (340 mg, 90% purity from ¹H NMR, 99% yield) as yellow oil. ¹H NMR (400 MHz, CDCl₃) δ 8.98-8.95 (m, 2H), 7.53-7.38 (m, 1H), 5.38 (q, J=6.4 Hz, 1H), 1.91 (q, J=6.8 Hz, 3H).

### Intermediate 39

### ((3R,7R)-2-(3,4-Dichlorobenzoyl)-3,7-dimethyl-9-(1-(pyrimidin-2-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (300 mg, 90% purity, 0.687 mmol) in N,N-dimethylformamide (6 mL) was added 60% wt. sodium hydride in mineral oil (110 mg, 2.75 mmol). After stirred at 0° C. for 0.5 hour, 2-(1-chloroethyl)pyrimidine 39-4 (217 mg, 90% purity, 1.37 mmol) was added into the mixture. After stirred at 45° C. for 14 hours, the reaction mixture was quenched with water (30 mL) and extracted with ethyl acetate (25 mL) for three times. The combined organic layers were dried over Na₂SO₄₍ₛ₎ and filtered. The filtrate was concentrated and purified by C18 column (acetonitrile:water=5% to 100%) to give the title compound (200 mg, 100% purity, 58% yield) as white solids. LC-MS (ESI): R_{T}=1.50 min, mass calcd. for C₂₄H₂₄Cl₂N₆O₂ 498.1 mz found 499.1[M+H]⁺.

### Compounds 39A and 39B

### (3R,7R)-2-(3,4-dichlorobenzoyl)-3,7-dimethyl-9-((R*)-1-(pyrimidin-2-yl)ethyl)-1,2,3,4,8,9-hexahydropyrido[4′,3′:3,4]pyrazolo[1,5-a]pyrazin-10(7H)-one (39A) and (3R,7R)-2-(3,4-dichlorobenzoyl)-3,7-dimethyl-9-((S*)-1-(pyrimidin-2-yl)ethyl)-1,2,3,4,8,9-hexahydropyrido[4′,3′:3,4]pyrazolo[1,5-a]pyrazin-10(7H)-one (39B) ((3R,7R)-2-(3,4-dichlorobenzoyl)-3,7-dimethyl-9-(1-(pyrimidin-2-yl)ethyl)-1,2,3,4,8,9-hexahydropyrido[4′,3′:3,4]pyrazolo[1,5-a]pyrazin-10(7H)-one 39 (200 mg, 100% purity, 0.400 mmol) was separated by chiral Prep. HPLC (separation condition: Column: Chiralpak IB 5 m 20*250 mm; Mobile Phase: Hexane:EtOH=60:40 at 15 mL/min; Temp: 30° C.; Wavelength: 254 nm) to give the title compounds 39A (42.7 mg, 99.8% purity, 21% yield, 100% stereopure) as white solids and 39B (39.1 mg, 99.7% purity, 19% yield, 99.3% stereopure) as white solids

### 39A:

LC-MS (ESI): R_{T}=2.159 min, mass calcd. for C₂₄H₂₄Cl₂N₆O₂ 498.1 mz found 499.2[M+H]⁺. Chiral analysis (Column: Superchiral IB 5 m 4.6*250 mm; Mobile Phase: Hexane:EtOH=60:40 at 1 mL/min; Temp: 30° C.; Wavelength: 254 nm; Rt=9.394 min). ¹H NMR (400 MHz, CDCl₃) δ 8.70 (d, J=4.4 Hz, 2H), 7.52-7.47 (m, 2H), 7.25-7.19 (m, 2H), 6.17-6.04 (m, 1H), 5.73-5.41 (m, 1H), 4.85-4.30 (m, 3H), 3.88-3.77 (m, 1H), 3.51-3.46 (m, 1H), 3.14-2.89 (m, 1H), 2.76-2.65 (m, 1H), 1.68 (d, J=6.8 Hz, 3H), 1.58 (d, J=6.4 Hz, 3H), 1.31-1.27 (m, 3H).

### 39B:

LC-MS (ESI): R_{T}=2.123 min, mass calcd. for C₂₄H₂₄Cl₂N₆O₂ 498.1 mz found 499.2[M+H]⁺. Chiral analysis (Column: Superchiral IB 5 m 4.6*250 mm; Mobile Phase: Hexane:EtOH=60:40 at 1 mL/min; Temp: 30° C.; Wavelength: 254 nm; Rt=10.654 min). ¹H NMR (400 MHz, CDCl₃) δ 8.73 (d, J=4.8 Hz, 2H), 7.52-7.47 (m, 2H), 7.26-7.20 (m, 2H), 6.14-6.06 (m, 1H), 5.78-5.39 (m, 1H), 4.88-4.31 (m, 3H), 3.80-3.72 (m, 1H), 3.60-3.59 (m, 1H), 3.12-2.88 (m, 1H), 2.73-2.63 (m, 1H), 1.67 (d, J=6.0 Hz, 3H), 1.45 (d, J=6.4 Hz, 3H), 1.31-1.19 (m, 3H).

### Compounds 40A and 40B

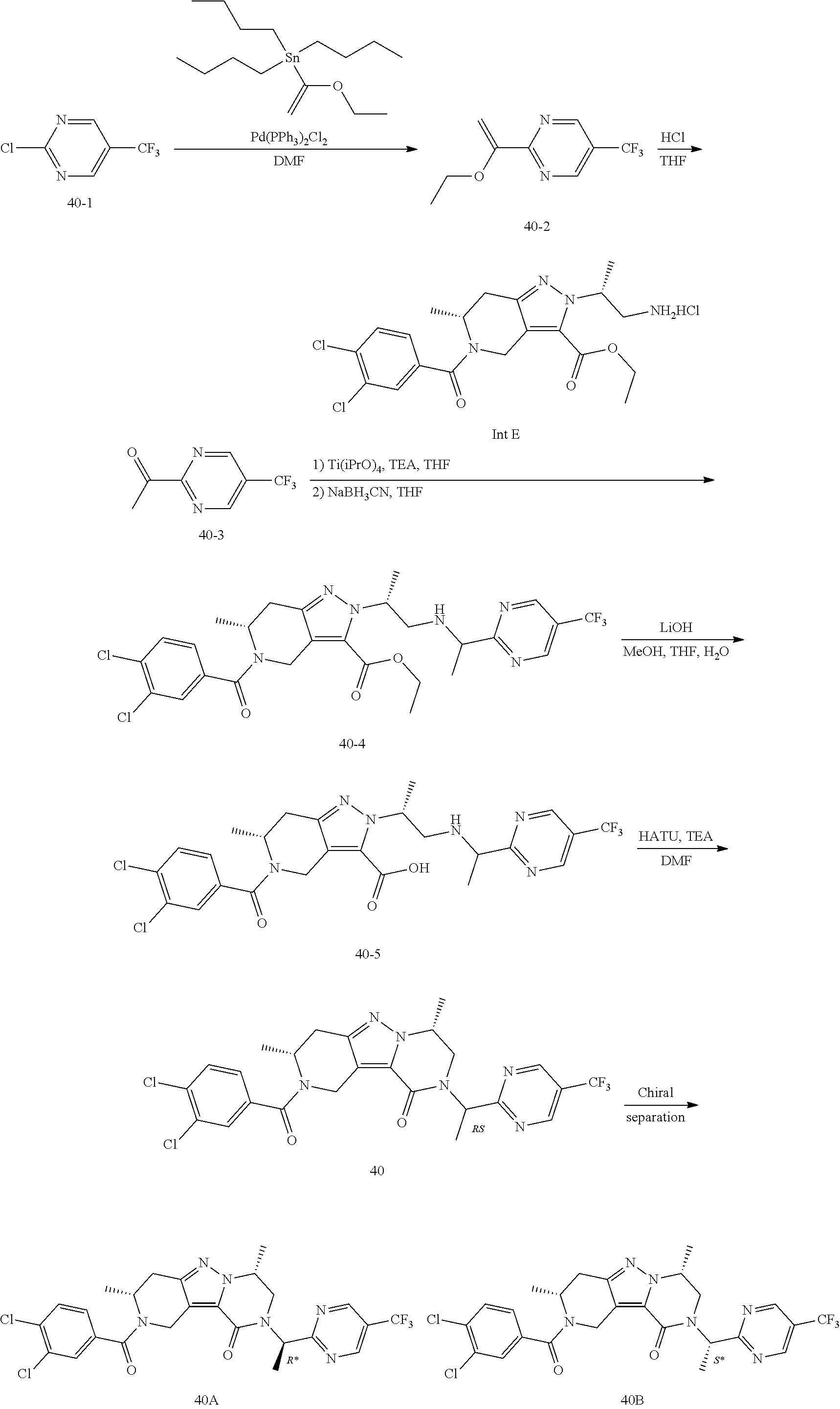

### Intermediate 40-2

### 2-(1-Ethoxyvinyl)-5-(trifluoromethyl)pyrimidine

To a mixture of 2-chloro-5-(trifluoromethyl)pyrimidine 40-1 (3.5 g, 100% purity, 19.2 mmol) and tributyl(1-ethoxyvinyl)stannane (8.3 g, 23.0 mmol) in N,N-dimethylformamide (35 mL) was added bis(triphenylphosphoranyl)palladium(IV) chloride (1.0 g, 1.42 mmol). After stirred at 100° C. for 3 hours under nitrogen atmosphere, the solution was cooled down to room temperature. The mixture was added water (20 mL) and potassium fluoride (7.0 g). After stirred at room temperature for 1 hour, the resulting reaction mixture was filtrated. The filtrate was extracted with ethyl acetate (200 mL). The organic phase was concentrated and purified by silica gel column chromatography (petroleum ether:ethyl acetate=100:1 to 10:1) to give the title product (5.4 g, 67% purity from LCMS, 86% yield) as red oil. LC-MS (ESI): R_{T}=1.57 min, mass calcd. for C₉H₉F₃N₂O 218.1, m/z found 219.0 [M+H]⁺.

### Intermediate 40-3

### 1-(5-(Trifluoromethyl)pyrimidin-2-yl)ethan-1-one

A solution of 2-(1-ethoxyvinyl)-5-(trifluoromethyl)pyrimidine 40-2 (5.1 g, 67% purity, 15.7 mmol) and 2 M hydrogen chloride in water (20 mL) and tetrahydrofuran (20 mL) was stirred at 40° C. for 3 hours. The solution was added water (50 mL) and extracted with dichloromethane (40×2 mL). The combined organic layers were concentrated under reduced pressure and purified by C18 column (acetonitrile:water=5% to 75%) to give the title compound (2.4 g, 95% purity from ¹H NMR, 77% yield) as brown solids. LC-MS (ESI): R_{T}=1.25 min, mass calcd. for C₇H₅F₃N₂O 190.0, m/z found 191.0[M+H]⁺. ¹H NMR (400 MHz, DMSO-d₆) δ 9.49 (s, 2H), 2.72 (s, 3H).

### Intermediate 40-4

### (6R)-Ethyl 5-(3,4-dichlorobenzoyl)-6-methyl-2-((2R)-1-((1-(5-(trifluoromethyl) pyrimidin-2-yl)ethyl)amino)propan-2-yl)-4,5,6,7-tetrahydro-2H-pyrazolo[4,3-c]pyridine-3-carboxylate

To a mixture of (R)-ethyl 2-((R)-1-aminopropan-2-yl)-5-(3,4-dichlorobenzoyl)-6-methyl-4,5,6,7-tetrahydro-2H-pyrazolo[4,3-c]pyridine-3-carboxylate hydrochloride Int E (522 mg, 88.7% purity, 0.973 mmol) and 1-(5-(trifluoromethyl)pyrimidin-2-yl)ethanone 40-3 (250 mg, 95% purity, 1.32 mmol) in dry tetrahydrofuran (15 mL) were added tetraisopropoxytitanium (610 mg, 2.15 mmol) and triethylamine (147 mg, 1.45 mmol). After stirred at room temperature for 3 hours, the mixture was added sodium cyanotrihydroborate (135 mg, 2.15 mmol) at 0° C. The mixture was stirred at room temperature for 2 hours and then poured into water (50 mL), extracted with ethyl acetate (120 mL). The combined organic layers were concentrated under reduced pressure and purified by silica gel column chromatography (dichloromethane:methanol=100:1 to 10:1) to give the title compound (600 mg, 80% purity from LCMS, 80% yield) as white solids. LC-MS (ESI): R_{T}=1.50 min, mass calcd. for C₂₇H₂₉Cl₂F₃N₆O₃ 612.2, m/z found 613.2 [M+H]⁺.

### Intermediate 40-5

### Lithium (6R)-5-(3,4-dichlorobenzoyl)-6-methyl-2-((2R)-1-((1-(5-(trifluoromethyl) pyrimidin-2-yl)ethyl)amino)propan-2-yl)-4,5,6,7-tetrahydro-2H-pyrazolo[4,3-c]pyridine-3-carboxylate

To a mixture of (6R)-ethyl 5-(3,4-dichlorobenzoyl)-6-methyl-2-((2R)-1-((1-(5-(trifluoromethyl)pyrimidin-2-yl)ethyl)amino)propan-2-yl)-4,5,6,7-tetrahydro-2H-pyrazolo[4,3-c]pyridine-3-carboxylate 40-4 (600 mg, 80% purity, 0.782 mmol) in tetrahydrofuran (4 mL), methanol (4 mL) and water (2 mL) was added lithium hydroxide hydrate (103 mg, 2.46 mmol) at 0° C. After stirred at room temperature for 1 hour, the mixture was concentrated under reduced pressure to give the title compound (500 mg, 85% purity from LCMS, 92% yield) as white solids. LC-MS (ESI): R_{T}=1.40 min, mass calcd. for C₂₅H₂₄Cl₂F₃LiN₆O₃ 584.1, m/z found 585.1 [M+H]⁺.

### Compound 40

### (3R,7R)-2-(3,4-Dichlorobenzoyl)-3,7-dimethyl-9-(1-(5-(trifluoromethyl)pyrimidin-2-yl)ethyl)-1,2,3,4,8,9-hexahydropyrido[4′,3′:3,4]pyrazolo[1,5-a]pyrazin-10(7H)-one

To a mixture of lithium (6R)-5-(3,4-dichlorobenzoyl)-6-methyl-2-((2R)-1-((1-(5-(trifluoromethyl)pyrimidin-2-yl)ethyl)amino)propan-2-yl)-4,5,6,7-tetrahydro-2H-pyrazolo[4,3-c]pyridine-3-carboxylate 40-5 (500 mg, 85% purity, 0.719 mmol) in N,N-dimethylformamide (10 mL) were added 2-(7-azabenzotriazol-1-yl)-N,N,N′,N′-tetramethyluronium hexafluorophosphate (330 mg, 0.868 mmol) and triethylamine (180 mg, 1.78 mmol) at 0° C. After stirred at room temperature for 30 minutes, the reaction was purified by C18 column (acetonitrile:water=05% to 80%) to give the title compound (290 mg, 100% purity from LCMS, 71% yield) as white solids. LC-MS (ESI): R_{T}=1.40 min, mass calcd. for C₂₅H₂₃Cl₂F₃N₆O₂ 566.1, m/z found 567.1 [M+H]⁺.

### Compounds 40A and 40B

### (3R,7R)-2-(3,4-dichlorobenzoyl)-3,7-dimethyl-9-((R*)-1-(5-(trifluoromethyl)pyrimidin-2-yl)ethyl)-1,2,3,4,8,9-hexahydropyrido[4′,3′:3,4]pyrazolo[1,5-a]pyrazin-10(7H)-one (40A) and (3R,7R)-2-(3,4-dichlorobenzoyl)-3,7-dimethyl-9-((S*)-1-(5-(trifluoromethyl)pyrimidin-2-yl)ethyl)-1,2,3,4,8,9-hexahydropyrido[4′,3′:3,4]pyrazolo[1,5-a]pyrazin-10(7H)-one (40B)

The racemate of (3R,7R)-2-(3,4-dichlorobenzoyl)-3,7-dimethyl-9-(1-(5-(trifluoro methyl)pyrimidin-2-yl)ethyl)-1,2,3,4,8,9-hexahydropyrido[4′,3′:3,4]pyrazolo[1,5-a]pyrazin-10(7H)-one 40 (290 mg, 100% purity, 0.325 mmol) was separated by chiral Prep.HPLC (separation method: Column: Chiralpak IC, 5 m 30*250 mm; Mobile Phase: Hex:ethanol=30:70 at 60 mL/min; Col. Temp: 30° C.; Wavelength: 214 nm) to give the title products 40A (71.3 mg, 99.1% purity, 24% yield, 100% stereopure) as white solids and 40B (118.6 mg, 99.4% purity, 99.9% ee, 41% yield, 100% stereopure) as white solids.

### 40A:

LC-MS (ESI): R_{T}=4.296 min, mass calcd. for C₂₅H₂₃Cl₂F₃N₆O₂ 566.1, m/z found 567.2 [M+H]⁺. Chiral analysis (Column: Chiralpak IC, 5 m 4.6*250 mm; Mobile Phase: Hex:ethanol=30:70 at 1 mL/min; Col. Temp: 30° C.; Wavelength: 254 nm, Back pressure: 100 bar; R_{T}=8.598 min). ¹H NMR (400 MHz, CDCl₃) δ 8.94 (s, 2H), 7.51-7.43 (m, 2H), 7.23 (d, J=8.4 Hz, 1H), 6.19-6.00 (m, 1H), 5.80-5.32 (m, 1H), 4.89-4.20 (m, 3H), 4.02-3.79 (m, 1H), 3.56-3.51 (m, 1H), 3.21-2.89 (m, 1H), 2.76-2.58 (m, 1H), 1.72 (d, J=6.8 Hz, 3H), 1.61 (d, J=6.4 Hz, 3H), 1.33-1.19 (m, 3H). ¹⁹F NMR (376 MHz, CDCl₃) −62.37.

### 40B:

LC-MS (ESI): R_{T}=4.299 min, mass calcd. for C₂₅H₂₃Cl₂F₃N₆O₂ 566.1, m/z found 567.2 [M+H]⁺. Chiral analysis (Column: Chiralpak IC, 5 m 4.6*250 mm; Mobile Phase: Hex:ethanol=30:70 at 1 mL/min; Col. Temp: 30° C.; Wavelength: 254 nm, R_{T}=13.030 min). ¹H NMR (400 MHz, CDCl₃) δ 8.96 (s, 2H), 7.53-7.44 (m, 2H), 7.23 (d, J=7.6 Hz, 1H), 6.16-5.96 (m, 1H), 5.76-5.31 (m, 1H), 4.87-4.20 (m, 3H), 3.89-3.73 (m, 1H), 3.67-3.62 (m, 1H), 3.17-2.91 (m, 1H), 2.75-2.60 (m, 1H), 1.77-1.66 (m, 3H), 1.55 (d, J=6.4 Hz, 3H), 1.31-1.18 (m, 3H). ¹⁹F NMR (376 MHz, CDCl₃) −62.37.

### Compounds 41A and 41B

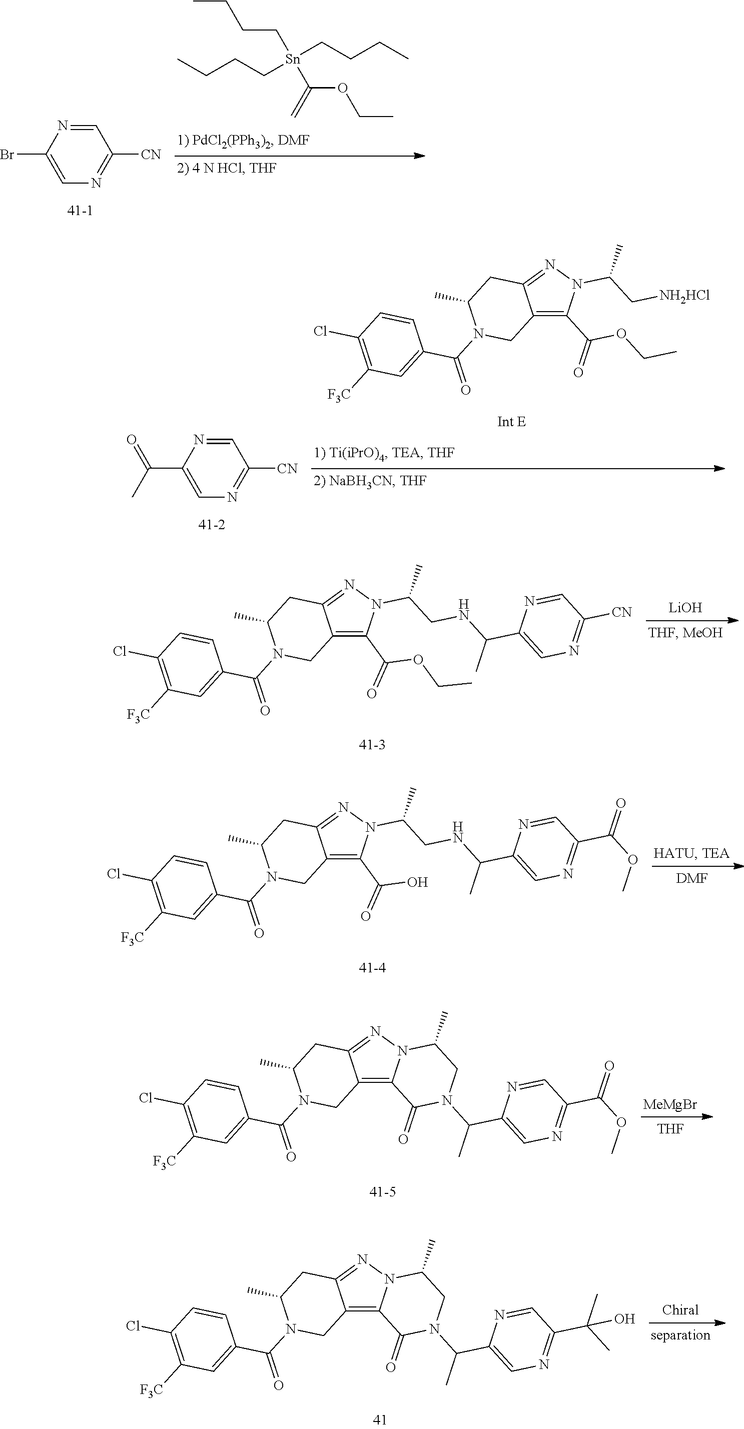

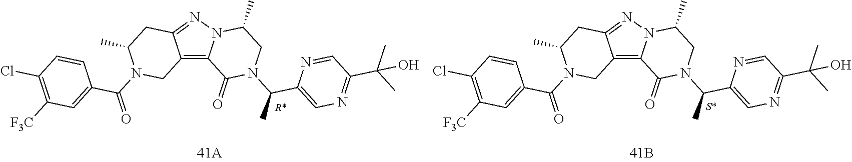 [SMILES: C[C@@H]1Cc2nn3c(c2CN1C(=O)c1ccc(Cl)c(C(F)(F)F)c1)C(=O)N([C@H](C)c1cnc(C(C)(C)O)cn1)C[C@H]3C ; C[C@@H]1Cc2nn3c(c2CN1C(=O)c1ccc(Cl)c(C(F)(F)F)c1)C(=O)N([C@H](C)c1cnc(C(C)(C)O)cn1)C[C@H]3C]

### Intermediate 41-2

### 5-Acetylpyrazine-2-carbonitrile

To the solution of 5-bromopyrazine-2-carbonitrile 41-1 (1.0 g, 5.44 mmol) in dry N,N-dimethylformamide (10 mL) were added tributyl(1-ethoxyvinyl)stannane (2.3 mL, 6.81 mmol) and bis(triphenylphosphine)palladium(II) chloride (85 mg, 0.12 mmol) at room temperature. The mixture was stirred at 85° C. for 16 hours. After cooling, the mixture was quenched with saturated potassium fluoride aqueous solution (50 mL) and extracted with ethyl acetate (50 mL) twice. The combined organic layers were washed with brine (50 mL), dried over Na₂SO₄₍ₛ₎ and filtered. The filtrate was concentrated to give the crude. The crude was dissolved in tetrahydrofuran (10 mL) and added 4 M hydrochloride in 1,4-dioxane (15 mL, 60 mmol). After stirred at room temperature for 3 hours, the mixture was concentrated and purified by silica gel column chromatography (petroleum ether:ethyl acetate=8:1) to give the compound (380 mg, 95% purity, 45.1% yield) as yellow solids. ¹H NMR (400 MHz, CDCl₃) δ 9.31 (d, J=1.6 Hz, 1H), 8.97 (d, J=1.2 Hz, 1H), 2.76 (s, 3H).

### Intermediate 41-3

### Ethyl (6R)-5-(4-chloro-3-(trifluoromethyl)benzoyl)-2-((2R)-1-((1-(5-cyanopyrazin-2-yl)ethyl)amino)propan-2-yl)-6-methyl-4,5,6,7-tetrahydro-2H-pyrazolo[4,3-c]pyridine-3-carboxylate

To a solution of ethyl (R)-2-((R)-1-aminopropan-2-yl)-5-(4-chloro-3-(trifluoromethyl)benzoyl)-6-methyl-4,5,6,7-tetrahydro-2H-pyrazolo[4,3-c]pyridine-3-carboxylate hydrochloride Int F (300 mg, 100% purity, 0.59 mmol) and 5-acetylpyrazine-2-carbonitrile 41-2 (100 mg, 100% purity, 0.68 mmol) in tetrahydrofuran (5 mL) were added tetraisopropoxytitanium (500 mg, 1.76 mmol) and triethylamine (60 mg, 0.59 mmol). The mixture was stirred at 70° C. for 3 hours. After sodium cyanoborohydride (80 mg, 1.27 mmol) was added at 0° C. and being stirred for 2 hours, the reaction was diluted with brine (20 mL), filtered and extracted with acetate (10 mL) for three times. The combined organic layers were washed with brine (10 mL), dried over Na₂SO₄₍ₛ₎, filtered and concentrated under vacuum to give a residue, which was purified by C18 chromatography (acetonitrile:water (+0.02% ammonium bicarbonate)=45-60%) to give the title compound (220 mg, 88% purity from LCMS, 54.4% yield) as yellow solids. LC-MS (ESI): R_{T}=1.79 min, mass calcd. for C₂₈H₂₉ClF₃N₇O₃ 603.2 m/z found 604.1 [M+H]⁺.

### Intermediate 41-4

### (6R)-5-(4-Chloro-3-(trifluoromethyl)benzoyl)-2-((2R)-1-((1-(5-(methoxycarbonyl) pyrazin-2-yl)ethyl)amino)propan-2-yl)-6-methyl-4,5,6,7-tetrahydro-2H-pyrazolo [4,3-c]pyridine-3-carboxylic acid

To a solution of ethyl (6R)-5-(4-chloro-3-(trifluoromethyl)benzoyl)-2-((2R)-1-((1-(5-cyanopyrazin-2-yl)ethyl)amino)propan-2-yl)-6-methyl-4,5,6,7-tetrahydro-2H-pyrazolo[4,3-c]pyridine-3-carboxylate 41-3 (220 mg, 88% purity, 0.32 mmol) in tetrahydrofuran (2 mL) and methanol (1 mL) was added lithium hydroxide hydrate (30 mg, 0.72 mmol) in water (1 mL) at 0° C. After stirred at 0° C. for 2 hours, the mixture was diluted with water (10 mL), acidized with 0.5 M hydrochloric acid aqueous solution to pH˜5 and extracted with ethyl acetate (10 mL) twice. The combined organic layers were washed with brine (10 mL), dried over Na₂SO₄₍ₛ₎ and filtered. The filtrate was concentrated to get the compound (220 mg, 76% purity from LCMS, 85.7% yield) as yellow solids. LC-MS (ESI): R_{T}=1.36 min, mass calcd. for C₂₇H₂₈ClF₃N₆O₅ 608.2 m/z found 609.1 [M+H]⁺.

### Intermediate 41-5

### Methyl 5-(1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)pyrazine-2-carboxylate

(6R)-5-(4-chloro-3-(trifluoromethyl)benzoyl)-2-((2R)-1-((1-(5-(methoxycarbonyl)pyrazin-2-yl)ethyl)amino)propan-2-yl)-6-methyl-4,5,6,7-tetrahydro-2H-pyrazolo[4,3-c]pyridine-3-carboxylic acid 41-4 (220 mg, 76% purity, 0.28 mmol) and 2-(3H-[1,2,3]triazolo[4,5-b]pyridin-3-yl)-1,1,3,3-tetramethylisouronium hexafluorophosphate(V) (180 mg, 0.47 mmol) were mixed in N,N-dimethylformamide (2 mL). After stirred at 0° C. for 30 min, triethylamine (100 mg, 0.99 mmol) in N, N-dimethylformamide (0.5 mL) was added dropwise at 0° C. After stirred at 0° C. for 2 hours, the reaction mixture was quenched with brine (10 mL), extracted with acetate (10 mL) for three times, dried over Na₂SO₄₍ₛ₎ and filtered. The filtrate was concentrated to get a residue, which was purified by C18 chromatography (acetonitrile:water (+0.02% ammonium bicarbonate)=40-60%) to give the title compound (110 mg, 93% purity from LCMS, 63.0% yield) as yellow solids. LC-MS (ESI): R_{T}=1.61 min, mass calcd. for C₂₇H₂₆ClF₃N₆O₄ 590.2 m/z found 591.6 [M+H]⁺.

### Compound 41

### (3R,7R)-2-(4-Chloro-3-(trifluoromethyl)benzoyl)-9-(1-(5-(2-hydroxypropan-2-yl)pyrazin-2-yl)ethyl)-3,7-dimethyl-1,2,3,4,8,9-hexahydropyrido[4′,3′:3,4]pyrazolo[1,5-a]pyrazin-10(7H)-one

To a solution of methyl 5-(1-((3R,7R)-2-(4-chloro-3-(trifluoromethyl)benzoyl)-3,7-dimethyl-10-oxo-1,3,4,7,8,10-hexahydropyrido[4′,3′:3,4]pyrazolo[1,5-a]pyrazin-9(2H)-yl)ethyl)pyrazine-2-carboxylate 41-5 (180 mg, 93% purity, 0.05 mmol) in tetrahydrofuran (3 mL) was added methylmagnesium bromide (3.0 M in tetrahydrofuran, 0.65 mL, 1.95 mmol) at −78° C. under nitrogen atmosphere. After stirred for 1 hour, the reaction mixture was quenched with saturated ammonium chloride solution (10 mL), extracted with acetate (10 mL) for three times, dried over Na₂SO₄₍ₛ₎, filtered. The filtrate was concentrated to get a residue, which was purified by prep. TLC (petroleum ether:ethyl acetate=1:2) to give the title compound (50 mg, 86% purity from LCMS, 38.5% yield) as yellow solids. LC-MS (ESI): R_{T}=1.60 min, mass calcd. for C₂₈H₃₀ClF₃N₆O₃ 590.2 m/z found 591.0.

### Compounds 41A and 41B

### (3R,7R)-2-(4-chloro-3-(trifluoromethyl)benzoyl)-9-((R*)-1-(5-(2-hydroxypropan-2-yl)pyrazin-2-yl)ethyl)-3,7-dimethyl-1,2,3,4,8,9-hexahydropyrido[4′,3′:3,4]pyrazolo[1,5-a]pyrazin-10(7H)-one (41A) and (3R,7R)-2-(4-chloro-3-(trifluoromethyl)benzoyl)-9-((S*)-1-(5-(2-hydroxypropan-2-yl)pyrazin-2-yl)ethyl)-3,7-dimethyl-1,2,3,4,8,9-hexahydropyrido[4′,3′:3,4]pyrazolo[1,5-a]pyrazin-10(7H)-one (41B)

The racemate of (3R,7R)-2-(4-chloro-3-(trifluoromethyl)benzoyl)-9-(1-(5-(2-hydroxypropan-2-yl)pyrazin-2-yl)ethyl)-3,7-dimethyl-1,2,3,4,8,9-hexahydropyrido[4′,3′:3,4]pyrazolo[1,5-a]pyrazin-10(7H)-one 41 (60 mg, 86% purity, 0.09 mmol) was separated by chiral prep HPLC (separation condition: Column: Chiralpak IC 5 μm 20*250 mm; Mobile Phase: Hex:EtOH=30:70 at 25 mL/min; Temp: 30° C.; Wavelength: 254 nm) to give the title compounds 41A (17.1 mg, 99.6% purity, 33.0% yield, 100% stereopure) as white solids and 41B (11.6 mg, 99.1% purity, 22.3% yield, 100% stereopure) as white solids.

### 41A:

LC-MS (ESI): R_{T}=3.175 min, mass calcd. for C₂₈H₃₀ClF₃N₆O₃ 590.2 m/z found 591.3 [M+H]⁺. Chiral analysis (Column: Chiralpak IC 5 μm 4.6*250 mm; Mobile Phase: Hex:EtOH=30:70 at 1 mL/min; Temp: 30° C.; Wavelength: 254 nm, R_{T}=5.648 min). ¹H NMR (400 MHz, DMSO-d₆) δ 8.85 (s, 1H), 8.65-8.51 (m, 1H), 7.91 (s, 1H), 7.85-7.78 (m, 2H), 5.95-5.72 (m, 1H), 5.52-5.16 (m, 2H), 4.58-4.36 (m, 2H), 4.22-4.06 (m, 1H), 3.97-3.78 (m, 1H), 3.42-3.32 (m, 1H), 2.97-2.92 (m, 1H), 2.66-2.51 (m, 1H), 1.70-1.52 (m, 3H), 1.49-1.41 (m, 6H), 1.28 (d, J=6.4 Hz, 3H), 1.23-1.05 (m, 3H). ¹⁹F NMR (376 MHz, DMSO-d₆) δ −61.32.

### 41B:

LC-MS (ESI): R_{T}=3.199 min, mass calcd. for C₂₈H₃₀ClF₃N₆O₃ 590.2 m/z found 591.3 [M+H]⁺. Chiral analysis (Column: Chiralpak IC 5 μm 4.6*250 mm; Mobile Phase: Hex:EtOH=30:70 at 1 mL/min; Temp: 30° C.; Wavelength: 254 nm, R_{T}=8.587 min). ¹H NMR (400 MHz, DMSO-d₆) δ 8.85 (s, 1H), 8.65-8.49 (m, 1H), 7.90 (s, 1H), 7.85-7.77 (m, 2H), 6.00-5.72 (m, 1H), 5.53-5.18 (m, 2H), 4.59-4.36 (m, 2H), 4.26-4.06 (m, 1H), 3.75-3.52 (m, 2H), 2.98-2.91 (m, 1H), 2.66-2.51 (m, 1H), 1.68-1.53 (m, 3H), 1.50-1.39 (m, 9H), 1.27-1.05 (m, 3H). ¹⁹F NMR (376 MHz, DMSO-d₆) δ −61.33.

### Compounds 42A and 42B

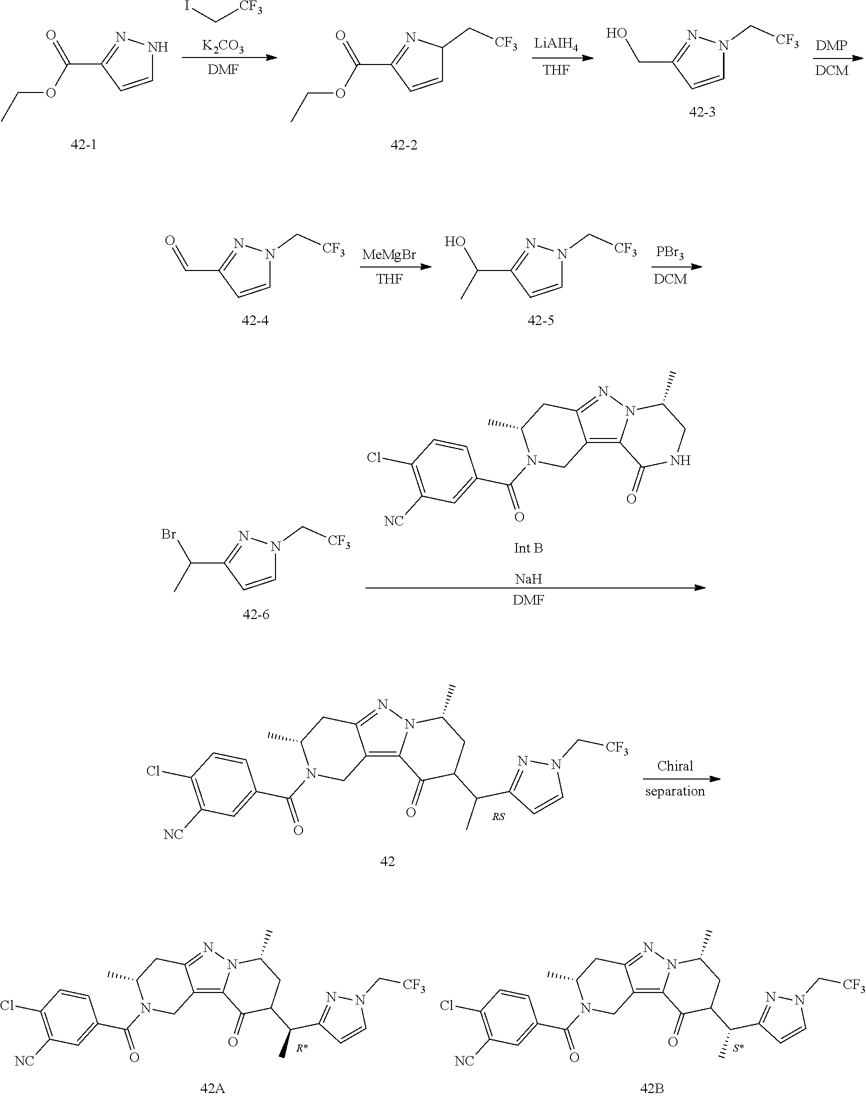

### Intermediate 42-2

### Ethyl 1-(2,2,2-trifluoroethyl)-1H-pyrazole-3-carboxylate

To a solution of ethyl pyrazole-3-carboxylate 42-1 (2 g, 14.3 mmol) and 2-iodo-1,1,1-trifluoroethane (2.4 mL, 24.4 mmol) in N,N-dimethylformamide (30 mL) was added potassium carbonate (6 g, 43.4 mmol). After stirred at 100° C. for 5 hours, the mixture was cooled down, diluted with water (80 mL), extracted with ethyl acetate (30 mL) twice. The combined organic layers were dried over Na₂SO₄₍ₛ₎, filtered. The filtrate was concentrated and purified by C18 column (acetonitrile:water=50% to 60%) to give the title compound (1.68 g, 100% purity from LCMS, 53% yield) as colorless oil. LC-MS (ESI): R_{T}=1.47 min, mass calcd. for C₈H₉F₃N₂O₂ 222.1, m/z found 223.0 [M+H]⁺.

### Intermediate 42-3

### (1-(2,2,2-Trifluoroethyl)-1H-pyrazol-3-yl)methanol

To a solution of ethyl 1-(2,2,2-trifluoroethyl)-1H-pyrazole-3-carboxylate 42-2 (1.6 g, 90% purity, 6.48 mmol) in tetrahydrofuran (20 mL) was added lithium aluminum hydride (300 mg, 7.90 mmol) at 0° C. After stirred at room temperature for 1 hour, the reaction was quenched with sodium sulfate decahydrate (800 mg), filtered. The filtrate was concentrated to give the title compound (1.1 g, 94% purity from LCMS, 89% yield) as colorless oil. LC-MS (ESI): R_{T}=0.96 min, mass calcd. for C₆H₇F₃N₂O 180.1, m/z found 181.0 [M+H]⁺.

### Intermediate 42-4

### 1-(2,2,2-Trifluoroethyl)-1H-pyrazole-3-carbaldehyde

To a solution of (1-(2,2,2-trifluoroethyl)-1H-pyrazol-3-yl)methanol 42-3 (1.1 g, 94% purity, 5.74 mmol) in dichloromethane (20 mL) was added Dess-martin periodinane (3 g, 7.07 mmol) at 0° C. After stirred at room temperature for 3 hours, the mixture was quenched with saturated sodium thiosulfate aqueous solution (20 mL), extracted with dichloromethane (20 mL) twice. The combined organic layers were washed with saturated sodium bicarbonate aqueous solution (20 mL) and brine (10 mL), dried over Na₂SO₄₍ₛ₎, filtered. The filtrate was concentrated to give the title compound (1.8 g, 50% purity from ¹H NMR, 88% yield) as white solids. ¹H NMR (400 MHz, CDCl₃) δ 10.00 (d, J=0.8 Hz, 1H), 7.59 (d, J=2.8 Hz, 1H), 6.90 (d, J=2.4 Hz, 1H), 4.82 (q, J=8.4 Hz, 2H).

### Intermediate 42-5

### 1-(1-(2,2,2-Trifluoroethyl)-1H-pyrazol-3-yl)ethan-1-ol

To a solution of 1-(2,2,2-trifluoroethyl)-1H-pyrazole-3-carbaldehyde 42-4 (1.8 g, 50% purity, 5.05 mmol) in tetrahydrofuran (20 mL) was added methylmagnesium bromide (3.6 mL, 3 M in 2-methyltetrahydrofuran, 10.8 mmol) at 0° C. under nitrogen atmosphere. Then the mixture was warmed to room temperature gradually and stirred for 3 hours. The reaction was quenched with water (10 mL), extracted with ethyl acetate (15 mL) for three times. The combined organic layers were dried over Na₂SO₄₍ₛ₎, filtered. The filtrate was concentrated and purified by C18 column (acetonitrile:water=20% to 40%) to give the title compound (570 mg, 90% purity from ¹H NMR, 52% yield) as colorless oil. ¹H NMR (400 MHz, CDCl₃) δ 7.45 (d, J=2.0 Hz, 1H), 6.32 (d, J=2.4 Hz, 1H), 4.96 (q, J=6.4 Hz, 1H), 4.66 (q, J=8.4 Hz, 2H), 2.30 (br s, 1H), 1.53 (d, J=6.8 Hz, 3H).

### Intermediate 42-6

### 3-(1-Bromoethyl)-1-(2,2,2-trifluoroethyl)-1H-pyrazole

To a solution of 1-(1-(2,2,2-trifluoroethyl)-1H-pyrazol-3-yl)ethan-1-ol 42-5 (500 mg, 90% purity, 2.32 mmol) in dichloromethane (5 mL) was added phosphorus tribromide (400 mg, 1.48 mmol) at 0° C. After stirred at room temperature for 1 hour, the mixture was quenched with saturated sodium bicarbonate solution (10 mL), extracted with dichloromethane (10 mL) twice. The combined organic layers were washed with brine (5 mL), dried over Na₂SO₄₍ₛ₎, filtered. The filtrate was concentrated to give the title compound (640 mg, 90% purity from ¹H NMR, 97% yield) as brown oil. ¹H NMR (400 MHz, CDCl₃) δ 7.45 (d, J=1.6 Hz, 1H), 6.44 (d, J=2.4 Hz, 1H), 5.27 (q, J=7.2 Hz, 1H), 4.66 (dq, J=8.4, 1.2 Hz, 2H), 2.05 (d, J=6.8 Hz, 3H).

### Compound 42

### 2-chloro-5-((3R,7R)-3,7-dimethyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-2-carbonyl)benzonitrile

To a solution of (2-chloro-5-((3R,7R)-3,7-dimethyl-10-oxo-1,2,3,4,7,8,9,10-octahydropyrido[4′,3′:3,4]pyrazolo[1,5-a]pyrazine-2-carbonyl)benzonitrile Int B (200 mg, 100% purity, 0.521 mmol) in dry N,N-dimethylformamide (5 mL) was added 60% sodium hydride in mineral oil (66 mg, 1.65 mmol) slowly at 0° C. After stirred at 0° C. for 20 minutes, the 3-(1-bromoethyl)-1-(2,2,2-trifluoroethyl)-1H-pyrazole 42-6 (280 mg, 90% purity, 0.98 mmol) was added dropwise and the mixture was stirred at 0° C. for 2 hours. The reaction mixture was quenched with water (20 mL) and extracted with ethyl acetate (20 mL) twice. The combined organic layers were washed with brine (25 mL), dried over Na₂SO₄₍ₛ₎ and filtered. The filtrate was concentrated and purified by C18 column (acetonitrile:water=50% to 60%) to give the title compound (120 mg, 90% purity from ¹H NMR, 41.2% yield) as white solids. LC-MS (ESI): R_{T}=1.58 min, mass calcd. for C₂₆H₂₅ClF₃N₇O₂ 559.2, m/z found 560.1 [M+H]⁺.

### Compounds 42A and 42B

### 2-chloro-5-((3R,7R)-3,7-Dimethyl-10-oxo-9-((R*)-1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-2-carbonyl)benzonitrile (42A) and 2-chloro-5-((3R,7R)-3,7-dimethyl-10-oxo-9-((S*)-1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-2-carbonyl)benzonitrile (42B)

The racemate of 2-chloro-5-((3R,7R)-3,7-dimethyl-10-oxo-9-(1-(1-(2,2,2-trifluoroethyl)-1H-pyrazol-3-yl)ethyl)-1,2,3,4,7,8,9,10-octahydropyrido[4′,3′:3,4]pyrazolo[1,5-a]pyrazine-2-carbonyl)benzonitrile 42 (140 mg, 90% purity, 0.329 mmol) was separated by chiral Prep. (Column: Chiralpak IC 5 μm 30*250 mm; Mobile Phase: EtOH:EtOH=50:50 at 15 mL/min; Col. Temp: 30° C.; Wavelength: 254 nm) to give the compounds 42A (16.1 mg, 99.6% purity, 12.7% yield, 100% stereopure) as white solids and 42B (17.9 mg, 99.7% purity, 14.2% yield, 99.7% stereopure) as white solids.

### 42A:

LC-MS (ESI): R_{T}=3.772 min, mass calcd. for C₂₆H₂₅ClF₃N₇O₂ 559.2, m/z found 560.3 [M+H]⁺. Chiral analysis (Column: Chiralpak IC 5 μm 4.6*250 mm; Mobile Phase: MeOH:EtOH=50:50 at 1 mL/min; Temp: 30° C.; Wavelength: 254 nm; Rt=6.485 min). ¹H NMR (400 MHz, CDCl₃) δ 7.74 (s, 1H), 7.61-7.56 (m, 2H), 7.49-7.45 (m, 1H), 6.36-5.98 (m, 2H), 5.81-5.34 (m, 1H), 4.82-4.39 (m, 5H), 3.75-3.30 (m, 2H), 3.15-2.66 (m, 2H), 1.56-1.50 (m, 3H), 1.32-1.21 (m, 6H). ¹⁹F NMR (376 MHz, CDCl₃) δ −71.78.

### 42B:

LC-MS (ESI): R_{T}=3.817 min, mass calcd. for C₂₆H₂₅ClF₃N₇O₂ 559.2, m/z found 560.3 [M+H]⁺. Chiral analysis (Column: Chiralpak IC 5 μm 4.6*250 mm; Mobile Phase: MeOH:EtOH=50:50 at 1 mL/min; Temp: 30° C.; Wavelength: 254 nm; Rt=7.63 min). ¹H NMR (400 MHz, CDCl₃) δ 7.74 (s, 1H), 7.61-7.56 (m, 2H), 7.48-7.42 (m, 1H), 6.33-6.19 (m, 1H), 6.10-5.89 (m, 1H), 5.55-5.20 (m, 1H), 4.72-4.65 (m, 3H), 4.50-4.23 (m, 2H), 3.58-3.47 (m, 1H), 3.41-3.32 (m, 1H), 3.17-2.91 (m, 1H), 2.76-2.67 (m, 1H), 1.65-1.57 (m, 3H), 1.53-1.50 (m, 3H), 1.34-1.22 (m, 3H). ¹⁹F NMR (376 MHz, CDCl₃) δ −71.83.

### Compounds 43A and 43B

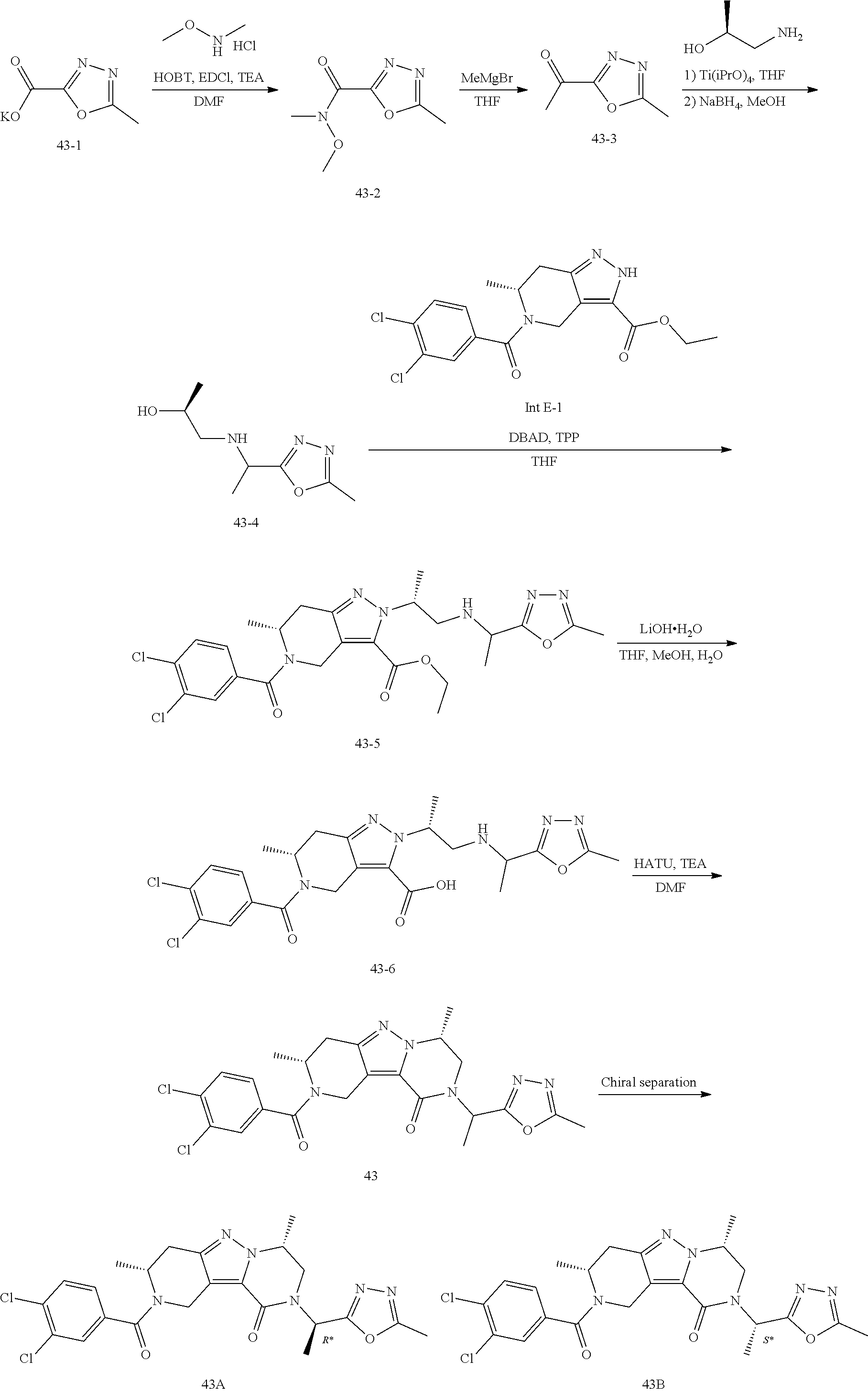

### Intermediate 43-2

### N-Methoxy-N,5-dimethyl-1,3,4-oxadiazole-2-carboxamide

To a solution of 5-Methyl-1,3,4-oxadiazole-2-carboxylic acid potassium salt 43-1 (11.0 g, 66.2 mmol), N,O-dimethylhydroxylamine hydrochloride (9.70 μg, 99.4 mmol), 1H-benzo[d][1,2,3]triazol-1-ol (13.5 g, 99.9 mmol) and triethylamine (13.8 g, 136 mmol) in N,N-dimethylformamide (100 mL) was added 1-ethyl-3-(3-dimethylaminopropyl)carbodiimide (19.0 g, 99.1 mmol) at room temperature under nitrogen atmosphere. After stirred at room temperature overnight, the mixture was diluted with water (120 mL) and extracted with ethyl acetate (130 mL) for three times. The combined organic layers were washed with brine (10 mL) for three times, dried over Na₂SO₄₍ₛ₎ and filtered. The filtrate was concentrated to give the title compound (9.00 g, 70% purity from ¹H NMR, 56% yield) as light yellow oil. ¹H NMR (400 MHz, CDCl₃) δ 3.89 (s, 3H), 3.37 (br s, 3H), 2.61 (s, 3H).

### Intermediate 43-3

### 1-(5-Methyl-1,3,4-oxadiazol-2-yl)ethan-1-one

To the solution of N-methoxy-N,5-dimethyl-1,3,4-oxadiazole-2-carboxamide 43-2 (7.00 g, 70% purity, 28.6 mmol) in tetrahydrofuran (50 mL) was added 1 M methylmagnesium bromide in tetrahydrofuran (30 mL, 30 mmol) at 0° C. After stirred at 0° C. for 1 hour, the mixture was quenched with ammonium chloride aqueous solution (10 mL) and extracted with ethyl acetate (10 mL) twice. The combined organic layers were washed with brine (10 mL), dried over Na₂SO₄₍ₛ₎ and filtered. The filtrate was concentrated and purified by silica gel column chromatography (petroleum ether:ethyl acetate=3:1) to give the title compound (2.70 g, 90% purity from ¹H NMR, 67% yield) as yellow oil. ¹H NMR (400 MHz, CDCl₃) δ 2.72 (d, J=11.6 Hz, 3H), 2.62 (d, J=9.2 Hz, 3H).

### Intermediate 43-4

### (2S)-1-((1-(5-Methyl-1,3,4-oxadiazol-2-yl)ethyl)amino)propan-2-ol

To a solution of 1-(5-methyl-1,3,4-oxadiazol-2-yl)ethan-1-one 43-3 (1.00 g, 90% purity, 7.14 mmol) and (S)-1-aminopropan-2-ol (1.60 g, 21.3 mmol) in tetrahydrofuran (10 mL) was added titanium tetraisopropanolate (4 mL, 13.7 mmol). After stirred at 70° C. for 24 hours, the reaction mixture was cooled and concentrated and purified by silica gel column chromatography (petroleum ether:ethyl acetate=3:1) to give (S)-1-((1-(5-methyl-1,3,4-oxadiazol-2-yl)ethylidene)amino)propan-2-ol (900 mg, 100% purity from LCMS, 69% yield) as yellow oil. To a solution of the Intermediate imine (900 mg, 100% purity, 4.91 mmol) in methanol (10 mL) was added dropwise sodium borohydride (100 mg, 2.64 mmol) at 0° C. After stirred at 0° C. for 2 hours under nitrogen atmosphere, the reaction mixture was acidified with 1 M hydrochloride aqueous solution to pH˜5 and extracted with dichloromethane (10 mL) for three times. The combined organic layers were washed with brine (20 mL), dried over Na₂SO₄₍ₛ₎ and filtered. The filtrate was concentrated to give a residue, which was purified by silica gel column chromatography (petroleum ether:ethyl acetate=10:1 to 2:1) to afford the title compound (800 mg, 100% purity from LCMS, 88% yield) as white solids. LC-MS (ESI): R_{T}=0.32 min, mass calcd. for C₈H₁₅N₃O₂ 185.1, m/z found 186.4 [M+H]⁻.

### Intermediate 43-5

### (6R)-Ethyl 5-(3,4-Dichlorobenzoyl)-6-methyl-2-((2R)-1-((1-(5-methyl-1,3,4-oxadiazol-2-yl)ethyl)amino)propan-2-yl)-4,5,6,7-tetrahydro-2H-pyrazolo[4,3-c]pyridine-3-carboxylate

To a solution of triphenylphosphine (1.10 g, 4.19 mmol), (2S)-1-((1-(5-methyl-1,3,4-oxadiazol-2-yl)ethyl)amino)propan-2-ol 43-4 (400 mg, 100% purity, 2.16 mmol) and (R)-ethyl 5-(3,4-dichlorobenzoyl)-6-methyl-4,5,6,7-tetrahydro-2H-pyrazolo[4,3-c]pyridine-3-carboxylate Int E-1 (800 mg, 95% purity, 1.99 mmol) in dry tetrahydrofuran (10 mL) was added di-tert-butyl diazene-1,2-dicarboxylate (995 mg, 4.32 mmol) at 0° C. under nitrogen atmosphere. The resulting mixture was stirred at 25° C. overnight. The reaction was poured into water (20 mL), extracted with ethyl acetate (20 mL) for three times. The combined organic layers were washed with brine (20 mL), dried over Na₂SO₄₍ₛ₎ and filtered. The filtrate was concentrated in vacuo. The residue was purified by column chromatography on silica gel (petroleum ether:ethyl acetate=10:1 to 2:1) to give the title product (380 mg, 100% purity from LCMS, 34% yield) as white solids. LC-MS (ESI): R_{T}=1.63 min, mass calcd. for C₂₅H₃₀Cl₂N₆O₄ 548.2, m/z found 549.5 [M+H]⁺.

### Intermediate 43-6

### (6R)-5-(3,4-Dichlorobenzoyl)-6-methyl-2-((2R)-1-((1-(5-methyl-1,3,4-oxadiazol-2-yl)ethyl)amino)propan-2-yl)-4,5,6,7-tetrahydro-2H-pyrazolo[4,3-c]pyridine-3-carboxylic acid

To a solution of (6R)-ethyl 5-(3,4-dichlorobenzoyl)-6-methyl-2-((2R)-1-((1-(5-methyl-1,3,4-oxadiazol-2-yl)ethyl)amino)propan-2-yl)-4,5,6,7-tetrahydro-2H-pyrazolo[4,3-c]pyridine-3-carboxylate 43-5 (380 mg, 100% purity, 0.692 mmol) in tetrahydrofuran (2 mL), methanol (4 mL) and water (2 mL) was added lithium hydroxide monohydrate (60 mg, 1.43 mmol) under nitrogen atmosphere. After stirred at room temperature overnight, the reaction mixture was concentrated at 35° C. to give a residue, which was purified by C18 column (acetonitrile:water=30% to 90%) to give the desired compound (300 mg, 100% purity from LCMS, 83% yield) as light yellow solids. LC-MS (ESI): R_{T}=1.22 min, mass calcd. for C₂₃H₂₆Cl₂N₆O₄ 520.1, m/z found 521.3 [M+H]⁺.

### Compound 43

### (3R,7R)-2-(3,4-Dichlorobenzoyl)-3,7-dimethyl-9-(1-(5-methyl-1,3,4-oxadiazol-2-yl)ethyl)-1,2,3,4,8,9-hexahydropyrido[4′,3′:3,4]pyrazolo[1,5-a]pyrazin-10(7H)-one

To a solution of (6R)-5-(3,4-dichlorobenzoyl)-6-methyl-2-((2R)-1-((1-(5-methyl-1,3,4-oxadiazol-2-yl)ethyl)amino)propan-2-yl)-4,5,6,7-tetrahydro-2H-pyrazolo[4,3-c]pyridine-3-carboxylic acid 43-6 (300 mg, 100% purity, 0.575 mmol) in N,N-dimethylformamide (10 mL) were added triethylamine (190 mg, 1.88 mmol) and 2-(7-azabenzotriazol-1-yl)-N,N,N′,N′-tetramethyluronium hexafluorophosphate (330 mg, 0.868 mmol) at room temperature. After stirred at room temperature for 2 hours, water (20 mL) was added and extracted with ethyl acetate (10 mL) for three times. The combined organic layers were washed with brine (20 mL), dried over Na₂SO₄₍ₛ₎ and concentrated to give a residue, which was purified by silica gel column chromatography (petroleum ether:ethyl acetate=5:1 to 1:1) to give the title compound (200 mg, 95% purity from LCMS, 65% yield) as white solids. LC-MS (ESI): R_{T}=1.326 min, mass calcd. for C₂₃H₂₄Cl₂N₆O₃ 502.1, m/z found 503.0 [M+H]⁺.

### Compounds 43A and 43B

### (3R,7R)-2-(3,4-dichlorobenzoyl)-3,7-dimethyl-9-((R*)-1-(5-methyl-1,3,4-oxadiazol-2-yl)ethyl)-1,2,3,4,8,9-hexahydropyrido[4′,3′:3,4]pyrazolo[1,5-a]pyrazin-10(7H)-one (43A) and (3R,7R)-2-(3,4-dichlorobenzoyl)-3,7-dimethyl-9-((S*)-1-(5-methyl-1,3,4-oxadiazol-2-yl)ethyl)-1,2,3,4,8,9-hexahydropyrido[4′,3′:3,4]pyrazolo[1,5-a]pyrazin-10(7H)-one (43B)

A racemic mixture of (3R,7R)-2-(3,4-dichlorobenzoyl)-3,7-dimethyl-9-(1-(5-methyl-1,3,4-oxadiazol-2-yl)ethyl)-1,2,3,4,8,9-hexahydropyrido[4′,3′:3,4]pyrazolo[1,5-a]pyrazin-10(7H)-one 43 (280 mg, 95% purity, 0.528 mmol) was separated by chiral Prep. HPLC (separation condition: Column: Chiralpak IC 5 μm 30*250 mm; Mobile Phase: ACN:IPA=70:30 at 25 mL/min; Temp: 30° C.; Wavelength: 214 nm) to give the title compounds 43A (28.3 mg, 99.7% purity from LCMS, 11% yield, 100% stereopure) and 43B (50.1 mg, 99.8% purity from LCMS, 19% yield, 100% stereopure) as white solids.

### 43A:

LC-MS (ESI): R_{T}=3.026 min, mass calcd. for C₂₃H₂₄Cl₂N₆O₃ 502.1, m/z found 503.2 [M+H]⁺. Chiral analysis (Column: Chiralpak IC 5 μm 4.6*250 mm; Mobile Phase: ACN:IPA=70:30 at 1 mL/min; Temp: 30° C.; Wavelength: 254 nm). ¹H NMR (400 MHz, CDCl₃) δ 7.55-7.47 (m, 2H), 7.25-7.21 (m, 1H), 6.28-5.99 (m, 1H), 5.81-5.25 (m, 1H), 4.91-4.13 (m, 3H), 3.89-3.68 (m, 1H), 3.52-3.33 (m, 1H), 3.18-2.86 (m, 1H), 2.74-2.58 (m, 1H), 2.56-2.48 (m, 3H), 1.74-1.64 (m, 3H), 1.49-1.41 (m, 3H), 1.32-1.18 (m, 3H).

### 43B:

LC-MS (ESI): R_{T}=3.118 min, mass calcd. for C₂₃H₂₄Cl₂N₆O₃ 502.1, m/z found 503.1 [M+H]⁺. Chiral analysis (Column: Chiralpak IC 5 μm 4.6*250 mm; Mobile Phase: ACN:IPA=70:30 at 1 mL/min; Temp: 30° C.; Wavelength: 254 nm). ¹H NMR (400 MHz, CDCl₃) δ 7.58-7.45 (m, 2H), 7.26-7.22 (m, 1H), 6.25-6.01 (m, 1H), 5.81-5.30 (m, 1H), 4.96-4.17 (m, 3H), 3.74-3.63 (m, 1H), 3.54-3.38 (m, 1H), 3.19-2.85 (m, 1H), 2.76-2.60 (m, 1H), 2.56-2.47 (m, 3H), 1.73-1.65 (m, 3H), 1.60 (d, J=6.6 Hz, 3H), 1.31-1.19 (m, 3H).

### Compounds 44A and 44B

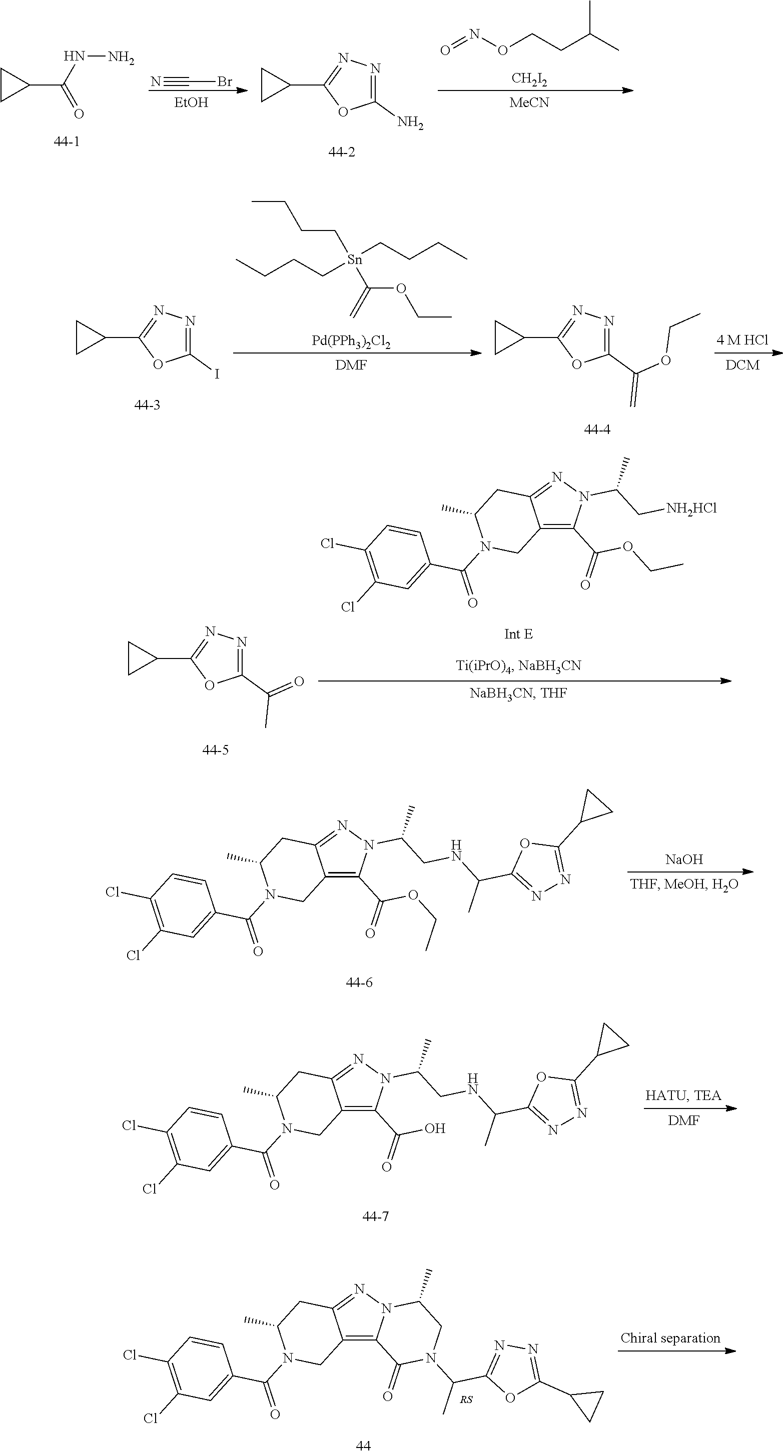

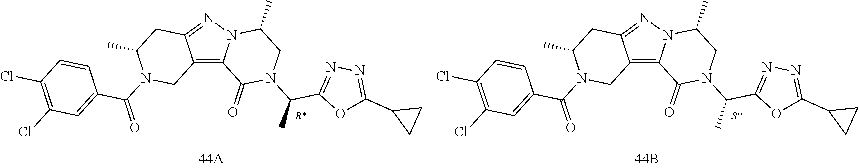 [SMILES: C[C@@H]1Cc2nn3c(c2CN1C(=O)c1ccc(Cl)c(Cl)c1)C(=O)N([C@H](C)c1nnc(C2CC2)o1)C[C@H]3C ; C[C@@H]1Cc2nn3c(c2CN1C(=O)c1ccc(Cl)c(Cl)c1)C(=O)N([C@@H](C)c1nnc(C2CC2)o1)C[C@H]3C]

### Intermediate 44-2

### 5-Cyclopropyl-1,3,4-oxadiazol-2-amine

To the solution of cyclopropanecarbohydrazide 44-1 (5 g, 49.9 mmol) in ethanol (100 mL) was added cyanogen bromide (10.5 g, 99.1 mmol) at 0° C. After stirred at 60° C. for 3 hours, the solution was poured into saturated sodium bicarbonate aqueous solution (100 mL), concentrated, filtered to give yellow crude A. The filtrate was extracted with ethyl acetate (100 mL) for three times. The combined organic layers were dried over Na₂SO₄₍ₛ₎, filtered, concentrated to give yellow residue, which was combined with crude A and triturated with dichloromethane (50 mL) and diethyl ether (50 mL) to give the title compound (4.9 g, 80% purity from ¹H NMR, 63% yield) as yellow solids. ¹H NMR (DMSO-d₆) δ: 6.90-6.68 (m, 2H), 2.06-1.87 (m, 1H), 1.01-0.92 (m, 2H), 0.91-0.73 (m, 2H).

### Intermediate 44-3

### 2-Cyclopropyl-5-iodo-1,3,4-oxadiazole

To the solution of 5-cyclopropyl-1,3,4-oxadiazol-2-amine 44-2 (4.9 g, 80% purity, 31.3 mmol) in acetonitrile (100 mL) was added diiodomethane (5.4 mL, 67.1 mmol) and isoamyl nitrite (15 mL, 111 mmol). After stirred at 70° C. overnight, the solution was cooled down, diluted with water (50 mL), concentrated to remove acetonitrile, extracted with ethyl acetate (50 mL) for three times. The combined organic layers were washed with brine (50 mL), dried over Na₂SO₄₍ₛ₎ and filtered. The filtrate was concentrated, purified by C18 column (acetonitrile:water=30% to 50%) to give the title compound (4.7 g, 84% purity from LCMS, 53% yield) as yellow solids. LC-MS (ESI): R_{T}=1.27 min, mass calcd. for C₅H₅IN₂O 235.9, m/z found 236.9 [M+H]⁺.

### Intermediate 44-4

### 2-Cyclopropyl-5-(1-ethoxyvinyl)-1,3,4-oxadiazole

To the solution of 2-cyclopropyl-5-iodo-1,3,4-oxadiazole 44-3 (4.7 g, 80% purity, 15.9 mmol) and tributyl(1-ethoxyvinyl)tin (6.6 mL, 19.5 mmol) in N,N-dimethylformamide (50 mL) was added bis(triphenylphosphine)palladium(II) chloride (1.2 g, 1.71 mmol). After stirred at 85° C. under nitrogen atmosphere for 5 hours, the solution was cooled down, quenched with aqueous solution of potassium fluoride (40 mL), diluted with water (100 mL), extracted with ethyl acetate (60 mL) for three times. The combined organic layers were washed with brine (60 mL), dried over Na₂SO₄₍ₛ₎, filtered. The filtrate was concentrate and purified by C18 column (acetonitrile:water=40% to 60%) to give the title compound (2 g, 100% purity from LCMS, 70% yield) as brown oil. LC-MS (ESI): R_{T}=1.40 min, mass calcd. for C₉H₁₂N₂O₂ 180.1, m/z found 181.1 [M+H]⁺.

### Intermediate 44-5

### 1-(5-Cyclopropyl-1,3,4-oxadiazol-2-yl)ethan-1-one

To the solution of 2-cyclopropyl-5-(1-ethoxyvinyl)-1,3,4-oxadiazole 44-4 (1.8 g, 100% purity, 9.99 mmol) in dichloromethane (9 mL) was added the solution of 4 M hydrochloride aqueous solution (9 mL, 36.0 mmol) in 1,4-dioxane. After stirred at room temperature for 30 minutes, the solution was alkalified with sodium bicarbonate saturated aqueous solution (20 mL), extracted with dichloromethane (20 mL) twice. The combined organic layers were washed with brine (20 mL), dried over Na₂SO₄₍ₛ₎, filtered. The filtrate was concentrated and purified by silica gel column chromatography (petroleum ether:ethyl acetate=15:1) to give the title compound (1.4 g, 95% purity from ¹H NMR, 88% yield) as colorless oil. LC-MS (ESI): R_{T}=1.02 min, mass calcd. for C₇H₈N₂O₂ 152.1, m/z found 153.0 [M+H]⁺.

### Intermediate 44-6

### (6R)-Ethyl 2-((2R)-1-((1-(5-cyclopropyl-1,3,4-oxadiazol-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylate

To a solution of (R)-ethyl 2-((R)-1-aminopropan-2-yl)-5-(3,4-dichlorobenzoyl)-6-methyl-4,5,6,7-tetrahydropyrazolo[1,5-a]pyrazine-3-carboxylate hydrochloride Int E (500 mg, 95% purity, 0.998 mmol) and 1-(5-cyclopropyl-1,3,4-oxadiazol-2-yl)ethanone 44-5 (250 mg, 95% purity, 1.56 mmol) in tetrahydrofuran (7 mL) was added titanium tetraisopropanolate (0.5 mL, 1.69 mmol). After stirred at room temperature for 2 hours, the reaction was cooled down to 0° C., sodium cyanoborohydride (125 mg, 1.99 mmol) was added, stirred at room temperature for 2 hours. The solution was quenched with water (10 mL), stirred for 10 minutes, filtered. The filtrate was extracted with ethyl acetate (10 mL) twice. The combined organic layers were washed with brine (10 mL), dried over Na₂SO₄₍ₛ₎, filtered, concentrated, purified by C18 column (acetonitrile:water=50% to 60%) to give the title compound (400 mg, 98% purity from LCMS, 68% yield) as colorless oil. LC-MS (ESI): R_{T}=1.71 min, mass calcd. for C₂₇H₃₂Cl₂N₆O₄ 574.2, m/z found 575.1 [M+H]⁺.

### Intermediate 44-7

### (6R)-2-((2R)-1-((1-(5-cyclopropyl-1,3,4-oxadiazol-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid

To the solution of sodium hydroxide monohydrate (80 mg, 1.91 mmol) in water (2 mL) and methanol (2 mL) was added the solution of (6R)-ethyl 2-((2R)-1-((1-(5-cyclopropyl-1,3,4-oxadiazol-2-yl)ethyl)amino)propan-2-yl)-5-(3,4-dichlorobenzoyl)-6-methyl-4,5,6,7-tetrahydropyrazolo[1,5-a]pyrazine-3-carboxylate 44-6 (500 mg, 98% purity, 0.851 mmol) in tetrahydrofuran (6 mL). After stirred at room temperature for 1 hour, the mixture was diluted with water (5 mL), acidized with 1 N hydrochloride to pH 4, extracted with ethyl acetate (10 mL) for three times. The combined organic layers were dried over Na₂SO₄₍ₛ₎, concentrated to give the title compound (450 mg, 100% purity from LCMS, 97% yield) as white solids. LC-MS (ESI): R_{T}=1.29 min, mass calcd. for C₂₅H₂₈Cl₂N₆O₄ 546.2, m/z found 547.1 [M+H]⁺.

### Compound 44

### (3R,7R)-9-(1-(5-cyclopropyl-1,3,4-oxadiazol-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To the solution of (6R)-2-((2R)-1-((1-(5-cyclopropyl-1,3,4-oxadiazol-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid 44-7 (450 mg, 100% purity, 0.822 mmol) in N,N-dimethylformamide (20 mL) were added triethylamine (170 mg, 1.68 mmol) and O-(7-azabenzotriazol-1-yl)-N,N,N′,N′-tetramethyluronium hexafluorophosphate (470 mg, 1.24 mmol). After stirred at room temperature for 1 hour, the mixture was diluted with water (50 mL), extracted with ethyl acetate (20 mL) twice. The combined organic layers were washed with brine (20 mL), dried over Na₂SO₄₍ₛ₎, filtered. The filtrate was concentrated, purified by C18 column (acetonitrile:water=50% to 60%) to give the title compound (300 mg, 100% purity from LCMS, 69% yield) as white solids. LC-MS (ESI): R_{T}=1.58 min, mass calcd. for C₂₅H₂₆Cl₂N₆O₃ 528.1, m/z found 529.1 [M+H]⁺.

### Compounds 44A and 44B

### (3R,7R)-9-((R*)-1-(5-cyclopropyl-1,3,4-oxadiazol-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (44A) and (3R,7R)-9-((S*)-1-(5-cyclopropyl-1,3,4-oxadiazol-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (44B)

A racemate of (3R,7R)-9-(1-(5-cyclopropyl-1,3,4-oxadiazol-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 44 (300 mg, 100% purity, 0.567 mmol) was separated by chiral Prep. HPLC (separation condition: Column: Chiralpak IE 5 m 30*250 mm; Mobile Phase: ACN:IPA=50:50 at 25 mL/min; Col. Temp: 30° C. Wavelength: 254 nm) to afford the title compounds 44A (150 mg, 99.1% purity from LCMS, 50% yield, 100% stereopure) as white solids and 44B (92 mg, 99.5% purity from LCMS, 31% yield, 99.9% stereopure) as white solids.

### 44A:

LC-MS (ESI): R_{T}=3.720 min, mass calcd. for C₂₅H₂₆Cl₂N₆O₃ 528.1, m/z found 529.1 [M+H]⁺. Chiral HPLC (Column: Chiralpak IE 5 m 4.6*250 mm; Mobile Phase: ACN:IPA=50:50 at 1 mL/min; Temp: 30° C.; Wavelength: 254 nm), R_{T}=5.740 min). ¹H NMR (400 MHz, CDCl₃) δ 7.53-7.49 (m, 2H), 7.26-7.24 (m, 1H), 6.10 (s, 1H), 5.70-5.40 (m, 1H), 4.81-4.30 (m, 3H), 3.81-3.78 (m, 1H), 3.39 (dd, J=13.2 and 6.0 Hz, 1H), 3.04 (br s, 1H), 2.67 (d, J=15.2 Hz, 1H), 2.16-2.09 (m, 1H), 1.66 (d, J=6.8 Hz, 3H), 1.42 (d, J=6.8 Hz, 3H), 1.26 (d, J=4.4 Hz, 3H), 1.16-1.09 (m, 4H).

### 44B:

LC-MS (ESI): R_{T}=3.713 min, mass calcd. for C₂₅H₂₆Cl₂N₆O₃ 528.1, m/z found 529.2 [M+H]⁺. Chiral HPLC (Column: Chiralpak IE 5 m 4.6*250 mm; Mobile Phase: ACN:IPA=50:50 at 1 mL/min; Temp: 30° C.; Wavelength: 254 nm), R_{T}=8.566 min). ¹H NMR (400 MHz, CDCl₃) δ 7.53-7.49 (m, 2H), 7.26-7.24 (m, 1H), 6.10-5.41 (m, 2H), 4.85-4.30 (m, 3H), 3.68 (dd, J=12.8 and 4.4 Hz, 1H), 3.46 (dd, J=12.8 and 8.0 Hz, 1H), 3.05 (br s, 1H), 2.68 (d, J=16.0 Hz, 1H), 2.15-2.08 (m, 1H), 1.66 (d, J=7.2 Hz, 3H), 1.60-1.58 (m, 3H), 1.25 (s, 3H), 1.15-1.10 (m, 4H).

### Compounds 45A and 45B

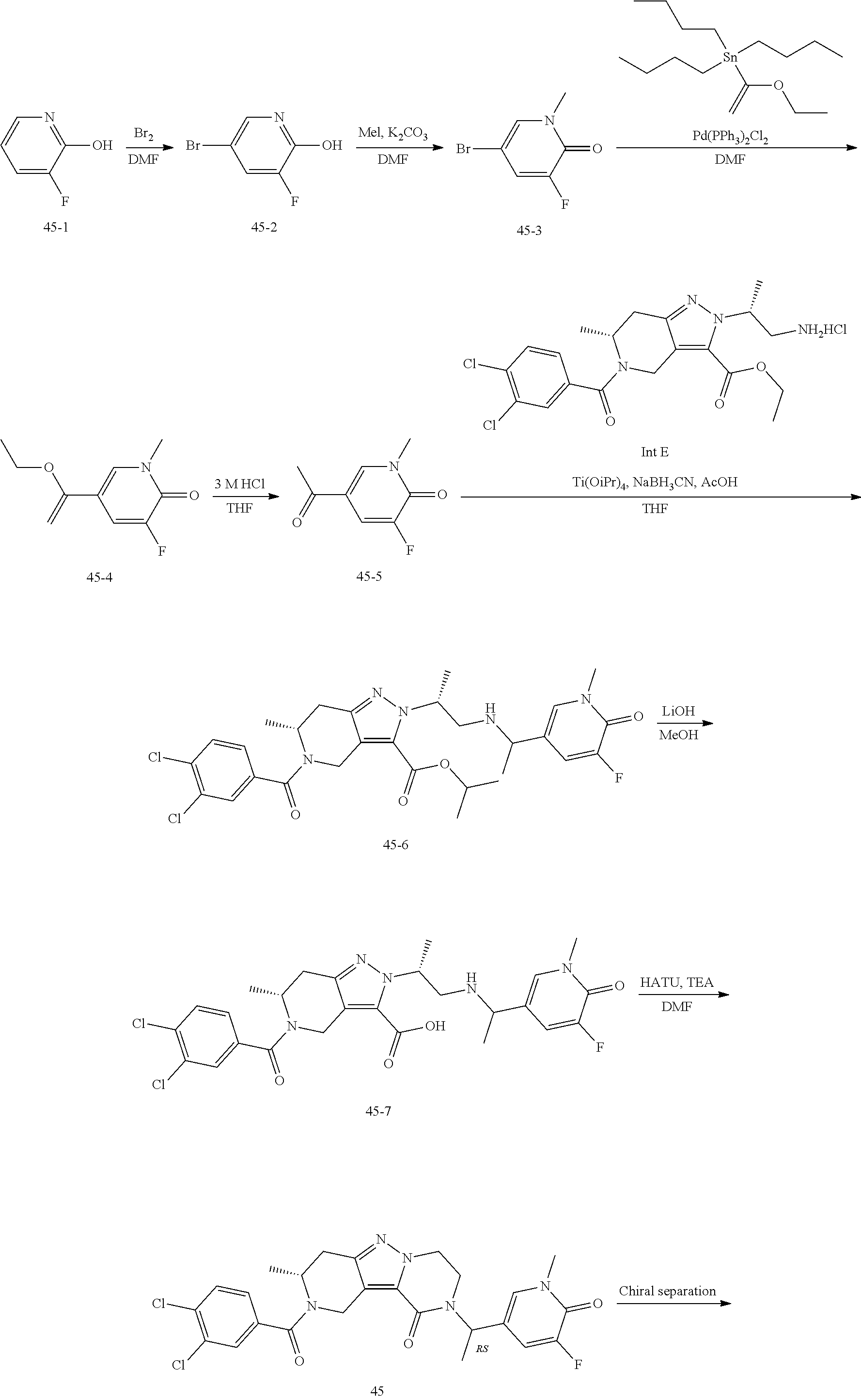

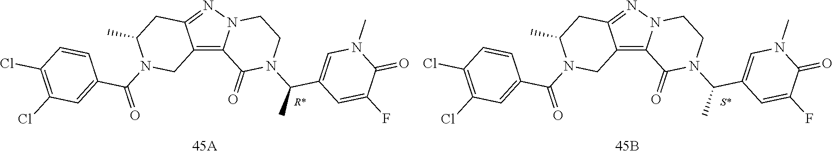

### Intermediate 45-2

### 5-Bromo-3-fluoropyridin-2-ol

To a solution of 3-fluoropyridin-2-ol 45-1 (3.0 g, 26.5 mmol) in N,N-dimethylformamide (33 mL) was added dropwise bromine (1.5 mL, 29.3 mmol) at 0° C. The reaction was gradually warmed to room temperature for 12 hours, the reaction solution was quenched with saturated sodium thiosulfate (20 mL), extracted with dichloromethane (50 mL) for three times and the combined organic layers were washed with brine (100 mL), dried over Na₂SO₄₍ₛ₎ and filtered. The filtrate was concentrated to give the title compound (3.9 g, 60% purity from LCMS, 45.9% yield) as yellow solids. LC-MS (ESI): R_{T}=0.32 min, mass calcd. for C₅H₃BrFNO 190.9 m/z found 191.9 [M+H]⁺.

### Intermediate 45-3

### 5-Bromo-3-fluoro-1-methylpyridin-2(1H)-one

The mixture of 5-bromo-3-fluoropyridin-2-ol 45-2 (1.2 g, 60% purity, 3.75 mmol), iodomethane (800 mg, 5.64 mmol) and potassium carbonate (1.1 g, 7.96 mmol) in N,N-dimethylformamide (15 mL) was stirred at 30° C. for 12 hours. The mixture was treated with water (20 ml) and extracted with acetate (30 mL) for three times and the combined organic layers were washed with brine (30 mL), dried over Na₂SO₄₍ₛ₎ and filtered. The filtrate was concentrated in vacuum to give the title compound (1 g, 77% purity from LCMS, 99.7% yield) as yellow solids. LC-MS (ESI): R_{T}=1.03 min, mass calcd. for C₆H₅BrFNO 204.9 m/z found 206.0 [M+H]⁺.

### Intermediate 45-4

### 5-(1-Ethoxyvinyl)-3-fluoro-1-methylpyridin-2(1H)-one

To a solution of 5-bromo-3-fluoro-1-methylpyridin-2(1H)-one 45-3 (1 g, 77% purity, 3.74 mmol) and tributyl(1-ethoxyethenyl)stannane (2 g, 5.54 mmol) in N,N-dimethylformamide (20 mL) was added bis(triphenylphosphine)palladium(II) dichloride (260 mg, 0.37 mmol) under nitrogen atmosphere. After stirred at 100° C. for 4 hours, the mixture was cooled to room temperature and added potassium fluoride (2 g, 34.4 mmol) and stirred for 1 hour. The mixture was filtered and extracted with acetate (30 mL) for three times. The combined organic layers were washed with brine (30 mL), dried over Na₂SO₄₍ₛ₎ and filtered. The filtered was concentrated in vacuum reduced pressure to give the title compound (1.2 g, 48% purity from LCMS, 78.1% yield) as dark solids. LC-MS (ESI): R_{T}=1.36 min, mass calcd. for C₁₀H₁₂FNO₂ 197.1 m/z found 198.1 [M+H]⁺.

### Intermediate 45-5

### 5-Acetyl-3-fluoro-1-methylpyridin-2(1H)-one

To a solution of 5-(1-ethoxyvinyl)-3-fluoro-1-methylpyridin-2(1H)-one 45-4 (1.2 g, 48% purity, 2.92 mmol) in tetrahydrofuran (12 mL) was added 3 M hydrochloric acid (5 mL, 15 mmol) at 30° C. The resulting mixture was stirred at 30° C. for 2 hours. The reaction was adjusted to pH >7 with sodium bicarbonate solution. The mixture was concentrated in vacuum and filtered to get a residue, which was purified by silica gel column chromatography (petroleum ether:ethyl acetate=2:1) to give the title compound (650 mg, 70% purity from ¹H NMR, 92.1% yield) as yellow solids. LC-MS (ESI): R_{T}=0.42 min, mass calcd. for C₈H₈FNO₂ 169.0 m/z found 170.1 [M+H]⁺. ¹H NMR (300 MHz, CDCl₃) δ 7.99 (s, 1H), 7.31 (s, 1H), 3.73 (s, 3H), 2.50 (s, 3H).

### Intermediate 45-6

### Isopropyl (6R)-5-(3,4-dichlorobenzoyl)-2-((2R)-1-((1-(5-fluoro-1-methyl-6-oxo-1,6-dihydropyridin-3-yl)ethyl)amino)propan-2-yl)-6-methyl-4,5,6,7-tetrahydro-2H-pyrazolo[4,3-c]pyridine-3-carboxylate

To a solution of ethyl (R)-2-((R)-1-aminopropan-2-yl)-5-(3,4-dichlorobenzoyl)-6-methyl-4,5,6,7-tetrahydro-2H-pyrazolo[4,3-c]pyridine-3-carboxylate hydrochloride Int E (400 mg, 0.84 mmol) and 5-acetyl-3-fluoro-1-methylpyridin-2(1H)-one 45-6 (210 mg, 70% purity, 0.87 mmol) in tetrahydrofuran (5 mL) was added tetraisopropoxytitanium (4.8 g, 16.9 mmol). After stirred at 80° C. for 16 hours, sodium cyanoborohydride (150 mg, 2.39 mmol) and acetic acid (0.1 mL) were added at 0° C. and stirred for 2 hours. The reaction was diluted with brine (20 mL), filtered and extracted with acetate (10 mL) for three times. The combined organic layers were washed with brine (10 mL), dried over Na₂SO₄₍ₛ₎, filtered and concentrated under vacuum to give a residue, which was purified by C18 chromatography (acetonitrile:water (+0.02% ammonium bicarbonate)=45-60%) to give the title compound (240 mg, 72% purity from LCMS, 33.9% yield) as yellow solids. LC-MS (ESI): R_{T}=1.45 min, mass calcd. for C₂₉H₃₄Cl₂FN₅O₄ 605.2 m/z found 606.1 [M+H]⁺.

### Intermediate 45-7

### (6R)-5-(3,4-dichlorobenzoyl)-2-((2R)-1-((1-(5-fluoro-1-methyl-6-oxo-1,6-dihydro pyridin-3-yl)ethyl)amino)propan-2-yl)-6-methyl-4,5,6,7-tetrahydro-2H-pyrazolo [4,3-c]pyridine-3-carboxylic acid

To the solution of isopropyl (6R)-5-(3,4-dichlorobenzoyl)-2-((2R)-1-((1-(5-fluoro-1-methyl-6-oxo-1,6-dihydropyridin-3-yl)ethyl)amino)propan-2-yl)-6-methyl-4,5,6,7-tetrahydro-2H-pyrazolo[4,3-c]pyridine-3-carboxylate 45-6 (240 mg, 72% purity, 0.29 mmol) in methanol (3 mL) was added lithium hydroxide hydrate (50 mg, 1.19 mmol) at 0° C. After stirred at 30° C. for 2 hours, the mixture was diluted with water (5 mL), acidized with 0.5 M hydrochloride aqueous solution to pH˜5 and extracted with ethyl acetate (20 mL) twice. The combined organic layers were washed with brine (10 mL), dried over Na₂SO₄₍ₛ₎ and filtered. The filtrate was concentrated to give the title compound (190 mg, 83% purity from LCMS, 98.1% yield) as yellow solids. LC-MS (ESI): R_{T}=1.31 min, mass calcd. for C₂₆H₂₈Cl₂FN₅O₄ 563.1 m/z found 564.1 [M+H]⁺.

### Compound 45

### (3R,7R)-2-(3,4-dichlorobenzoyl)-9-(1-(5-fluoro-1-methyl-6-oxo-1,6-dihydro pyridin-3-yl)ethyl)-3,7-dimethyl-1,2,3,4,8,9-hexahydropyrido[4′,3′:3,4]pyrazolo [1,5-a]pyrazin-10(7H)-one

(6R)-5-(3,4-dichlorobenzoyl)-2-((2R)-1-((1-(5-fluoro-1-methyl-6-oxo-1,6-dihydropyridin-3-yl)ethyl)amino)propan-2-yl)-6-methyl-4,5,6,7-tetrahydro-2H-pyrazolo[4,3-c]pyridine-3-carboxylic acid 45-7 (190 mg, 83% purity, 0.28 mmol), 2-(3H-[1,2,3]triazolo[4,5-b]pyridin-3-yl)-1,1,3,3-tetramethylisouronium hexafluorophosphate(V) (180 mg, 0.47 mmol) were mixed in N,N-dimethylformamide (1.5 mL). After stirred at 0° C. for 10 min, triethylamine (100 mg, 0.99 mmol) in N,N-dimethylformamide (0.5 mL) was added dropwise to the reaction at 0° C. and stirring continued at 0° C. for 2 hours, the reaction mixture was quenched with brine (10 mL), extracted with acetate (10 mL) for three times, dried over Na₂SO₄₍ₛ₎ and filtered. The filtrate was concentrated to get a residue, which was purified by C18 chromatography (acetonitrile:water (+0.02% ammonium bicarbonate)=40-60%) to give the title compound (120 mg, 78% purity from LCMS, 61.3% yield) as white solids. LC-MS (ESI): R_{T}=1.53 min, mass calcd. for C₂₆H₂₆Cl₂N₅O₃ 545.1 m/z found 546.1 [M+H]⁺.

### Compounds 45A and 45B

### (3R,7R)-2-(3,4-dichlorobenzoyl)-9-((R*)-1-(5-fluoro-1-methyl-6-oxo-,6-dihydro pyridin-3-yl)ethyl)-3,7-dimethyl-1,2,3,4,8,9-hexahydropyrido[4′,3′:3,4]pyrazolo [1,5-a]pyrazin-10(7H)-one (45A) and (3R,7R)-2-(3,4-dichlorobenzoyl)-9-((S*)-1-(5-fluoro-1-methyl-6-oxo-1,6-dihydro pyridin-3-yl)ethyl)-3,7-dimethyl-1,2,3,4,8,9-hexahydropyrido[4′,3′:3,4]pyrazolo [1,5-a]pyrazin-10(7H)-one (45B)

The racemate of (3R,7R)-2-(3,4-dichlorobenzoyl)-9-(1-(5-fluoro-1-methyl-6-oxo-1,6-dihydropyridin-3-yl)ethyl)-3,7-dimethyl-1,2,3,4,8,9-hexahydropyrido[4′,3′:3,4]pyrazolo[1,5-a]pyrazin-10(7H)-one 45 (120 mg, 78% purity, 0.171 mmol) was separated by chiral prep HPLC (separation condition: Column: Chiralpak IE 5 μm 20*250 mm; Mobile Phase: IPA:ACN=30:70 at 25 mL/min; Temp: 30° C.; Wavelength: 254 nm) to give the title compounds 45A (35 mg, 99.2% purity, 37.1% yield, 100% stereopure) and 45B (25.3 mg, 99.8% purity, 27.0% yield, 99.9% stereopure) as white solids.

### 45A:

LC-MS (ESI): R_{T}=2.901 min, mass calcd. for C₂₆H₂₆Cl₂N₅O₃ 545.1 m/z found 546.2 [M+H]⁺. Chiral analysis (Column: Chiralpak IE 5 m 4.6*250 mm; Mobile Phase: ACN:IPA=70:30 at 1 mL/min; Temp: 30° C.; Wavelength: 254 nm, R_{T}=8.313 min). ¹H NMR (400 MHz, DMSO-d₆) δ 7.75 (d, J=8.0 Hz, 2H), 7.65-7.54 (m, 1H), 7.44 (dd, J=8.4 and 2.0 Hz, 1H), 7.42-7.30 (m, 1H), 5.68-5.11 (m, 2H), 4.63-4.34 (m, 2H), 4.26-4.04 (m, 1H), 3.77-3.64 (m, 1H), 3.52 (s, 3H), 3.23-3.11 (m, 1H), 2.98-2.86 (m, 1H), 2.67-2.51 (m, 1H), 1.51-1.35 (m, 3H), 1.31 (d, J=6.0 Hz, 3H), 1.23-1.07 (m, 3H). ¹⁹F NMR (376 MHz, DMSO-d₆) δ −132.96.

### 45B:

LC-MS (ESI): R_{T}=2.981 min, mass calcd. for C₂₆H₂₆Cl₂N₅O₃ 545.1 m/z found 546.2 [M+H]⁺. Chiral analysis (Column: Chiralpak IE 5 m 4.6*250 mm; Mobile Phase: ACN:IPA=70:30 at 1 mL/min; Temp: 30° C.; Wavelength: 254 nm, R_{T}=9.716 min). ¹H NMR (400 MHz, DMSO-d₆) δ 7.75 (d, J=8.0 Hz, 2H), 7.61-7.50 (m, 1H), 7.44 (d, J=8.4 Hz, 1H), 7.40-7.30 (m, 1H), 5.71-5.13 (m, 2H), 4.64-4.29 (m, 2H), 4.27-4.04 (m, 1H), 3.51 (s, 3H), 3.47-3.37 (m, 2H), 2.98-2.87 (m, 1H), 2.67-2.51 (m, 1H), 1.44-1.42 (m, 6H), 1.23-1.06 (m, 3H). ¹⁹F NMR (376 MHz, DMSO-d₆) δ −132.93.

### Compounds 46A and 46B

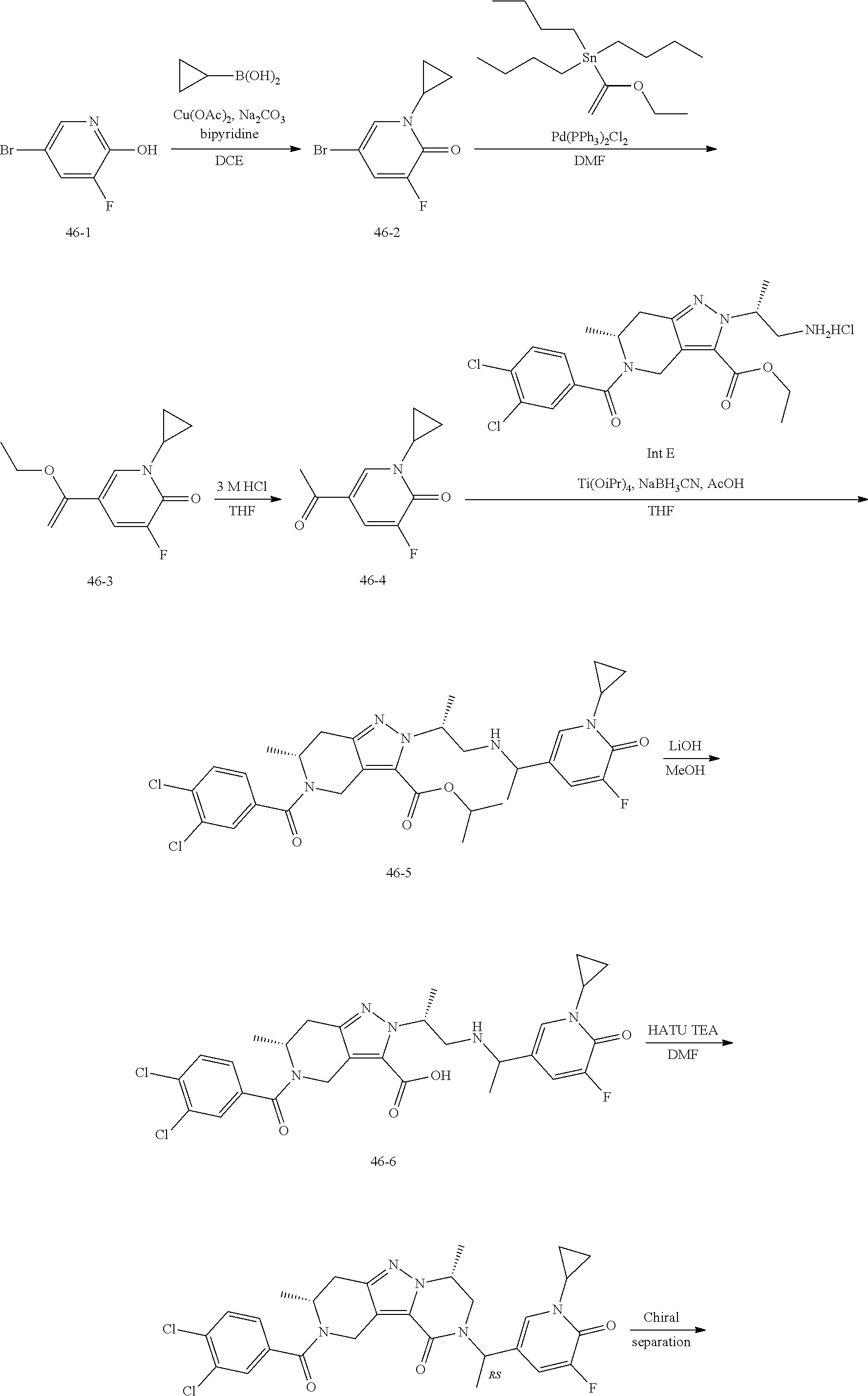

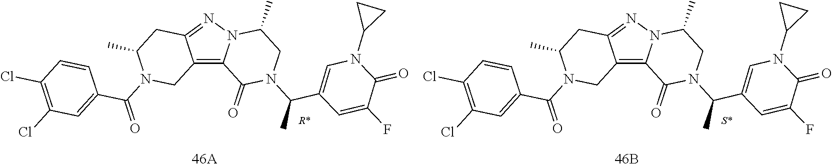

### Intermediate 46-2

### 5-Bromo-1-cyclopropyl-3-fluoropyridin-2(1H)-one

The mixture of 5-bromo-3-fluoropyridin-2-ol 46-1 (2.3 g, 60% purity, 7.19 mmol), cyclopropylboronic acid (1.3 g, 15.1 mmol), sodium carbonate (1.6 g, 15.1 mmol), copper (II) acetate (1.4 g, 7.71 mmol), 2,2′-bipyridine (1.3 g, 8.32 mmol) in 1,2-dichloroethane (80 mL) was stirred for 14 hours at 70° C. under nitrogen atmosphere. The solution was quenched with water (100 mL), extracted with dichloromethane (100 mL) for three times, dried over Na₂SO₄₍ₛ₎. The filtrate was concentrated under reduced pressure to get a residue, which was purified by silica gel column chromatography (petroleum ether:ethyl acetate=5:1) to give the title compound (700 mg, 78% purity from LCMS, 32.7% yield) as white solids. LC-MS (ESI): R_{T}=1.27 min, mass calcd. for C₈H₇BrNO 231.0 m/z found 231.9 [M+H]⁺.

### Intermediate 46-3

### 1-Cyclopropyl-5-(1-ethoxyvinyl)-3-fluoropyridin-2(1H)-one

To a solution of 5-bromo-1-cyclopropyl-3-fluoropyridin-2(1H)-one 46-2 (700 mg, 78% purity, 2.35 mmol) and tributyl(1-ethoxyethenyl)stannane (1.1 g, 3.05 mmol) in N,N-dimethylformamide (10 mL) was added bis(triphenylphosphine) palladium(II) dichloride (160 mg, 0.23 mmol) under nitrogen atmosphere. The resultant was stirred at 100° C. for 4 hours, then cooled to room temperature, potassium fluoride (900 mg, 15.5 mmol) was added and stirred for 1 hour. The mixture was filtered and extracted with acetate (30 mL) for three times. The combined organic layers were washed with brine (30 mL), dried over Na₂SO₄₍ₛ₎, filtered and concentrated in vacuum under reduced pressure to give the title compound (1.0 g, 50% purity from LCMS, 95.2% yield) as dark oil. LC-MS (ESI): R_{T}=1.46 min, mass calcd. for C₁₂H₁₄FNO₂ 223.1 m/z found 224.1 [M+H]⁺.

### Intermediate 46-4

### 5-Acetyl-1-cyclopropyl-3-fluoropyridin-2(1H)-one

To a solution of 1-cyclopropyl-5-(1-ethoxyvinyl)-3-fluoropyridin-2(1H)-one 46-3 (1.0 g, 50% purity, 2.24 mmol) in tetrahydrofuran (10 mL) was added 3 M hydrochloric acid solution (5 mL, 15 mmol) at 30° C. After stirred at 30° C. for 2 hours, the reaction was basified with sodium bicarbonate solution to pH >7. The mixture was concentrated in vacuum and filtered to get a residue, which was purified by silica gel column chromatography (petroleum ether:ethyl acetate=2:1) to give the title compound (550 mg, 59% purity from LCMS, 74.2% yield) as yellow oil. LC-MS (ESI): R_{T}=1.04 min, mass calcd. for C₁₀H₁₀FNO₂ 195.1 m/z found 196.1 [M+H]⁺. ¹H NMR (300 MHz, CDCl₃) δ 7.99 (s, 1H), 7.31 (s, 1H), 3.53-3.42 (m, 1H), 2.50 (s, 3H), 1.34-1.26 (m, 2H), 1.05-0.95 (m, 2H).

### Intermediate 46-5

### Isopropyl (6R)-2-((2R)-1-((1-(1-cyclopropyl-5-fluoro-6-oxo-1,6-dihydropyridin-3-yl)ethyl)amino)propan-2-yl)-5-(3,4-dichlorobenzoyl)-6-methyl-4,5,6,7-tetrahydro-2H-pyrazolo[4,3-c]pyridine-3-carboxylate

To a solution of ethyl (R)-2-((R)-1-aminopropan-2-yl)-5-(3,4-dichlorobenzoyl)-6-methyl-4,5,6,7-tetrahydro-2H-pyrazolo[4,3-c]pyridine-3-carboxylate hydrochloride Int E (200 mg, 100% purity, 0.42 mmol) and 5-acetyl-1-cyclopropyl-3-fluoropyridin-2(1H)-one 46-4 (180 mg, 50% purity, 0.46 mmol) in tetrahydrofuran (3 mL) were added tetraisopropoxytitanium (2.4 g, 8.44 mmol) and acetic acid (0.1 mL) at 30° C., then the mixture was stirred at 80° C. for 16 hours. Sodium cyanoborohydride (80 mg, 1.27 mmol) was added into the mixture and stirred at 0° C. for 2 hours. The mixture was diluted with brine (20 mL), filtered and extracted with acetate (10 mL) for three times. The combined organic layers were washed with brine (10 mL), dried over Na₂SO₄₍ₛ₎, filtered and concentrated under vacuum to give a residue, which was purified by C18 chromatography (acetonitrile:water (+0.02% ammonium bicarbonate)=45-60%) to give the title product compound (150 mg, 86% purity from LCMS, 48.5% yield) as yellow solids. LC-MS (ESI): R_{T}=1.72 min, mass calcd. for C₃₁H₃₆Cl₂FN₅O₄ 631.2 m/z found 632.2 [M+H]⁺.

### Intermediate 46-6

### (6R)-2-((2R)-1-((1-(1-cyclopropyl-5-fluoro-6-oxo-1,6-dihydropyridin-3-yl)ethyl) amino)propan-2-yl)-5-(3,4-dichlorobenzoyl)-6-methyl-4,5,6,7-tetrahydro-2H-pyrazolo[4,3-c]pyridine-3-carboxylic acid

To the solution of isopropyl (6R)-2-((2R)-1-((1-(1-cyclopropyl-5-fluoro-6-oxo-1,6-dihydropyridin-3-yl)ethyl)amino)propan-2-yl)-5-(3,4-dichlorobenzoyl)-6-methyl-4,5,6,7-tetrahydro-2H-pyrazolo[4,3-c]pyridine-3-carboxylate 46-5 (150 mg, 86% purity, 0.20 mmol) in methanol (1 mL) was added lithium hydroxide hydrate (30 mg, 0.72 mmol) at 0° C. After stirred at 30° C. for 2 hours, the mixture was diluted with water (5 mL), acidized with 0.5 M hydrochloric acid aqueous solution to pH˜5 and extracted with ethyl acetate (10 mL) twice. The combined organic layers were washed with brine (10 mL), dried over Na₂SO₄₍ₛ₎ and filtered. The filtrate was concentrated to give the crude compound (150 mg, 70.9% purity from LCMS, 88.3% yield) as yellow solids. LC-MS (ESI): R_{T}=1.34 min and 1.58 min, mass calcd. for C₂₈H₃₀Cl₂FN₅O₄ 589.2 m/z found 590.1 [M+H]⁺.

### Compound 46

### (3R,7R)-9-(1-(1-cyclopropyl-5-fluoro-6-oxo-1,6-dihydropyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

(6R)-2-((2R)-1-((1-(1-cyclopropyl-5-fluoro-6-oxo-1,6-dihydropyridin-3-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid 46-6 (150 mg, 70.9% purity, 0.18 mmol), 2-(3H-[1,2,3]triazolo[4,5-b]pyridin-3-yl)-1,1,3,3-tetramethylisouronium hexafluorophosphate(V) (110 mg, 0.29 mmol) were mixed in N,N-dimethylformamide (1.5 mL). After stirred at 0° C. for 10 minutes, triethylamine (70 mg, 0.69 mmol) in N,N-dimethylformamide (0.5 mL) was added dropwise at 0° C. After stirred at 0° C. for 2 hours, the reaction mixture was quenched with brine (10 mL) and extracted with acetate (10 mL) for three times, dried over Na₂SO₄₍ₛ₎ and filtered. The filtrate was concentrated to get a residue, which was purified by C18 chromatography (acetonitrile:water (+0.02% ammonium bicarbonate)=40-60%) to give the title compound (70 mg, 100% purity from LCMS, 67.9% yield) as white solids. LC-MS (ESI): R_{T}=2.41 min, mass calcd. for C₂₈H₂₈Cl₂FN₅O₃ 571.2 m/z found 572.1 [M+H]⁺.

### Compounds 46A and 46B

### (3R,7R)-9-((R*)-1-(1-cyclopropyl-5-fluoro-6-oxo-1,6-dihydropyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (46A) and (3R,7R)-9-((S*)-1-(1-cyclopropyl-5-fluoro-6-oxo-1,6-dihydropyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (46B)

The racemate of (3R,7R)-9-(1-(1-cyclopropyl-5-fluoro-6-oxo-1,6-dihydropyridin-3-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 46 (120 mg, 98% purity, 0.21 mmol) was separated by chiral prep HPLC (separation condition: Column: Chiralpak IE 5 μm 20*250 mm; Mobile Phase: IPA:ACN=50:50 at 25 mL/min; Temp: 30° C.; Wavelength: 254 nm) to give the title compounds 46A (34.0 mg, 99.2% purity, 28.7% yield, 100% stereopure) and 46B (23.5 mg, 98.8% purity, 19.7% yield, 99.9% stereopure) as white solids.

### 46A:

LC-MS (ESI): R_{T}=3.787 min, mass calcd. for C₂₈H₂₈Cl₂FN₅O₃ 571.2 m/z found 572.3 [M+H]⁺. Chiral analysis (Column: Chiralpak IE 5 μm 4.6*250 mm; Mobile Phase: ACN:IPA=50:50 at 1 mL/min; Temp: 30° C.; Wavelength: 254 nm, R_{T}=6.816 min). ¹H NMR (400 MHz, DMSO-d₆) δ 7.75-7.73 (m, 2H), 7.44 (dd, J=8.4, 2.0 Hz, 1H), 7.42-7.18 (m, 2H), 5.64-5.14 (m, 2H), 4.62-4.32 (m, 2H), 4.24-4.05 (m, 1H), 3.81-3.65 (m, 1H), 3.25-3.07 (m, 2H), 2.98-2.86 (m, 1H), 2.65-2.51 (m, 1H), 1.55-1.37 (m, 3H), 1.31 (d, J=6.8 Hz, 3H), 1.24-1.08 (m, 3H), 1.04-1.02 (m, 2H), 0.97-0.84 (m, 2H). ¹⁹F NMR (376 MHz, DMSO-d₆) δ −132.87.

### 46B:

LC-MS (ESI): R_{T}=3.826 min, mass calcd. for C₂₈H₂₈Cl₂FN₅O₃ 571.2 m/z found 572.3 [M+H]⁺. Chiral analysis (Column: Chiralpak IE 5 μm 4.6*250 mm; Mobile Phase: ACN:IPA=50:50 at 1 mL/min; Temp: 30° C.; Wavelength: 254 nm, R_{T}=7.703 min). ¹H NMR (400 MHz, DMSO-d₆) 6.75 (d, J=8.0 Hz, 2H), 7.44 (d, J=8.8 Hz, 1H), 7.37-7.17 (m, 2H), 5.65-5.15 (m, 2H), 4.62-4.27 (m, 2H), 4.24-4.02 (m, 1H), 3.52-3.37 (m, 2H), 3.31-3.27 (m, 1H), 2.96-2.87 (m, 1H), 2.66-2.51 (m, 1H), 1.49-1.36 (m, 6H), 1.25-1.09 (m, 3H), 1.06-0.98 (m, 2H), 0.96-0.86 (m, 2H). ¹⁹F NMR (376 MHz, DMSO-d₆) δ −132.93.

### Compounds 47A, 47B, 48A and 48B

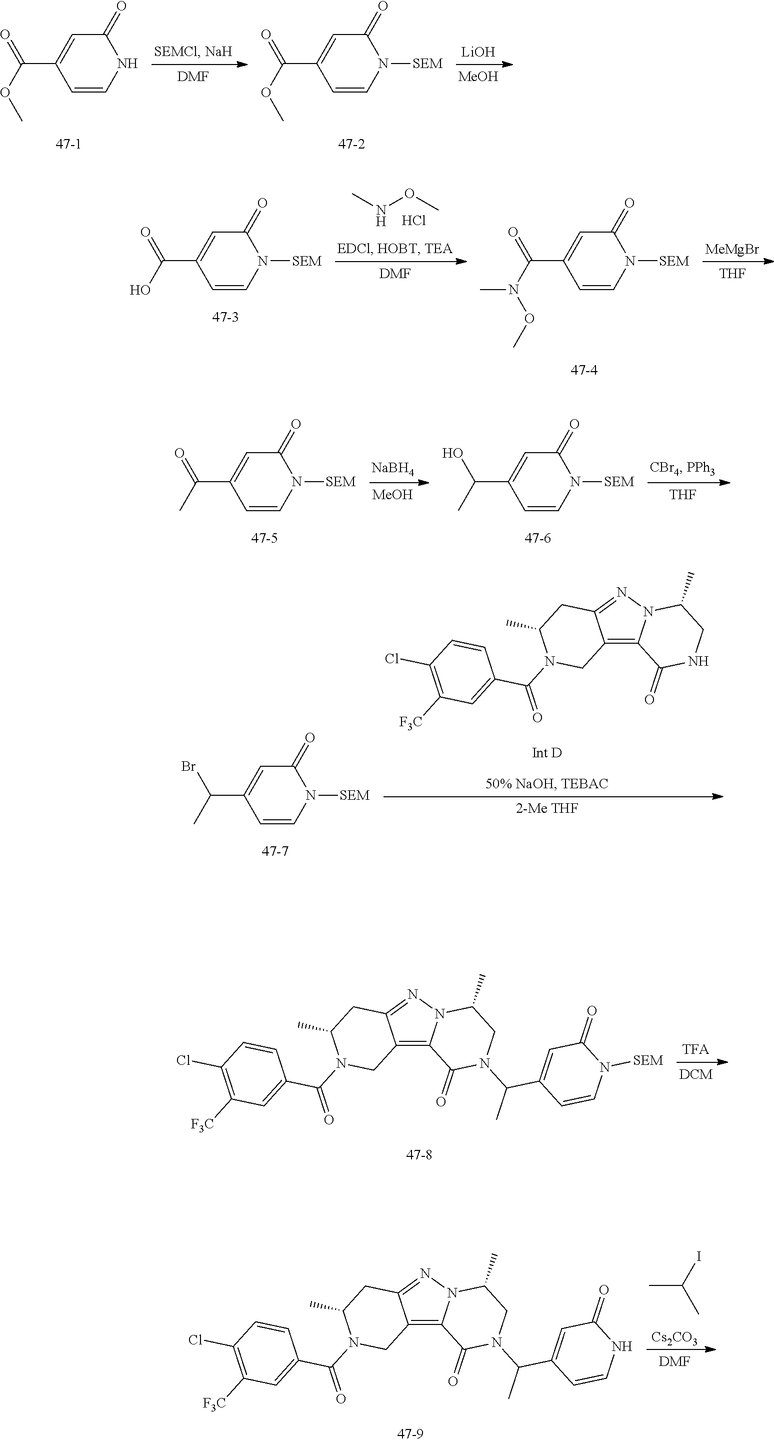

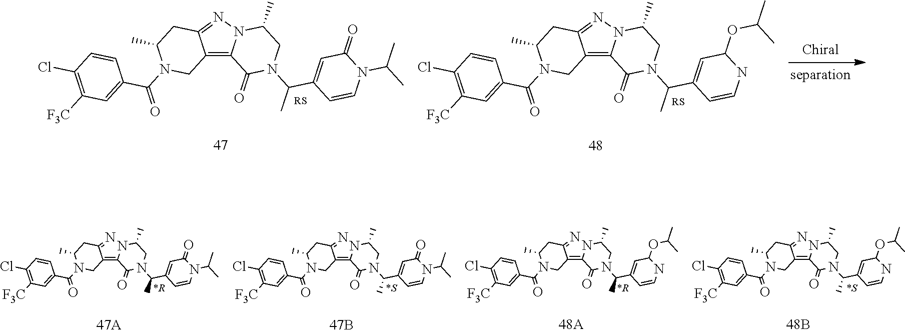

### Intermediate 47-2

### Methyl 2-oxo-1-((2-(trimethylsilyl)ethoxy)methyl)-1,2-dihydropyridine-4-carboxylate

To a solution of methyl 2-oxo-1,2-dihydropyridine-4-carboxylate 47-1 (3.0 g, 19.59 mmol) in N,N-dimethylformamide (40 mL) was added sodium hydride 60% wt. (1.0 g, 25.0 mmol) at 0° C. After stirred at 0° C. for 1 hour, (2-(chloromethoxy)ethyl)trimethylsilane (4.5 mL, 25.43 mmol) was added and the mixture was stirred at room temperature for 4.5 hours. Then the mixture was poured into ice water (50 mL), and extracted with ethyl acetate (100 mL) for three times. The combined organic layers were washed with brine (30 mL), dried over Na₂SO₄₍ₛ₎ and filtered. The filtrate was concentrated and purified by column chromatography on silica gel (petroleum ether:ethyl acetate=70:1 to 20:1) to give the compound (2.23 g, 70% purity from LCMS, 28% yield) as yellow oil. LC-MS (ESI): R_{T}=1.511 min, mass calcd. for C₁₃H₂₁NO₄Si 283.1, m/z found 284.1 [M+H]⁺.

### Intermediate 47-3

### 2-oxo-1-((2-(Trimethylsilyl)ethoxy)methyl)-1,2-dihydropyridine-4-carboxylic acid

To a mixture of methyl 2-oxo-1-((2-(trimethylsilyl)ethoxy)methyl)-1,2-dihydropyridine-4-carboxylate 47-2 (2.23 g, 70% purity, 5.51 mmol) in methanol (20 mL) was added lithium hydroxide (350 mg, 8.34 mmol) at 0° C. The reaction was allowed to slowly return to room temperature. After stirred at room temperature for 4.5 hours, the reaction mixture was acidified with 0.5 M hydrochloride aqueous solution to pH 5-6, extracted with ethyl acetate (50 mL) for three times. The combined organic layers were washed with brine (20 mL), dried over Na₂SO₄₍ₛ₎, filtered. The filtrate was concentrated to give the title compound (1.73 g, 84% purity from LCMS, 98% yield) as white solids. LC-MS (ESI): R_{T}=1.033 min, mass calcd. for Cl₂H₁₉NO₄Si 269.1, m/z found 270.1 [M+H]⁺.

### Intermediate 47-4

### N-Methoxy-N-methyl-2-oxo-1-((2-(trimethylsilyl)ethoxy)methyl)-1,2-dihydropyridine-4-carboxamide

To a solution of 2-oxo-1-((2-(trimethylsilyl)ethoxy)methyl)-1,2-dihydropyridine-4-carboxylic acid 47-3 (1.73 g, 84% purity, 5.40 mmol), N,O-dimethylhydroxylamine hydrochloride (1.3 g, 13.33 mmol), N-(3-dimethylaminopropyl)-N′-ethyl carbodiimide hydrochloride (2.1 g, 10.96 mmol) and 1H-benzo[d][1,2,3]triazol-1-ol (1.5 g, 11.10 mmol) in N,N-dimethylformamide (30 mL) was slowly added triethylamine (4.5 mL, 32.46 mmol) at 0° C. The reaction was allowed to slowly return to room temperature. After stirred at room temperature under nitrogen atmosphere for 3 hours, the reaction mixture was quenched with water (30 mL) and extracted with ethyl acetate (50 mL) for three times. The combined organic layers were dried over Na₂SO₄₍ₛ₎ and filtered. The filtrate was concentrated and purified by C18 column (acetonitrile: water=5% to 100%) to give the title compound (1.65 g, 100% purity from LCMS, 98% yield) as colorless oil. LC-MS (ESI): R_{T}=1.418 min, mass calcd. for C₁₄H₂₄N₂O₄Si 312.1, m/z found 313.2 [M+H]⁺.

### Intermediate 47-5

### 4-Acetyl-1-((2-(trimethylsilyl)ethoxy)methyl)pyridin-2(1H)-one

To the solution of N-methoxy-N-methyl-2-oxo-1-((2-(trimethylsilyl)ethoxy)methyl)-1,2-dihydropyridine-4-carboxamide 47-4 (1.65 g, 100% purity, 5.28 mmol) in dry tetrahydrofuran (20 mL) was added 1 M methylmagnesium bromide in tetrahydrofuran (8 mL, 8 mmol) at 0° C. The mixture was stirred at 0° C. for 1.5 hours. The mixture was quenched with ammonium chloride aqueous solution (15 mL) and extracted with ethyl acetate (20 mL) twice. The combined organic layers were washed with brine (10 mL), dried over Na₂SO₄₍ₛ₎ and filtered. The filtrate was concentrated and purified by silica gel column chromatography (petroleum ether:acetone=50:1 to 20:1) to give the title compound (1.37 g, 97% purity from LCMS, 94% yield) as yellow oil. LC-MS (ESI): R_{T}=1.356 min, mass calcd. for Cl₃H₂₁NO₃Si 267.1, m/z found 268.1 [M+H]⁺.

### Intermediate 47-6

### 4-(1-Hydroxyethyl)-1-((2-(trimethylsilyl)ethoxy)methyl)pyridin-2(1H)-one

To a solution of 4-acetyl-1-((2-(trimethylsilyl)ethoxy)methyl)pyridin-2(1H)-one 47-5 (1.37 g, 97% purity, 4.97 mmol) in methanol (12 mL) was slowly added sodium tetrahydroborate (180 mg, 4.76 mmol) at 0° C. After stirred at 0° C. for 2 hours, the reaction was quenched with acetone (10 mL) dropwise and concentrated to give the title compound (1.4 g, 95% purity from LCMS, 99% yield) as yellow oil. LC-MS (ESI): R_{T}=1.225 min, mass calcd. for Cl₃H₂₃NO₃Si 269.1, m/z found 270.1 [M+H]⁺.

### Intermediate 47-7

### 4-(1-Bromoethyl)-1-((2-(trimethylsilyl)ethoxy)methyl)pyridin-2(1H)-one

To a solution of 4-(1-hydroxyethyl)-1-((2-(trimethylsilyl)ethoxy)methyl)pyridin-2(1H)-one 47-6 (1.26 g, 95% purity, 4.44 mmol) in tetrahydrofuran (20 mL) were added triphenylphosphine (2.05 g, 7.82 mmol) and perbromomethane (2.05 g, 6.18 mmol) at 0° C. After stirred at room temperature for 2.5 hours, the mixture was filtered. The filtrate was concentrated and purified by column chromatography on silica gel (petroleum ether:acetone=50:1) to give the title compound (1.22 g, 99% purity from LCMS, 81% yield) as colorless oil. LC-MS (ESI): R_{T}=1.489 min, mass calcd. for Cl₃H₂₂BrNO₂Si 331.1, m/z found 332.0 [M+H]⁺.

### Intermediate 47-8

### (3R,7R)-2-(4-chloro-3-(trifluoromethyl)benzoyl)-3,7-dimethyl-9-(1-(2-oxo-1-((2-(trimethylsilyl)ethoxy)methyl)-1,2-dihydropyridin-4-yl)ethyl)-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-1,2,3,4,8,9-hexahydropyrido[4′,3′:3,4]pyrazolo[1,5-a]pyrazin-10(7H)-one Int D (2.10 g, 90% purity, 4.43 mmol), N-benzyl-N,N-diethylethanaminium chloride (205 mg, 0.90 mmol) and 4-(1-bromoethyl)-1-((2-(trimethylsilyl)ethoxy)methyl)pyridin-2(1H)-one 47-7 (2.00 g, 90% purity, 5.42 mmol) in 2-methyltetrahydrofuran (20 mL) was added 50% wt. sodium hydroxide in water (20 mL) slowly at 0° C. After stirred at room temperature for 3 hours, the mixture was quenched with water (50 mL) slowly and extracted with ethyl acetate (50 mL) for three times. The separated organic layer was washed with brine (50 mL), dried over Na₂SO₄₍ₛ₎ and filtered. The filtrate was concentrated under reduced pressure to give a residue, which was purified by C18 column (acetonitrile:water (+0.1% ammonium bicarbonate)=30% to 95%) to give the title compound (2.50 g, 90% purity from LCMS, 75% yield) as white solids. LC-MS (ESI): R_{T}=1.80 min, mass calcd. for C₃₂H₃₉ClF₃N₅O₄Si 677.2, m/z found 678.1 [M+H]⁺.

### Intermediate 47-9

### (3R,7R)-2-(4-chloro-3-(trifluoromethyl)benzoyl)-3,7-dimethyl-9-(1-(2-oxo-1,2-dihydropyridin-4-yl)ethyl)-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-9-(1-(2-oxo-1-((2-(trimethylsilyl)ethoxy)methyl)-1,2-dihydropyridin-4-yl)ethyl)-1,2,3,4,8,9-hexahydropyrido[4′,3′:3,4]pyrazolo[1,5-a]pyrazin-10(7H)-one 47-8 (2.50 g, 90% purity, 3.32 mmol) in dichloromethane (15 mL) was added 2,2,2-trifluoroacetic acid (15 mL) at 0° C. After stirred at room temperature for 1 hour under nitrogen atmosphere, the reaction mixture was quenched with saturated sodium bicarbonate aqueous solution (50 mL) at 0° C. and extracted with dichloromethane (50 mL) for three times. The separated organic layer was washed with brine (50 mL), dried over Na₂SO₄₍ₛ₎, filtered and concentrated under reduced pressure to give the title compound (1.90 g, 90% purity from LCMS, 94% yield) as yellow solids. LC-MS (ESI): R_{T}=1.46 min, mass calcd. for C₂₆H₂₅ClF₃N₅O₃ 547.2, m/z found 548.1 [M+H]⁺.

### Compounds 47 and 48

### (3R,7R)-2-(4-chloro-3-(trifluoromethyl)benzoyl)-9-(1-(1-isopropyl-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (47) and (3R,7R)-2-(4-chloro-3-(trifluoromethyl)benzoyl)-9-(1-(2-isopropoxypyridin-4-yl)ethyl)-3,7-dimethyl-1,2,3,4,8,9-hexahydropyrido[4′,3′:3,4]pyrazolo[1,5-a]pyrazin-10(7H)-one (48)

To a solution of (3R,7R)-2-(4-chloro-3-(trifluoromethyl)benzoyl)-3,7-dimethyl-9-(1-(2-oxo-1,2-dihydropyridin-4-yl)ethyl)-1,2,3,4,8,9-hexahydropyrido[4′,3′:3,4]pyrazolo [1,5-a]pyrazin-10(7H)-one 47-9 (1.00 g, 90% purity, 1.64 mmol) in N,N-dimethylformamide (2 mL) were added 2-iodopropane (0.5 mL, 5.01 mmol) and cesium carbonate (1.60 g, 4.91 mmol). After heated at 80° C. for 12 hours, the reaction mixture was washed with water (100 mL) and extracted with ethyl acetate (100 mL) for three times. The combined organic layers were washed with brine (100 mL), dried over anhydrous sodium sulfate, filtered and concentrated. The residue was purified by C18 column (acetonitrile:water (+0.1% ammonium bicarbonate)=45% to 75%) to give the title compounds 47 (220 mg, 90% purity form LCMS, 21% yield) as yellow solids and 48 (350 mg, 99% purity form LCMS, 36% yield) as yellow solids. 47: LC-MS (ESI): R_{T}=1.60 min, mass calcd. for C₂₉H₃₁ClF₃N₅O₃ 589.2, m/z found 590.2 [M+H]⁺. 48: LC-MS (ESI): R_{T}=4.40 min, mass calcd. for C₂₉H₃₁ClF₃N₅O₃ 589.2, m/z found 590.4 [M+H]⁺.

### Compounds 47A and 47B

### (3R,7R)-2-(4-chloro-3-(trifluoromethyl)benzoyl)-9-((R*)-1-(1-isopropyl-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (47A) and (3R,7R)-2-(4-chloro-3-(trifluoromethyl)benzoyl)-9-((S*)-1-(1-isopropyl-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (47B)

(3R,7R)-2-(4-Chloro-3-(trifluoromethyl)benzoyl)-9-(1-(1-isopropyl-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one 47 (220 mg, 90% purity, 0.34 mmol) was separated by chiral Prep. HPLC (separation condition: Column: Chiralpak IE 10 μm 30*250 mm; Mobile Phase: acetonitrile:isopropanol=50:50 at 25 mL/min, Temp: 30° C.; Wavelength: 214 nm) to give the title compounds 47A (35 mg, 99.1% purity, 18% yield, 100% stereopure) as white solids and 47B (47 mg, 99.5% purity, 24% yield, 99.9% stereopure) as white solids.

### 47A:

LC-MS (ESI): R_{T}=10.304 min, mass calcd. for C₂₉H₃₁ClF₃N₅O₃ 589.2, m/z found 590.0 [M+H]⁺. Chiral HPLC (Column: Chiralpak IE 5 μm 4.6*250 mm; Mobile Phase: ACN:IPA=50:50 at 1 mL/min; Temp: 30° C.; Wavelength: 254 nm, R_{T}=6.217 min). 1H NMR (400 MHz, CDCl₃) δ: 7.85-7.73 (m, 1H), 7.63-7.50 (m, 2H), 7.31-7.28 (m, 1H), 6.58-6.48 (m, 1H), 6.25-6.11 (m, 1H), 5.91-5.70 (m, 1H), 5.50-5.15 (m, 2H), 4.86-4.32 (m, 3H), 3.72-3.51 (m, 1H), 3.22-2.90 (m, 2H), 2.76-2.58 (m, 1H), 1.51-1.45 (m, 3H), 1.43-1.38 (m, 3H), 1.37-1.25 (m, 9H). ¹⁹F NMR (376 MHz, CDCl₃) δ −62.81.

### 47B:

LC-MS (ESI): R_{T}=10.299 min, mass calcd. for C₂₉H₃₁ClF₃N₅O₃ 589.2, m/z found 590.1 [M+H]⁺. Chiral HPLC (Column: Chiralpak IE 5 μm 4.6*250 mm; Mobile Phase: ACN:IPA=50:50 at 1 mL/min; Temp: 30° C.; Wavelength: 254 nm, R_{T}=9.186 min). ¹H NMR (400 MHz, CDCl₃) δ 7.83-7.76 (m, 1H), 7.63-7.51 (m, 2H), 7.25 (s, 1H), 6.56-6.47 (m, 1H), 6.25-6.02 (m, 1H), 5.92-5.65 (m, 1H), 5.55-5.14 (m, 2H), 4.95-4.25 (m, 3H), 3.45-3.22 (m, 2H), 3.17-2.57 (m, 2H), 1.57 (d, J=6.6 Hz, 3H), 1.52-1.44 (m, 3H), 1.24-1.40 (m, 9H). ¹⁹F NMR (376 MHz, CDCl₃) δ −62.79.

### Compounds 48A and 48B

### (3R,7R)-2-(4-chloro-3-(trifluoromethyl)benzoyl)-9-((R*)-1-(2-isopropoxypyridin-4-yl)ethyl)-3,7-dimethyl-1,2,3,4,8,9-hexahydropyrido[4′,3′:3,4]pyrazolo [1,5-a]pyrazin-10(7H)-one (48A) and (3R,7R)-2-(4-chloro-3-(trifluoromethyl)benzoyl)-9-((S*)-1-(2-isopropoxypyridin-4-yl)ethyl)-3,7-dimethyl-1,2,3,4,8,9-hexahydropyrido[4′,3′:3,4]pyrazolo[1,5-a]pyrazin-10(7H)-one (48B)

(3R,7R)-2-(4-Chloro-3-(trifluoromethyl)benzoyl)-9-(1-(2-isopropoxypyridin-4-yl)ethyl)-3,7-dimethyl-1,2,3,4,8,9-hexahydropyrido[4′,3′:3,4]pyrazolo[1,5-a]pyrazin-10(7H)-one 48 (350 mg, 90% purity, 0.587 mmol) was separated by chiral Prep. HPLC (separation condition: Column: Chiralpak IG 5 μm 30*250 mm; Mobile Phase: acetonitrile:isopropanol=90:10 at 25 mL/min, Temp: 30° C.; Wavelength: 214 nm) to give the title compounds 48A (62 mg, 98.8% purity, 18% yield, 99.8% stereopure) as white solids and 48B (97 mg, 99.7% purity, 28% yield, 99.7% stereopure) as white solids.

### 48A:

LC-MS (ESI): R_{T}=9.985 min, mass calcd. for C₂₉H₃₁ClF₃N₅O₃ 589.2, m/z found 590.2 [M+H]⁺. Chiral HPLC (Column: Chiralpak IE 5 μm 4.6*250 mm; Mobile Phase: ACN:IPA=90:10 at 1 mL/min; Temp: 30° C.; Wavelength: 254 nm, R_{T}=5.326 min). ¹H NMR (400 MHz, CDCl₃) δ 8.22-7.94 (m, 1H), 7.82-7.65 (m, 1H), 7.61-7.37 (m, 2H), 7.23-7.13 (m, 1H), 6.84-6.52 (m, 2H), 6.04-5.78 (m, 1H), 5.60-5.15 (m, 2H), 4.87-4.46 (m, 1H), 4.42-4.24 (m, 1H), 3.69-3.43 (m, 1H), 3.20-2.86 (m, 2H), 2.77-2.51 (m, 1H), 1.58-1.42 (m, 6H), 1.38-1.27 (m, 9H). ¹⁹F NMR (376 MHz, CDCl₃) δ −62.79.

### 48B:

LC-MS (ESI): R_{T}=9.947 min, mass calcd. for C₂₉H₃₁ClF₃N₅O₃ 589.2, m/z found 590.2 [M+H]⁺. Chiral HPLC (Column: Chiralpak IE 5 μm 4.6*250 mm; Mobile Phase: ACN:IPA=90:10 at 1 mL/min; Temp: 30° C.; Wavelength: 254 nm, R_{T}=7.073 min). ¹H NMR (400 MHz, CDCl₃) δ 8.19-7.99 (m, 1H), 7.79 (s, 1H), 7.64-7.49 (m, 2H), 7.25 (s, 1H), 6.89-6.45 (m, 2H), 6.08-5.81 (m, 1H), 5.59-5.20 (m, 2H), 4.96-4.44 (m, 1H), 4.39-4.23 (m, 1H), 3.40-2.92 (m, 3H), 2.80-2.53 (m, 1H), 1.55 (d, J=6.6 Hz, 6H), 1.38-1.21 (m, 9H). ¹⁹F NMR (376 MHz, CDCl₃) δ −62.80.

### Compounds 49A, 49B, 50A and 50B

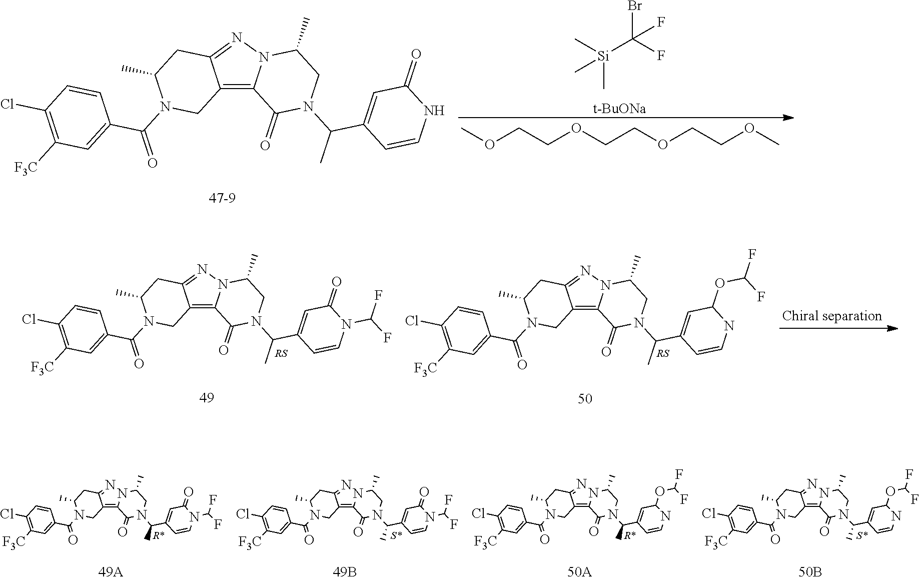

Intermediates 49 and 50

### (3R,7R)-2-(4-chloro-3-(trifluoromethyl)benzoyl)-9-(1-(1-(difluoromethyl)-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (49) and (3R,7R)-2-(4-chloro-3-(trifluoromethyl)benzoyl)-9-(1-(2-(difluoromethoxy)pyridin-4-yl)ethyl)-3,7-dimethyl-1,2,3,4,8,9-hexahydropyrido[4′,3′:3,4]pyrazolo[1,5-a]pyrazin-10(7H)-one (50)

To a mixture of (3R,7R)-2-(4-chloro-3-(trifluoromethyl)benzoyl)-3,7-dimethyl-9-(1-(2-oxo-1,2-dihydropyridin-4-yl)ethyl)-1,2,3,4,8,9-hexahydropyrido[4′,3′:3,4]pyrazolo[1,5-a]pyrazin-10(7H)-one 47-9 (650 mg, 90% purity, 1.07 mmol) and sodium 2-methylpropan-2-olate (226 mg, 2.35 mmol) was added 2,5,8,11-tetraoxadodecane (7 mL) at −15° C. under nitrogen atmosphere. After stirred at −15° C. for 15 minutes, a solution of (bromodifluoromethyl)trimethylsilane (239 mg, 1.18 mmol) in 2,5,8,11-tetraoxadodecane (6 mL) was added slowly. After stirred at −15° C. for 1 hour, the reaction mixture was quenched with water (20 mL) at 0° C. and extracted with ethyl acetate (20 mL) for three times. The separated organic layer was washed with brine (20 mL), dried over Na₂SO₄₍ₛ₎, filtered and concentrated under reduced pressure to give a residue, which was purified by C18 column (acetonitrile:water (+0.1% ammonium bicarbonate)=5% to 95%) to give the title compounds 49 (200 mg, 90% purity from ¹H NMR, 28% yield) as white solids and 50 (240 mg, 86% purity from LCMS, 32% yield) as white solids.

49:

LC-MS (ESI): R_{T}=1.204 min, mass calcd. for C₂₇H₂₅ClF₅N₅O₃ 597.2, m/z found 598.0 [M+H]⁺.

50:

LC-MS (ESI): R_{T}=1.76 min, mass calcd. for C₂₇H₂₅ClF₅N₅O₃ 597.2, m/z found 598.1 [M+H]⁺.

### Compounds 49A and 49B

### (3R,7R)-2-(4-chloro-3-(trifluoromethyl)benzoyl)-9-((R*)-1-(1-(difluoromethyl)-2-oxo-1,2-dihydropyridin-4-yl)ethyl)-3,7-dimethyl-1,2,3,4,8,9-hexahydropyrido [4′,3′:3,4]pyrazolo[1,5-a]pyrazin-10(7H)-one (49A) and (3R,7R)-2-(4-chloro-3-(trifluoromethyl)benzoyl)-9-((S*)-1-(1-(difluoromethyl)-2-oxo-1,2-dihydropyridin-4-yl)ethyl)-3,7-dimethyl-1,2,3,4,8,9-hexahydropyrido [4′,3′:3,4]pyrazolo[1,5-a]pyrazin-10(7H)-one (49B)

A racemic mixture of (3R,7R)-2-(4-chloro-3-(trifluoromethyl)benzoyl)-9-(1-(1-(difluoromethyl)-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one 49 (200 mg, 90% purity, 0.301 mmol) was separated by Chiral Prep-HPLC (Column: Chiralpak IE 5 μm 30 mm*250 mm; Mobile Phase: ACN:IPA=80:20 at 25 mL/min; Col. Temp: 30° C.; Wavelength: 230 nm) to afford the title compounds 49A (53 mg, 98.7% purity from LCMS, 29% yield, 99.9% stereopure) as white solids and 49B (55 mg, 99.3% purity from LCMS, 30% yield, 99.8% stereopure) as white solids.

### 49A:

LC-MS (ESI): R_{T}=3.502 min, mass calcd. for C₂₇H₂₅ClF₅N₅O₃ 597.2, m/z found 598.2 [M+H]⁺. Chiral analysis (Method: Column: Chiralpak IE 5 μm 4.6*250 mm; Mobile Phase: ACN:IPA=80:20 at 1 mL/min; Temp: 30° C.; Wavelength: 254 nm; SFC: R_{T}=5.490 min). ¹H NMR (400 MHz, DMSO-d₆) δ 7.98-7.68 (m, 5H), 6.48 (s, 1H), 6.38-6.22 (m, 1H), 5.62-5.18 (m, 2H), 4.59-4.39 (m, 2H), 4.21-4.08 (m, 1H), 3.84-3.69 (m, 1H), 3.27-3.15 (m, 1H), 2.97-2.92 (m, 1H), 2.66-2.58 (m, 1H), 1.54-1.42 (m, 3H), 1.35 (d, J=6.8 Hz, 3H), 1.23-1.09 (m, 3H). ¹⁹F NMR (376 MHz, DMSO-d₆) δ −61.33, −103.17.

### 49B:

LC-MS (ESI): R_{T}=3.533 min, mass calcd. for C₂₇H₂₅ClF₅N₅O₃ 597.2, m/z found 598.2 [M+H]⁺. Chiral analysis (Method: Column: Chiralpak IE 5 μm 4.6*250 mm; Mobile Phase: ACN:IPA=80:20 at 1 mL/min; Temp: 30° C.; Wavelength: 254 nm; SFC: R_{T}=6.774 min). ¹H NMR (400 MHz, DMSO-d₆) δ 7.98-7.68 (m, 5H), 6.50-6.41 (m, 1H), 6.37-6.23 (m, 1H), 5.66-5.19 (m, 2H), 4.62-4.35 (m, 2H), 4.23-4.06 (m, 1H), 3.58-3.42 (m, 2H), 2.97-2.92 (m, 1H), 2.66-2.58 (m, 1H), 1.53-1.40 (m, 6H), 1.21-1.08 (m, 3H). ¹⁹F NMR (376 MHz, DMSO-d₆) δ −61.34, −103.15.

### Compounds 50A and 50B

### (3R,7R)-2-(4-chloro-3-(trifluoromethyl)benzoyl)-9-((R*)-1-(2-(difluoromethoxy) pyridin-4-yl)ethyl)-3,7-dimethyl-1,2,3,4,8,9-hexahydropyrido[4′,3′:3,4]pyrazolo[1,5-a]pyrazin-10(7H)-one (50A) and (3R,7R)-2-(4-chloro-3-(trifluoromethyl)benzoyl)-9-((S*)-1-(2-(difluoromethoxy) pyridin-4-yl)ethyl)-3,7-dimethyl-1,2,3,4,8,9-hexahydropyrido[4′,3′:3,4]pyrazolo[1,5-a]pyrazin-10(7H)-one (50B)

A racemic mixture of (3R,7R)-2-(4-chloro-3-(trifluoromethyl)benzoyl)-9-(1-(2-(difluoromethoxy)pyridin-4-yl)ethyl)-3,7-dimethyl-1,2,3,4,8,9-hexahydropyrido[4′,3′:3,4]pyrazolo[1,5-a]pyrazin-10(7H)-one 50 (150 mg, 100% purity, 0.251 mmol) was separated by Chiral Prep-HPLC (Column: Chiralpak IE 5 μm 30 mm*250 mm; Mobile Phase: Hex:EtOH=45:55 at 25 mL/min; Col. Temp: 30° C.; Wavelength: 230 nm) to afford the title compound 50A (44 mg, 99.4% purity from LCMS, 29% yield, 100% stereopure) as a white solid and 50B (58 mg, 99.9% purity from LCMS, 39% yield, 99.9% stereopure) as a white solid.

### 50A:

LC-MS (ESI): R_{T}=4.112 min, mass calcd. for C₂₇H₂₅ClF₅N₅O₃ 597.2, m/z found 598.2 [M+H]⁺. Chiral analysis (Column: Chiralpak IE 5 μm 4.6 mm*250 mm; Mobile Phase: Hex:EtOH=45:55 at 25 mL/min; Col. Temp: 30° C.; Wavelength: 230 nm; Rt=6.113 min). ¹H NMR (400 MHz, DMSO-d₆) δ 8.28-8.19 (m, 1H), 7.91-7.54 (m, 4H), 7.32-7.18 (m, 1H), 7.10-6.98 (m, 1H), 5.81-5.62 (m, 1H), 5.53-5.17 (m, 1H), 4.60-4.38 (m, 2H), 4.25-4.08 (m, 1H), 3.86-3.71 (m, 1H), 3.26-3.14 (m, 1H), 2.97-2.92 (m, 1H), 2.65-2.55 (m, 1H), 1.66-1.47 (m, 3H), 1.32 (d, J=6.4 Hz, 3H), 1.23-1.09 (m, 3H). ¹⁹F NMR (376 MHz, DMSO-d₆) δ −61.33, −87.32.

### 50B:

LC-MS (ESI): R_{T}=4.107 min, mass calcd. for C₂₇H₂₅ClF₅N₅O₃ 597.2, m/z found 598.2 [M+H]⁺. Chiral analysis (Column: Chiralpak IE 5 μm 4.6 mm*250 mm; Mobile Phase: Hex:EtOH=45:55 at 25 mL/min; Col. Temp: 30° C.; Wavelength: 230 nm; Rt=7.151 min). ¹H NMR (400 MHz, DMSO-d₆) δ 8.26-8.16 (m, 1H), 7.91-7.53 (m, 4H), 7.30-7.18 (m, 1H), 7.11-6.98 (m, 1H), 5.85-5.64 (m, 1H), 5.54-5.19 (m, 1H), 4.60-4.38 (m, 2H), 4.23-4.08 (m, 1H), 3.58-3.42 (m, 2H), 2.98-2.92 (m, 1H), 2.67-2.55 (m, 1H), 1.64-1.50 (m, 3H), 1.45 (d, J=6.4 Hz, 3H), 1.23-1.09 (m, 3H). ¹⁹F NMR (376 MHz, DMSO-d₆) δ −61.35, −87.31.

### Compounds 51A and 51B

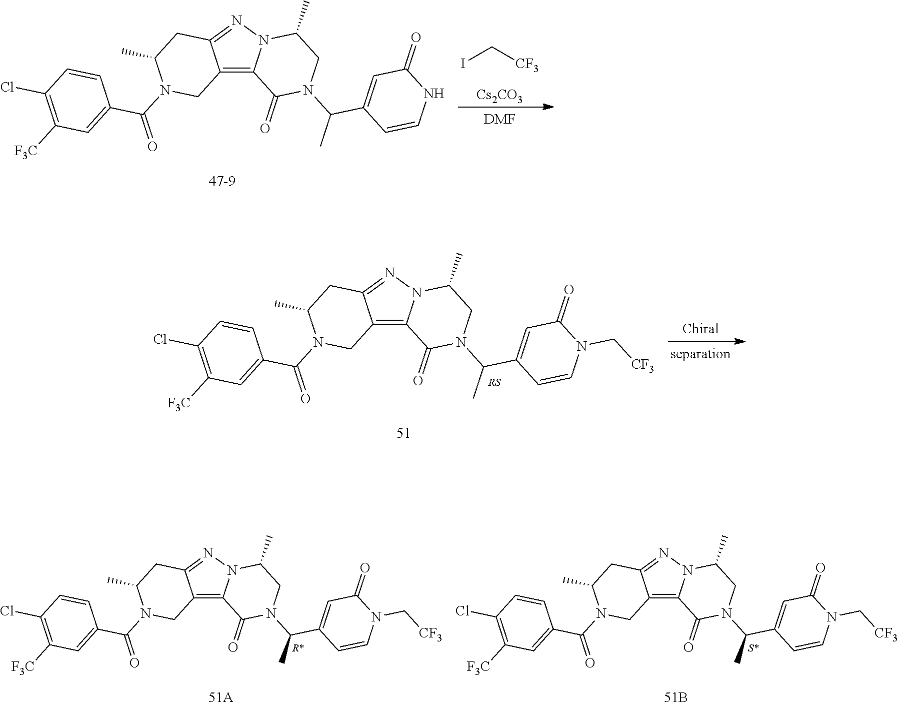

### Compound 51

### (3R,7R)-2-(3,4-dichlorobenzoyl)-3,7-dimethyl-9-(1-(2-oxo-1-(2,2,2-trifluoroethyl)-1,2-dihydropyridin-4-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-9-(1-(2-oxo-1,2-dihydropyridin-4-yl)ethyl)-1,2,3,4,8,9-hexahydropyrido[4′,3′:3,4]pyrazolo[1,5-a]pyrazin-10(7H)-one 47-9 (350 mg, 100% purity, 0.68 mmol) in N,N-dimethylformamide (4 mL) were added 1,1,1-trifluoro-2-iodoethane (158 mg, 0.75 mmol) and cesium carbonate (222 mg, 0.68 mmol) at room temperature. After addition, the vessel was then tightly sealed and heated at 80° C. for 18 h. The reaction mixture was quenched with water (20 mL) and extracted with ethyl acetate (20 mL) twice. The combined organic layers were dried over Na₂SO₄₍ₛ₎, filtered and concentrated. The residue was purified by silica gel flash chromatography (eluting gradient: 0-80% ethyl acetate in petroleum ether) to afford the title compound (230 mg, purity 98%, yield 56%) as white solids. LC-MS (ESI): R_{T}=1.478 min, mass calcd. for C₂₇H₂₆Cl₂F₃N₅O₃ 595.1 m/z found 596.0 [M+H]⁺.

### Compounds 51A and 51B

### (3R,7R)-2-(3,4-dichlorobenzoyl)-3,7-dimethyl-9-((R*)-1-(2-oxo-1-(2,2,2-trifluoroethyl)-1,2-dihydropyridin-4-yl)ethyl)-1,2,3,4,8,9-hexahydropyrido[4′,3′:3,4]pyrazolo[1,5-a]pyrazin-10(7H)-one (51A) and (3R,7R)-2-(3,4-dichlorobenzoyl)-3,7-dimethyl-9-((S*)-1-(2-oxo-1-(2,2,2-trifluoroethyl)-1,2-dihydropyridin-4-yl)ethyl)-1,2,3,4,8,9-hexahydropyrido[4′,3′:3,4]pyrazolo[1,5-a]pyrazin-10(7H)-one (51B)

A racemic mixture of (3R,7R)-2-(3,4-dichlorobenzoyl)-3,7-dimethyl-9-(1-(2-oxo-1-(2,2,2-trifluoroethyl)-1,2-dihydropyridin-4-yl)ethyl)-1,2,3,4,8,9-hexahydropyrido[4′,3′: 3,4]pyrazolo[1,5-a]pyrazin-10(7H)-one 51 (240 mg, 97% purity, 0.39 mmol) was separated by chiral HPLC (Column: Chiralpak IE, 5 m, 20*250 mm; Mobile Phase: Hex:EtOH=40:60 at 30 g/min; Temp: 30° C.; Wavelength: 254 nm) to afford the title compound 51A (65 mg, 28% yield, 99.9% purity from LCMS, 100% stereopure) as white solids and 51B (70 mg, 30% yield, 99.9% purity from LCM, 100% stereopure) as white solids.

### 51A:

LC-MS (ESI): R_{T}=3.776 min, mass calcd. for C₂₇H₂₆Cl₂F₃N₅O₃ 595.1 m/z found 596.1 [M+H]⁺. Chiral analysis (Column: Chiralpak IE 5 μm 4.6*250 mm; Mobile Phase: Hex:EtOH=40:60 at 1 mL/min; Temp: 30° C.; Wavelength: 254 nm; R_{T}=11.264 min). ¹H NMR (400 MHz, DMSO-d₆) δ 7.76-7.74 (m, 2H), 7.67-7.59 (m, 1H), 7.46-7.43 (m, 1H), 6.51-6.39 (m, 1H), 6.32-6.17 (m, 1H), 5.62-5.16 (m, 2H), 4.92-4.77 (m, 2H), 4.49 (br, s, 2H), 4.22-4.05 (m, 1H), 3.84-3.72 (m, 1H), 3.19 (br, s, 1H), 2.96-2.88 (m, 1H), 2.70-2.56 (m, 1H), 1.54-1.38 (m, 3H), 1.33 (d, J=6.0 Hz, 3H), 1.23-1.05 (m, 3H). ¹⁹F NMR (376 MHz, DMSO-d₆) δ −69.45.

### 51B:

LC-MS (ESI): R_{T}=3.793 min, mass calcd. for C₂₇H₂₆Cl₂F₃N₅O₃ 595.1 m/z found 595.9 [M+H]⁺. Chiral analysis (Column: Chiralpak IE 5 μm 4.6*250 mm; Mobile Phase: Hex:EtOH 5=40:60 at 1 mL/min; Temp: 30° C.; Wavelength: 254 nm; R_{T}=14.633 min). ¹H NMR (400 MHz, DMSO-d₆) δ 7.75-7.73 (m, 2H), 7.61 (br s, 1H), 7.46-7.43 (m, 1H), 6.50-6.37 (m, 1H), 6.32-6.17 (m, 1H), 5.66-5.18 (m, 2H), 4.92-4.74 (m, 2H), 4.62-4.07 (m, 3H), 3.50 (br s, 2H), 2.99-2.87 (m, 1H), 2.70-2.55 (m, 1H), 1.58-1.38 (m, 6H), 1.26-1.06 (m, 3H). ¹⁹F NMR (376 MHz, DMSO-d₆) δ −69.36.

### Compounds 52A and 52B

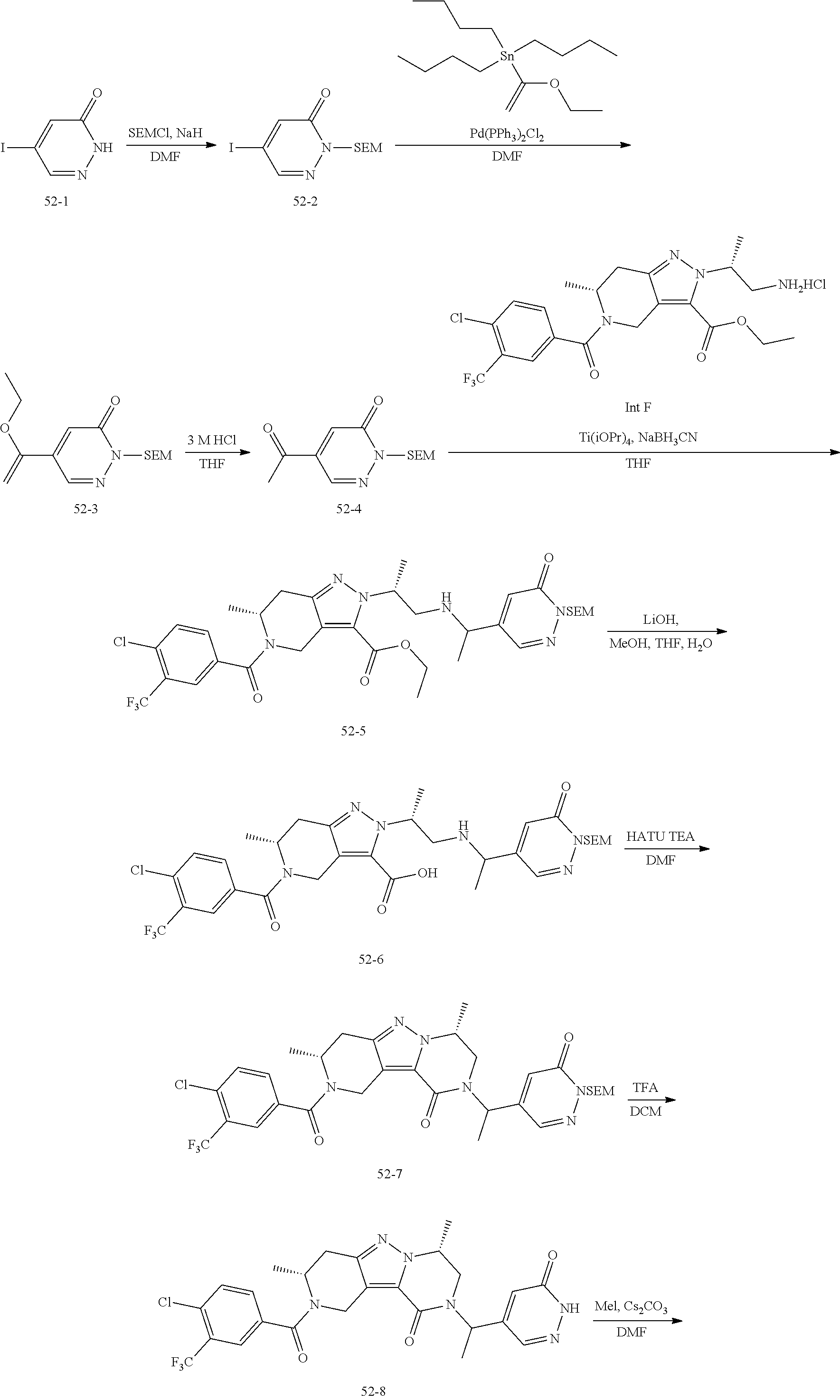

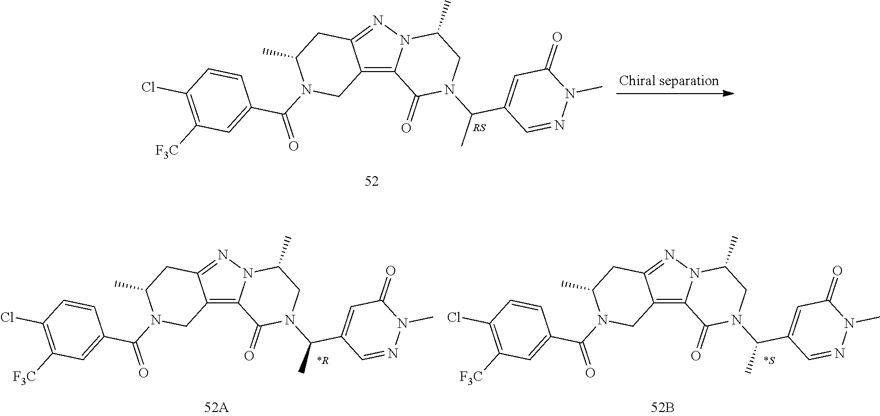

### Intermediate 52-2

### 5-Iodo-2-((2-(trimethylsilyl)ethoxy)methyl)pyridazin-3(2H)-one

To a solution of 5-iodopyridazin-3(2H)-one 52-1 (5 g, 22.5 mmol) in N,N-dimethylformamide (80 mL) was added sodium hydride (1.35 g, 60% purity, 33.8 mmol) slowly at 0° C. After completion, (2-(chloromethoxy)ethyl)trimethylsilane (6 mL, 33.9 mmol) was added to the solution. After stirred at 0° C. overnight, the mixture was added ethyl acetate (500 mL), washed with water (500 mL) and brine (500 mL) twice, dried over Na₂SO₄₍ₛ₎ and filtered. The filtrate was concentrated and purified by silica gel column chromatography (petroleum ether:ethyl acetate=5:1) to give title compound (5 g, 85% purity, 54% yield) as yellow oil. LC-MS (ESI): R_{T}=1.66 min, mass calcd. for C₁₀H₁₇IN₂02Si 352.0, m/z found 352.9 [M+H]⁺.

### Intermediate 52-3

### 5-(1-Ethoxyvinyl)-2-((2-(trimethylsilyl)ethoxy)methyl)pyridazin-3(2H)-one

To a solution of 5-iodo-2-((2-(trimethylsilyl)ethoxy)methyl)pyridazin-3(2H)-one 52-2 (5 g, 85% purity, 12.1 mmol) and tributyl(1-ethoxyvinyl)stannane (4.5 mL, 13.3 mmol) in N,N-dimethylformamide (50 mL) was added bis(triphenylphosphine)palladium(II) chloride (0.42 g, 0.60 mmol) under nitrogen atmosphere. After stirred at 80° C. overnight, the reaction mixture was quenched with saturated potassium fluoride (100 mL). The mixture was stirred at room temperature for 60 minutes, extracted with ether (150 mL) three times. The combined organic layers were washed with brine (300 mL), dried over Na₂SO₄₍ₛ₎, filtered. The filtrate was concentrated under reduced pressure to give crude title compound (5 g, 61% purity from LCMS, 85% yield) as yellow oil. LC-MS (ESI): R_{T}=1.81 min, mass calcd. for C₁₄H₂₄N₂O₃Si 296.2, m/z found 297.1 [M+H]⁺.

### Intermediate 52-4

### 5-Acetyl-2-((2-(trimethylsilyl)ethoxy)methyl)pyridazin-3(2H)-one

To a solution of 5-(1-ethoxyvinyl)-2-((2-(trimethylsilyl)ethoxy)methyl)pyridazin-3(2H)-one 52-3 (5 g, 61% purity, 10.3 mmol) in tetrahydrofuran (50 mL) was added 3 M Hydrochloric acid in water (50 mL, 150 mmol) at 0° C. After stirred at room temperature overnight, the reaction was quenched with saturated sodium carbonate to pH=9˜10 at 0° C. The aqueous layer was extracted with ethyl acetate (200 mL) twice. The combined organic layers were washed with brine (250 mL), dried over Na₂SO₄₍ₛ₎ and filtered. The filtrate was concentrated and purified by C18 chromatography (acetonitrile:water (+0.1% ammonium bicarbonate)=35%-55%) to give the title product (2.2 g, 80% purity from LCMS, 64% yield) as yellow solids. LC-MS (ESI): R_{T}=1.57 min, mass calcd. for C₁₂H₂₀N₂O₃Si 268.1, m/z found 269.0 [M+H]⁺.

### Intermediate 52-5

### Ethyl (6R)-5-(4-chloro-3-(trifluoromethyl)benzoyl)-6-methyl-2-((2R)-1-((1-(6-oxo-1-((2-(trimethylsilyl)ethoxy)methyl)-1,6-dihydropyridazin-4-yl)ethyl)amino)propan-2-yl)-4,5,6,7-tetrahydro-2H-pyrazolo[4,3-c]pyridine-3-carboxylate

To the solution of ethyl (R)-2-((R)-1-aminopropan-2-yl)-5-(4-chloro-3-(trifluoromethyl)benzoyl)-6-methyl-4,5,6,7-tetrahydro-2H-pyrazolo[4,3-c]pyridine-3-carboxylate Int F (1.9 μg, 95% purity, 3.54 mmol) and 5-acetyl-2-((2-(trimethylsilyl)ethoxy)methyl)pyridazin-3(2H)-one 52-4 (1.2 g, 80% purity, 3.58 mmol) in tetrahydrofuran (40 mL) was added tetraisopropoxytitanium (2.2 mL, 7.43 mmol) at room temperature. After stirred at room temperature for 1 hour, sodium cyanotrihydroborate (0.45 g, 7.28 mmol) was added to the mixture at 0° C. After stirred at 0° C. for 1 hour, the mixture was diluted with water (150 mL), extracted with ethyl acetate (150 mL) twice. The combined organic layers were washed with brine (150 mL), dried over Na₂SO₄₍ₛ₎, filtered. The filtrate was concentrated under reduced pressure to give crude (2.7 g, 78% purity from LCMS, 81% yield) as yellow oil. LC-MS (ESI): R_{T}=1.90 min, mass calcd. for C₃₃H₄₄ClF₃N₆O₅Si 724.3, m/z found 725.1 [M+H]⁺.

### Intermediate 52-6

### (6R)-5-(4-chloro-3-(trifluoromethyl)benzoyl)-6-methyl-2-((2R)-1-((1-(6-oxo-1-((2-(trimethylsilyl)ethoxy)methyl)-1,6-dihydropyridazin-4-yl)ethyl)amino) propan-2-yl)-4,5,6,7-tetrahydro-2H-pyrazolo[4,3-c]pyridine-3-carboxylic acid

To a solution of ethyl (6R)-5-(4-chloro-3-(trifluoromethyl)benzoyl)-6-methyl-2-((2R)-1-((1-(6-oxo-1-((2-(trimethylsilyl)ethoxy)methyl)-1,6-dihydropyridazin-4-yl)ethyl) amino)propan-2-yl)-4,5,6,7-tetrahydro-2H-pyrazolo[4,3-c]pyridine-3-carboxylate 52-5 (2.7 g, 78% purity, 2.90 mmol) in tetrahydrofuran (10 mL) and methanol (10 mL) was added lithium hydroxide monohydrate (0.41 g, 9.77 mmol) in water (10 mL) at 0° C. After stirred 0° C. for 5 hours, the mixture was added into 0.5 M hydrochloride aqueous solution (40 mL) and extracted with ethyl acetate (90 mL) twice. The combined organic layers were washed with water (90 mL), brine (90 mL), dried over Na₂SO₄₍ₛ₎ and filtered. The filtrate was concentrated under reduced pressure to give the title compound (2 g, 97% purity from LCMS, 96% yield) as yellow solids. LC-MS (ESI): R_{T}=1.175 min, mass calcd. for C₃₁H₄₀ClF₃N₆O₅Si 696.2, m/z found 697.1 [M+H]⁺.

### Intermediate 52-7

### (3R,7R)-2-(4-chloro-3-(trifluoromethyl)benzoyl)-3,7-dimethyl-9-(1-(6-oxo-1-((2-(trimethylsilyl)ethoxy)methyl)-1,6-dihydropyridazin-4-yl)ethyl)-1,2,3,4,8,9-hexahydropyrido[4′,3′:3,4]pyrazolo[1,5-a]pyrazin-10(7H)-one

To a mixture of (6R)-5-(4-chloro-3-(trifluoromethyl)benzoyl)-6-methyl-2-((2R)-1-((1-(6-oxo-1-((2-(trimethylsilyl)ethoxy)methyl)-1,6-dihydropyridazin-4-yl)ethyl)amino) propan-2-yl)-4,5,6,7-tetrahydro-2H-pyrazolo[4,3-c]pyridine-3-carboxylic acid 52-6 (2 g, 97% purity, 2.78 mmol) in N,N-dimethylformamide (30 mL) were added O-(7-azabenzotriazol-1-yl)-N,N,N′,N′-tetramethyluronium hexafluorophosphate (2.1 g, 5.52 mmol) and triethylamine (1.3 mL, 10.0 mmol) at 0° C. After stirred at 0° C. for 1 hour, the mixture was poured into water (100 mL), extracted with ethyl acetate (100 mL). The combined organic layers were washed with water (100 mL) twice, brine (100 mL), dried over Na₂SO₄₍ₛ₎ and filtered. The filtrate was concentrated and purified by C18 column (acetonitrile:water (+0.02% ammonium bicarbonate)=45% to 65%) to give the title compound (1.9 g, 88% purity from LCMS, 88% yield) as white solids. LC-MS (ESI): R_{T}=1.720 min, mass calcd. for C₃₁H₃₈ClF₃N₆O₄Si 678.2, m/z found 680.2 [M+H]⁺.

### Intermediate 52-8

### (3R,7R)-2-(4-chloro-3-(trifluoromethyl)benzoyl)-3,7-dimethyl-9-(1-(6-oxo-1,6-dihydropyridazin-4-yl)ethyl)-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-9-(1-(6-oxo-1-((2-(trimethylsilyl)ethoxy)methyl)-1,6-dihydropyridazin-4-yl)ethyl)-1,2,3,4,8,9-hexahydropyrido[4′,3′:3,4]pyrazolo[1,5-a]pyrazin-10(7H)-one 52-7 (1.9 g, 88% purity, 2.46 mmol) in dichloromethane (19 mL) was added 2,2,2-trifluoroacetic acid (19 mL, 248 mmol) at 0° C. After stirred at 0° C. for 2 hours, the reaction was quenched with saturated sodium carbonate aqueous solution to pH=9˜10 at 0° C. The aqueous layer was extracted with ethyl acetate (150 mL) twice. The combined organic layers were washed with brine (200 mL), dried over Na₂SO₄₍ₛ₎ and filtered. The filtrate was concentrated to give the title compound (1.3 g, 85% purity, 82% yield) as yellow solids. LC-MS (ESI): R_{T}=1.46 min, mass calcd. for C₂₅H₂₄ClF₃N₆O₃ 548.2, m/z found 549.0 [M+H]⁺.

### Compound 52

### (3R,7R)-2-(4-chloro-3-(trifluoromethyl)benzoyl)-3,7-dimethyl-9-(1-(1-methyl-6-oxo-1,6-dihydropyridazin-4-yl)ethyl)-1,2,3,4,8,9-hexahydropyrido[4′,3′:3,4]pyrazolo[1,5-a]pyrazin-10(7H)-one

To the solution of (3R,7R)-2-(4-chloro-3-(trifluoromethyl)benzoyl)-3,7-dimethyl-9-(1-(6-oxo-1,6-dihydropyridazin-4-yl)ethyl)-1,2,3,4,8,9-hexahydropyrido[4′,3′:3,4]pyrazolo[1,5-a]pyrazin-10(7H)-one 52-8 (200 mg, 85% purity, 0.31 mmol) in N,N-dimethylformamide (6 mL) were added cesium carbonate (205 mg, 0.63 mmol) and iodomethane (0.04 mL, 0.64 mmol) at 0° C. After stirred at room temperature overnight, the mixture was added water (30 mL), extracted with ethyl acetate (30 mL) twice. The combined organic layers were washed with brine (30 mL), dried over Na₂SO₄₍ₛ₎ and filtered. The filtrate was concentrated and purified by C18 chromatography (acetonitrile:water (+0.1% ammonium bicarbonate)=35%-55%) to give the title product (140 mg, 96% purity from LCMS, 77% yield) as white solids. LC-MS (ESI): R_{T}=1.152 min, mass calcd. for C₂₆H₂₆ClF₃N₆O₃ 562.2, m/z found 563.1 [M+H]⁺.

### Compounds 52A and 52B

### (3R,7R)-2-(4-chloro-3-(trifluoromethyl)benzoyl)-3,7-dimethyl-9-((R*)-1-(1-methyl-6-oxo-1,6-dihydropyridazin-4-yl)ethyl)-1,2,3,4,8,9-hexahydropyrido [4′,3′:3,4]pyrazolo[1,5-a]pyrazin-10(7H)-one (52A) and (3R,7R)-2-(4-chloro-3-(trifluoromethyl)benzoyl)-3,7-dimethyl-9-((S*)-1-(1-methyl-6-oxo-1,6-dihydropyridazin-4-yl)ethyl)-1,2,3,4,8,9-hexahydropyrido [4′,3′:3,4]pyrazolo[1,5-a]pyrazin-10(7H)-one (52B)

The racemic mixture of (3R,7R)-2-(4-chloro-3-(trifluoromethyl)benzoyl)-3,7-dimethyl-9-(1-(1-methyl-6-oxo-1,6-dihydropyridazin-4-yl)ethyl)-1,2,3,4,8,9-hexahydropyrido[4′,3′:3,4]pyrazolo[1,5-a]pyrazin-10(7H)-one 52 (210 mg, 96% purity, 0.36 mmol) was separated by chiral Prep. HPLC (separation condition: Column: Chiralpak IB N-5 m 20*250 mm; Mobile Phase: Hex:EtOH=40:60 at 15 mL/min; Temp: 30° C.; Wavelength: 254 nm) to afford peak1 (100 mg, 83.7% purity from SFC, 42% yield) and peak2 (80 mg, 82.4% purity from SFC, 33% yield) as white solids. Peak1 was separated by chiral Prep. HPLC (separation condition: Column: Chiralpak IB N-5 5 μm 20*250 mm; Mobile Phase: Hex:EtOH=40:60 at 15 mL/min; Temp: 30° C.; Wavelength: 254 nm) to afford the title compound 52A (75 mg, 99.1% purity, 89% yield, 100% stereopure) as white solids. Peak2 was separated by chiral Prep. HPLC (separation condition: Column: Chiralpak IB N-5 m 20*250 mm; Mobile Phase: Hex:EtOH=40:60 at 15 mL/min; Temp: 30° C.; Wavelength: 254 nm) to afford the title compound 52B (58 mg, 99.8% purity, 88% yield, 99.9% stereopure) as white solids.

### 52A:

LC-MS (ESI): R_{T}=3.908 min, mass calcd. for C₂₆H₂₆ClF₃N₆O₃ 562.2, m/z found 563.2 [M+H]⁺. Chiral analysis (Column: Chiralpak IB N-5 5 m 4.6*250 mm; Mobile Phase: Hex:EtOH=40:60 at 1 mL/min; Temp: 30° C.; Wavelength: 254 nm; Rt=9.530 min). ¹H NMR (400 MHz, CDCl₃) δ 7.78 (s, 1H), 7.72 (s, 1H), 7.59-7.52 (m, 2H), 6.85 (s, 1H), 5.91-5.38 (m, 2H), 4.79-4.25 (m, 3H), 3.77 (s, 3H), 3.65-3.61 (m, 1H), 3.08-3.03 (m, 2H), 2.69 (d, J=16.0 Hz, 1H), 1.51 (d, J=6.4 Hz, 3H), 1.43 (d, J=6.4 Hz, 3H), 1.29 (d, J=2.4 Hz, 3H).

### 52B:

LC-MS (ESI): R_{T}=3.958 min, mass calcd. for C₂₆H₂₆ClF₃N₆O₃ 562.2, m/z found 563.2 [M+H]⁺. Chiral analysis (Column: Chiralpak IB N-5 5 m 4.6*250 mm; Mobile Phase: Hex:EtOH=40:60 at 1 mL/min; Temp: 30° C.; Wavelength: 254 nm; Rt=12.253 min. ¹H NMR (400 MHz, CDCl₃) δ 7.80 (s, 1H), 7.65-7.53 (m, 3H), 6.83 (s, 1H), 5.92-5.38 (m, 2H), 4.84-4.29 (m, 3H), 3.77 (s, 3H), 3.37-3.28 (m, 2H), 3.11-2.68 (m, 2H), 1.59 (d, J=6.4 Hz, 3H), 1.52 (d, J=7.2 Hz, 3H), 1.27 (s, 3H).

### Compounds 53A and 53B

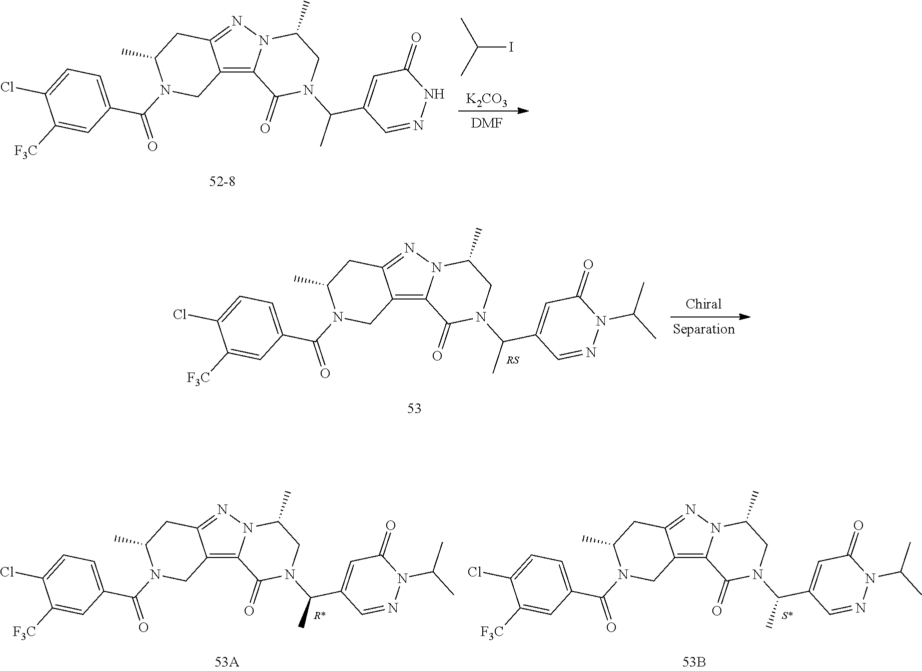

### Compound 53

### (3R,7R)-2-(4-chloro-3-(trifluoromethyl)benzoyl)-9-(1-(1-isopropyl-6-oxo-1,6-dihydropyridazin-4-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-9-(1-(6-oxo-1,6-dihydropyridazin-4-yl)ethyl)-1,2,3,4,8,9-hexahydropyrido[4′,3′:3,4]pyrazolo[1,5-a]pyrazin-10(7H)-one 52-8 (250 mg, 85% purity, 0.387 mmol) in N,N-dimethylformamide (3 mL) were added potassium carbonate (400 mg, 2.89 mmol) and 2-iodopropane (0.12 mL, 1.2 mmol) at 30° C. The reaction mixture was quenched with water (10 mL) and extracted with ethyl acetate (10 mL) twice. The combined organic layers were washed with brine (15 mL), dried over Na₂SO₄₍ₛ₎ and filtered. The filtrate was concentrated and purified by C18 column (acetonitrile:water=45% to 55%) to give the title compound (150 mg, 100% purity, 65.6% yield) as yellow oil. LC-MS (ESI): R_{T}=2.86 min, mass calcd. for C₂₈H₃₀ClF₃N₆O₃ 590.2, m/z found 591.0 [M+H]⁺.

### Compounds 53A and 53B

### (3R,7R)-2-(4-chloro-3-(trifluoromethyl)benzoyl)-9-((R*)-1-(1-isopropyl-6-oxo-1,6-dihydropyridazin-4-yl)ethyl)-3,7-dimethyl-1,2,3,4,8,9-hexahydropyrido[4′,3′:3,4]pyrazolo[1,5-a]pyrazin-10(7H)-one (53A) and (3R,7R)-2-(4-chloro-3-(trifluoromethyl)benzoyl)-9-((S*)-1-(1-isopropyl-6-oxo-1,6-dihydropyridazin-4-yl)ethyl)-3,7-dimethyl-1,2,3,4,8,9-hexahydropyrido[4′,3′:3,4]pyrazolo[1,5-a]pyrazin-10(7H)-one (53B)

The racemate of (3R,7R)-2-(4-chloro-3-(trifluoromethyl)benzoyl)-9-(1-(1-isopropyl-6-oxo-1,6-dihydropyridazin-4-yl)ethyl)-3,7-dimethyl-1,2,3,4,8,9-hexahydropyrido[4′,3′:3,4]pyrazolo[1,5-a]pyrazin-10(7H)-one 53 (150 mg, 100% purity, 0.254 mmol) was separated by chiral Prep. HPLC (Column: Chiralpak IC 5 m 30*250 mm; Mobile Phase: ACN:IPA=70:30 at 30 mL/min; Temp: 30° C.; Wavelength: 214 nm) to give the title compounds 53A (36.3 mg, 99.9% purity, 24.2% yield, 100% stereopure) as white solids and 53B (39.9 mg, 99.0% purity, 26.3% yield, 99.9% stereopure) as white solids.

### 53A:

LC-MS (ESI): R_{T}=3.695 min, mass calcd. for C₂₈H₃₀ClF₃N₆O₃ 590.2, m/z found 591.2 [M+H]⁺. Chiral analysis: (Column: Chiralpak IC 5 m 4.6*250 mm; Mobile Phase: ACN:IPA=70:30 at 1 mL/min; Temp: 30° C.; Wavelength: 214 nm; R_{T}=4.213 min). ¹H NMR (400 MHz, CDCl₃) δ 7.78 (s, 2H), 7.62-7.52 (m, 2H), 6.81 (s, 1H), 5.94-5.25 (m, 3H), 4.83-4.21 (m, 3H), 3.69-3.57 (m, 1H), 3.16-2.93 (m, 2H), 2.76-2.63 (m, 1H), 1.55-1.48 (m, 3H), 1.44-1.42 (m, 3H), 1.35-1.23 (m, 9H). ¹⁹F NMR (376 MHz, CDCl₃) δ −62.81.

### 54B:

LC-MS (ESI): R_{T}=3.691 min, mass calcd. for C₂₈H₃₀ClF₃N₆O₃ 590.2, m/z found 591.3 [M+H]⁺. Chiral analysis (Column: Chiralpak IC 5 m 4.6*250 mm; Mobile Phase: ACN:IPA=70:30 at 1 mL/min; Temp: 30° C.; Wavelength: 214 nm; R_{T}=5.295 min). ¹H NMR (400 MHz, CDCl₃) δ 7.80-7.69 (m, 2H), 7.60-7.53 (m, 2H), 6.83-6.76 (m, 1H), 5.97-5.23 (m, 3H), 4.93-4.37 (m, 3H), 3.36-3.34 (m, 2H), 3.17-2.96 (m, 1H), 2.72-2.66 (m, 1H), 1.64-1.60 (m, 3H), 1.53-1.51 (m, 3H), 1.35-1.24 (m, 9H). ¹⁹F NMR (376 MHz, CDCl₃) δ −62.80.

### Compounds 54A and 54B

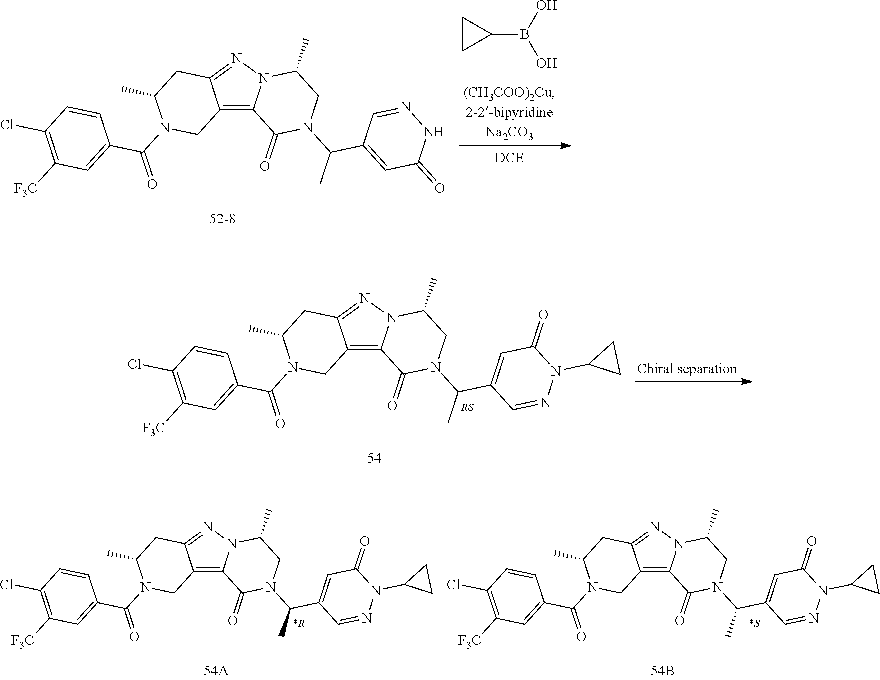

### Compound 54

### (3R,7R)-2-(4-chloro-3-(trifluoromethyl)benzoyl)-9-(1-(1-cyclopropyl-6-oxo-1,6-dihydropyridazin-4-yl)ethyl)-3,7-dimethyl-1,2,3,4,8,9-hexahydropyrido[4′,3′: 3,4]pyrazolo[1,5-a]pyrazin-10(7H)-one

To a solution of (3R,7R)-2-(4-chloro-3-(trifluoromethyl)benzoyl)-3,7-dimethyl-9-(1-(6-oxo-1,6-dihydropyridazin-4-yl)ethyl)-1,2,3,4,8,9-hexahydropyrido[4′,3′:3,4]pyrazolo[1,5-a]pyrazin-10(7H)-one 52-8 (200 mg, 85% purity, 0.31 mmol), sodium carbonate (68 mg, 0.64 mmol), cyclopropylboronic acid (85 mg, 0.99 mmol) and copper(II) acetate (60 mg, 0.33 mmol) in 1,2-dichloroethane (10 mL) was added 2,2′-bipyridine (51 mg, 0.33 mmol) at room temperature. Then the reaction mixture was stirred at 70° C. overnight. The reaction mixture was quenched with saturated ammonium chloride aqueous solution (10 mL), extracted with ethyl acetate (40 mL) twice. The combined organic layers were washed with brine (40 mL), dried over Na₂SO₄₍ₛ₎ and filtered. The filtrate was concentrated and purified by C18 chromatography (acetonitrile:water (+0.02% ammonium bicarbonate)=40%-65%) to give the title product (140 mg, 100% purity from LCMS, 77% yield) as a white solid. LC-MS (ESI): R_{T}=1.60 min, mass calcd. for C₂₈H₂₈ClF₃N₆O₃ 588.2, m/z found 589.0 [M+H]⁺.

### Compounds 54A and 54B

### (3R,7R)-2-(4-chloro-3-(trifluoromethyl)benzoyl)-9-((R*)-1-(1-cyclopropyl-6-oxo-1,6-dihydropyridazin-4-yl)ethyl)-3,7-dimethyl-1,2,3,4,8,9-hexahydropyrido[4′,3′: 3,4]pyrazolo[1,5-a]pyrazin-10(7H)-one (54A) and (3R,7R)-2-(4-chloro-3-(trifluoromethyl)benzoyl)-9-((S*)-1-(1-cyclopropyl-6-oxo-1,6-dihydropyridazin-4-yl)ethyl)-3,7-dimethyl-1,2,3,4,8,9-hexahydropyrido[4′,3′: 3,4]pyrazolo[1,5-a]pyrazin-10(7H)-one (54B)

A racemic mixture of (3R,7R)-2-(4-chloro-3-(trifluoromethyl)benzoyl)-9-(1-(1-cyclopropyl-6-oxo-1,6-dihydropyridazin-4-yl)ethyl)-3,7-dimethyl-1,2,3,4,8,9-hexahydropyrido[4′,3′: 3,4]pyrazolo[1,5-a]pyrazin-10(7H)-one 54 (200 mg, 100% purity, 0.34 mmol) was separated by chiral Prep. HPLC (separation condition: Column: Chiralpak IE 5 μm 20*250 mm; Mobile Phase: ACN:IPA=50:50 at 15 mL/min; Temp: 30° C.; Wavelength: 254 nm) to afford the title compounds 54A (65 mg, 99.9% purity, 31% yield, 100% stereopure) and 54B (48 mg, 99.9% purity, 23% yield, 99.9% stereopure) as white solids.

### 54A:

LC-MS (ESI): R_{T}=8.486 min, mass calcd. for C₂₈H₂₈ClF₃N₆O₃ 588.2, m/z found 589.2 [M+H]f. Chiral analysis (Column: Chiralpak IE 5 m 4.6*250 mm; Mobile Phase: ACN:IPA=50:50 at 1 mL/min; Temp: 30° C.; Wavelength: 254 nm; Rt=5.802 min). ¹H NMR (400 MHz, CDCl₃) δ 7.78 (s, 1H), 7.69 (s, 1H), 7.59-7.52 (m, 2H), 6.83 (s, 1H), 5.95-5.41 (m, 2H), 4.78-4.32 (m, 3H), 4.13-4.08 (m, 1H), 3.61 (d, J=14.0 Hz, 1H), 3.09-3.04 (m, 2H), 2.69 (d, J=15.2 Hz, 1H), 1.51 (d, J=6.8 Hz, 3H), 1.43 (d, J=6.4 Hz, 3H), 1.29 (d, J=5.2 Hz, 3H), 1.11-0.99 (m, 4H).

### 54B:

LC-MS (ESI): R_{T}=8.572 min, mass calcd. for C₂₈H₂₈ClF₃N₆O₃ 588.2, m/z found 589.2 [M+H]f. Chiral analysis (Column: Chiralpak IE 5 m 4.6*250 mm; Mobile Phase: ACN:IPA=50:50 at 1 mL/min; Temp: 30° C.; Wavelength: 254 nm; Rt=8.731 min. ¹H NMR (400 MHz, CDCl₃) δ 7.79 (s, 1H), 7.63-7.52 (m, 3H), 6.81 (s, 1H), 5.88-5.39 (m, 2H), 4.81-4.26 (m, 3H), 4.13-4.07 (m, 1H), 3.32 (d, J=7.6 Hz, 2H), 3.12-2.67 (m, 2H), 1.59 (d, J=6.4 Hz, 3H), 1.51 (d, J=6.8 Hz, 3H), 1.27 (d, J=4.8 Hz, 3H), 1.12-0.99 (m, 4H).

### Compounds 55A and 55B

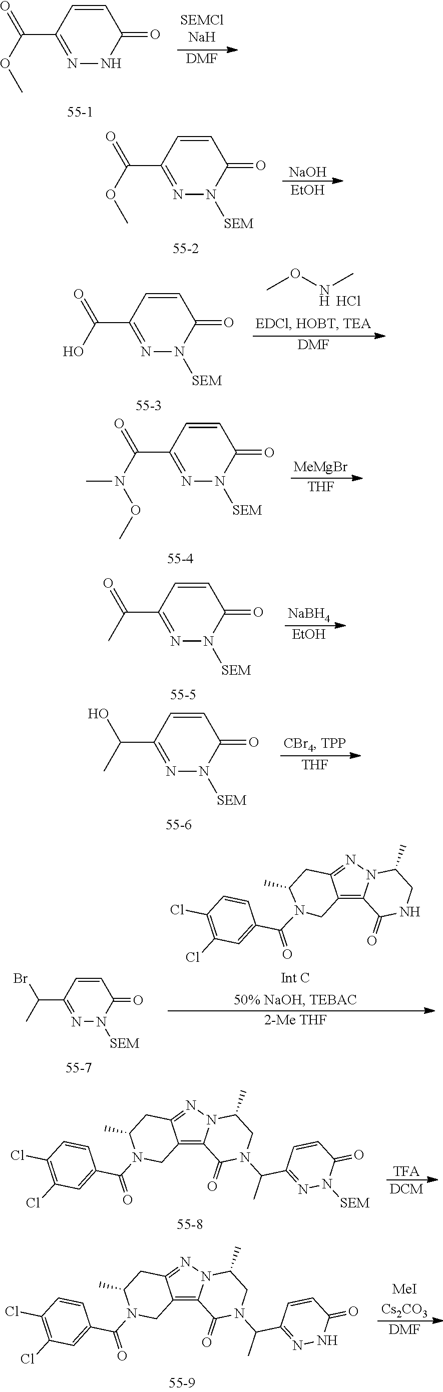

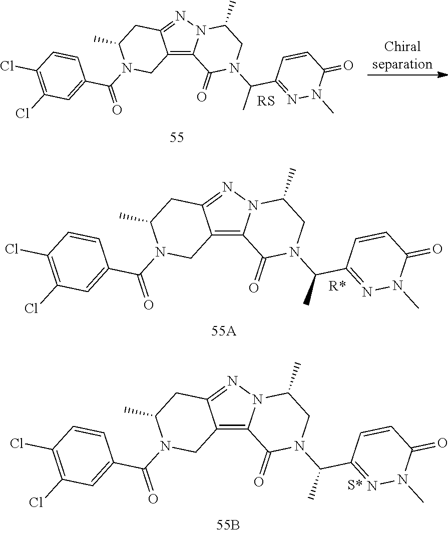 [SMILES: CC(c1ccc(=O)n(C)n1)N1C[C@@H](C)n2nc3c(c2C1=O)CN(C(=O)c1ccc(Cl)c(Cl)c1)[C@H](C)C3 ; "Chiralseparation" ; C[C@@H]1Cc2nn3c(c2CN1C(=O)c1ccc(Cl)c(Cl)c1)C(=O)N([C@H](C)c1ccc(=O)n(C)n1)C[C@H]3C ; C[C@@H]1Cc2nn3c(c2CN1C(=O)c1ccc(Cl)c(Cl)c1)C(=O)N([C@@H](C)c1ccc(=O)n(C)n1)C[C@H]3C]

### Intermediate 55-2

### Methyl 6-oxo-1-((2-(trimethylsilyl)ethoxy)methyl)-1,6-dihydropyridazine-3-carboxylate

To a solution of methyl 6-oxo-1,6-dihydropyridazine-3-carboxylate 55-1 (5.0 g, 32.44 mmol) in N,N-dimethylformamide (50 mL) was added sodium hydride (2.0 g, 60% purity, 50.01 mmol) at 0° C. The mixture was stirred at 0° C. for 0.5 hour. (2-(chloromethoxy)ethyl)trimethylsilane (8.6 ml, 48.59 mmol) was added and the reaction mixture was stirred at 0° C. for 2 hours. The reaction mixture was added to 100 mL of cold water, then was extracted with ethyl acetate (100 mL) three times. The organic layers were combined and dried over Na₂SO₄₍ₛ₎, filtered and concentrated under reduced pressure. The crude product was purified by silica gel column chromatography using 0-15% ethyl acetate/petroleum ether to afford the title compound (4.5 g, purity 90% from ¹H NMR, yield 44%) as yellow oil. ¹H NMR (400 MHz, DMSO-d₆) δ 7.86 (d, J=9.6 Hz, 1H), 7.07 (d, J=10.0 Hz, 1H), 5.40 (s, 2H), 3.87 (s, 3H), 3.67 (t, J=8.0 Hz, 2H), 0.87 (t, J=8.4 Hz, 2H), −0.03-−0.05 (m, 9H).

### Intermediate 55-3

### 6-oxo-1-((2-(trimethylsilyl)ethoxy)methyl)-1,6-dihydropyridazine-3-carboxylic acid

To a solution of methyl 6-oxo-1-((2-(trimethylsilyl)ethoxy)methyl)-1,6-dihydropyridazine-3-carboxylate 55-2 (2.0 g, 90% purity, 6.33 mmol) in ethanol (20 mL) was added 3 M sodium hydroxide (60 ml, 180 mmol) at 0° C. After stirred at room temperature for 1 hour, the mixture was diluted by water (40 mL), and then removed ethanol, acidized with 3 M hydrochloride aqueous solution to pH 4˜5, extracted with ethyl acetate (50 mL) for three times. The organic layers were combined, washed with brine (40 mL), dried over Na₂SO₄₍ₛ₎ and filtered. The filtrate was concentrated to give the title compound (1.5 g, 90% purity from ¹H NMR, 79% yield) as white solids. ¹H NMR (400 MHz, DMSO-d₆) δ 13.68 (br,s, 1H), 7.85 (d, J=9.6 Hz, 1H), 7.04 (d, J=10 Hz, 1H), 5.39 (s, 2H), 3.67 (t, J=8 Hz, 2H), 0.87 (t, J=8.4 Hz, 2H), −0.03-−0.05 (m, 9H).

### Intermediate 55-4

### N-Methoxy-N-methyl-6-oxo-1-((2-(trimethylsilyl)ethoxy)methyl)-1,6-dihydropyridazine-3-carboxamide

To a solution of 6-oxo-1-((2-(trimethylsilyl)ethoxy)methyl)-1,6-dihydropyridazine-3-carboxylic acid 55-3 (1.5 g, purity 90%, 4.99 mmol) in N,N-dimethylformamide (20 mL) were added N,O-dimethylhydroxylamine hydrochloride (731 mg, 7.49 mmol), 1H-benzo[d][1,2,3]triazol-1-ol (1.0 μg, 7.40 mmol), Ni-((ethylimino)methylene)-N₃,N₃-dimethylpropane-1,3-diamine hydrochloride (1.5 g, 7.83 mmol) and triethylamine (2 mL, 15.46 mmol) at 0° C. After stirred at room temperature overnight, the mixture was added ethyl acetate (30 mL) and washed with water (40 mL) twice, brine (30 mL), dried over Na₂SO₄₍ₛ₎ and filtered. The filtrate was concentrated under reduced pressure to give crude (1.3 g, 83% yield, 100% purity from LCMS) as yellow oil. LC-MS (ESI): R_{T}=1.57 min, mass calcd. for C₁₃H₂₃N₃O₄S₁ 313.2 m/z found 314.1[M+H]⁺.

### Intermediate 55-5

### 6-Acetyl-2-((2-(trimethylsilyl)ethoxy)methyl)pyridazin-3(2H)-one

To a solution of N-methoxy-N-methyl-6-oxo-1-((2-(trimethylsilyl)ethoxy)methyl)-1,6-dihydropyridazine-3-carboxamide 55-4 (1.3 g, purity 100%, 4.15 mmol) in tetrahydrofuran (20 ml) was added 3 M methylmagnesium bromide in tetrahydrofuran (2.8 ml, 8.4 mmol) at 0° C. After stirred at 0° C. for 2 hours, the mixture was diluted with water (30 mL). The mixture was extracted with ethyl acetate (40 mL) three times. The combined organic layers were washed with brine (20 mL), dried over Na₂SO₄₍ₛ₎, filtered and concentrated to give the crude product (470 mg, 90% purity from ¹H NMR, 38% yield) as blue oil. ¹H NMR (400 MHz, DMSO-d₆) δ 7.87 (d, J=9.6 Hz, 1H), 6.96 (d, J=9.6 Hz, 1H), 5.53 (s, 2H), 3.77 (t, J=8 Hz, 2H), 2.58 (s, 3H), 0.99 (t, J=8.4 Hz, 2H), 0.013-0.005 (m, 9H).

### Intermediate 55-6

### 6-(1-hydroxyethyl)-2-((2-(trimethylsilyl)ethoxy)methyl)pyridazin-3(2H)-one

To a solution of 6-acetyl-2-((2-(trimethylsilyl)ethoxy)methyl)pyridazin-3(2H)-one 55-5 (900 mg, purity 90%, 0.34 mmol) in methanol (10 ml) was added sodium borohydride (91 mg, 2.41 mmol) at 0° C. After stirred at 0° C. for 0.5 hour, the mixture was diluted with ammonium chloride (10 mL). The mixture was extracted with ethyl acetate (30 mL) four times. The combined organic layers were washed with brine (30 mL), dried over Na₂SO₄₍ₛ₎, filtered and concentrated to give the crude product (800 mg, 90% purity from ¹H NMR, 88% yield) as blue oil. ¹H NMR (400 MHz, DMSO-d₆) δ 7.35 (d, J=10 Hz, 1H), 6.96 (d, J=9.6 Hz, 1H), 5.44 (s, 2H), 4.80 (q, J=6.8 Hz, 1H), 3.72 (t, J=8.4 Hz, 2H), 2.81 (br, s, 1H), 1.48 (d, J=6.4 Hz, 3H), 0.96 (t, J=8.8 Hz, 2H), −0.01-−0.02 (m, 9H).

### Intermediate 55-7

### 6-(1-bromoethyl)-2-((2-(trimethylsilyl)ethoxy)methyl)pyridazin-3(2H)-one

To a solution of 6-(1-hydroxyethyl)-2-((2-(trimethylsilyl)ethoxy)methyl)pyridazin-3(2H)-one 55-6 (1.1 g, 90% purity, 3.66 mmol) in tetrahydrofuran (10 mL) was added carbon tetrabromide (2.5 g, 7.54 mmol) at 0° C. Then triphenylphosphine (2.0 g, 7.63 mmol) was added portionwise. The reaction mixture was stirred at room temperature for 3 hours. The solvent was removed. The residue was purified by silica gel column chromatography (petroleum ether:ethyl acetate=1:0 to 10:1) to afford product (1.2 g, 90% purity from ¹H NMR, 89% yield) as colorless oil. ¹H NMR (400 MHz, DMSO-d₆) δ 7.40 (d, J=9.6 Hz, 1H), 6.95 (d, J=9.6 Hz, 1H), 5.42 (s, 2H), 5.04 (q, J=6.8 Hz, 1H), 3.71 (t, J=8.4 Hz, 2H), 1.97 (d, J=7.2 Hz, 3H), 0.95 (t, J=8.4 Hz, 2H), −0.02 (s, 9H).

### Intermediate 55-8

### (3R,7R)-2-(3,4-dichlorobenzoyl)-3,7-dimethyl-9-(1-(6-oxo-1-((2-(trimethylsilyl)ethoxy)methyl)-1,6-dihydropyridazin-3-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (1.0 g, 95% purity, 2.42 mmol), 6-(1-bromoethyl)-2-((2-(trimethylsilyl)ethoxy)methyl)pyridazin-3(2H)-one 55-7 (1.1 g, 90% purity, 2.97 mmol) and N-benzyl-N,N-diethylethanaminium chloride (83 mg, 0.36 mmol) in 2-methyltetrahydrofuran (10 mL) was added 50% sodium hydroxide solution (5 mL) at 0° C. Then the reaction mixture was stirred at room temperature for 8 hours. The reaction mixture was quenched with water (30 mL) and extracted with ethyl acetate (30 mL) twice. The combined organic layers were dried over Na₂SO₄₍ₛ₎ and filtered. The filtrate was concentrated under reduced pressure to give crude, which was purified by silica gel column chromatography (dichloromethane:methanol=1:0 to 20:1) to afford product (1.4 g, 100% purity from LCMS, 90% of yield) as white solids. LC-MS (ESI): R_{T}=1.72 min, mass calcd. for C₃₀H₃₈Cl₂N₆O₄S₁ 644.2 m/z found 662.2[M+18]⁺.

### Intermediate 55-9

### (3R,7R)-2-(3,4-dichlorobenzoyl)-3,7-dimethyl-9-(1-(6-oxo-1,6-dihydropyridazin-3-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-9-(1-(6-oxo-1-((2-(trimethylsilyl)ethoxy)methyl)-1,6-dihydropyridazin-3-yl)ethyl)-1,2,3,4,8,9-hexahydropyrido[4′,3′:3,4]pyrazolo[1,5-a]pyrazin-10(7H)-one 55-8 (1.3 g, 100% purity, 2.01 mmol) in dichloromethane (20 mL) was added trifluoroacetic acid (10 mL) at 0° C. The mixture was stirred at room temperature for 1 hour. The reaction mixture was added saturated sodium bicarbonate aqueous solution (15 mL) and extracted with dichloromethane (30 mL) for three times. The combined organic layers were washed with brine (20 mL), then dried over Na₂SO₄₍ₛ₎, and filtered. The filtrate was concentrated under reduced pressure to give the title compound (970 mg, 98% purity from LCMS, 92% yield) as white solids. LC-MS (ESI): R_{T}=1.061 min, mass calcd. for C₂₄H₂₄Cl₂N₆O₃ 514.1 m/z found 515.1[M+H]⁺.

### Compounds 55A and 55B

### (3R,7R)-2-(3,4-dichlorobenzoyl)-3,7-dimethyl-9-(1-(1-methyl-6-oxo-1,6-dihydropyridazin-3-yl)ethyl)-1,2,3,4,8,9-hexahydropyrido[4′,3′:3,4]pyrazolo[1,5-a]pyrazin-10(7H)-one

The mixture of (3R,7R)-2-(3,4-dichlorobenzoyl)-3,7-dimethyl-9-(1-(6-oxo-1,6-dihydropyridazin-3-yl)ethyl)-1,2,3,4,8,9-hexahydropyrido[4′,3′:3,4]pyrazolo[1,5-a]pyrazin-10(7H)-one 55-9 (200 mg, 98% purity, 0.38 mmol), iodomethane (108 mg, 0.761 mmol) and cesium carbonate (186 mg, 0.57 mmol) in N,N-dimethylformamide (3 mL) was stirred at 25° C. for 17 hours. The reaction mixture was diluted with cold water (15 mL) and filtered. The filter cake was dried in vacuo to give the title compound (180 mg, purity 98.5%, 88% yield) as white solids. LC-MS (ESI): R_{T}=1.1.07 min, mass calcd. for C₂₅H₂₆Cl₂N₆O₃ 528.1 m/z found 529.1[M+H]⁺.

### Compounds 55A and 55B

### (3R,7R)-2-(3,4-dichlorobenzoyl)-3,7-dimethyl-9-((R*)-1-(1-methyl-6-oxo-1,6-dihydropyridazin-3-yl)ethyl)-1,2,3,4,8,9-hexahydropyrido[4′,3′:3,4]pyrazolo[1,5-a]pyrazin-10(7H)-one (55A) and (3R,7R)-2-(3,4-dichlorobenzoyl)-3,7-dimethyl-9-((S*)-1-(1-methyl-6-oxo-1,6-dihydropyridazin-3-yl)ethyl)-1,2,3,4,8,9-hexahydropyrido[4′,3′:3,4]pyrazolo[1,5-a]pyrazin-10(7H)-one (55B)

The racemic mixture of (3R,7R)-2-(3,4-dichlorobenzoyl)-3,7-dimethyl-9-(1-(1-methyl-6-oxo-1,6-dihydropyridazin-3-yl)ethyl)-1,2,3,4,8,9-hexahydropyrido[4′,3′:3,4]pyrazolo [1,5-a]pyrazin-10(7H)-one 55 (180 mg, 98.5% purity, 0.335 mmol) was separated by chiral HPLC (Column: Chiralpak IE, 5 m, 20*250 mm; Mobile Phase: ACN:IPA=60:40 at 30 g/min; Temp: 30° C.; Wavelength: 254 nm) to afford the title compounds 55A (75 mg, 41% yield, 96% purity from LCMS, stereopure 100%) and 55B (66 mg, 37% yield, 99.2% purity from LCMS, stereopure 100%) as white solids.

### 55A:

LC-MS (ESI): R_{T}=2.903 min, mass calcd. for C₂₅H₂₆Cl₂N₆O₃ 528.1 m/z found 529.2 [M+H]⁺. Chiral analysis (Column: Chiralpak IE 5 μm 4.6*250 mm; Mobile Phase: ACN:IPA=60:40 at 1.0 mL/min; Temp: 30° C.; Wavelength: 254 nm; R_{T}=9.630 min). ¹H NMR (400 MHz, DMSO-d₆) δ 7.75-7.73 (m, 2H), 7.45-7.35 (m, 2H), 6.92 (d, J=10.0 Hz, 1H), 5.75-5.20 (m, 2H), 4.49 (br, s, 2H), 4.24-4.07 (m, 1H), 3.77 (br, s, 1H), 3.65 (s, 3H), 3.27-3.15 (m, 1H), 2.97-2.88 (m, 1H), 2.49-2.41 (m, 1H), 1.52-1.39 (m, 3H), 1.31 (d, J=6.4 Hz, 3H), 1.21-1.06 (m, 3H).

### 55B:

LC-MS (ESI): R_{T}=3.021 min, mass calcd. for C₂₅H₂₆Cl₂N₆O₃ 528.1 m/z found 529.2 [M+H]⁺. Chiral analysis (Column: Chiralpak IE 5 μm 4.6*250 mm; Mobile Phase: ACN:IPA=60:40 at 1.0 mL/min; Temp: 30° C.; Wavelength: 254 nm; R_{T}=11.558 min). ¹H NMR (400 MHz, DMSO-d₆) δ 7.75-7.73 (m, 2H), 7.46-7.36 (m, 2H), 6.89 (br, s, 1H), 5.78-5.19 (m, 2H), 4.59-4.06 (m, 3H), 3.64 (s, 3H), 3.48 (br, s, 2H), 2.95-2.91 (m, 1H), 2.58-2.43 (m, 1H), 1.46-1.44 (m, 6H), 1.25-1.06 (m, 3H).

### Compounds 56A and 56B

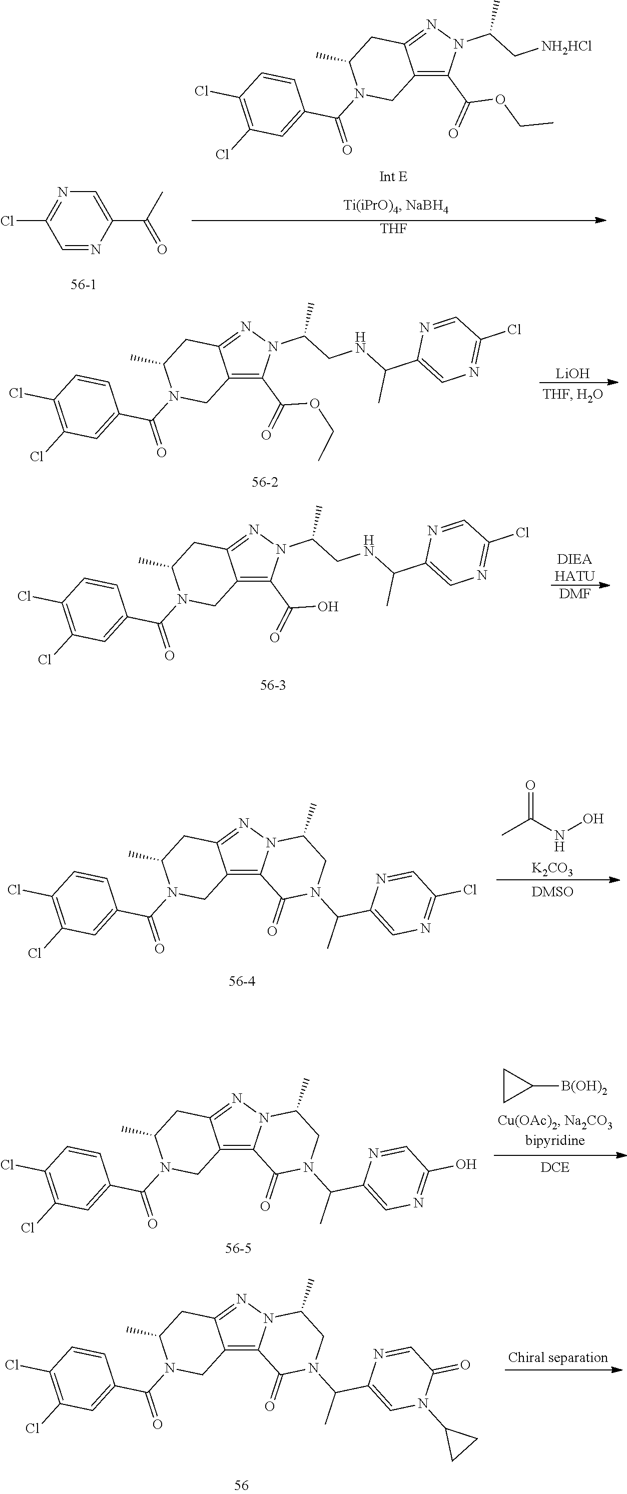

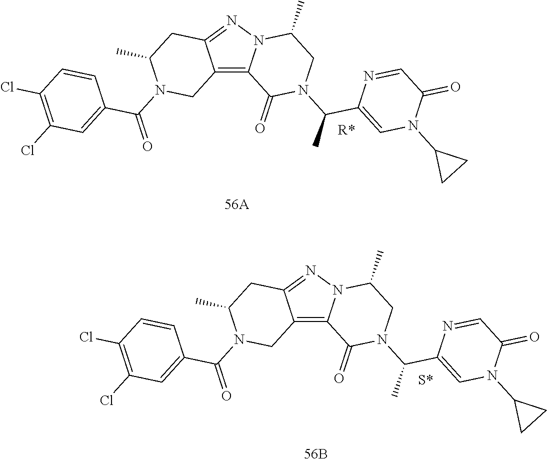 [SMILES: C[C@@H]1Cc2nn3c(c2CN1C(=O)c1ccc(Cl)c(Cl)c1)C(=O)N([C@H](C)c1cn(C2CC2)c(=O)cn1)C[C@H]3C ; C[C@@H]1Cc2nn3c(c2CN1C(=O)c1ccc(Cl)c(Cl)c1)C(=O)N([C@@H](C)c1cn(C2CC2)c(=O)cn1)C[C@H]3C]

### Intermediate 56-2

### Ethyl (6R)-2-((2R)-1-((1-(5-chloropyrazin-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylate

To a solution of 1-(5-chloropyrazin-2-yl)ethan-1-one 56-1 (324 mg, 2.07 mmol) and ethyl (R)-2-((R)-1-aminopropan-2-yl)-5-(3,4-dichlorobenzoyl)-6-methyl-4,5,6,7-tetrahydro-2H-pyrazolo[4,3-c]pyridine-3-carboxylate Int E (1.2 g, 100% purity, 2.52 mmol) in tetrahydrofuran (15 mL) was added titanium(IV) propan-2-olate (1.2 mL, 4.10 mmol). After stirred at 70° C. for 16 hours, the reaction mixture was cooled to 0° C. and sodium borohydride (157 mg, 4.15 mmol) was added. After stirred at 0° C. for 1 hour, the reaction was quenched with aqueous ammonium chloride solution (5 mL) and filtered with kieselguhr. The filtrate was concentrated to give the crude, which was purified by C18 column (acetonitrile:water=40% to 60%) to give the title compound (527 mg, 92% purity from LCMS, 40% yield) as yellow oil. LC-MS (ESI): R_{T}=1.86 min, mass calcd. for C₂₆H₂₉Cl₃N₆O₃ 578.1, m/z found 579.1 [M+H]⁺.

### Intermediate 56-3

### (6R)-2-((2R)-1-((1-(5-chloropyrazin-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid

To a solution of ethyl (6R)-2-((2R)-1-((1-(5-chloropyrazin-2-yl)ethyl)amino) propan-2-yl)-5-(3,4-dichlorobenzoyl)-6-methyl-4,5,6,7-tetrahydro-2H-pyrazolo[4,3-c]pyridine-3-carboxylate 56-2 (527 mg, 92% purity, 0.836 mmol) in tetrahydrofuran (6 mL) was added a solution of lithium hydroxide monohydrate (70 mg, 1.67 mmol) in water (2 mL) at 0° C. After stirred at 0° C. for 1 hour, the mixture was added into 0.5 M hydrochloride aqueous solution (2 mL). The mixture was concentrated under reduced pressure to give the title compound (500 mg, 71% purity, 77% yield) as yellow solids. LC-MS (ESI): R_{T}=1.36 min, mass calcd. for C₂₄H₂₅Cl₃N₆O₃ 550.1, m/z found 549.0 [M−H]⁻.

### Intermediate 56-4

### (3R,7R)-9-(1-(5-chloropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

The mixture of (6R)-2-((2R)-1-((1-(5-chloropyrazin-2-yl)ethyl)amino)propan-2-yl)-5-(3,4-dichlorobenzoyl)-6-methyl-4,5,6,7-tetrahydro-2H-pyrazolo[4,3-c]pyridine-3-carboxylic acid 56-3 (500 mg, 71% purity, 0.643 mmol), N-ethyl-N-isopropylpropan-2-amine (0.6 mL, 3.63 mmol), 2-(3H-[1,2,3]triazolo[4,5-b]pyridin-3-yl)-1,1,3,3-tetramethylisouronium hexafluorophosphate(V) (492 mg, 1.29 mmol) in N,N-dimethylformamide (10 mL) was stirred at room temperature for 14 hours. The mixture was acidified to pH=6 with 0.5 M hydrochloride aqueous solution and extracted with ethyl acetate (25 mL) twice. The combined organic layers were washed with water (25 mL) three times and brine (25 mL), dried over Na₂SO₄₍ₛ₎ and filtered. The filtrate was concentrated under reduced pressure to give the crude, which was purified by C18 column (acetonitrile:water=40% to 60%) to give the title compound (250 mg, 100% purity from LCMS, 73% yield) as yellow oil. LC-MS (ESI): R_{T}=1.65 min, mass calcd. for C₂₄H₂₃Cl₃N₆O₂ 532.1, m/z found 533.0 [M+H]⁺.

### Intermediate 56-5

### (3R,7R)-2-(3,4-dichlorobenzoyl)-9-(1-(5-hydroxypyrazin-2-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-9-(1-(5-chloropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 56-4 (180 mg, 100% purity, 0.337 mmol) in dimethyl sulfoxide (5 mL) was added acetylhydroxamic acid (76 mg, 1.01 mmol) and potassium carbonate (233 mg, 1.69 mmol). Then the reaction mixture was stirred at 80° C. under nitrogen atmosphere for 2 hours. The reaction mixture was diluted with water (20 mL), extracted with ethyl acetate (20 mL) twice. The combined organic layers were dried over Na₂SO₄₍ₛ₎ and filtered. The filtrate was concentrated under reduced pressure to give the title compound (145 mg, 89% purity from LCMS, 74% yield) as yellow solids. LC-MS (ESI): R_{T}=1.43 min, mass calcd. for C₂₄H₂₄Cl₂N₆O₃ 514.1, m/z found 515.0 [M+H]⁺.

### Compound 56

### (3R,7R)-9-(1-(4-cyclopropyl-5-oxo-4,5-dihydropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-9-(1-(5-hydroxypyrazin-2-yl)ethyl)-3,7-dimethyl-1,2,3,4,8,9-hexahydropyrido[4′,3′:3,4]pyrazolo[1,5-a]pyrazin-10(7H)-one 56-5 (150 mg, 89% purity, 0.259 mmol), copper(II) acetate (53 mg, 0.292 mmol), sodium carbonate (56 mg, 0.528 mmol) and cyclopropylboronic acid (68 mg, 0.792 mmol) in 1,2-dichloroethane (12 mL) was added 2,2′-bipyridine (45 mg, 0.288 mmol) at 0° C. Then the reaction mixture was stirred at 70° C. under nitrogen atmosphere for 16 hours. The reaction mixture was quenched with saturated ammonium chloride aqueous solution (15 mL) and extracted with dichloromethane (15 mL) twice. The combined organic layers were dried over Na₂SO₄₍ₛ₎ and filtered. The filtrate was concentrated under reduced pressure to give crude, which was purified by C18 column (acetonitrile:water=40% to 60%) to give the title compound (75 mg, 96% purity from LCMS, 50% yield) as yellow solids. LC-MS (ESI): R_{T}=1.50 min, mass calcd. for C₂₇H₂₈Cl₂N₆O₃ 554.2, m/z found 555.1 [M+H]⁺.

### Compounds 56A and 56B

### (3R,7R)-9-((R*)-1-(4-cyclopropyl-5-oxo-4,5-dihydropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (56A) and (3R,7R)-9-((S*)-1-(4-cyclopropyl-5-oxo-4,5-dihydropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one (56B)

A racemate of (3R,7R)-9-(1-(4-cyclopropyl-5-oxo-4,5-dihydropyrazin-2-yl)ethyl)-2-(3,4-dichlorobenzoyl)-3,7-dimethyl-1,2,3,4,8,9-hexahydropyrido[4′,3′:3,4]pyrazolo[1,5-a]pyrazin-10(7H)-one 56 (90 mg, 96% purity, 0.156 mmol) was separated by chiral Prep. HPLC separation condition: (Column: Chiralpak IE 5 μm 20*250 mm; Mobile Phase: ACN:IPA=70: at 30 mL/min; Temp: 30° C.; Wavelength: 214 nm) to give the title compounds 56A (26.3 mg, 99.8% purity, 30.4% yield, 100% stereopure) as a white solid and 56B (8.4 mg, 99.8% purity, 9.7% yield, 99.8% stereopure) as a white solid.

### 56A:

LC-MS (ESI): R_{T}=4.026 min, mass calcd. for C₂₇H₂₈Cl₂N₆O₃ 554.2, m/z found 555.2 [M+H]⁺. Chiral analysis (Column: Chiralpak IE 5 m 4.6*250 mm; Mobile Phase: ACN:IPA=70:30 at 30 mL/min; Temp: 30° C.; Wavelength: 214 nm; R_{T}=11.524 min). ¹H NMR (400 MHz, CDCl₃) δ 8.09 (s, 1H), 7.53-7.49 (m, 2H), 7.26-7.21 (m, 2H), 5.68-5.40 (m, 2H), 4.81-4.30 (m, 3H), 3.89-3.85 (m, 1H), 3.60 (br s, 1H), 3.42-3.29 (m, 1H), 3.04 (br s, 1H), 2.68-2.63 (m, 1H), 1.55-1.54 (m, 3H), 1.43-1.41 (m, 3H), 1.25-1.16 (m, 5H), 0.90 (s, 2H).

### 56B:

LC-MS (ESI): R_{T}=3.128 min, mass calcd. for C₂₇H₂₈Cl₂N₆O₃ 554.2, m/z found 555.2 [M+H]⁺. Chiral analysis (Column: Chiralpak IE 5 m 4.6*250 mm; Mobile Phase: ACN:IPA=70:30 at 30 mL/min; Temp: 30° C.; Wavelength: 214 nm; R_{T}=15.252 min). ¹H NMR (400 MHz, CDCl₃) δ 8.07 (s, 1H), 7.51-7.49 (m, 2H), 7.23-7.14 (m, 2H), 5.69-5.41 (m, 2H), 4.80-4.37 (m, 3H), 3.84-3.81 (m, 1H), 3.59 (br s, 1H), 3.30 (br s, 1H), 3.02 (br s, 1H), 2.68-2.64 (m, 1H), 1.58-1.44 (s, 6H), 1.26 (s, 3H), 1.18-1.16 (m, 2H), 0.88 (s, 2H).

### Compounds 57A and 57B

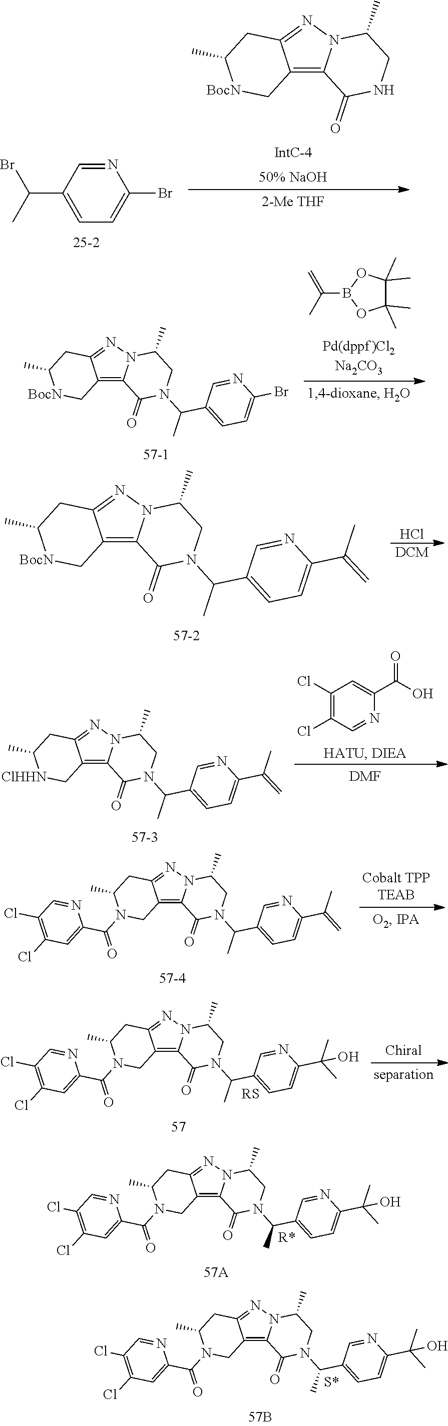

### Intermediate 57-1

### tert-Butyl (3R,7R)-9-(1-(6-bromopyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate

To a solution of tert-butyl (3R,7R)-3,7-dimethyl-10-oxo-3,4,7,8,9,10-hexahydropyrido[4′,3′:3,4]pyrazolo[1,5-a]pyrazine-2(1H)-carboxylate IntC-4 (4.5 g, 95% purity, 13.3 mmol) and 2-bromo-5-(1-bromoethyl)pyridine 25-2 (4.0 g, 15.1 mmol) in 2-methyl tetrahydrofuran (40 mL) was added 50% wt. sodium hydroxide in water (40 mL) slowly at 30° C. After stirred at 30° C. for 2 hours, the mixture was diluted with water (120 mL) and concentrated at room temperature under reduced pressure to remove the volatile. The remained aqueous layer was acidified with 2 M hydrochloride aqueous solution (200 mL) and extracted with ethyl acetate (100 mL) twice and brine (200 mL), dried over Na₂SO₄₍ₛ₎ and filtered. The filtrate was concentrated to give the crude and purified by C18 column (acetonitrile:water=5% to 100%) to give the title compound (7.0 g, 100% purity, 100% yield) as a yellow solid.

LC-MS (ESI): R_{T}=1.61 min, mass calcd. for C₂₃H₃₀BrN₅O₃ 503.2, m/z found 449.9 [M-56+H]⁺.

### Intermediate 57-2

### tert-Butyl (3R,7R)-3,7-dimethyl-10-oxo-9-(1-(6-(prop-1-en-2-yl)pyridin-3-yl) ethyl)-3,4,7,8,9,10-hexahydropyrido[4′,3′:3,4]pyrazolo[1,5-a]pyrazine-2(1H)-carboxylate

Tert-butyl (3R,7R)-9-(1-(6-bromopyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate 57-1 (1.0 g, 100% purity, 1.98 mmol), 4,4,5,5-tetramethyl-2-(prop-1-en-2-yl)-1,3,2-dioxaborolane (700 mg, 4.17 mmol), sodium carbonate (500 mg, 4.72 mmol) and 1,1′-bis(diphenylphosphino)ferrocene]dichloropalladium(II) (100 mg, 0.137 mmol) were mixed in 1,4-dioxane (12 mL) and water (2 mL). After stirred at 85° C. for 16 hours, the reaction mixture was poured into water and extracted with ethyl acetate. The ethyl acetate layer was washed with water, dried over Na₂SO₄₍ₛ₎ and filtered. The filtrate was concentrated to give the crude and purified by silica gel column chromatography (petroleum ether:ethyl acetate=1:1) to give the title compound (300 mg, 97% purity, 32% yield) as yellow oil. LC-MS (ESI): R_{T}=1.64 min, mass calcd. for C₂₆H₃₅N₅O₃ 465.3, m/z found 466.1 [M+H]⁺.

### Intermediate 57-3

### (3R,7R)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one hydrochloride

A solution of tert-butyl (3R,7R)-3,7-dimethyl-10-oxo-9-(1-(6-(prop-1-en-2-yl)pyridin-3-yl)ethyl)-3,4,7,8,9,10-hexahydropyrido[4′,3′:3,4]pyrazolo[1,5-a]pyrazine-2(1H)-carboxylate 57-2 (300 mg, 0.625 mmol, 97% purity) in dichloromethane (5 ml) was added 4 M hydrochloride in ethyl acetate (3 mL, 12 mmol). After stirred at 0° C. for 2 hours, the mixture was concentrated to give the title compound (250 mg, 99% yield, 100% purity) as yellow solids.

LC-MS (ESI): R_{T}=1.32 min, mass calcd. for C₂₁H₂₇N₅O 365.2, m/z found 366.1 [M+H]⁺.

### Intermediate 57-4

### (3R,7R)-2-(4,5-dichloropicolinoyl)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one

(3R,7R)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one hydrochloride 57-3 (80 mg, 0.199 mmol, 100% purity), 4,5-dichloropicolinic acid (45 mg, 0.234 mmol), N-ethyl-N-isopropylpropan-2-amine (0.25 mL, 1.35 mmol) and O-(7-azabenzotriazol-1-yl)-N,N,N′,N′-tetramethyluronium hexafluorophosphate (100 mg, 0.263 mmol) were mixed in N,N-dimethylformamide (1.5 mL) at 0° C. After stirred at 0° C. for 1 hour, the mixture was concentrated and purified by C18 column (acetonitrile:water (+0.02% ammonium acetate)=5% to 100%) to give the title compound (90 mg, 85% purity, 71% yield) as while solids. LC-MS (ESI): R_{T}=1.61 min, mass calcd. for C₂₇H₂₈Cl₂N₆O₂ 538.2, m/z found 539.1 [M+H]⁺.

### Compound 57

### (3R,7R)-2-(4,5-dichloropicolinoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To a mixture of (3R,7R)-2-(4,5-dichloropicolinoyl)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one 57-4 (150 mg, 100% purity, 0.278 mmol) in isopropyl alcohol (3 mL) was added 5,10,15,20-tetraphenyl-21H,23H-porphine cobalt (II) (10 mg, 0.015 mmol) and tetraethylammonium borohydride (70 mg, 0.482 mmol) at room temperature. After stirred at room temperature for 1.5 hours under dioxygen atmosphere, the mixture was purified by C18 column (acetonitrile:water=5% to 60%) to the title compound (110 mg, 84% purity from LCMS, 60% yield) as white solids. LC-MS (ESI): R_{T}=1.26 min, mass calcd. for C₂₇H₃₀Cl₂N₆O₃ 556.2, m/z found 557.0 [M+H]⁺ Compounds 57A and 57B

### (3R,7R)-2-(4,5-dichloropicolinoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (57A) and (3R,7R)-2-(4,5-dichloropicolinoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (57B)

The racemic (3R,7R)-2-(4,5-dichloropicolinoyl)-9-(1-(6-(2-hydroxypropan-2-yl) pyridin-3-yl)ethyl)-3,7-dimethyl-1,2,3,4,8,9-hexahydropyrido[4′,3′:3,4]pyrazolo[1,5-a]pyrazin-10(7H)-one 57 (180 mg, 84% purity, 0.271 mmol) was separated by chiral Prep. HPLC (separation method: Column: Chiralpak IF, 5 m 30*250 mm; Mobile Phase: acetonitrile:isopropyl alcohol:diethylamine=50:50:0.2 at 60 mL/min; Col. Temp: 30° C.; Wavelength: 254 nm) to give the title product 57A (52.6 mg, 97.9% purity, 34% yield, 100% stereopure) as a white solid and 57B (46.5 mg, 99.3% purity, 31% yield, 99.8% stereopure) as a white solid.

### 57A:

LC-MS (ESI): R_{T}=3.212 min, mass calcd. for C₂₇H₃₀Cl₂N₆O₃ 556.2, m/z found 557.1 [M+H]⁺. Chiral analysis (Column: Chiralpak IF, 5 m 4.6*250 mm; Mobile Phase: acetonitrile:isopropyl alcohol:diethylamine=50:50:0.2 at 1.0 mL/min; Col. Temp: 30° C.; Wavelength: 254 nm; R_{T}=5.085 min). ¹H NMR (400 MHz, CDCl₃) δ 8.61 (d, J=9.6 Hz, 1H), 8.55 (d, J=15.2 Hz, 1H), 7.80-7.68 (m, 2H), 7.41-7.38 (m, 1H), 6.20-6.05 (m, 1H), 5.77-5.43 (m, 1H), 5.05 (d, J=17.2 Hz, 1H), 4.79-4.62 (m, 2H), 4.46-4.33 (m, 1H), 3.71-3.58 (m, 1H), 3.19-2.98 (m, 2H), 2.74-2.59 (m, 1H), 1.67-1.60 (m, 3H), 1.56-1.51 (m, 6H), 1.33-1.24 (m, 6H).

### 57B:

LC-MS (ESI): R_{T}=2.913 min, mass calcd. for C₂₇H₃₀Cl₂N₆O₃ 556.2, m/z found 557.0 [M+H]⁺. Chiral analysis (Column: Chiralpak IF, 5 m 4.6*250 mm; Mobile Phase: acetonitrile:isopropyl alcohol:diethylamine=50:50:0.2 at 1.0 mL/min; Col. Temp: 30° C.; Wavelength: 254 nm; R_{T}=6.333 min). ¹H NMR (400 MHz, CDCl₃) δ 8.61 (d, J=10.4 Hz, 1H), 8.51 (d, J=17.6 Hz, 1H), 7.78 (d, J=8.4 Hz, 1H), 7.72-7.62 (m, 1H), 7.39-7.35 (m, 1H), 6.20-6.02 (m, 1H), 5.80-5.44 (m, 1H), 5.07 (d, J=17.6 Hz, 1H), 4.81-4.62 (m, 2H), 4.39-4.23 (m, 1H), 3.36-3.24 (m, 2H), 3.20-3.09 (m, 1H), 2.75-2.60 (m, 1H), 1.68-1.62 (m, 3H), 1.55-1.50 (m, 9H), 1.32-1.24 (m, 3H).

### Compounds 58A and 58B

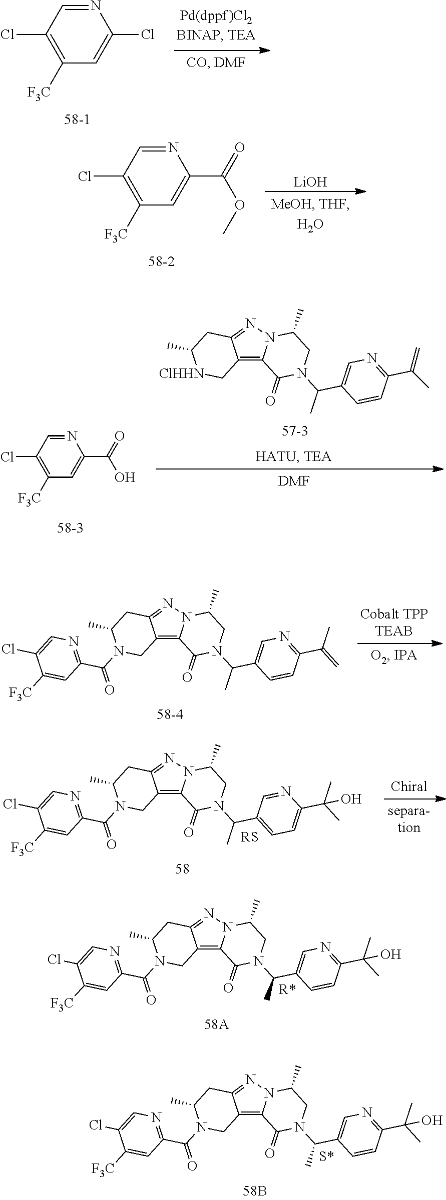

### Intermediate 58-2

### Methyl 5-chloro-4-(trifluoromethyl)picolinate

To a solution of 2,5-dichloro-4-(trifluoromethyl)pyridine 58-1 (2 g, 9.26 mmol), 2,2′-Bis-(diphenylphosphino)-1,1′-binaphthyl (570 mg, 0.915 mmol) in N,N-dimethylformamide (20 mL) was added triethylamine (6 mL, 43.2 mmol) and 1,1′-bis(diphenylphosphino)ferrocene]dichloropalladium(II) (800 mg, 1.09 mmol) at 25° C. After stirred at 60° C. under carbon monoxide atmosphere (50 psi) for 12 hours, the mixture was concentrated and purified by C18 column (acetonitrile:water=55% to 60%) to give the title compound (1.0 g, 92% purity from LCMS, 41.5% yield) as yellow solids. LC-MS (ESI): R_{T}=1.48 min, mass calcd. for C₈H₅ClF₃NO₂ 239.0, m/z found 239.9 [M+H]⁺.

### Intermediate 58-3

### 5-chloro-4-(trifluoromethyl)picolinic acid

To a solution of methyl 5-chloro-4-(trifluoromethyl)picolinate 58-2 (1.0 g, 92%, purity, 3.84 mmol) in tetrahydrofuran (5 mL) and methanol (5 mL) was added a solution of lithium hydroxide monohydrate (550 mg, 13.1 mmol) in water (1 mL) at 0° C. After stirred 25° C. for 4 hour, the mixture was diluted with water (20 mL) and adjusted the pH to 5˜6 by using 1 M hydrochloride aqueous solution, extracted with ethyl acetate (20 mL) twice. The combined organic layers were washed with brine (20 mL), dried over Na₂SO₄₍ₛ₎ and filtered. The filtrate was concentrated to give the title compound (800 mg, 68% purity, 62.8% yield) as yellow solids. LC-MS (ESI): R_{T}=0.28 min, mass calcd. for C₇H₃ClF₃NO₂ 225.0, m/z found 225.9 [M+H]⁺.

### Intermediate 58-4

### (3R,7R)-2-(5-chloro-4-(trifluoromethyl)picolinoyl)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one

To the solution of (3R,7R)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one hydrochloride 57-3 (460 mg, 86% purity, 0.984 mmol), 4-chloro-3,5-difluorobenzoic acid 58-3 (450 mg, 68% purity, 1.36 mmol) and 2-(3H-[1,2,3]triazolo[4,5-b]pyridin-3-yl)-1,1,3,3-tetramethylisouronium hexafluorophosphate(V) (500 mg, 1.32 mmol) in N,N-dimethylformamide (8 mL) was added triethylamine (0.6 mL, 4.64 mmol) at room temperature. After stirred at room temperature for 1 hour, the mixture was quenched with 0.5 M hydrochloride aqueous solution (15 mL) and extracted with ethyl acetate (20 mL) twice. The combined organic layers were washed with brine (20 mL), dried over Na₂SO₄₍ₛ₎ and filtered. The filtrate was concentrated and purified by C18 column (acetonitrile:water=50% to 60%) to give the title compound (500 mg, 71.6% purity, 63.5% yield) as white solids. LC-MS (ESI): R_{T}=1.45 min, mass calcd. for C₂₈H₂₈ClF₃N₆O₂ 572.2, m/z found 573.1 [M+H]⁺.

### Compound 58

### (3R,7R)-2-(5-chloro-4-(trifluoromethyl)picolinoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(5-chloro-4-(trifluoromethyl)picolinoyl)-3,7-dimethyl-9-(1-(6-(prop-1-en-2-yl)pyridin-3-yl)ethyl)-1,2,3,4,8,9-hexahydropyrido[4′,3′:3,4]pyrazolo[1,5-a]pyrazin-10(7H)-one 58-4 (500 mg, 71.6% purity, 0.625 mmol) in propan-2-ol (6 mL) was added 5,10,15,20-tetraphenyl-21H,23H-porphine cobalt (II) (30 mg, 0.045 mmol) and tetraethylammonium borohydride (130 mg, 0.998 mmol) at 15° C. under dioxygen atmosphere. After stirred for 3 hours, the reaction mixture was diluted with water (10 mL) and extracted with ethyl acetate (10 mL) three times. The organic layers were washed with brine (10 mL), dried over Na₂SO₄₍ₛ₎ and filtered. The filtrate was concentrated and purified by C18 column (acetonitrile:water=5% to 95%) to give the title compound (300 mg, 93% purity from LCMS, 75.6% yield) as yellow solids. LC-MS (ESI): R_{T}=2.77 min and 2.89 min, mass calcd. for C₂₈H₃₀ClF₃N₆O₃ 590.2, m/z found 591.0 [M+H]⁺.

### Compounds 58A and 58B

### (3R,7R)-2-(5-chloro-4-(trifluoromethyl)picolinoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (58A) and (3R,7R)-2-(5-chloro-4-(trifluoromethyl)picolinoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (58B)

The racemate of (3R,7R)-2-(5-chloro-4-(trifluoromethyl)picolinoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one 58 (300 mg, 93% purity, 0.472 mmol) was separated by chiral Prep. HPLC (Column: Chiralpak IC 5 m 30*250 mm; Mobile Phase: ACN:DEA=100:0.2 at 30 mL/min; Temp: 30° C.; Wavelength: 254 nm) to give the title compounds 58A (40.6 mg, 99.5% purity, 14.5% yield, 100% stereopure) as white solids and 58B (32.2 mg, 99.7% purity, 22.6% yield, 99.6% stereopure) as white solids.

### 58A:

LC-MS (ESI): R_{T}=3.085 min, mass calcd. for C₂₈H₃₀ClF₃N₆O₃ 590.2, m/z found 591.1 [M+H]⁺. Chiral analysis (Column: Chiralpak IC 5 m 4.6*250 mm; Mobile Phase: ACN:DEA=100:0.2 at 1 mL/min; Temp: 30° C.; Wavelength: 254 nm, R_{T}=7.493 min). ¹H NMR (400 MHz, CDCl₃) δ 8.76-8.74 (m, 1H), 8.58-8.53 (m, 1H), 8.01-7.98 (m, 1H), 7.76-7.68 (m, 1H), 7.43-7.37 (m, 1H), 6.20-6.06 (m, 1H), 5.80-5.50 (m, 1H), 5.08-5.04 (m, 1H), 4.79-4.66 (m, 2H), 4.48-4.34 (m, 1H), 3.71-3.58 (m, 1H), 3.21-2.99 (m, 2H), 2.75-2.61 (m, 1H), 1.67-1.60 (m, 3H), 1.54-1.53 (m, 6H), 1.34-1.26 (m, 6H). ¹⁹F NMR (376 MHz, CDCl₃) δ −64.70.

### 58B:

LC-MS (ESI): R_{T}=3.421 min, mass calcd. for C₂₈H₃₀ClF₃N₆O₃ 590.2, m/z found 591.1 [M+H]⁺. Chiral analysis (Column: Chiralpak IC 5 m 4.6*250 mm; Mobile Phase: ACN:DEA=100:0.2 at 1 mL/min; Temp: 30° C.; Wavelength: 254 nm, R_{T}=9.303 min). ¹H NMR (400 MHz, CDCl₃) δ 8.76-8.74 (m, 1H), 8.55-8.49 (m, 1H), 8.01-7.99 (m, 1H), 7.73-7.62 (m, 1H), 7.40-7.35 (m, 1H), 6.18-6.04 (m, 1H), 5.82-5.49 (m, 1H), 5.10-5.06 (m, 1H), 4.81-4.67 (m, 2H), 4.41-4.28 (m, 1H), 3.35-3.12 (m, 3H), 2.77-2.62 (m, 1H), 1.68-1.62 (m, 3H), 1.57-1.55 (m, 9H), 1.34-1.25 (m, 3H). ¹⁹F NMR (376 MHz, CDCl₃) δ −64.69.

### Compounds 59A and 59B

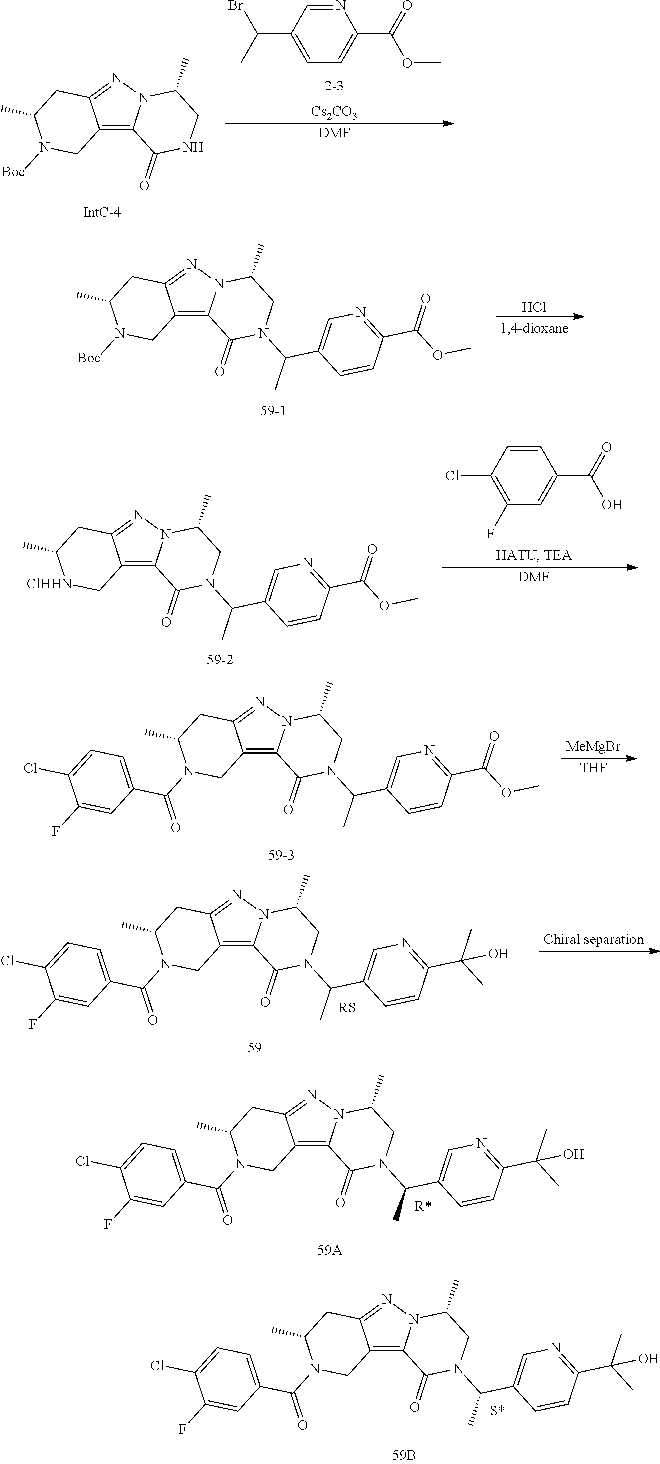

### Intermediate 59-1: tert-Butyl (3R,7R)-9-(1-(6-(methoxycarbonyl)pyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate

Methyl 5-(1-bromoethyl)picolinate 2-3 (850 mg, 90% purity, 3.13 mmol), tert-butyl (3R,7R)-3,7-dimethyl-10-oxo-3,4,7,8,9,10-hexahydropyrido[4′,3′:3,4]pyrazolo[1,5-a]pyrazine-2(1H)-carboxylate IntC-4 (940 mg, 90% purity, 2.64 mmol) and cesium carbonate (3.5 g, 10.7 mmol) were mixed in N,N-dimethylformamide (20 mL). After stirred at 45° C. for 3 hours under nitrogen atmosphere, the mixture was added water (100 mL) and extracted with ethyl acetate (80 mL). The combined organic layers were concentrated under reduced pressure and purified by silica gel column chromatography (dichloromethane:methanol=100:1 to 16:1) to give the title compound (1.3 g, 92% purity from LCMS, 94% yield) as white solids. LC-MS (ESI): R_{T}=2.46 min and 2.50 min, mass calcd. for C₂₅H₃₃N₅O₅ 483.3, m/z found 484.0 [M+H]⁺

### Intermediate 59-2

### Methyl 5-(1-((3R,7R)-3,7-dimethyl-10-oxo-1,3,4,7,8,10-hexahydropyrido[4′,3′:3,4]pyrazolo[1,5-a]pyrazin-9(2H)-yl)ethyl)picolinate hydrochloride salt

To a mixture of tert-butyl (3R,7R)-9-(1-(6-(methoxycarbonyl)pyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate 59-1 (1.3 g, 92% purity, 2.47 mmol) was added 4 M hydrochloride in dioxane (25 mL) at room temperature. After stirred at room temperature for 3 hours, the mixture was concentrated under reduced pressure to give the title product (1.1 g, 89% purity from LCMS, 94% yield) as white solids. LC-MS (ESI): R_{T}=1.15 min, mass calcd. for C₂₀H₂₅N₅O₃ 383.2, m/z found 384.1 [M+H]⁺

### Intermediate 59-3

### Methyl 5-(1-((3R,7R)-2-(4-chloro-3-fluorobenzoyl)-3,7-dimethyl-10-oxo-1,3,4,7,8,10-hexahydropyrido[4′,3′:3,4]pyrazolo[1,5-a]pyrazin-9(2H)-yl)ethyl)picolinate

To the solution of methyl 5-(1-((3R,7R)-3,7-dimethyl-10-oxo-1,3,4,7,8,10-hexahydropyrido[4′,3′:3,4]pyrazolo[1,5-a]pyrazin-9(2H)-yl)ethyl)picolinate hydrochloride 59-2 (500 mg, 89% purity, 1.06 mmol) and 2-(3H-[1,2,3]triazolo[4,5-b]pyridin-3-yl)-1,1,3,3-tetramethylisouronium (520 mg, 1.37 mmol) in N,N-dimethylformamide (8 mL) were added 4-chloro-3-fluorobenzoic acid (240 mg, 1.38 mmol) and triethylamine (0.6 mL, 4.64 mmol) at room temperature. After stirred at room temperature for 1 hour, the mixture was quenched with 0.5 M hydrochloride aqueous solution (15 mL) and extracted with ethyl acetate (15 mL) twice. The combined organic layers were washed with brine (20 mL), dried over Na₂SO₄₍ₛ₎ and filtered. The filtrate was concentrated and purified by C18 column (acetonitrile:water=60% to 70%) to give the title compound (500 mg, 83% purity, 72.5% yield) as white solids. LC-MS (ESI): R_{T}=1.50 min, mass calcd. for C₂₇H₂₇ClFN₅O₄ 539.2, m/z found 539.9 [M+H]⁺.

### Compound 59

### (3R,7R)-2-(4-chloro-3-fluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To the solution of methyl 5-(1-((3R,7R)-2-(4-chloro-3-fluorobenzoyl)-3,7-dimethyl-10-oxo-1,3,4,7,8,10-hexahydropyrido[4′,3′:3,4]pyrazolo[1,5-a]pyrazin-9(2H)-yl)ethyl)picolinate 59-3 (450 mg, 83% purity, 0.692 mmol) in tetrahydrofuran (5 mL) was added 1 M methylmagnesium bromide in tetrahydrofuran (7 mL, 7 mmol) at −70° C. under nitrogen atmosphere. After stirred at −70° C. for 3 hours, the mixture was quenched with ammonium chloride aqueous solution (5 mL) and extracted with ethyl acetate (10 mL) twice. The combined organic layers were washed with brine (15 mL), dried over Na₂SO₄₍ₛ₎ and filtered. The filtrate was concentrated and purified by C18 column (acetonitrile:water=45% to 60%) to give the title compound (300 mg, 97.4% purity, 78.2% yield) as yellow oil. LC-MS (ESI): R_{T}=2.52 min and 2.55 min, mass calcd. for C₂₈H₃₁ClFN₅O₃ 539.2, m/z found 540.0 [M+H]⁺.

### Compounds 59A and 59B

### (3R,7R)-2-(4-chloro-3-fluorobenzoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (59A) and (3R,7R)-2-(4-chloro-3-fluorobenzoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (59B)

The racemic (3R,7R)-2-(4-chloro-3-fluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo [1,5-a]pyrazin-10(7H)-one 59 (100 mg, 100% purity, 0.17 mmol) was separated by chiral Prep. (Column: Chiralpak IC 5 m 30*250 mm; Mobile Phase: MeOH:EtOH:DEA=50:50:0.2 at 60 mL/min; Temp: 30° C.; Wavelength: 254 nm) to give the compounds 59A (111.2 mg, 99.9% purity, 38% yield, 100% stereopure) as a white solid and 59B (92.7 mg, 99.8% purity, 31% yield, 99.9% stereopure) as a white solid.

### 59A:

LC-MS (ESI): R_{T}=3.219 min, mass calcd. for C₂₈H₃₁ClFN₅O₃ 539.2, m/z found 540.2 [M+H]⁺. Chiral analysis: Column: Chiralpak IC 5 m 4.6 mm*250 mm, Mobile phase: MeOH:EtOH:DEA=50:50:0.2 at 1 ml/min, Temp: 30° C.; Wavelength: 254 nm), R_{T}=8.907 min). ¹H NMR (400 MHz, DMSO-d₆) δ 8.50 (s, 1H), 7.82-7.74 (m, 1H), 7.72-7.69 (m, 1H), 7.67-7.61 (m, 1H), 7.56 (d, J=9.6 Hz, 1H), 7.31 (dd, J=8.4 and 1.6 Hz, 1H), 5.91-5.68 (m, 1H), 5.53-5.12 (m, 2H), 4.65-4.05 (m, 3H), 3.90-3.75 (m, 1H), 3.18-3.04 (m, 1H), 2.98-2.87 (m, 1H), 1.66-1.49 (m, 3H), 1.42 (s, 6H), 1.25 (d, J=6.4 Hz, 3H), 1.21-1.04 (m, 3H). ¹⁹F NMR (376 MHz, DMSO-d₆) δ −114.9, −115.1.

### 59B:

LC-MS (ESI): R_{T}=3.245 min, mass calcd. for C₂₈H₃₁ClFN₅O₃ 539.2, m/z found 540.2 [M+H]⁺. Chiral analysis: Column: Chiralpak IC 5 m 4.6 mm*250 mm, Mobile phase: MeOH:EtOH:DEA=50:50:0.2 at 1 ml/min, Temp: 30° C.; Wavelength: 254 nm), R_{T}=12.468 min). ¹H NMR (400 MHz, DMSO-d₆) δ 8.49 (s, 1H), 7.80-7.68 (m, 2H), 7.66-7.60 (m, 1H), 7.56 (d, J=10.4 Hz, 1H), 7.31 (dd, J=8.0 and 1.2 Hz, 1H), 5.93-5.67 (m, 1H), 5.52-5.12 (m, 2H), 4.62-4.05 (m, 3H), 3.57-3.38 (m, 2H), 2.99-2.87 (m, 1H), 1.66-1.50 (m, 3H), 1.47-1.38 (m, 9H), 1.25 (m, 3H). ¹⁹F NMR (376 MHz, DMSO-d₆) δ −114.9, −115.1.

### Compounds 60A and 60B

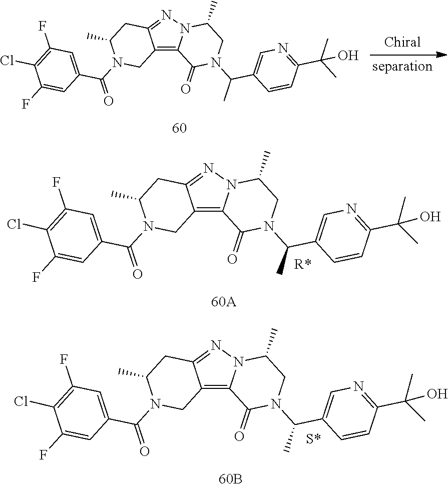 [SMILES: CC(c1ccc(C(C)(C)O)nc1)N1C[C@@H](C)n2nc3c(c2C1=O)CN(C(=O)c1cc(F)c(Cl)c(F)c1)[C@H](C)C3 ; C[C@@H]1Cc2nn3c(c2CN1C(=O)c1cc(F)c(Cl)c(F)c1)C(=O)N([C@H](C)c1ccc(C(C)(C)O)nc1)C[C@H]3C ; C[C@@H]1Cc2nn3c(c2CN1C(=O)c1cc(F)c(Cl)c(F)c1)C(=O)N([C@@H](C)c1ccc(C(C)(C)O)nc1)C[C@H]3C]

### Compound 60

### (3R,7R)-2-(4-chloro-3,5-difluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

Compound 60 (300 mg, 91% purity, 80.3% yield) was prepared following the same procedure using 4-chloro-3,5-difluorobenzoic acid as white solids. LC-MS (ESI): R_{T}=2.64 min and 2.66 min, mass calcd. for C₂₈H₃₀ClF₂N₅O₃ 557.2, m/z found 558.0 [M+H]⁺.

### Compounds 60A and 60B

### (3R,7R)-2-(4-chloro-3,5-difluorobenzoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (60A) and (3R,7R)-2-(4-chloro-3,5-difluorobenzoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (60B)

The racemate of (3R,7R)-2-(4-chloro-3,5-difluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one 60 (300 mg, 91% purity, 0.489 mmol) was separated by chiral Prep. HPLC (Column: Chiralpak IC 5 m 30*250 mm; Mobile Phase: methanol:ethanol:diethylamine=50:50:0.2 at 60 mL/min; Temp: 30° C.; Wavelength: 254 nm) to give the compounds 60A (83.8 mg, 99.5% purity, 30.5% yield, 100% stereopure) as a white solid and 60B (67.1 mg, 99.8% purity, 24.5% yield, 99.7% stereopure) as a white solid.

### 60A:

LC-MS (ESI): R_{T}=3.348 min, mass calcd. for C₂₈H₃₀ClF₂N₅O₃ 557.2, m/z found 558.2 [M+H]⁺. Chiral analysis (Column: Chiralpak IC, 5 m 4.6*250 mm; Mobile phase: MeOH:EtOH:DEA=50:50:0.2 at 1 ml/min; Temp: 30° C.; R_{T}=7.869 min). ¹H NMR (400 MHz, DMSO-d₆) δ 8.56-8.46 (m, 1H), 7.84-7.72 (m, 1H), 7.69-7.61 (m, 1H), 7.56-7.46 (m, 2H), 5.91-5.70 (m, 1H), 5.47-5.16 (m, 2H), 4.61-4.37 (m, 2H), 4.22-4.06 (m, 1H), 3.91-3.74 (m, 1H), 3.31-3.25 (m, 1H), 3.17-3.04 (m, 1H), 3.01-2.84 (m, 1H), 1.65-1.50 (m, 3H), 1.42 (s, 6H), 1.25 (d, J=6.0 Hz, 3H), 1.21-1.06 (m, 3H). ¹⁹F NMR (376 MHz, DMSO-d₆) δ −112.24.

### 60B:

LC-MS (ESI): R_{T}=3.373 min, mass calcd. for C₂₈H₃₀ClF₂N₅O₃ 557.2, m/z found 558.2 [M+H]⁺. Chiral analysis (Column: Chiralpak IC, 5 m 4.6*250 mm; Mobile phase: MeOH:EtOH:DEA=50:50:0.2 at 1 ml/min; Temp: 30° C.; R_{T}=10.195 min). ¹H NMR (400 MHz, DMSO-d₆) δ 8.54-8.40 (m, 1H), 7.82-7.70 (m, 1H), 7.68-7.58 (m, 1H), 7.56-7.44 (m, 2H), 5.93-5.69 (m, 1H), 5.52-5.15 (m, 2H), 4.59-4.06 (m, 3H), 3.57-3.38 (m, 2H), 3.31-3.25 (m, 1H), 3.03-2.84 (m, 1H), 1.65-1.51 (m, 3H), 1.48-1.34 (m, 9H), 1.25-1.05 (m, 3H). ¹⁹F NMR (376 MHz, DMSO-d₆) δ −112.22.

### Compounds 61A and 61B

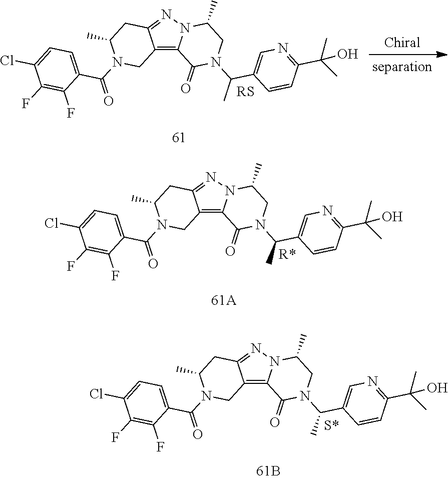 [SMILES: CC(c1ccc(C(C)(C)O)nc1)N1C[C@@H](C)n2nc3c(c2C1=O)CN(C(=O)c1ccc(Cl)c(F)c1F)[C@H](C)C3 ; C[C@@H]1Cc2nn3c(c2CN1C(=O)c1ccc(Cl)c(F)c1F)C(=O)N([C@H](C)c1ccc(C(C)(C)O)nc1)C[C@H]3C ; C[C@@H]1Cc2nn3c(c2CN1C(=O)c1ccc(Cl)c(F)c1F)C(=O)N([C@@H](C)c1ccc(C(C)(C)O)nc1)C[C@H]3C]

### Compound 61

### (3R,7R)-2-(4-chloro-2,3-difluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

Compound 61 (320 mg, 95% purity from LCMS, 76% yield) was prepared following the same procedure using 4-chloro-2,3-difluorobenzoic acid as white solids. LC-MS (ESI): R_{T}=1.45 min and 1.47 min, mass calcd. for C₂₈H₃₀ClF₂N₅O₃ 557.2, m/z found 558.0 [M+H]⁺.

### Compounds 61A and 61B

### (3R,7R)-2-(4-chloro-2,3-difluorobenzoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (61A) and (3R,7R)-2-(4-chloro-2,3-difluorobenzoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (61B)

The racemate of (3R,7R)-2-(4-chloro-2,3-difluorobenzoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one 61 (320 mg, 97% purity, 0.316 mmol) was separated by chiral Prep. HPLC (separation method: Column: Chiralpak IC, 5 m 30*250 mm; Mobile Phase: MeOH:EtOH:DEA=30:70:0.2 at 60 mL/min; Col. Temp: 30° C.; Wavelength: 254 nm) to give the title compounds 61A (99.4 mg, 99.4% purity, 33% yield, 100% stereopure) as a white solid and 61B (106.3 mg, 99.5% purity, 35% yield, 99.7% stereopure) as a white solid.

### 61A:

LC-MS (ESI): R_{T}=3.679 min, mass calcd. for C₂₈H₃₀ClF₂N₅O₃ 557.2, m/z found 558.2 [M+H]⁺. Chiral analysis (Column: Chiralpak IC, 5 m 4.6*250 mm; Mobile Phase: MeOH: EtOH:DEA=30:70:0.2 at 1 mL/min; Temp: 30° C.; Wavelength: 254 nm; R_{T}=6.426 min). ¹H NMR (400 MHz, CDCl₃) δ 8.55 (d, J=16.4 Hz, 1H), 7.74-7.68 (m, 1H), 7.41-7.37 (m, 1H), 7.29-7.22 (m, 1H), 7.10-7.07 (m, 1H), 6.20-6.03 (m, 1H), 5.89-5.48 (m, 1H), 4.79-4.54 (m, 2H), 4.46-4.30 (m, 1H), 4.23-4.15 (m, 1H), 3.71-3.60 (m, 1H), 3.12-2.99 (m, 2H), 2.70-2.63 (m, 1H), 1.63 (m, 3H), 1.53 (d, J=5.2 Hz, 6H), 1.33-1.27 (m, 6H). ¹⁹F NMR (376 MHz, CDCl₃) δ −136.27.

### 61B:

LC-MS (ESI): R_{T}=3.752 min, mass calcd. for C₂₈H₃₀ClF₂N₅O₃ 557.2, m/z found 558.2 [M+H]⁺. Chiral analysis (Column: Chiralpak IC, 5 m 4.6*250 mm; Mobile Phase: MeOH: EtOH:DEA=30:70:0.2 at 1 mL/min; Temp: 30° C.; Wavelength: 254 nm; R_{T}=8.214 min). ¹H NMR (400 MHz, CDCl₃) δ 8.52 (d, J=16.0 Hz, 1H), 7.71-7.64 (m, 1H), 7.40-7.36 (m, 1H), 7.31-7.23 (m, 1H), 7.12-7.09 (m, 1H), 6.20-6.01 (m, 1H), 5.90-5.49 (m, 1H), 4.81-4.54 (m, 2H), 4.38-4.15 (m, 2H), 3.37-3.25 (m, 2H), 3.10 (m, 1H), 2.72-2.64 (m, 1H), 1.65 (m, 3H), 1.57-1.54 (m, 9H), 1.33-1.18 (m, 3H). ¹⁹F NMR (376 MHz, CDCl₃) δ −136.28.

### Compounds 62A and 62B

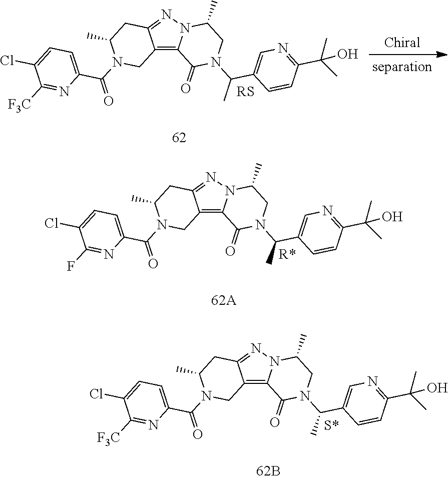 [SMILES: CC(c1ccc(C(C)(C)O)nc1)N1C[C@@H](C)n2nc3c(c2C1=O)CN(C(=O)c1ccc(Cl)c(C(F)(F)F)n1)[C@H](C)C3 ; C[C@@H]1Cc2nn3c(c2CN1C(=O)c1ccc(Cl)c(F)n1)C(=O)N([C@H](C)c1ccc(C(C)(C)O)nc1)C[C@H]3C ; C[C@@H]1Cc2nn3c(c2CN1C(=O)c1ccc(Cl)c(C(F)(F)F)n1)C(=O)N([C@@H](C)c1ccc(C(C)(C)O)nc1)C[C@H]3C]

### Compound 62

### (3R,7R)-2-(5-chloro-6-(trifluoromethyl)picolinoyl)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

Compound 62 (160 mg, 85% purity from LCMS, 47% yield) was prepared following the same procedure using 5-chloro-6-(trifluoromethyl)picolinic acid as white solids. LC-MS (ESI): R_{T}=1.51 min and 1.54 min, mass calcd. for C₂₈H₃₀ClF₃N₆O₃ 590.2, m/z found 591.0 [M+H]⁺.

### Compounds 62A and 62B

### (3R,7R)-2-(5-chloro-6-(trifluoromethyl)picolinoyl)-9-((R*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (62A) and (3R,7R)-2-(5-chloro-6-(trifluoromethyl)picolinoyl)-9-((S*)-1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one (62B)

The racemic (3R,7R)-2-(5-chloro-6-(trifluoromethyl)picolinoyl)-9-(1-(6-(2-hydroxy propan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one 62 (160 mg, 85% purity, 0.230 mmol) was separated by chiral Prep.HPLC (separation method: Column: Chiralpak IC, μm 30*250 mm; Mobile Phase: ACN: IPA:DEA=90:10:0.2 at 60 mL/min; Col. Temp: 30° C.; Wavelength: 254 nm) to give the title compounds 62A (50.3 mg, 99.9% purity, 37% yield, 100% stereopure) as a white solid and 62B (47.2 mg, 98.0% purity, 34% yield, 99.9% stereopure) as a white solid.

### 62A:

LC-MS (ESI): R_{T}=2.932 min, mass calcd. for C₂₈H₃₀ClF₃N₆O₃ 590.2, m/z found 591.2 [M+H]⁺. Chiral analysis (Column: Chiralpak IC, 5 m 4.6*250 mm; Mobile Phase: ACN:IPA:DEA=90:10:0.2 at 1 mL/min; Temp: 30° C.; Wavelength: 254 nm; R_{T}=5.251 min). ¹H NMR (400 MHz, CDCl₃) δ 8.61-8.51 (m, 1H), 8.01-7.8 (m, 2H), 7.77-7.66 (m, 1H), 7.44-7.35 (m, 1H), 6.24-6.04 (m, 1H), 5.82-5.05 (m, 1H), 4.85-4.60 (m, 2H), 4.49-4.33 (m, 2H), 3.74-3.57 (m, 1H), 3.31-3.08 (m, 1H), 3.07-2.94 (m, 1H), 2.78-2.59 (m, 1H), 1.67-1.60 (m, 3H), 1.55-1.52 (m, 6H), 1.40-1.23 (m, 6H). ¹⁹F NMR (376 MHz, CDCl₃) δ −66.15.

### 62B:

LC-MS (ESI): R_{T}=2.978 min, mass calcd. for C₂₈H₃₀ClF₃N₆O₃ 590.2, m/z found 591.2 [M+H]⁺. Chiral analysis (Column: Chiralpak IC, 5 m 4.6*250 mm; Mobile Phase: ACN:IPA:DEA=90:10:0.2 at 1 mL/min; Temp: 30° C.; Wavelength: 254 nm; R_{T}=6.710 min). ¹H NMR (400 MHz, CDCl₃) δ 8.61-8.39 (m, 1H), 8.12-7.79 (m, 2H), 7.76-7.59 (m, 1H), 7.45-7.31 (m, 1H), 6.29-5.99 (m, 1H), 5.85-5.10 (m, 1H), 4.89-4.57 (m, 2H), 4.43-4.21 (m, 2H), 3.44-3.06 (m, 3H), 2.81-2.56 (m, 1H), 1.69-1.61 (m, 3H), 1.57-1.53 (m, 9H), 1.37-1.26 (m, 3H). ¹⁹F NMR (376 MHz, CDCl₃) δ −66.16.

### Compounds 63A and 63B

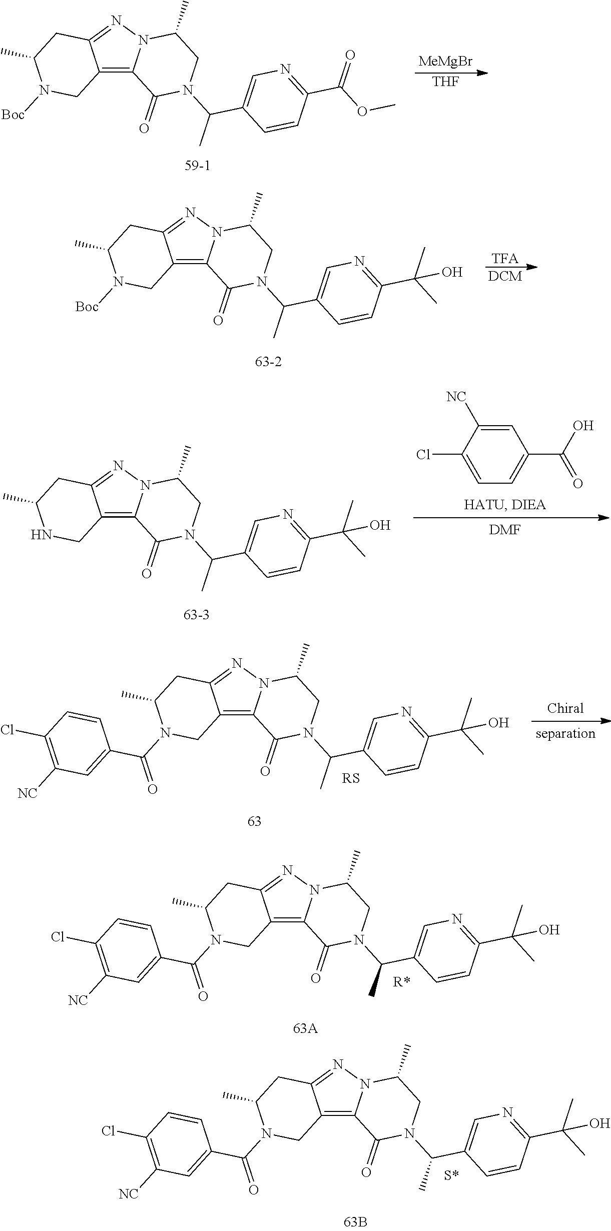

### Intermediate 63-2

### tert-Butyl (3R,7R)-9-(1-(6-(2-hydroxypropan-2-yl)pyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate

To a solution of tert-butyl (3R,7R)-9-(1-(6-(methoxycarbonyl)pyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate 59-1 (1.00 g, 90% purity, 1.86 mmol) in tetrahydrofuran (10 mL) was added 3 M methylmagnesium bromide in tetrahydrofuran (6 mL, 18 mmol) at −78° C. After stirred at temperature below −40° C. for 1 hour, the mixture was quenched with ammonium chloride aqueous solution (10 mL) at −78° C. and extracted with ethyl acetate (10 mL) twice. The combined organic layers were washed with brine (10 mL), dried over Na₂SO₄₍ₛ₎ and concentrated to give a residue, which was purified by silica gel column chromatography (dichloromethane:methanol=100:1 to 40:1) to give the title compound (780 mg, 90% purity from ¹H NMR, 78% yield) as white solids. LC-MS (ESI): R_{T}=1.53 min, mass calcd. for C₂₆H₃₇N₅O₄ 483.3, m/z found 484.2 [M+H]⁺.

### Intermediate 63-3

### (3R,7R)-9-(1-(6-(2-hydroxypropan-2-yl)pyridin-3-yl)ethyl)-3,7-dimethyl-1,2,3,4,8,9-hexahydropyrido[4′,3′:3,4]pyrazolo[1,5-a]pyrazin-10(7H)-one

To a solution of tert-butyl (3R,7R)-9-(1-(6-(2-hydroxypropan-2-yl)pyridin-3-yl)ethyl)-3,7-dimethyl-10-oxo-3,4,7,8,9,10-hexahydropyrido[4′,3′:3,4]pyrazolo[1,5-a]pyrazine-2(1H)-carboxylate 63-2 (100 mg, 90% purity, 0.186 mmol) in dichloromethane (1 mL) were added 2,2,2-trifluoroacetic acid (0.5 mL) at 0° C. After stirred at room temperature for 1 hour under nitrogen atmosphere, the reaction mixture was quenched with saturated sodium bicarbonate aqueous solution (10 mL) at 0° C. and concentrated under reduced pressure to give the title compound (380 mg, 18% purity from LCMS, 96% yield) as yellow solids. LC-MS (ESI): R_{T}=1.23 min, mass calcd. for C₂₁H₂₉N₅O₂383.2, m/z found 384.1 [M+H]⁺.

### Compound 63

### 2-Chloro-5-((3R,7R)-9-(1-(6-(2-hydroxypropan-2-yl)pyridin-3-yl)ethyl)-3,7-di methyl-10-oxo-1,2,3,4,7,8,9,10-octahydropyrido[4′,3′:3,4]pyrazolo [1,5-a]pyrazine-2-carbonyl)benzonitrile

To a solution of 4-chloro-3-cyanobenzoic acid (39 mg, 0.215 mmol) in N,N-dimethylformamide (4 mL) were added N-ethyl-N-isopropylpropan-2-amine (69 mg, 0.534 mmol), 2-(3H-[1,2,3]triazolo[4,5-b]pyridin-3-yl)-1,1,3,3-tetramethyluronium hexafluorophosphate (V) (102 mg, 0.268 mmol) and (3R,7R)-9-(1-(6-(2-hydroxypropan-2-yl)pyridin-3-yl)ethyl)-3,7-dimethyl-1,2,3,4,8,9-hexahydropyrido [4′,3′:3,4]pyrazolo[1,5-a]pyrazin-10(7H)-one 63-3 (380 mg, 18% purity, 0.178 mmol) at room temperature under nitrogen atmosphere. After stirred at room temperature overnight, the mixture was diluted with water (20 mL) and extracted with ethyl acetate (20 mL) three times. The combined organic layers were washed with water (20 mL), dried over Na₂SO₄₍ₛ₎ and filtered. The filtrate was concentrated and purified by C18 column (acetonitrile:water=05% to 95%) to give the title compound (70 mg, 52% purity from LCMS, 37% yield) as yellow solids. LC-MS (ESI): R_{T}=1.41 min, mass calcd. for C₂₉H₃₁ClN₆O₃ 546.2, m/z found 547.1 [M+H]⁺.

### Compounds 63A and 63B

### 2-chloro-5-((3R,7R)-9-((R*)-1-(6-(2-hydroxypropan-2-yl)pyridin-3-yl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile (63A) and 2-chloro-5-((3R,7R)-9-((S*)-1-(6-(2-hydroxypropan-2-yl)pyridin-3-yl)ethyl)-3,7-dimethyl-10-oxo-1,2,3,4,7,8,9,10-octahydropyrido[4′,3′:3,4]pyrazolo[1,5-a]pyrazine-2-carbonyl)benzonitrile (63B)

A racemic mixture of 2-chloro-5-((3R,7R)-9-(1-(6-(2-hydroxypropan-2-yl)pyridin-3-yl)ethyl)-3,7-dimethyl-10-oxo-1,2,3,4,7,8,9,10-octahydropyrido[4′,3′:3,4]pyrazolo [1,5-a]pyrazine-2-carbonyl)benzonitrile 63 (70 mg, 52% purity, 0.067 mmol) was separated by chiral prep. HPLC (Column: Chiralpak IJ 5 m 30*250 mm; Mobile Phase: CO₂:MeOH:DEA=85:15:0.3 at 60 g/min; Temp: 40° C.; Wavelength: 254 nm; Back pressure: 100 bar) to afford the title compounds 63A (20 mg, 99.5% purity from LCMS, 55% yield, 100% steropure) as a white solid and 63B (13 mg, 98.4% purity from LCMS, 35% yield, 99.7% steropure) as a white solid.

### 63A:

LC-MS (ESI): R_{T}=3.042 min, mass calcd. for C₂₉H₃₁ClN₆O₃ 546.2, m/z found 547.3 [M+H]⁺. Chiral HPLC (Column: Chiralpak IJ 5 m 30*250 mm; Mobile Phase: CO₂: MeOH:DEA=85:15:0.3 at 3 g/min; Temp: 40° C.; Wavelength: 254 nm; Back pressure: 100 bar, R_{T}=6.26 min). ¹H NMR (400 MHz, DMSO-d₆) δ 8.53-8.46 (m, 1H), 8.18-8.07 (m, 1H), 7.87-7.75 (m, 3H), 7.68-7.61 (m, 1H), 5.91-5.70 (m, 1H), 5.54-5.39 (m, 1H), 5.28-5.14 (m, 1H), 4.54-4.37 (m, 2H), 4.19-4.08 (m, 1H), 3.89-3.76 (m, 1H), 3.29 (br s, 1H), 3.16-3.06 (m, 1H), 3.00-2.88 (m, 1H), 1.66-1.51 (m, 3H), 1.42 (s, 6H), 1.26 (d, J=6.4 Hz, 3H), 1.20-1.06 (m, 3H).

### 63B:

LC-MS (ESI): R_{T}=3.072 min, mass calcd. for C₂₉H₃₁ClN₆O₃ 546.2, m/z found 547.3 [M+H]⁺. Chiral HPLC (Column: Chiralpak IJ 5 m 30*250 mm; Mobile Phase: CO₂: MeOH:DEA=85:15:0.3 at 3 g/min; Temp: 40° C.; Wavelength: 254 nm; Back pressure: 100 bar, R_{T}=7.73 min). ¹H NMR (400 MHz, DMSO-d₆) δ 8.53-8.43 (m, 1H), 8.18-8.08 (m, 1H), 7.87-7.73 (m, 3H), 7.66-7.59 (m, 1H), 5.91-5.71 (m, 1H), 5.51-5.40 (m, 1H), 5.27-5.13 (m, 1H), 4.59-4.35 (m, 2H), 4.21-4.07 (m, 1H), 3.54-3.42 (m, 2H), 3.29 (br s, 1H), 3.01-2.89 (m, 1H), 1.63-1.52 (m, 3H), 1.47-1.37 (m, 9H), 1.21-1.09 (m, 3H).

### Compounds 64A and 64B

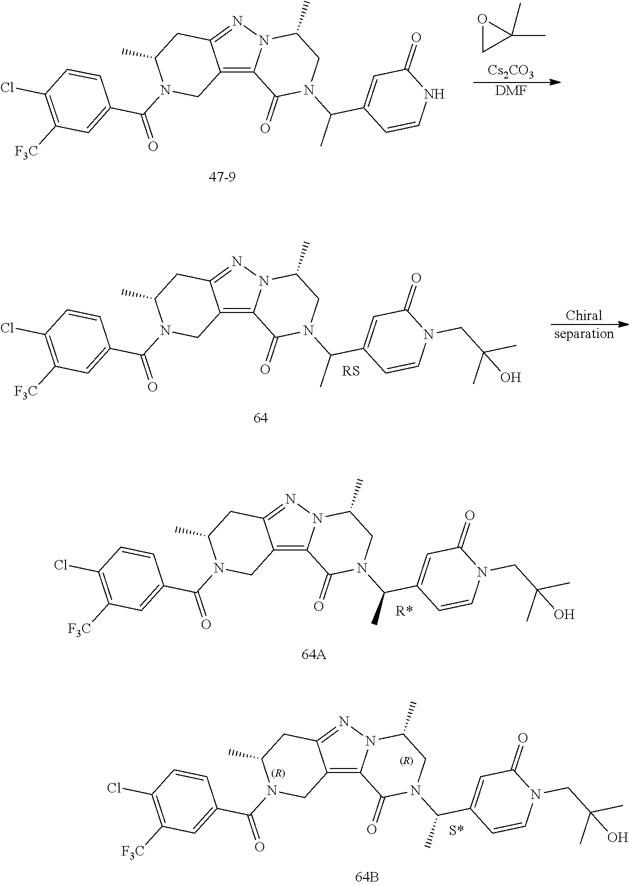

### Compound 64

### (3R,7R)-2-(4-chloro-3-(trifluoromethyl)benzoyl)-9-(1-(1-(2-hydroxy-2-methylpropyl)-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethyl)benzoyl)-3,7-dimethyl-9-(1-(2-oxo-1,2-dihydropyridin-4-yl)ethyl)-1,2,3,4,8,9-hexahydropyrido[4′,3′:3,4]pyrazolo[1,5-a]pyrazin-10(7H)-one 47-9 (160 mg, 57% purity, 0.166 mmol) in N,N-dimethylformamide (3 mL) was added cesium carbonate (500 mg, 1.53 mmol) and 2,2-dimethyloxirane (35 mg, 0.485 mmol) at room temperature. After stirred 8 hours at 120° C., the reaction mixture was quenched with water (10 mL) and extracted with ethyl acetate (10 mL) twice. The combined organic layers were washed with brine (15 mL), dried over Na₂SO₄₍ₛ₎ and filtered. The filtrate was concentrated and purified by C18 column (acetonitrile:water=45% to 55%) to give the title compound (110 mg, 86% purity, 91.7% yield) as yellow oil. LC-MS (ESI): R_{T}=1.44 min, mass calcd. for C₃₀H₃₃ClF₃N₅O₄ 619.2, m/z found 620.1 [M+H]⁺.

### Compounds 64A and 64B

### (3R,7R)-2-(4-chloro-3-(trifluoromethyl)benzoyl)-9-((R*)-1-(1-(2-hydroxy-2-methylpropyl)-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (64A) and (3R,7R)-2-(4-chloro-3-(trifluoromethyl)benzoyl)-9-((S*)-1-(1-(2-hydroxy-2-methylpropyl)-2-oxo-1,2-dihydropyridin-4-yl)ethyl)-3,7-dimethyl-1,2,3,4,8,9-hexahydropyrido[4′,3′:3,4]pyrazolo[1,5-a]pyrazin-10(7H)-one (64B)

The racemate of (3R,7R)-2-(4-chloro-3-(trifluoromethyl)benzoyl)-9-(1-(1-isopropyl-6-oxo-1,6-dihydropyridazin-4-yl)ethyl)-3,7-dimethyl-1,2,3,4,8,9-hexahydropyrido[4′,3′:3,4]pyrazolo[1,5-a]pyrazin-10(7H)-one 64 (110 mg, 86% purity, 0.253 mmol) was separated by chiral Prep. (Column: Chiralpak IB N-5 5 m 30*250 mm; Mobile Phase: ACN:IPA=90:10 at 60 mL/min; Temp: 30° C.; Wavelength: 254 nm) to give the title compounds 64A (21.2 mg, 99.5% purity, 22.2% yield, 99.8% stereopure) and 64B (39.9 mg, 99.0% purity, 24.6% yield, 99.4% stereopure) as white solids.

### 64A:

LC-MS (ESI): R_{T}=3.240 min, mass calcd. for C₃₀H₃₃ClF₃N₅O₄ 619.2, m/z found 620.2 [M+H]⁺. Chiral HPLC (Column: Chiralpak IB N-5 5 m 4.6*250 mm; Mobile Phase: ACN:IPA=90:10 at 1 mL/min; Temp: 30° C.; Wavelength: 254 nm, R_{T}=4.713 min). ¹H NMR (400 MHz, CDCl₃) δ 7.78 (s, 1H), 7.59-7.52 (m, 2H), 7.30-7.24 (m, 1H), 6.60 (s, 1H), 6.26-6.13 (m, 1H), 5.94-5.35 (m, 2H), 4.88-4.24 (m, 3H), 4.06-3.97 (m, 2H), 3.92-3.82 (m, 1H), 3.69-3.56 (m, 1H), 3.14-3.09 (m, 2H), 2.76-2.62 (m, 1H), 1.49 (d, J=6.0 Hz, 3H), 1.40 (d, J=6.8 Hz, 3H), 1.33-1.21 (m, 9H). ¹⁹F NMR (376 MHz, CDCl₃) δ −62.81.

### 64B:

LC-MS (ESI): R_{T}=3.849 min, mass calcd. for C₃₀H₃₃ClF₃N₅O₄ 619.2, m/z found 620.2 [M+H]⁺. Chiral HPLC (Column: Chiralpak IB N-5 5 m 4.6*250 mm; Mobile Phase: ACN:IPA=90:10 at 1 mL/min; Temp: 30° C.; Wavelength: 254 nm, R_{T}=5.374 min). ¹H NMR (400 MHz, CDCl₃) δ 7.78 (s, 1H), 7.59-7.53 (m, 2H), 7.31-7.22 (m, 1H), 6.58 (s, 1H), 6.26-6.05 (m, 1H), 5.96-5.36 (m, 2H), 4.93-4.21 (m, 3H), 4.06-3.83 (m, 3H), 3.44-3.29 (m, 2H), 3.22-2.91 (m, 1H), 2.77-2.62 (m, 1H), 1.57 (d, J=6.8 Hz, 3H), 1.50 (d, J=6.4 Hz, 3H), 1.34-1.20 (m, 9H). ¹⁹F NMR (376 MHz, CDCl₃) δ −62.78.

### Compounds 65A and 65B

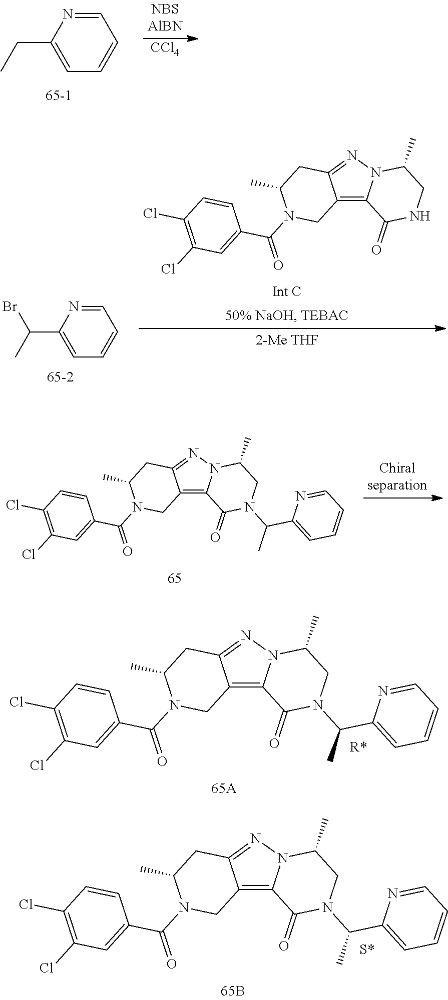

### Intermediate 65-2

### 2-(1-Bromoethyl)pyridine

To a solution of 2-ethylpyridine 65-1 (2.0 g, 18.7 mmol) in carbon tetrachloride (50 mL) were added N-bromosuccinimide (3.7 g, 20.8 mmol) and 2,2′-azobis(2-methylpropionitrile) (306 mg, 1.86 mmol). After stirred at 90° C. for 1 hour, the reaction mixture was filtered. The filtrate washed with brine (100 mL) twice, dried over Na₂SO₄₍ₛ₎ and filtered. The filtrate was concentrated and purified by silica gel column chromatography (petroleum ether:ethyl acetate=100:1) to give the title compound (3.4 g, 90% purity from ¹H NMR, 88% yield) as red oil. ¹H NMR (400 MHz, CDCl₃) δ 8.58-8.57 (m, 1H), 7.71-7.66 (m, 1H), 7.45-7.43 (m, 1H), 7.21-7.18 (m, 1H), 5.23 (q, J=7.2 Hz, 1H), 2.07 (d, J=7.2 Hz, 3H).

### Compound 65

### (3R,7R)-2-(3,4-Dichlorobenzoyl)-3,7-dimethyl-9-(1-(pyridin-2-yl)ethyl)-1,2,3,4,8,9-hexahydropyrido[4′,3′:3,4]pyrazolo[1,5-a]pyrazin-10(7H)-one

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (300 mg, 90% purity, 0.687 mmol) and 2-(1-bromoethyl)pyridine 65-2 (213 mg, 90% purity, 1.03 mmol) in 2-methyltetrahydrofuran (3 mL) was added 50% wt. sodium hydroxide in water (3 mL) and benzyltriethylammonium chloride (50 mg, 0.220 mmol) slowly at room temperature. After stirred at room temperature for 5 hours, the mixture was added water (80 mL) and extracted with ethyl acetate (80 mL) for three times. The combined organic layers were washed with brine (80 mL) and concentrated to give a residue, which was purified by silica gel column chromatography (petroleum ether:ethyl acetate=1:1) to give the title compound (350 mg, 97% purity from LCMS, 99% yield) as white solids. LC-MS (ESI): R_{T}=1.55 min, mass calcd. for C₂₅H₂₅Cl₂N₅O₂ 497.1, m/z found 498.0 [M+H]⁺.

### Compounds 65A and 65B

### (3R,7R)-2-(3,4-dichlorobenzoyl)-3,7-dimethyl-9-((R*)-1-(pyridin-2-yl)ethyl)-1,2,3,4,8,9-hexahydropyrido[4′,3′:3,4]pyrazolo[1,5-a]pyrazin-10(7H)-one (65A) and (3R,7R)-2-(3,4-dichlorobenzoyl)-3,7-dimethyl-9-((S*)-1-(pyridin-2-yl)ethyl)-1,2,3,4,8,9-hexahydropyrido[4′,3′:3,4]pyrazolo[1,5-a]pyrazin-10(7H)-one (65B)

The racemic mixture of (3R,7R)-2-(3,4-dichlorobenzoyl)-3,7-dimethyl-9-(1-(pyridin-2-yl)ethyl)-1,2,3,4,8,9-hexahydropyrido[4′,3′:3,4]pyrazolo[1,5-a]pyrazin-10(7H)-one 65 (400 mg, 97% purity, 0.778 mmol) was separated by chiral Prep. HPLC separation condition: (Column: Chiralpak IC 5 m 30*250 mm; Mobile Phase: ACN:IPA=90:10 at 25 mL/min; Temp: 30° C.; Wavelength: 254 nm) to give the title compounds 65A (152 mg, 99.7% purity, 39% yield, 100% steropure) and 65B (150 mg, 99.6% purity, 38% yield, 99.9% steropure) as white solids.

### 65A:

LC-MS (ESI): R_{T}=3.354 min, mass calcd. for C₂₅H₂₅Cl₂N₅O₂ 497.1, m/z found 498.1 [M+H]⁺. Chiral HPLC (Column: Column: Chiralpak IC 5 m 4.6*250 mm; Mobile Phase: ACN:IPA=90:10 at 1 mL/min; Temp: 30° C.; Wavelength: 254 nm, R_{T}=7.724 min). ¹H NMR (400 MHz, DMSO-d₆) δ 8.61-8.52 (m, 1H), 7.85-7.73 (m, 3H), 7.46-7.28 (m, 3H), 5.94-5.69 (m, 1H), 5.52-5.12 (m, 1H), 4.61-4.36 (m, 2H), 4.25-4.04 (m, 1H), 3.89-3.74 (m, 1H), 3.30-3.19 (m, 1H), 2.99-2.85 (m, 1H), 2.69-2.53 (m, 1H), 1.67-1.45 (m, 3H), 1.22-1.04 (m, 6H).

### 65B:

LC-MS (ESI): R_{T}=3.331 min, mass calcd. for C₂₅H₂₅Cl₂N₅O₂ 497.1, m/z found 498.1 [M+H]⁺. Chiral HPLC (Column: Column: Chiralpak IC 5 m 4.6*250 mm; Mobile Phase: ACN:IPA=90:10 at 1 mL/min; Temp: 30° C.; Wavelength: 254 nm, R_{T}=9.092 min). ¹H NMR (400 MHz, DMSO-d₆) δ 8.59-8.52 (m, 1H), 7.84-7.73 (m, 3H), 7.45-7.27 (m, 3H), 5.94-5.70 (m, 1H), 5.51-5.14 (m, 1H), 4.61-4.33 (m, 2H), 4.25-4.06 (m, 1H), 3.65-3.44 (m, 2H), 2.99-2.87 (m, 1H), 2.71-2.55 (m, 1H), 1.65-1.50 (m, 3H), 1.44 (d, J=6.8 Hz, 3H), 1.27-1.06 (m, 3H).

### Compounds 66A and 66B

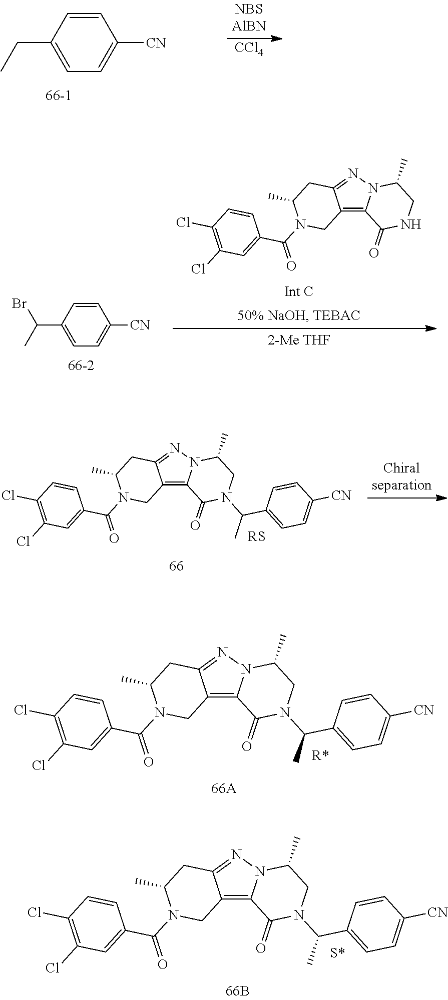

### Intermediate 66-2

### 4-(1-bromoethyl)benzonitrile

To a solution of 4-ethylbenzonitrile 66-1 (2.0 g, 18.7 mmol) in carbon tetrachloride (50 mL) was added N-bromosuccinimide (3.7 g, 20.8 mmol) and 2,2′-azobis(2-methylpropionitrile) (306 mg, 1.86 mmol). After stirred at 90° C. for 1 hour, the reaction mixture was filtered. The filtrate washed with brine (100 mL) twice, dried over Na₂SO₄₍ₛ₎ and filtered. The filtrate was concentrated and purified by silica gel column chromatography (petroleum ether:ethyl acetate=100:1) to give the title compound (3.4 g, 63% purity from ¹H NMR, 62% yield) as red oil.

### Compound 66

### 4-(1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)benzonitrile

To a solution of (3R,7R)-2-(3,4-dichlorobenzoyl)-3,7-dimethyl-1,2,3,4,8,9-hexahydropyrido[4′,3′:3,4]pyrazolo[1,5-a]pyrazin-10(7H)-one Int C (200 mg, 90% purity, 0.422 mmol), 4-(1-bromoethyl)benzonitrile 66-2 (210 mg, 63% purity, 0.499 mmol) and N-benzyl-N,N-diethylethanaminium chloride (15 mg, 0.066 mmol) in 2-methyltetrahydrofuran (4 mL) was added 50% wt. sodium hydroxide solution (1 mL) at 0° C. After stirred at room temperature for 8 hours, the reaction mixture was quenched with water (10 mL) and extracted with ethyl acetate (15 mL) twice. The combined organic layers were dried over Na₂SO₄₍ₛ₎ and filtered. The filtrate was concentrated under reduced pressure to give a crude, which was purified by silica gel column chromatography (dichloromethane:methanol=1:0 to 20:1) to afford the title compound (200 mg, 77% purity from LCMS, 45% of yield) as white solids. LC-MS (ESI): R_{T}=2.33 min, mass calcd. for C₂₇H₂₅Cl₂N₅O₂ 521.1, m/z found 522.1 [M+H]⁺.

### Compounds 66A and 66B

### 4-((R*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)benzonitrile (66A) and 4-((S*)-1-((3R,7R)-2-(3,4-dichlorobenzoyl)-3,7-dimethyl-10-oxo-1,3,4,7,8,10-hexahydropyrido[4′,3′:3,4]pyrazolo[1,5-a]pyrazin-9(2H)-yl)ethyl)benzonitrile (66B)

The racemic mixture of (3R,7R)-2-(4-chloro-3-(trifluoromethyl)benzoyl)-3,7-dimethyl-9-(1-(2-((S)-2-methylmorpholino)pyrimidin-5-yl)ethyl)-1,2,3,4,8,9-hexahydropyrido[4′,3′:3,4]pyrazolo[1,5-a]pyrazin-10(7H)-one 66 (200 mg, 77% purity, 0.241 mmol) was separated by chiral HPLC (Column: Chiralpak IG 5 μm 20*250 mm; Mobile Phase: CO₂: IPA=50:50 at 25 mL/min; Col. Temp: 40° C.; Wavelength: 230 nm, Back pressure: 97 bar) to afford the title compounds 66A (75 mg, 38% yield, 99.9% purity from LCMS, 100% stereopure) and 66B (65 mg, 33% yield, 99.2% purity from LCMS, 100% stereopure) as white solids.

### 66A:

LC-MS (ESI): R_{T}=3.781 min, mass calcd. for C₂₇H₂₅Cl₂N₅O₂ 521.1, m/z found 522.0 [M+H]⁺. Chiral HPLC (Column: Chiralpak IG 5 μm 20*250 mm; Mobile Phase: CO₂: IPA=50:50 at 25 mL/min; Col. Temp: 40° C.; Wavelength: 230 nm, Back pressure: 97 bar, R_{T}=7.24 min). ¹H NMR (400 MHz, DMSO-d₆) δ 7.89-7.82 (m, 2H), 7.78-7.72 (m, 2H), 7.64-7.52 (m, 2H), 7.45-7.43 (m, 1H), 5.94-5.72 (m, 1H), 5.50-5.17 (m, 1H), 4.62-4.36 (m, 2H), 4.27-4.06 (m, 1H), 3.84-3.71 (m, 1H), 3.08 (br, s, 1H), 2.99-2.86 (m, 1H), 2.63-2.54 (m, 1H), 1.66-1.45 (m, 3H), 1.30-1.05 (m, 6H).

### 66B:

LC-MS (ESI): R_{T}=3.800 min, mass calcd. for C₂₇H₂₅Cl₂N₅O₂ 521.1, m/z found 522.0 [M+H]⁺. Chiral HPLC (Column: Chiralpak IG 5 μm 20*250 mm; Mobile Phase: CO₂: IPA=50:50 at 25 mL/min; Col. Temp: 40° C.; Wavelength: 230 nm, Back pressure: 97 bar, R_{T}=11.64 min). ¹H NMR (400 MHz, DMSO-d₆) δ 7.88-7.71 (m, 4H), 7.62-7.50 (m, 2H), 7.47-7.40 (m, 1H), 6.09-5.69 (m, 1H), 5.55-5.17 (m, 1H), 4.60-4.04 (m, 3H), 3.52-3.37 (m, 2H), 2.99-2.85 (m, 1H), 2.45-2.37 (m, 1H), 1.62-1.51 (m, 3H), 1.44 (d, J=6.4 Hz, 3H), 1.23-1.08 (m, 3H).

### Compounds 67A and 67B

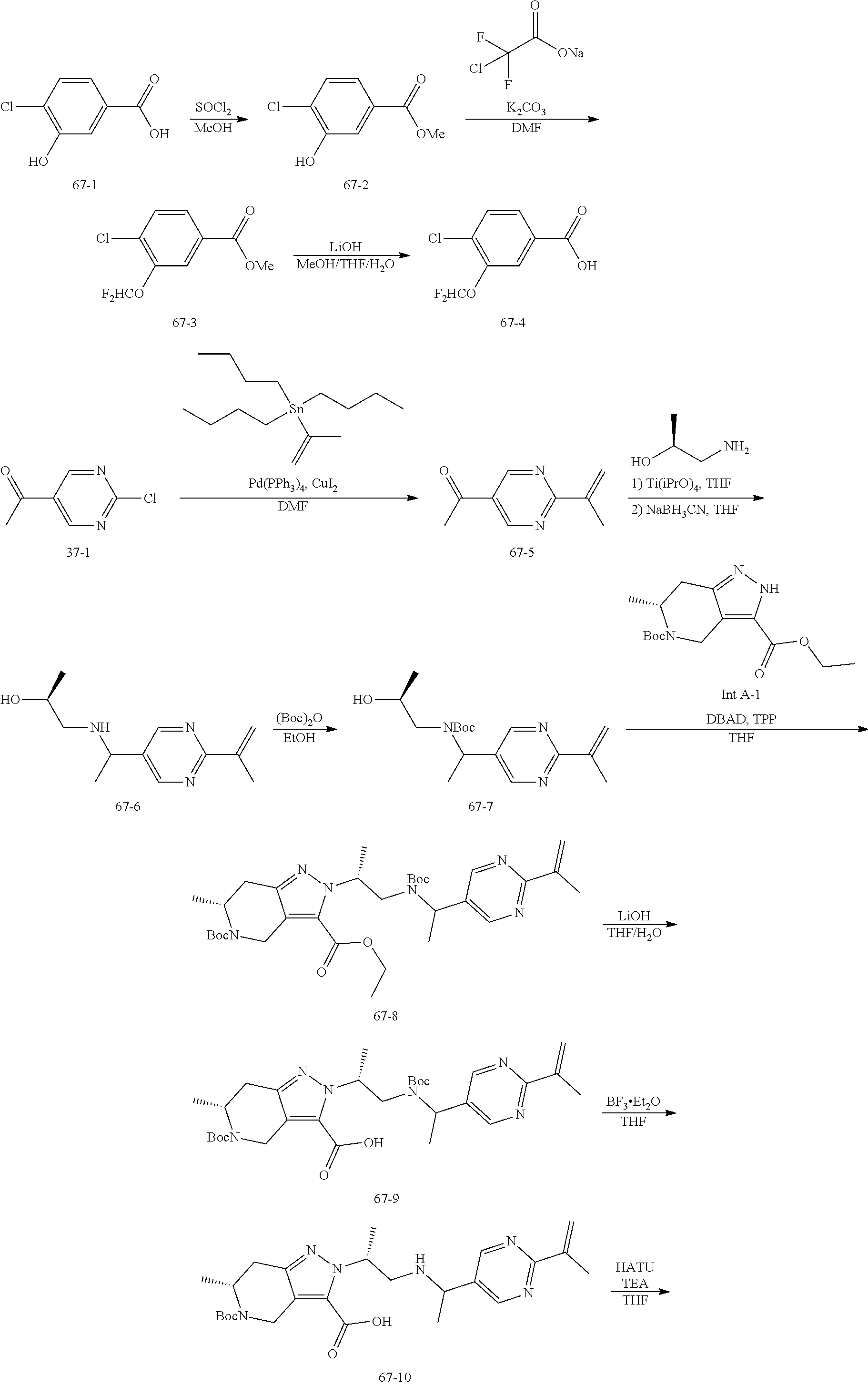

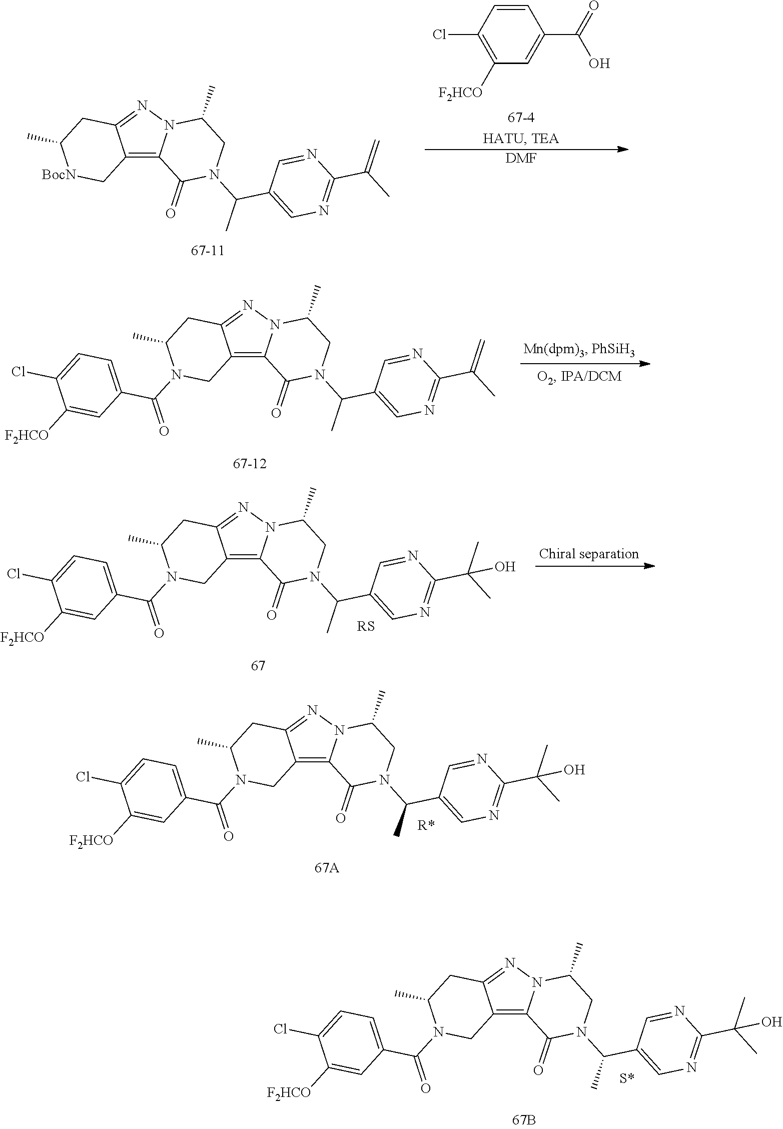

### Intermediate 67-2

### Methyl 4-chloro-3-hydroxybenzoate

To a solution of 4-chloro-3-hydroxybenzoic acid 67-1 (3.0 g, 17.4 mmol) in methanol (30 mL) was added sulfurous dichloride (2.48 g, 20.9 mmol) dropwise at 0° C. The resulting mixture was stirred at 0° C. for 10 minutes, then heated to 60° C. and stirred for 2 hours. After cooled to the room temperature, the reaction mixture was concentrated to afford a residue, which was dissolved in ethyl acetate (50 mL), washed with 5% wt. sodium bicarbonate aqueous solution (20 mL), brine (20 mL), dried over Na₂SO₄₍ₛ₎ and filtered. The filtrate was concentrated in vacuo to give the title product (3.20 g, 96.4% purity from LCMS, 95% yield) as yellow solids. LC-MS (ESI): R_{T}=1.391 min, mass calcd. for C₈H₇ClO₃ 186.0 m/z found 373.1 [2M+H]⁺.

### Intermediate 67-3

### Methyl 4-chloro-3-(difluoromethoxy)benzoate

To a solution of methyl 4-chloro-3-hydroxybenzoate 67-2 (3.20 g, 17.2 mmol) and potassium carbonate (2.84 g, 20.6 mmol) in N,N-dimethylformamide (50 mL) was added sodium 2-chloro-2,2-difluoroacetate (3.14 g, 20.6 mmol). The resulting mixture was stirred at 80° C. overnight. After cooled down to room temperature, the reaction mixture was poured into water (200 mL), extracted with ethyl acetate (100 mL) for three times. The combined organic layers were washed with brine (100 mL), dried over Na₂SO₄₍ₛ₎ and filtered. The filtrate was concentrated in vacuo to afford a residue, which was purified by column chromatography on silica gel (petroleum ether:ethyl acetate=8:1) to give the title product (2.0 g, 90% purity from ¹HNMR, 44% yield) as yellow solids. ¹H NMR (400 MHz, CDCl₃) δ 7.89 (s, 1H), 7.85 (dd, J=8.4 and 1.6 Hz, 1H), 7.53 (d, J=8.4 Hz, 1H), 6.59 (t, J=72.8 Hz, 1H), 3.93 (s, 3H).

### Intermediate 67-4

### 4-Chloro-3-(difluoromethoxy)benzoic acid

To a solution of methyl 4-chloro-3-(difluoromethoxy)benzoate 46-3 (2.0 g, 90% purity, 7.61 mmol) in methanol (5 mL) and tetrahydrofuran (15 mL) was added a solution of lithium hydroxide monohydrate (638.5 mg, 15.2 mmol) in water (10 mL) at 20° C. The resulting mixture was stirred at 20° C. for 2 hours, then poured into water (50 mL) and extracted with ethyl acetate (20 mL) twice. The aqueous layer was acidified by 10% wt. hydrochloride aqueous solution to pH=4 and extracted with dichloromethane (20 mL) for three times. The combined dichloromethane organic layers were washed with brine (10 mL), dried over Na₂SO₄₍ₛ₎ and filtered. The filtrate was concentrated in vacuo to give the title product (1.85 g, 90% purity from ¹HNMR, 98.3% yield) as yellow solids. ¹H NMR (400 MHz, CDCl₃) δ 7.97 (s, 1H), 7.93 (dd, J=8.4 and 1.6 Hz, 1H), 7.58 (d, J=8.4 Hz, 1H), 6.61 (t, J=72.8 Hz, 1H).

### Intermediate 67-5

### 1-(2-(Prop-1-en-2-yl)pyrimidin-5-yl)ethan-1-one

To a solution of 1-(2-chloropyrimidin-5-yl)ethan-1-one 37-1 (1.0 g, 6.39 mmol) and tributyl(prop-1-en-2-yl)stannane (3.2 g, 9.66 mmol) in N,N-dimethylformamide (10 mL) under nitrogen atmosphere were added tetrakis(triphenylphosphine)palladium (500 mg, 0.43 mmol) and copper iodide (500 mg, 1.58 mmol). After stirred at 120° C. for 3 hours, the reaction mixture was diluted with ethyl acetate (30 mL) and treated with potassium fluoride aqueous solution (30 mL). After stirred vigorously for 1 hour, the mixture was filtered with kieselguhr. The filtrate was diluted with ethyl acetate (30 mL), washed with saturated sodium bicarbonate solution (30 mL), brine (30 mL), dried over Na₂SO₄₍ₛ₎, filtered and concentrated under reduced pressure to give the residue, which was purified by gel column chromatography (petroleum ether:ethyl acetate=5:1) to give the title compound (600 mg, 95% purity from ¹H NMR, 55.0% yield) as white solids. LC-MS (ESI): R_{T}=1.348 min, mass calcd. for C₉H₁₀N₂O 162.1 m/z found 163.0 [M+H]⁺. ¹H NMR (400 MHz, CDCl₃) δ 9.19 (s, 2H), 6.61 (s, 1H), 5.69 (s, 1H), 2.63 (s, 3H), 2.28 (s, 3H).

### Intermediate 67-6

### (2S)-1-((1-(2-(Prop-1-en-2-yl)pyrimidin-5-yl)ethyl)amino)propan-2-ol

To a mixture of 1-(2-(prop-1-en-2-yl)pyrimidin-5-yl)ethan-1-one 67-5 (250 mg, 100% purity, 1.54 mmol) in tetrahydrofuran (5 mL) were added (S)-1-aminopropan-2-ol (116 mg, 1.54 mmol) and tetraisopropoxytitanium (1.4 ml, 4.73 mmol). The reaction mixture was stirred at room temperature overnight. After cooling to 0° C., sodium cyanoborohydride (77 mg, 1.88 mmol) and acetic acid (0.1 ml, 1.75 mmol) was added. The resulting mixture was stirred at 25° C. for 2 hours. The reaction was quenched with water (10 ml), then adjusted with sodium bicarbonate(s) to pH 8˜9 and filtered. The filtrate was extracted with dichloromethane and methanol (40 mL, 10:1) twice. The combined organic layers were dried over Na₂SO₄₍ₛ₎ and filtered. The filtrate was concentrated under reduced pressure to give the crude (190 mg, 87% purity by LCMS, 48% yield) as white solids. LC-MS (ESI): R_{T}=1.68 min, mass calcd. for C₁₂H₁₉N₃O 221.2 m/z found 222.0 [M+H]⁺.

### Intermediate 67-7

### Tert-butyl ((S)-2-Hydroxypropyl)(1-(2-(prop-1-en-2-yl)pyrimidin-5-yl)ethyl)carbamate

To a solution of (2S)-1-((1-(2-(prop-1-en-2-yl)pyrimidin-5-yl)ethyl)amino)propan-2-ol 67-6 (295 mg, 87% purity, 1.16 mmol) in ethanol (6 ml) was added di-tert-butyl dicarbonate (300 mg, 1.38 mmol) at 0° C. After stirred at 30° C. for 16 hours, the solvent was removed in vacuo to give the title compound (380 mg, 95% purity from LCMS, 97% yield) as colorless oil. LC-MS (ESI): R_{T}=1.554 min, mass calcd. for C₁₇H₂₇N₃O₃ 321.2 m/z found 322.0 [M+H]⁺.

### Intermediate 67-8

### 5-(tert-Butyl) 3-ethyl (6R)-2-((2R)-1-((tert-butoxycarbonyl)(1-(2-(prop-1-en-2-yl)pyrimidin-5-yl)ethyl)amino)propan-2-yl)-6-methyl-2,4,6,7-tetrahydro-5H-pyrazolo[4,3-c]pyridine-3,5-dicarboxylate

To a solution of tert-butyl ((S)-2-hydroxypropyl)(1-(2-(prop-1-en-2-yl)pyrimidin-5-yl)ethyl)carbamate 67-7 (400 mg, 95% purity, 1.18 mmol) and 5-(tert-butyl) 3-ethyl (R)-6-methyl-2,4,6,7-tetrahydro-5H-pyrazolo[4,3-c]pyridine-3,5-dicarboxylate IntA-1 (438 mg, 1.42 mmol) in dry tetrahydrofuran (10 mL) were added triphenylphosphine (617 mg, 2.36 mmol) and (E)-di-tert-butyl diazene-1,2-dicarboxylate (543 mg, 2.36 mmol) at 0° C. under nitrogen atmosphere. After stirred at 25° C. overnight, the reaction was poured into water (30 mL), extracted with ethyl acetate (30 mL) for three times. The combined organic layers were washed with brine (30 mL), dried over Na₂SO₄₍ₛ₎ and filtered. The filtrate was concentrated in vacuo. The residue was purified by silica gel column chromatography (petroleum ether:ethyl acetate=1:1) to give the title product (650 mg, 96% purity from LCMS, 78% yield) as yellow solids. LC-MS (ESI): R_{T}=1.971 min, mass calcd. for C₃₂H₄₈N₆O₆ 612.4 m/z found 613.4 [M+H]⁺.

### Intermediate 67-9

### (6R)-5-(tert-Butoxycarbonyl)-2-((2R)-1-((tert-butoxycarbonyl)(1-(2-(prop-1-en-2-yl)pyrimidin-5-yl)ethyl)amino)propan-2-yl)-6-methyl-4,5,6,7-tetrahydro-2H-pyrazolo[4,3-c]pyridine-3-carboxylic acid

To a solution of 5-(tert-Butyl) 3-ethyl (6R)-2-((2R)-1-((tert-butoxycarbonyl)(1-(2-(prop-1-en-2-yl)pyrimidin-5-yl)ethyl)amino)propan-2-yl)-6-methyl-2,4,6,7-tetrahydro-5H-pyrazolo[4,3-c]pyridine-3,5-dicarboxylate 67-8 (550 mg, 96% purity, 0.862 mmol) in tetrahydrofuran (8 mL) was added lithium hydroxide monohydrate (72 mg, 1.72 mmol) in water (2 mL). After stirred at 40° C. overnight, the mixture was diluted by water (15 mL), and then removed the volatile, acidized with 1 M hydrochloride aqueous solution to pH=4˜5, extracted with ethyl acetate (30 mL) for three times. The organic layers were combined, washed with brine (20 mL), dried over Na₂SO₄₍ₛ₎ and filtered. The filtrate was concentrated to give the title compound (520 mg, 90% purity from LCMS, 93% yield) as white solids. LC-MS (ESI): R_{T}=1.434 min, mass calcd. for C₃₀H₄₄N₆O₆ 584.3 m/z found 585.4 [M+H]⁺.

### Intermediate 67-10

### (6R)-6-Methyl-2-((2R)-1-((1-(2-(prop-1-en-2-yl)pyrimidin-5-yl)ethyl)amino) propan-2-yl)-4,5,6,7-tetrahydro-2H-pyrazolo[4,3-c]pyridine-3-carboxylic acid

To a solution of (6R)-5-(tert-Butoxycarbonyl)-2-((2R)-1-((tert-butoxycarbonyl)(1-(2-(prop-1-en-2-yl)pyrimidin-5-yl)ethyl)amino)propan-2-yl)-6-methyl-4,5,6,7-tetrahydro-2H-pyrazolo[4,3-c]pyridine-3-carboxylic acid 67-9 (520 mg, 90% purity, 0.800 mmol) in tetrahydrofuran (8 mL) was added boron trifluoride diethyl etherate (1 mL, 48% purity, 4.0 mmol) at 0° C. After stirred at room temperature for 6 hours, the mixture was concentrated and gave the crude product (342 mg, 94.7% purity, 100% yield) as white solids, which was used directly in the following step. LC-MS (ESI): R_{T}=1.098 min, mass calcd. for C₂₀H₂₈N₆O₂ 384.2 m/z found 385.3 [M+H]⁺.

### Intermediate 67-11

### (3R,7R)-3,7-Dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one

To a solution of (6R)-6-methyl-2-((2R)-1-((1-(2-(prop-1-en-2-yl)pyrimidin-5-yl)ethyl)amino)propan-2-yl)-4,5,6,7-tetrahydro-2H-pyrazolo[4,3-c]pyridine-3-carboxylic acid 67-10 (340 mg, 94.7% purity, 0.80 mmol) in N,N-dimethylformamide (4 mL) was added triethylamine (0.4 ml, 2.67 mmol) and 2-(3H-[1,2,3]triazolo[4,5-b]pyridin-3-yl)-1,1,3,3-tetramethyluronium hexafluorophosphate(V) (676 mg, 1.78 mmol) at room temperature. After stirred at room temperature for 1 hour, the mixture was quenched with water (10 mL), extracted with ethyl acetate (10 mL) for three times. The combined organic phases were washed by brine (10 ml), dried over Na₂SO₄₍ₛ₎, and concentrated in vacuo. The residue was purified by C18 column chromatography (acetonitrile:water (0.1% ammonium bicarbonate)=20 to 30%) to give the title compound (200 mg, 92.5% purity from LCMS, 63% yield) as white solids. LC-MS (ESI): R_{T}=1.09 min, mass calcd. for C₂₀H₂₆N₆O 366.2 m/z found 367.3 [M+H]⁺.

### Intermediate 67-12

### (3R,7R)-2-(4-Chloro-3-(difluoromethoxy)benzoyl)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one

To a solution of rac-(3R,7R)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one 67-11 (100 mg, 92.5% purity, 0.253 mmol), 4-chloro-3-(difluoromethoxy)benzoic acid 67-4 (73 mg, 90% purity, 0.27 mmol) in N,N-dimethylformamide (2 mL) was added 2-(3H-[1,2,3]triazolo[4,5-b]pyridin-3-yl)-1,1,3,3-tetramethylisouronium hexafluorophosphate(V) (121 mg, 0.32 mmol) at 0° C. After stirred at 0° C. for 10 minutes, triethylamine (75 mg, 0.74 mmol) was added dropwise to the reaction at 0° C. Then the mixture was stirred at 0° C. for 3 hours. The reaction mixture was quenched with water (10 mL), extracted with acetate (10 mL) for three times, dried over Na₂SO₄₍ₛ₎, filtered. The filtrate was concentrated to get a residue, which was purified by C18 chromatography (acetonitrile:water (+0.02% ammonium bicarbonate)=40-60%) to give the title compound (120 mg, 88.8% purity from LCMS, 76% yield) as yellow solids. LC-MS (ESI): R_{T}=1.653 min, mass calcd. for C₂₈H₂₉ClF₂N₆O₃ 570.2 m/z found 571.1 [M+H]⁺.

### Compound 67

### (3R,7R)-2-(4-Chloro-3-(difluoromethoxy)benzoyl)-9-(1-(2-(2-hydroxypropan-2-yl)pyrimidin-5-yl)ethyl)-3,7-dimethyl-1,2,3,4,8,9-hexahydropyrido [4′,3′:3,4]pyrazolo[1,5-a]pyrazin-10(7H)-one

To a solution of (3R,7R)-2-(4-chloro-3-(difluoromethoxy)benzoyl)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one 67-12 (120.0 mg, 88.8% purity, 0.187 mmol) in isopropyl alcohol (2.0 mL) and dichloromethane (0.1 mL) was added tris(((Z)-2,2,6,6-tetramethyl-5-oxohept-3-en-3-yl)oxy)manganese (17 mg, 0.03 mmol) and (phenyl)silane (57 mg, 0.53 mmol) at 0° C. The resulting mixture was stirred at 0° C. under a balloon oxygen atmosphere for 2 hours. The reaction mixture was purified by column chromatography on silica gel (petroleum ether:ethyl acetate from 1:1 to 0:1) and further purified by C18 chromatography (acetonitrile:water (+0.02% ammonium bicarbonate)=40-60%) to give the title compound (58.7 mg, 99.0% purity from LCMS, 52.9% yield) as white solids. LC-MS (ESI): R_{T}=2.938 min, mass calcd. for C₂₈H₃₁ClF₂N₆O₄ 588.2, m/z found 589.1 [M+H]⁺.

### Compounds 67A and 67B

### (3R,7R)-2-(4-Chloro-3-(difluoromethoxy)benzoyl)-9-((R*)-1-(2-(2-hydroxy propan-2-yl)pyrimidin-5-yl)ethyl)-3,7-dimethyl-1,2,3,4,8,9-hexahydropyrido [4′,3′:3,4]pyrazolo[1,5-a]pyrazin-10(7H)-one (67A) and (3R,7R)-2-(4-chloro-3-(difluoromethoxy)benzoyl)-9-((S*)-1-(2-(2-hydroxy propan-2-yl)pyrimidin-5-yl)ethyl)-3,7-dimethyl-1,2,3,4,8,9-hexahydropyrido [4′,3′:3,4]pyrazolo[1,5-a]pyrazin-10(7H)-one (67B)

The racemate of (3R,7R)-2-(4-chloro-3-(difluoromethoxy)benzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one 67 (58.7 mg, 99.0% purity, 0.099 mmol) was separated with chiral HPLC (Column: Chiral ART Cellulose-SC 10 m 50*250 mm; Mobile Phase: Methanol/Ethanol/DEA=50/50/0.2 at 100 mL/min; Col. Temp: 25° C.; Wavelength: 254 nm) to afford the title compounds 67a (23 mg, 39% yield, 97.6% purity from LCMS, 98.1% stereopure) as white solids and 67B (19 mg, 32% yield, 97.0% purity from LCMS, 99.2% stereopure) as white solids.

### 67A:

LC-MS (ESI): R_{T}=2.949 min, mass calcd. for C₂₈H₃₁ClF₂N₆O₄ 588.2, m/z found 589.1 [M+H]⁺. Chiral analysis (Column: Chiral ART Cellulose-SC 10 m 4.6*250 mm; Mobile Phase: Methanol/Ethanol/DEA=50/50/0.2 at 1 mL/min; Col. Temp: 25° C.; Wavelength: 254 nm, R_{T}=8.796 min). ¹H NMR (400 MHz, CDCl₃) δ 8.75 (s, 2H), 7.53-7.51 (m, 1H), 7.31 (s, 1H), 7.27-7.25 (m, 1H), 6.57 (t, J=72.8 Hz, 1H), 6.11 (br s, 1H), 5.79-5.11 (m, 1H), 4.89-4.22 (m, 4H), 3.72-3.65 (m, 1H), 3.08-3.03 (m, 2H), 2.73-2.60 (m, 1H), 1.67 (d, J=6.4 Hz, 3H), 1.59-1.58 (m, 6H), 1.37 (d, J=6.4 Hz, 3H), 1.32-1.22 (m, 3H). ¹⁹F NMR (376 MHz, CDCl₃) δ −81.18-−81.71.

### 67B:

LC-MS (ESI): R_{T}=2.927 min, mass calcd. for C₂₈H₃₁ClF₂N₆O₄ 588.2, m/z found 589.1 [M+H]⁺. Chiral analysis (Column: Chiral ART Cellulose-SC 10 m 4.6*250 mm; Mobile Phase: Methanol/Ethanol/DEA=50/50/0.2 at 1 mL/min; Col. Temp: 25° C.; Wavelength: 254 nm, R_{T}=6.105 min). ¹H NMR (400 MHz, CDCl₃) δ 8.71 (s, 2H), 7.52 (d, J=8.0 Hz, 1H), 7.32 (s, 1H), 7.27-7.25 (m, 1H), 6.57 (t, J=72.8 Hz, 1H), 6.10 (br s, 1H), 5.73-5.17 (m, 1H), 4.85-4.51 (m, 2H), 4.42-4.28 (m, 2H), 3.42-3.32 (m, 2H), 3.01 (br s, 1H), 2.74-2.62 (m, 1H), 1.67 (d, J=6.8 Hz, 3H), 1.58 (s, 9H), 1.33-1.21 (m, 3H). ¹⁹F NMR (376 MHz, CDCl₃) δ −81.12-−81.77.

### Compounds 68A and 68B

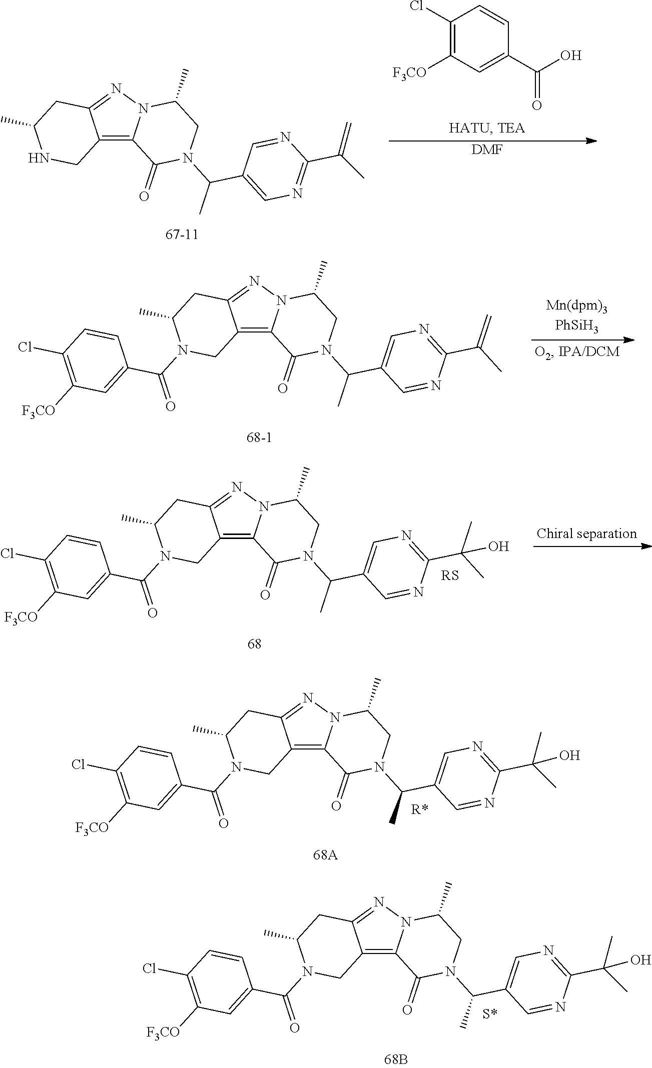

### Intermediate 68-1

### (3R,7R)-2-(4-Chloro-3-(trifluoromethoxy)benzoyl)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one

To a solution of (3R,7R)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one 67-11 (100 mg, 90% purity, 0.25 mmol), 4-chloro-3-(trifluoromethoxy)benzoic acid (71 mg, 0.30 mmol) in N,N-dimethylformamide (2 mL) was added 2-(3H-[1,2,3]triazolo[4,5-b]pyridin-3-yl)-1,1,3,3-tetramethylisouronium hexafluorophosphate(V) (121 mg, 0.32 mmol). After stirred at 0° C. for 10 minutes, triethylamine (75 mg, 0.74 mmol) was added dropwise at 0° C. Then stirring was continued at 0° C. for 3 hours. The reaction mixture was quenched with brine (10 mL), extracted with acetate (10 mL) for three times, dried over Na₂SO₄₍ₛ₎, filtered. The filtrate was concentrated to get a residue, which was purified by C18 chromatography (acetonitrile:water (+0.02% ammonium bicarbonate)=40-60%) to give the title (130 mg, 92.7% purity from LCMS, 83.3% yield) as yellow solids. LC-MS (ESI): R_{T}=2.480 min, mass calcd. for C₂₈H₂₈ClF₃N₆O₃ 588.2 m/z found 589.1 [M+H]⁺.

### Compound 68

### (3R,7R)-2-(4-Chloro-3-(trifluoromethoxy)benzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one

To a solution of (3R,7R)-2-(4-chloro-3-(trifluoromethoxy)benzoyl)-3,7-dimethyl-9-(1-(2-(prop-1-en-2-yl)pyrimidin-5-yl)ethyl)-1,2,3,4,8,9-hexahydropyrido[4′,3′:3,4]pyrazolo[1,5-a]pyrazin-10(7H)-one 68-1 (130.0 mg, 92.7% purity, 0.205 mmol) in isopropyl alcohol (2.0 mL) and dichloromethane (0.1 mL) was added tris(((Z)-2,2,6,6-tetramethyl-5-oxohept-3-en-3-yl)oxy)manganese (19 mg, 0.03 mmol) and phenylsilane (62 mg, 0.57 mmol) at 0° C. The resulting mixture was stirred at 0° C. under a balloon oxygen atmosphere for 2 hours. The reaction mixture was purified by column chromatography on silica gel (petroleum ether:ethyl acetate from 1:1 to 0:1) and further purified by C18 chromatography (acetonitrile:water (+0.02% ammonium bicarbonate)=40-60%) to give the title compound (57.4 mg, 94.7% purity from LCMS, 43.8% yield) as white solids. LC-MS (ESI): R_{T}=3.190 min, mass calcd. for C₂₈H₃₀ClF₃N₆O₄ 606.2 m/z found 607.1 [M+H]⁺.

### Compounds 68A and 68B

### (3R,7R)-2-(4-Chloro-3-(trifluoromethoxy)benzoyl)-9-((R*)-1-(2-(2-hydroxy propan-2-yl)pyrimidin-5-yl)ethyl)-3,7-dimethyl-1,2,3,4,8,9-hexahydropyrido [4′,3′:3,4]pyrazolo[1,5-a]pyrazin-10(7H)-one (68A) and (3R,7R)-2-(4-chloro-3-(trifluoromethoxy)benzoyl)-9-((S*)-1-(2-(2-hydroxy propan-2-yl)pyrimidin-5-yl)ethyl)-3,7-dimethyl-1,2,3,4,8,9-hexahydropyrido [4′,3′:3,4]pyrazolo[1,5-a]pyrazin-10(7H)-one (68B)

The racemate of (3R,7R)-2-(4-chloro-3-(trifluoromethoxy)benzoyl)-9-(1-(2-(2-hydroxypropan-2-yl)pyrimidin-5-yl)ethyl)-3,7-dimethyl-1,2,3,4,8,9-hexahydropyrido[4′,3′:3,4]pyrazolo[1,5-a]pyrazin-10(7H)-one 68 (57.4 mg, 94.7% purity, 0.087 mmol) was separated with chiral HPLC (Column: DAICEL IG 5 μm 20*250 mm; Mobile Phase: ACN=100 at 100 mL/min; Col. Temp: 40° C.; Wavelength: 214 nm) to afford the title compounds 68A (12 mg, 20% yield, 96.9% purity from SFC, 96.9% stereopure) as white solids and 68B (15 mg, 26% yield, 97.6% purity from LCMS, 100% stereopure) as white solids.

### 68A:

LC-MS (ESI): R_{T}=2.877 min, mass calcd. for C₂₈H₃₀ClF₃N₆O₄ 606.2, m/z found 607.4 [M+H]⁺. Chiral analysis (Column: DAICEL IG 5 m 4.6*250 mm; Mobile Phase: ACN=100 at 0.5 mL/min; Col. Temp: 40° C.; Wavelength: 214 nm, R_{T}=12.445 min). ¹H NMR (400 MHz, CDCl₃) δ 8.79 (s, 2H), 7.55 (d, J=8.0 Hz, 1H), 7.41 (s, 1H), 7.33 (dd, J=8.0 and 1.2 Hz, 1H), 6.13 (br s, 1H), 5.73-5.43 (m, 1H), 4.98-4.14 (m, 4H), 3.70 (s, 1H), 3.07-2.95 (m, 2H), 2.69-2.65 (m, 1H), 1.68 (s, 3H), 1.62 (s, 6H), 1.39 (d, J=4.4 Hz, 3H), 1.25 (s, 3H). ¹⁹F NMR (376 MHz, CDCl₃) δ −57.75.

### 68B:

LC-MS (ESI): R_{T}=2.851 min, mass calcd. for C₂₈H₃₀ClF₃N₆O₄ 606.2, m/z found 607.4 [M+H]⁺. Chiral analysis (Column: DAICEL IG 5 m 4.6*250 mm; Mobile Phase: ACN=100 at 0.5 mL/min; Col. Temp: 40° C.; Wavelength: 214 nm, R_{T}=16.843 min). ¹H NMR (400 MHz, CDCl₃) δ 8.78 (s, 2H), 7.55 (d, J=8.4 Hz, 1H), 7.42 (s, 1H), 7.33 (d, J=8.0 Hz, 1H), 6.09 (br s, 1H), 5.72-5.40 (m, 1H), 4.98-4.21 (m, 4H), 3.41 (s, 2H), 3.01-2.69 (m, 2H), 1.70 (s, 3H), 1.63 (s, 9H), 1.25 (s, 3H). ¹⁹F NMR (376 MHz, CDCl₃) δ −57.72.

### BIOLOGICAL EXAMPLES

### Anti-HBV Activity of Compounds of Formula (I)

### Procedure

The anti HBV activity was measured using the HepG2.117 cell line, a stable, inducibly HBV producing cell line, which replicates HBV in the absence of doxycycline (Tet-off system). The HepG2 cell line is available from ATCCR under number HB-8065. Transfection of the HepG2 cell line can be as described in Sun and Nassal 2006 Journal of Hepatology 45 (2006) 636-645 “Stable HepG2- and Huh7-based human hepatoma cell lines for efficient regulated expression of infectious hepatitis B virus”.

For the antiviral assay, HBV replication was induced, followed by a treatment with serially diluted compound in 96-well plates. After 3 days of treatment, the antiviral activity was determined by quantification of intracellular HBV DNA using real-time PCR and an HBV specific primer set and probe.

| | Forward Primer |
|---|---|
| SEQ ID. No: 1 | |
| | GTGTCTGCGGCGTTTTATCA |
| | Reverse Primer |
| SEQ ID. No: 2 | |
| | GACAAACGGGCAACATACCTT |
| | Taqman Probe |
| SEQ ID. No: 3 | |
| | CCTCTKCATCCTGCTGCTATGCCTCATC_FAM-BHQ1 |

Cytotoxicity of the compounds was tested using HepG2 or HepG2.117 cells, incubated for 3 or 4 days in the presence of compounds. The viability of the cells was assessed using the PERKIN ELMER ATPlite Luminescence Assay System.”

Human Liver Microsome Stability t½ (Min) of Compounds of Formula (I)

### Procedure

Pooled human liver microsomes (0.5 mg/mL) was pre-incubated with 1 μM test compounds or control compounds (verapamil, warfarin and cerivastatin) in 0.1M phosphate buffer pH 7.4 containing 1 mM MgCl₂ at 37° C. The final concentration of DMSO and acetonitrile are 0.05% and 0.2%, respectively. All incubations are performed singularly for each compound. NADPH was added to initiate the reaction with the final concentration of 1 mM and the final incubation volume of 400 μL.

At 6 time points (0, 5, 10, 20, 40 and 60 min) reactions were stopped by the removal of 50 μL of the incubation mixture into plates containing acetonitrile at a of 1:3 (v:v). The plates were centrifuged at 3000 rpm for 10 min at 4° C. to precipitate the protein. Following protein precipitation, the sample supernatants were combined in cassettes of up to 8 compounds. The internal standard was added (1:1 supernatant to internal standard solution) and samples were analyzed using standard LC-MS conditions.

From a plot of ln peak area ratio (compound peak area/internal standard peak area) against time, the gradient of the line is determined. Subsequently, half-life is calculated using the equations below:

Elimination rate constant (k)=(−gradient)

Half-life (t½) (min)=0.693/k

### Results

TABLE 4

| | EC50 | CC50 | HLM Metabolism |
|---|---|---|---|
| Compound | (μM, mean | (μM, mean | t_{1/2} |
| Number | value) | value) | (min) |
| 1C | 0.044 | >50.0 | >180 |
| 1D | 0.021 | >50.0 | >180 |
| 2A | 0.099 | >50.0 | >180 |
| 2B | 0.015 | >50.0 | 138 |
| 3A | 0.068 | 33.7 | >180 |
| 3B | 0.023 | 16.4 | 133 |
| 4A | 0.28 | >50 | >180 |
| 4B | 0.057 | >50.0 | >180 |
| 5A | 0.087 | >50 | >180 |
| 5B | 0.017 | 28.4 | 125 |
| 6A | 0.086 | >50 | 70.7 |
| 6B | 0.065 | >50.0 | >180 |
| 7A | 0.52 | 44.8 | >180 |
| 7B | 0.048 | 16.9 | >180 |
| 8A | 0.024 | 20.1 | >180 |
| 8B | 0.009 | 18.7 | >180 |
| 9A | 0.022 | >50 | 50.4 |
| 9B | 0.02 | >50.0 | 110 |
| 10B | 0.087 | >50.0 | >180 |
| 11A | 0.039 | >50.0 | >180 |
| 11B | 0.032 | >50.0 | >180 |
| 12A | 0.6 | >50.0 | >180 |
| 12B | 0.068 | >50.0 | >180 |
| 1A | 0.21 | >50.0 | 14.1 |
| 1B | 1.49 | >50.0 | 36.7 |
| 13A | 0.037 | >50 | 113 |
| 13B | 0.091 | >50 | 129 |
| 14A | 0.012 | >50 | 82 |
| 14B | 0.015 | >50 | 70.1 |
| 15A | 0.063 | >50 | >180 |
| 15B | 0.019 | >50 | >180 |
| 16A | 0.061 | >50 | >180 |
| 16B | 0.059 | >50 | >180 |
| 17A | 0.077 | >50 | 174 |
| 17B | 0.026 | >50 | 95.7 |
| 18A | 0.084 | >50 | >180 |
| 18B | 0.031 | >50 | >180 |
| 18C | 0.03 | >50 | >180 |
| 18D | 0.024 | >50 | >180 |
| 19A | 0.026 | >50 | >180 |
| 19B | 0.015 | >50 | >180 |
| 19C | 0.011 | >50 | >180 |
| 19D | 0.011 | >50 | >180 |
| 20A | 0.041 | >50 | >180 |
| 20B | 0.089 | >50 | 156 |
| 20C | 0.016 | >50 | 61.5 |
| 20D | 0.032 | >50 | 88.1 |
| 21A | 0.035 | >50 | 58.8 |
| 21B | 0.012 | >50 | 150 |
| 22A | 0.034 | >50 | 81.1 |
| 22B | 0.02 | >50 | 128 |
| 23A | 0.066 | 32.1 | 113 |
| 23B | 0.24 | 49.3 | >180 |
| 24A | 0.038 | >50 | >180 |
| 24B | 0.02 | >50 | >180 |
| 25A | 0.23 | >50 | >180 |
| 25B | 0.017 | >50 | 89.3 |
| 26A | 0.14 | >50 | 82.9 |
| 26B | 0.020 | >50 | 67.8 |
| 27A | 0.24 | >50 | >180 |
| 27B | 0.018 | >50 | 83.3 |
| 28A | 0.012 | >50 | 50.1 |
| 28B | 0.011 | >50 | 142 |
| 29A | 0.74 | >50 | 102 |
| 29B | 0.81 | >50 | >180 |
| 29C | 0.051 | >50 | 86.1 |
| 29D | 0.059 | >50 | 74.8 |
| 30A | 0.14 | >50 | 29.7 |
| 30B | 0.074 | >50 | 18.9 |
| 30C | 0.31 | >50 | 41.1 |
| 30D | 0.047 | >50 | 15.3 |
| 31A | 0.051 | 31.5 | 114 |
| 31B | 0.019 | 22.7 | 127 |
| 32A | 0.016 | >50 | 101 |
| 32B | 0.088 | >50 | 134 |
| 33A | 0.036 | >50 | >180 |
| 33B | 0.043 | >50 | >180 |
| 34A | 0.63 | >50 | >180 |
| 34B | 0.052 | 34.9 | >180 |
| 35A | 0.20 | >50 | 47 |
| 35B | 0.01 | >50 | >180 |
| 36A | 0.64 | >50 | >180 |
| 36B | 0.029 | >50 | >180 |
| 37A | 0.15 | >50 | >180 |
| 37B | 0.041 | >50 | 53.5 |
| 38A | 0.06 | 47 | 60.8 |
| 38B | 0.012 | 28.4 | 38.5 |
| 39A | 0.13 | >50 | 95.2 |
| 39B | 0.035 | >25 | >180 |
| 40A | 0.054 | 22 | >180 |
| 40B | 0.26 | 22.9 | >180 |
| 41A | 0.076 | >50 | >180 |
| 41B | 0.014 | >50 | 99.1 |
| 42A | 0.12 | >50 | >180 |
| 42B | 0.06 | >50 | >180 |
| 43A | 0.31 | >50 | >180 |
| 43B | 0.065 | >50 | >180 |
| 44A | 0.22 | >50 | >180 |
| 44B | 0.065 | >50 | 133 |
| 45A | 0.57 | >50 | >180 |
| 45B | 0.027 | >50 | >180 |
| 46A | 0.31 | >50 | >180 |
| 46B | 0.031 | >50 | >180 |
| 47A | 0.068 | >50 | 53.3 |
| 47B | 0.016 | >50 | 144 |
| 48A | 0.052 | 22.1 | 133 |
| 48B | 0.028 | 11.5 | 99.5 |
| 49A | 0.041 | >50 | 79.2 |
| 49B | 0.013 | >50 | >180 |
| 50A | 0.066 | 12.5 | >180 |
| 50B | 0.024 | 10.2 | >180 |
| 51A | 0.06 | >50 | >180 |
| 51B | 0.028 | >50 | >180 |
| 52A | 0.12 | >50 | 88.8 |
| 52B | 0.016 | >50 | >180 |
| 53A | 0.24 | >50 | 111 |
| 53B | 0.01 | >50 | 70.9 |
| 54A | 0.16 | >50 | 77.2 |
| 54B | 0.01 | >50 | 117 |
| 55A | 0.051 | >50 | >180 |
| 55B | 0.06 | >50 | >180 |
| 56A | 0.29 | >50 | >180 |
| 56B | 0.084 | >50 | 91.6 |
| 57A | >2.5 | >50 | >180 |
| 57B | 0.65 | >50 | 101 |
| 58A | >2.5 | >50 | >180 |
| 58B | 0.19 | >50 | >180 |
| 59A | 0.51 | >50 | >180 |
| 59B | 0.055 | >50 | >180 |
| 60A | 0.30 | >50 | >180 |
| 60B | 0.052 | >50 | >180 |
| 61A | 2.34 | >50 | >180 |
| 61B | 0.3 | >50 | 135 |
| 62A | 0.61 | >50 | >180 |
| 62B | 0.068 | >50 | 124 |
| 63A | 0.38 | >50 | >180 |
| 63B | 0.052 | >50 | >180 |
| 64A | 0.039 | >50 | 109 |
| 64B | 0.085 | >50 | >180 |
| 65A | 0.034 | >50 | 152 |
| 65B | 0.046 | >50 | 121 |
| 66A | 0.037 | >26.2 | >180 |
| 66B | 0.017 | 19.1 | >180 |
| 67A | N.A. | N.A. | N.A. |
| 67B | N.A. | N.A. | N.A. |
| 68A | N.A. | N.A. | N.A. |
| 68B | N.A. | N.A. | N.A. |
| Ref 1 | 0.095 | >12.5 | 35 |
| Ref 2 | 0.03 | >50 | 37.8 |
| Ref 3 | 0.072 | >50 | 6.6 |
| Ref 4 | 0.076 | >50 | 13.5 |
| Ref 5 | 0.022 | >50 | 7.9 |

N.A. = not available
CC50 values: 3-days incubation unless marked with * (* = 4-days incubation)

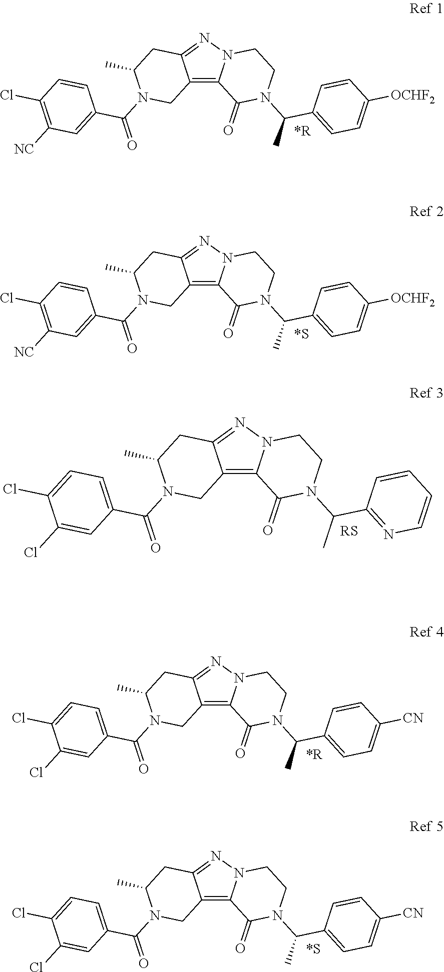 [SMILES: C[C@@H]1Cc2nn3c(c2CN1C(=O)c1ccc(Cl)c(C#N)c1)C(=O)N([C@H](C)c1ccc(OC(F)F)cc1)CC3 ; C[C@@H]1Cc2nn3c(c2CN1C(=O)c1ccc(Cl)c(C#N)c1)C(=O)N([C@@H](C)c1ccc(OC(F)F)cc1)CC3 ; CC(c1ccccn1)N1CCn2nc3c(c2C1=O)CN(C(=O)c1ccc(Cl)c(Cl)c1)[C@H](C)C3 ; C[C@@H]1Cc2nn3c(c2CN1C(=O)c1ccc(Cl)c(Cl)c1)C(=O)N([C@H](C)c1ccc(C#N)cc1)CC3 ; C[C@@H]1Cc2nn3c(c2CN1C(=O)c1ccc(Cl)c(Cl)c1)C(=O)N([C@@H](C)c1ccc(C#N)cc1)CC3]

Ref 1, Ref 2, Ref 3, Ref 4 and Ref 5 can be prepared according to the procedures of WO2020/243135.

According to the experimental results, it can be seen that the present compounds showed improved human liver microsome stability as well as reasonable anti-HBV activity. As compared to comparative compounds (e.g., 1A and 1B and reference compounds 1 and 2, as well as 3-5), the half-life (t_{1/2}) of the present compounds are significantly increased, showing great improvement in human metabolic stability.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A compound of Formula (I), 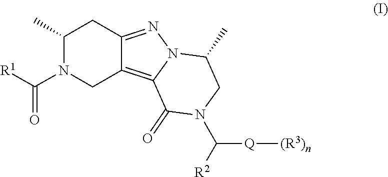
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl, C₃₋₆cycloalkyl and CON(R_{S})₂;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, oxo, and N(R_{S})₂, each of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂ is optionally substituted with 1, 2, 3, 4 or 5 substituents, each of said substituents independently selected from the group consisting of halo, hydroxyl, C₁₋₆alkyl and oxo;
R_{S} is each independently selected from the group consisting of H, C₁₋₆alkyl, CN, SOC₁₋₆alkyl, SO₂C₁₋₆alkyl, SO₂OH, C₃₋₆cycloalkyl;
or a pharmaceutically acceptable salt or a solvate thereof.

2. The compound of claim 1, wherein R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₃₋₆cycloalkyl, CN, CF₃, CHF₂, OCHF₂ and OCF₃.

3. The compound of claim 2, wherein R¹ is phenyl or pyridyl, which is substituted with 1, 2 or 3 substituents, each of said substituents independently selected from the group consisting of halo, CN and CF₃.

4. The compound of claim 1, wherein the structural unit 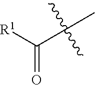
in Formula (I) satisfies Formula (Ia) 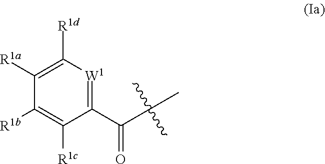
wherein R^{1a}, R^{1b}, R^{1c} and R^{1d} are each independently selected from the group consisting of hydrogen, halo, C₁₋₆alkyl, C₃₋₆cycloalkyl, CN, CF₃, CHF₂, OCHF₂ and OCF₃;
W¹ is N or CH;
with at least one of R^{1a}, R^{1b}, R^{1c} and R^{1d} not being hydrogen.

5. The compound of claim 4, wherein
R^{1a} is halo; R^{1b} and R^{1d} are each independently selected from the group consisting of hydrogen, halo, cyano, CF₃, OCHF₂ and OCF₃; and R^{1c} is hydrogen or halo.

6. The compound of claim 1, wherein
R² is selected from the group consisting of CHF₂, CF₃, C₁₋₆alkyl, C₁₋₆alkylOC₁₋₆alkyl, C₃₋₆cycloalkyl and CON(R_{S})₂; and
the structure of Formula (I) has Formula (I-1) or Formula (I-2)

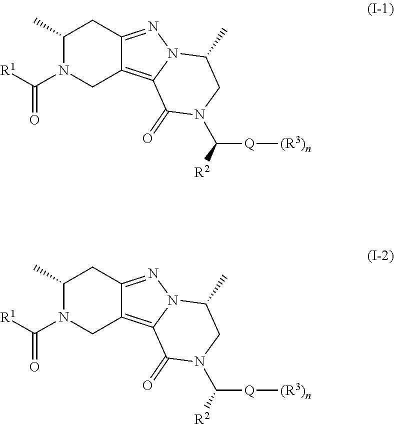

7. The compound of claim 1, wherein R² is methyl or CONHCH₃.

8. The compound of claim 1, wherein Q is a ring selected from the group consisting of phenyl, pyridyl, pyrimidinyl, pyridazinyl, pyrazinyl, pyrazolyl, and oxadiazolyl.

9. The compound of claim 1, wherein the structural unit 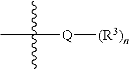
in Formula (I) satisfies Formula (Ib) 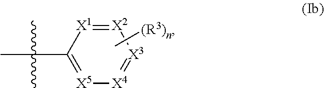
wherein
all of X¹, X², X³, X⁴ and X⁵ are CH; or
one or two of X¹, X², X³, X⁴ and X⁵ are N, and rest of them are CH.

10. The compound of claim 9, wherein the structural unit 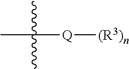
of Formula (I) satisfies Formula (Ic) 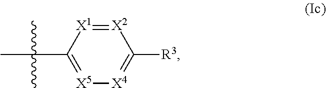
wherein
all of X¹, X², X⁴ and X⁵ are CH;
X² is N, and X¹, X⁴ and X⁵ are CH; or
X¹ is N, and X², X⁴ and X⁵ are CH.

11. The compound of claim 9, wherein
both of X¹ and X² are N, and X⁴ and X⁵ are CH;
both of X² and X⁴ are N, and X¹ and X⁵ are CH;
both of X¹ and X⁴ are N, and X² and X⁵ are CH; or
both of X¹ and X⁵ are N, and X² and X⁴ are CH.

12. The compound of claim 1, wherein the structural unit 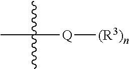
in Formula (I) satisfies Formula (Ib′) 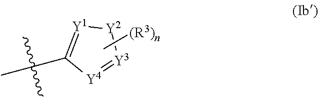
wherein one, two or three of Y¹, Y², Y³ and Y⁴ are N or NH or O, and rest of them are CH.

13. The compound of claim 1, wherein
Y¹ and Y² are N or NH, and Y³ and Y⁴ are CH; or
Y¹ and Y² are N or NH, Y⁴ is O, and Y³ is CH.

14. The compound of claim 1, wherein halo is F, Cl or Br.

15. The compound of claim 1, wherein n is 1, 2 or 3.

16. The compound of claim 1, wherein R³ is independently selected from the group consisting of OCHF₂, CHF₂, CH₂CF₃, C(CH₃)₂OH, CH₂C(CH₃)₂OH, cyclopropyl, CH₃, CF₃, 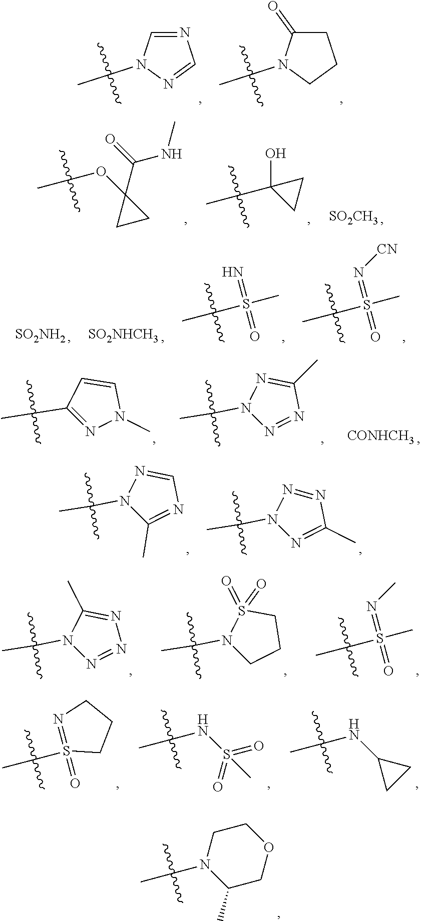
halo, oxo, isopropyl, O-isopropyl, and CN.

17. A compound of claim 1, selected from the group consisting of the following compounds,
or a stereoisomeric or a tautomeric form thereof: 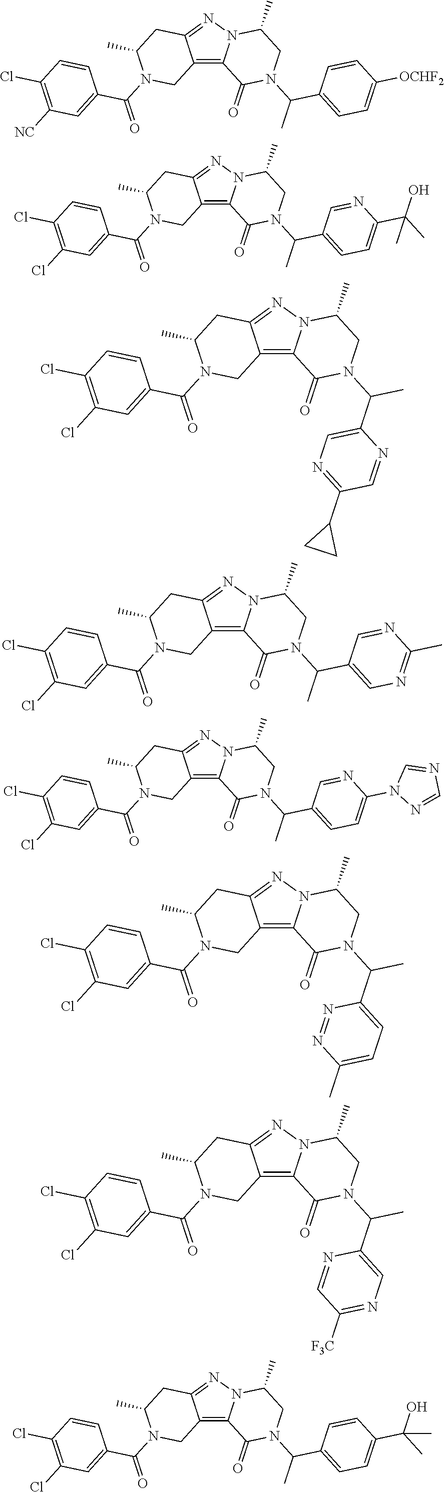 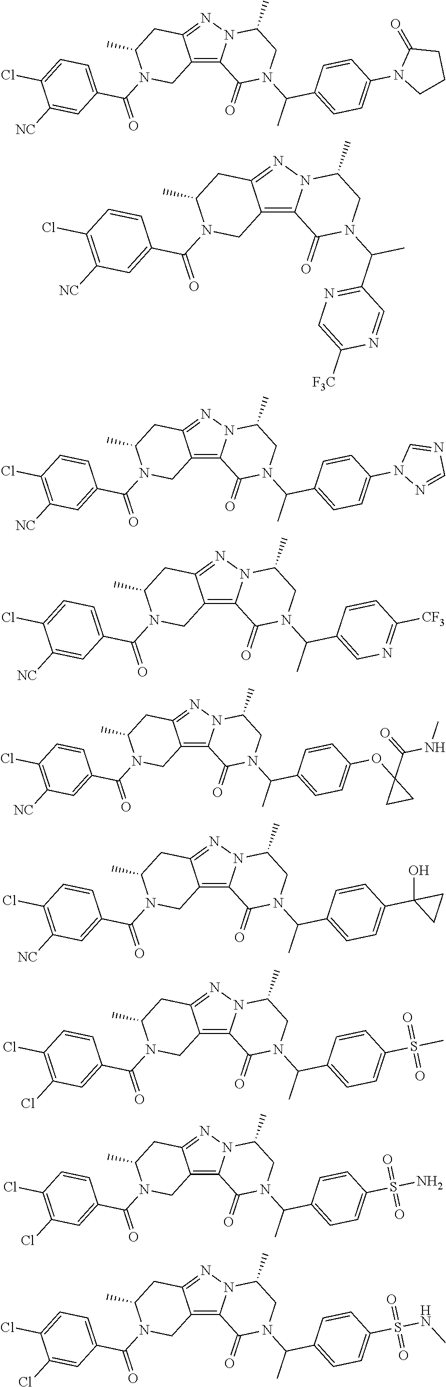 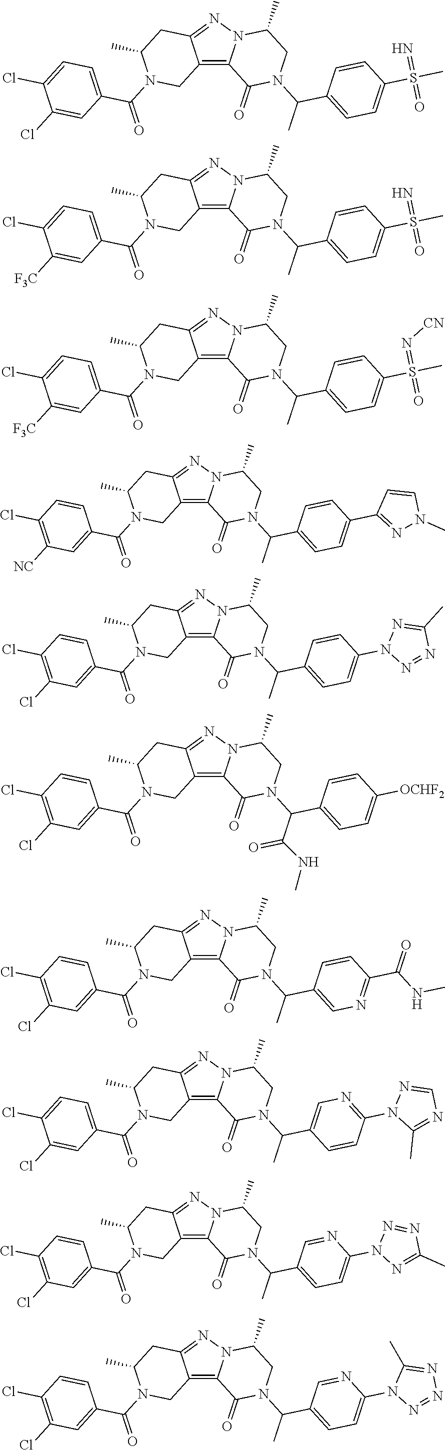 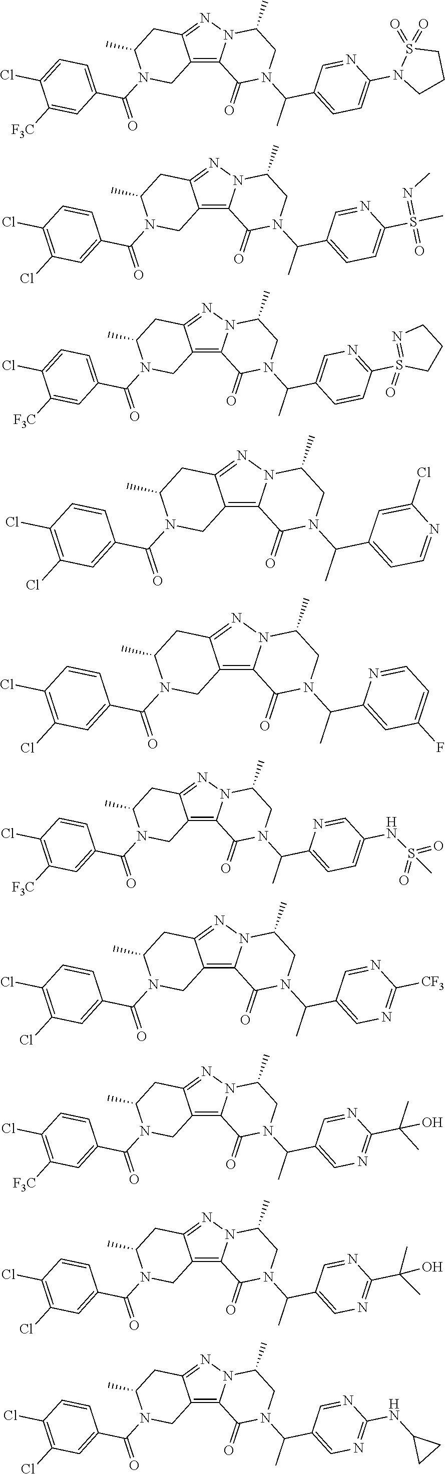 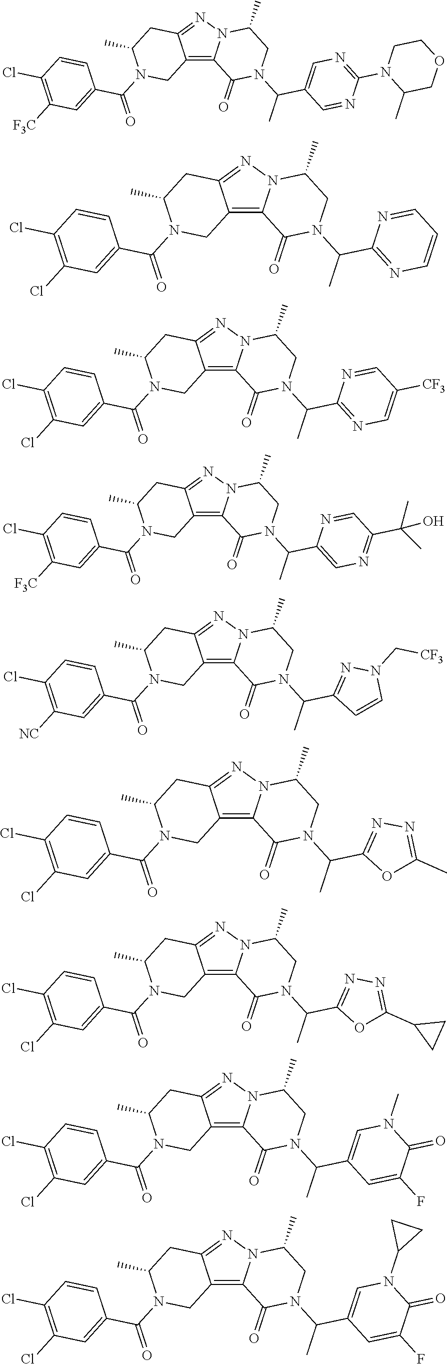 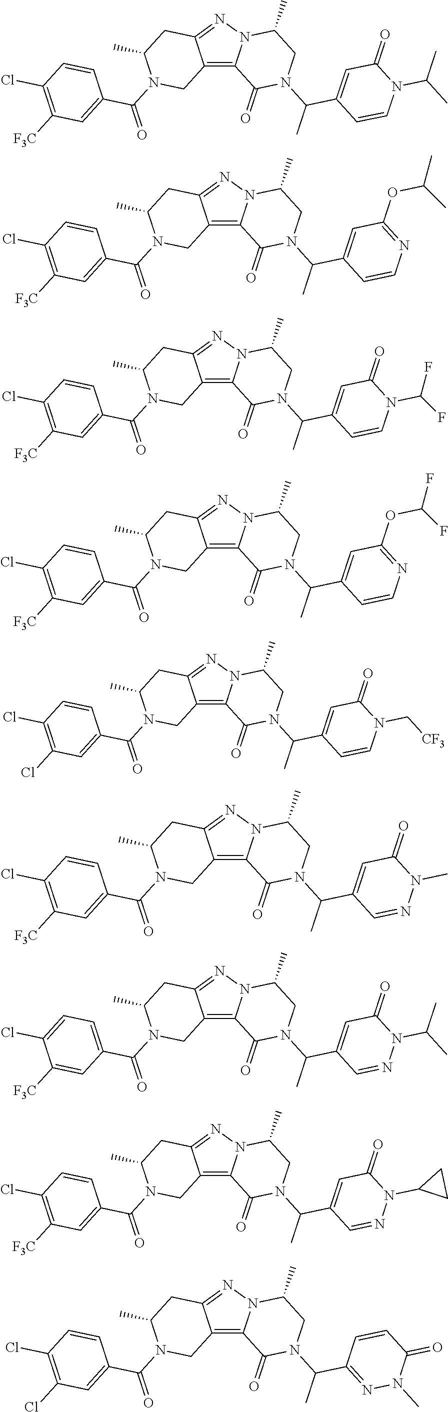 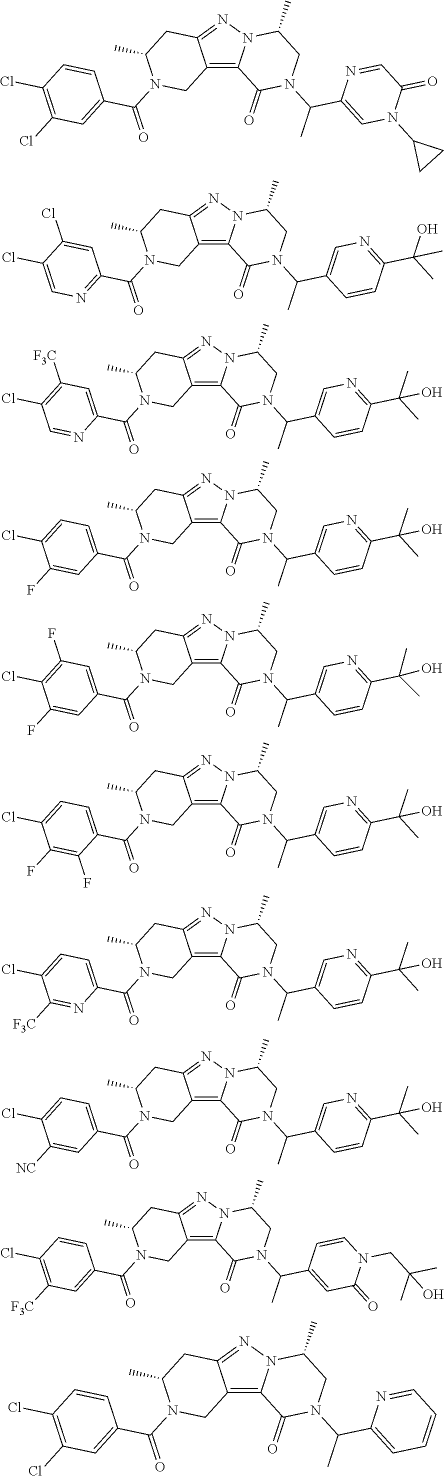 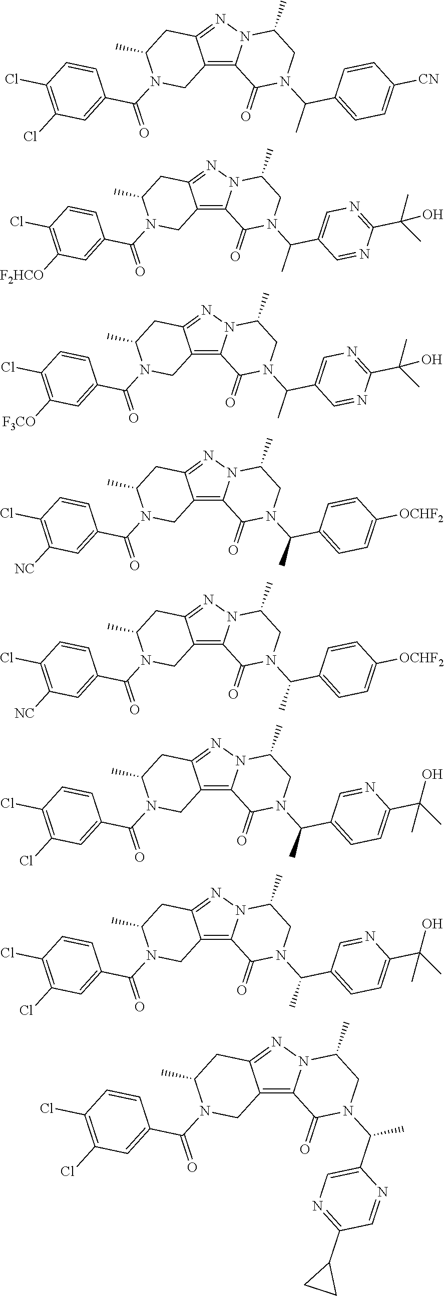 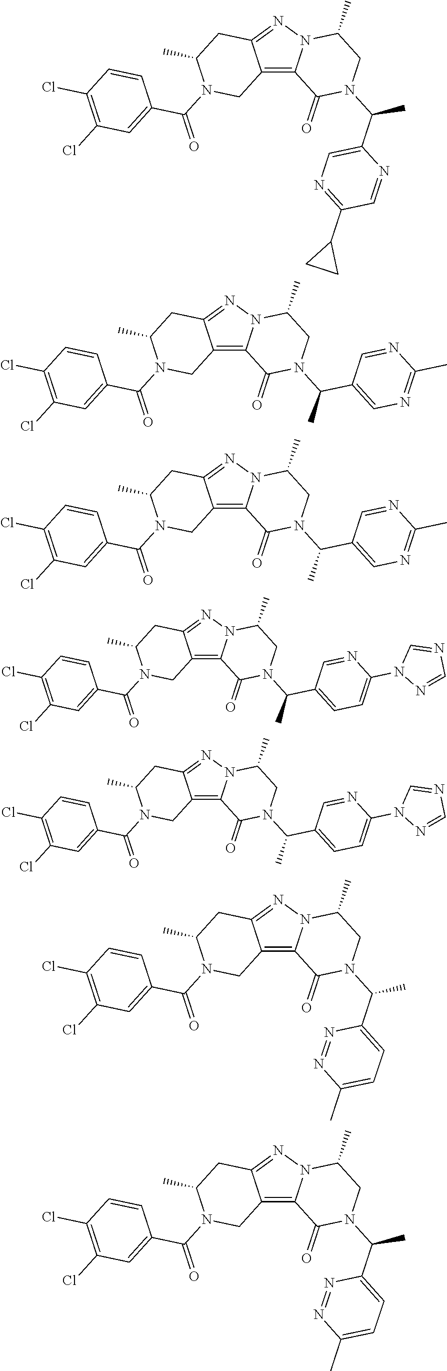 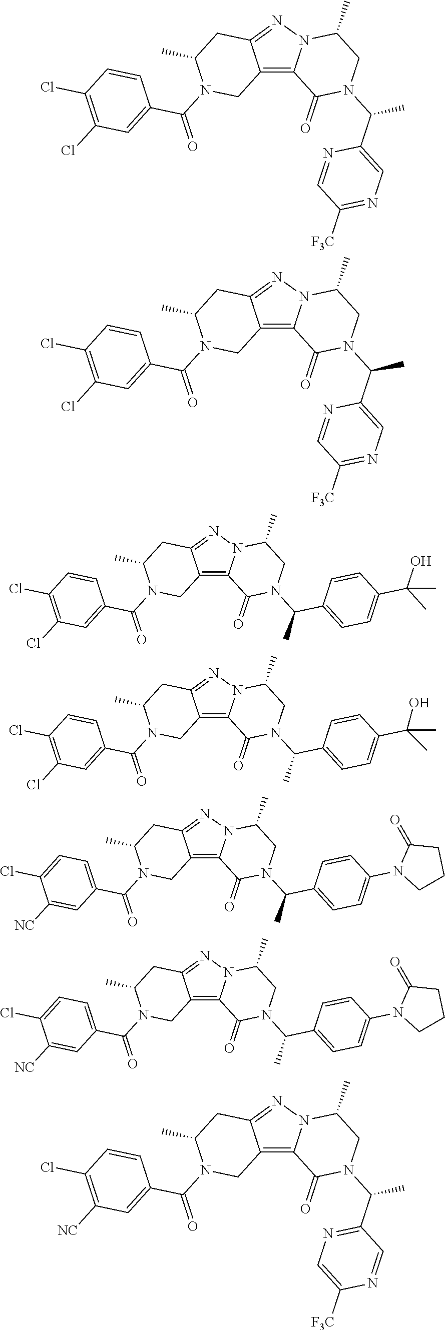 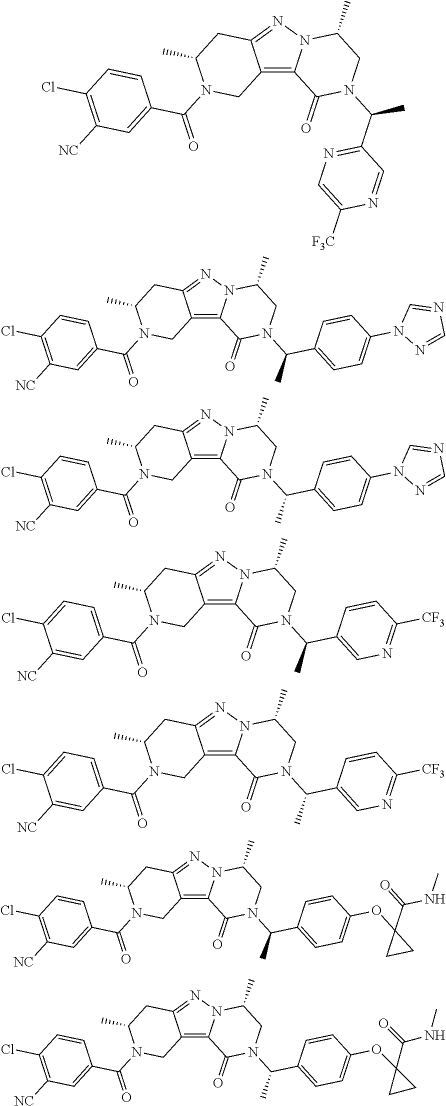 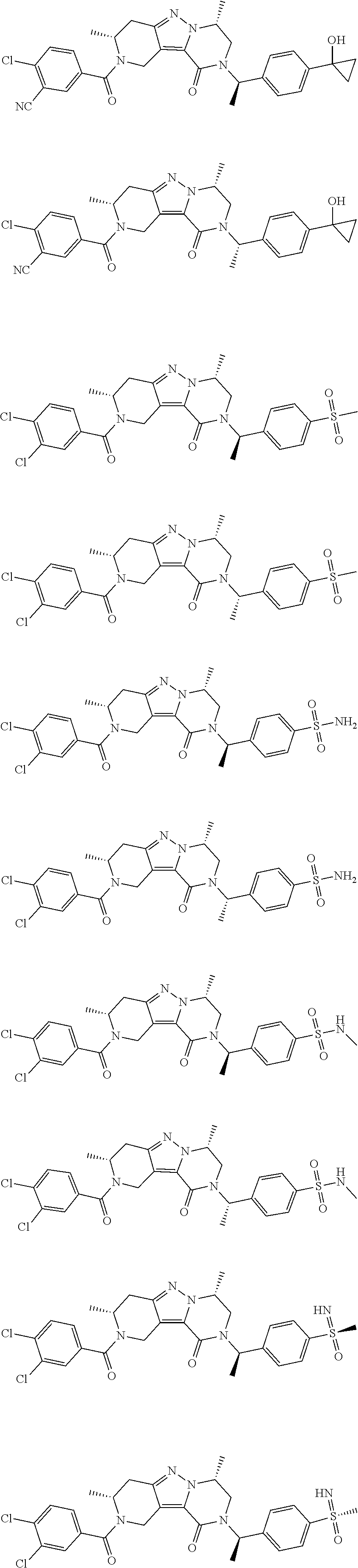 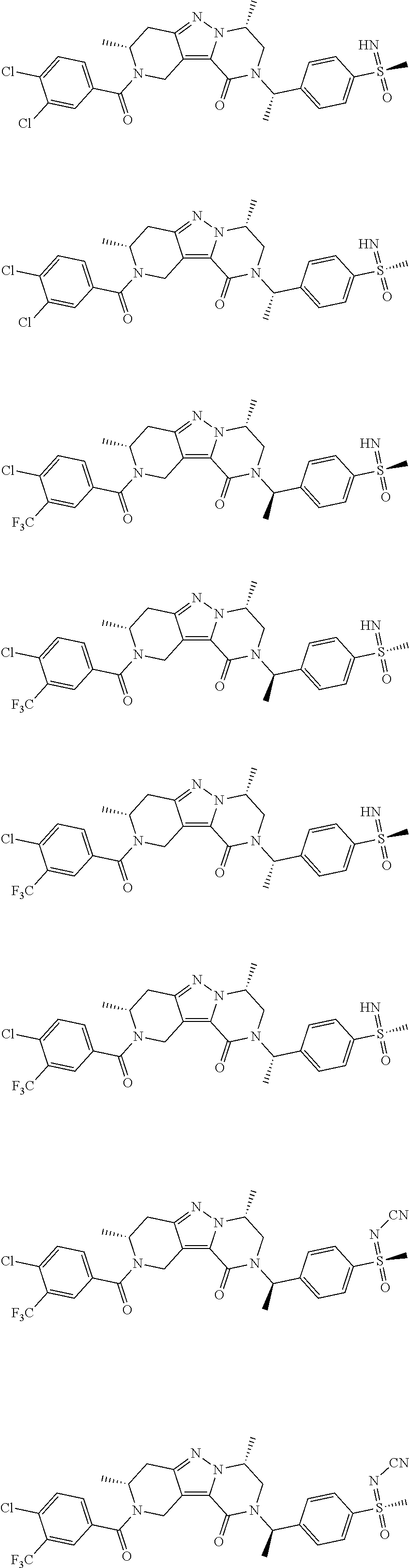 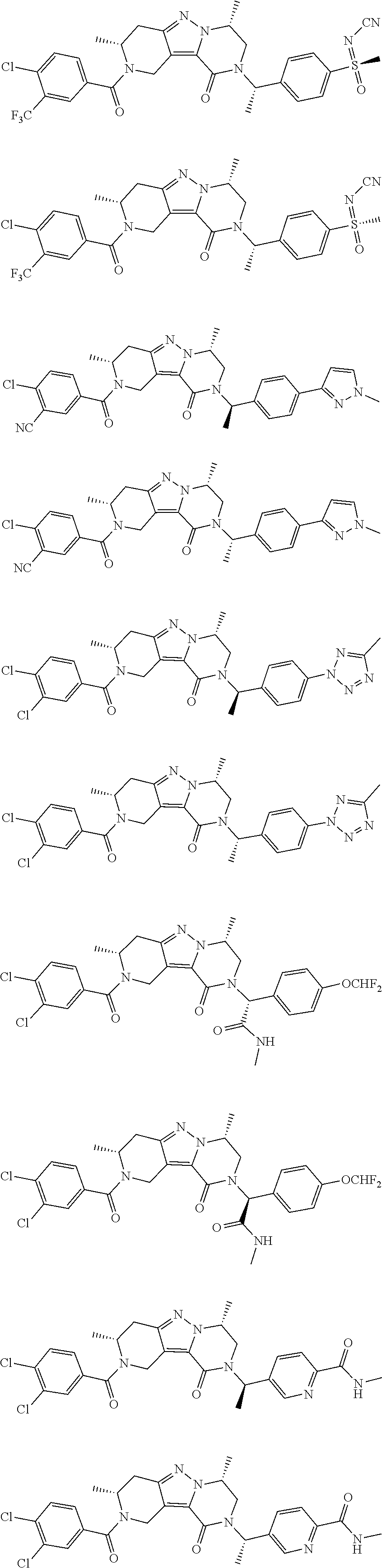 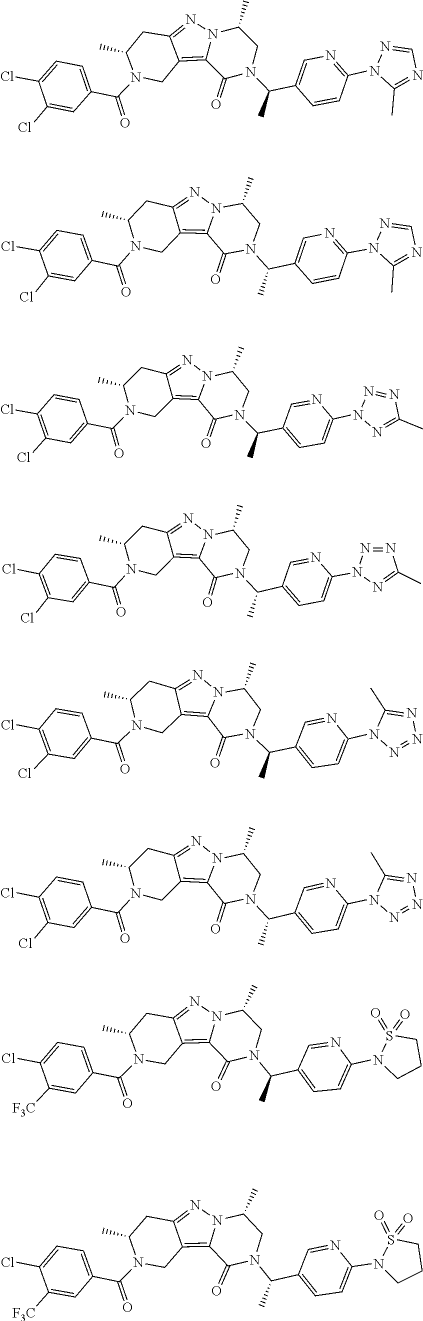 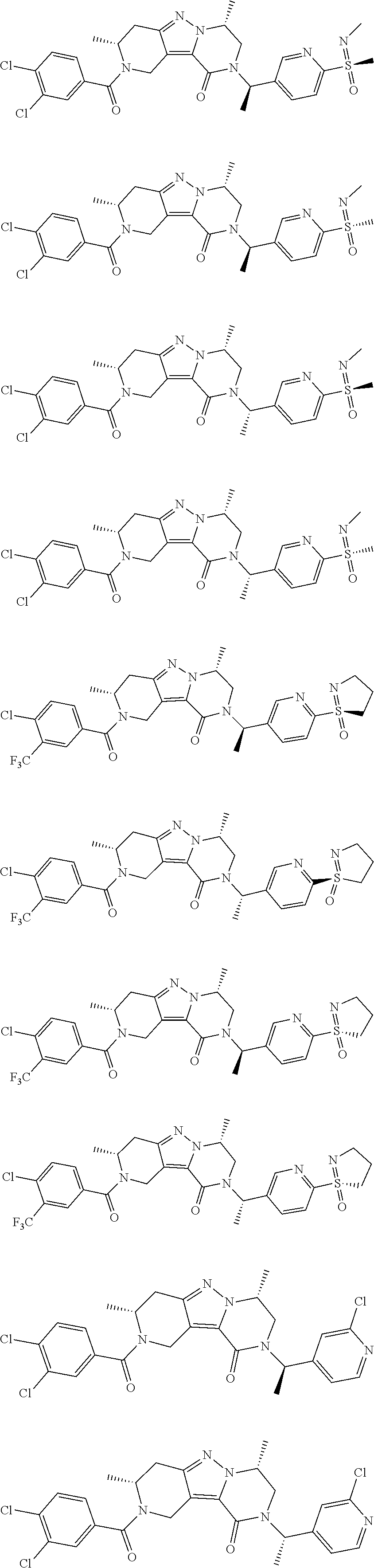 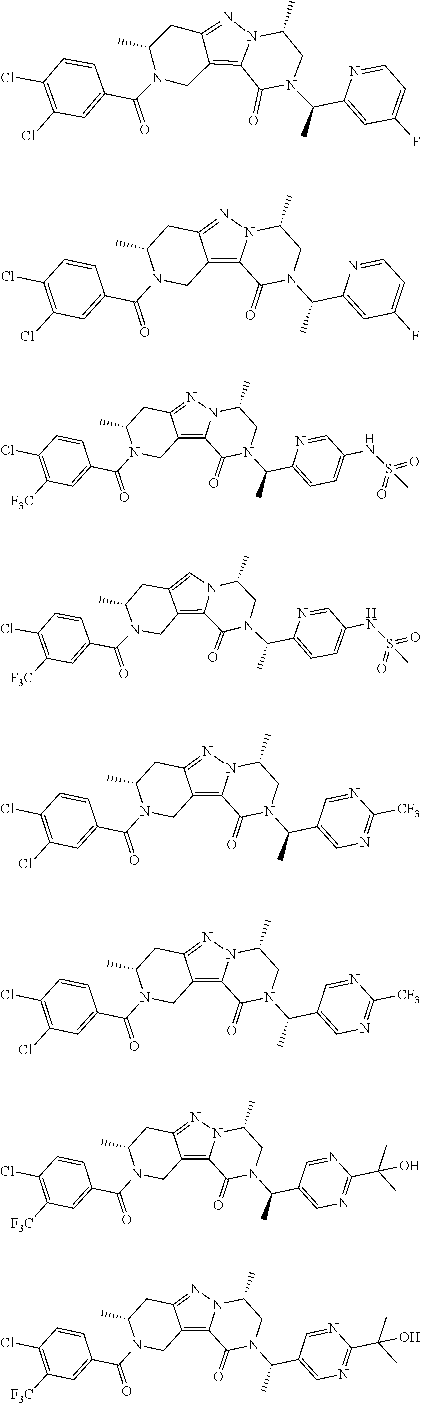 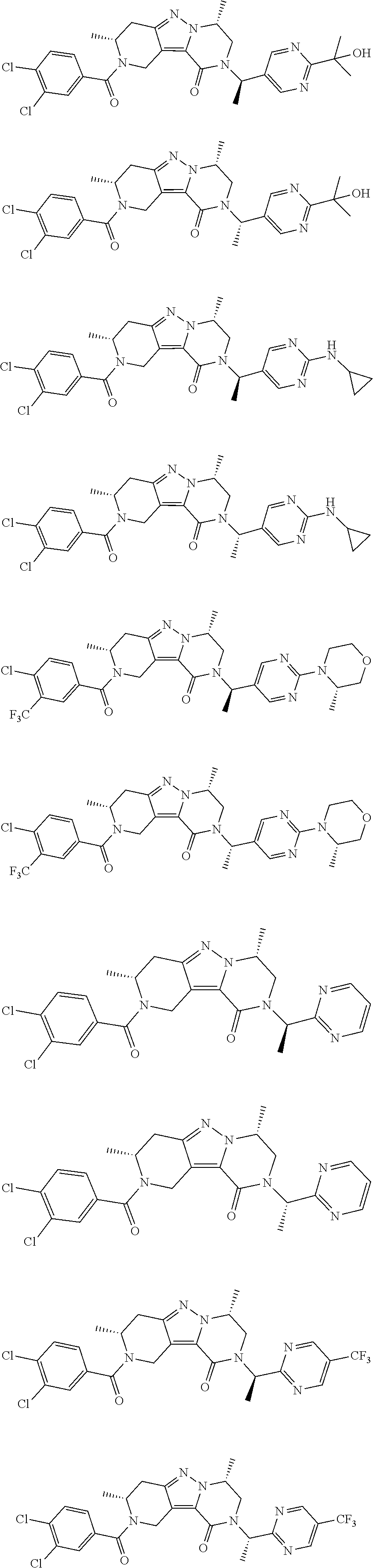 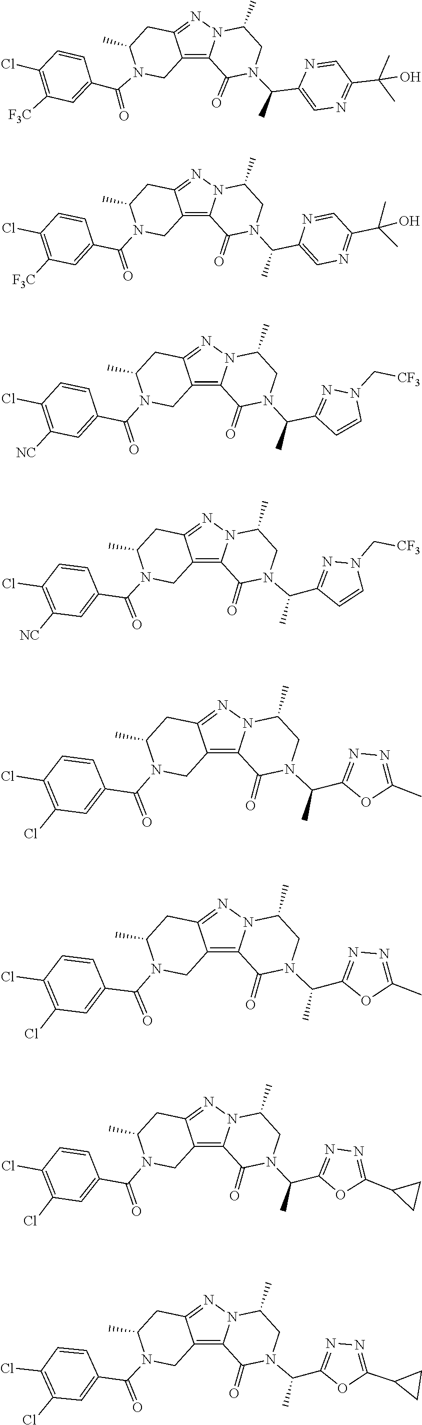 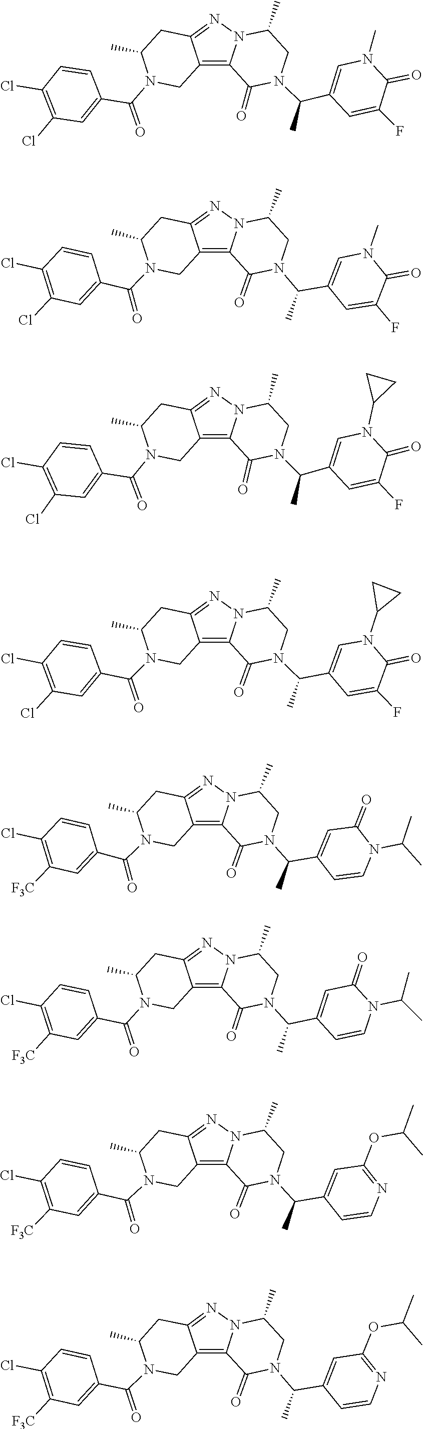 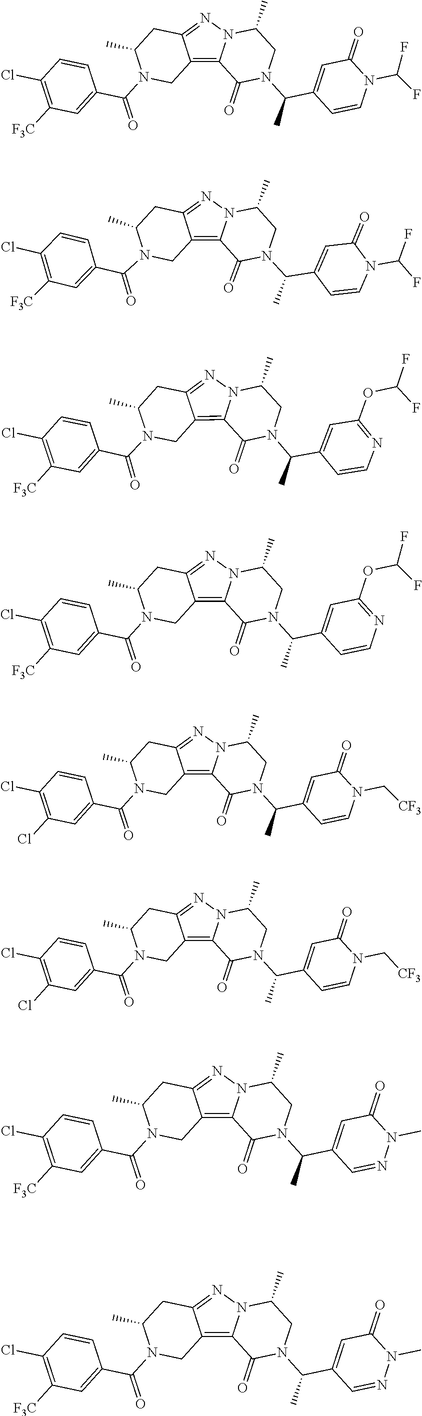 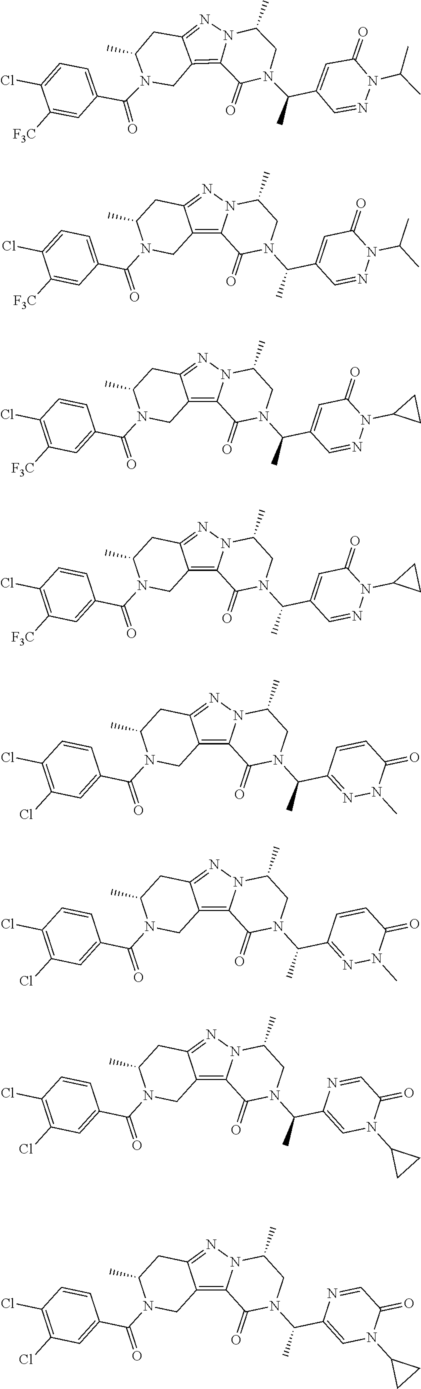 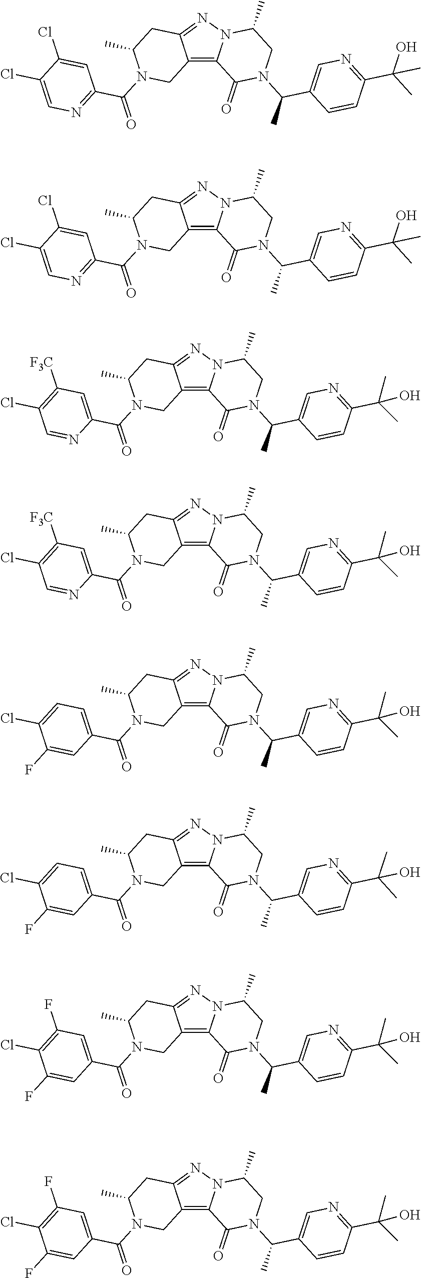 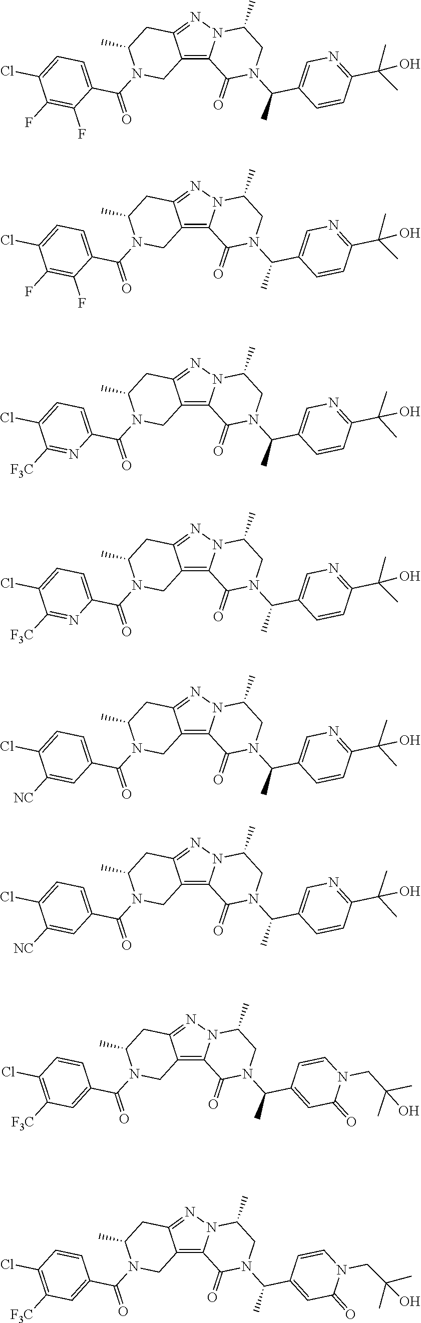 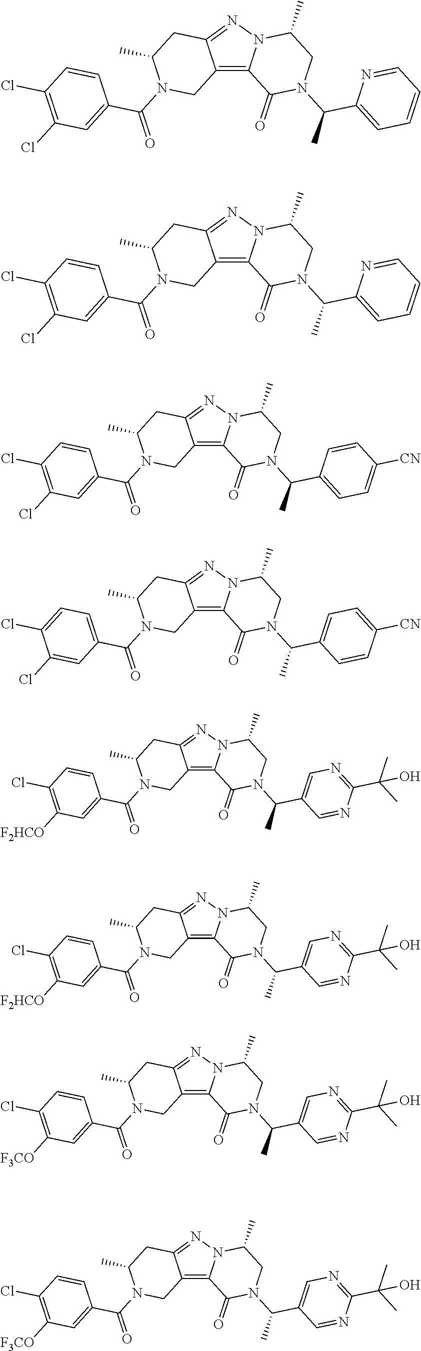
or a pharmaceutically acceptable salt, N-oxide, or a solvate thereof.

18. (canceled)

19. A pharmaceutical composition, which comprises the compound of claim 1, and which further comprises at least one pharmaceutically acceptable excipient.

20-22. (canceled)

23. A method of treating an HBV infection or an HBV-induced disease in an individual in need thereof, comprising administering to the individual a therapeutically effective amount of a compound of Formula (I), 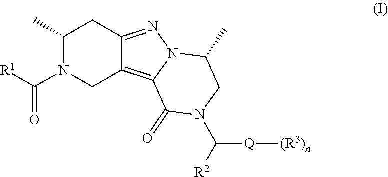
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl, C₃₋₆cycloalkyl and CON(R_{S})₂;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, oxo, and N(R_{S})₂, each of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂ is optionally substituted with 1, 2, 3, 4 or 5 substituents, each of said substituents independently selected from the group consisting of halo, hydroxyl, C₁₋₆alkyl and oxo;
R_{S} is each independently selected from the group consisting of H, C₁₋₆alkyl, CN, SOC₁₋₆alkyl, SO₂C₁₋₆alkyl, SO₂OH, C₃₋₆cycloalkyl;
or a pharmaceutically acceptable salt or a solvate thereof.

24. A product comprising a first compound and a second compound as a combined preparation for simultaneous, separate or sequential use in the prevention or treatment of an HBV infection or of an HBV-induced disease in a subject in need thereof, wherein said first compound is different from said second compound, wherein said first compound is a compound of Formula (I), 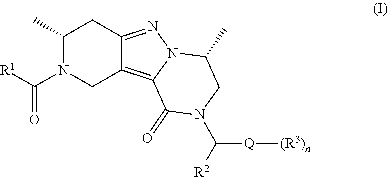
or a stereoisomeric or a tautomeric form thereof, wherein
R¹ is selected from the group consisting of phenyl, 5-membered heteroaryl and 6-membered heteroaryl, each of which is substituted with 1, 2 or 3 substituents, each of said substituents independently selected from the group consisting of halo, C₁₋₆alkyl, C₁₋₆alkoxyl, C₃₋₆cycloalkyl and CN, each of C₁₋₆alkyl, C₁₋₆alkoxyl and C₃₋₆cycloalkyl is optionally substituted with 1, 2, 3, 4 or 5 substituents independently selected from the group consisting of halo, hydroxyl and CN;
R² is selected from the group consisting of H, CHF₂, CF₃, C₁₋₆alkyl, C₁₋₆alkylOC₁₋₆alkyl, C₃₋₆cycloalkyl and CON(R_{S})₂;
Q represents a ring selected from the group consisting of phenyl, 5-membered heteroaryl and 6-membered heteroaryl;
n represents 0, 1, 2 or 3;
each R³ independently represents a substituent selected from the group consisting of CN, C₁₋₆alkyl, C₁₋₆alkoxyl, C₃₋₆cycloalkyl, 5-membered heteroaryl, 6-membered heteroaryl, 4-8 membered heterocyclyl, halo, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, oxo, and N(R_{S})₂, each of C₁₋₆alkyl, C₁₋₆alkoxyl, C₃₋₆cycloalkyl, 5-membered heteroaryl, 6-membered heteroaryl, 4-8 membered heterocyclyl, O—C₃₋₆cycloalkyl-CON(R_{S})₂, SOC₁₋₆alkyl, SO₂C₁₋₆alkyl, SON(R_{S})₂, SO₂N(R_{S})₂, SO(C₁₋₆alkyl)NRS, CON(R_{S})₂, and N(R_{S})₂ is optionally substituted with 1, 2, 3, 4 or 5 substituents, each of said substituents independently selected from the group consisting of halo, hydroxyl, C₁₋₆alkyl and oxo;
R_{S} is each independently selected from the group consisting of H, C₁₋₆alkyl, CN, SOC₁₋₆alkyl, SO₂C₁₋₆alkyl, SO₂OH, C₃₋₆cycloalkyl;
or a pharmaceutically acceptable salt or a solvate thereof, and wherein said second compound is another HBV inhibitor.

25-26. (canceled)


SEQUENCE LISTING (3 sequences)

SEQ ID NO: 1 - DNA, 20 bp, Artificial Sequence
  Forward Primer
gtgtctgcggcgttttatca

SEQ ID NO: 2 - DNA, 21 bp, Artificial Sequence
  Reverse Primer
gacaaacgggcaacatacctt

SEQ ID NO: 3 - DNA, 28 bp, Artificial Sequence
  Taqman Probe
cctctkcatcctgctgctatgcctcatc